(19) United States
(12) Patent Application Publication
(10) Pub. No.: US 2024/0317815 A1
(43) Pub. Date: Sep. 26, 2024

(54) T-CELL MODULATORY MULTIMERIC POLYPEPTIDES AND METHODS OF USE THEREOF

(71) Applicant: Cue Biopharma, Inc., Boston, MA (US)

(72) Inventors: Anish Suri, Boston, MA (US); Raymond J. Moniz, Boston, MA (US)

(21) Appl. No.: 18/736,261

(22) Filed: Jun. 6, 2024

Related U.S. Application Data
(63) Continuation of application No. PCT/US22/80263, filed on Nov. 21, 2022.
(60) Provisional application No. 63/283,028, filed on Nov. 24, 2021.
(60) Provisional application No. 63/309,288, filed on Feb. 11, 2022.
(60) Provisional application No. 63/338,131, filed on May 4, 2022.

(51) Int. Cl. C07K 14/005; A61K 38/00; C07K 14/55; C07K 14/74; C07K 16/28

(52) U.S. Cl. CPC C07K 14/005; C07K 14/55; C07K 14/70539; C07K 16/2803; A61K 38/00; C07K 2317/52; C07K 2317/622; C12N 2770/20022

(57) ABSTRACT

The present disclosure provides T-cell modulatory multimeric polypeptides (TMMPs) that comprise (i) an optional immunomodulatory polypeptide such as a variant IL-2 polypeptide, (ii) class I HLA major histocompatibility complex (MHC) polypeptides (a class I HLA heavy chain polypeptide and a β2 microglobulin polypeptide), (iii) a peptide that presents an epitope to a T-cell receptor, which together with the class I MHC polypeptides forms a peptide-MHC complex (pMHC), (iv) a tumor-targeting polypeptide, and (v) an optional Ig Fc polypeptide or other scaffold. Such TMMP is useful for modulating the activity of a T cell, and for modulating an immune response in an individual, and for “redirecting” a patient's repertoire of antiviral T cells to attack and kill cancer cells.

### CROSS-REFERENCE

This application claims the benefit of U.S. Provisional Patent Application No. 63/283,028, filed Nov. 24, 2021, U.S. Provisional Patent Application No. 63/309,288, filed Feb. 11, 2022, and U.S. Provisional Patent Application No. 63/338,131, filed May 4, 2022, which applications are incorporated herein by reference in their entirety.

### INCORPORATION-BY-REFERENCE OF MATERIAL ELECTRONICALLY SUBMITTED

A Sequence Listing is provided herewith as a Sequence Listing XML, “CUEB-145WO_SEQUENCE_LIST” created on Nov. 17, 2022 and having a size of 1,190,000 bytes. The contents of the Sequence Listing XML are incorporated by reference herein in their entirety.

### INTRODUCTION

An adaptive immune response involves the engagement of the T cell receptor (TCR), present on the surface of a T cell, with a small peptide antigen non-covalently presented on the surface of an antigen presenting cell (APC) by a major histocompatibility complex (MHC; also referred to in humans as a human leukocyte antigen (HLA) complex). This engagement represents the immune system's targeting mechanism and is a requisite molecular interaction for T cell modulation (activation or inhibition) and effector function. Following epitope-specific cell targeting, the targeted T cells are activated through engagement of costimulatory proteins found on the APC with counterpart costimulatory proteins the T cells. Both signals—epitope/TCR binding and engagement of APC costimulatory proteins with T cell costimulatory proteins—are required to drive T cell specificity and activation or inhibition. The TCR is specific for a given epitope; however, the costimulatory protein not epitope specific and instead is generally expressed on all T cells or on large T cell subsets.

### SUMMARY

The present disclosure provides T-cell modulatory multimeric polypeptides (TMMPs) that comprise an immunomodulatory polypeptide, class I HLA polypeptides (a class I HLA heavy chain polypeptide and a β2 microglobulin polypeptide), a peptide that presents an epitope to a T-cell receptor, and a tumor-targeting polypeptide. A TMMP of the present disclosure is useful for modulating the activity of a T cell, and for modulating an immune response in an individual and for “redirecting” a patient's repertoire of antiviral T cells to attack and kill cancer cells.

### BRIEF DESCRIPTION OF THE DRAWINGS

FIG. 1A-1B provide an amino acid sequence of a wild-type human β2M polypeptide (SEQ ID NO:1) (FIG. 1A) and an amino acid sequence of a β2M polypeptide with an R12C substitution (SEQ ID NO:2) (FIG. 1B).

FIGS. 2A-2M provide amino acid sequences of immunoglobulin Fc polypeptides (SEQ ID Nos: 3-15, respectively).

FIG. 3A-3E provide amino acid sequences of wild-type HLA-A*0201 (SEQ ID NO:16) (FIG. 3A) and variants (SEQ ID Nos: 17-20, respectively) (FIG. 3B-3E).

FIG. 4A-4E provide amino acid sequences of wild-type HLA-A*1101 (SEQ ID NO:21) (FIG. 4A) and variants (SEQ ID Nos: 22-25, respectively) (FIG. 4B-4E).

FIG. 5A-5E provide amino acid sequences of wild-type HLA-A*2402 (SEQ ID NO:26) (FIG. 5A) and variants (SEQ ID Nos: 27-30, respectively) (FIG. 5B-5E).

FIG. 6A-6E provide amino acid sequences of wild-type HLA-A*3303 (SEQ ID NO:31) (FIG. 6A) and variants (SEQ ID Nos: 32-35, respectively) (FIG. 6B-6E).

FIGS. 7A-7B provide an alignment of HLA-A heavy chain amino acid sequences (FIG. 7A; SEQ ID NOs:36-44, respectively) and a consensus sequence (FIG. 7B; SEQ ID NO: 45).

FIGS. 8A-8B provide an alignment of HLA-B heavy chain amino acid sequences (FIG. 8A; SEQ ID NOs:46-52, respectively) and a consensus sequence (FIG. 8B; SEQ ID NO: 53).

FIGS. 9A-9B provide an alignment of HLA-C heavy chain amino acid sequences (FIG. 9A; SEQ ID NOs:54-61, respectively) and a consensus sequence (FIG. 9B; SEQ ID NO: 62).

FIG. 10 provides a consensus amino acid sequence for each of HLA-E, -F, and -G heavy chains (SEQ ID NOs: 63-65, respectively). Variable amino acid (aa) positions are indicated as “X” residues sequentially numbered; the locations of amino acids 84, 139, and 236 are double underlined.

FIG. 11 provides an alignment of consensus amino acid sequences for HLA-A (SEQ ID No:45), -B (SEQ ID NO:53), -C(SEQ ID NO:62), -E (SEQ ID NO:63), -F (SEQ ID NO:64), and -G (SEQ ID NO:65).

FIGS. 12A-12D provide amino acid sequences of a wild-type IL-2 polypeptide (SEQ ID NO:66) (FIG. 12A), IL-2Ra (SEQ ID NO:67) (FIG. 12B), IL-2Rβ (SEQ ID NO:68) (FIG. 12C), and IL-2Ry (SEQ ID NO:69) (FIG. 12D).

FIG. 13A-13K provide amino acid sequences of SARS-CoV-2-encoded polypeptides (SEQ ID Nos:70-94).

FIG. 14 provides CD8 T cell epitopes of SARS-CoV-2-encoded polypeptides and primary HLA class I heavy chain allele restriction.

FIG. 15 provides CD8 T cell epitopes of SARS-CoV-2-encoded polypeptides and HLA class I heavy chain allele restriction.

FIG. 16A-16D provide amino acid sequences of HLA-E heavy chains (SEQ ID Nos:837-840, respectively).

FIG. 17A-17D provide amino acid sequences of HLA-G heavy chains (SEQ ID Nos:841-844, respectively).

FIG. 18A-18D provide schematic depictions of TMMPs.

FIG. 19 provides amino acid sequences of SARS-CoV-2 peptides and HLA class I heavy chain allele restriction.

FIG. 20A-20J provide amino acid sequences (FIG. 20A (SEQ ID NO:867), 20C (SEQ ID NO:868), 20E (SEQ ID NO:869), 20G (SEQ ID NO:870), and 20I (SEQ ID NO:871)) and descriptions (FIGS. 20B, 20D, 20F, 20H, and 20J) of exemplary TMMP “heavy chain” constructs.

FIG. 21A-21E provide amino acid sequences and descriptions of exemplary TMMP “light chain” constructs.

FIG. 22A-22H provide amino acid sequences of exemplary anti-mesothelin scFv.

FIG. 23A-23D provide amino acid sequences of exemplary anti-TROP-2 scFv.

FIG. 24A-24B provide amino acid sequences of exemplary “mAb fusion” constructs.

FIG. 25A-25B provide amino acid sequences of exemplary “mAb fusion” constructs.

FIG. 26A-26B provide amino acid sequences of exemplary “mAb fusion” constructs.

FIG. 27A-27B provide amino acid sequences of exemplary anti-CD28 scFv.

FIG. 28A-28C provide schematic depictions of TMMPs, showing various possible MOD positions and various possible TTP positions.

FIG. 29 depicts PBMC reactivity to the SARS-CoV-2 HLA-A*0201 peptide YLQPRTFLL (SEQ ID NO:218).

FIG. 30 depicts expansion of SARS-CoV-2 (SCV2)-reactive CD8⁺ T cells.

FIG. 31A-31D depict the effect of various constructs on activation of SARS-CoV2-specific T cells present in a population of PBMCs (FIG. 31A and FIG. 31B) and PBMC-mediated killing of target B cells (FIG. 31C and FIG. 31D).

FIG. 32A-32G provide amino acid sequences of polypeptide chains of an exemplary TMMP, where FIG. 32A provides the amino acid sequence of the polypeptide designated “4518” (SEQ ID NO:867), FIG. 32B provides the amino acid sequence of the polypeptide designated “4700” (SEQ ID NO:883), and FIG. 32C-32G provide amino acid sequences of components of the 4518 and 4700 polypeptides.

FIG. 33 provides the amino acid sequence of the polypeptide designated “4656” (SEQ ID NO:910)

FIG. 34 provides the amino acid sequence of the polypeptide designated “3534” (SEQ ID NO:911).

FIG. 35 provides the amino acid sequence of the polypeptide designated “1717” (SEQ ID NO:912).

FIG. 36A-36B depict the effect of various constructs on expansion of SARS-CoV2-specific CD8⁺ T cells (FIG. 36A) or CMV-specific CD8⁺ T cells present in a population of PBMCs.

FIG. 37A-37B depict the effect of various constructs on killing of CD19⁺ tumor cells.

FIG. 38A-38L provide amino acid sequences of various polypeptides suitable for including in a TMMP (FIG. 38A-38H) (SEQ ID Nos: 1209-1216, respectively) and certain components of the polypeptides (FIG. 38I-38L) (SEQ ID NOs: 872, 18, 877, and 878 respectively).

FIG. 39A-39B provide examples of amino acid sequences of polypeptides suitable for including in a TMMP (SEQ ID Nos: 1217 and 1218, respectively).

FIG. 40A-40B provide examples of amino acid sequences of polypeptides suitable for including in a TMMP (SEQ ID Nos: 1219 and 1218, respectively).

FIG. 41A-41C provide examples of amino acid sequences of polypeptides suitable for including in a TMMP (SEQ ID Nos: 870, 1220 and 883, respectively).

FIG. 42A-42C provide examples of amino acid sequences of polypeptides suitable for including in a TMMP (SEQ ID Nos: 1221-1223, respectively).

FIGS. 43A and 43B provide amino acid sequences of constructs 1715 (FIG. 43A) (SEQ ID NO:1224) and 1717 (FIG. 43B) (SEQ ID NO:912).

FIG. 44 depicts the effect of linker length on TMMP-mediated target tumor cell killing.

FIG. 45 depicts the effect of copy number of variant IL-2 polypeptide on TMMP-mediated target tumor cell killing.

FIG. 46 depicts TMMP-mediated anti-viral T cell expansion.

### DEFINITIONS

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones. Furthermore, as used herein, a “polypeptide” refers to a protein that includes modifications, such as deletions, additions, and substitutions (generally conservative in nature as would be known to a person in the art) to the native sequence, as long as the protein maintains the desired activity. These modifications can be deliberate, as through site-directed mutagenesis, or can be accidental, such as through mutations of hosts that produce the proteins, or errors due to polymerase chain reaction (PCR) amplification or other recombinant DNA methods. References herein to a specific residue or residue number in a known polypeptide are understood to refer to the amino acid at that position in the wild-type polypeptide. To the extent that the sequence of the wild-type polypeptide is altered, either by addition or deletion of one or more amino acids, one of ordinary skill will understand that a reference to the specific residue or residue number will be correspondingly altered so as to refer to the same specific amino acid in the altered polypeptide, which would be understood to reside at an altered position number. For example, if an MHC class I polypeptide is altered by the addition of one amino acid at the N-terminus, then a reference to position 84 or a specific residue at position 84, will be understood to indicate the amino acids that are at position 85 on the altered polypeptide. Likewise, a reference herein to substitution of a specific amino acid at a specific position, e.g., Y84, is understood to refer to a substitution of an amino acid for the amino acid at position 84 in the wild-type polypeptide. A Y84C substitution is thus understood to be a substitution of Cys residue for the Tyr residue that is present in the wild-type sequence. If, e.g., the wild-type polypeptide is altered to change the amino acid at position 84 from its wild-type amino acid to an alternate amino acid, then the substitution for the amino acid at position 84 will be understood to refer to the substitution for the alternate amino acid. If in such case the polypeptide is also altered by the addition or deletion of one or more amino acids, then the reference to the substitution will be understood to refer to the substitution for the alternate amino acid at the altered position number. A reference to a “non-naturally occurring Cys residue” in a polypeptide, e.g., an MHC class I polypeptide, means that the polypeptide comprises a Cys residue in a location where there is no Cys in the corresponding wild-type polypeptide. This can be accomplished through routine protein engineering in which a cysteine is substituted for the amino acid that occurs in the wild-type sequence.

A polynucleotide or polypeptide has a certain percent “sequence identity” to another polynucleotide or polypeptide, meaning that, when aligned, that percentage of bases or amino acids are the same, and in the same relative position, when comparing the two sequences. Sequence identity can be determined in a number of different ways. To determine sequence identity, sequences can be aligned using various convenient methods and computer programs (e.g., BLAST, T-COFFEE, MUSCLE, MAFFT, etc.), available over the world wide web at sites including ncbi.nlm.nili.gov/BLAST, ebi.ac.uk/Tools/msa/tcoffee/, ebi.ac.uk/Tools/msa/muscle/, mafft.cbrc.jp/alignment/software/. See, e.g., Altschul et al. (1990), J. Mol. Bioi. 215:403-10. Unless otherwise stated, “sequence identity” as referred to herein is determined by BLAST (Basic Local Alignment Search Tool), as described in Altschul et al. (1990) J. Mol. Biol. 215:403.

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide containing side chains consisting of asparagine and glutamine; a group of amino acids having aromatic side chains consists of phenylalanine, tyrosine, and tryptophan; a group of amino acids having basic side chains consists of lysine, arginine, and histidine; a group of amino acids having acidic side chains consists of glutamate and aspartate; and a group of amino acids having sulfur containing side chains consists of cysteine and methionine. Exemplary conservative amino acid substitution groups are: valine-leucine-isoleucine, phenylalanine-tyrosine, lysine-arginine, alanine-valine-glycine, and asparagine-glutamine.

The term “immunological synapse” or “immune synapse” as used herein generally refers to the natural interface between two interacting immune cells of an adaptive immune response including, e.g., the interface between an antigen-presenting cell (APC) or target cell and an effector cell, e.g., a lymphocyte, an effector T cell, a natural killer cell, and the like. An immunological synapse between an APC and a T cell is generally initiated by the interaction of a T cell antigen receptor and major histocompatibility complex molecules, e.g., as described in Bromley et al., Annu Rev Immunol. (2001) 19:375-96; the disclosure of which is incorporated herein by reference in its entirety.

“T cell” includes all types of immune cells expressing CD3, including T-helper cells (CD4⁺ cells), cytotoxic T-cells (CD8⁺ cells), T-regulatory cells (Treg), and NK-T cells.

The term “immunomodulatory polypeptide” (also referred to herein as a “MOD”), as used herein, means a polypeptide that specifically binds a cognate costimulatory polypeptide on a T cell, thereby providing a signal which, in addition to the primary signal provided by, for instance, binding of a TCR/CD3 complex with a major histocompatibility complex (MHC) polypeptide loaded with peptide, mediates a T cell response, including, but not limited to, proliferation, activation, differentiation, and the like. As discussed herein, a MOD can include, but is not limited to wild-type or variants of wild-type polypeptides such as a cytokine (e.g., IL-2), CD7, B7-1 (CD80), B7-2 (CD86), PD-L1, PD-L2, 4-1BBL, OX40L, Fas ligand (FasL), inducible costimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll ligand receptor, and a ligand that specifically binds with B7-H3. A MOD of a TMMP can bind a cognate costimulatory polypeptide (i.e., a “co-MOD”) that is present on a target T cell.

As used herein the term “in vivo” refers to any process or procedure occurring inside of the body.

As used herein, “in vitro” refers to any process or procedure occurring outside of the body.

“Heterologous,” as used herein, means a nucleotide or polypeptide that is not found in the native nucleic acid or protein, respectively.

“Recombinant,” as used herein, means that a particular nucleic acid (DNA or RNA) is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides, to provide a synthetic nucleic acid which is capable of being expressed from a recombinant transcriptional unit contained in a cell or in a cell-free transcription and translation system.

The terms “recombinant expression vector,” or “DNA construct” are used interchangeably herein to refer to a DNA molecule comprising a vector and at least one insert. Recombinant expression vectors are usually generated for the purpose of expressing and/or propagating the insert(s), or for the construction of other recombinant nucleotide sequences. The insert(s) may or may not be operably linked to a promoter sequence and may or may not be operably linked to DNA regulatory sequences.

As used herein, the term “affinity” refers to the equilibrium constant for the reversible binding of two agents (e.g., an antibody and an antigen) and is expressed as a dissociation constant (K_{D}). As used herein, the term “avidity” refers to the resistance of a complex of two or more agents to dissociation after dilution. The terms “immunoreactive” and “preferentially binds” are used interchangeably herein with respect to antibodies and/or antigen-binding fragments.

The term “binding,” as used herein (e.g., with reference to binding of a TMMP to a polypeptide (e.g., a T-cell receptor) on a T cell), refers to a non-covalent interaction between two molecules. Non-covalent binding refers to a direct association between two molecules, due to, for example, electrostatic, hydrophobic, ionic, and/or hydrogen-bond interactions, including interactions such as salt bridges and water bridges. “Affinity” refers to the strength of non-covalent binding, increased binding affinity being correlated with a lower K_{D}. “Specific binding” generally refers to binding of a ligand to a moiety that is than its designated binding site or receptor. “Non-specific binding” generally refers to binding of a ligand to a moiety other than its designated binding site or receptor. “Covalent binding” or “covalent bond,” as used herein, refers to the formation of one or more covalent chemical binds between two different molecules.

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease or symptom in a mammal, and includes: (a) preventing the disease or symptom from occurring in a subject which may or may not be predisposed to acquiring the disease or symptom but has not yet been diagnosed as having it; (b) inhibiting the disease or one or more symptoms associated with the disease, e.g., arresting its development; and/or (c) relieving the disease, i.e., causing regression of the disease. The therapeutic agent may be administered before, during and/or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease.

The terms “individual,” “subject,” “host,” and “patient,” are used interchangeably herein and refer to any mammalian subject for whom diagnosis, treatment, or therapy is desired. Mammals include, e.g., humans, non-human primates, rodents (e.g., rats; mice), lagomorphs (e.g., rabbits), ungulates (e.g., cows, sheep, pigs, horses, goats, and the like), etc. Unless otherwise indicated, the terms “individual,” “subject,” “host,” and “patient,” refer to a human.

The terms “antibodies” and “immunoglobulin” include antibodies or immunoglobulins of any isotype, fragments of antibodies that retain specific binding to antigen, including, but not limited to, Fab, Fv, scFv, and Fd fragments, chimeric antibodies, humanized antibodies, single-chain antibodies (scAb), single domain antibodies (dAb), single domain heavy chain antibodies, a single domain light chain antibodies, nanobodies, bi-specific antibodies, multi-specific antibodies, and fusion proteins comprising an antigen-binding (also referred to herein as antigen binding) portion of an antibody and a non-antibody protein. The antibodies can be detectably labeled, e.g., with a radioisotope, an enzyme that generates a detectable product, a fluorescent protein, and the like. The antibodies can be further conjugated to other moieties, such as members of specific binding pairs, e.g., biotin (member of biotin-avidin specific binding pair), and the like. Also encompassed by the term are Fab′, Fv, F(ab′)₂, and or other antibody fragments that retain specific binding to antigen, and monoclonal antibodies. As used herein, a monoclonal antibody is an antibody produced by a group of identical cells, all of which were produced from a single cell by repetitive cellular replication. That is, the clone of cells only produces a single antibody species. While a monoclonal antibody can be produced using hybridoma production technology, other production methods known to those skilled in the art can also be used (e.g., antibodies derived from antibody phage display libraries). An antibody can be monovalent or bivalent. An antibody can be an Ig monomer, which is a “Y-shaped” molecule that consists of four polypeptide chains: two heavy chains and two light chains connected by disulfide bonds.

The term “nanobody” (Nb), as used herein, refers to the smallest antigen binding fragment or single variable domain (V_{HH}) derived from naturally occurring heavy chain antibody and is known to the person skilled in the art. They are derived from heavy chain only antibodies, seen in camelids (Hamers-Casterman et al. (1993) Nature 363:446; Desmyter et al. (1996) Nature Structural Biol. 3:803; and Desmyter et al. (2015) Curr. Opin. Struct. Biol. 32:1). In the family of “camelids” immunoglobulins devoid of light polypeptide chains are found. “Camelids” comprise old world camelids (Camelus bactrianus and Camelus dromedarius) and new world camelids (for example, Llama paccos, Llama glama, Llama guanicoe and Llama vicugna). A single variable domain heavy chain antibody is referred to herein as a nanobody or a V_{HH} antibody.

“Antibody fragments” comprise a portion of an intact antibody, for example, the antigen binding or variable region of the intact antibody. Examples of antibody fragments include Fab, Fab′, F(ab′)₂, and Fv fragments; diabodies; linear antibodies (Zapata et al., Protein Eng. 8(10): 1057-1062 (1995)); domain antibodies (dAb; Holt et al. (2003) Trends Biotechnol. 21:484); single-chain antibody molecules; and multi-specific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)₂ fragment that has two antigen combining sites and is still capable of cross-linking antigen.

“Fv” is the minimum antibody fragment that contains a complete antigen-recognition and -binding site. This region consists of a dimer of one heavy- and one light-chain variable domain in tight, non-covalent association. It is in this configuration that the three CDRS of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The “Fab” fragment also contains the constant domain of the light chain and the first constant domain (CH1) of the heavy chain. Fab fragments differ from Fab′ fragments by the addition of a few residues at the carboxyl terminus of the heavy chain CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG, and IgM, and several of these classes can be further divided into subclasses (isotypes), e.g., IgG1, IgG2, IgG3, IgG4, IgA, and IgA2. The subclasses can be further divided into types, e.g., IgG2a and IgG2b.

“Single-chain Fv” or “sFv” or “scFv” antibody fragments comprise the V_{H} and V_{L} domains of antibody, wherein these domains are present in a single polypeptide chain. In some embodiments, the Fv polypeptide further comprises a polypeptide linker between the VH and VL domains, which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (VH) connected to a light-chain variable domain (VL) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al. (1993) Proc. Natl. Acad. Sci. USA 90:6444-6448.

As used herein, the term “CDR” or “complementarity determining region” is intended to mean the non-contiguous antigen combining sites found within the variable region of both heavy and light chain polypeptides. CDRs have been described by Kabat et al (1977) J. Biol. Chem. 252:6609; Kabat et al., U.S. Dept. of Health and Human Services, “Sequences of proteins of immunological interest” (1991) (also referred to herein as Kabat 1991); by Chothia et al. (1987) J. Mol. Biol. 196:901 (also referred to herein as Chothia 1987); and MacCallum et al. (1996) J. Mol. Biol. 262:732, where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues, which encompass the CDRs, as defined by each of the above cited references are set forth below in Table 1 as a comparison.

TABLE 1
CDR Definitions

| | Kabat¹ | Chothia² | MacCallum³ |
|---|---|---|---|
| V_{H} CDR-1 | 31-35 | 26-32 | 30-35 |
| V_{H} CDR-2 | 50-65 | 53-55 | 47-58 |
| V_{H} CDR-3 | 95-102 | 96-101 | 93-101 |
| V_{L} CDR-1 | 24-34 | 26-32 | 30-36 |
| V_{L} CDR-2 | 50-56 | 50-52 | 46-55 |
| V_{L} CDR-3 | 89-97 | 91-96 | 89-96 |

¹Residue numbering follows the nomenclature of Kabat et al., 1991, supra
²Residue numbering follows the nomenclature of Chothia et al., supra
³Residue numbering follows the nomenclature of MacCallum et al., supra

As used herein, the terms “CDR-L1”, “CDR-L2”, and “CDR-L3” refer, respectively, to the first, second, and third CDRs in a light chain variable region. The terms “CDR-L1”, “CDR-L2”, and “CDR-L3” may be used interchangeably with “VL CDR1,” “VL CDR2,” and “VL CDR3,” respectively. As used herein, the terms “CDR-H1”, “CDR-H2”, and “CDR-H3” refer, respectively, to the first, second, and third CDRs in a heavy chain variable region. The terms “CDR-H1”, “CDR-H2”, and “CDR-H3” may be used interchangeably with “VH CDR1,” “VH CDR2,” and “VH CDR3,” respectively. As used herein, the terms “CDR-1”, “CDR-2”, and “CDR-3” refer, respectively, to the first, second and third CDRs of either chain's variable region.

As used herein, the term “framework,” when used in reference to an antibody variable region, is intended to mean all amino acid residues outside the CDR regions within the variable region of an antibody. A variable region framework is generally a discontinuous amino acid sequence between about 100-120 amino acids in length but is intended to reference only those amino acids outside of the CDRs. As used herein, the term “framework region” is intended to mean each domain of the framework that is separated by the CDRs.

Unless indicated otherwise, the term “substantially” is intended to encompass both “wholly” and “largely but not wholly”. For example, an Ig Fc that “substantially does not induce ADCC” means an Ig Fc that induces no ADCC at all or that largely does not induce ADCC.

As used herein, the term “about” used in connection with an amount indicates that the amount can vary by 10% of the stated amount. For example, “about 100” means an amount of from 90-110. Where about is used in the context of a range, the “about” used in reference to the lower amount of the range means that the lower amount includes an amount that is 10% lower than the lower amount of the range, and “about” used in reference to the higher amount of the range means that the higher amount includes an amount 10% higher than the higher amount of the range. For example, from about 100 to about 1000 means that the range extends from 90 to 1100.

As used herein, the term “MHC heavy chain polypeptide” means collectively the domains of an MHC heavy chain polypeptide that are present in a TMMP. For example, an MHC heavy chain polypeptide can comprise α1, α2 and α3 domains.

Before the present disclosure is further described, it is to be understood that this disclosure is not limited to particular embodiments described, as such may, of course, vary. It is also to be understood that the terminology used herein is for the purpose of describing particular embodiments only, and is not intended to be limiting, since the scope will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

As used herein and in the appended claims, the singular forms “a,” “an,” and “the” include plural referents unless the context clearly dictates otherwise. Thus, for example, reference to a “T-cell modulatory polypeptide” includes a plurality of such polypeptides and reference to “the immunomodulatory polypeptide” includes reference to one or more immunomodulatory polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

The publications discussed herein are provided solely for their disclosure. Nothing herein is to be construed as an admission that the present disclosure is not entitled to antedate such publication. Further, the dates of publication provided may be different from the actual publication dates which may need to be independently confirmed.

### DETAILED DESCRIPTION

The present disclosure provides T-cell modulatory multimeric polypeptides (TMMPs) that comprise (i) an optional immunomodulatory polypeptide such as a variant IL-2 polypeptide, (ii) class I HLA major histocompatibility complex (MHC) polypeptides (a class I HLA heavy chain polypeptide and a f2 microglobulin polypeptide), (iii) a peptide that presents an epitope to a T-cell receptor, which together with the class I MHC polypeptides forms a peptide-MHC complex (pMHC), (iv) a tumor-targeting polypeptide, and (v) an optional Ig Fc polypeptide or other scaffold.

A TMMP is useful for modulating the activity of a T cell, and for modulating an immune response in an individual. Importantly, the TMMPs disclosed herein are useful for “redirecting” a patient's repertoire of anti-pathogenic (e.g., antiviral) T cells to attack and kill cancer cells. The TMMPs achieve this result by (i) binding to the patient's cancer cells via the TMMP's tumor-targeting polypeptide (TTP) that is specific for and binds an antigen on the cancer cell, and (ii) presenting a pMHC that mimics a pMHC on the surface of a pathogen-infected cell, e.g., a cell infected by a virus, bacteria or other microorganism that can cause disease. In this way, TMMPs effectively “paint” the cancer cells to make them appear like pathogen-infected cells. The “painted” cancer cells are then susceptible to being attacked and killed by the patient's existing repertoire of anti-pathogenic T cells that have a T cell receptor (TCR) that recognizes and binds to the pMHC presented by the TMMP.

For example, TMMPs can have a TTP that binds to an antigen on a cancer cell, and a pMHC that mimics the pMHC of a cell that has been infected with a virus such as a SARS-CoV-2 virus. The patient's existing repertoire of T cells against a SARS-CoV-2 virus-infected cells are thus re-directed to the patient's cancer cells where the T cells can be activated through binding of the TCR of T cells to the pMHC of the TMMP. Activation of the T cells can lead to release of their cytotoxic components and killing of the cancer cells. In such cases, the patient's repertoire of T cells specific the SARS-CoV-2 virus can be the result of one or more of a prior SARS-CoV-2 infection, vaccination with a vaccine that elicits T cells having a TCR that is specific for the pMHC of the TMMP, and/or prior treatment with a T cell modulatory protein designed to prime and/or increase the patient's T cells that have a TCR that specifically binds the pMHC of the TMMP.

The TMMPs disclosed herein are useful for any patient having a hematological cancer or solid tumor, including patients whose cancers have evaded their immune system through HLA loss, which prevents the patient's own cancer-specific T cells from recognizing and killing the cancer cells.

### T-Cell Modulatory Multimeric Polypeptides

The present disclosure provides a T-cell modulatory multimeric polypeptide (TMMP) comprising: a) a first polypeptide; and b) a second polypeptide, wherein the TMMP comprises: a peptide epitope (defined below); a first major histocompatibility complex (MHC) polypeptide; a second MHC polypeptide; one or more immunomodulatory polypeptides; an immunoglobulin (Ig) Fc polypeptide or a non-Ig scaffold; and a tumor targeting polypeptide (TTP).

In some cases, a TMMP comprises a heterodimer (comprises two different separate polypeptide chains) comprising: a) a first polypeptide; and b) a second polypeptide, wherein the TMMP comprises: a peptide epitope; a first MHC polypeptide; a second MHC polypeptide; one or more immunomodulatory polypeptides; an Ig Fc polypeptide or a non-Ig scaffold; and a TTP. A TMMP can comprise a single heterodimer. A TMMP can comprise two copies of a heterodimer; i.e., a TMMP can be a homodimer of a heterodimer. The two copies of a heterodimer can be linked to one another, e.g., by disulfide bonds (e.g., disulfide bonds between Ig Fc polypeptides present in the heterodimers)

In some cases, a TMMP comprises one or more heterodimers, each heterodimer comprising: a) a first polypeptide comprising a first MHC polypeptide; and b) a second polypeptide comprising a second MHC polypeptide, wherein the first polypeptide or the second polypeptide comprises a peptide epitope (defined below), wherein the first polypeptide and/or the second polypeptide comprises one or more immunomodulatory polypeptides that can be the same or different; and an Ig Fc polypeptide or a non-Ig scaffold. The first or the second polypeptide also includes a TTP. In some cases, the TTP is at the C-terminus of the Ig Fc polypeptide or the non-Ig scaffold. In some cases, the TTP is at the N-terminus of the polypeptide that comprises the Ig Fc polypeptide. In some cases, the TTP is at the C-terminus of the polypeptide that comprises the peptide epitope.

The peptide epitope present in a TMMP presents a SARS-CoV-2 peptide (e.g., a SARS-CoV-2 encoded peptide). As used herein, the term “peptide epitope” means a peptide that, when complexed with MHC polypeptides, presents an epitope to a T-cell receptor (TCR). When complexed with MHC polypeptides, a peptide epitope can present one or more epitopes to one or more TCRs.

In some cases, a TMMP includes: i) a SARS-CoV-2 peptide; and iii) a TTP that targets a cancer-associated antigen. Such a TMMP binds a cancer cell that expresses the cancer-associated antigen targeted by the TTP. The TMMP modulates the activity of a T-cell specific for the virus epitope present in the TMMP. For example, in some cases, the TMMP increases proliferation and/or cytotoxic activity of a T-cell specific for the virus epitope present in the TMMP. Contacting a T-cell specific for the virus epitope present in the TMMP can increase cytotoxic activity of the T cell toward a cancer cell expressing the cancer-associated antigen that is targeted by the TTP present in the TMMP.

TMMPs comprising a pMHC that has a SARS-CoV-2 epitope are particularly useful because the high rate of SARS-CoV-2 infections worldwide and the availability of commercial vaccines means that most patients either already have, or can be induced to have through vaccination, a repertoire of T cells that can recognize a pMHC having a SARS-CoV-2 epitope. As noted above, however, a pMHC of a TMMP alternatively could present a peptide epitope from other virus or pathogenic microorganisms against which the patient has an existing repertoire of T cells. For example, the pMHC could present an epitope that would be recognized by T cells that result from administration of commonly available vaccines that provide immunity against pathogens such as tetanus, flu or other available vaccines. There are numerous commercially available vaccines against pathogens, e.g., Cholera, Dengue, Diphtheria, Hepatitis, Haemophilus influenzae type b (Hib), Human papillomavirus (HPV), Influenza, Japanese encephalitis, Malaria, Measles, Meningococcal meningitis, Mumps, Pertussis, Pneumococcal disease, Poliomyelitis, Rabies, Rotavirus, Rubella, Tetanus, Tick-borne encephalitis, Tuberculosis, Typhoid, Varicella and Yellow Fever, with numerous other in development, e.g., Enterotoxigenic Escherichia coli, Group B Streptococcus (GBS), Herpes Simplex Virus, HIV-1, Malaria, Neisseria gonorrhoeae, Nontyphoidal Salmonella Disease, Norovirus, Paratyphoid fever, Respiratory Syncytial Virus (RSV), Schistosomiasis Disease, Shigella, Group A Streptococcus (GAS), Tuberculosis, and other Influenza vaccines. Alternatively, (or additionally), prior to administering the TMMP, one or more doses of a T cell modulatory polypeptide can be administered to the patient, in order to prime and activate and/or induce proliferation of (and thereby increase the number of) CD8⁺ T cells that recognize the desired pMHC in the TMMP comprising the TTP. See, e.g., the T cell modulatory polypeptides described in WO2021/195108, published Sep. 30, 2021 (Cue Biopharma, Inc.), WO 2021/195411, published Sep. 30, 2021 (Cue Biopharma, Inc.), and WO 2022/015880, published Jan. 20, 2022 (Cue Biopharma, Inc.).

In some cases, a TMMP comprises one or more heterodimers, each heterodimer comprising: a) a first polypeptide comprising: i) a peptide epitope (a SARS-CoV-2 peptide); and ii) a first MHC polypeptide; b) a second polypeptide comprising a second MHC polypeptide; c) one or more immunomodulatory polypeptides, where the first and/or the second polypeptide comprises the at least one (i.e., one or more) immunomodulatory polypeptide; d) an Ig Fc polypeptide or a non-Ig scaffold, where the first and/or the second polypeptide comprises the Ig Fc polypeptide or the non-Ig scaffold; and e) a polypeptide that targets a cancer cell (a tumor-targeting polypeptide; “TTP”). In some cases, at least one of the one or more immunomodulatory polypeptides comprises an amino acid sequence of a wild-type immunomodulatory polypeptide. In some cases, at least one of the one or more immunomodulatory polypeptides is a variant immunomodulatory polypeptide that binds to but exhibits reduced affinity to a cognate co-immunomodulatory polypeptide compared to the affinity of a corresponding wild-type immunomodulatory polypeptide for the cognate co-immunomodulatory polypeptide. In some cases, the peptide epitope present in a TMMP presents an infectious disease-associated epitope (e.g., a virus-encoded peptide).

A TMMP comprises a tumor targeting polypeptide, i.e., a polypeptide that targets a cancer-associated epitope displayed on the surface of a cancer cell.

In some cases, a TMMP is:
A) a heterodimer comprising: a) a first polypeptide comprising a first MHC polypeptide; and b) a second polypeptide comprising a second MHC polypeptide, wherein the first polypeptide or the second polypeptide comprises a SARS-CoV-2 peptide, wherein the first polypeptide and/or the second polypeptide comprises one or more immunomodulatory polypeptides that can be the same or different, and wherein at least one of the one or more immunomodulatory polypeptides may be a wild-type immunomodulatory polypeptide or a variant of a wild-type immunomodulatory polypeptide; wherein the first polypeptide and/or the second polypeptide comprises an Ig Fc polypeptide or a non-Ig scaffold; and wherein the first and/or the second polypeptide comprises a TTP; or
B) a heterodimer comprising: a) a first polypeptide comprising a first MHC polypeptide; and b) a second polypeptide comprising a second MHC polypeptide, wherein the first polypeptide or the second polypeptide comprises a SARS-CoV-2 peptide; wherein the first polypeptide and/or the second polypeptide comprises one or more immunomodulatory polypeptides that can be the same or different,
wherein at least one of the one or more immunomodulatory polypeptides is a variant of a wild-type immunomodulatory polypeptide,
wherein at least one of the one or more immunomodulatory polypeptides is a variant immunomodulatory polypeptide that exhibits reduced affinity to a cognate co-immunomodulatory polypeptide compared to the affinity of a corresponding wild-type immunomodulatory polypeptide for the cognate co-immunomodulatory polypeptide.; and
wherein the first polypeptide and/or the second polypeptide comprises an Ig Fc polypeptide or a non-Ig scaffold; and wherein the first and/or the second polypeptide comprises a TTP; or
C) a heterodimer comprising: a) a first polypeptide comprising, in order from N-terminus to C-terminus: i) a SARS-CoV-2 peptide; ii) a first MHC polypeptide; and b) a second polypeptide comprising, in order from N-terminus to C-terminus: i) a second MHC polypeptide; and ii) optionally an immunoglobulin (Ig) Fc polypeptide or a non-Ig scaffold, wherein the TMMP comprises one or more immunomodulatory polypeptides that can be the same or different, wherein at least one of the one or more immunomodulatory polypeptides is: A) at the C-terminus of the first polypeptide; B) at the N-terminus of the second polypeptide; C) at the C-terminus of the second polypeptide; or D) at the C-terminus of the first polypeptide and at the N-terminus of the second polypeptide, and wherein at least one of the one or more immunomodulatory polypeptides may be a wild-type immunomodulatory polypeptide or a variant of a wild-type immunomodulatory polypeptide; and
optionally wherein at least one of the one or more immunomodulatory polypeptides is a variant immunomodulatory polypeptide that exhibits reduced affinity to a cognate co-immunomodulatory polypeptide compared to the affinity of a corresponding wild-type immunomodulatory polypeptide for the cognate co-immunomodulatory polypeptide.

In some cases, an immunomodulatory polypeptide present in a TMMP comprises a wild-type (naturally-occurring) amino acid sequence.

In some cases, an immunomodulatory polypeptide present in a TMMP binds to its cognate co-immunomodulatory polypeptide with an affinity that is less than the affinity of a corresponding wild-type immunomodulatory polypeptide for the cognate co-immunomodulatory polypeptide.

The combination of the reduced affinity of the immunomodulatory polypeptide for its cognate co-immunomodulatory polypeptide, and the affinity of the epitope for a TCR, provides for enhanced selectivity of a TMMP. For example, a TMMP binds selectively to a first T cell that displays both: i) a TCR specific for the epitope present in the TMMP; and ii) a co-immunomodulatory polypeptide that binds to the immunomodulatory polypeptide present in the TMMP, compared to binding to a second T cell that displays: i) a TCR specific for an epitope other than the epitope present in the TMMP; and ii) a co-immunomodulatory polypeptide that binds to the immunomodulatory polypeptide present in the TMMP.

In some cases, a TMMP, when administered to an individual in need thereof, induces both an epitope-specific T cell response and an epitope non-specific T cell response. In other words, in some cases, a TMMP, when administered to an individual in need thereof, induces an epitope-specific T cell response by modulating the activity of a first T cell that displays both: i) a TCR specific for the peptide epitope present in the TMMP; ii) a co-immunomodulatory polypeptide that binds to the immunomodulatory polypeptide present in the TMMP; and induces an epitope non-specific T cell response by modulating the activity of a second T cell that displays: i) a TCR specific for an epitope other than the epitope present in the TMMP; and ii) a co-immunomodulatory polypeptide that binds to the immunomodulatory polypeptide present in the TMMP. The ratio of the epitope-specific T cell response to the epitope-non-specific T cell response is at least 2:1, at least 5:1, at least 10:1, at least 15:1, at least 20:1, at least 25:1, at least 50:1, or at least 100:1. The ratio of the epitope-specific T cell response to the epitope-non-specific T cell response is from about 2:1 to about 5:1, from about 5:1 to about 10:1, from about 10:1 to about 15:1, from about 15:1 to about 20:1, from about 20:1 to about 25:1, from about 25:1 to about 50:1, or from about 50:1 to about 100:1, or more than 100:1. “Modulating the activity” of a T cell can include one or more of: i) activating a cytotoxic (e.g., CD8⁺) T cell; ii) inducing cytotoxic activity of a cytotoxic (e.g., CD8⁺) T cell; iii) inducing production and release of a cytotoxin (e.g., a perforin; a granzyme; a granulysin) by a cytotoxic (e.g., CD8⁺) T cell; iv) inhibiting activity of an autoreactive T cell; and the like. As used herein, selectively modulating refers to inducing an epitope-specific T cell response that is greater than the epitope-non-specific T cell response.

The combination of the reduced affinity of the immunomodulatory polypeptide for its cognate co-immunomodulatory polypeptide, and the affinity of the epitope for a TCR, provides for enhanced selectivity of a TMMP. Thus, for example, a TMMP binds with higher avidity to a first T cell that displays both: i) a TCR specific for the epitope present in the TMMP; and ii) a co-immunomodulatory polypeptide that binds to the immunomodulatory polypeptide present in the TMMP, compared to the avidity to which it binds to a second T cell that displays: i) a TCR specific for an epitope other than the epitope present in the TMMP; and ii) a co-immunomodulatory polypeptide that binds to the immunomodulatory polypeptide present in the TMMP.

Binding affinity between an immunomodulatory polypeptide and its cognate co-immunomodulatory polypeptide can be determined by bio-layer interferometry (BLI) as described in published PCT Application WO 2020/243315 (Cue Biopharma, Inc.).

### Dimerized TMMPs

A TMMP can be dimerized; i.e., the present disclosure provides a multimeric polypeptide comprising a dimer of a TMMP. Thus, the present disclosure provides a TMMP comprising: A) a first heterodimer comprising: a) a first polypeptide comprising: i) a peptide epitope; and ii) a first major histocompatibility complex (MHC) polypeptide; and b) a second polypeptide comprising: i) a second MHC polypeptide, wherein the first heterodimer comprises one or more immunomodulatory polypeptides; and B) a second heterodimer comprising: a) a first polypeptide comprising: i) a peptide epitope; and ii) a first MHC polypeptide; and b) a second polypeptide comprising: i) a second MHC polypeptide, wherein the second heterodimer comprises one or more immunomodulatory polypeptides, and wherein the first heterodimer and the second heterodimer are covalently linked to one another. In some cases, the two TMMPs are identical to one another in amino acid sequence. In some cases, the first heterodimer and the second heterodimer are covalently linked to one another via a C-terminal region of the second polypeptide of the first heterodimer and a C-terminal region of the second polypeptide of the second heterodimer. In some cases, first heterodimer and the second heterodimer are covalently linked to one another via the C-terminal amino acid of the second polypeptide of the first heterodimer and the C-terminal region of the second polypeptide of the second heterodimer; for example, in some cases, the C-terminal amino acid of the second polypeptide of the first heterodimer and the C-terminal region of the second polypeptide of the second heterodimer are linked to one another, either directly or via a linker. The linker can be a peptide linker. The peptide linker can have a length of from 1 amino acid to 200 amino acids (e.g., from 1 amino acid (aa) to 5 aa, from 5 aa to 10 aa, from 10 aa to 25 aa, from 25 aa to 50 aa, from 50 aa to 100 aa, from 100 aa to 150 aa, or from 150 aa to 200 aa). In some cases, the peptide epitope of the first heterodimer and the peptide epitope of the second heterodimer comprise the same amino acid sequence. In some cases, the first MHC polypeptide of the first and the second heterodimer is an MHC Class I β2-microglobulin, and wherein the second MHC polypeptide of the first and the second heterodimer is an MHC Class I heavy chain. In some cases, the immunomodulatory polypeptide of the first heterodimer and the immunomodulatory polypeptide of the second heterodimer comprise the same amino acid sequence. In some cases, the immunomodulatory polypeptide of the first heterodimer and the immunomodulatory polypeptide of the second heterodimer are variant immunomodulatory polypeptides that comprise from 1 to 10 amino acid substitutions compared to a corresponding parental wild-type immunomodulatory polypeptide, and wherein the from 1 to 10 amino acid substitutions result in reduced affinity binding of the variant immunomodulatory polypeptide to a cognate co-immunomodulatory polypeptide. In some cases, the immunomodulatory polypeptide of the first heterodimer and the immunomodulatory polypeptide of the second heterodimer are each independently selected from the group consisting of IL-2, 4-1BBL, PD-L1, CD80, CD86, ICOS-L, OX-40L, FasL, JAG1 (CD339), TGFβ, CD70, and ICAM. Examples, of suitable MHC polypeptides, immunomodulatory polypeptides, and peptide epitopes are described below. The first and/or the second polypeptide comprises: i) an Ig Fc polypeptide or a non-Ig scaffold; and ii) a tumor-targeting polypeptide.

### Multiple Disulfide Bonded TMMPs

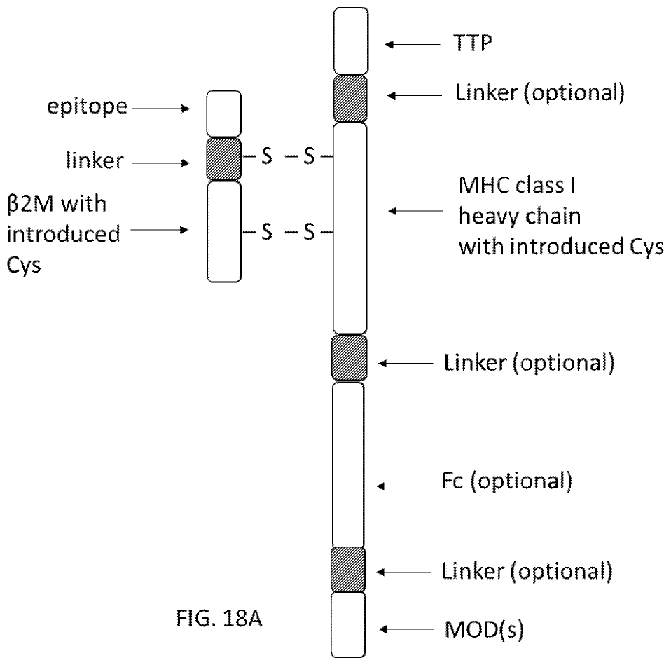

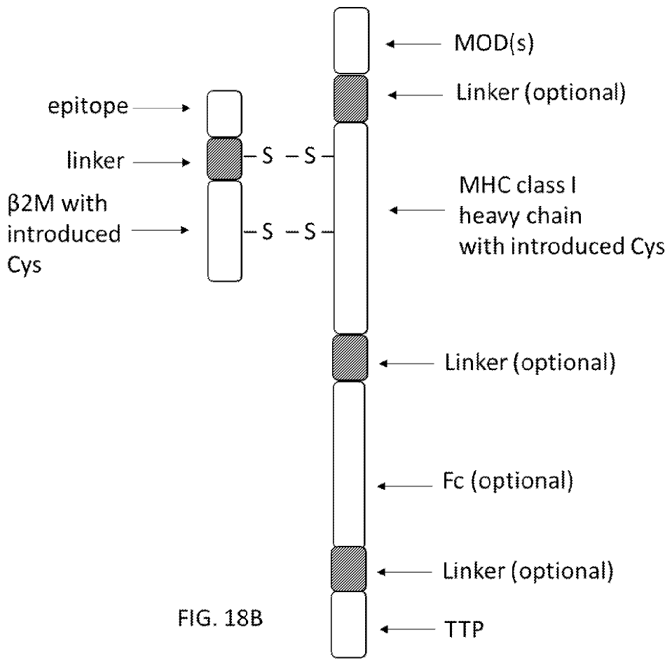

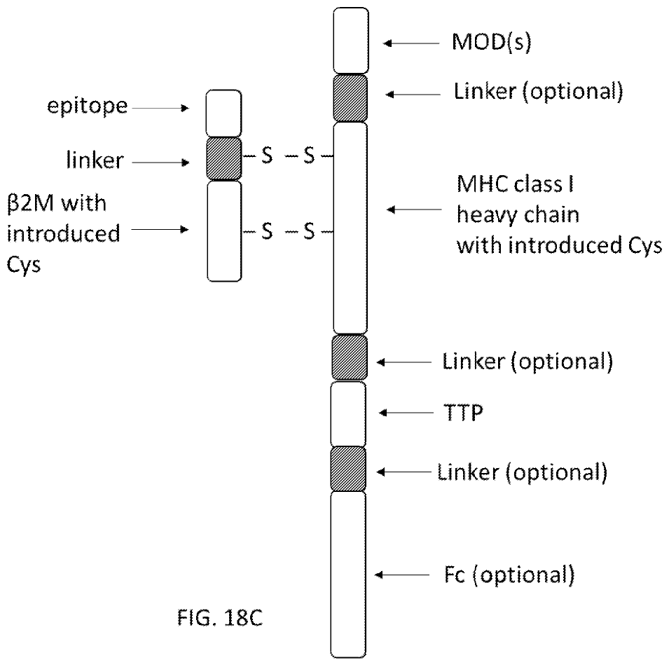

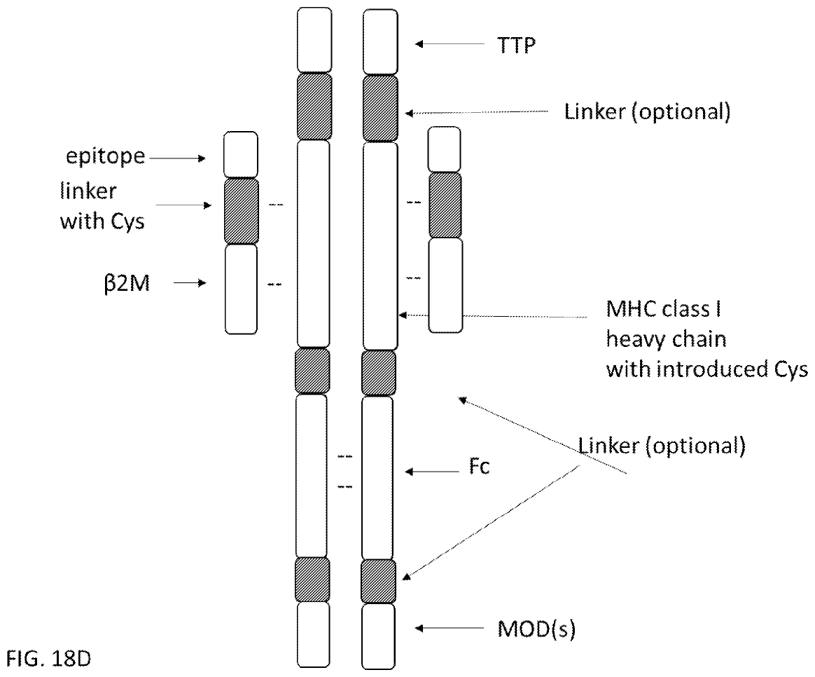

In some cases, the first polypeptide and the second polypeptide of a TMMP are linked to one another by at least two disulfide bonds (i.e., two interchain disulfide bonds). Examples of such multiple disulfide-linked TMMP are depicted schematically in FIG. 18A-FIG. 18C. In addition, where a TMMP comprises an IgFc polypeptide, a heterodimeric TMMP can be dimerized, such that disulfide bonds link the IgFc polypeptides in the two heterodimeric TMMPs. Such an arrangement is depicted schematically in FIG. 181), where disulfide bonds are represented by dashed lines. Unless otherwise stated, the at least two disulfide bonds described in the multiple disulfide-linked TMMPs in this section are not referring to disulfide bonds linking IgFc polypeptides in dimerized TMMPs.

As noted above, in some cases, the first polypeptide and the second polypeptide of a TMMMP are linked to one another by at least two disulfide bonds (i.e., two interchain disulfide bonds). For example, in some instances, the first polypeptide and the second polypeptide of a TMMP are linked to one another by 2 interchain disulfide bonds. As another example, in some instances, the first polypeptide and the second polypeptide of a TMMP are linked to one another by 3 interchain disulfide bonds. As another example, in some instances, the first polypeptide and the second polypeptide of a TMMP are linked to one another by 4 interchain disulfide bonds.

In some cases where a peptide epitope in a first polypeptide of a TMMP is linked to a 32M polypeptide by a linker comprising a Cys, at least one of the at least two disulfide bonds links a Cys in the linker to a Cys in an MHC Class I heavy chain in the second polypeptide. In some cases, where a peptide epitope in a first polypeptide of a TMMP is linked to an MHC Class I heavy chain polypeptide by a linker, at least one of the at least two disulfide bonds links a Cys in the linker to a Cys in a β2M polypeptide present in the second polypeptide.

In some cases, a T-cell modulatory multimeric polypeptide comprises at least one heterodimer comprising: a) a first polypeptide comprising: i) a peptide epitope; and ii) first MHC polypeptide; b) a second polypeptide comprising a second MHC polypeptide; c) at least one immunomodulatory polypeptide, where the first and/or the second polypeptide comprises the at least one immunomodulatory polypeptide; d) an Ig Fc polypeptide or a non-Ig scaffold, where the first and/or the second polypeptide comprises the Ig Fc polypeptide or the non-Ig scaffold; and e) a TTP, where the first and/or the second polypeptide comprises the TTP; and where the heterodimer comprises at least two disulfide bonds (e.g., two disulfide bonds) between the first polypeptide and the second polypeptide (e.g., the heterodimer comprises: i) a first disulfide bond linking the first polypeptide and the second polypeptide; and ii) a second disulfide bond linking the first polypeptide and the second polypeptide). Expressed another way, the first polypeptide comprises a first Cys residue that forms a disulfide bond (a first disulfide bond) with a first Cys residue in the second polypeptide; and the first polypeptide comprises a second Cys residue that forms a disulfide bond (a second disulfide bond) with a second Cys residue in the second polypeptide.

In some cases, a TMMP comprises: a) a first polypeptide comprising, in order from N-terminus to C-terminus: i) a peptide epitope; ii) a peptide linker; and iii) a β2M polypeptide; and b) a second polypeptide comprising an MIHC Class I heavy chain polypeptide, where one or both of the first and the second polypeptides comprises at least one immunomodulatory polypeptide, where the TMMP comprises: a) a first disulfide linkage between: i) a Cys present in the linker between the peptide epitope and the β2M polypeptide; and ii) a first Cys introduced into the MHC Class I heavy chain polypeptide; and b) at least a second disulfide linkage between the first polypeptide and the second polypeptide, where the at least a second disulfide linkage is between: i) a Cys in the first polypeptide that is C-terminal to the Cys present in the linker; and ii) a Cys in the second polypeptide that is C-terminal to the first Cys introduced into the MHC Class I heavy chain polypeptide. As noted above, the TMMP also includes: i) an Ig Fe polypeptide or a non-Ig scaffold; and ii) a TTP.

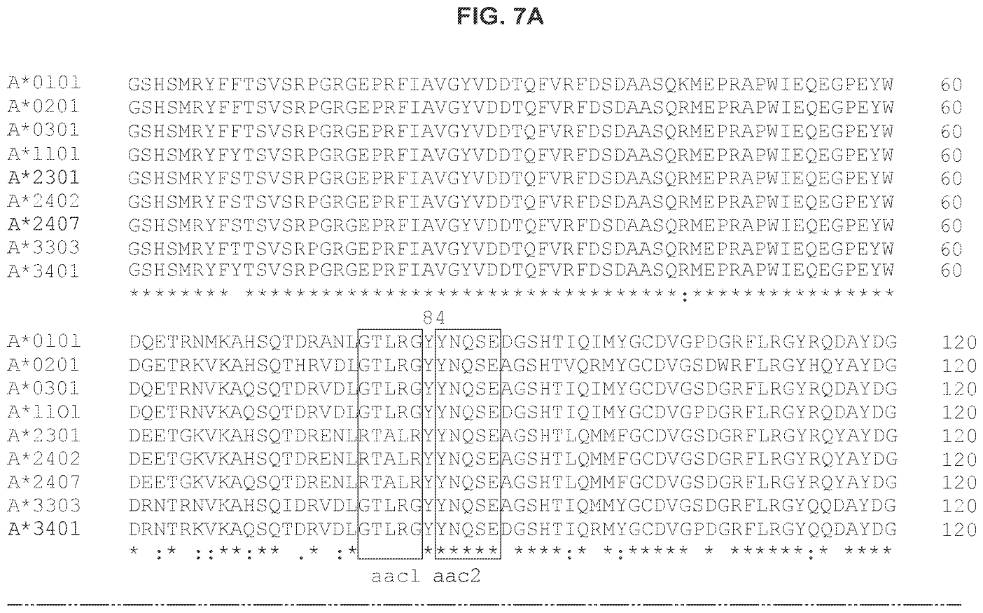

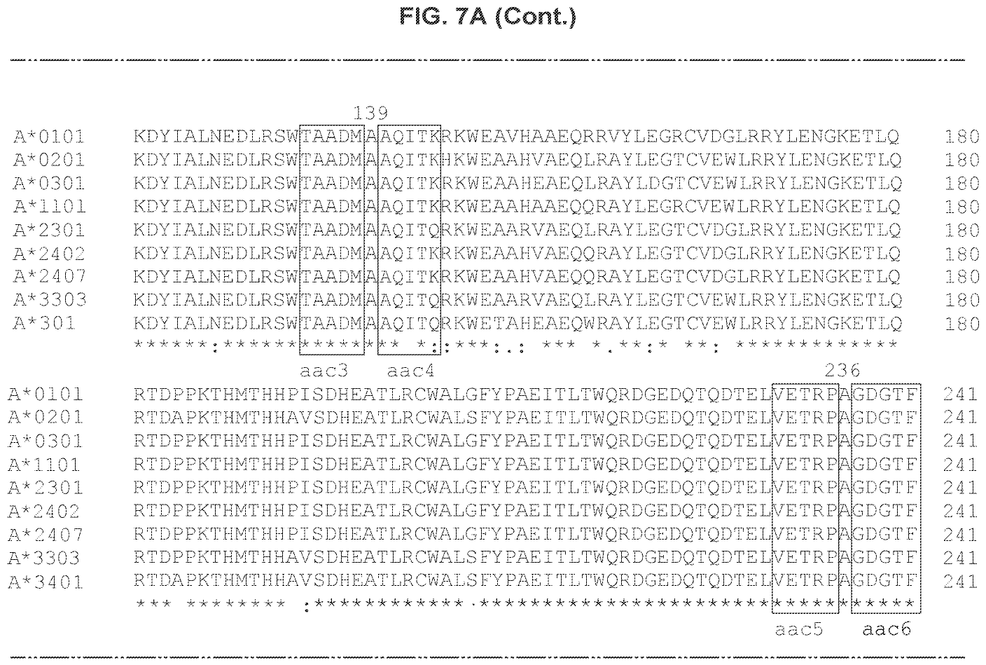

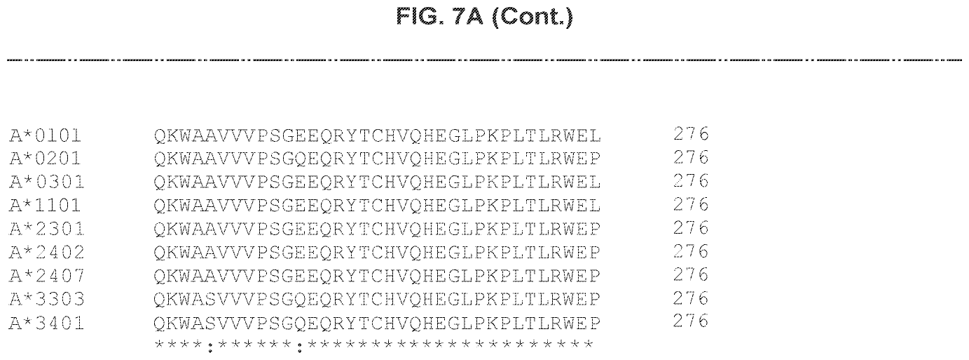

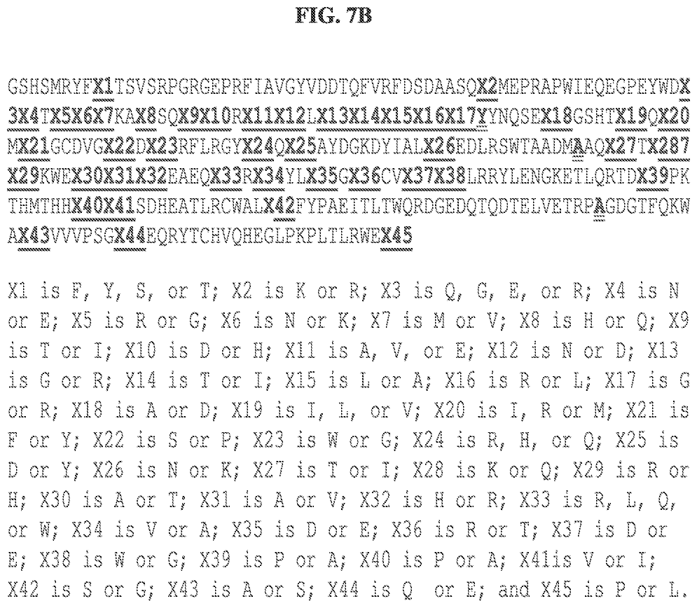

A multiple disulfide-linked TMMP (e.g., a double disulfide-linked TMMP) can comprise, for example: a) a first polypeptide comprising: i) a peptide epitope; and ii) a first MHC polypeptide, where the first polypeptide comprises a peptide linker between the peptide and the first MHC polypeptide, where the peptide linker comprises a Cys residue, and where the first MHC polypeptide is a β2M polypeptide that comprises an amino acid substitution that introduces a Cys residue; b) and a second polypeptide comprising a second MHC polypeptide, where the second MHC polypeptide is a Class I heavy chain comprising a Y84C substitution and an A236C substitution, based on the amino acid numbering of HLA-A*0201 (depicted in FIG. 7A), or at corresponding positions in another Class I heavy chain allele, where the TMMP comprises a disulfide bond between the Cys residue in the peptide linker and the Cys residue at amino acid position 84 of the Class I heavy chain or corresponding position of another Class I heavy chain allele, and where the TMMP comprises a disulfide bond between the introduced Cys residue in the β2M polypeptide and the Cys at amino acid position 236 of the Class I heavy chain or corresponding position of another Class I heavy chain allele; and c) at least one immunomodulatory polypeptide, where the first and/or the second polypeptide comprises the at least one immunomodulatory polypeptide. As noted above, the TMMP also includes: i) an Ig Fe polypeptide or a non-Ig scaffold; and ii) a TTP.

### Sars-Cov-2 Peptides

As discussed above, a TMMP comprises a Betacoronavirus (e.g., SARS-CoV-2) peptide that is typically at least about 4 amino acids in length, and presents a SARS-CoV-2 epitope to a T cell when in an MHC/peptide complex (e.g., an HLA/peptide complex).

A SARS-CoV-2 peptide present in a TMMP can have a length of at least 4 amino acids, e.g., from 4 amino acids to about 25 amino acids in length (e.g., 4 amino acids (aa), 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa, including within a range of from 4 to 20 amino acids, from 6 to 18 amino acids, from 8 to 15 amino acids, from 8 to 12 amino acids, from 9-11 amino acids, from 9-10 amino acids, from 5 to 10 amino acids, from 10-15 amino acids, from 10 to 20 amino acids, and from 15 to 25 amino acids in length), for example, lengths of 9, 10, 11, 12, 13 or 14 amino acids.

A SARS-CoV-2 epitope present in a TMMP is a peptide specifically bound by a T-cell, i.e., the epitope is specifically bound by an epitope-specific T cell. An epitope-specific T cell binds an epitope having a reference amino acid sequence, but does not substantially bind an epitope that differs from the reference amino acid sequence. For example, an epitope-specific T cell binds an epitope having a reference amino acid sequence, and binds an epitope that differs from the reference amino acid sequence, if at all, with an affinity that is less than 10⁻⁶ M, less than 10⁻⁵ M, or less than 10⁻⁴ M. An epitope-specific T cell can bind an epitope for which it is specific with an affinity of at least 10⁻⁷ M, at least 10⁻⁸ M, at least 10⁻⁹ M, or at least 10⁻¹⁰.

The peptide epitope present in a TMMP is a peptide of a Betacoronavirus-encoded polypeptide. In some cases, the peptide epitope is a SARS-CoV-2 peptide (i.e., a peptide of a SARS-CoV-2-encoded polypeptide). In some cases, the peptide epitope is a SARS-CoV-2 peptide from a SARS-CoV-2-encoded surface glycoprotein. In some cases, the peptide epitope is a SARS-CoV-2 peptide from a SARS-CoV-2-encoded membrane glycoprotein. In some cases, the peptide epitope is a SARS-CoV-2 peptide from a SARS-CoV-2-encoded nucleocapsid phosphoprotein.

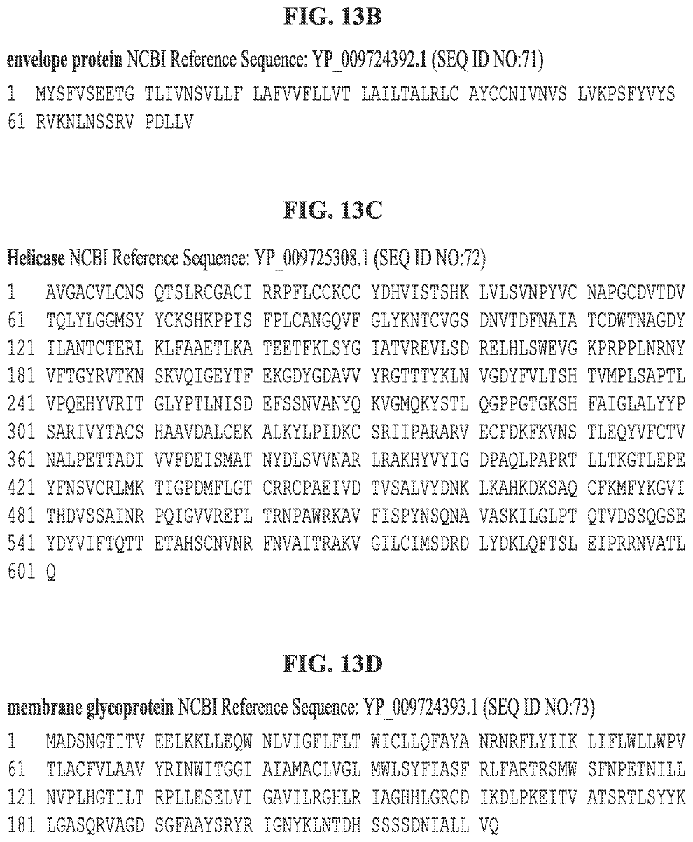

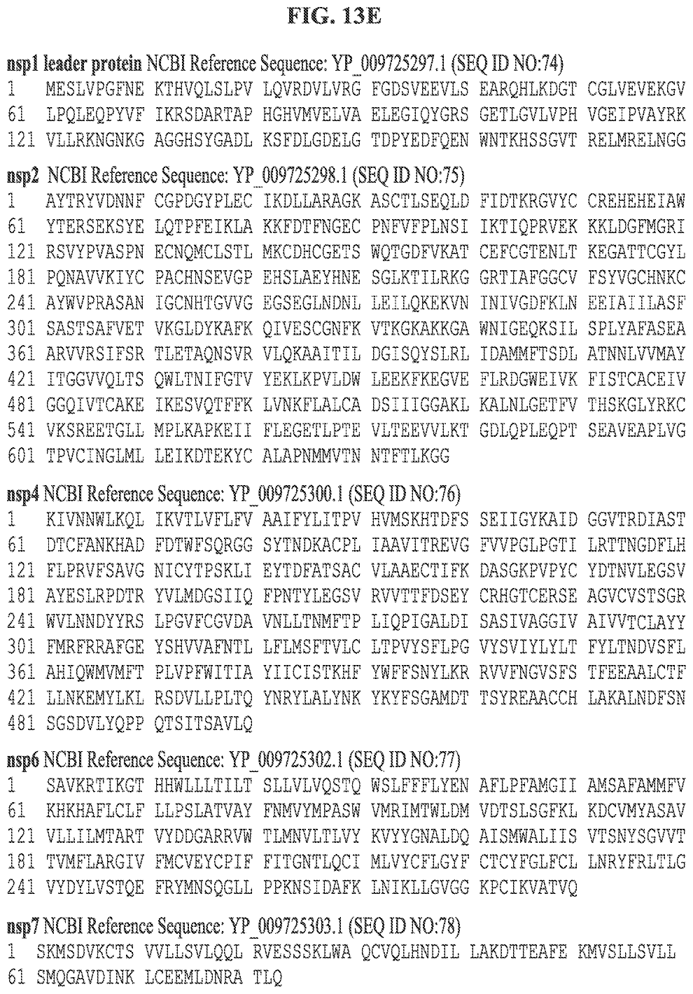

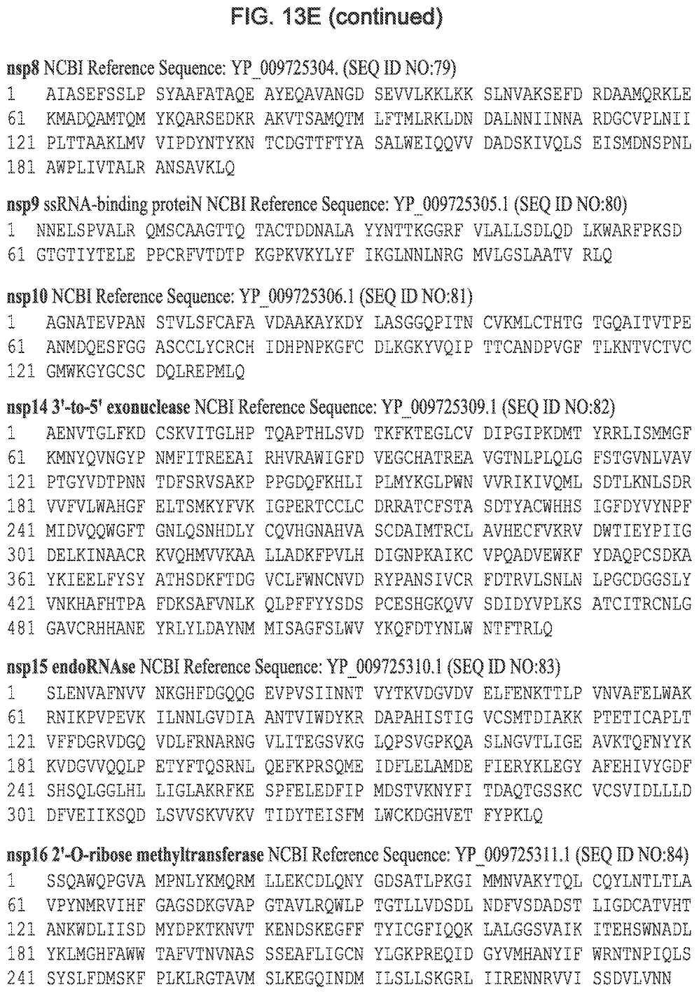

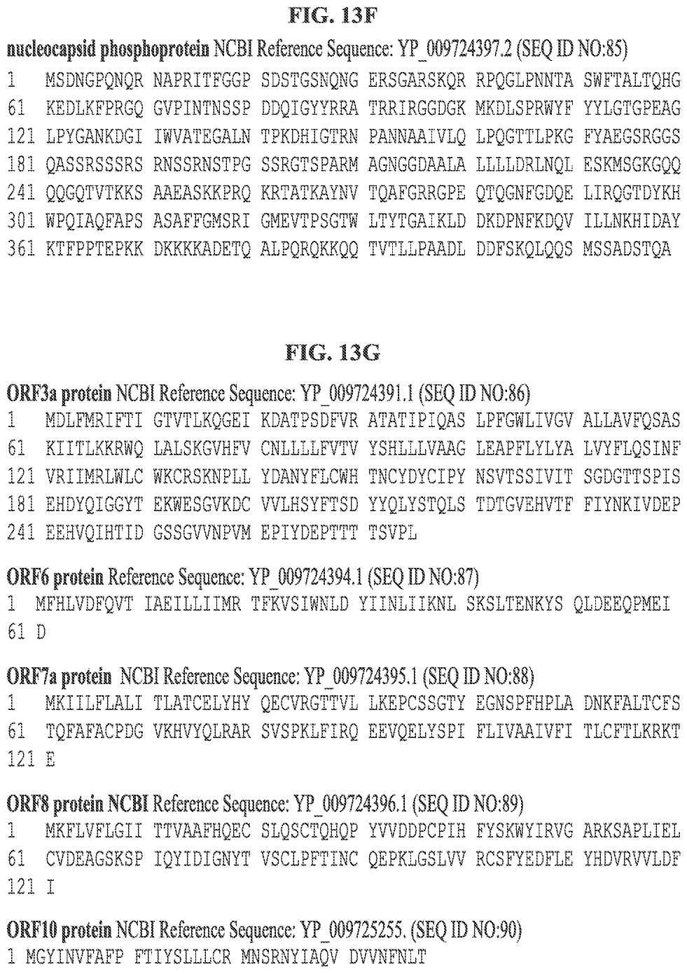

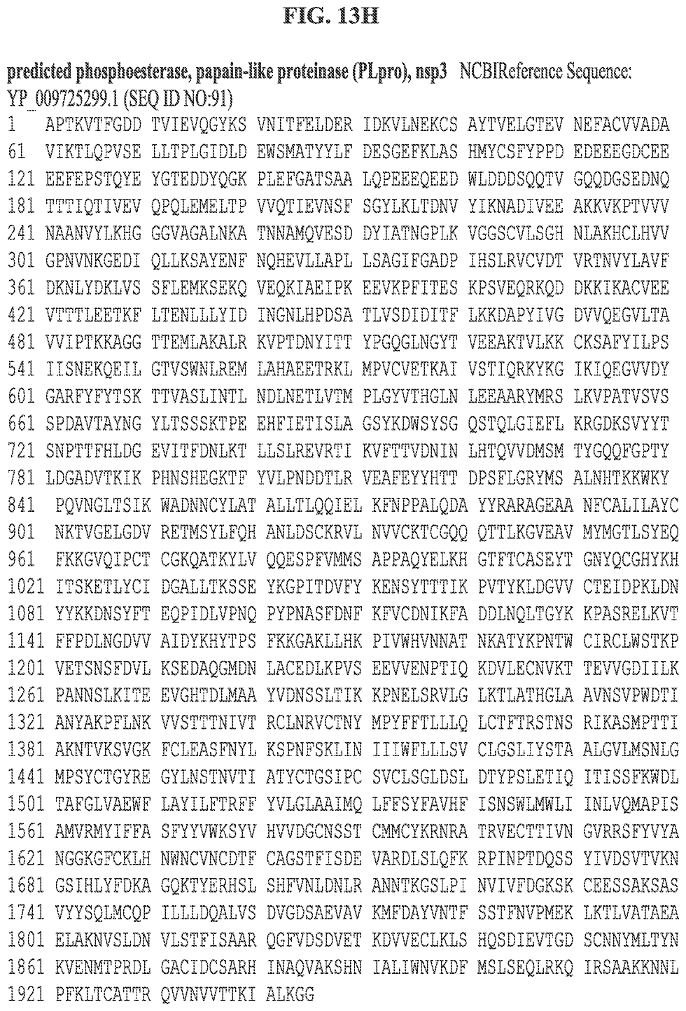

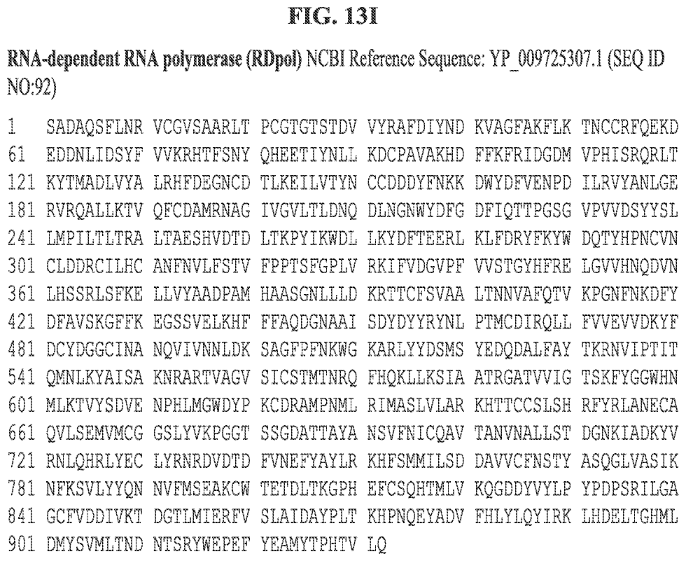

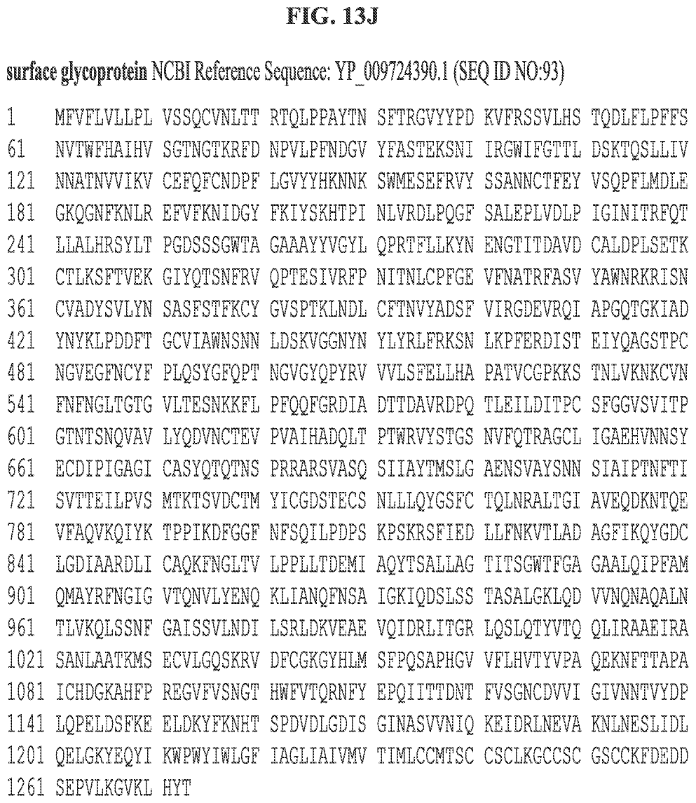

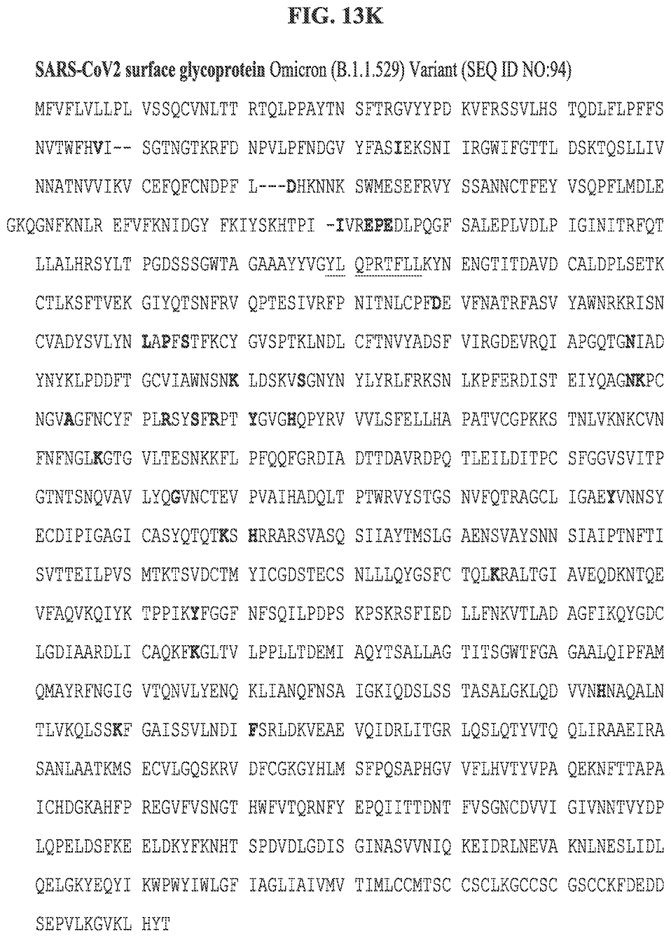

In some cases, a peptide present in a TMMP is a peptide of from 4 amino acids (aa) to 25 aa in length (e.g., 4 aa, 5 aa, 6 aa, 7, aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa in length) of a polypeptide comprising an amino acid sequence having at least 50%, at least 60%, at least 70%, at least 75%, at least 80%, at least 85%, at least 90%, at least 95%, at least 98%, at least 99%, or 100%, amino acid sequence identity to any one of the SARS-CoV-2 polypeptides depicted in FIG. 13A-13J. In some cases, a peptide present in a TMMP is a peptide of from 4 amino acids (aa) to 25 aa in length (e.g., 4 aa, 5 aa, 6 aa, 7, aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa in length) of one of the polypeptides depicted in FIG. 13A-13J.

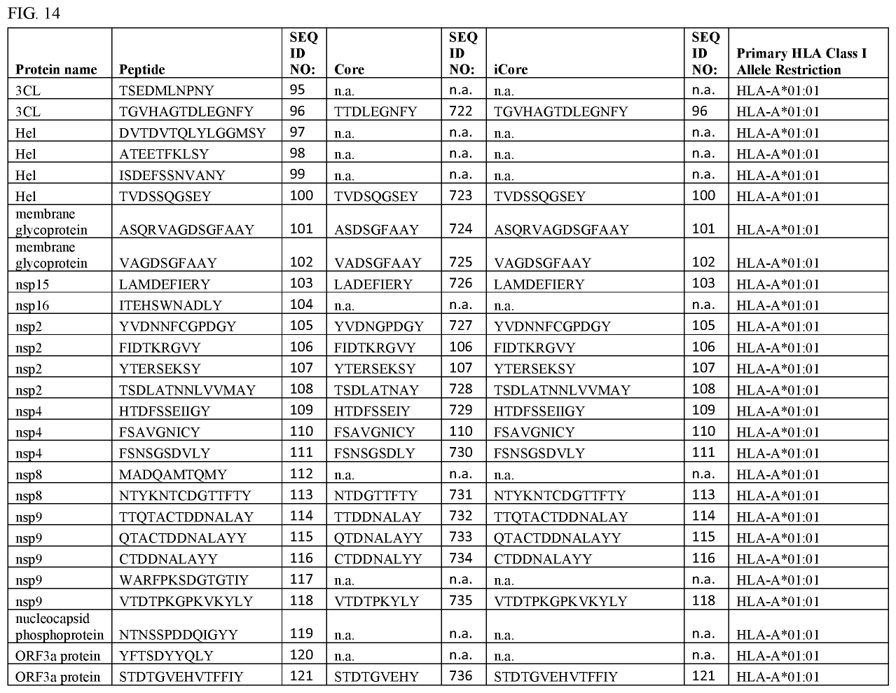

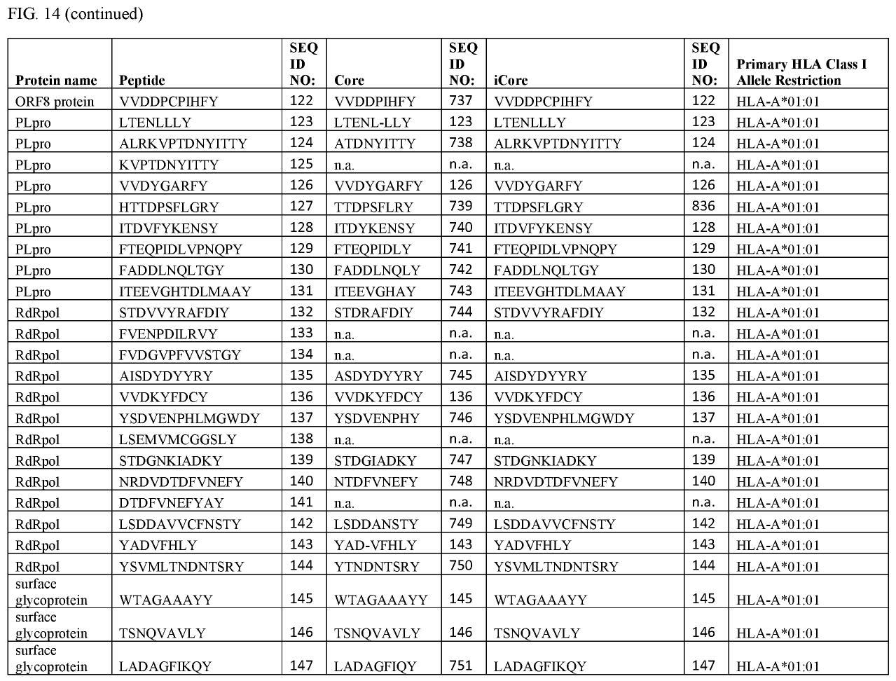

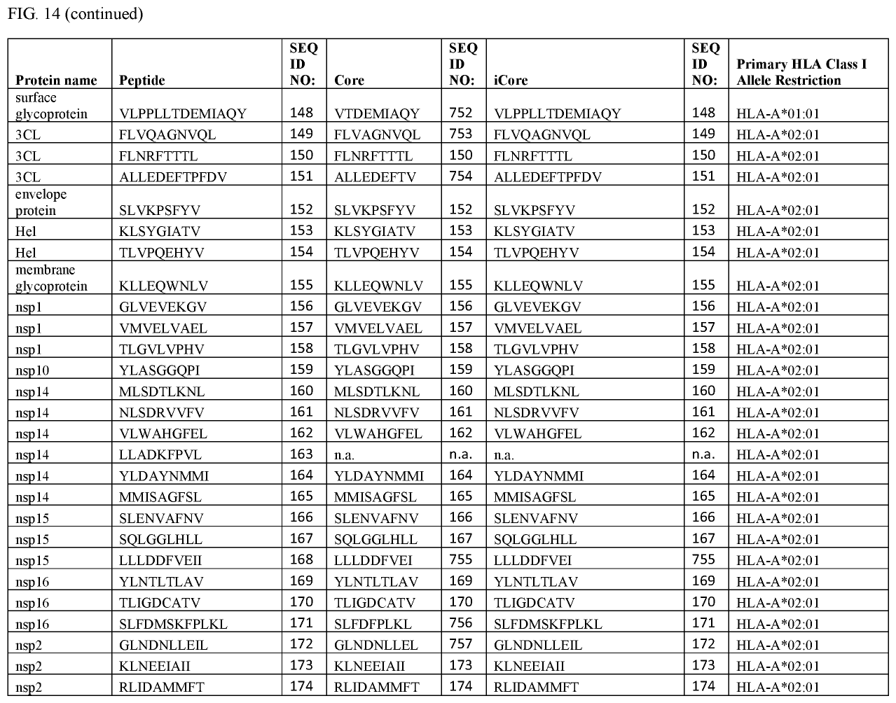

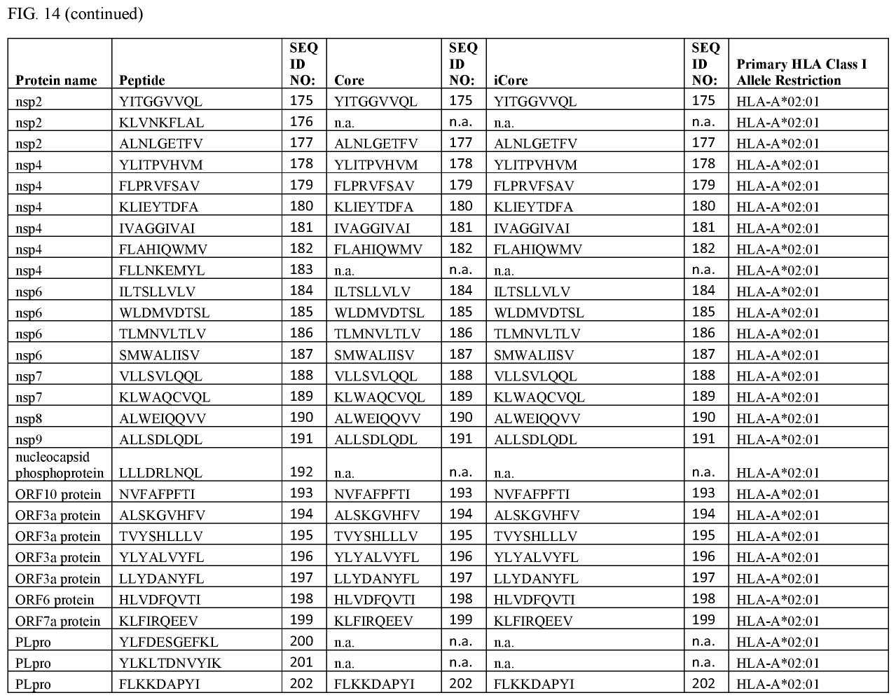

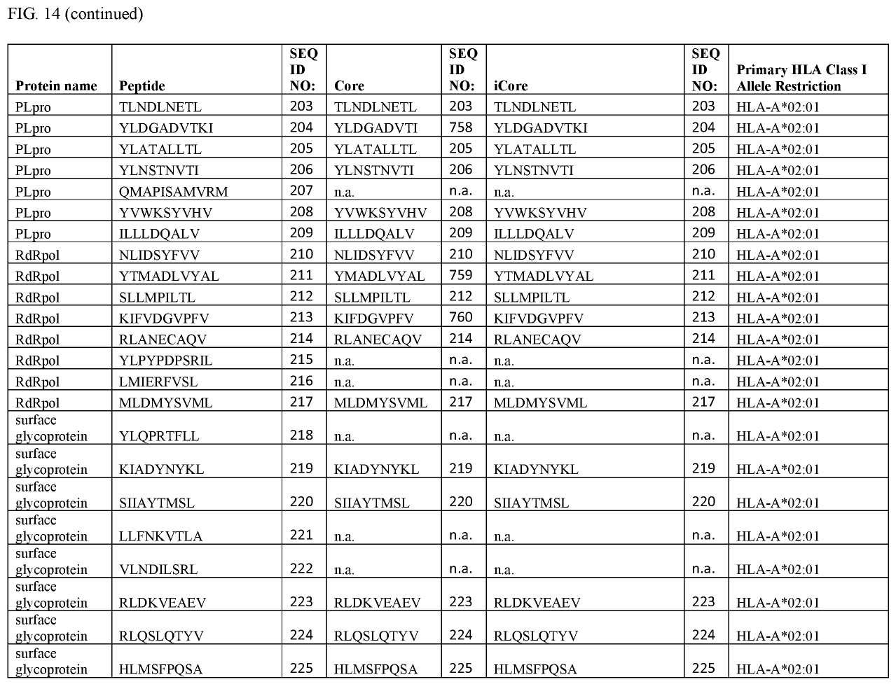

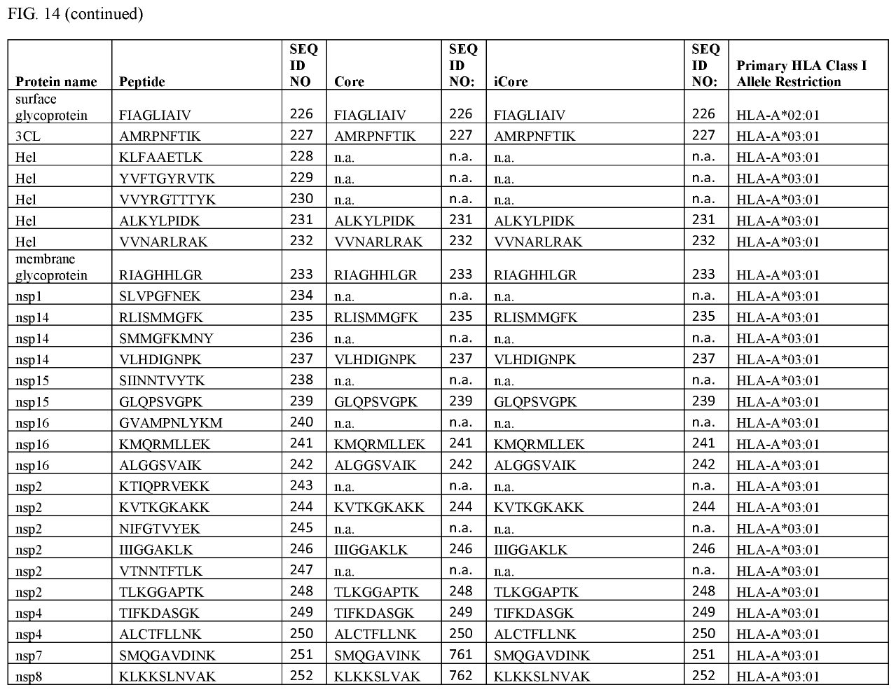

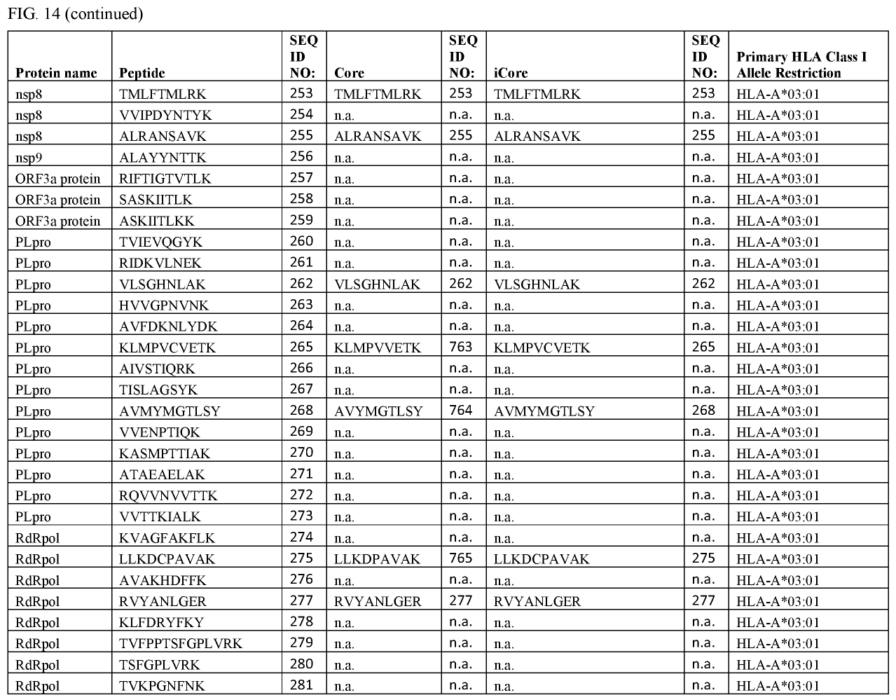

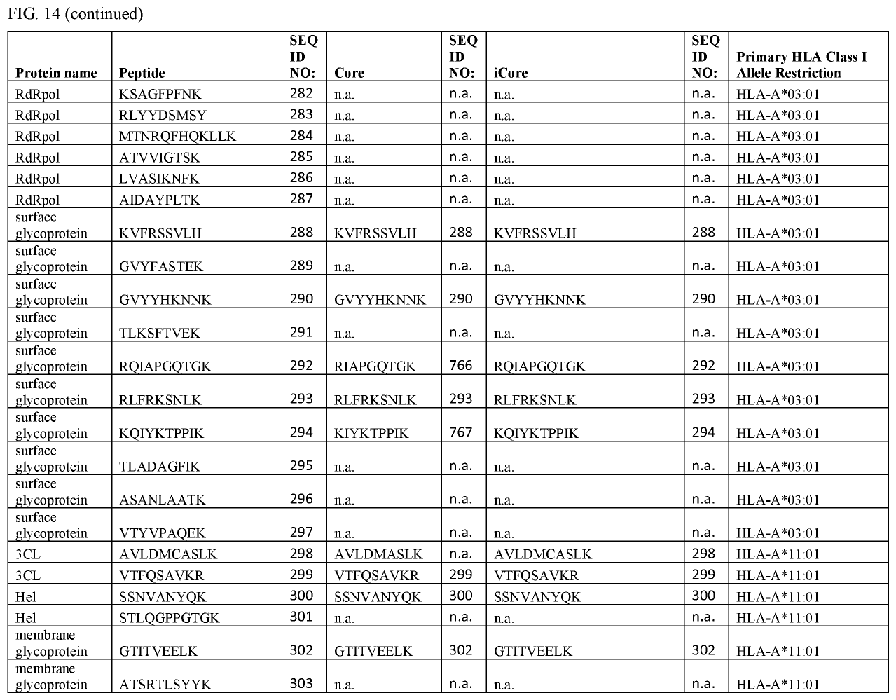

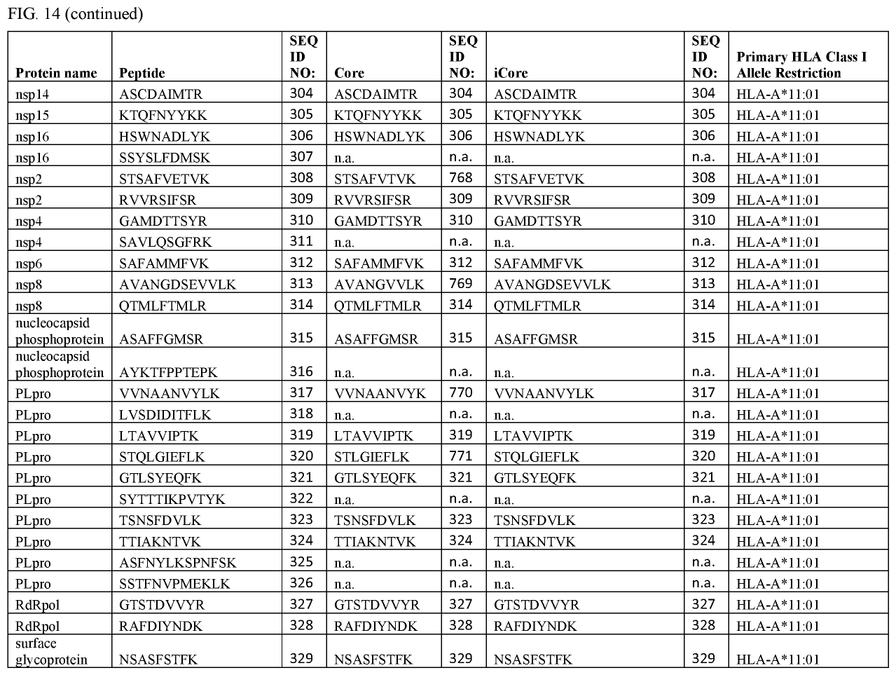

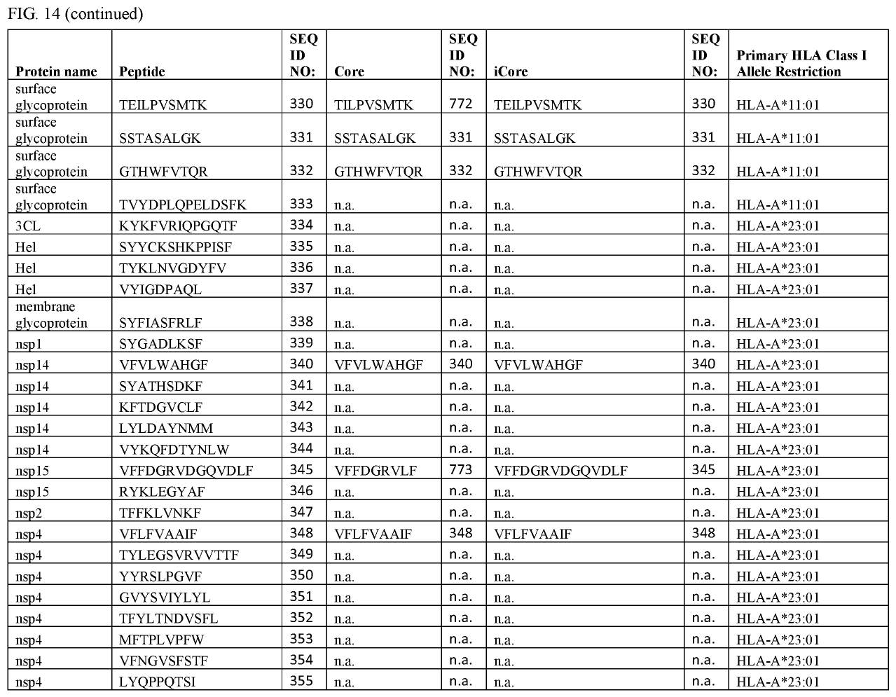

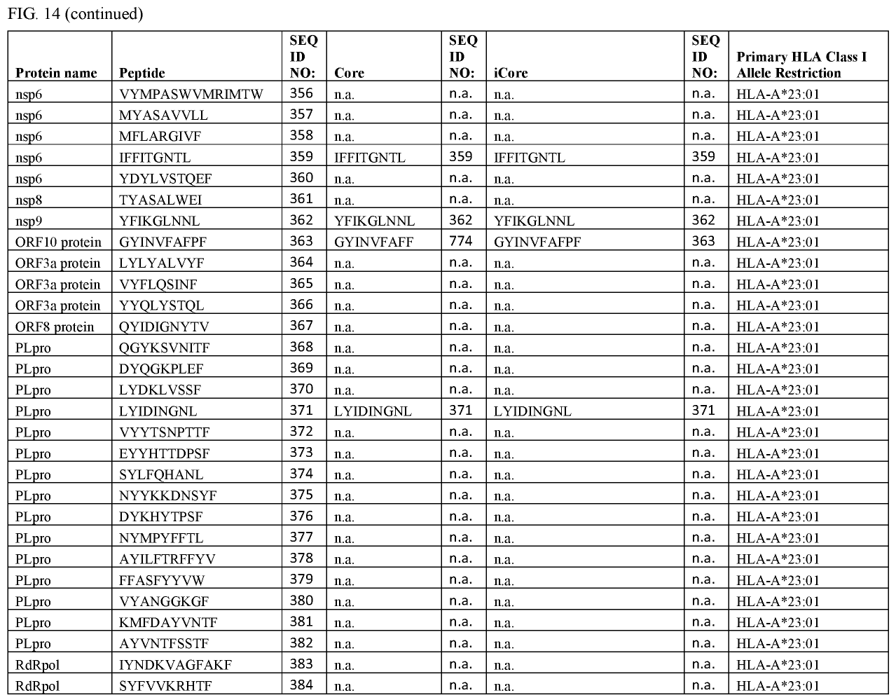

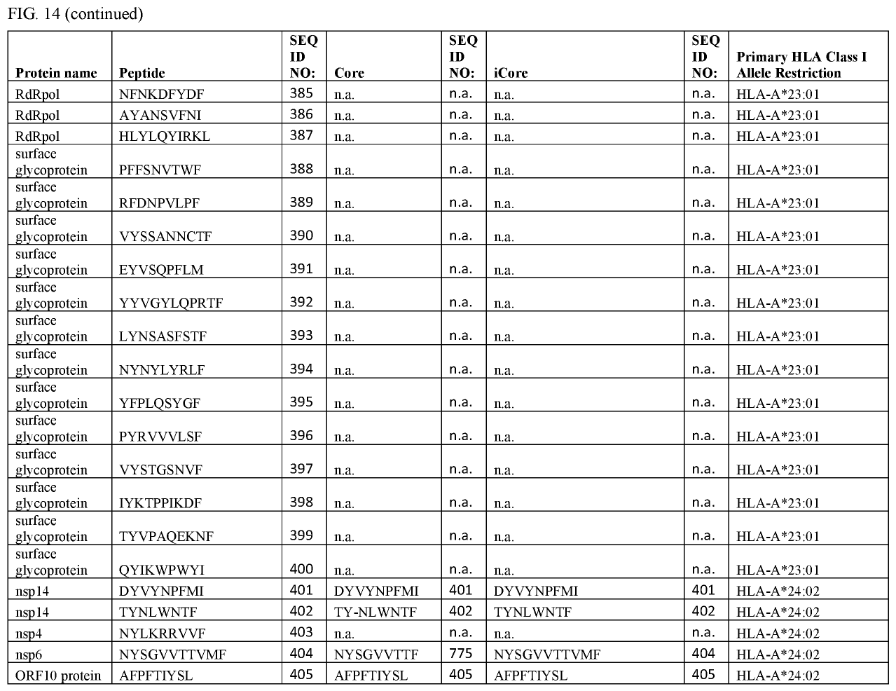

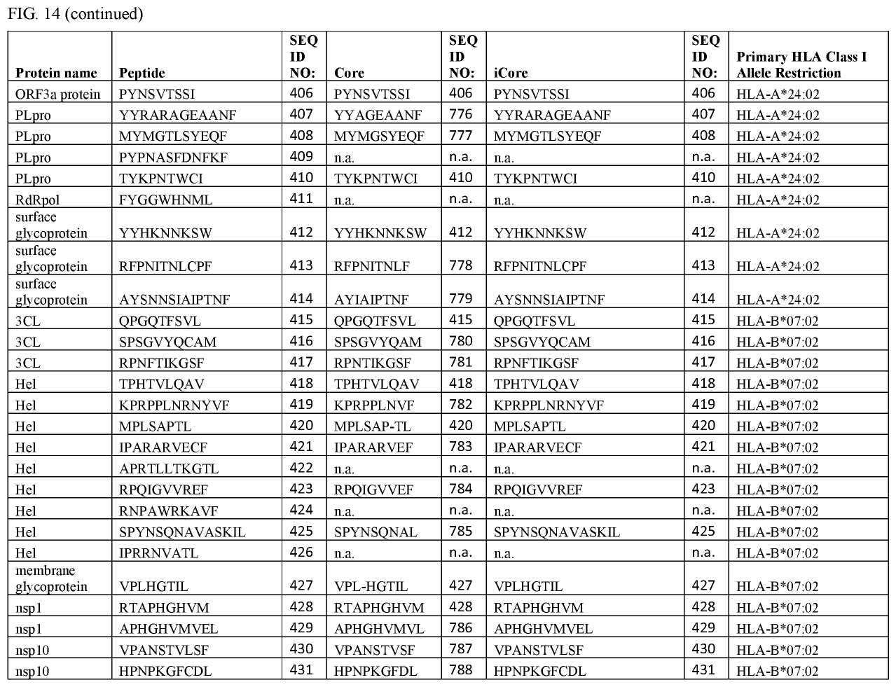

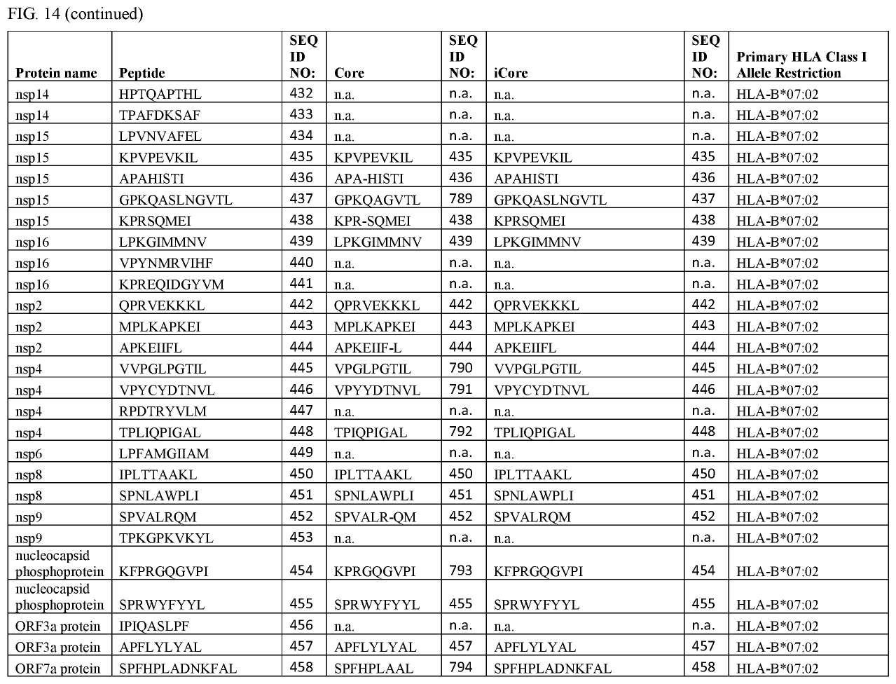

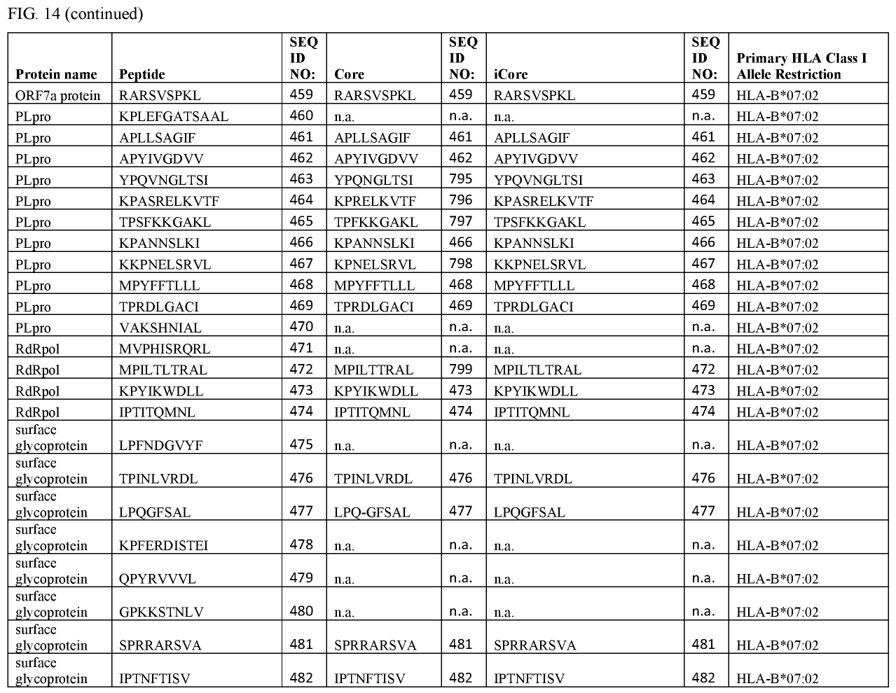

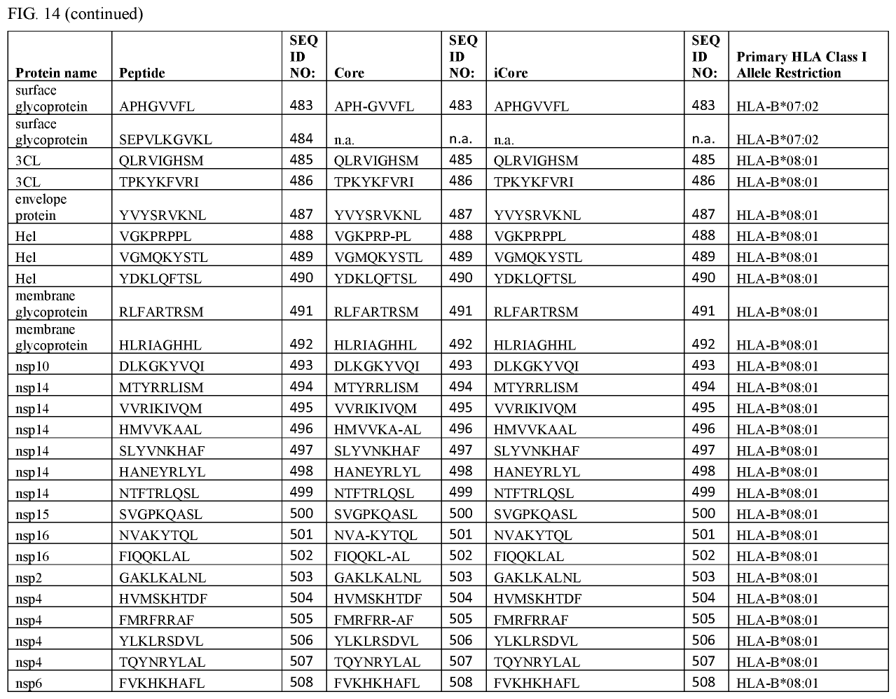

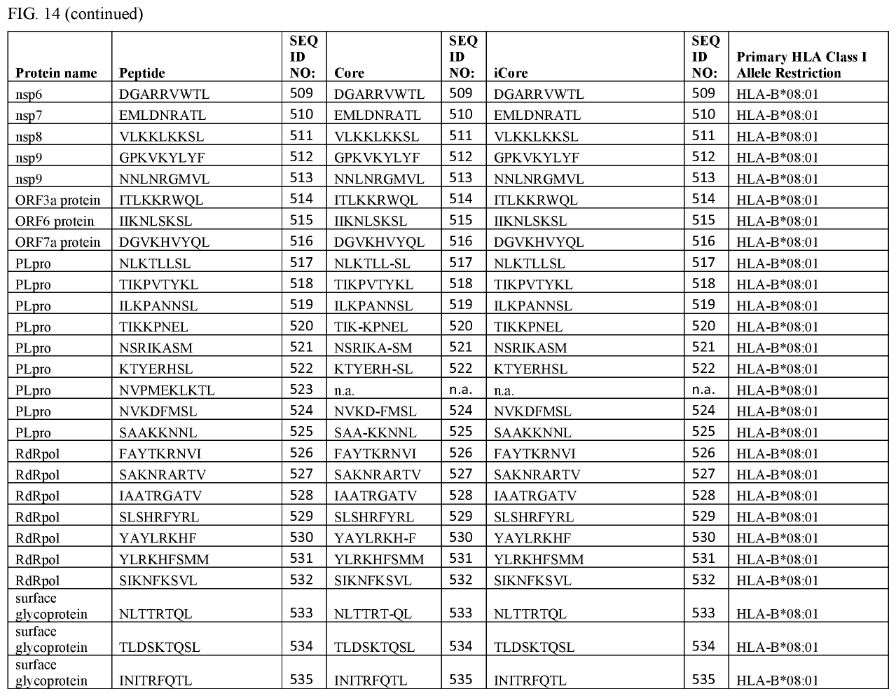

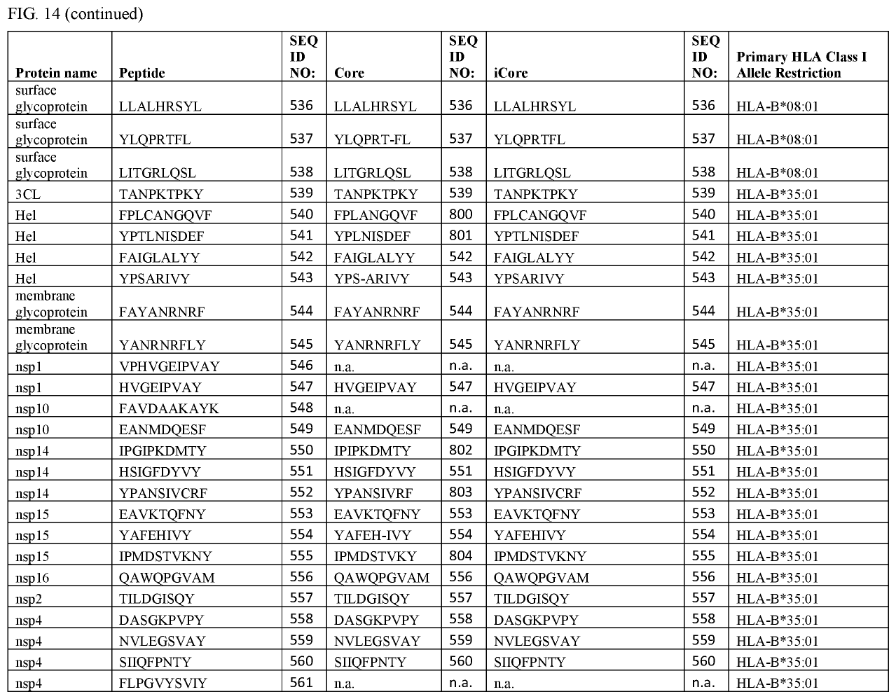

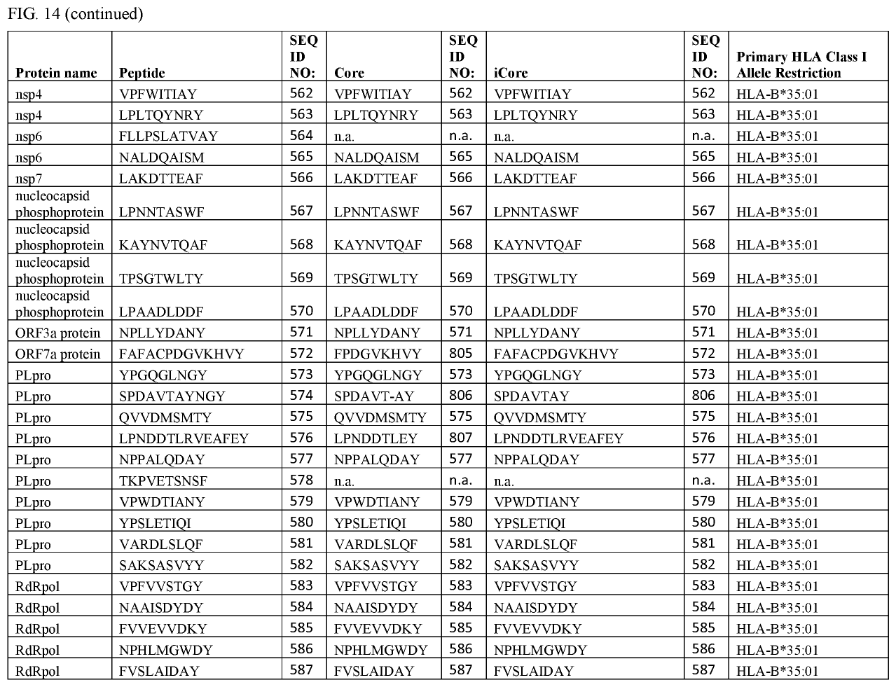

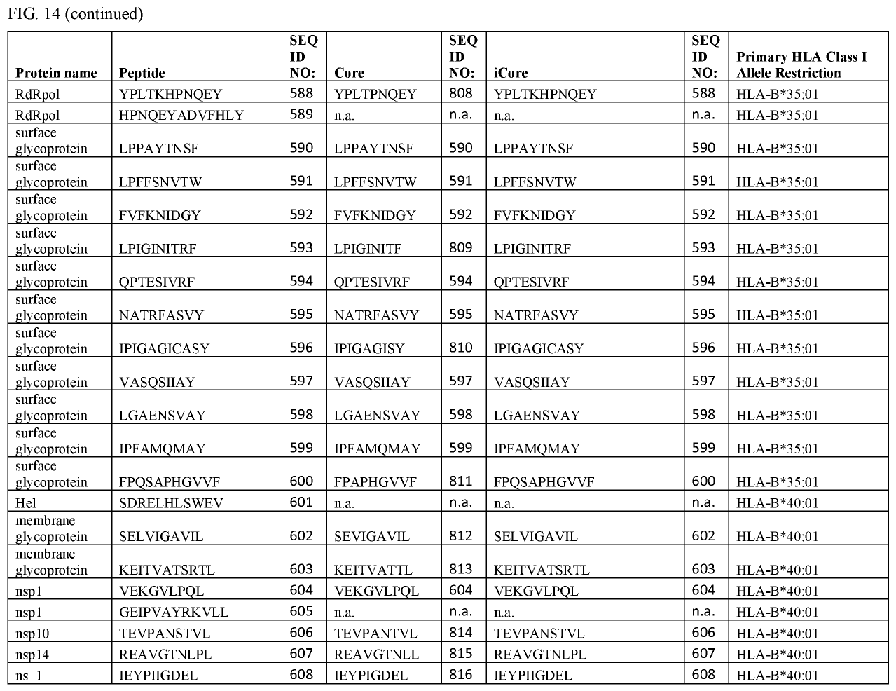

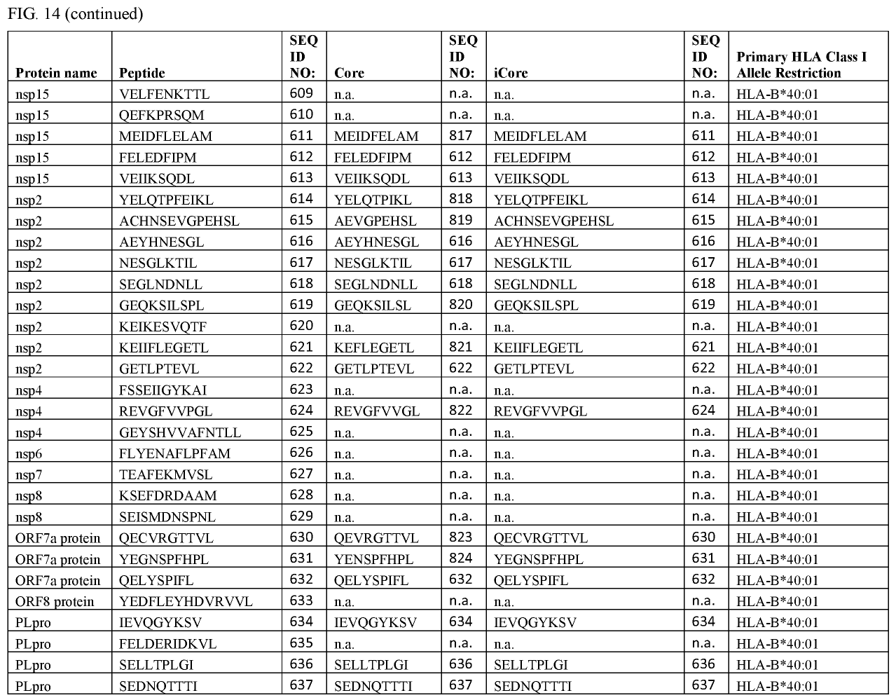

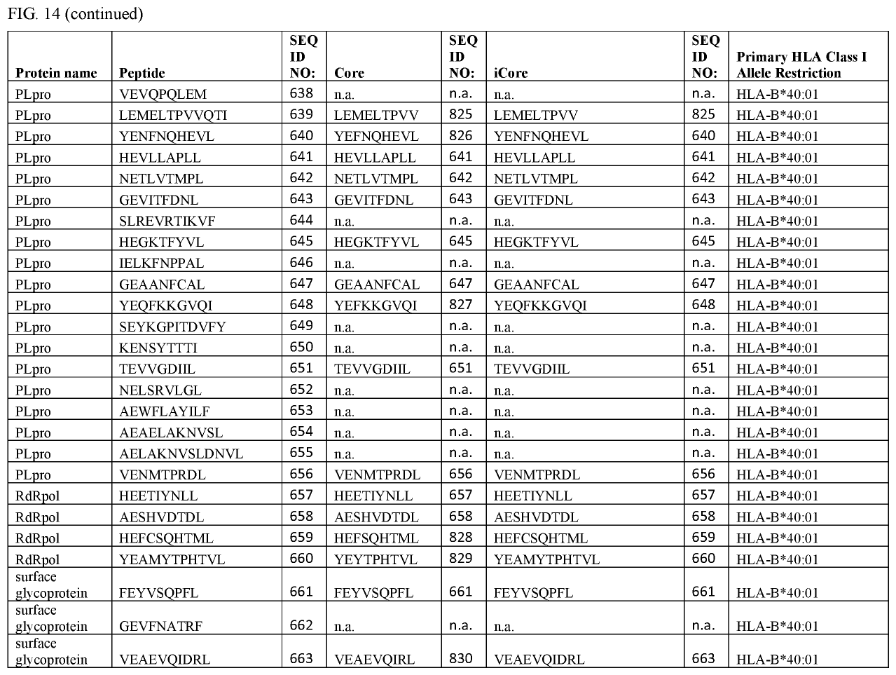

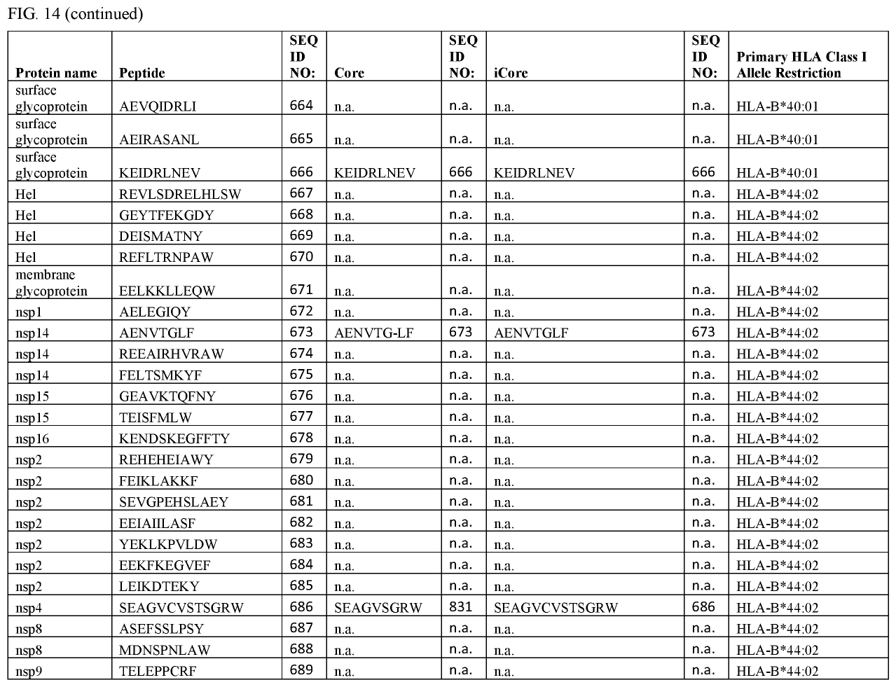

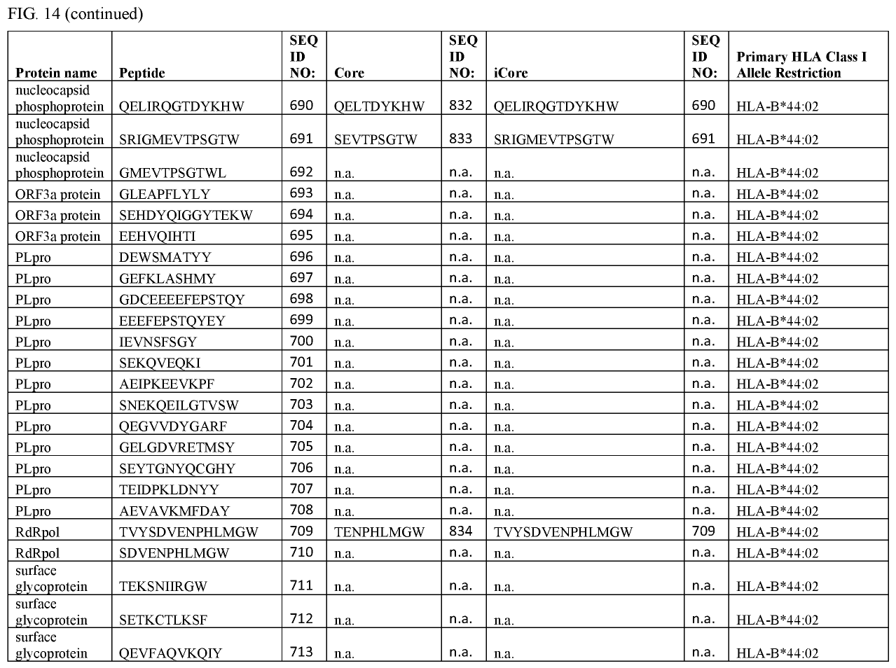

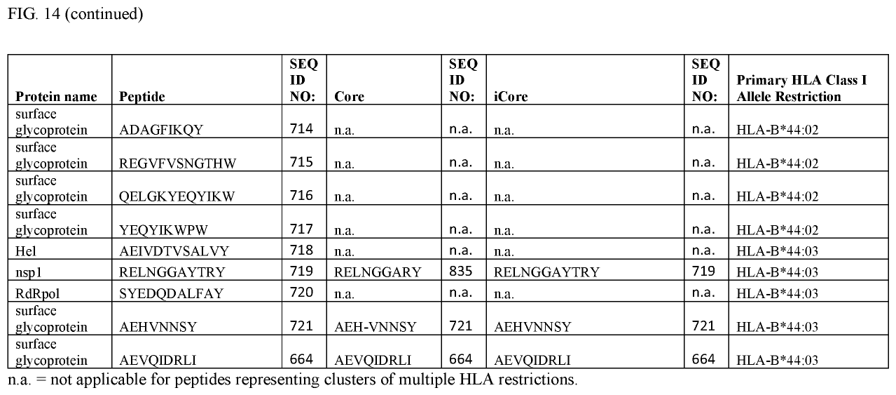

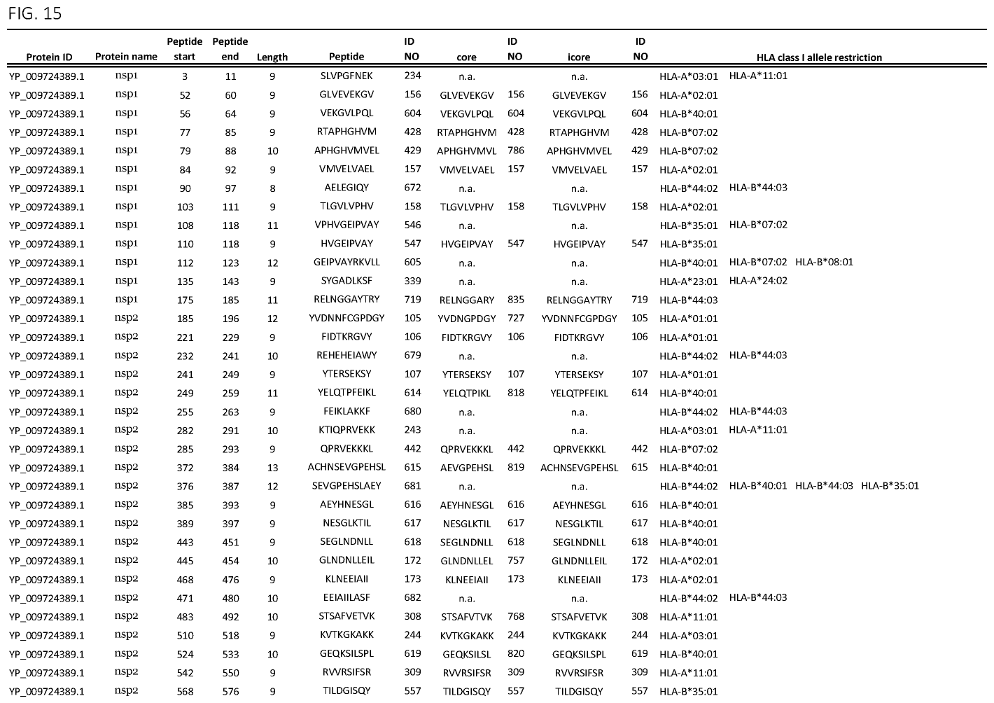

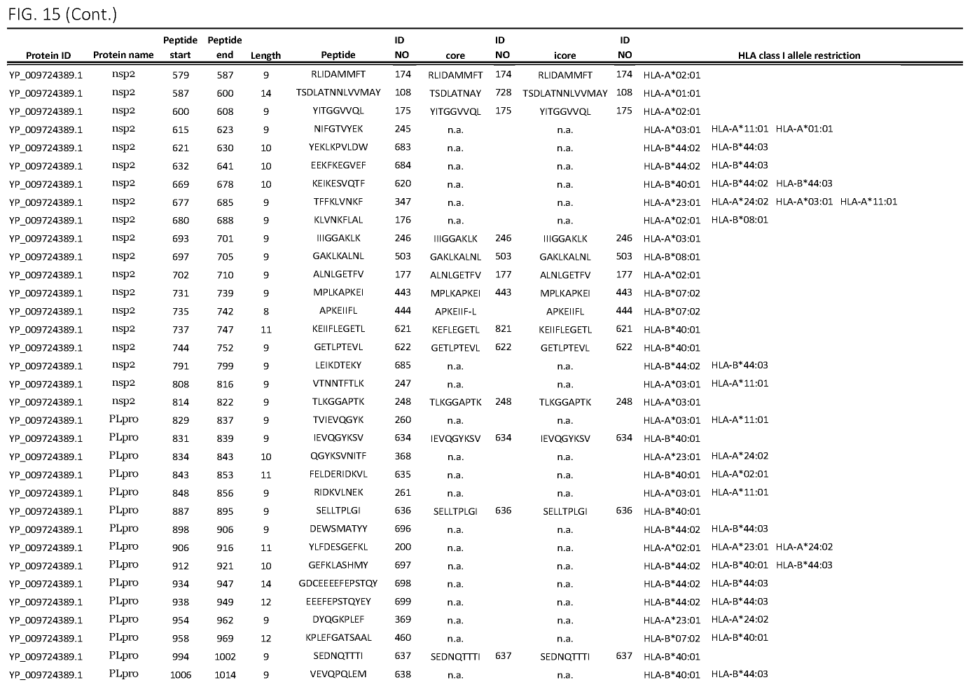

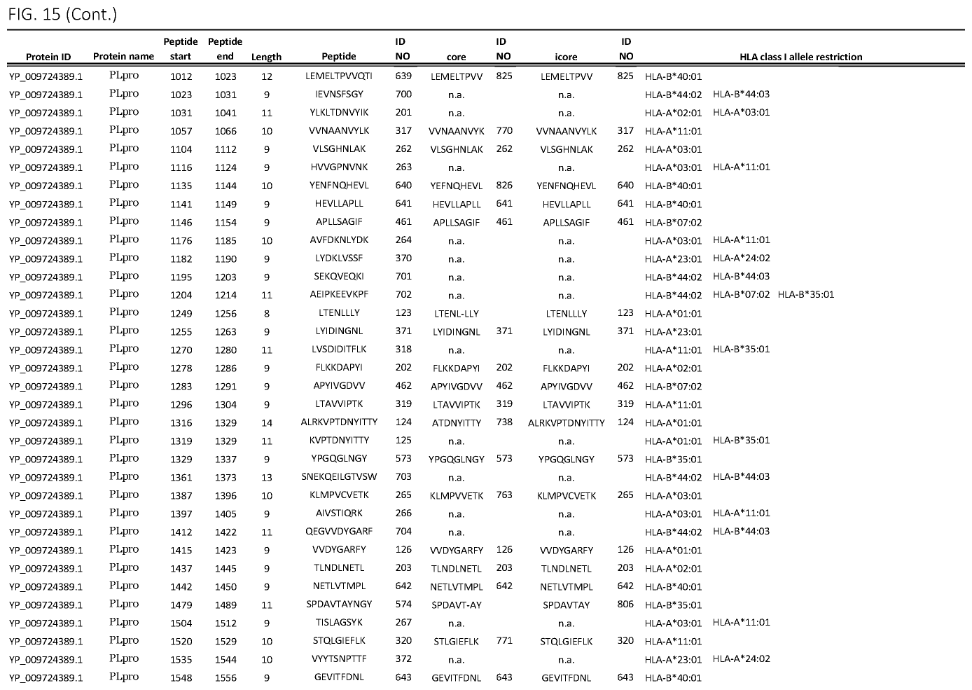

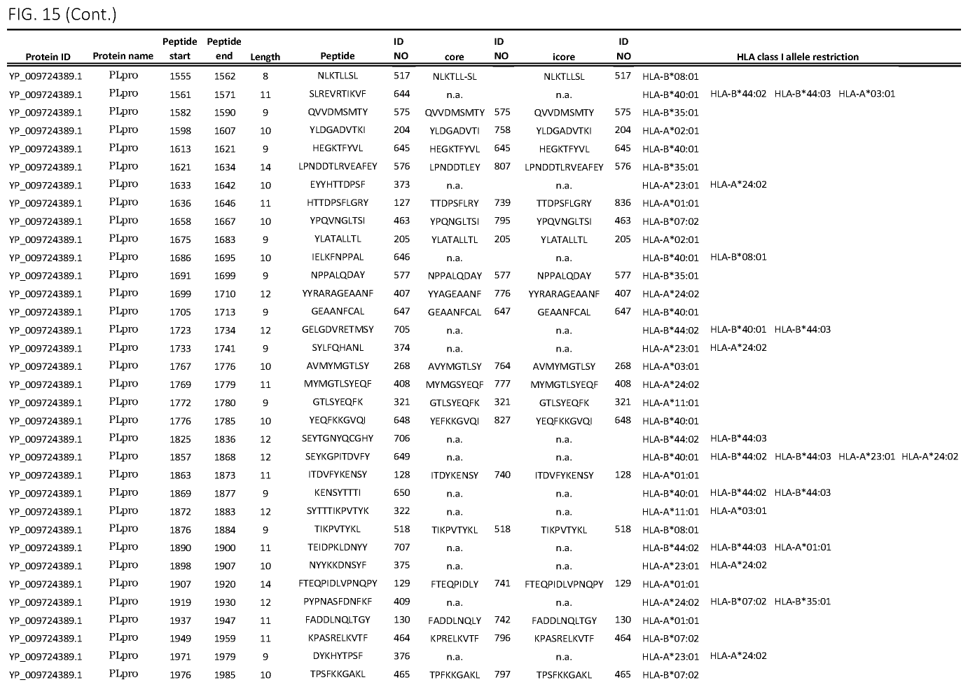

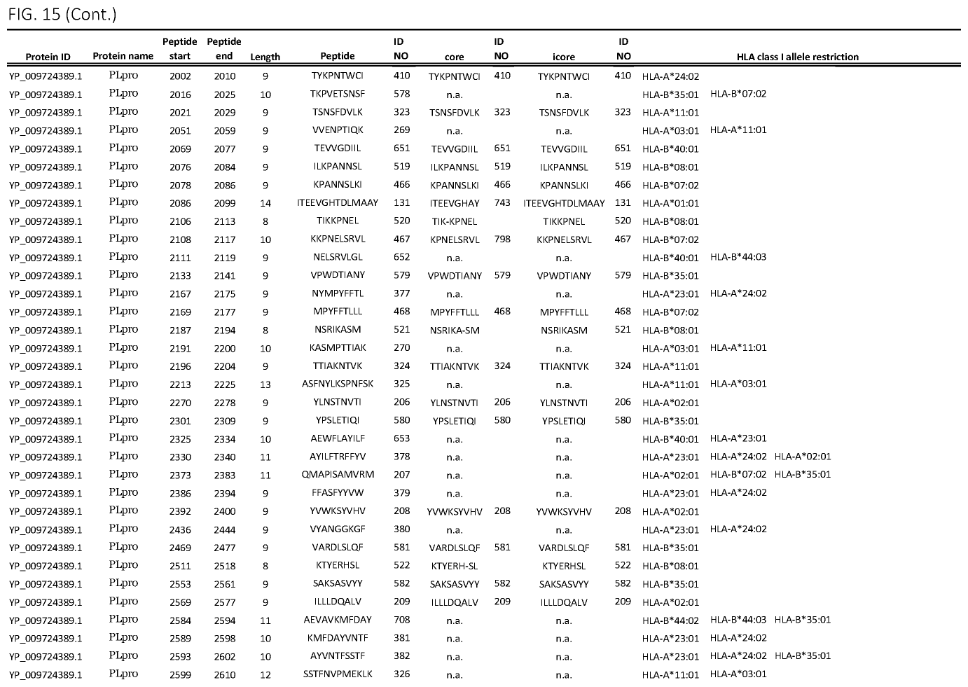

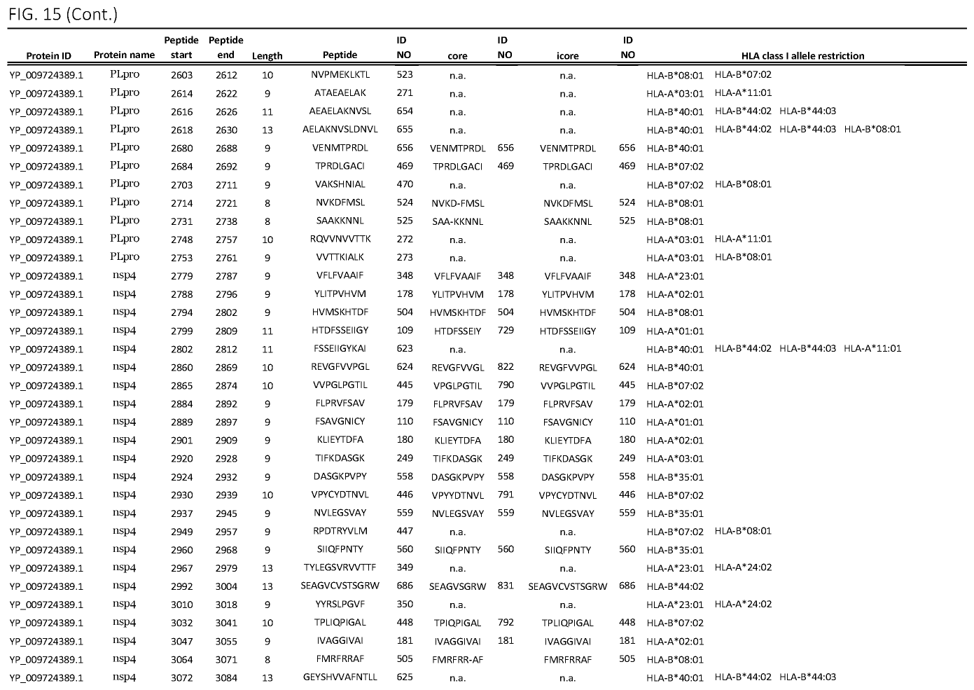

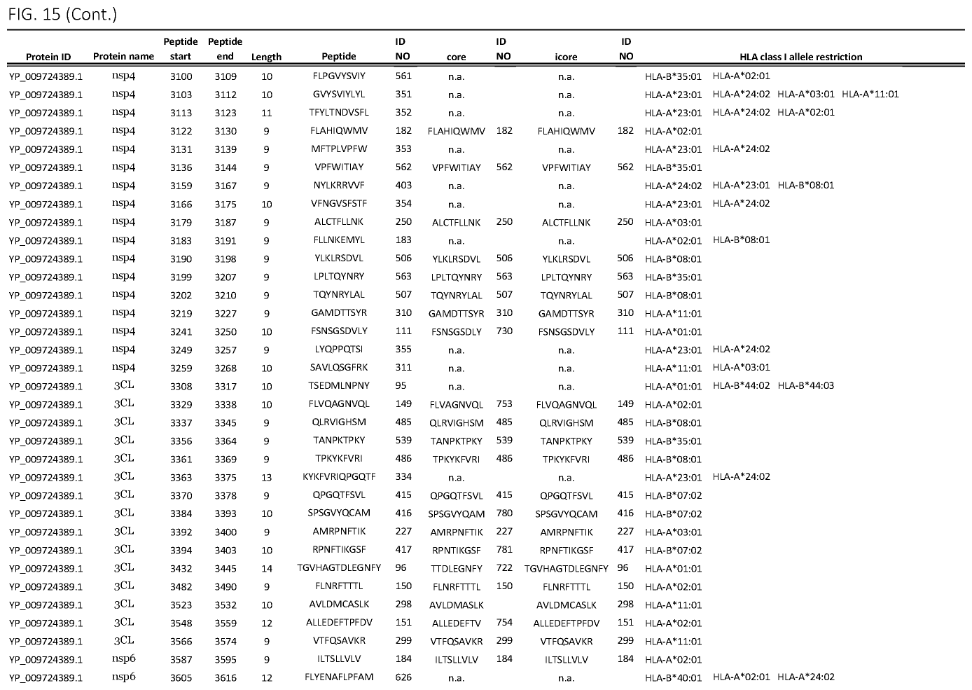

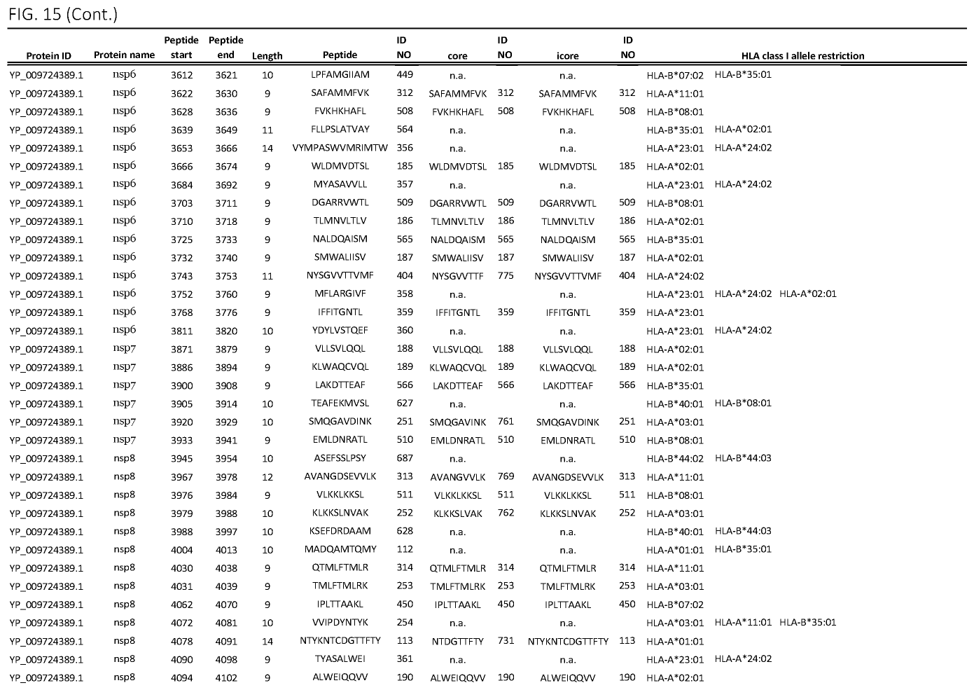

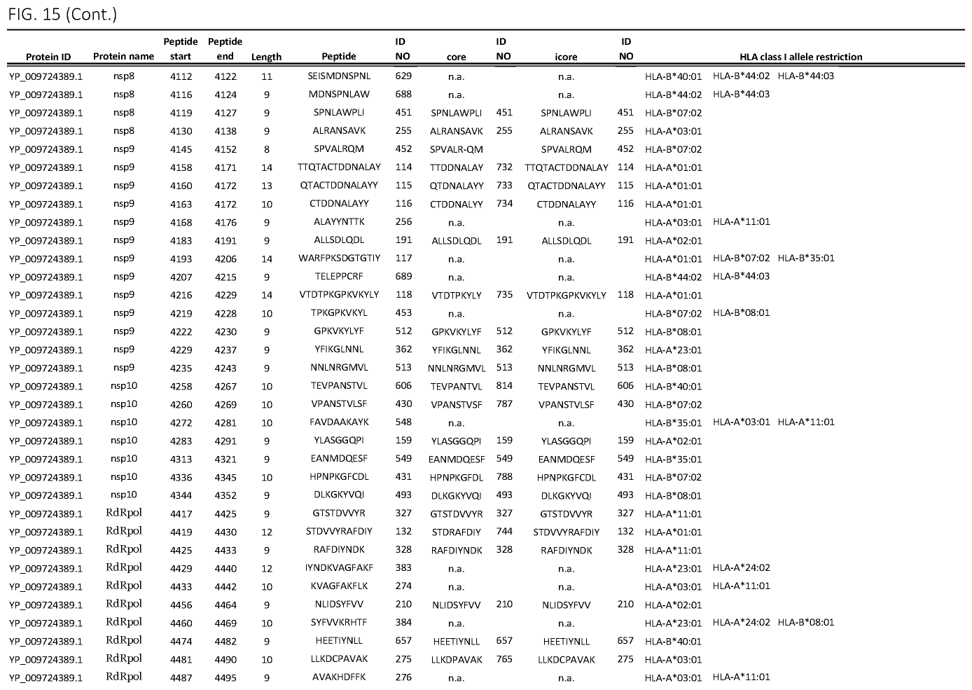

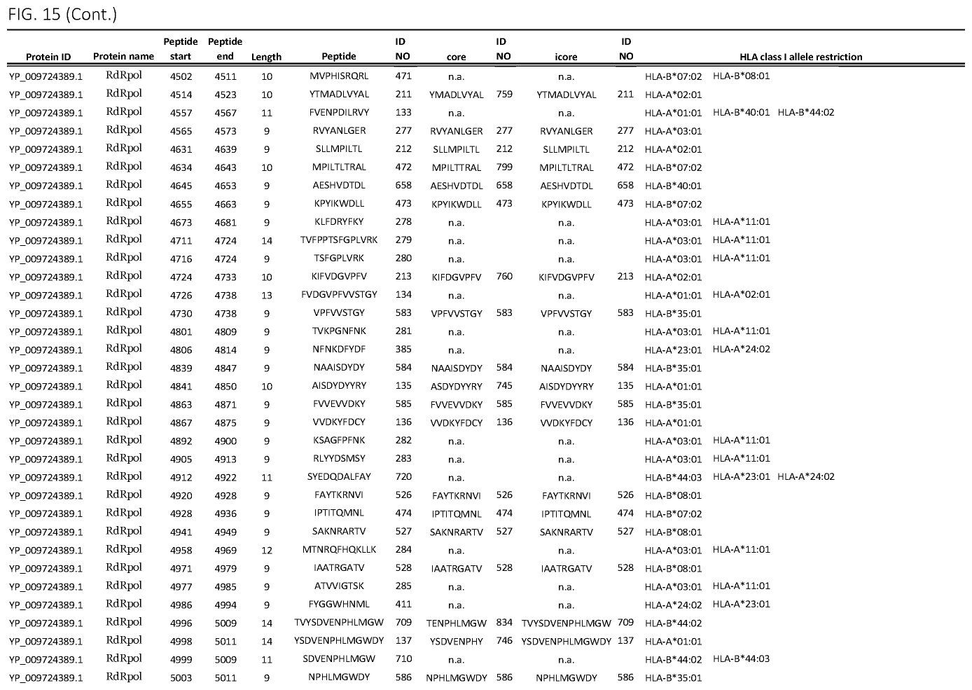

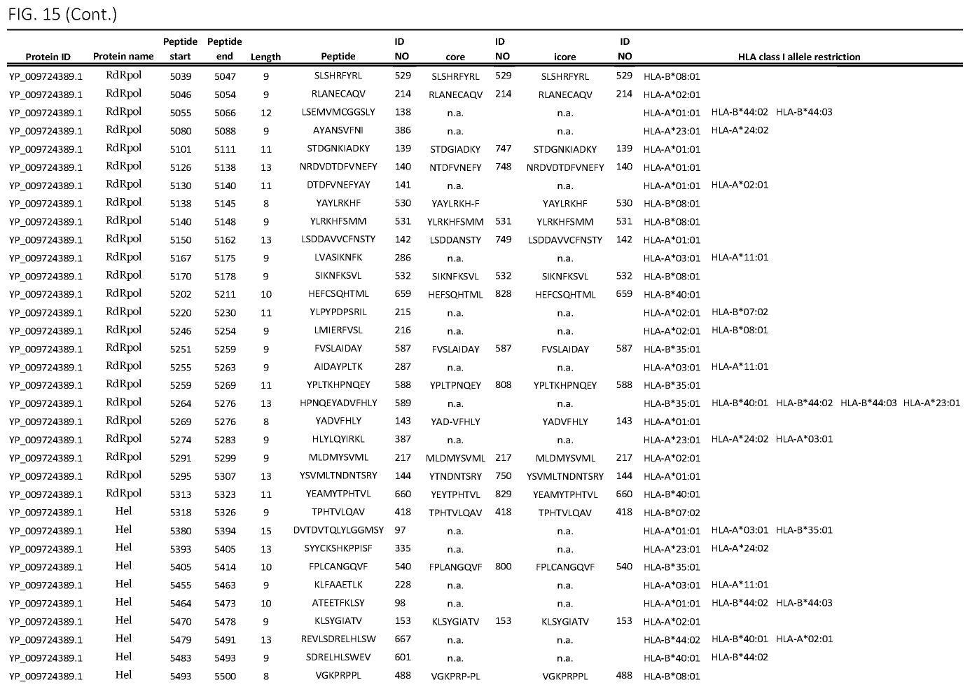

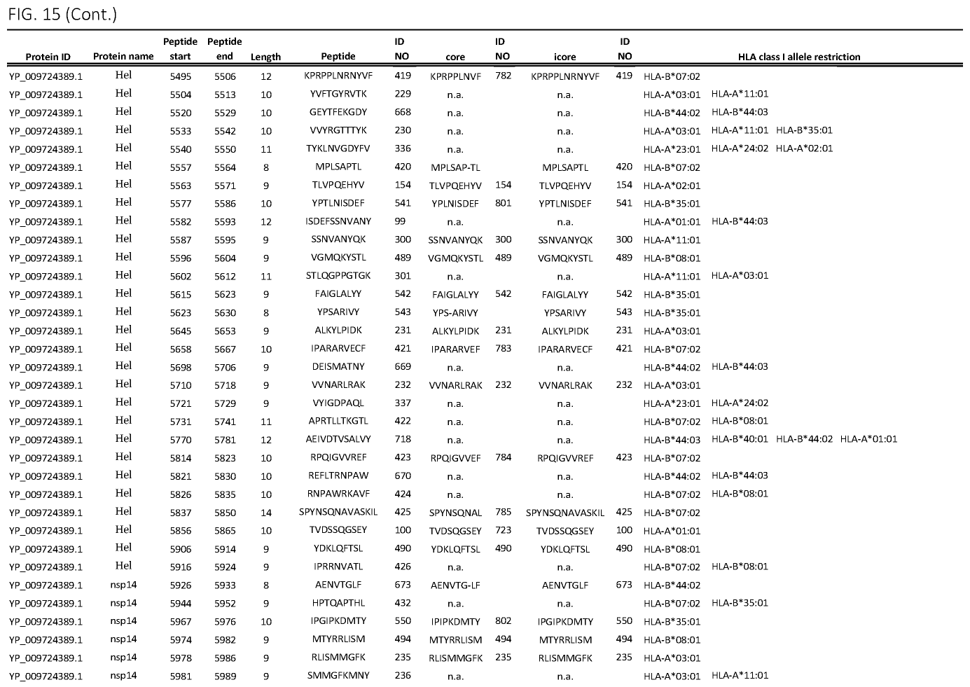

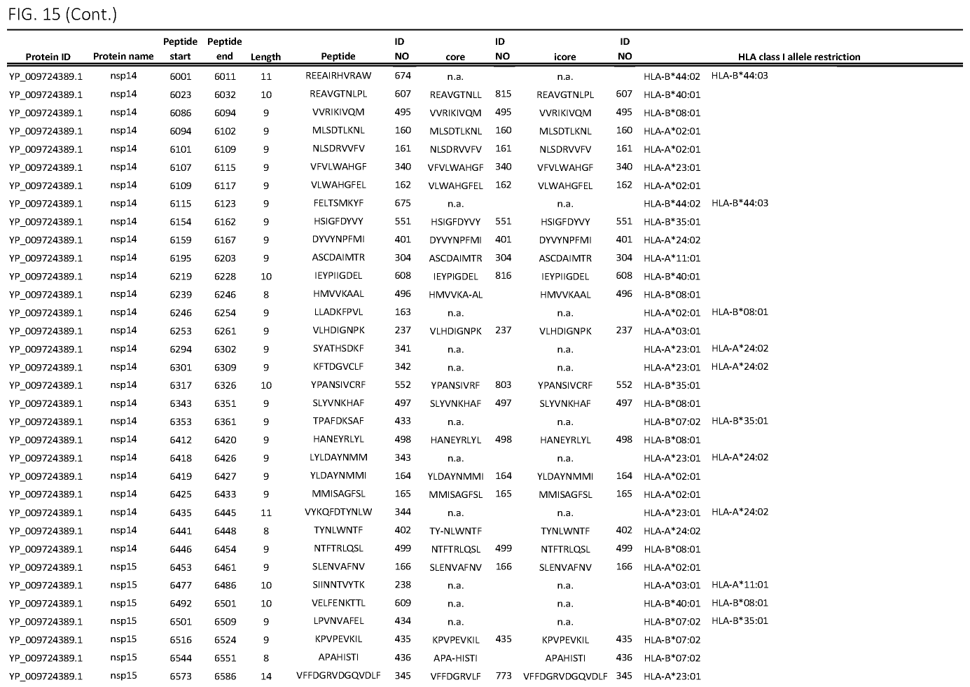

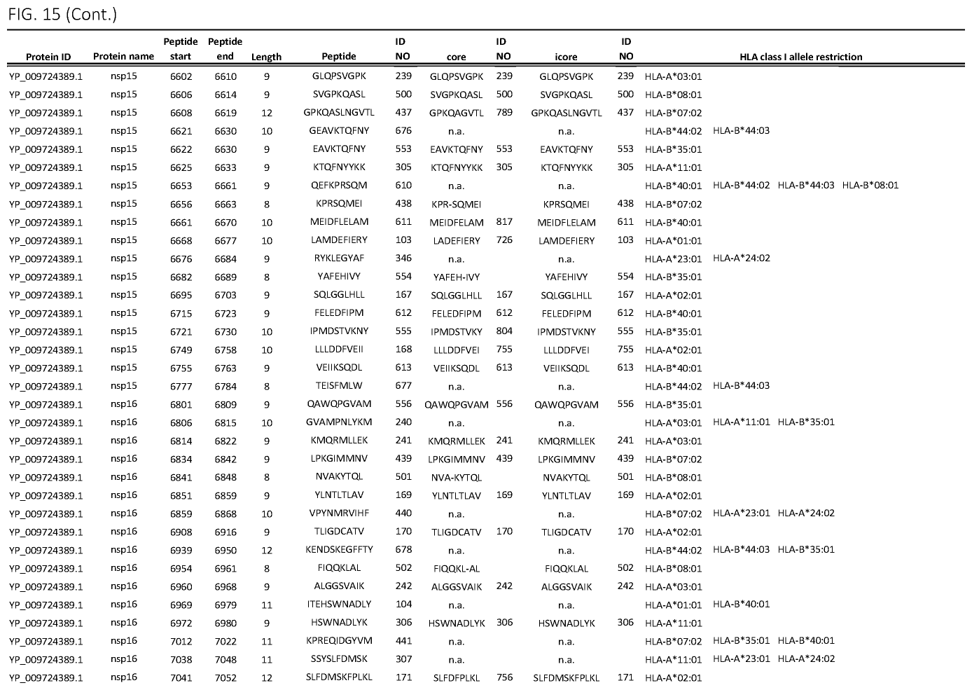

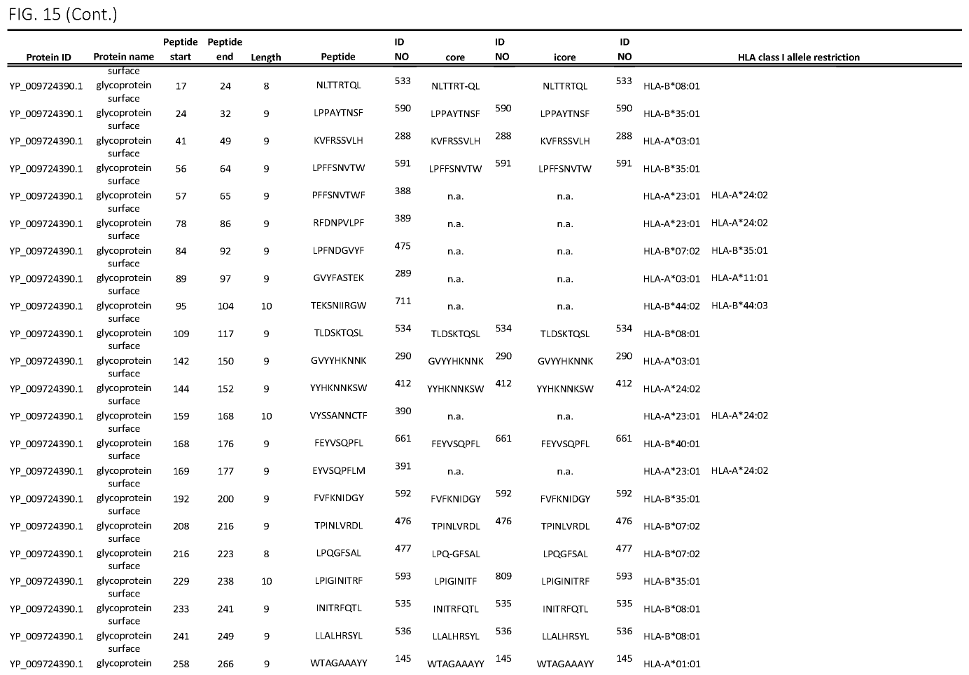

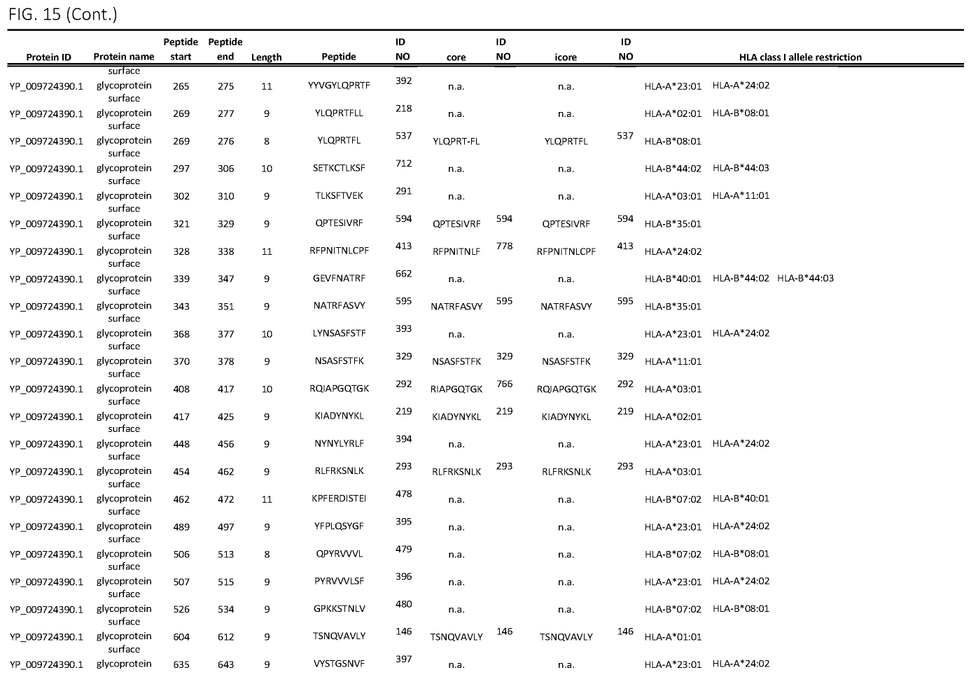

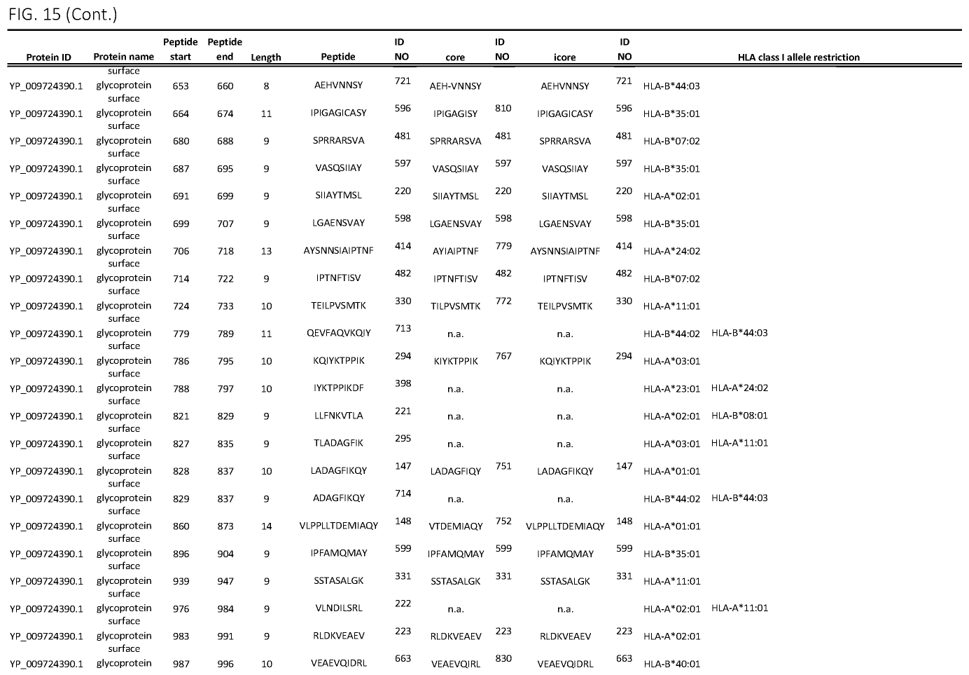

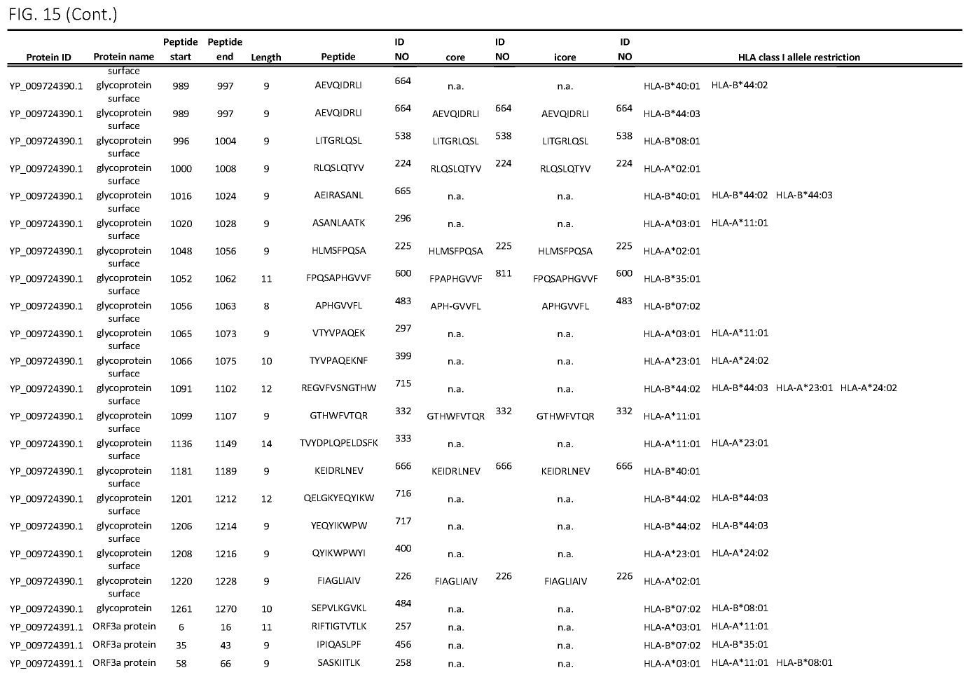

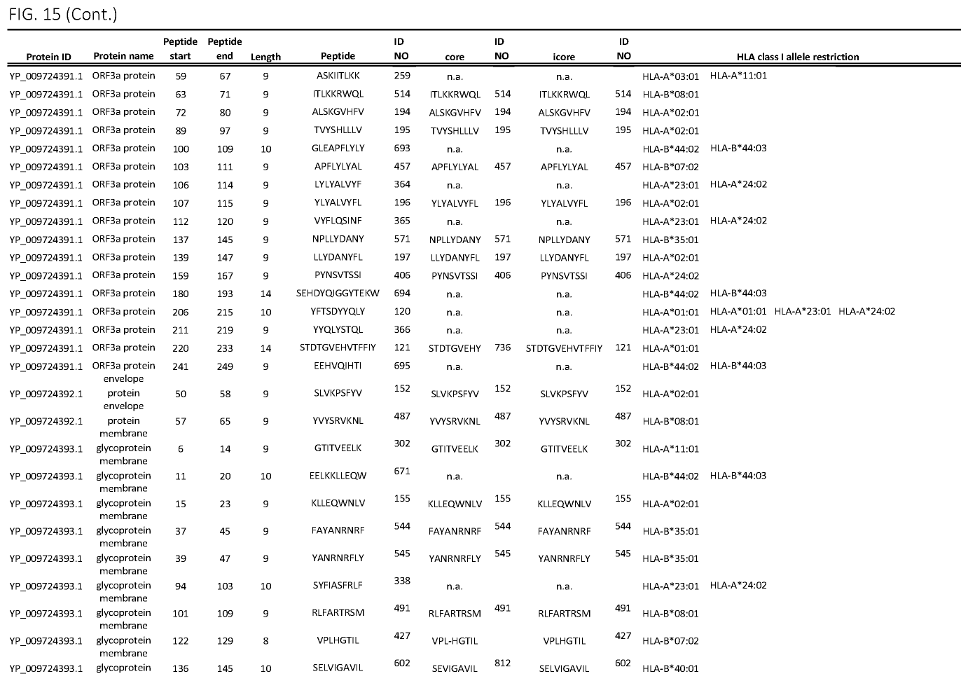

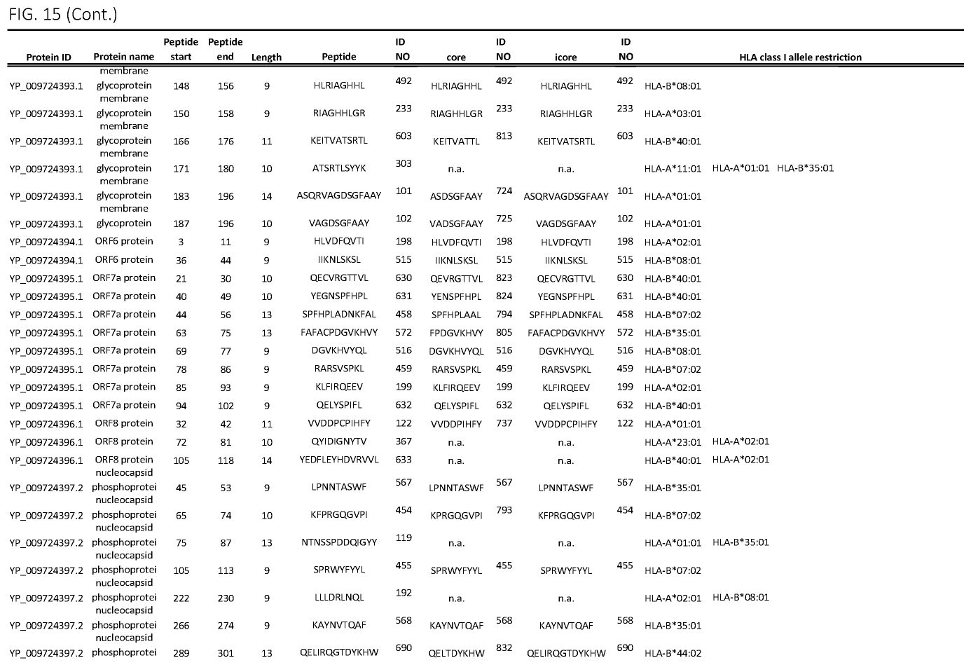

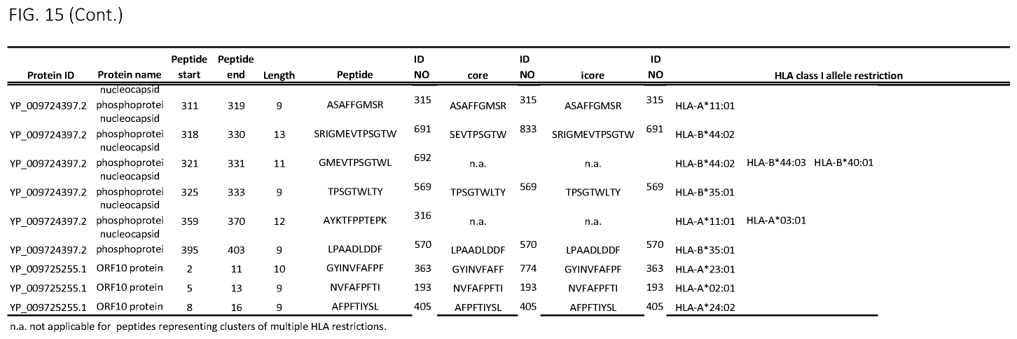

In some cases, a peptide present in a TMMP is any one of the peptides depicted in FIG. 14. In some cases, a peptide present in a TMMP is any one of the peptides depicted in FIG. 15.

In some cases, the peptide epitope is a SARS-CoV-2 peptide from a SARS-CoV-2-encoded surface glycoprotein. In some cases, a peptide present in a TMMP is a peptide of from 4 amino acids (aa) to 25 aa in length (e.g., 4 aa, 5 aa, 6 aa, 7, aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa in length) of a polypeptide comprising an amino acid sequence having at least 50%, at least 60%, at least 70%, at least 75%, at least 80%, at least 85%, at least 90%, at least 95%, at least 98%, at least 99%, or 100%, amino acid sequence identity to the SARS-CoV-2 surface glycoprotein depicted in FIG. 13J. In some cases, a peptide present in a TMMP is a peptide of from 4 amino acids (aa) to 25 aa in length (e.g., 4 aa, 5 aa, 6 aa, 7, aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa in length) of the SARS-CoV-2 surface glycoprotein depicted in FIG. 13J.

In some cases, the peptide epitope is a SARS-CoV-2 peptide from a SARS-CoV-2-encoded surface glycoprotein, where the SARS-CoV-2 is the omicron variant (also known as the B1.1.529 variant). In some cases, a peptide present in a TMMP is a peptide of from 4 amino acids (aa) to 25 aa in length (e.g., 4 aa, 5 aa, 6 aa, 7, aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa in length) of a polypeptide comprising an amino acid sequence having at least 50%, at least 60%, at least 70%, at least 75%, at least 80%, at least 85%, at least 90%, at least 95%, at least 98%, at least 99%, or 100%, amino acid sequence identity to the SARS-CoV-2 surface glycoprotein depicted in FIG. 13K. As shown in FIG. 13K, the spike glycoprotein of the omicron variant includes the following changes: A67V, de169-70, T951, de1142-144, Y145D, del211, L212I, ins214EPE, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, L981F, where the substitutions in bold are in the receptor binding domain (RBD), where “ins” refers to an insertion, and where “del” refers to a deletion. In some cases, a peptide present in a TMMP is a peptide of from 4 amino acids (aa) to 25 aa in length (e.g., 4 aa, 5 aa, 6 aa, 7, aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa in length) of the SARS-CoV-2 surface glycoprotein depicted in FIG. 13K.

In some cases, a peptide present in a TMMP is a SARS-CoV-2 peptide from a SARS-CoV-2-encoded surface glycoprotein and is selected from the group consisting of: NLTTRTQL (SEQ ID NO:533), LPPAYTNSF (SEQ ID NO:590), KVFRSSVLH (SEQ ID NO:288), LPFFSNVTW (SEQ ID NO:591), PFFSNVTWF (SEQ ID NO:388), RFDNPVLPF (SEQ ID NO:389), LPFNDGVYF (SEQ ID NO:475), GVYFASTEK (SEQ ID NO:289), TEKSNIIRGW (SEQ ID NO:711), TLDSKTQSL (SEQ ID NO:534), GVYYHKNNK (SEQ ID NO:290), YYHKNNKSW (SEQ ID NO:412), VYSSANNCTF (SEQ ID NO:390), FEYVSQPFL (SEQ ID NO:661), EYVSQPFLM (SEQ ID NO:391), FVFKNIDGY (SEQ ID NO:592), TPINLVRDL (SEQ ID NO:476), LPQGFSAL (SEQ ID NO:477), LPIGINITRF (SEQ ID NO:593), INITRFQTL (SEQ ID NO:535), LLALHRSYL (SEQ ID NO:536), WTAGAAAYY (SEQ ID NO:145), YYVGYLQPRTF (SEQ ID NO:392), YLQPRTFLL (SEQ ID NO:218), YLQPRTFL (SEQ ID NO:537), SETKCTLKSF (SEQ ID NO:712), TLKSFTVEK (SEQ ID NO:291), QPTESIVRF (SEQ ID NO:594), RFPNITNLCPF (SEQ ID NO:413), GEVFNATRF (SEQ ID NO:662), NATRFASVY (SEQ ID NO:595), LYNSASFSTF (SEQ ID NO:393), NSASFSTFK (SEQ ID NO:329), RQIAPGQTGK (SEQ ID NO:292), KIADYNYKL (SEQ ID NO:219), NYNYLYRLF (SEQ ID NO:394), RLFRKSNLK (SEQ ID NO:293), KPFERDISTEI (SEQ ID NO:478), YFPLQSYGF (SEQ ID NO:395), QPYRVVVL (SEQ ID NO:479), PYRVVVLSF (SEQ ID NO:396), GPKKSTNLV (SEQ ID NO:480), TSNQVAVLY (SEQ ID NO:146), VYSTGSNVF (SEQ ID NO:397), AEHVNNSY (SEQ ID NO:721), IPIGAGICASY (SEQ ID NO:596), SPRRARSVA (SEQ ID NO:481), VASQSIIAY (SEQ ID NO:597), SIIAYTMSL (SEQ ID NO:220), LGAENSVAY (SEQ ID NO:598), AYSNNSIAIPTNF (SEQ ID NO:414), IPTNFTISV (SEQ ID NO:482), TEILPVSMTK (SEQ ID NO:330), QEVFAQVKQIY (SEQ ID NO:713), KQIYKTPPIK (SEQ ID NO:294), IYKTPPIKDF (SEQ ID NO:398), LLFNKVTLA (SEQ ID NO:221), TLADAGFIK (SEQ ID NO:295), LADAGFIKQY (SEQ ID NO:147), ADAGFIKQY (SEQ ID NO:714), VLPPLLTDEMIAQY (SEQ ID NO:148), IPFAMQMAY (SEQ ID NO:599), SSTASALGK (SEQ ID NO:331), VLNDILSRL (SEQ ID NO:222), RLDKVEAEV (SEQ ID NO:223), VEAEVQIDRL (SEQ ID NO:663), AEVQIDRLI (SEQ ID NO:664), LITGRLQSL (SEQ ID NO:538), RLQSLQTYV (SEQ ID NO:224), AEIRASANL (SEQ ID NO:665), ASANLAATK (SEQ ID NO:296), HLMSFPQSA (SEQ ID NO:225), FPQSAPHGVVF (SEQ ID NO:600), APHGVVFL (SEQ ID NO:483), VTYVPAQEK (SEQ ID NO:297), TYVPAQEKNF (SEQ ID NO:399), REGVFVSNGTHW (SEQ ID NO:715), GTHWFVTQR (SEQ ID NO:332), TVYDPLQPELDSFK (SEQ ID NO:333), KEIDRLNEV (SEQ ID NO:666), QELGKYEQYIKW (SEQ ID NO:716), YEQYIKWPW (SEQ ID NO:717), QYIKWPWYI (SEQ ID NO:400), FIAGLIAIV (SEQ ID NO:226), and SEPVLKGVKL (SEQ ID NO:484).

In some cases, the peptide epitope is a SARS-CoV-2 peptide from a SARS-CoV-2-encoded membrane glycoprotein. In some cases, a peptide present in a TMMP is a peptide of from 4 amino acids (aa) to 25 aa in length (e.g., 4 aa, 5 aa, 6 aa, 7, aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa in length) of a polypeptide comprising an amino acid sequence having at least 50%, at least 60%, at least 70%, at least 75%, at least 80%, at least 85%, at least 90%, at least 95%, at least 98%, at least 99%, or 100%, amino acid sequence identity to the SARS-CoV-2 membrane glycoprotein depicted in FIG. 13D. In some cases, a peptide present in a TMMP is a peptide of from 4 amino acids (aa) to 25 aa in length (e.g., 4 aa, 5 aa, 6 aa, 7, aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa in length) of the SARS-CoV-2 membrane glycoprotein depicted in FIG. 13D.

In some cases, a peptide present in a TMMP is a SARS-CoV-2 peptide from a SARS-CoV-2-encoded membrane glycoprotein and is selected from the group consisting of: GTITVEELK (SEQ ID NO:302), EELKKLLEQW (SEQ ID NO:671), KLLEQWNLV (SEQ ID NO:155), FAYANRNRF (SEQ ID NO:544), YANRNRFLY (SEQ ID NO:545), SYFIASFRLF (SEQ ID NO:338), RLFARTRSM (SEQ ID NO:491), VPLHGTIL (SEQ ID NO:427), SELVIGAVIL (SEQ ID NO:602), HLRIAGHHL (SEQ ID NO:492), RIAGHHLGR (SEQ ID NO:233), KEITVATSRTL (SEQ ID NO:603), ATSRTLSYYK (SEQ ID NO:303), ASQRVAGDSGFAAY (SEQ ID NO:101), and VAGDSGFAAY (SEQ ID NO:102).

In some cases, the peptide epitope is a SARS-CoV-2 peptide from a SARS-CoV-2-encoded nucleocapsid phosphoprotein. In some cases, a peptide present in a TMMP is a peptide of from 4 amino acids (aa) to 25 aa in length (e.g., 4 aa, 5 aa, 6 aa, 7, aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa in length) of a polypeptide comprising an amino acid sequence having at least 50%, at least 60%, at least 70%, at least 75%, at least 80%, at least 85%, at least 90%, at least 95%, at least 98%, at least 99%, or 100%, amino acid sequence identity to the SARS-CoV-2 nucleocapsid phosphoprotein depicted in FIG. 13F. In some cases, a peptide present in a TMMP is a peptide of from 4 amino acids (aa) to 25 aa in length (e.g., 4 aa, 5 aa, 6 aa, 7, aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa in length) of the SARS-CoV-2 nucleocapsid phosphoprotein depicted in FIG. 13F.

In some cases, the peptide epitope is a SARS-CoV-2 peptide from a SARS-CoV-2-encoded nucleocapsid phosphoprotein and is selected from the group consisting of: LPNNTASWF (SEQ ID NO:567), KFPRGQGVPI (SEQ ID NO:454), NTNSSPDDQIGYY (SEQ ID NO:119), SPRWYFYYL (SEQ ID NO:455), LLLDRLNQL (SEQ ID NO:192), KAYNVTQAF (SEQ ID NO:568), QELIRQGTDYKHW (SEQ ID NO:690), ASAFFGMSR (SEQ ID NO:315), SRIGMEVTPSGTW (SEQ ID NO:691), GMEVTPSGTWL (SEQ ID NO:692), TPSGTWLTY (SEQ ID NO:569), AYKTFPPTEPK (SEQ ID NO:316), and LPAADLDDF (SEQ ID NO:570).

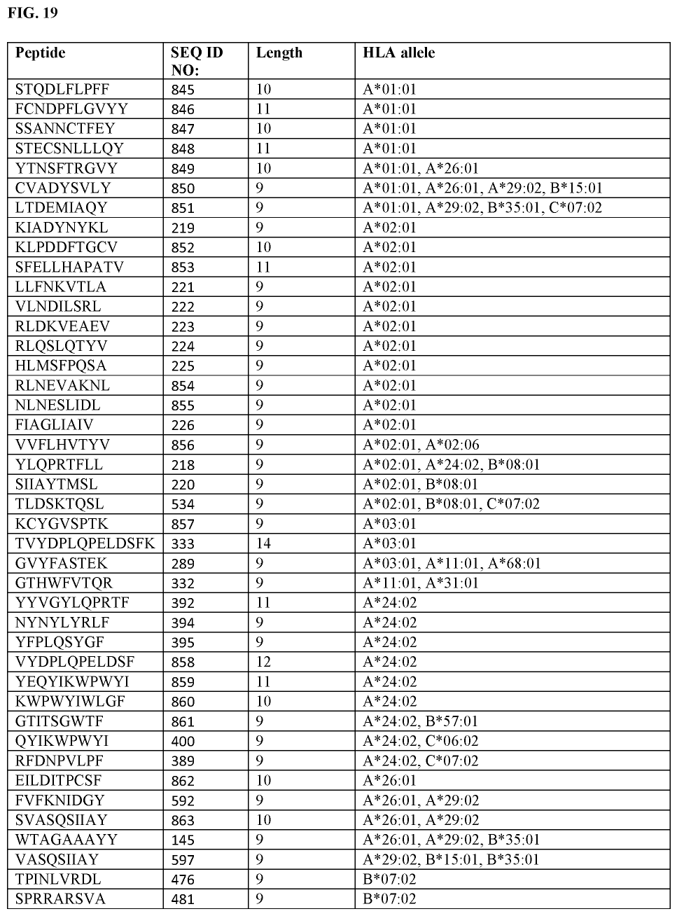

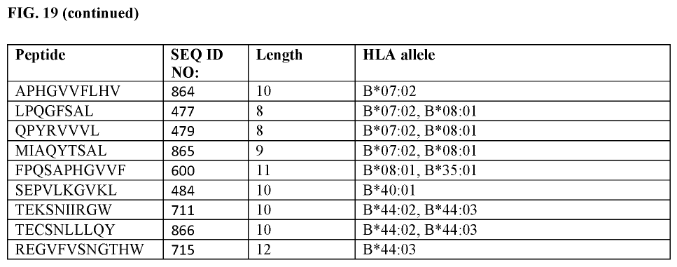

In some cases, the peptide epitope is a SARS-CoV-2 peptide depicted in FIG. 19. In some cases, the peptide is an HLA-A*01:01-restricted peptide depicted in FIG. 19. In some cases, the peptide is an HLA-A*02:01-restricted peptide depicted in FIG. 19. In some cases, the peptide is an HLA-A*11:01-restricted peptide depicted in FIG. 19. In some cases, the peptide is an HLA-A*24:01-restricted peptide depicted in FIG. 19. In some cases, the peptide is an HLA-B*07:02-restricted peptide depicted in FIG. 19. The peptides in FIG. 19 may be paired with an HLA allele as shown in FIG. 19 or a different HLA allele.

In some cases, the peptide epitope is RLQSLQTYV (SEQ ID NO:224). In some cases, the peptide epitope is YLQPRTFLL (SEQ ID NO:218).

In some cases, the peptide epitope is one that can be presented in a complex with a β2M polypeptide and an HLA-E polypeptide. As one non-limiting example, in some cases, the peptide epitope is a peptide of a SARS-CoV-2 Nsp13 (helicase) polypeptide (see, e.g., FIG. 13C). In some cases, the peptide epitope is VMPLSAPTL (SEQ ID NO:914). In some cases, a TMMP comprises an HLA-E heavy chain polypeptide and comprises the peptide epitope VMPLSAPTL (SEQ ID NO:914).

### Mhc Polypeptides

As noted above, a TMMP includes MHC polypeptides. For the purposes of the instant disclosure, the term “major histocompatibility complex (MHC) polypeptides” is meant to include MHC polypeptides of various species, including human MHC (also referred to as human leukocyte antigen (HLA)) polypeptides, rodent (e.g., mouse, rat, etc.) MHC polypeptides, and MHC polypeptides of other mammalian species (e.g., lagomorphs, non-human primates, canines, felines, ungulates (e.g., equines, bovines, ovines, caprines, etc.), and the like. The term “MHC polypeptide” is meant to include Class I MHC polypeptides (e.g., β-2 microglobulin and MHC class I heavy chain).

In some cases, the first MHC polypeptide is an MHC class I beta-2 microglobulin (β2M) polypeptide, and the second MHC polypeptide is an MHC class I heavy chain (H chain) (“MHC-H”)). In other instances, the first MHC polypeptide is an MHC class I heavy chain polypeptide; and the second MHC polypeptide is a β2M polypeptide. In some cases, both the β2M and MHC-H chain are of human origin; i.e., the MHC-H chain is an HLA heavy chain, or a variant thereof. Unless expressly stated otherwise, a TMMP does not include membrane anchoring domains (transmembrane regions) of an MHC class I heavy chain, or a part of MHC class I heavy chain sufficient to anchor the resulting TMMP to a cell (e.g., eukaryotic cell such as a mammalian cell) in which it is expressed. In some cases, the MHC class I heavy chain present in a TMMP does not include a signal peptide, a transmembrane domain, or an intracellular domain (cytoplasmic tail) associated with a native MHC class I heavy chain. Thus, e.g., in some cases, the MHC class I heavy chain present in a TMMP includes only the a1, a2, and a3 domains of an MHC class I heavy chain polypeptide. In some cases, the MHC class I heavy chain present in a TMMP has a length of from about 270 amino acids (aa) to about 290 aa. In some cases, the MHC class I heavy chain present in a TMMP has a length of 270 aa, 271 aa, 272 aa, 273 aa, 274 aa, 275 aa, 276 aa, 277 aa, 278 aa, 279 aa, 280 aa, 281 aa, 282 aa, 283 aa, 284 aa, 285 aa, 286 aa, 287 aa, 288 aa, 289 aa, or 290 aa.

### MHC Class I Heavy Chains

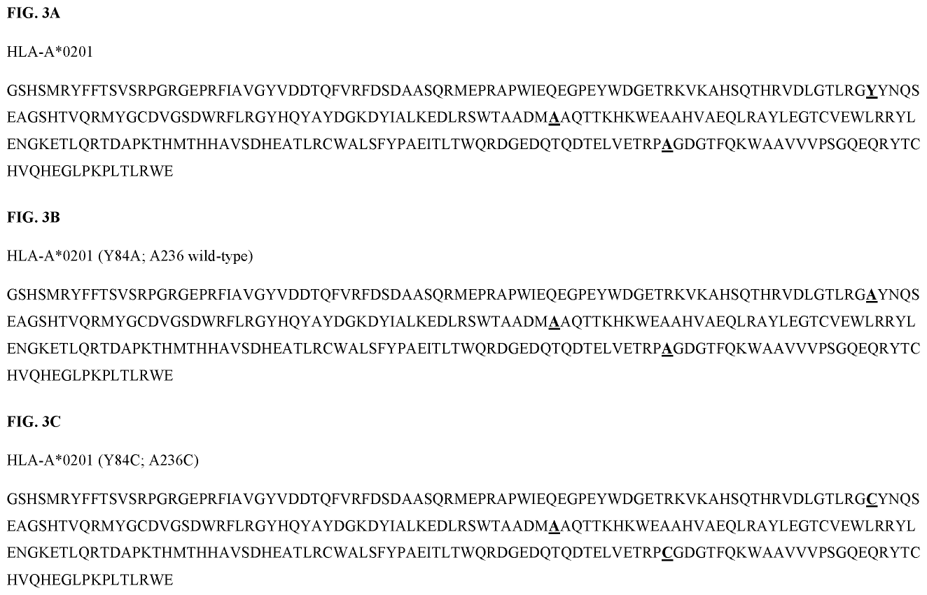

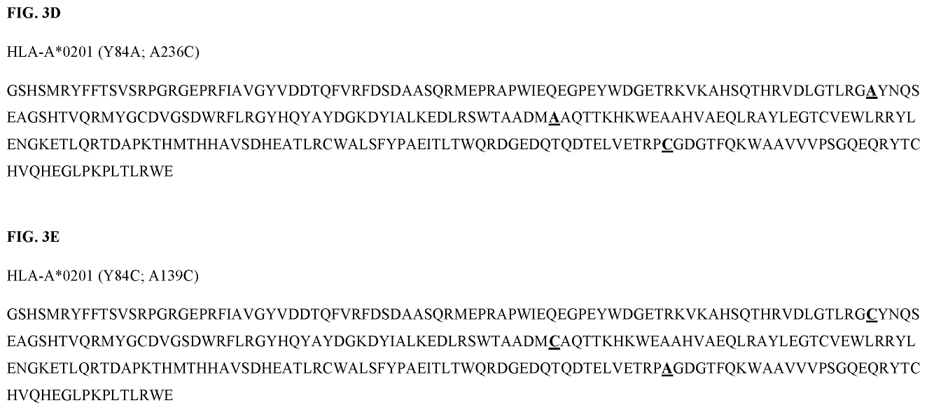

In some cases, an MHC class I heavy chain polypeptide present in a TMMP comprises an amino acid sequence having at least 75%, at least 80%, at least 85%, at least 90%, at least 95%, at least 98%, at least 99%, or 100%, amino acid sequence identity to all or part (e.g., 50, 75, 100, 150, 200, or 250 contiguous amino acids) of the amino acid sequence of any of the human HLA heavy chain polypeptides depicted in FIGS. 3-11. In some cases, the MHC class I heavy chain has a length of 270 aa, 271 aa, 272 aa, 273 aa, 274 aa, 275 aa, 276 aa, 277 aa, 278 aa, 279 aa, 280 aa, 281 aa, 282 aa, 283 aa, 284 aa, 285 aa, 286 aa, 287 aa, 288 aa, 289 aa, or 290 aa. In some cases, the MHC class I heavy chain has a length of from 270 aa to 280 aa. In some cases, an MHC class I heavy chain polypeptide present in a TMMP comprises 1-30, 1-5, 5-10, 10-15, 15-20, 20-25 or 25-30 amino acid insertions, deletions, and/or substitutions (in addition to those locations indicated as being variable in the heavy chain consensus sequences) of any one of the amino acid sequences depicted in FIG. 3-11. As noted above, the MHC class I heavy chain typically does not include transmembrane or cytoplasmic domains.

In some cases, an MHC polypeptide of a TMMP is a human MHC polypeptide, where human MHC polypeptides are also referred to as “human leukocyte antigen” (“HLA”) polypeptides. In some cases, an MHC polypeptide of a TMMP is a Class I HLA polypeptide, e.g., a β2-microglobulin polypeptide, or a Class I HLA heavy chain polypeptide. Class I HLA heavy chain polypeptides include HLA-A heavy chain polypeptides, HLA-B heavy chain polypeptides, HLA-C heavy chain polypeptides, HLA-E heavy chain polypeptides, HLA-F heavy chain polypeptides, and HLA-G heavy chain polypeptides.

In some cases, a TMMP comprises an HLA-A heavy chain polypeptide. The HLA-A heavy chain peptide sequences, or portions thereof, that may be that may be incorporated into a TMMP include, but are not limited to, the alleles: A*0101, A*0201, A*0301, A*1101, A*2301, A*2402, A*2407, A*3303, and A*3401. Any of those alleles may comprise a mutation at one or more of positions 84, 139, and 236 (as shown in FIG. 3-6 selected from: a tyrosine to alanine at position 84 (Y84A); a tyrosine to cysteine at position 84 (Y84C); an alanine to cysteine at position 139 (A139C); and an alanine to cysteine substitution at position 236 (A236C). In addition, HLA-A sequence having at least 75% (e.g., at least 80%, at least 85%, at least 90%, at least 95%, at least 98%, at least 99%) or 100% amino acid sequence identity to all or part (e.g., 50, 75, 100, 150, 200, or 250 contiguous amino acids) of the sequence of those HLA-A alleles may also be employed (e.g., it may comprise 1-25, 1-5, 5-10, 10-15, 15-20, 20-25, or 25-30 amino acid insertions, deletions, and/or substitutions). Some examples are provided below.

### HLA-A02

In some cases, an MHC class I heavy chain polypeptide of a TMMP can comprise an amino acid sequence having at least 75%, at least 80%, at least 85%, at least 90%, at least 95%, at least 98%, at least 99%, or 100%, amino acid sequence identity to the amino acid sequence depicted in FIG. 3A. In some cases, an HLA-A heavy chain polypeptide suitable for inclusion in a TMMP comprises the amino acid sequence depicted in FIG. 3A. This HLA-A heavy chain polypeptide is also referred to as “HLA-A*0201” or simply “HLA-A02.” As described in WO 2018/119114 and WO 2020/132138, TMMPs can comprise one or more mutations from the wild-type HLA-A02, including to provide Cys residues that can form disulfide bonds, e.g., (i) between the β2M and MHC class I heavy chain, and/or (ii) between the MHC heavy chain and a linker that joins the peptide epitope to the β2M polypeptide, and/or (iii) an intrachain disulfide bond within the MHC class I heavy chain polypeptide.

To facilitate the formation of such disulfide bonds, one or more non-naturally occurring Cys residues can be provided in the heavy chain polypeptide. For example, the MHC class I heavy chain polypeptide comprises an amino acid sequence having at least 75%, at least 80%, at least 85%, at least 90%, at least 95%, at least 98%, at least 99%, or 100%, amino acid sequence identity to A) the HLA-A02 (Y84C; A236C) amino acid sequence depicted in FIG. 3C, where amino acid 84 is a Cys, and where amino acid 236 is a Cys. In some cases, the Cys-236 forms an interchain disulfide bond with Cys-12 of a variant β2M polypeptide that comprises an R12C substitution; or
B) the HLA-A02 (Y84A; A236C) amino acid sequence: depicted in FIG. 3D, where amino acid 84 is Ala and amino acid 236 is Cys. In some cases, the Cys-236 forms an interchain disulfide bond with Cys-12 of a variant β2M polypeptide that comprises an R12C substitution; or C) the HLA-A02 (Y84C; A139C) amino acid sequence depicted in FIG. 3E, where amino acid 84 is Cys and amino acid 139 is Cys.

As noted above, in some cases the MHC class I heavy chain polypeptide comprises an amino acid sequence having at least 75%, at least 80%, at least 85%, at least 90%, at least 95%, at least 98%, at least 99%, or 100%, amino acid sequence identity to the HLA-A02 (Y84A; A236 wild-type) amino acid sequence depicted in FIG. 3B, where amino acid 84 is a Tyr, and amino acid 236 is an Ala.

### HLA-A11 (HLA-A*1101)

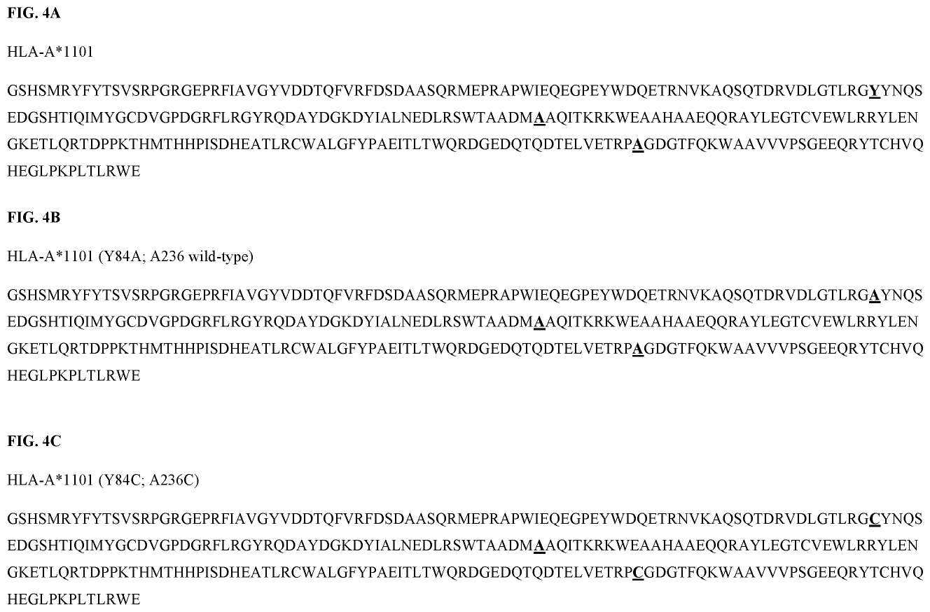

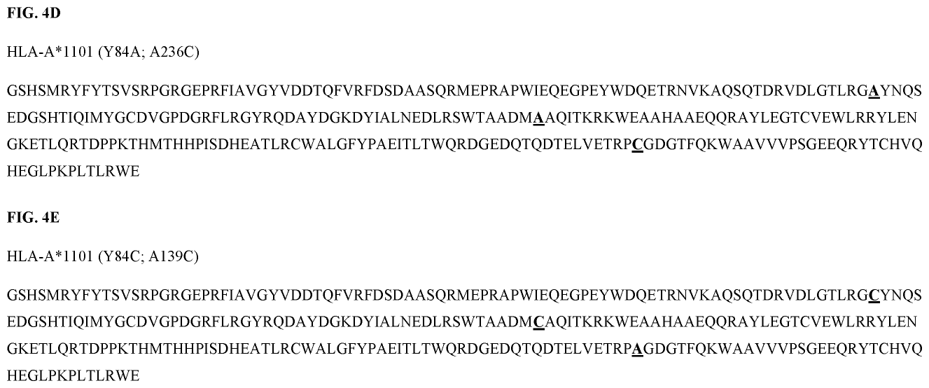

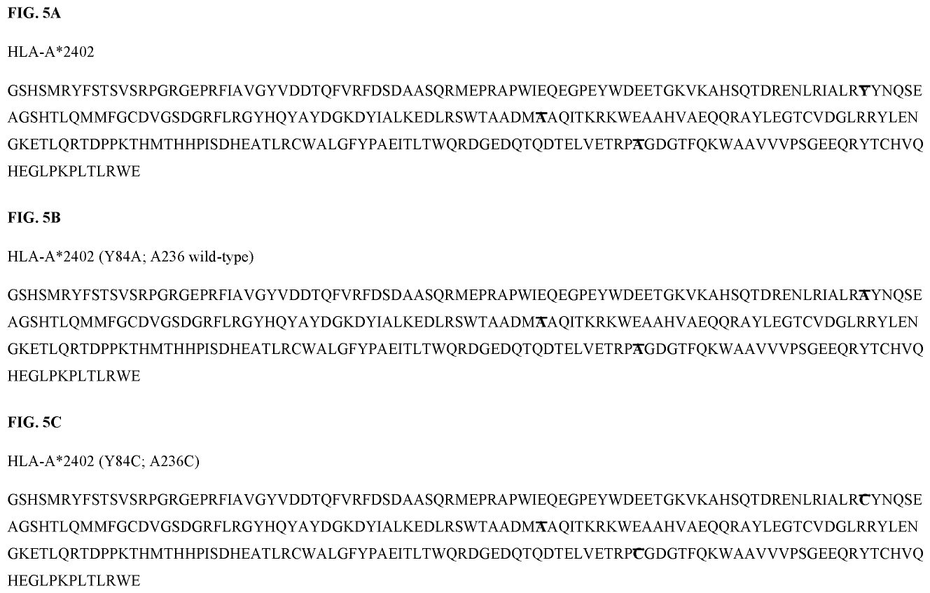

In some cases, an MHC class I heavy chain polypeptide of a TMMP can comprise an amino acid sequence having at least 75%, at least 80%, at least 85%, at least 90%, at least 95%, at least 98%, at least 99%, or 100%, amino acid sequence identity to the amino acid sequence depicted in FIG. 4A. In some cases, an HLA-A heavy chain polypeptide suitable for inclusion in a TMMP comprises the amino acid sequence depicted in FIG. 4A. This HLA-A heavy chain polypeptide is also referred to as “HLA-A*1101” or simply “HLA-A11.” Variants can include, e.g., the MHC class I heavy chain polypeptide comprises an amino acid sequence having at least 75%, at least 80%, at least 85%, at least 90%, at least 95%, at least 98%, at least 99%, or 100%, amino acid sequence identity to:
A) the HLA-A11 (Y84A; A236 wild-type) amino acid sequence depicted in FIG. 4B, where amino acid 84 is a Tyr, and amino acid 236 is an Ala;
B) the HLA-A11 (Y84C; A236C) amino acid sequence depicted in FIG. 4C, where amino acid 84 is a Cys, and where amino acid 236 is a Cys;
C) the HLA-A11 (Y84A; A236C) amino acid sequence: depicted in FIG. 4D, where amino acid 84 is Ala and amino acid 236 is Cys. In some cases, the Cys-236 forms an interchain disulfide bond with Cys-12 of a variant β2M polypeptide that comprises an R12C substitution; and D) the HLA-A11 (Y84C; A139C) amino acid sequence depicted in FIG. 4E, where amino acid 84 is Cys and amino acid 139 is Cys. In some cases, Cys-84 forms an intrachain disulfide bond with Cys-139.

### HLA-A24 (HLA-A*2402)

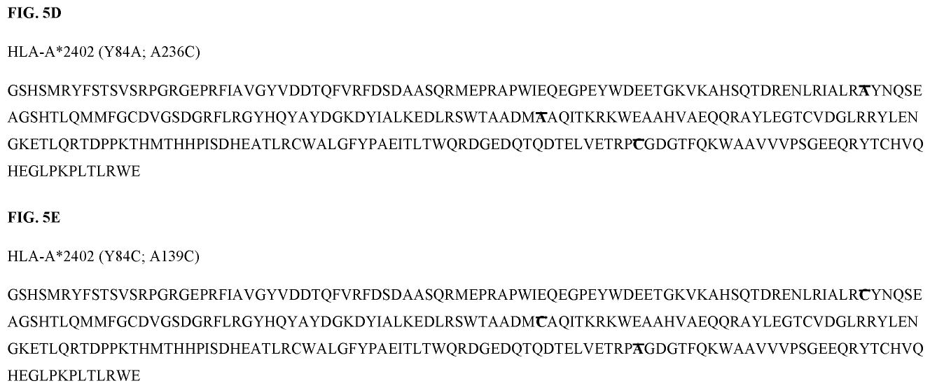

In some cases, an MHC class I heavy chain polypeptide of a TMMP can comprise an amino acid sequence having at least 75%, at least 80%, at least 85%, at least 90%, at least 95%, at least 98%, at least 99%, or 100%, amino acid sequence identity to the amino acid sequence depicted in FIG. 5A. In some cases, an HLA-A heavy chain polypeptide suitable for inclusion in a TMMP comprises the amino acid sequence depicted in FIG. 5A. This HLA-A heavy chain polypeptide is also referred to as “HLA-A*2402” or simply “HLA-A24.” Variants can include, e.g., the MHC class I heavy chain polypeptide comprises an amino acid sequence having at least 75%, at least 80%, at least 85%, at least 90%, at least 95%, at least 98%, at least 99%, or 100%, amino acid sequence identity to:
A) the HLA-A24 (Y84A; A236 wild-type) amino acid sequence depicted in FIG. 5B, where amino acid 84 is a Tyr, and amino acid 236 is an Ala;
B) the HLA-A24 (Y84C; A236C) amino acid sequence depicted in FIG. 5C, where amino acid 84 is a Cys, and where amino acid 236 is a Cys;
C) the HLA-A24 (Y84A; A236C) amino acid sequence: depicted in FIG. 5D, where amino acid 84 is Ala and amino acid 236 is Cys. In some cases, the Cys-236 forms an interchain disulfide bond with Cys-12 of a variant β2M polypeptide that comprises an R12C substitution; and
D) the HLA-A24 (Y84C; A139C) amino acid sequence depicted in FIG. 5E, where amino acid 84 is Cys and amino acid 139 is Cys. In some cases, Cys-84 forms an intrachain disulfide bond with Cys-139.

### HLA-A33 (HLA-A*3303)

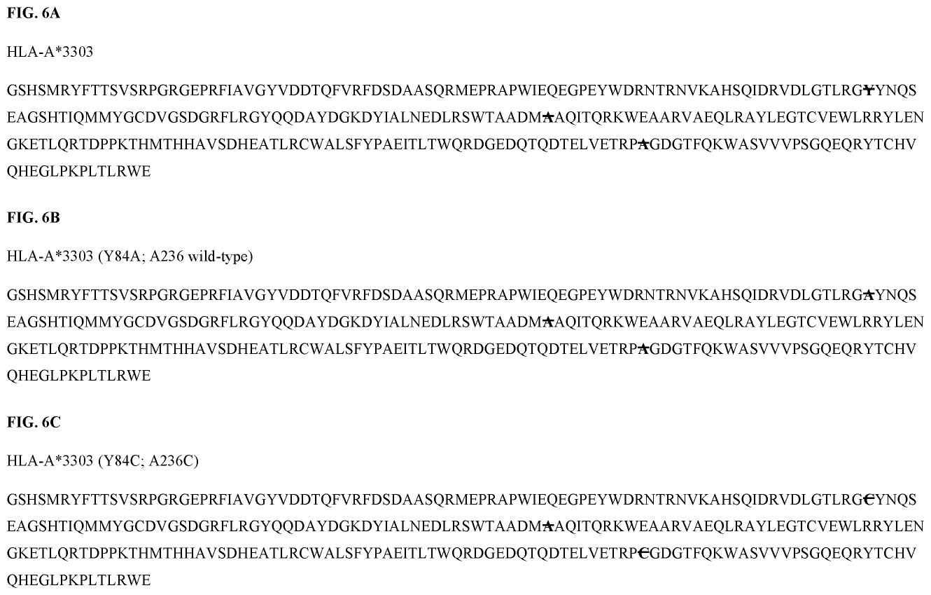

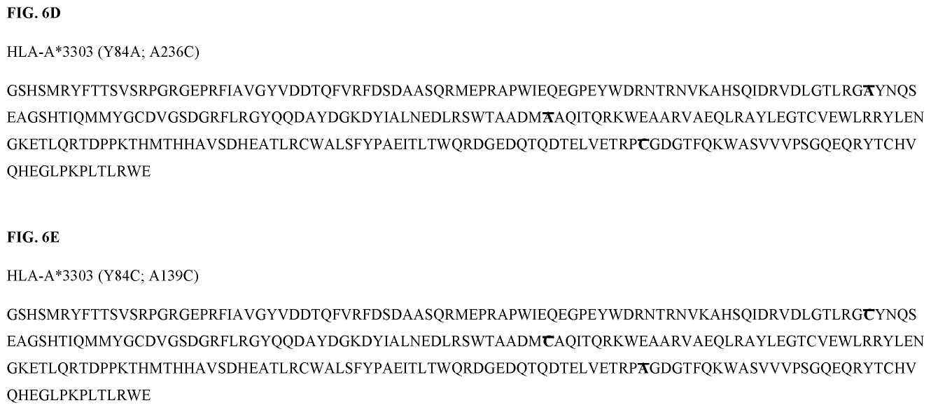

In some cases, an MHC class I heavy chain polypeptide of a TMMP can comprise an amino acid sequence having at least 75%, at least 80%, at least 85%, at least 90%, at least 95%, at least 98%, at least 99%, or 100%, amino acid sequence identity to the amino acid sequence depicted in FIG. 6A. In some cases, an HLA-A heavy chain polypeptide suitable for inclusion in a TMMP comprises the amino acid sequence depicted in FIG. 6A. This HLA-A heavy chain polypeptide is also referred to as “HLA-A*3303” or simply “HLA-A33.” Variants can include, e.g., the MHC class I heavy chain polypeptide comprises an amino acid sequence having at least 75%, at least 80%, at least 85%, at least 90%, at least 95%, at least 98%, at least 99%, or 100%, amino acid sequence identity to:
A) the HLA-A24 (Y84A; A236 wild-type) amino acid sequence depicted in FIG. 6B, where amino acid 84 is a Tyr, and amino acid 236 is an Ala;
B) the HLA-A24 (Y84C; A236C) amino acid sequence depicted in FIG. 6C, where amino acid 84 is a Cys, and where amino acid 236 is a Cys;
C) the HLA-A24 (Y84A; A236C) amino acid sequence: depicted in FIG. 6D, where amino acid 84 is Ala and amino acid 236 is Cys. In some cases, the Cys-236 forms an interchain disulfide bond with Cys-12 of a variant β2M polypeptide that comprises an R12C substitution; and
D) the HLA-A24 (Y84C; A139C) amino acid sequence depicted in FIG. 6E, where amino acid 84 is Cys and amino acid 139 is Cys. In some cases, Cys-84 forms an intrachain disulfide bond with Cys-139.

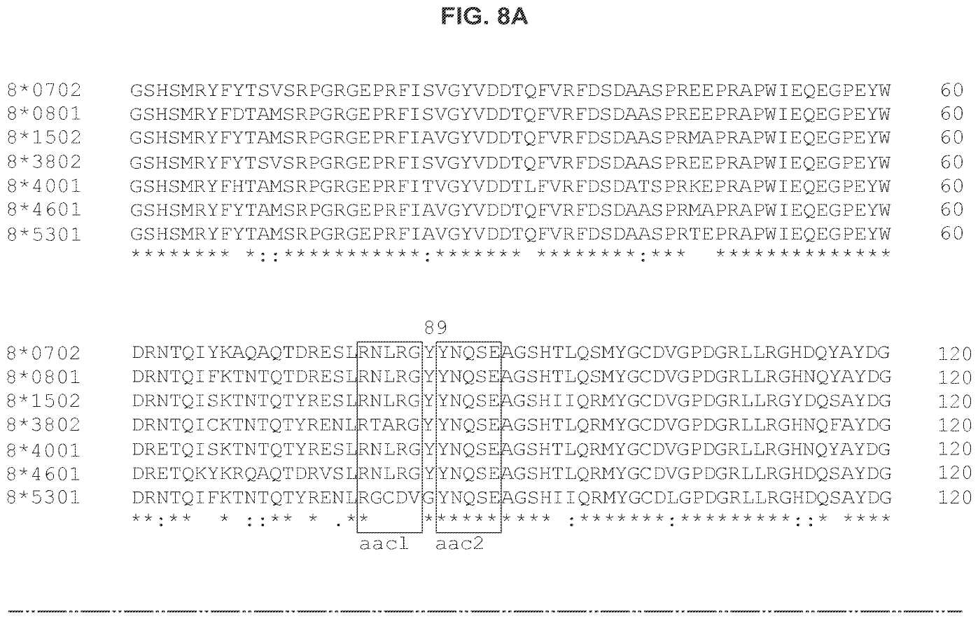

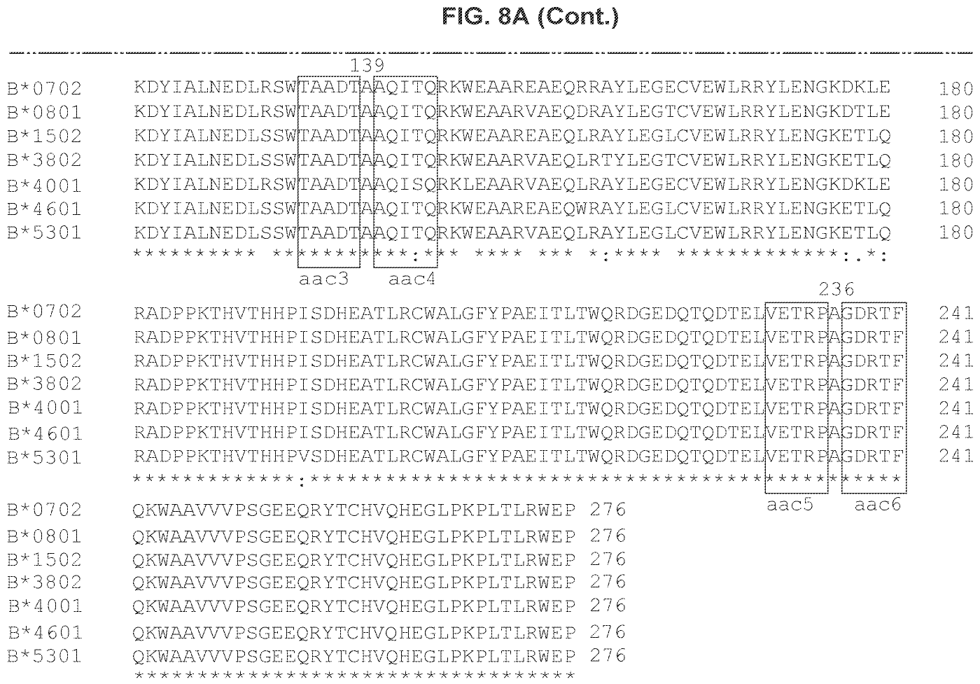

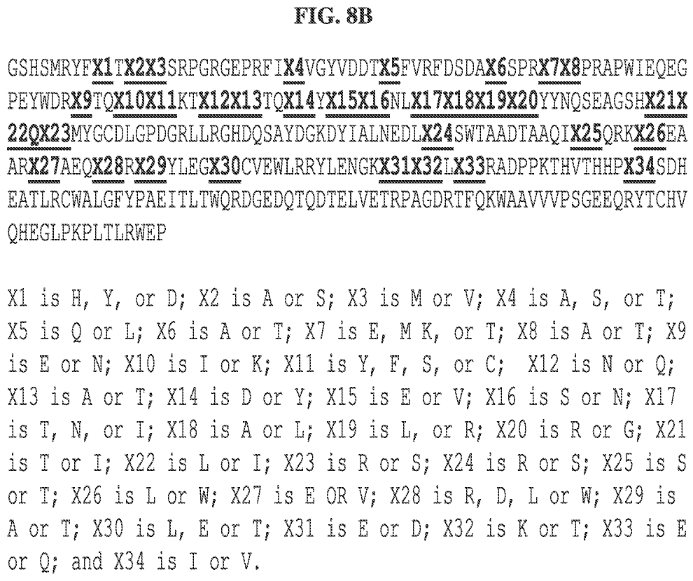

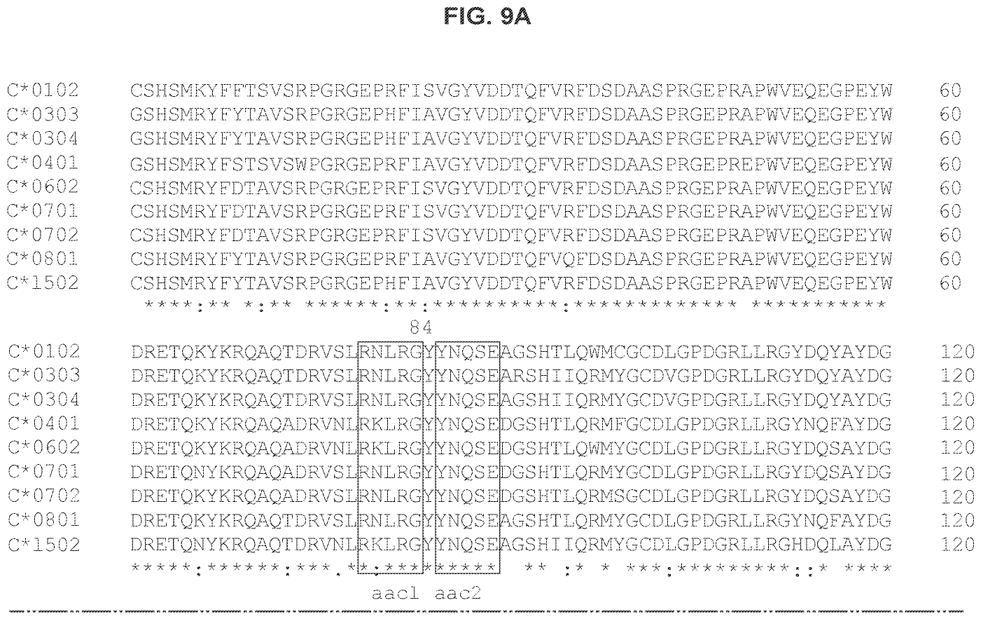

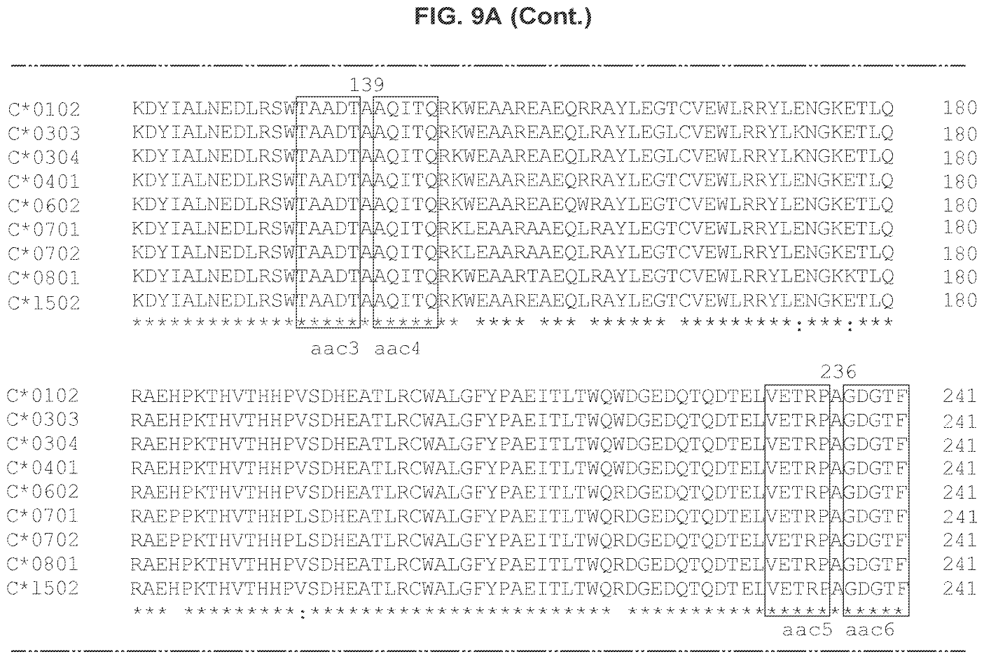

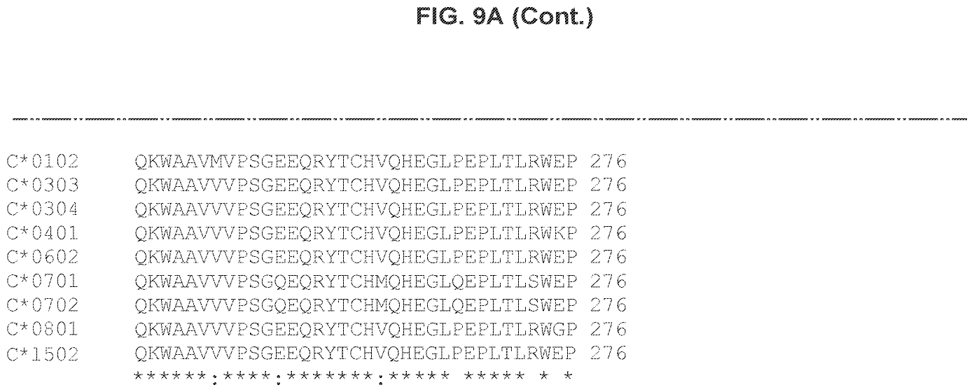

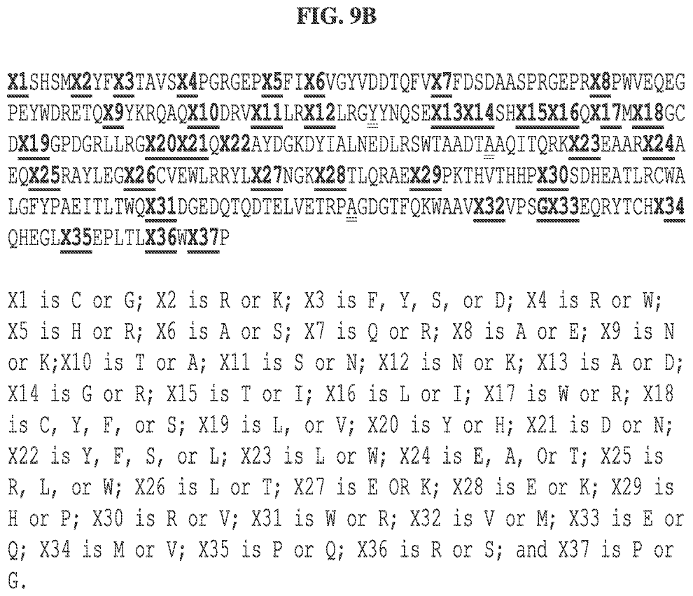

FIGS. 7-9 provide alignments of mature HLA class I heavy chain amino acid sequences (without leader sequences or transmembrane domains or intracellular domains). The aligned amino acid sequences in FIG. 7A are HLA-A class I heavy chains of the following alleles: A*0101, A*0201, A*0301, A*1101, A*2301, A*2402, A*2407, A*3303, and A*3401. The aligned amino acid sequences in FIG. 8A are HLA-B class I heavy chains of the following alleles: B*0702, B*0801, B*1502, B*3802, B*4001, B*4601, and B*5301. The aligned amino acid sequences in FIG. 9A are HLA-C class I heavy chains of the following alleles: C*0102, C*0303, C*0304, C*0401, C*0602, C*0701, C*0801, and C*1502. Indicated in the alignments are the locations (84 and 139 of the mature proteins) where cysteine residues may be introduced (e.g., by substitution) for the formation of a disulfide bond to stabilize the HLA H chain—β2M complex. Also shown in the alignment is position 236 (of the mature polypeptide), which may be substituted by a cysteine residue that can form an inter-chain disulfide bond with β2M (e.g., at aa 12). The boxes flanking residues 84, 139 and 236 show the groups of five amino acids on either sides of those six sets of five residues, denoted aacl (for “amino acid cluster 1”), aac2 (for “amino acid cluster 2”), aac3 (for “amino acid cluster 3”), aac4 (for “amino acid cluster 4”), aac5 (for “amino acid cluster 5”), and aac6 (for “amino acid cluster 6”), that may be replaced by 1 to 5 amino acids selected independently from (i) any naturally occurring amino acid or (ii) any naturally occurring amino acid except proline or glycine.

FIGS. 7A, 8A, and 9A provide alignments of the amino acid sequences of mature HLA-A, -B, and -C class I heavy chains, respectively. The sequences are provided for the extracellular portion of the mature protein (without leader sequences or transmembrane domains or intracellular domains). The positions of aa residues 84, 139, and 236 and their flanking residues (aac1 to aac6) that may be replaced by 1 to 5 amino acids selected independently from (i) any naturally occurring amino acid or (ii) any naturally occurring amino acid except proline or glycine ae also shown. FIGS. 7B, 8B, and 9B provide consensus amino acid sequences for the HLA-A, -B, and -C sequences, respectively, provide in FIGS. 7A, 8A, and 9A. The consensus sequences show the variable amino acid positions as “X” residues sequentially numbered and the locations of amino acids 84, 139 and 236 double underlined.

With regard to FIG. 7A, in some cases: i) aac1 (amino acid cluster 1) may be the amino acid sequence GTLRG (SEQ ID NO:915) or that sequence with one or two amino acids deleted or substituted with other naturally occurring amino acids (e.g., L replaced by I, V, A or F); ii) aac2 (amino acid cluster 2) may be the amino acid sequence YNQSE (SEQ ID NO:916) or that sequence with one or two amino acids deleted or substituted with other naturally occurring amino acids (e.g., N replaced by Q, Q replaced by N, and/or E replaced by D); iii) aac3 (amino acid cluster 3) may be the amino acid sequence TAADM (SEQ ID NO:917) or that sequence with one or two amino acids deleted or substituted with other naturally occurring amino acids (e.g., T replaced by S, A replaced by G, D replaced by E, and/or M replaced by L, V, or I); iv) aac4 (amino acid cluster 4) may be the amino acid sequence AQTTK (SEQ ID NO:918) or that sequence with one or two amino acids deleted or substituted with other naturally occurring amino acids (e.g., A replaced by G, Q replaced by N, or T replaced by S, and or K replaced by R or Q); v) aac5 (amino acid cluster 5) may be the amino acid sequence VETRP (SEQ ID NO:919) or that sequence with one or two amino acids deleted or substituted with other naturally occurring amino acids (e.g., V replaced by I or L, E replaced by D, T replaced by S, and/or R replaced by K); and/or vi) aac6 (amino acid cluster 6) may be the amino acid sequence GDGTF (SEQ ID NO:920) or that sequence with one or two amino acids deleted or substituted with other naturally occurring amino acids (e.g., D replaced by E, T replaced by S, or F replaced by L, W, or Y).

With regard to FIG. 8A, in some cases: i) aac1 (amino acid cluster 1) may be the amino acid sequence RNLRG (SEQ ID NO:921) or that sequence with one or two amino acids deleted or substituted with other naturally occurring amino acids (e.g., N replaced by T or I; and/or L replaced by A; and/or the second R replaced by L; and/or the G replaced by R); ii) aac2 (amino acid cluster 2) may be the amino acid sequence YNQSE (SEQ ID NO:916) or that sequence with one or two amino acids deleted or substituted with other naturally occurring amino acids (e.g., N replaced by Q, Q replaced by N, and/or E replaced by D); iii) aac3 (amino acid cluster 3) may be the amino acid sequence TAADT (SEQ ID NO:922) or that sequence with one or two amino acids deleted or substituted with other naturally occurring amino acids (e.g., the first T replaced by S; and/or A replaced by G; and/or D replaced by E; and/or the second T replaced by S); iv) aac4 (amino acid cluster 4) may be the amino acid sequence AQITQ (SEQ ID NO:923) or that sequence with one or two amino acids deleted or substituted with other naturally occurring amino acids (e.g., A replaced by G; and/or the first Q replaced by N; and/or I replaced by L or V; and/or the T replaced by S; and/or the second Q replaced by N); v) aac5 (amino acid cluster 5) may be the amino acid sequence VETRP (SEQ ID NO:919) or that sequence with one or two amino acids deleted or substituted with other naturally occurring amino acids (e.g., V replaced by I or L, E replaced by D, T replaced by S, and/or R replaced by K); and/or vi) aac6 (amino acid cluster 6) may be the amino acid sequence GDRTF (SEQ ID NO:924) or that sequence with one or two amino acids deleted or substituted with other naturally occurring amino acids (e.g., D replaced by E; and/or T replaced by S; and/or R replaced by K or H; and/or F replaced by L, W, or Y).

With regard to FIG. 9A, in some cases: i) aac1 (amino acid cluster 1) may be the amino acid sequence RNLRG (SEQ ID NO:921) or that sequence with one or two amino acids deleted or substituted with other naturally occurring amino acids (e.g., N replaced by K; and/or L replaced by A or I; and/or the second R replaced by H; and/or the G replaced by T or S); ii) aac2 (amino acid cluster 2) may be the amino acid sequence YNQSE (SEQ ID NO:916) or that sequence with one or two amino acids deleted or substituted with other naturally occurring amino acids (e.g., N replaced by Q, Q replaced by N, and/or E replaced by D); iii) aac3 (amino acid cluster 3) may be the amino acid sequence TAADT (SEQ ID NO:922) or that sequence with one or two amino acids deleted or substituted with other naturally occurring amino acids (e.g., the first T replaced by S; and/or A replaced by G; and/or D replaced by E; and/or the second T replaced by S); iv) aac4 (amino acid cluster 4) may be the amino acid sequence AQITQ (SEQ ID NO:923) or that sequence with one or two amino acids deleted or substituted with other naturally occurring amino acids (e.g., A replaced by G; and/or the first Q replaced by N; and/or I replaced by L; and/or the second Q replaced by N or K); v) aac5 (amino acid cluster 5) may be the amino acid sequence VETRP (SEQ ID NO:919) or that sequence with one or two amino acids deleted or substituted with other naturally occurring amino acids (e.g., V replaced by I or L, E replaced by D, T replaced by S, and/or R replaced by K or H); and/or vi) aac6 (amino acid cluster 6) may be the amino acid sequence GDGTF (SEQ ID NO:920) or that sequence with one or two amino acids deleted or substituted with other naturally occurring amino acids (e.g., D replaced by E; and/or T replaced by S; and/or F replaced by L, W, or Y).

### Non-Classical HLA-E, -F, and -G MHC Class I Heavy Chains

In some cases, a TMMP comprises a non-classical MHC class I heavy chain polypeptide. Among the non-classical HLA heavy chain polypeptides, or portions thereof, that may be that may be incorporated into a TMMP include, but are not limited to, those of HLA-E, -F, and -G alleles. Amino acid sequences for HLA-E, -F, and -G heavy chain polypeptides, (and the HLA-A, B and C alleles) may be found on the world wide web hla.alleles.org/nomenclature/index.html, the European Bioinformatics Institute (www(dot)ebi(dot)ac(dot)uk), which is part of the European Molecular Biology Laboratory(EMBL), and at the National Center for Biotechnology Information (www(dot)ncbi(dot)nlm(dot)nih(dot)gov).

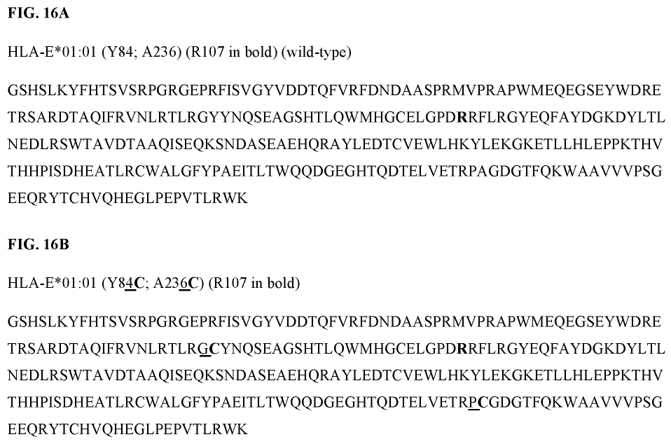

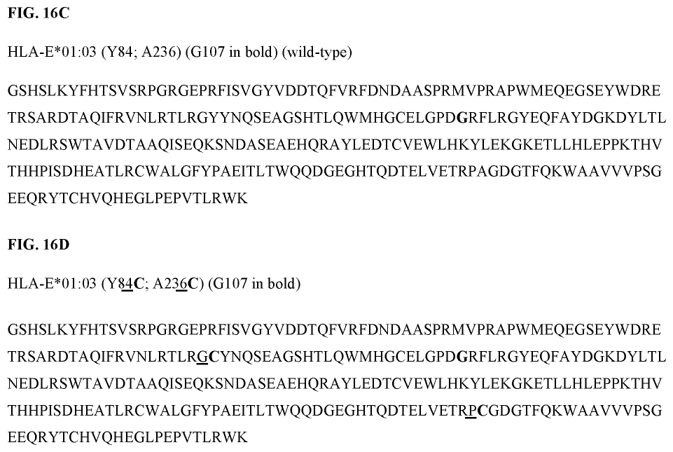

Non-limiting examples of suitable HLA-E alleles include, but are not limited to, HLA-E*0101 (HLA-E*01:01:01:01), HLA-E*01:03(HLA-E*01:03:01:01), HLA-E*01:04, HLA-E*01:05, HLA-E*01:06, HLA-E*01:07, HLA-E*01:09, and HLA-E*01:10. Of these, isoforms HLA-E*0101 and HLA-E*01.03 are of particular note since these are highly prevalent alleles, and differ by only 1 amino acid (Arg or Gly at position 107). For example, amino acid sequences of suitable HLA-E heavy chain polypeptides are provided in FIG. 16A-16D, where FIG. 16A provides the amino acid sequence of HLA-E*01:01 (wild-type); FIG. 16B provides the amino acid sequence of HLA-E*01:01 with Y84C and A2346C substitutions; FIG. 16C provides the amino acid sequence of HLA-E*01:03 (wild-type); and FIG. 16D provides the amino acid sequence of HLA-E*01:03 with Y84C and A2346C substitutions. In some cases, therefore, an MHC class I heavy chain polypeptide of a TMMP can comprise an amino acid sequence having at least 75%, at least 80%, at least 85%, at least 90%, at least 95%, at least 98%, at least 99%, or 100%, amino acid sequence identity to the amino acid sequence depicted in FIG. 16A, 16B, 16C or 16D.

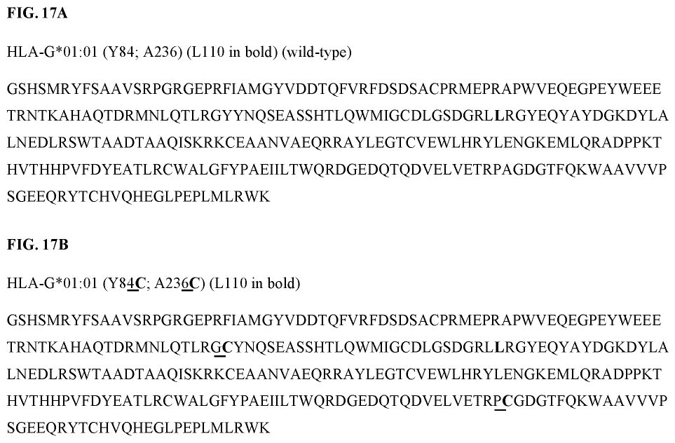

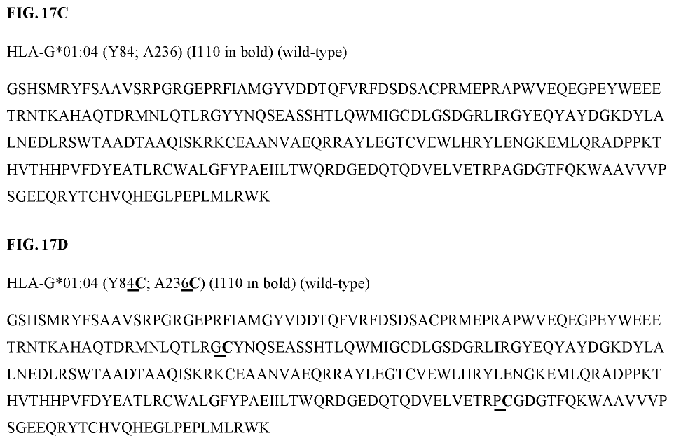

Non-limiting examples of suitable HLA-F alleles include, but are not limited to, HLA-F*0101 (HLA-F*01:01:01:01), HLA-F*01:02, HLA-F*01:03(HLA-F*01:03:01:01), HLA-F*01:04, HLA-F*01:05, and HLA-F*01:06. Non-limiting examples of suitable HLA-G alleles include, but are not limited to, HLA-G*0101 (HLA-G*01:01:01:01), HLA-G*01:02, HLA-G*01:03(HLA-G*01:03:01:01), HLA-G*01:04 (HLA-G*01:04:01:01), HLA-G*01:06, HLA-G*01:07, HLA-G*01:08, HLA-G*01:09: HLA-G*01:10, HLA-G*01:10, HLA-G*01:11, HLA-G*01:12, HLA-G*01:14, HLA-G*01:15, HLA-G*01:16, HLA-G*01:17, HLA-G*01:18: HLA-G*01:19, HLA-G*01:20, and HLA-G*01:22. Of these, isoforms HLA-G*0101 (HLA-G*01:01:01:01) and HLA-G*01:04 (HLA-G*01:04:01:01) are of particular note since these are highly prevalent alleles. For example, amino acid sequences of suitable HLA-G heavy chain polypeptides are provided in FIG. 17A-17D, where FIG. 17A provides the amino acid sequence of HLA-G*01:01 (wild-type); FIG. 17B provides the amino acid sequence of HLA-G*01:01 with Y84C and A2346C substitutions; FIG. 17C provides the amino acid sequence of HLA-G*01:04 (wild-type); and FIG. 17D provides the amino acid sequence of HLA-G*01:04 with Y84C and A2346C substitutions. In some cases, therefore, an MHC class I heavy chain polypeptide of a TMMP can comprise an amino acid sequence having at least 75%, at least 80%, at least 85%, at least 90%, at least 95%, at least 98%, at least 99%, or 100%, amino acid sequence identity to the amino acid sequence depicted in FIG. 17A, 17B, 17C or 17D.

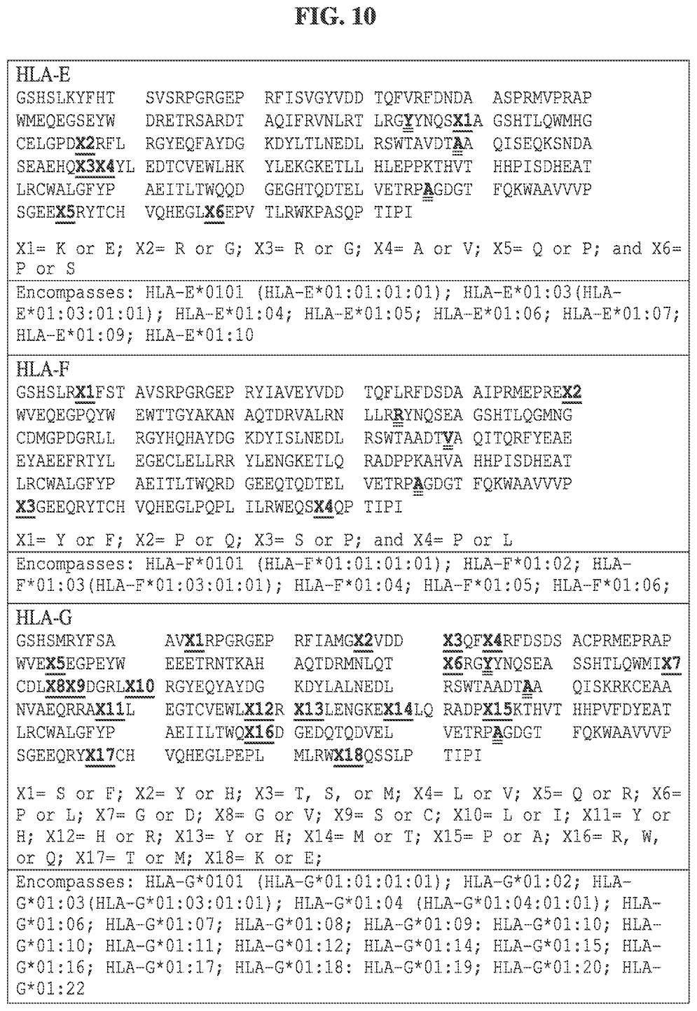

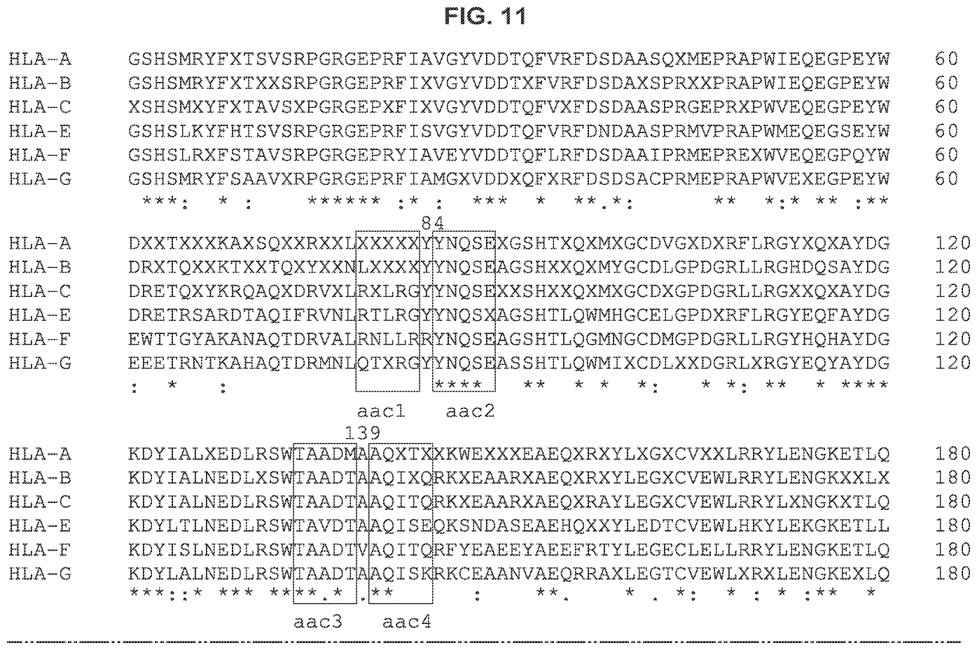

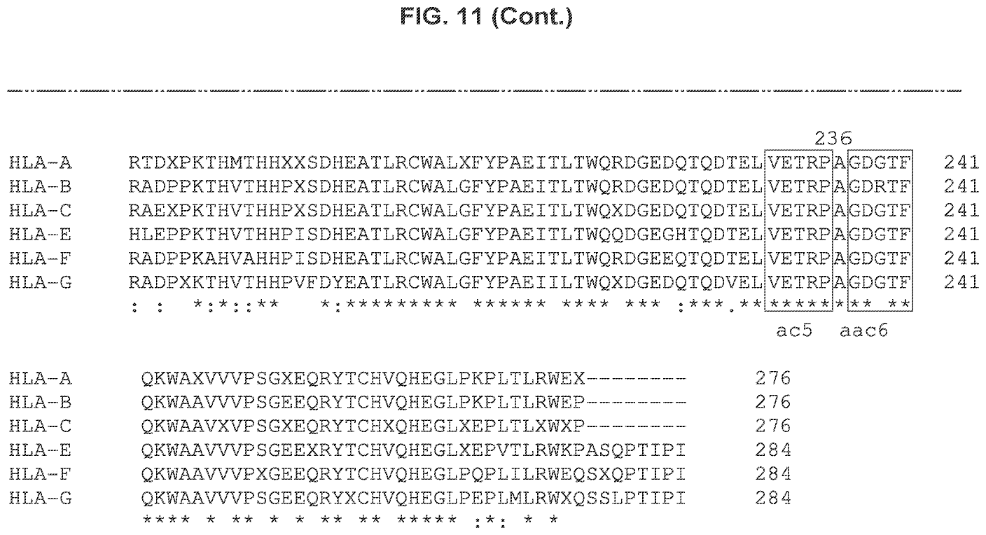

Consensus sequences for those HLA E, -F and -G alleles without all, or substantially all, of the leader, transmembrane and cytoplasmic sequences are provided in FIG. 10, and aligned with consensus sequences of the above-mentioned HLA-A, -B and -C alleles in FIG. 11.

FIG. 10 provides a consensus sequence for each of HLA-E, -F, and -G with the variable aa positions indicated as “X” residues sequentially numbered and the locations of aas 84, 139 and 236 double underlined.

FIG. 11 provides an alignment of the consensus amino acid sequences for HLA-A, -B, -C, -E, -F, and -G, which are given in FIGS. 7-11. Variable residues in each sequence are listed as “X” with the sequential numbering removed. The locations of aas 84, 139 and 236 are indicated with their flanking five-amino acid clusters that may be replaced by 1 to 5 amino acids selected independently from (i) any naturally occurring amino acid or (ii) any naturally occurring amino acid except proline or glycine are also shown.

Any of the above-mentioned HLA-E, -F, and/or -G alleles may comprise a substitution at one or more of positions 84, 139 and/or 236 as shown in FIG. 11 for the consensus sequences. In some cases, the substitutions may be selected from a: position 84 tyrosine to alanine (Y84A) or cysteine (Y84C), or, in the case of HLA-F, an R84A or R84C substitution; a position 139 alanine to cysteine (A139C), or, in the case of HLA-F, a V139C; and an alanine to cysteine substitution at position 236 (A236C). In addition, an HLA-E, -F and/or -G sequence having at least 75% (e.g., at least 80%, at least 85%, at least 90%, at least 95%, at least 98%, at least 99%) or 100% amino acid sequence identity to all or part (e.g., 50, 75, 100, 150, 200, or 250 contiguous amino acids) of their respective consensus sequences set forth in FIG. 11 may also be employed (e.g., the sequences may comprise 1-25, 1-5, 5-10, 10-15, 15-20, 20-25, or 25-30 amino acid insertions, deletions, and/or substitutions in addition to changes at variable residues listed therein).

### Beta-2 Microglobulin

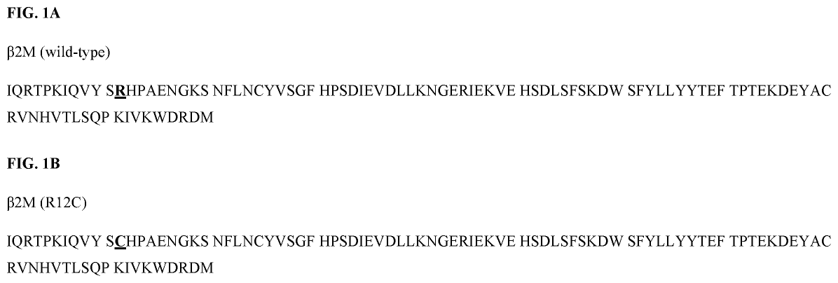

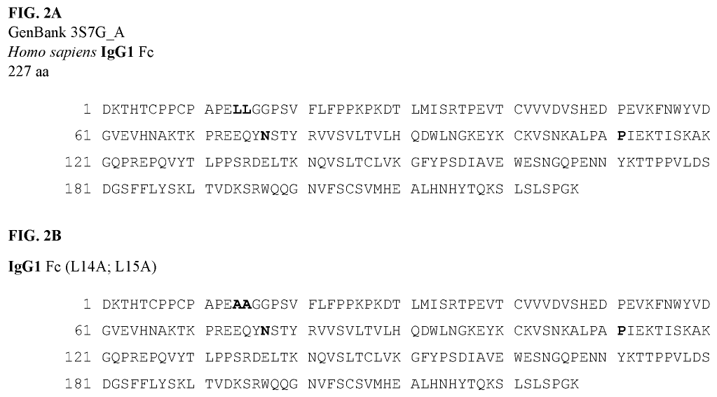

A β2-microglobulin (β2M) polypeptide of a TMMP can be a human β2M polypeptide, a non-human primate β2M polypeptide, a murine β2M polypeptide, and the like. In some instances, a β2M polypeptide comprises an amino acid sequence having at least 75%, at least 80%, at least 85%, at least 90%, at least 95%, at least 98%, at least 99%, or 100%, amino acid sequence identity to the amino acid sequence depicted in FIG. 1A (wild-type human β2M). In some cases, a β2M polypeptide present in a TMMP comprises the amino acid sequence: depicted in FIG. 1A (wild-type human β2M).

In some cases, an MHC polypeptide present in a TMMP comprises a single amino acid substitution relative to a reference MHC polypeptide (where a reference MHC polypeptide can be a wild-type MHC polypeptide), where the single amino acid substitution substitutes an amino acid with a cysteine (Cys) residue. Such cysteine residues can form a disulfide bond with a naturally occurring or non-naturally occurring cysteine residue present in the MHC heavy chain of the TMMP. As used herein, a reference to a “non-naturally occurring Cys residue” in an MHC class I polypeptide means that the polypeptide comprises a Cys residue in a location where there is no Cys in the corresponding wild-type polypeptide. This can be accomplished through routine protein engineering in which a cysteine is substituted for the amino acid that occurs in the wild-type sequence.

In some cases, a β2M polypeptide present in a TMMP comprises an amino acid sequence having at least 75%, at least 80%, at least 85%, at least 90%, at least 95%, at least 98%, at least 99%, or 100%, amino acid sequence identity to the amino acid sequence depicted in FIG. 1B, where amino acid 12 is a Cys; i.e., where the β2M comprises a non-naturally-occurring Cys at position 12 as a result of an R12C substitution. In some cases, a β2M polypeptide present in a TMMP comprises the amino acid sequence depicted in FIG. 1B.

### Immunomodulatory Polypeptides

In some cases, an immunomodulatory polypeptide (“MOD”) present in a TMMP is a wild-type (“wt”) MOD. As discussed above, in other cases, a MOD present in a TMMP is a variant of a wt. MOD that has reduced affinity for a co-MOD compared to the affinity of a corresponding wild-type MOD for the co-MOD. Suitable MODs that exhibit reduced affinity for a co-MOD can have from 1 amino acid (aa) to 20 aa differences from a wild-type MOD.

As discussed above, a MOD may comprise a variant of a wt immunomodulatory polypeptide that may exhibit reduced binding to its co-MOD, including e.g., reduced binding to one or more chains or domains of the co-MOD. For example, a variant MOD present in a TMMP may bind its co-MOD with an affinity that it at least 10% less, at least 15% less, at least 20% less, at least 25% less, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, at least 95% less, or more than 95% less, than the affinity of a corresponding wild-type MOD for the co-MOD.

Exemplary pairs of MODs and their co-MODs include, but are not limited to those set out in Table 2, below:

TABLE 2

| Immunomodulatory | |
|---|---|
| Polypeptide (MOD) | Co-MOD |
| 4-1BBL | 4-1BB |
| PD-L1 | PD-1 |
| IL-2 | IL-2 receptor |
| CD80 | CD28 |
| CD86 | CD28 |
| OX40L (CD252) | OX40 (CD134) |
| Fas ligand | Fas |
| ICOS-L | ICOS |
| ICAM | LFA-1 |
| CD30L | CD30 |
| CD40 | CD40L |
| CD83 | CD83L |
| HVEM (CD270) | CD160 |
| JAG1 (CD339) | Notch |
| JAG1 | CD46 |
| CD80 | CTLA4 |
| CD86 | CTLA4 |
| CD70 | CD27 |
| TGFβ | TGFβ receptor |

One or more MODs can be present in a TMMP at any of a variety of positions. For example, in some cases, a MOD (or multiple MODs) can be: i)C-terminal to the MHC class I heavy chain and N-terminal to the Ig Fc polypeptide; in other words, between the MHC class I heavy chain polypeptide and the Ig Fc polypeptide; 2)C-terminal to the Ig Fc polypeptide; or 3)N-terminal to the peptide epitope.

Immunomodulatory polypeptides and variants, including reduced affinity variants, such as PD-L1, CD80, CD86, 4-1BB1L and IL-2 are described in the published literature, e.g., published PCT application WO2020132138A1 and WO2019/051091, the disclosures of which as they pertain to MODs and specific variant MODs of PD-L1, CD80, CD86, 4-1BB1L, IL-2 are expressly incorporated herein by reference, including specifically paragraphs [00260]-[00455] of WO2020132138A1 and paragraphs [00157]-[00352] of WO2019/051091.

Of specific interest are MODs that are variants of the cytokine IL-2. Wild-type IL-2 binds to IL-2 receptor (IL-2R) on the surface of a T cell. Wild-type IL-2 has a strong affinity for IL-2R and will bind to activate most or substantially all CD8+ T cells. For this reason, synthetic forms of wild type IL-2 such as the drug Aldesleukin (trade name Proleukin®) are known to have severe side-effects when administered to humans for the treatment of cancer because the IL-2 indiscriminately activates both target and non-target T cells.

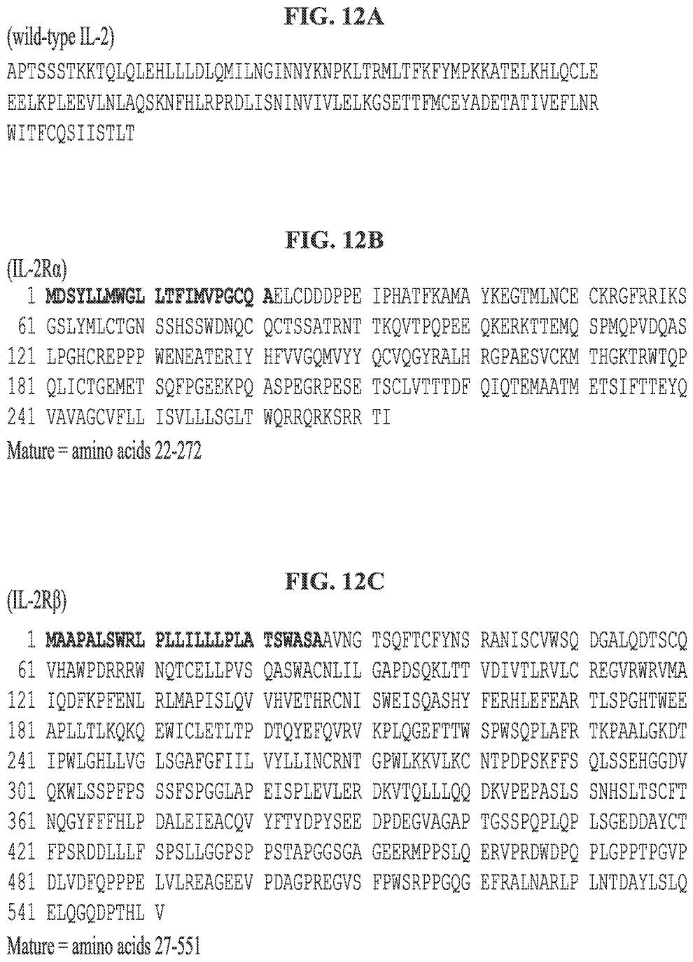

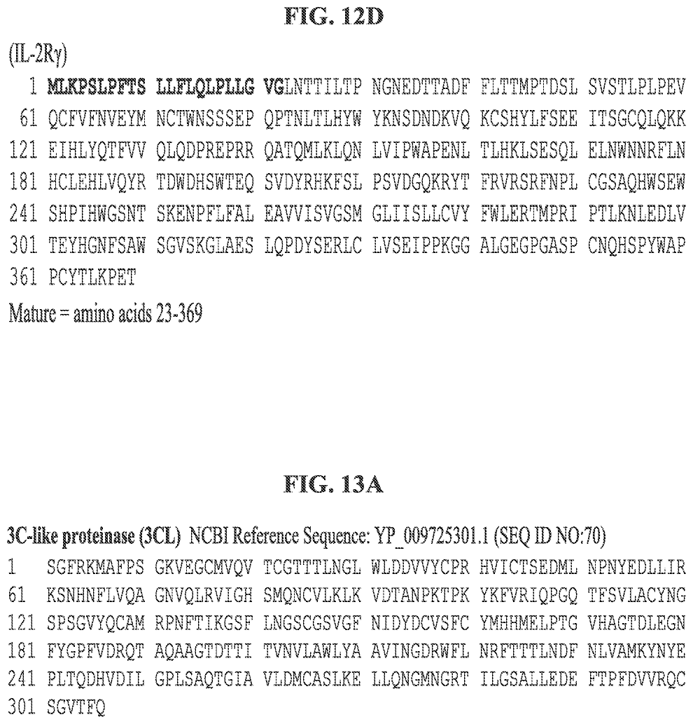

An IL-2 receptor is in some cases a heterotrimeric polypeptide comprising an alpha chain (IL-2Ra; also referred to as CD25), a beta chain (IL-2Rβ; also referred to as CD122; and a gamma chain (IL-2Ry; also referred to as CD132). Amino acid sequences of human IL-2, human IL-2Ra, IL2RP, and IL-2Rγ are known. See, e.g., published PCT applications WO2020132138A1 and WO2019/051091, discussed above. For example, a wild-type IL-2 polypeptide can have the amino acid sequence depicted in FIG. 12A. Amino acid sequences of human IL-2Ra, human IL-2RP, and human IL-2R_{y} are depicted in FIG. 12B, FIG. 12C, and FIG. 12D, respectively. In some cases, the human IL-2Ra, human IL-2RP, and human IL-2R_{y} polypeptides are the mature form (lacking the signal peptide).

In some cases, an IL-2 variant MOD of this disclosure exhibits decreased binding to IL-2Ra, thereby minimizing or substantially reducing the activation of Tregs by the IL-2 variant. Alternatively, or additionally, in some cases, an IL-2 variant MOD of this disclosure exhibits decreased binding to IL-2RP such that the IL-2 variant MOD exhibits an overall reduced affinity for IL-2R. In some cases, an IL-2 variant MOD of this disclosure exhibits both properties, i.e., it exhibits decreased or substantially no binding to IL-2Ra, and also exhibits decreased binding to IL-2RP such that the IL-2 variant polypeptide exhibits an overall reduced affinity for IL-2R. For example, IL-2 variants having substitutions at H16 and F42 have shown decreased binding to IL-2Ra and IL-2RP. See, Quayle et al., Clin Cancer Res; 26(8) Apr. 15, 2020, which discloses that the binding affinity of an IL-2 polypeptide with H16A and F42A substitutions for human IL-2Ra and IL-2RP was decreased 110- and 3-fold, respectively, compared with wild-type IL2 binding, predominantly due to a faster off-rate for each of these interactions. TMMPs comprising such variants, including variants that exhibit decreased binding to IL-2Ra and IL-2Rβ, have shown the ability to preferentially bind to and activate IL-2 receptors on T cells that contain the target TCR that is specific for the peptide epitope on the TMMP, and are thus less likely to deliver IL-2 to non-target T cells, i.e., T cells that do not contain a TCR that specifically binds the peptide epitope on the TMMP. That is, the binding of the IL-2 variant MOD to its costimulatory polypeptide on the T cell is substantially driven by the binding of the MHC-epitope moiety rather than by the binding of the IL-2. In some cases, an IL-2 variant MOD of this disclosure exhibits decreased binding to IL-2Rγ. This decreased binding to IL-2Ry may be in addition to the decreased binding to IL-2Rα and/or IL-2Rβ.

Suitable IL-2 variant MODs thus include a polypeptide that comprises an amino acid sequence having at least 90%, at least 95%, at least 98%, or at least 99% amino acid sequence identity to the wild-type IL-2 amino acid sequence depicted in FIG. 12A; and that have one or more amino acid differences from the wild-type IL-2 amino acid sequence depicted in FIG. 12A that provide for reduced binding to IL-2Ra, IL-2Rβ and/or IL-2Rγ. In some cases, such a variant IL-2 polypeptide of this disclosure exhibits reduced binding affinity to IL-2R, compared to the binding affinity of an IL-2 polypeptide comprising the wild-type IL-2 amino acid sequence depicted in FIG. 12A. For example, in some cases, a variant IL-2 polypeptide binds IL-2R with a binding affinity that is at least 10% less, at least 15% less, at least 20% less, at least 25%, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, at least 95% less, or more than 95% less, than the binding affinity of an IL-2 polypeptide comprising the wild-type IL-2 amino acid sequence depicted in FIG. 12A for an IL-2R (e.g., an IL-2R comprising polypeptides comprising the amino acid sequences depicted in FIG. 12B-12D or comprising the mature forms of the amino acid sequences depicted in FIG. 12B-12D), when assayed under the same conditions.

Mutations that can reduce binding of IL-2 to IL-2Ra include substitutions at one or more of amino acids R38, F42, K43, Y45, E62, P65, E68, V69, and L72. For example, an IL-2 variant can comprise substitutions at one, two, three, four, five or more of the foregoing amino acids. Exemplary substitutions include the following from Table 3:

TABLE 3

| | |
|---|---|
| R38 | Any amino acid other than Arg, e.g., Ala, Asp or Glu |
| F42 | Any amino acid other than Phe, e.g., Ala or Lys, as well |
| | as Met, Pro, Ser, Thr, Trp, Tyr or Val |
| K43 | Any amino acid other than Lys, e.g., Glu |
| Y45 | Any amino acid other than Tyr |
| E62 | Any amino acid other than Glu, e.g., Gln |
| P65 | Any amino acid other than Pro |
| E68 | Any amino acid other than Glu |
| V69 | Any amino acid other than Val |
| L72 | Any amino acid other than Leu |

As noted above, a substitution of F42 with an amino acid other than Phe, e.g., Ala, has been shown to substantially reduce the binding of an IL-2 variant to IL-2Ra. See Quayle et al., Clin Cancer Res 2020; 26:1953-64, which reported that an F42A substitution caused a 110-fold decrease in the binding to IL-2Ra. The binding to IL-2Ra is thus substantially abrogated by this mutation.

Mutations that can reduce binding of IL-2 to IL-2Rβ include substitutions at one or more of amino acids E15, H16, L19, D20, D84, S87, N88, V91, I92. For example, an IL-2 variant can comprise substitutions at one, two, three, four, five or more of the foregoing amino acids. Exemplary substitutions include the following from Table 4:

TABLE 4

| | |
|---|---|
| E15 | Any amino acid other than Glu |
| H16 | Any amino acid other than His, e.g., Ala, Glu, Thr, or Asp, as |
| | well as Gly, Val, Leu, Ile, Arg, Asn, Cys, Glu, Gln, Ile, Lys, |
| | Met, Phe, Pro, Ser, Tyr, Trp or Val |
| L19 | Any amino acid other than Leu |
| D20 | Any amino acid other than Asp, e.g., Asn |
| D84 | Any amino acid other than Asp, e.g., His, Lys, Arg or Lys |
| S87 | Any amino acid other than Ser |
| N88 | Any amino acid other than Asn, e.g., Ser, Ala, Gly, Arg, Thr |
| | or Asp |
| V91 | Any amino acid other than Val, e.g., Glu, Ala or Thr |
| I92 | Any amino acid other than Ile, e.g., Ala |

As noted above, a substitution of H16 with an amino acid other than His, e.g., Ala, has been shown to reduce the binding of an IL-2 variant to IL-2RP3. See Quayle et al., Clin Cancer Res 2020; 26:1953-64, which reported that an H16A substitution caused a 3-fold decrease in the binding to IL-2Rβ.

Some exemplary combinations of mutations that reduce binding of an IL-2 variant polypeptide to IL-2Ra and IL-2Rβ include the following from Table 5:

TABLE 5

| Mutation(s) to | Mutation(s) to | |
|---|---|---|
| decrease binding | decrease binding | |
| to IL-2Rα | to IL-2Rβ | Exemplary combinations |
| R38 with any | E15 with any | E15A with R38A, R38D or R38E |
| amino acid other | amino acid other | |
| than Arg, e.g., Ala, | than Glu | |
| Asp, Glu | | |
| R38 with any | H16, with any | H16A with R38A, R38D or R38E |
| amino acid other | amino acid other | H16T with R38A, R38D or R38E |
| than Arg, e.g., Ala, | than His, e.g., Ala, | H16E with R38A, R38D or R38E |
| Asp, Glu | Glu, Thr, or Asp, | H16D with R38A, R38D or R38E |
| R38 with any | D84, with any | D84H with R38A, R38D or R38E |
| amino acid other | amino acid other | D84K with R38A, R38D or R38E |
| than Arg, e.g., Ala, | than Asp, e.g., His, | D84R with R38A, R38D or R38E |
| Asp, Glu | Lys or Arg | |
| R38 with any | N88, with any | R38A with N88S, N88A, N88G, N88R, N88T, or N88D |
| amino acid other | amino acid other | R38D with N88S, N88A, N88G, N88R, N88T, or N88D |
| than Arg, e.g., Ala, | than Asn, e.g., Ser, | R38E with N88S, N88A, N88G, N88R, N88T, or N88D |
| Asp, Glu | Ala, Gly, Arg, Thr | |
| | or Asp | |
| R38 with any | V91 with any | R38A with V91E, V91A or V91T |
| amino acid other | amino acid other | R38D with V91E, V91A or V91T |
| than Arg, e.g., Ala, | than Val, e.g., Glu, | R38E with V91E, V91A or V91T |
| Asp, Glu | Ala or Thr | |
| R38 with any | I92 with any amino | R38A, I92A |
| amino acid other | acid other than Ile, | R38D, 192A |
| than Arg, e.g., Ala, | e.g., Ala | R38E, I92A |
| Asp, Glu | | |
| F42, with any | E15 with any | E15A, F42A |
| amino acid other | amino acid other | E15A, F42K |
| than Phe, e.g., Ala | than Glu | |
| or Lys,, as well as | | |
| Met, Pro, Ser, Thr, | | |
| Trp, Tyr, and Val | | |
| F42, with any | H16, with any | H16A, F42A; H16T, F42A; |
| amino acid other | amino acid other | H16E, F42A; H16D, F42A |
| than Phe, e.g., Ala | than His, e.g., Ala, | H16A, F42K; H16T, F42K; |
| or Lys, as well as | Glu, Thr, or Asp, | H16E, F42K; H16D, F42K |
| Met, Pro, Ser, Thr, | | |
| Trp, Tyr, and Val | | |
| F42, with any | D84, with any | F42A with D84H, D84K or D84R |
| amino acid other | amino acid other | F42K with D84H, D84K or D84R |
| than Phe, e.g., Ala | than Asp, e.g., His, | |
| or Lys, as well as | Lys or Arg | |
| Met, Pro, Ser, Thr, | | |
| Trp, Tyr, and Val | | |
| F42, with any | N88, with any | F42A with N88S, N88A, N88G, N88R, N88T, or N88D |
| amino acid other | amino acid other | F42K with N88S, N88A, N88G, N88R, N88T, or N88D |
| than Phe, e.g., Ala | than Asn, e.g., Ser, | |
| or Lys, as well as | Ala, Gly, Arg, Thr | |
| Met, Pro, Ser, Thr, | or Asp | |
| Trp, Tyr, and Val | | |
| F42, with any | V91 with any | F42A with V91E, V91A, or V91T |
| amino acid other | amino acid other | F42K with V91E, V91A, or V91T |
| than Phe, e.g., Ala | than Val, e.g., Glu, | |
| or Lys, as well as | Ala or Thr | |
| Met, Pro, Ser, Thr, | | |
| Trp, Tyr, and Val | | |
| F42, with any | I92 with any amino | F42A with I92A |
| amino acid other | acid other than Ile, | F42K with I92A |
| than Phe, e.g., Ala | e.g., Ala | |
| or Lys, as well as | | |
| Met, Pro, Ser, Thr, | | |
| Trp, Tyr, and Val | | |
| K43, with any | E15 with any | E15A, K43E |
| amino acid other | amino acid other | |
| than Lys, e.g., Glu | than Glu | |
| K43, with any | H16, with any | H16A, K43E; H16T, K43E; |
| amino acid other | amino acid other | H16E, K43E; H16D, K43E |
| than Lys, e.g., Glu | than His, e.g., Ala, | |
| | Glu, Thr, or Asp, | |
| K43, with any | D84, with any | K43E with D84H, D84K or D84R |
| amino acid other | amino acid other | |
| than Lys, e.g., Glu | than Asp, e.g., His, | |
| | Lys or Arg | |
| K43, with any | N88, with any | K43E with N88S, N88A, N88G, N88R, N88T, or N88D |
| amino acid other | amino acid other | |
| than Lys, e.g., Glu | than Asn, e.g., Ser, | |
| | Ala, Gly, Arg, Thr | |
| | or Asp | |
| K43, with any | V91 with any | K43E with V91E, V91A, or V91T |
| amino acid other | amino acid other | |
| | than Val, e.g., Glu, | |
| than Lys, e.g., Glu | Ala or Thr | |
| K43, with any | I92 with any amino | K43E, I92A |
| amino acid other | acid other than Ile, | |
| than Lys, e.g., Glu | e.g., Ala | |
| E62, with any | E15 with any | E15A, E62Q |
| amino acid other | amino acid other | |
| than Glu, e.g., Gln | than Glu | |
| E62, with any | H16, with any | H16A, E62Q; H16T, E62Q; |
| amino acid other | amino acid other | H16E, E62Q; H16D, E62Q |
| than Glu, e.g., Gln | than His, e.g., Ala, | |
| | Glu, Thr, or Asp, | |
| E62, with any | D84, with any | E62Q with D84H, D84K or D84R |
| amino acid other | amino acid other | |
| than Glu, e.g., Gln | than Asp, e.g., His, | |
| | Lys or Arg | |
| E62, with any | N88, with any | E62Q with N88S, N88A, N88G, N88R, N88T, or N88D |
| amino acid other | amino acid other | |
| than Glu, e.g., Gln | than Asn, e.g., Ser, | |
| | Ala, Gly, Arg, Thr | |
| | or Asp | |
| E62, with any | V91 with any | E62Q with V91E, V91A, or V91T |
| amino acid other | amino acid other | |
| than Glu, e.g., Gln | than Val, e.g., Glu, | |
| | Ala or Thr | |
| E62, with any | I92 with any amino | E62Q, I92A |
| amino acid other | acid other than Ile, | |
| than Glu, e.g., Gln | e.g., Ala | |
| F42, with any | E15 with any | E15A, F42A with D84H, D84K or D84R |
| amino acid other | amino acid other | E15A, F42K with D84H, D84K or D84R |
| than Phe, e.g., Ala | than Glu | |
| or Lys, as well as | D84, with any | |
| Met, Pro, Ser, Thr, | amino acid other | |
| Trp, Tyr, and Val | than Asp, e.g., His, | |
| | Lys and Arg | |
| F42, with any | E15 with any | E15A, F42A with N88S, N88A, N88G, N88R, N88T, or |
| amino acid other | amino acid other | N88D |
| than Phe, e.g., Ala | than Glu | E15A, F42K with N88S, N88A, N88G, N88R, N88T, or |
| or Lys, as well as | N88, with any | N88D |
| Met, Pro, Ser, Thr, | amino acid other | |
| Trp, Tyr, and Val | than Asn, e.g., Ser, | |
| | Ala, Gly, Arg, Thr, | |
| | and Asp | |
| F42, with any | E15 with any | E15A, F42A with V91E, V91A, or V91T |
| amino acid other | amino acid other | E15A, F42K with V91E, V91A, or V91T |
| than Phe, e.g., Ala | than Glu | |
| or Lys, as well as | V91 with any | |
| Met, Pro, Ser, Thr, | amino acid other | |
| Trp, Tyr, and Val | than Val, e.g., Glu, | |
| | Ala or Thr | |
| F42, with any | E15 with any | E15A, F42A with 192A |
| amino acid other | amino acid other | E15A, F42K with I92A |
| than Phe, e.g., Ala | than Glu | |
| or Lys, as well as | I92 with any amino | |
| Met, Pro, Ser, Thr, | acid other than Ile, | |
| Trp, Tyr, and Val | e.g., Ala | |
| F42, with any | H16, with any | H16A, F42A with D84H, D84K or D84R |
| amino acid other | amino acid other | H16A, F42K with D84H, D84K or D84R |
| than Phe, e.g., Ala | than His, e.g., Ala, | H16T, F42A with D84H, D84K or D84R |
| or Lys, as well as | Glu, Thr, or Asp | H16T, F42K with D84H, D84K or D84R |
| Met, Pro, Ser, Thr, | D84, with any | H16E, F42A with D84H, D84K or D84R |
| Trp, Tyr, and Val | amino acid other | H16E, F42K with D84H, D84K or D84R |
| | than Asp, e.g., His, | H16D, F42A with D84H, D84K or D84R |
| | Lys and Arg | H16D, F42K with D84H, D84K or D84R |
| F42, with any | H16, with any | H16A, F42A with N88S, N88A, N88G, N88R, N88T, or |
| amino acid other | amino acid other | N88D |
| than Phe, e.g., Ala | than His, e.g., Ala, | H16A, F42K with N88S, N88A, N88G, N88R, N88T, or |
| or Lys, as well as | Glu, Thr, or Asp | N88D |
| Met, Pro, Ser, Thr, | N88, with any | H16T, F42A with N88S, N88A, N88G, N88R, N88T, or |
| Trp, Tyr, and Val | amino acid other | N88D |
| | than Asn, e.g., Ser, | H16T, F42K with N88S, N88A, N88G, N88R, N88T, or |
| | Ala, Gly, Arg, Thr, | N88D |
| | and Asp | H16E, F42A with N88S, N88A, N88G, N88R, N88T, or |
| | | N88D |
| | | H16E, F42K with N88S, N88A, N88G, N88R, N88T, or |
| | | N88D |
| | | H16D, F42A with N88S, N88A, N88G, N88R, N88T, or |
| | | N88D |
| | | H16D, F42K with N88S, N88A, N88G, N88R, N88T, or |
| | | N88D |
| F42, with any | H16, with any | H16A, F42A with V91E, V91A, or V91T |
| amino acid other | amino acid other | H16A, F42K with V91E, V91A, or V91T |
| than Phe, e.g., Ala | than His, e.g., Ala, | H16T, F42A with V91E, V91A, or V91T |
| or Lys, as well as | Glu, Thr, or Asp | H16T, F42K with V91E, V91A, or V91T |
| Met, Pro, Ser, Thr, | V91 with any | H16E, F42A with V91E, V91A, or V91T |
| Trp, Tyr, and Val | amino acid other | H16E, F42K with V91E, V91A, or V91T |
| | than Val, e.g., Glu, | H16D, F42A with V91E, V91A, or V91T |
| | Ala or Thr | H16D, F42K with V91E, V91A, or V91T |
| F42, with any | H16, with any | H16A, F42A with I92A |
| amino acid other | amino acid other | H16A, F42K with I92A |
| than Phe, e.g., Ala | than His, e.g., Ala, | H16T, F42A with I92A |
| or Lys, as well as | Glu, Thr, or Asp | H16T, F42K with I92A |
| Met, Pro, Ser, Thr, | 192 with any amino | H16E, F42A with I92A |
| Trp, Tyr, and Val | acid other than Ile, | H16E, F42K with I92A |
| | e.g., Ala | H16D, F42A with I92A |
| | | H16D, F42K with I92A |

Exemplary IL-2 variant polypeptide amino acid sequences comprise an amino acid sequence having at least about least 85%, at least 90%, at least 95%, at least 98%, at least 99%, or 100%, amino acid sequence identity to any one of the following IL-2 variant amino acid sequences (I)-(XVIII):
(1) APTSSSTKKT QLQLEHLLLD LQMILNGINN YKNPKLTX₁ML TFKFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO: 1225), wherein X₁ is any amino acid other than Arg, e.g., Ala, Asp or Glu;
(II) APTSSSTKKT QLQLEHLLLD LQMILNGINN YKNPKLTRML TX₁KFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:1226), wherein X₁ is any amino acid other than Phe, e.g., Ala or Lys, and in some cases, X₁ instead may be Gly, Val, Leu, Ile, Arg, Asn, Cys, Glu, Gln, Ile, Lys, Met, Phe, Pro, Ser, Tyr, Trp or Val;
(III) APTSSSTKKT QLQLEHLLLD LQMILNGINN YKNPKLTRML TFX₁FYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:1227), wherein X₁ is any amino acid other than Lys, e.g., Glu;
(IV) APTSSSTKKT QLQLEHLLLD LQMILNGINN YKNPKLTRML TFKFX₁MPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:1228), wherein X₁ is any amino acid other than Tyr;
(V) APTSSSTKKT QLQLEHLLLD LQMILNGINN YKNPKLTRML TFKFYMPKKA TELKHLQCLE EX₁LKPLEEVL NLAQSKNFHL RPRDLISNIN VIVLELKGSE TTFMCEYADE TATIVEFLNR WITFCQSIIS TLT (SEQ ID NO:1229), wherein X₁ is any amino acid other than Glu, e.g., Gln;
(VI) APTSSSTKKT QLQLEHLLLD LQMILNGINN YKNPKLTRML TFKFYMPKKA TELKHLQCLE EELKX₁LEEVL NLAQSKNFHL RPRDLISNIN VIVLELKGSE TTFMCEYADE TATIVEFLNR WITFCQSIIS TLT (SEQ ID NO:1230), wherein X₁ is any amino acid other than Pro;
(VII) APTSSSTKKT QLQLEHLLLD LQMILNGINN YKNPKLTRML TFKFYMPKKA TELKHLQCLE EELPKLEX₁VL NLAQSKNFHL RPRDLISNIN VIVLELKGSE TTFMCEYADE TATIVEFLNR WITFCQSIIS TLT (SEQ ID NO:1231), wherein X₁ is any amino acid other than Glu;
(VIII) APTSSSTKKT QLQLEHLLLD LQMILNGINN YKNPKLTRML TFKFYMPKKA TELKHLQCLE EELPKLEEX₁L NLAQSKNFHL RPRDLISNIN VIVLELKGSE TTFMCEYADE TATIVEFLNR WITFCQSIIS TLT (SEQ ID NO:1232), wherein X₁ is any amino acid other than Val;
(IX) APTSSSTKKT QLQLEHLLLD LQMILNGINN YKNPKLTRML TFKFYMPKKA TELKHLQCLE EELPKLEEVL NX₁AQSKNFHL RPRDLISNIN VIVLELKGSE TTFMCEYADE TATIVEFLNR WITFCQSIIS TLT (SEQ ID NO:1233), wherein X₁ is any amino acid other than Leu;
(X) APTSSSTKKT QLQLX₁HLLLD LQMILNGINN YKNPKLTRML TFKFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:1234), wherein X₁ is any amino acid other than Glu, e.g., Ala;
(XI) APTSSSTKKT QLQLEX₁LLLD LQMILNGINN YKNPKLTRML TFKFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:1235), wherein X₁ is any amino acid other than His, e.g., Ala, Glu, Thr, or Asp, and in some cases, X₁ is Gly, Val, Leu, Ile, Arg, Asn, Cys, Glu, Gln, Ile, Lys, Met, Phe, Pro, Ser, Tyr, Trp or Val;
(XII) APTSSSTKKT QLQLEHLLX₁D LQMILNGINN YKNPKLTRML TFKFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:1236), wherein X₁ is any amino acid other than Leu;
(XIII)APTSSSTKKT QLQLEHLLLX₁LQMILNGINN YKNPKLTRML TFKFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:1237), wherein X₁ is any amino acid other than Asp, e.g., Asn;
(XIV) APTSSSTKKT QLQLEHLLLD LQMILNGINN YKNPKLTRML TFKFYMPKKA TELKHLQCLE EELKPLEEVL NLAQSKNFHL RPRX₁LISNIN VIVLELKGSE TTFMCEYADE TATIVEFLNR WITFCQSIIS TLT (SEQ ID NO:1238), wherein X₁ is any amino acid other than Asp, e.g., His, Lys or Arg;
(XV) APTSSSTKKT QLQLEHLLLD LQMILNGINN YKNPKLTRML TFKFYMPKKA TELKHLQCLE EELKPLEEVL NLAQSKNFHL RPRDLI X₁NIN VIVLELKGSE TTFMCEYADE TATIVEFLNR WITFCQSIIS TLT (SEQ ID NO:1239), wherein X₁ is any amino acid other than Ser;
(XVI) APTSSSTKKT QLQLEHLLLD LQMILNGINN YKNPKLTRML TFKFYMPKKA TELKHLQCLE EELKPLEEVL NLAQSKNFHL RPRDLISX₁IN VIVLELKGSE TTFMCEYADE TATIVEFLNR WITFCQSIIS TLT (SEQ ID NO:1240), wherein X₁ is any amino acid other than Asn, e.g., Ser, Ala, Gly, Arg, Thr or Asp;
(XVII) APTSSSTKKT QLQLEHLLLD LQMILNGINN YKNPKLTRML TFKFYMPKKA TELKHLQCLE EELKPLEEVL NLAQSKNFHL RPRDLISNIN X₁IVLELKGSE TTFMCEYADE TATIVEFLNR WITFCQSIIS TLT (SEQ ID NO:1241), wherein X₁ is any amino acid other than Val, e.g., Glu, Ala or Thr; and
(XVIII) APTSSSTKKT QLQLEHLLLD LQMILNGINN YKNPKLTRML TFKFYMPKKA TELKHLQCLE EELKPLEEVL NLAQSKNFHL RPRDLISNIN VX₁VLELKGSE TTFMCEYADE TATIVEFLNR WITFCQSIIS TLT (SEQ ID NO:1242), wherein X₁ is any amino acid other than Ile, e.g., Ala.

In any of the foregoing IL-2 variant amino acid sequences (I)-(XVIII), the amino acid sequence may comprise one or more additional mutations that reduce the binding of the IL-2 variant to IL-2Rα and/or IL-2Rβ (as compared to the binding affinity of wild-type IL-2 to IL-2Rα and/or IL-2RP).

The determination of binding affinity may be carried out by known methods.

In some cases, a suitable variant IL-2 polypeptide comprises an amino acid sequence having at least 90%, at least 95%, at least 98%, at least 99%, or 100% amino acid sequence identity to the amino acid sequence: APTSSSTKKT QLQLEX₁LLLD LQMILNGINN YKNPKLTRML TX₂KFYMPKKA TELKHLQCLEEELKPLEEVL NLAQSKNFHL RPRDLISNIN VIVLELKGSE TTFMCEYADE TATIVEFLNRWITFCQSIIS TLT (SEQ ID NO:1243), i.e., the variant IL-2 polypeptide has the amino acid sequence of wild-type IL-2 but with H16 and F42 substitutions (X₁ and X₂, shown in bold). As noted above, the histidine at position 16 can be substituted with, e.g., Ala, Glu, Thr, or Asp, but any of the following substitutions also may be employed: Gly, Val, Leu, Ile, Arg, Asn, Cys, Glu, Gln, Ile, Lys, Met, Phe, Pro, Ser, Tyr, or Trp. The phenylalanine at position 42 can be substituted with, e.g., Ala or Lys, but any of the following substitutions also may be employed: Met, Pro, Ser, Thr, Trp, Tyr, or Val.

In some cases, a variant IL-2 polypeptide present in a TMMP comprises the amino acid sequence: APTSSSTKKT QLQLEALLLD LQMILNGINN YKNPKLTRML TAKFYMPKKA TELKHLQCLEEELKPLEEVL NLAQSKNFHL RPRDLISNIN VIVLELKGSE TTFMCEYADE TATIVEFLNRWITFCQSIIS TLT (SEQ ID NO:878).

In some cases, a variant IL-2 polypeptide present in a TMMP comprises the amino acid sequence: APTSSSTKKT QLQLETLLLD LQMILNGINN YKNPKLTRML TAKFYMPKKA TELKHLQCLEEELKPLEEVL NLAQSKNFHL RPRDLISNIN VIVLELKGSE TTFMCEYADE TATIVEFLNRWITFCQSIIS TLT (SEQ ID NO:925). In some cases, a TMMP comprises two copies of such a variant IL-2 polypeptide.

In some cases, a variant IL-2 polypeptide present in a TMMP comprises the amino acid sequence: APTSSSTKKT QLQLEELLLD LQMILNGINN YKNPKLTRML TAKFYMPKKA TELKHLQCLEEELKPLEEVL NLAQSKNFHL RPRDLISNIN VIVLELKGSE TTFMCEYADE TATIVEFLNRWITFCQSIIS TLT (SEQ ID NO:1244). In some cases, a TMMP comprises two copies of such a variant IL-2 polypeptide.

In some cases, a variant IL-2 polypeptide present in a TMMP comprises the amino acid sequence: APTSSSTKKT QLQLEDLLLD LQMILNGINN YKNPKLTRML TAKFYMPKKA TELKHLQCLEEELKPLEEVL NLAQSKNFHL RPRDLISNIN VIVLELKGSE TTFMCEYADE TATIVEFLNRWITFCQSIIS TLT (SEQ ID NO:1245). In some cases, a TMMP comprises two copies of such a variant IL-2 polypeptide.

As further examples of MODS, in some cases, a MOD present in a TMMP is a 4-1BBL polypeptide. In some cases, a 4-1BBL polypeptide of a TMMP comprises an amino acid sequence having at least 75%, at least 80%, at least 85%, at least 90%, at least 95%, at least 98%, at least 99%, or 100%, amino acid sequence identity to the following 4-1BBL amino acid sequence: DPAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPA (SEQ ID NO:926).

In some cases, a MOD present in a TMMP is a CD80 polypeptide. In some cases, a CD80 polypeptide of a TMMP comprises an amino acid sequence having at least 75%, at least 80%, at least 85%, at least 90%, at least 95%, at least 98%, at least 99%, or 100%, amino acid sequence identity to following CD80 amino acid sequence: VIHVTK EVKEVATLSC GHNVSVEELA QTRIYWQKEK KMVLTMMSGD MNIWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO: (SEQ ID NO:927).

In some cases, a MOD present in a TMMP is a CD86 polypeptide. In some cases, a CD86 polypeptide of a TMMP comprises an amino acid sequence having at least 75%, at least 80%, at least 85%, at least 90%, at least 95%, at least 98%, at least 99%, or 100%, amino acid sequence identity to the following CD86 amino acid sequence:

(SEQ ID NO: 928)
APLKIQAYFNETADLPCQFANSQNQSLSELVVFWQDQENLVLNEVYLGKE
KFDSVHSKYMNRTSFDSDSWTLRLHNLQIKDKGLYQCIIHHKKPTGMIRI
HQMNSELSVLANFSQPEIVPISNITENVYINLTCSSIHGYPEPKKMSVLL
RTKNSTIEYDGIMQKSQDNVTELYDVSISLSVSFPDVTSNMTIFCILETD
KTRLLSSPFSIELEDPQPPPDHIP.

### Anti-CD28 Polypeptides

In some cases, an immunomodulatory polypeptide present in a TMMP is an anti-CD28 antibody (e.g., an anti-CD28 scFv; an anti-CD28 nanobody; an anti-CD28 diabody; and the like). The ability of anti-CD28 antibodies to act as a superagonist, agonist, or antagonist of CD28 activity has been described. See e.g., Poirier et al., (2012) Amer. J. of Transplantation, “CD28-Specific Immunomodulating Antibodies: What Can Be Learned From Experimental Models?” 12:1682-1690. Of particular interest are anti-CD28 antibodies that act as an agonist or superagonist.

Thus, in some cases, a TMMP comprises an anti-CD28 antibody as at least one of the one or more immunomodulatory polypeptides. In some cases, a TMMP comprises, as the one or more immunomodulatory polypeptides: a) an anti-CD28 antibody; and b) one or more IL-2 polypeptides. In some cases, a TMMP comprises, as the one or more immunomodulatory polypeptides: a) an anti-CD28 antibody; and b) one or more IL-2 polypeptides, where the one or more IL-2 polypeptide are variant IL-2 polypeptides, as described above (e.g., an IL-2 polypeptide comprising H16A and F42A substitutions; an IL-2 polypeptide comprising H16T and F42A substitutions; an IL-2 polypeptide comprising H16T and F42T substitutions; and IL-2 polypeptide comprising H16A and F42T substitutions; and the like). In some cases, a TMMP comprises, as the one or more immunomodulatory polypeptides: a) an anti-CD28 antibody; and b) two copies of an IL-2 polypeptide comprising H16A and F42A substitutions. Examples of anti-CD28 VL and VH polypeptides are provided in below. In some cases, a TMMP comprises, as the one or more immunomodulatory polypeptides: a) an anti-CD28 antibody; and b) two copies of an IL-2 polypeptide comprising H16T and F42A substitutions. In some cases, a TMMP comprises, as the one or more immunomodulatory polypeptides: a) an anti-CD28 antibody; and b) one, two, or three copies of a 4-1BBL polypeptide. In some cases, a TMMP comprises, as the one or more immunomodulatory polypeptides: a) an anti-CD28 antibody; and b) three copies of a 4-1BBL polypeptide comprising a wild-type amino acid sequence. In some cases, a TMMP comprises, as the one or more immunomodulatory polypeptides: a) an anti-CD28 antibody; and b) three copies of a variant 4-1BBL polypeptide. The two different immunomodulatory polypeptides can be at any of a number of different positions in the first or the second polypeptide of the heterodimer of the TMMP. For example, in some cases, the anti-CD28 antibody is at the C-terminus of the first polypeptide; and the second immunomodulatory polypeptide (e.g., an IL-2 polypeptide (e.g., a variant IL-2 polypeptide); a 4-1BBL polypeptide; etc.) is at the N-terminus of the second polypeptide. As another example, in some cases, the anti-CD28 antibody is at the N-terminus of the second polypeptide; and the second immunomodulatory polypeptide (e.g., an IL-2 polypeptide (e.g., a variant IL-2 polypeptide); a 4-1BBL polypeptide; etc.) is at the C-terminus of the second polypeptide.

In some cases, an anti-CD28 antibody suitable for inclusion as an immunomodulatory polypeptide in a TMMP comprises: a) VL CDR1, VL CDR2, and VL CDR3 present in a light chain variable region (VL) comprising the following amino acid sequence: QWYQQKPGQPPKLLIFAASNVESGVPARFSGSGSGTNFSLNIHPVDEDDVAMYFCQQSRKVPYT FGGGTKLEIKR (SEQ ID NO:929); and b) VH CDR1, CDR2, and CDR3 present in a heavy chain variable region (VH) comprising the following amino acid sequence: QVKLQQSGPGLVTPSQSLSITCTVSGFSLSDYGVHWVRQSPGQGLEWLGVIWAGGGTNYNSAL MSRKSISKDNSKSQVFLKMNSLQADDTAVYYCARDKGYSYYYSMDYWGQGTTVTVSS (SEQ ID NO:930). In some cases, the V_{H} and V_{L} CDRs are as defined by Kabat (see, e.g., Table 1, above; and Kabat 1991). In some cases, the V_{H} and V_{L} CDRs are as defined by Chothia (see, e.g., Table 1, above; and Chothia 1987). In some cases, the VH CDRs are: DYGVH (SEQ ID NO:931) (VH CDR1); VIWAGGGTNYNSALMS (SEQ ID NO:932) (VH CDR2); and DKGYSYYYSMDY (SEQ ID NO:933) (VH CDR3).

In some cases, an anti-CD28 antibody suitable for inclusion as an immunomodulatory polypeptide in a TMMP comprises: a) a VL region comprising an amino acid sequence having at least 90%, at least 95%, at least 98%, at least 99%, or 100%, amino acid sequence identity to the following amino acid sequence: QWYQQKPGQPPKLLIFAASNVESGVPARFSGSGSGTNFSLNIHPVDEDDVAMYFCQQSRKVPYT FGGGTKLEIKR (SEQ ID NO:929); and b) a VH region comprising an amino acid sequence having at least 90%, at least 95%, at least 98%, at least 99%, or 100%, amino acid sequence identity to the following amino acid sequence:

(SEQ ID NO: 930)
QVKLQQSGPGLVTPSQSLSITCTVSGFSLSDYGVHWVRQSPGQGLEWLGV
IWAGGGTNYNSALMSRKSISKDNSKSQVFLKMNSLQADDTAVYYCARDKG
YSYYYSMDYWGQGTTVTVSS.

In some cases, an anti-CD28 antibody suitable for inclusion as an immunomodulatory polypeptide in a TMMP is a scFv comprising, in order from N-terminus to C-terminus: a) a VL region comprising an amino acid sequence having at least 90%, at least 95%, at least 98%, at least 99%, or 100%, amino acid sequence identity to the following amino acid sequence: QWYQQKPGQPPKLLIFAASNVESGVPARFSGSGSGTNFSLNIHPVDEDDVAMYFCQQSRKVPYT FGGGTKLEIKR (SEQ ID NO:929); b) a peptide linker; and c) a VH region comprising an amino acid sequence having at least 90%, at least 95%, at least 98%, at least 99%, or 100%, amino acid sequence identity to the following amino acid sequence: QVKLQQSGPGLVTPSQSLSITCTVSGFSLSDYGVHWVRQSPGQGLEWLGVIWAGGGTNYNSAL MSRKSISKDNSKSQVFLKMNSLQADDTAVYYCARDKGYSYYYSMDYWGQGTTVTVSS (SEQ ID NO:930). In some cases, the peptide linker comprises the amino acid sequence (GGGGS)n (SEQ ID NO:934), where n is an integer from 1 to 10 (e.g., where n is 1, 2, 3, 4, 5, 6, 7, 8, 9, or 10). In some cases, the peptide linker comprises the amino acid sequence GGGGSGGGGSGGGGS (SEQ ID NO:875) and has a length of 15 amino acids.

In some cases, an anti-CD28 antibody suitable for inclusion as an immunomodulatory polypeptide in a TMMP is a scFv comprising, in order from N-terminus to C-terminus: a) a VH region comprising an amino acid sequence having at least 90%, at least 95%, at least 98%, at least 99%, or 100%, amino acid sequence identity to the following amino acid sequence: QVKLQQSGPGLVTPSQSLSITCTVSGFSLSDYGVHWVRQSPGQGLEWLGVIWAGGGTNYNSAL MSRKSISKDNSKSQVFLKMNSLQADDTAVYYCARDKGYSYYYSMDYWGQGTTVTVSS (SEQ ID NO:930); b) a peptide linker; and c) a VL region comprising an amino acid sequence having at least 90%, at least 95%, at least 98%, at least 99%, or 100%, amino acid sequence identity to the following amino acid sequence: QWYQQKPGQPPKLLIFAASNVESGVPARFSGSGSGTNFSLNIHPVDEDDVAMYFCQQSRKVPYT FGGGTKLEIKR (SEQ ID NO:929). In some cases, the peptide linker comprises the amino acid sequence (GGGGS)n (SEQ ID NO:934), where n is an integer from 1 to 10 (e.g., where n is 1, 2, 3, 4, 5, 6, 7, 8, 9, or 10). In some cases, the peptide linker comprises the amino acid sequence GGGGSGGGGSGGGGS (SEQ ID NO:875) and has a length of 15 amino acids.

### Immunomodulatory Polypeptide and TTP Positions

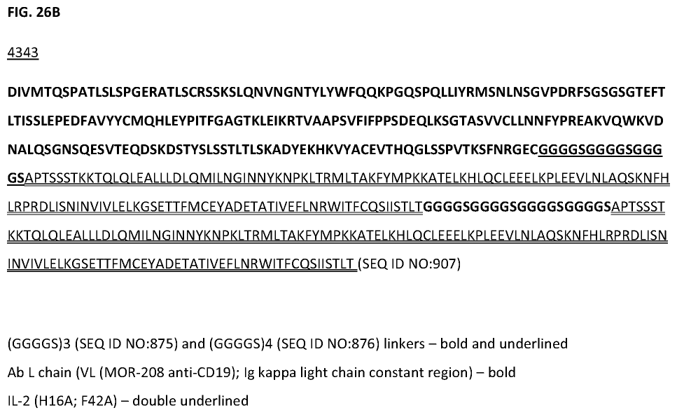

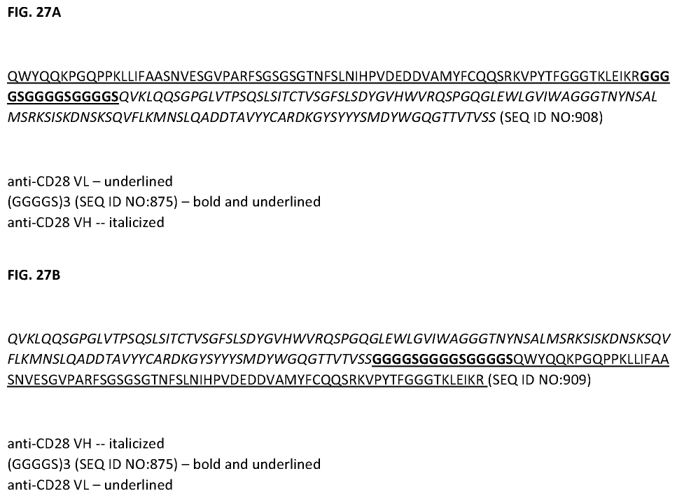

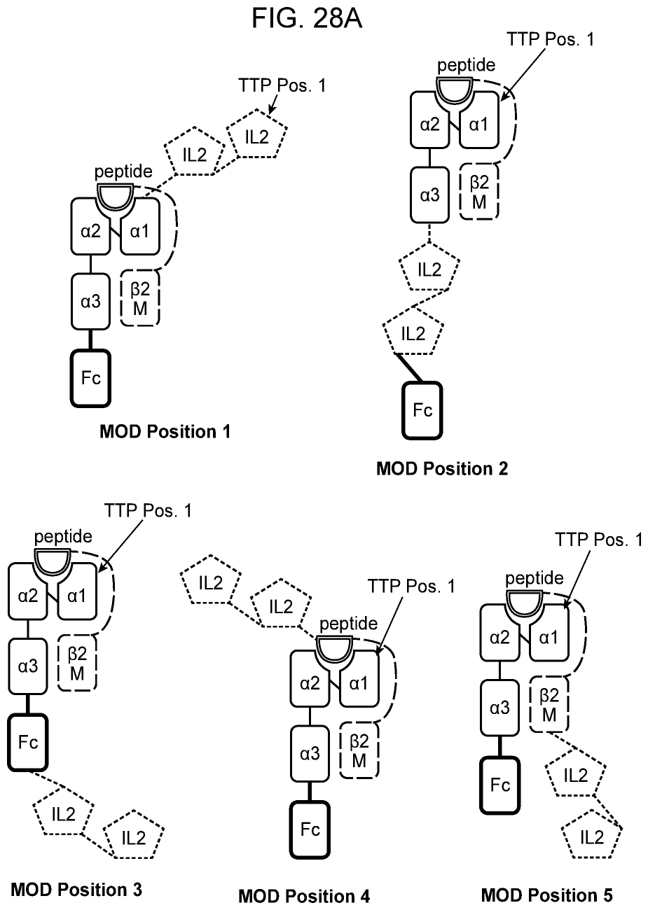

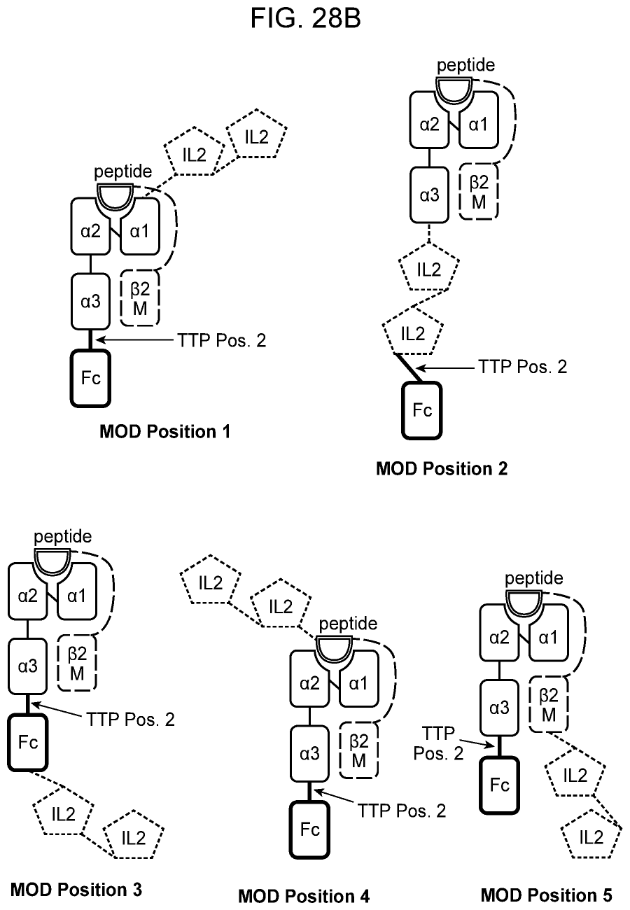

An immunomodulatory polypeptide (i.e., one or more immunomodulatory polypeptides) can be present in a TMMP at any of a variety of positions. For example, an immunomodulatory polypeptide can be: 1)N-terminal to the MHC class I heavy chain (position 1); 2)C-terminal to the MHC class I heavy chain and N-terminal to the Ig Fc polypeptide; in other words, between the MHC class I heavy chain and the Ig Fc polypeptide (position 2); 3)C-terminal to the Ig Fc polypeptide (position 3); 4)N-terminal to the peptide epitope (position 4); or 5)C-terminal to the β2M polypeptide (position 5). Similarly, a TTP can be present in a TMMP at any of a variety of positions. Non-limiting examples of the various MOD positions and the various TTP positions are illustrated in FIGS. 28A-28C.

Of the embodiments illustrated in FIGS. 28A-28C, configurations where the MOD(s) is/are in Position 1 or Position 3, and where the TTP is in Position 1 or Position 3, e.g., (MOD Position 1; TTP Position 3) and (MOD Position 3; TTP Position 1) may be more commonly employed. As also noted above, in any of the above embodiments, one or more of the individual components (e.g., peptide epitope; β2M polypeptide; TTP; etc.) of the first polypeptide in each of the above TMMP combinations are linked together by one or more linkers (e.g., peptide linkers, as described elsewhere herein). In any of the above embodiments, one or more of the individual components (e.g., MHC class I heavy chain; immunomodulatory polypeptide; Ig Fc polypeptide; etc.) of the second polypeptide in each of the above TMMP combinations are linked together by one or more linkers (e.g., peptide linkers, as described elsewhere herein).

### Scaffold Polypeptides

A TMMP can comprise an Fc polypeptide or can comprise another suitable scaffold polypeptide.

Suitable scaffold polypeptides include antibody-based scaffold polypeptides and non-antibody-based scaffolds. Non-antibody-based scaffolds include, e.g., albumin, an XTEN (extended recombinant) polypeptide, transferrin, an Fc receptor polypeptide, an elastin-like polypeptide (see, e.g., Hassouneh et al. (2012) Methods Enzymol. 502:215; e.g., a polypeptide comprising a pentapeptide repeat unit of (Val-Pro-Gly-X-Gly; SEQ ID NO:935), where X is any amino acid other than proline), an albumin-binding polypeptide, a silk-like polypeptide (see, e.g., Valluzzi et al. (2002) Philos Trans R Soc Lond B Biol Sci. 357:165), a silk-elastin-like polypeptide (SELP; see, e.g., Megeed et al. (2002) Adv Drug Deliv Rev. 54:1075), and the like. Suitable XTEN polypeptides include, e.g., those disclosed in WO 2009/023270, WO 2010/091122, WO 2007/103515, US 2010/0189682, and US 2009/0092582; see also Schellenberger et al. (2009) Nat Biotechnol. 27:1186). Suitable albumin polypeptides include, e.g., human serum albumin.

Suitable scaffold polypeptides will in some cases be a half-life extending polypeptides. Thus, in some cases, a suitable scaffold polypeptide increases the in vivo half-life (e.g., the serum half-life) of the TMMP, compared to a control TMMP lacking the scaffold polypeptide. For example, in some cases, a scaffold polypeptide increases the in vivo half-life (e.g., the serum half-life) of the TMMP, compared to a control TMMP lacking the scaffold polypeptide, by at least about 10%, at least about 15%, at least about 20%, at least about 25%, at least about 50%, at least about 2-fold, at least about 2.5-fold, at least about 5-fold, at least about 10-fold, at least about 25-fold, at least about 50-fold, at least about 100-fold, or more than 100-fold. As an example, in some cases, an Fc polypeptide increases the in vivo half-life (e.g., the serum half-life) of the TMMP, compared to a control TMMP lacking the Fc polypeptide, by at least about 10%, at least about 15%, at least about 20%, at least about 25%, at least about 50%, at least about 2-fold, at least about 2.5-fold, at least about 5-fold, at least about 10-fold, at least about 25-fold, at least about 50-fold, at least about 100-fold, or more than 100-fold.

### Fc Polypeptides

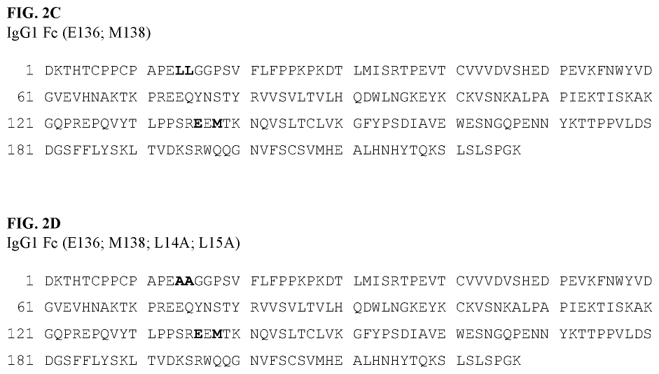

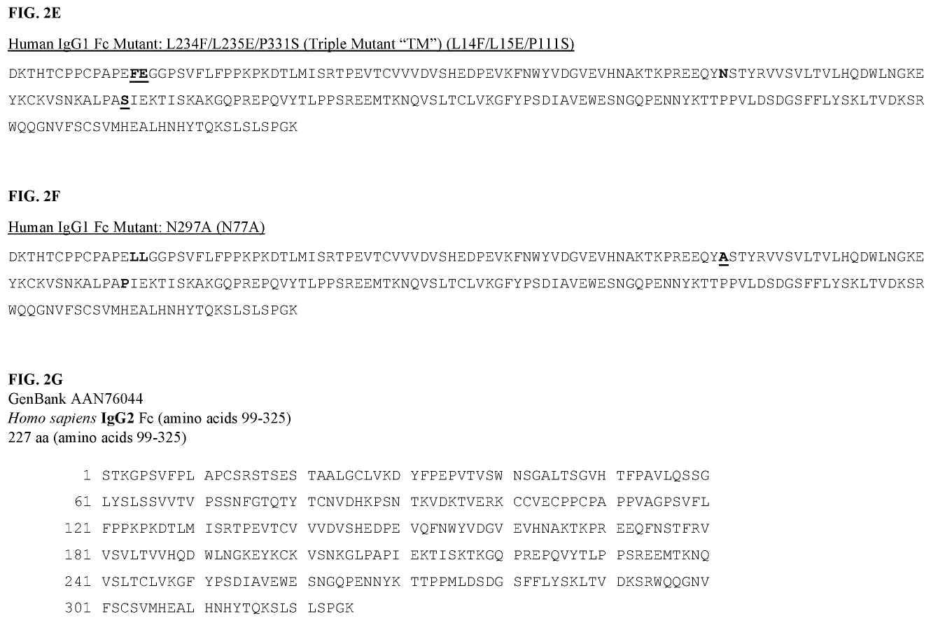

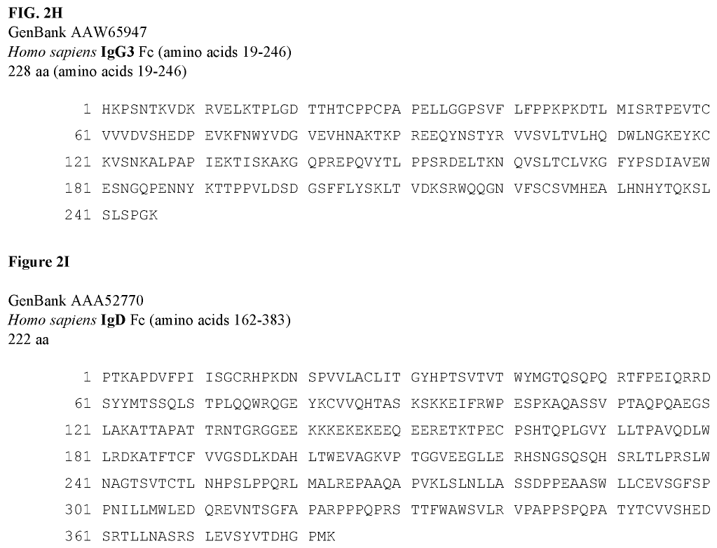

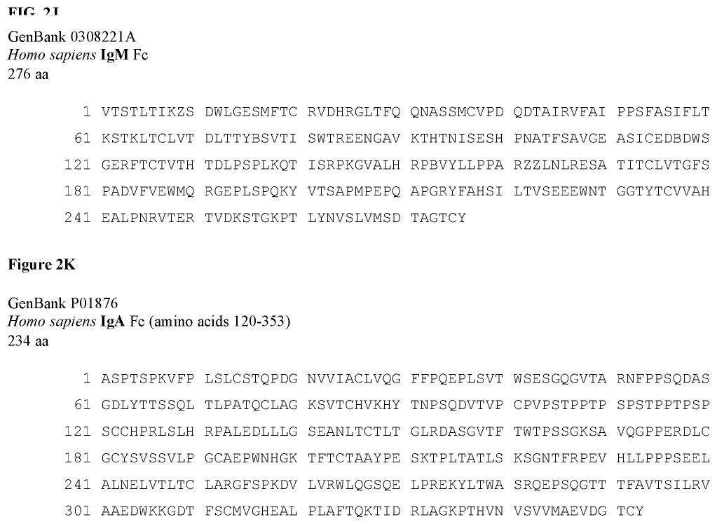

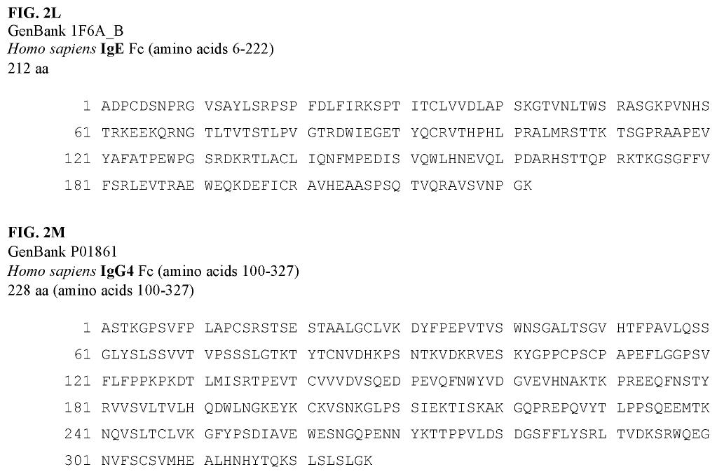

In some cases, a TMMP comprises an Ig Fc polypeptide. An Ig Fc polypeptide is also referred to herein as an “Fc polypeptide.” The Ig Fc polypeptide of a TMMP can be a human IgG1 Fc, a human IgG2 Fc, a human IgG3 Fc, a human IgG4 Fc, etc., or a variant of a wild-type Ig Fc polypeptide. Variants include naturally occurring variants, non-naturally occurring variants, and combinations thereof. For example, the Ig Fc can be a variant of a Fc polypeptide such as a human IgG1 Fc, which variant has a substantially reduced ability to effect complement-dependent cytotoxicity (CDC) or antibody-dependent cell cytotoxicity (ADCC). See, e.g., the variant human IgG1 Fc polypeptides of FIG. 2B and FIG. 2D).

In some cases, the Fc polypeptide present in a TMMP comprises an amino acid sequence having at least 70%, at least 75%, at least 80%, at least 85%, at least 90%, at least 95%, at least 98%, at least 99%, or 100%, amino acid sequence identity to the Fc amino acid sequence depicted in any one of FIG. 2A-2M.

In some cases, the Fc polypeptide present in a TMMP is an IgG1 Fc polypeptide, or a variant of an IgG1 Fc polypeptide. For example, in some cases, the Fc polypeptide present in a TMMP comprises an amino acid sequence having at least 70%, at least 75%, at least 80%, at least 85%, at least 90%, at least 95%, at least 98%, at least 99%, or 100%, amino acid sequence identity to the human IgG1 Fc polypeptide depicted in FIG. 2A. As another example, in some cases, the Fc polypeptide present in a TMMP comprises an amino acid sequence having at least 70%, at least 75%, at least 80%, at least 85%, at least 90%, at least 95%, at least 98%, at least 99%, or 100%, amino acid sequence identity to the Fc polypeptide depicted in FIG. 2B, where the Ig Fc polypeptide comprises an Ala at position 14 and an Ala at position 15. In any of the above embodiments, the Ig Fc polypeptide can have an N77 substitution, i.e., the Ig Fc polypeptide can have an amino acid other than Asn at position 77, where in some cases, the Ig Fc polypeptide has an Ala at position 77. In some cases, an Fc polypeptide present in a TMMP comprises the amino acid sequence depicted in FIG. 2A. In some cases, an Fc polypeptide present in a TMMP comprises the amino acid sequence depicted in FIG. 2B.

In some cases, the Fc polypeptide present in a TMMP is an IgG1 Fc polypeptide, or a variant of an IgG1 Fc polypeptide, where variants include naturally occurring variants, non-naturally-occurring variants, and combinations thereof. For example, in some cases, the Fc polypeptide present in a TMMP comprises an amino acid sequence having at least 70%, at least 75%, at least 80%, at least 85%, at least 90%, at least 95%, at least 98%, at least 99%, or 100%, amino acid sequence identity to the human IgG1 Fc polypeptide depicted in FIG. 2C; where the Ig Fc polypeptide comprises a Glu at position 136 and a Met at position 138. As another example, in some cases, the Fc polypeptide present in a TMMP comprises an amino acid sequence having at least 70%, at least 75%, at least 80%, at least 85%, at least 90%, at least 95%, at least 98%, at least 99%, or 100%, amino acid sequence identity to the human IgG1 Fc polypeptide depicted in FIG. 2D; where the Ig Fc polypeptide has Ala at positions 14 and 15; and where the Fc polypeptide comprises a Glu at position 136 and a Met at position 138. In any of the above embodiments, the Ig Fc polypeptide can have an N77 substitution, i.e., the Ig Fc polypeptide can have an amino acid other than Asn at position 77, where in some cases, the Ig Fc polypeptide has an Ala at position 77. In some cases, an Fc polypeptide present in a TMMP comprises the amino acid sequence depicted in FIG. 2C. In some cases, an Fc polypeptide present in a TMMP comprises the amino acid sequence depicted in FIG. 2D.

In some cases, the Fc polypeptide present in a TMMP comprises the amino acid sequence depicted in FIG. 2E (human IgG1 Fc comprising an L234F substitution, an L235E substitution, and a P331S substitution; where L234 corresponds to amino acid 14 of the amino acid sequence depicted in FIG. 2A; L235 corresponds to amino acid 15 of the amino acid sequence depicted in FIG. 2E; and P331 corresponds to amino acid 111 of the amino acid sequence depicted in FIG. 2E). In some cases, the Fc polypeptide present in a TMMP comprises the amino acid sequence depicted in FIG. 2F, comprising an N279A substitution (N77A of the amino acid sequence depicted in FIG. 2F). Substitutions at N297 lead to the removal of carbohydrate modifications and result antibody sequences with reduced complement component 1q (“C1q”) binding compared to the wild-type protein, and accordingly a reduction in complement-dependent cytotoxicity (CDC). In some cases, the Fc polypeptide present in a TMMP comprises a substitution at K322. K322 (e.g., K322A) substitutions shows a substantial reduction in reduction in FcγR binding affinity and a reduction in antibody-dependent cell-mediated cytotoxicity (ADCC), with the C1q binding and CDC functions substantially or completely eliminated.

In some cases, the Fc polypeptide comprises an amino acid sequence having at least 70%, at least 75%, at least 80%, at least 85%, at least 90%, at least 95%, at least 98%, at least 99%, or 100%, amino acid sequence identity to the human IgG2 Fc polypeptide depicted in FIG. 2G; e.g., the Fc polypeptide comprises an amino acid sequence having at least 70%, at least 75%, at least 80%, at least 85%, at least 90%, at least 95%, at least 98%, at least 99%, or 100%, amino acid sequence identity to amino acids 99-325 of the human IgG2 Fc polypeptide depicted in FIG. 2G (e.g., where the Ig Fc polypeptide has a length of about 227 amino acids). In some cases, the Fc polypeptide comprises an amino acid sequence having at least 70%, at least 75%, at least 80%, at least 85%, at least 90%, at least 95%, at least 98%, at least 99%, or 100%, amino acid sequence identity to the human IgG3 Fc polypeptide depicted in FIG. 2H; e.g., the Fc polypeptide comprises an amino acid sequence having at least 70%, at least 75%, at least 80%, at least 85%, at least 90%, at least 95%, at least 98%, at least 99%, or 100%, amino acid sequence identity to amino acids 19-246 of the human IgG3 Fc polypeptide depicted in FIG. 2H (e.g., where the Ig Fc polypeptide has a length of about 228 amino acids). In some cases, the Fc polypeptide comprises an amino acid sequence having at least 70%, at least 75%, at least 80%, at least 85%, at least 90%, at least 95%, at least 98%, at least 99%, or 100%, amino acid sequence identity to the human IgM Fc polypeptide depicted in FIG. 2J; e.g., the Fc polypeptide comprises an amino acid sequence having at least 70%, at least 75%, at least 80%, at least 85%, at least 90%, at least 95%, at least 98%, at least 99%, or 100%, amino acid sequence identity to amino acids 1-276 to the human IgM Fc polypeptide depicted in FIG. 2J. In some cases, the Fc polypeptide comprises an amino acid sequence having at least 70%, at least 75%, at least 80%, at least 85%, at least 90%, at least 95%, at least 98%, at least 99%, or 100%, amino acid sequence identity to the human IgA Fc polypeptide depicted in FIG. 2K; e.g., the Fc polypeptide comprises an amino acid sequence having at least 70%, at least 75%, at least 80%, at least 85%, at least 90%, at least 95%, at least 98%, at least 99%, or 100%, amino acid sequence identity to amino acids 1-234 to the human IgA Fc polypeptide depicted in FIG. 2K.

In some cases, the Fc polypeptide comprises an amino acid sequence having at least 70%, at least 75%, at least 80%, at least 85%, at least 90%, at least 95%, at least 98%, at least 99%, or 100%, amino acid sequence identity to the human IgG4 Fc polypeptide depicted in FIG. 2M. In some cases, the Fc polypeptide comprises an amino acid sequence having at least 70%, at least 75%, at least 80%, at least 85%, at least 90%, at least 95%, at least 98%, at least 99%, or 100%, amino acid sequence identity to amino acids 100 to 327 of the human IgG4 Fc polypeptide depicted in FIG. 2M (e.g., where the Ig Fc polypeptide has a length of about 228 amino acids).

In some cases, the IgG4 Fc polypeptide comprises the following amino acid sequence:

(SEQ ID NO: 936)
PPCPSCPAPEFLGGPSVFLFPPKPKDTLMISRTPEVTCVVVDVSQEDPEV
QFNWYVDGVEVHNAKTKPREEQFNSTYRVVSVLTVLHQDWLNGKEYKCKV
SNKGLPSSIEKTISKAKGQPREPQVYTLPPSQEEMTKNQVSLTCLVKGFY
PSDIAVEWESNGQPENNYKTTPPVLDSDGSFFLYSRLTVDKSRWQEGNVF
SCSVMHEALHNHYTQKSLSLSPG.

### Linkers

As discussed above, a TMMP can include one or more peptide linkers, i.e., a linker comprising a contiguous stretch of two or more amino acids, where the one or more linkers are between one or more components of a TMMP. For example, a TMMP can include one or more peptide linkers, i.e., a linker comprising a contiguous stretch of two or more amino acids, where the one or more linkers are between one or more of: i) an MHC class I heavy chain polypeptide and an Ig Fc polypeptide; ii) a MOD and an MHC class I polypeptide; iii) a first MOD and a second MOD; iv) a peptide and an MHC class I polypeptide; v) a peptide and a β2M polypeptide; vi) a TTP and an MHC class I heavy chain polypeptide; vii) a TTP and an Ig Fc polypeptide; and viii) a TTP and a MOD.

As used herein, the phrase “an optional peptide linker between any two of the components of a TMMP” refers to a peptide linker between any two adjacent polypeptides within the TMMP. For example, as used herein, the phrase “an optional peptide linker between any two of the components of a TMMP” refers to a peptide linker between one or more of:) an MHC class I heavy chain polypeptide and an Ig Fc polypeptide; ii) a MOD and an MHC class I polypeptide; iii) a first MOD and a second MOD; iv) a peptide and an MHC class I polypeptide; v) a peptide and a β2M polypeptide; vi) a TTP and an MHC class I heavy chain polypeptide; vii) a TTP and an Ig Fc polypeptide; and viii) a TTP and a MOD. As discussed below, linkers may be: a) a flexible peptide linker, including a short flexible peptide linker; or b) a rigid peptide linker.

Suitable linkers (also referred to as “spacers”) can be readily selected and can be of any of a number of suitable lengths, such as from 1 amino acid to 25 amino acids, from 3 amino acids to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids. A suitable linker can be 1, 2, 3, 4, 5, 6, 7, 8, 9, 10, 11, 12, 13, 14, 15, 16, 17, 18, 19, 20, 21, 22, 23, 24, or 25 amino acids in length. In some cases, a linker has a length of from 25 amino acids to 50 amino acids, e.g., from 25 to 30, from 30 to 35, from 35 to 40, from 40 to 45, or from 45 to 50 amino acids in length.

### Flexible Peptide Linkers

Exemplary flexible peptide linkers include glycine polymers (G)ₙ (SEQ ID NO:946), glycine-serine polymers (including, for example, (GS)ₙ (SEQ ID NO:937), (GSGGS)ₙ (SEQ ID NO:938), (GGGGS)n (SEQ ID NO:934), and (GGGS)ₙ (SEQ ID NO:939), where n is an integer of at least one and can be an integer from 1 to 10), glycine-alanine polymers, alanine-serine polymers, and other flexible peptide linkers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains (see Scheraga, Rev. Computational Chem. 11173-142 (1992)). Exemplary linkers can comprise amino acid sequences including, but not limited to, GGSG (SEQ ID NO:940), GGSGG (SEQ ID NO:941), GSGSG (SEQ ID NO:942), GSGGG (SEQ ID NO:943), GGGSG (SEQ ID NO:944), GSSSG (SEQ ID NO:945), and the like.

Exemplary flexible peptide linkers include, e.g., (GGGGS)n (SEQ ID NO:934); also referred to as a “G4S” linker), where n is 1, 2, 3, 4, 5, 6, 7, 8, 9, or 10. In some cases, a linker comprises the amino acid sequence (GGGGS)n (SEQ ID NO:934), where n is 1, 2, 3, 4, 5, 6, 7, 8, 9 or 10. In some cases, a linker comprises the amino acid sequence (GGGGS)n (SEQ ID NO:947), where n is 2. In some cases, a linker comprises the amino acid sequence (GGGGS)n (SEQ ID NO:875), where n is 3. In some cases, a linker comprises the amino acid sequence (GGGGS)n (SEQ ID NO:876), where n is 4. In some cases, a linker comprises the amino acid sequence (GGGGS)n (SEQ ID NO:948), where n is 7. In some cases, a linker comprises the amino acid sequence AAAGG (SEQ ID NO:874). Also suitable is a linker having the amino acid sequence AAAGG (SEQ ID NO:874). In TMMPs of this disclosure, the β2M polypeptide can be connected to the MHC heavy chain polypeptide by a (GGGGS)n (SEQ ID NO:934) linker, where n is 1, 2, 3, 4, 5, 6, 7, 8, 9, or 10, e.g., where n=3, n=4, or n=7.

As used in this disclosure, a “short flexible peptide linker” means a flexible peptide linker that comprises fewer than 15 amino acids, i.e., from 2-14 amino acids. For example, a short flexible peptide linker can comprise from 2-4 amino acids, from 2-5 amino acids, from 3-6 amino acids, from 4-8 amino acids, from 5-10 amino acids, or from 10-14 amino acids. Within this range includes flexible peptide linkers comprising 2, 3, 4, 5, 6, 7, 8, 9, 10, 11, 12, 13 or 14 amino acids.

### Rigid Peptide Linkers

In some cases, a peptide linker is a rigid peptide linker. As used herein, the term “rigid peptide linker” refers to a linker comprising a contiguous stretch of two or more amino acids that effectively separates protein domains by maintaining a substantially fixed distance/spatial separation between the domains, thereby reducing or substantially eliminating unfavorable interactions between such domains. For example, rigid linkers may be interposed when either the MOD(s) or the TTP are in Position 1 and/or in Position 3 discussed above. Rigid peptide linkers are known in the art and generally adopt a relatively well-defined conformation when in solution. Rigid peptide linkers include those which have a particular secondary and/or tertiary structure in solution; and are typically of a length sufficient to confer secondary or tertiary structure to the linker. Rigid peptide linkers include peptide linkers rich in proline, and peptide linkers having an inflexible helical structure, such as an α-helical structure. Rigid peptide linkers are described in, for example, Chen et al. (2013) Adv. Drug Deliv. Rev. 65:1357; and Klein et al. (2014) Protein Engineering, Design & Selection 27:325.

Examples of rigid peptide linkers include, e.g., (EAAAK)n (SEQ ID NO:949), A(EAAAK)nA (SEQ ID NO:950), A(EAAAK)nALEA(EAAAK)nA (SEQ ID NO:951), (Lys-Pro)n (SEQ ID NO:952), (Glu-Pro)n (SEQ ID NO:953), (Thr-Pro-Arg)n (SEQ ID NO:954), and (Ala-Pro)n (SEQ ID NO:955) where n is an integer from 1 to 20 (e.g., n is 1, 2, 3, 4, 5, 6, 7, 8, 9, 10, 11, 12, 13, 14, 15, 16, 17, 18, 19, or 20). Non-limiting examples of suitable rigid peptide linkers comprising EAAAK (SEQ ID NO:956) include EAAAK (SEQ ID NO:956), (EAAAK)₂ (SEQ ID NO:958), (EAAAK)₃ (SEQ ID NO:959), A(EAAAK)₄ALEA(EAAAK)₄A (SEQ ID NO:960), and AEAAAKEAAAKA (SEQ ID NO:961). Non-limiting examples of suitable rigid peptide linkers comprising (AP)n include APAP (SEQ ID NO:962; also referred to herein as “(AP)2”); APAPAPAP (SEQ ID NO:963; also referred to herein as “(AP)4”); APAPAPAPAPAP (SEQ ID NO:964; also referred to herein as “(AP)6”); APAPAPAPAPAPAPAP (SEQ ID NO:965; also referred to herein as “(AP)8”); and APAPAPAPAPAPAPAPAPAP (SEQ ID NO:955; also referred to herein as “(AP)10”). Non-limiting examples of suitable rigid peptide linkers comprising (KP)n include KPKP (SEQ ID NO:967; also referred to herein as “(KP)2”); KPKPKPKP (SEQ ID NO:968; also referred to herein as “(KP)4”); KPKPKPKPKPKP (SEQ ID NO:969; also referred to herein as “(KP)6”); KPKPKPKPKPKPKPKP (SEQ ID NO:970; also referred to herein as “(KP)8”); and KPKPKPKPKPKPKPKPKPKP (SEQ ID NO:952; also referred to herein as “(KP)10”). Non-limiting examples of suitable rigid peptide linkers comprising (EP)n include EPEP (SEQ ID NO:972; also referred to herein as “(EP)2”); EPEPEPEP (SEQ ID NO:973; also referred to herein as “(EP)4”); EPEPEPEPEPEP (SEQ ID NO:974; also referred to herein as “(EP)6”); EPEPEPEPEPEPEPEP (SEQ ID NO:975; also referred to herein as “(EP)8”); and EPEPEPEPEPEPEPEPEPEP (SEQ ID NO:953; also referred to herein as “(EP)10”).

### Cysteine-Containing Linkers

As noted above, in some cases, a linker peptide in a first polypeptide chain of a TMMP can include a cysteine residue that can form an interchain disulfide bond with a cysteine residue present in a second polypeptide chain. For example, as discussed above, in some cases a TMMP, or a dimerized TMMP such as a homodimer, comprises a linker between the peptide epitope and the β2M polypeptide that includes a cysteine residue that forms an interchain disulfide bond with a cysteine residue in the MHC class I heavy chain polypeptide present in the other chain of the TMMP. For example, in some cases, where a TMMP, or a dimerized TMMP such as a homodimer, comprises a cysteine-containing linker between the peptide epitope and the β2M polypeptide, the cysteine residue in the linker forms an interchain disulfide bond with a cysteine residue at amino acid 236 (e.g., formed by an A236C substitution) in the MHC class I heavy chain polypeptide present in the TMMP.

In some cases, the peptide linker between the SARS-CoV-2 peptide and the β2M polypeptide comprises the amino acid sequence GCGGS (SEQ ID NO:977). In some cases, the peptide linker between the SARS-CoV-2 peptide and the β2M polypeptide comprises the amino acid sequence GCGGS(GGGGS)n (SEQ ID NO:978), where n is 1, 2, 3, 4, 5, 6, 7, 8, 9, or 10; e.g., 1, 2, or 3. In some cases, the peptide linker between the peptide and the β2M polypeptide comprises the amino acid sequence GCGGS(GGGGS)n (SEQ ID NO:882), where n is 2.

In some cases, the peptide linker between the SARS-CoV-2 peptide and the β2M polypeptide comprises the amino acid sequence CGGGS (SEQ ID NO:979). In some cases, the peptide linker comprises the amino acid sequence CGGGS(GGGGS)n (SEQ ID NO:980), where n is 1, 2, 3, 4, 5, 6, 7, 8, 9, or 10; e.g., 1, 2, or 3.

In some cases, the peptide linker between the SARS-CoV-2 peptide and the β2M polypeptide comprises the amino acid sequence GGCGS (SEQ ID NO:981). In some cases, the peptide linker comprises the amino acid sequence GGCGS(GGGGS)n (SEQ ID NO:982), where n is 1, 2, 3, 4, 5, 6, 7, 8, 9 or 10, e.g., 1, 2, or 3.

In some cases, the peptide linker between the SARS-CoV-2 peptide and the β2M polypeptide comprises the amino acid sequence GGGCS (SEQ ID NO:983). In some cases, the peptide linker comprises the amino acid sequence GGGCS(GGGGS)n (SEQ ID NO:984), where n is 1, 2, 3, 4, 5, 6, 7, 8, 9, or 10, e.g., 1, 2, or 3.

In some cases, the peptide linker between the SARS-CoV-2 peptide and the β2M polypeptide comprises the amino acid sequence GGGGC (SEQ ID NO:985). In some cases, the peptide linker comprises the amino acid sequence GGGGC(GGGGS)n (SEQ ID NO:986), where n is 1, 2, 3, 4, 5, 6, 7, 8, 9, or 10, e.g., 1, 2, or 3.

### Linkers Between the TTP and MHC Class I Heavy Chain Polypeptide

As discussed below, it has been found that the cytotoxic effect of TMMPs is greater when a TTP is positioned N-terminal to the MHC heavy chain polypeptide and the linker between them is no more than about 12 amino acids in length, e.g., no more than 10 amino acids in length (e.g., a (GGGGS)₂ (SEQ ID NO:947) linker), no more than 8 amino acids in length, no more than 7 amino acids in length, no more than 6 amino acids in length, or no more than 5 amino acids in length, (e.g., a GGGGS (SEQ ID NO:873) linker). In some cases, the linker between them is no more than about 12 amino acids in length. In some cases, the linker between them is no more than 10 amino acids in length (e.g., a (GGGGS)₂ (SEQ ID NO:947) linker). In some cases, the linker between them is no more than 8 amino acids in length. In some cases, the linker between them is no more than 7 amino acids in length. In some cases, the linker between them is no more than 6 amino acids in length. In some cases, the linker between them is no more than 5 amino acids in length, (e.g., a GGGGS (SEQ ID NO:873) linker).

### Dimerized Tmmps

As noted above, in some cases, a TMMP can form dimers. That is, the present disclosure provides a polypeptide comprising a dimer of two TMMPs. The present disclosure thus provides a protein that is a dimerized TMMP comprising two TMMPs that are covalently linked to each other. The covalent linkage of the dimer can be one or more disulfide bonds between an Ig Fc polypeptide in the first TMMP and an Ig Fc polypeptide in the second TMMP. As but one example, the Ig Fc can be a variant of a human IgG1 Fc polypeptide, which variant has a substantially reduced ability to effect complement-dependent cytotoxicity (CDC) or antibody-dependent cell cytotoxicity (ADCC) (e.g., the human IgG1 Fc polypeptide of FIG. 2B or FIG. 2D). When the TMMP comprises an Ig Fc polypeptide, the TMMP typically will self-assemble into a dimer by spontaneously forming disulfide bonds with the IgG1 Fc polypeptide of another TMMP. Thus, e.g., the Ig Fc polypeptides in the first TMMP and the second TMMP can be linked to one another by one or more disulfide bonds. In many cases, the two TMMPs will be identical to one another in amino acid sequence and comprise Ig Fc polypeptides that spontaneously form one or more disulfide bonds, thereby forming a dimerized TMMP that is a homodimer.

Accordingly, the present disclosure provides a protein comprising: a) a first TMMP; and b) a second TMMP, which optionally may be identical to the first TMMP, where the first and second TMMPs are covalently linked to one another. The covalent linkage can be a disulfide bond between an Ig Fc polypeptide in the first TMMP and an Ig Fc polypeptide in the second TMMP.

If desired, the Ig Fc polypeptides of each TMMP can comprise interspecific dimerization sequences, e.g., “Knob-in-Hole” sequences that permit two different TMMPs to selectively dimerize. Interspecific binding sequences favor formation of heterodimers with their cognate polypeptide sequence (i.e., the interspecific sequence and its counterpart interspecific sequence), particularly those based on Ig Fc sequence variants. Such interspecific polypeptide sequences include Knob-in-Hole, and Knob-in-Hole sequences that facilitate the formation of one or more disulfide bonds. For example, one interspecific binding pair comprises a T366Y and Y407T mutant pair in the CH3 domain interface of IgG1, or the corresponding residues of other immunoglobulins. See Ridgway et al., Protein Engineering 9:7, 617-621 (1996). A second interspecific binding pair involves the formation of a knob by a T366W substitution, and a hole by the triple substitutions T366S, L368A and Y407V on the complementary Ig Fc sequence. See Xu et al. mAbs 7:1, 231-242 (2015). Another interspecific binding pair has a first Fc polypeptide with Y349C, T366S, L368A, and Y407V substitutions and a second Ig Fc polypeptide with S354C, and T366W substitutions (disulfide bonds can form between the Y349C and the S354C). See, e.g., Brinkmann and Konthermann, mAbs 9:2, 182-212 (2015). Ig Fc polypeptide sequences, either with or without knob-in-hole modifications, can be stabilized by the formation of disulfide bonds between the Ig Fc polypeptides (e.g., the hinge region disulfide bonds). Thus, in some cases, a dimerized TMMP can be a heterodimer, comprising two TMMP chains that are not identical in amino acid sequence.

Interspecific dimerization sequences also may be employed to enable TMMPs to be linked to non-TMMP molecules that can provide additional functionality to the TMMP. For example, a TMMP could be linked to a molecule that comprise polypeptides (e.g., antibodies or binding fragments thereof such as scFvs) that bind to cancer-associated antigens, thereby enabling the TMMP to localize to tissues comprising the cancer-associated antigen.

### Tumor-Targeting Polypeptides (TTPs)

As discussed above, a TMMP includes, in the first and/or the second polypeptide, a tumor-targeting polypeptide (TTP), i.e., a polypeptide specific for a cancer-associated epitope. A “cancer-associated” epitope is an epitope that is present in a cancer-associated antigen. In some cases, a TTP is an antibody. In some cases, a TTP is a single-chain T-cell receptor (scTCR).

### Targets

In some cases, a TTP present in a TMMP targets a cancer-associated antigen. In some cases, the target of a TTP is a peptide/HLA (pHLA) complex on the surface of a cancer cell, where the peptide can be a cancer-associated peptide (e.g., a peptide fragment of a cancer-associated antigen).

### Cancer-Associated Antigens

Cancer-associated antigens that can be targeted with a tumor-targeting polypeptide present in a TMMP include, e.g., NY-ESO (New York Esophageal Squamous Cell Carcinoma 1), MART-1 (melanoma antigen recognized by T cells 1, also known as Melan-A), HPV (human papilloma virus) E6, BCMA (B-cell maturation antigen), CD123, CD133, CD171, CD19, CD20, CD22, CD30, CD33, CD38, CD138, CEA (carcinoembryonic antigen), EGFR (epidermal growth factor receptor), EGFRvIII (epidermal growth factor receptor variant III), EpCAM (epithelial cell adhesion molecule), EphA2 (ephrin type-A receptor 2), disialoganglioside GD2, GPC3 (glypican-3), HER2, IL13Ralpha2 (Interleukin 13 receptor subunit alpha-2), LeY (a difucosylated type 2 blood group-related antigen), MAGE-A3 (melanoma-associated antigen 3), melanoma glycoprotein, mesothelin, MUC1 (mucin 1), MUC16 (mucin-16), myelin, NKG2D (Natural Killer Group 2D) ligands, PSMA (prostate specific membrane antigen), and RORI (type I receptor tyrosine kinase-like orphan receptor).

Cancer-associated antigens that can be targeted with a TTP present in a TMMP include, but are not limited to, 17-1A-antigen, alpha-fetoprotein (AFP), alpha-actinin-4, A3, antigen specific for A33 antibody, ART-4, B7, Ba 733, BAGE, bcl-2, bcl-6, BCMA, BrE3-antigen, CA125, CAMEL, CAP-1, carbonic anhydrase IX (CAIX), CASP-8/m, CCL19, CCL21, CD1, CD1a, CD2, CD3, CD4, CD5, CD8, CD11A, CD14, CD15, CD16, CD18, CD19, CD20, CD21, CD22, CD23, CD25, CD29, CD30, CD32b, CD33, CD37, CD38, CD40, CD40L, CD44, CD45, CD46, CD52, CD54, CD55, CD59, CD64, CD66a-e, CD67, CD70, CD70L, CD74, CD79a, CD79b, CD80, CD83, CD95, CD123, CD126, CD132, CD133, CD138, CD147, CD154, CD171, CDC27, CDK-4/m, CDKN2A, CEA, CEACAM5, CEACAM6, claudin (e.g., claudin-1, claudin-10, claudin-18 (e.g., claudin-18, isoform 2)), complement factors (such as C3, C3a, C3b, C5a and C5), colon-specific antigen-p (CSAp), c-Met, CTLA-4, CXCR4, CXCR7, CXCL12, DAM, Dickkopf-related protein (DKK), ED-B fibronectin, epidermal growth factor receptor (EGFR), EGFRvIII, EGP-1 (TROP-2), EGP-2, ELF2-M, Ep-CAM, EphA2, EphA3, fibroblast activation protein (FAP), fibroblast growth factor (FGF), Flt-1, Flt-3, folate binding protein, folate receptor, G250 antigen, gangliosides (such as GC2, GD3 and GM2), GAGE, GD2, gp100, GPC3, GRO-13, HLA-DR, HM1.24, human chorionic gonadotropin (HCG) and its subunits, HER2, HER3, HMGB-1, hypoxia inducible factor (HIF-1), HIF-1a, HSP70-2M, HST-2, Ia, IFN-gamma, IFN-alpha, IFN-beta, IFN-X, IL-4R, IL-6R, IL-13R, IL13Ralpha2, IL-15R, IL-17R, IL-18R, IL-2, IL-6, IL-8, IL-12, IL-15, IL-17, IL-18, IL-23, IL-25, ILGF, ILGF-1R, insulin-like growth factor-1 (IGF-1), IGF-1R, integrin aV 3, integrin a51, KC4-antigen, killer-cell immunoglobulin-like receptor (KIR), Kras, KS-1-antigen, KS1-4, LDR/FUT, Le^{gamma}, macrophage migration inhibitory factor (MIF), MAGE, MAGE-3, MART-1, MART-2, mCRP, MCP-1, melanoma glycoprotein, mesothelin, MIP-1A, MIP-1B, MIF, mucins (such as MUC1, MUC2, MUC3, MUC4, MUC5ac, MUC13, MUC16, MUM-1/2 and MUM-3), NCA66, NCA95, NCA90, Nectin-4, NY-ESO-1, PAM4 antigen, pancreatic cancer mucin, PD-1, PD-L1, PD-1 receptor, placental growth factor, p53, PLAGL2, prostatic acid phosphatase, PSA, PRAME, PSMA, P1GF, RSS, RANTES, SAGE, 5100, survivin, survivin-2B, T101, TAC, TAG-72, tenascin, Thomson-Friedenreich antigens, Tn antigen, TNF-alpha, tumor necrosis antigens, TRAG-3, TRAIL receptors, vascular endothelial growth factor (VEGF), VEGF receptor (VEGFR) and WT-1.

In some cases, the cancer-associated antigen is an antigen associated with a hematological cancer. Examples of such antigens include, but are not limited to, BCMA, C5, CD19, CD20, CD22, CD25, CD30, CD33, CD38, CD40, CD45, CD52, CD56, CD66, CD74, CD79a, CD79b, CD80, CD138, CTLA-4, CXCR4, DKK, EphA3, GM2, HLA-DR beta, integrin αVβ3, IGF-R1, IL6, KIR, PD-1, PD-L1, TRAILR1, TRAILR2, transferrin receptor, and VEGF. In some cases, the cancer-associated antigen is an antigen expressed by malignant B cells, such as CD19, CD20, CD22, CD25, CD38, CD40, CD45, CD74, CD80, CTLA-4, IGF-R1, IL6, PD-1, TRAILR2, or VEGF.

In some cases, the cancer-associated antigen is an antigen associated with a solid tumor. Examples of such antigens include, but are not limited to, CAIX, cadherins, CEA, c-MET, CTLA-4, EGFR family members, EpCAM, EphA3, FAP, folate-binding protein, FR-alpha, gangliosides (such as GC2, GD3 and GM2), HER2, HER3, IGF-1R, integrin αVβ3, integrin α5β1, Le^{gamma}, Liv1, mesothelin, mucins, NaPi2b, PD-1, PD-L1, PD-1 receptor, pgA33, PSMA, RANKL, RORI, TAG-72, tenascin, TRAILR1, TRAILR2, VEGF, VEGFR, and others listed above.

### Peptide/HLA Complexes

In some cases, the target of a TTP is a peptide/HLA (pHLA) complex on the surface of a cancer cell, where the peptide can be a cancer-associated peptide (e.g., a peptide fragment of a cancer-associated antigen). Cancer-associated peptides are known in the art. In some cases, a cancer-associated peptide is bound to an HLA complex comprising an HLA-A*0201 heavy chain and a β2M polypeptide.

In some cases, the epitope present in the pHLA on the surface of a cancer cell is bound to an HLA complex comprising an HLA heavy chain such as HLA-A*0101, A*0201, A*0301, A*1101, A*2301, A*2402, A*2407, A*3303, and/or A*3401. In some cases, the epitope present in the pHLA on the surface of a cancer cell is bound to an HLA complex comprising an HLA heavy chain such as HLA-B*0702, B*0801, B*1502, B*3802, B*4001, B*4601, and/or B*5301. In some cases, the epitope present in the pHLA on the surface of a cancer cell is bound to an HLA complex comprising an HLA heavy chain such as C*0102, C*0303, C*0304, C*0401, C*0602, C*0701, C*702, C*0801, and/or C*1502.

In some cases, the epitope is a cancer-associated epitope of any one of the following cancer-associated antigens: a MUC1 polypeptide, an LMP2 polypeptide, an epidermal growth factor receptor (EGFR) vIII polypeptide, a HER-2/neu polypeptide, a melanoma antigen family A, 3 (MAGE A3) polypeptide, a p53 polypeptide, a mutant p53 polypeptide, an NY-ESO-1 polypeptide, a folate hydrolase (prostate-specific membrane antigen; PSMA) polypeptide, a carcinoembryonic antigen (CEA) polypeptide, a claudin polypeptide (e.g., claudin-1, claudin-10, claudin-18 (e.g., claudin-18, isoform 2)), a Nectin-4 polypeptide, a melanoma antigen recognized by T-cells (melanA/MART1) polypeptide, a Ras polypeptide, a gp100 polypeptide, a proteinase3 (PR1) polypeptide, a bcr-abl polypeptide, a tyrosinase polypeptide, a survivin polypeptide, a prostate specific antigen (PSA) polypeptide, an hTERT polypeptide, a sarcoma translocation breakpoints polypeptide, a synovial sarcoma X (SSX) breakpoint polypeptide, an EphA2 polypeptide, an acid phosphatase, prostate (PAP) polypeptide, a melanoma inhibitor of apoptosis (ML-IAP) polypeptide, an epithelial cell adhesion molecule (EpCAM) polypeptide, an ERG (TMPRSS2 ETS fusion) polypeptide, a NA17 polypeptide, a paired-box-3 (PAX3) polypeptide, an anaplastic lymphoma kinase (ALK) polypeptide, an androgen receptor polypeptide, a cyclin B1 polypeptide, an N-myc proto-oncogene (MYCN) polypeptide, a Ras homolog gene family member C (RhoC) polypeptide, a tyrosinase-related protein-2 (TRP-2) polypeptide, a mesothelin polypeptide, a prostate stem cell antigen (PSCA) polypeptide, a melanoma associated antigen-1 (MAGE A1) polypeptide, a cytochrome P450 1B1 (CYPIB1) polypeptide, a placenta-specific protein 1 (PLAC1) polypeptide, a BORIS polypeptide (also known as CCCTC-binding factor or CTCF), an ETV6-AML polypeptide, a breast cancer antigen NY-BR-1 polypeptide (also referred to as ankyrin repeat domain-containing protein 30A), a regulator of G-protein signaling (RGS5) polypeptide, a squamous cell carcinoma antigen recognized by T-cells (SART3) polypeptide, a carbonic anhydrase IX polypeptide, a paired box-5 (PAX5) polypeptide, an OY-TES1 (testis antigen; also known as acrosin binding protein) polypeptide, a sperm protein 17 polypeptide, a lymphocyte cell-specific protein-tyrosine kinase (LCK) polypeptide, a high molecular weight melanoma associated antigen (HMW-MAA), an A-kinase anchoring protein-4 (AKAP-4), a synovial sarcoma X breakpoint 2 (SSX2) polypeptide, an X antigen family member 1 (XAGE1) polypeptide, a B7 homolog 3 (B7H3; also known as CD276) polypeptide, a legumain polypeptide (LGMN1; also known as asparaginyl endopeptidase), a tyrosine kinase with Ig and EGF homology domains-2 (Tie-2; also known as angiopoietin-1 receptor) polypeptide, a P antigen family member 4 (PAGE4) polypeptide, a vascular endothelial growth factor receptor 2 (VEGF2) polypeptide, a MAD-CT-1 polypeptide, a fibroblast activation protein (FAP) polypeptide, a platelet derived growth factor receptor beta (PDGFO) polypeptide, a MAD-CT-2 polypeptide, a Fos-related antigen-1 (FOSL) polypeptide; a human papilloma virus (HPV) antigen; an alpha-feto protein (AFP) antigen; and a Wilms tumor-1 (WTi) antigen.

For example, in some cases, a TTP present in a TMMP binds to: a) a WT-1 peptide bound to an HLA complex comprising an HLA heavy chain (e.g., an HLA-A*0201 heavy chain or an HLA-A*2402 heavy chain) and a β2M polypeptide; b) an HPV peptide bound to an HLA complex comprising a class I HLA heavy chain and a β2M polypeptide; c) a mesothelin peptide bound to an HLA complex comprising a class I HLA heavy chain and a β2M polypeptide; d) a Her2 peptide bound to an HLA complex comprising a class I HLA heavy chain and a β2M polypeptide; or e) a BCMA peptide bound to an HLA complex comprising a class I HLA heavy chain and a β2M polypeptide.

In some cases, a cancer-associated peptide is a peptide of a mesothelin polypeptide having at least 90%, at least 95%, at least 98%, at least 99%, or 100%, amino acid sequence identity to the following mesothelin amino acid sequence:
LAGE TGQEAAPLDG VLANPPNISS LSPRQLLGFP CAEVSGLSTE RVRELAVALA QKNVKLSTEQ LRCLAHRLSE PPEDLDALPL DLLLFLNPDA FSGPQACTRF FSRITKANVD LLPRGAPERQ RLLPAALACW GVRGSLLSEA DVRALGGLAC DLPGRFVAES AEVLLPRLVS CPGPLDQDQQ EAARAALQGG GPPYGPPSTW SVSTMDALRG LLPVLGQPII RSIPQGIVAA WRQRSSRDPS WRQPERTILR PRFRREVEKT ACPSGKKARE IDESLIFYKK WELEACVDAA LLATQMDRVN AIPFTYEQLD VLKHKLDELY PQGYPESVIQ HLGYLFLKMS PEDIRKWNVT SLETLKALLE VNKGHEMSPQ VATLIDRFVK GRGQLDKDTL DTLTAFYPGY LCSLSPEELS SVPPSSIWAV RPQDLDTCDP RQLDVLYPKA RLAFQNMNGS EYFVKIQSFL GGAPTEDLKA LSQQNVSMDL ATFMKLRTDA VLPLTVAEVQ KLLGPHVEGL KAEERHRPVR DWILRQRQDD LDTLGLGLQG GIPNGYLVLD LSMQEALSGT PCLLGPGPVL TVLALLLAST LA (SEQ ID NO:987). For example, a mesothelin peptide present in a pHLA complex can be: i) KLLGPHVEGL (SEQ ID NO:988); ii) AFYPGYLCSL (SEQ ID NO:989), which can bind HLA-A*2402/02M; iii) VLPLTVAEV (SEQ ID NO:990); iv) ELAVALAQK (SEQ ID NO:991); v) ALQGGGPPY (SEQ ID NO:992); vi) FYPGYLCSL (SEQ ID NO:993); vii) LYPKARLAF (SEQ ID NO:994); viii) LLFLLFSLGWVGPSR (SEQ ID NO:995); ix) VNKGHEMSPQAPRRP (SEQ ID NO:996); x) FMKLRTDAVLPLTVA (SEQ ID NO:997); or xi) DAALLATQMD (SEQ ID NO:998).

In some cases, a cancer-associated peptide is a peptide of a Her2 polypeptide having at least 90%, at least 95%, at least 98%, at least 99%, or 100%, amino acid sequence identity to the following Her2 (receptor tyrosine-protein kinase erbB2) amino acid sequence:

(SEQ ID NO: 999)

MELAALCRWG LLLALLPPGA ASTQVCTGTD MKLRLPASPE
THLDMLRHLY QGCQVVQGNL ELTYLPTNAS LSFLQDIQEV
QGYVLIAHNQ VRQVPLQRLR IVRGTQLFED NYALAVLDNG
DPLNNTTPVT GASPGGLREL QLRSLTEILK GGVLIQRNPQ
LCYQDTILWK DIFHKNNQLA LTLIDTNRSR ACHPCSPMCK
GSRCWGESSE DCQSLTRTVC AGGCARCKGP LPTDCCHEQC
AAGCTGPKHS DCLACLHFNH SGICELHCPA LVTYNTDTFE
SMPNPEGRYT FGASCVTACP YNYLSTDVGS CTLVCPLHNQ
EVTAEDGTQR CEKCSKPCAR VCYGLGMEHL REVRAVTSAN
IQEFAGCKKI FGSLAFLPES FDGDPASNTA PLQPEQLQVF
ETLEEITGYL YISAWPDSLP DLSVFQNLQV IRGRILHNGA
YSLTLQGLGI SWLGLRSLRE LGSGLALIHH NTHLCFVHTV
PWDQLFRNPH QALLHTANRP EDECVGEGLA CHQLCARGHC
WGPGPTQCVN CSQFLRGQEC VEECRVLQGL PREYVNARHC
LPCHPECQPQ NGSVTCFGPE ADQCVACAHY KDPPFCVARC
PSGVKPDLSY MPIWKFPDEE GACQPCPINC THSCVDLDDK
GCPAEQRASP LTSIISAVVG ILLVVVLGVV FGILIKRRQQ
KIRKYTMRRL LQETELVEPL TPSGAMPNQA QMRILKETEL
RKVKVLGSGA FGTVYKGIWI PDGENVKIPV AIKVLRENTS
PKANKEILDE AYVMAGVGSP YVSRLLGICL TSTVQLVTQL
MPYGCLLDHV RENRGRLGSQ DLLNWCMQIA KGMSYLEDVR
LVHRDLAARN VLVKSPNHVK ITDFGLARLL DIDETEYHAD
GGKVPIKWMA LESILRRRFT HQSDVWSYGV TVWELMTFGA
KPYDGIPARE IPDLLEKGER LPQPPICTID VYMIMVKCWM
IDSECRPRFR ELVSEFSRMA RDPQRFVVIQ NEDLGPASPL
DSTFYRSLLE DDDMGDLVDA EEYLVPQQGF FCPDPAPGAG
GMVHHRHRSS STRNM.

In some cases, a cancer-associated peptide is a peptide of a BCMA polypeptide having at least 90%, at least 95%, at least 98%, at least 99%, or 100%, amino acid sequence identity to the following BCMA amino acid sequence:

(SEQ ID NO: 1000)

MLQMAGQCSQ NEYFDSLLHA CIPCQLRCSS NTPPLTCQRY
CNASVTNSVK GTNAILWTCL GLSLIISLAV FVLMFLLRKI
SSEPLKDEFK NTGSGLLGMA NIDLEKSRTG DEIILPRGLE
YTVEECTCED CIKSKPKVDS DHCFPLPAME EGATILVTTK
TNDYCKSLPA ALSATEIEKS ISAR.

In some cases, a cancer-associated peptide is a peptide of a WT-1 polypeptide having at least 90%, at least 95%, at least 98%, at least 99%, or 100%, amino acid sequence identity to the following WT-1 amino acid sequence:

(SEQ ID NO: 1001)

MDFLLLQDPA STCVPEPASQ HTLRSGPGCL QQPEQQGVRD
PGGIWAKLGA AEASAERLQG RRSRGASGSE PQQMGSDVRD
LNALLPAVPS LGGGGGCALP VSGAAQWAPV LDFAPPGASA
YGSLGGPAPP PAPPPPPPPP PHSFIKQEPS WGGAEPHEEQ
CLSAFTVHFS GQFTGTAGAC RYGPFGPPPP SQASSGQARM
FPNAPYLPSC LESQPAIRNQ GYSTVTFDGT PSYGHTPSHH
AAQFPNHSFK HEDPMGQQGS LGEQQYSVPP PVYGCHTPTD
SCTGSQALLL RTPYSSDNLY QMTSQLECMT WNQMNLGATL
KGHSTGYESD NHTTPILCGA QYRIHTHGVF RGIQDVRRVP
GVAPTLVRSA SETSEKRPFM CAYPGCNKRY FKLSHLQMHS
RKHTGEKPYQ CDFKDCERRF SRSDQLKRHQ RRHTGVKPFQ
CKTCQRKFSR SDHLKTHTRT HTGEKPFSCR WPSCQKKFAR
SDELVRHHNM HQRNMTKLQL AL.

Non-limiting examples of WT-1 peptides include RMFPNAPYL (SEQ ID NO:1002), CMTWNQMN (SEQ ID NO:1003), CYTWNQMNL (SEQ ID NO:1004), CMTWNQMNLGATLKG (SEQ ID NO:1005), WNQMNLGATLKGVAA (SEQ ID NO:1006), CMTWNYMNLGATLKG (SEQ ID NO:1007), WNYMNLGATLKGVAA (SEQ ID NO:1008), MTWNQMNLGATLKGV (SEQ ID NO:1009), TWNQMNLGATLKGVA (SEQ ID NO:1010), CMTWNLMNLGATLKG (SEQ ID NO:1011), MTWNLMNLGATLKGV (SEQ ID NO:1012), TWNLMNLGATLKGVA (SEQ ID NO:1013), WNLMNLGATLKGVAA (SEQ ID NO:1014), MNLGATLK (SEQ ID NO:1015), MTWNYMNLGATLKGV (SEQ ID NO:1016), TWNYMNLGATLKGVA (SEQ ID NO:1017), CMTWNQMNLGATLKGVA (SEQ ID NO:1018), CMTWNLMNLGATLKGVA (SEQ ID NO:1019), CMTWNYMNLGATLKGVA (SEQ ID NO:1020), GYLRNPTAC (SEQ ID NO:1021), GALRNPTAL (SEQ ID NO:1022), YALRNPTAC (SEQ ID NO:1023), GLLRNPTAC (SEQ ID NO:1024), RYRPHPGAL (SEQ ID NO:1025), YQRPHPGAL (SEQ ID NO:1026), RLRPHPGAL (SEQ ID NO:1027), RIRPHPGAL (SEQ ID NO:1028), QFPNHSFKHEDPMGQ (SEQ ID NO:1029), HSFKHEDPY (SEQ ID NO:1030), QFPNHSFKHEDPM (SEQ ID NO:1031), QFPNHSFKHEDPY (SEQ ID NO:1032), KRPFMCAYPGCNK (SEQ ID NO:1033), KRPFMCAYPGCYK (SEQ ID NO:1034), FMCAYPGCY (SEQ ID NO:1035), FMCAYPGCK (SEQ ID NO:1036), KRPFMCAYPGCNKRY (SEQ ID NO:1037), SEKRPFMCAYPGCNK (SEQ ID NO:1038), KRPFMCAYPGCYKRY (SEQ ID NO:1039), NLMNLGATL (SEQ ID NO:1040), and NYMNLGATL (SEQ ID NO:1041).

In some cases, a cancer-associated peptide is a peptide of a human papillomavirus (HPV) polypeptide having at least 90%, at least 95%, at least 98%, at least 99%, or 100%, amino acid sequence identity to an HPV polypeptide. An HPV peptide can be a peptide of an HPV E6 polypeptide or an HPV E7 polypeptide. The HPV epitope can be an epitope of HPV of any of a variety of genotypes, including, e.g., HPV16, HPV18, HPV31, HPV33, HPV35, HPV39, HPV45, HPV51, HPV52, HPV56, HPV58, HPV59, HPV68, HPV73, or HPV82. Non-limiting examples of HPV peptides include: E6 18-26 (KLPQLCTEL; SEQ ID NO:1042); E6 26-34 (LQTTIHDII; SEQ ID NO:1043); E6 49-57 (VYDFAFRDL; SEQ ID NO:1044); E6 52-60 (FAFRDLCIV; SEQ ID NO:1045); E6 75-83 (KFYSKISEY; SEQ ID NO:1046); E6 80-88 (ISEYRHYCY; SEQ ID NO:1047); E7 7-15 (TLHEYMLDL; SEQ ID NO:1048); E7 11-19 (YMLDLQPET; SEQ ID NO:1049); E7 44-52 (QAEPDRAHY; SEQ ID NO:1050); E7 49-57 (RAHYNIVTF (SEQ ID NO:1051); E7 61-69 (CDSTLRLCV; SEQ ID NO:1052); and E7 67-76 (LCVQSTHVDI; SEQ ID NO:1053); E7 82-90 (LLMGTLGIV; SEQ ID NO:1054); E7 86-93 (TLGIVCPI; SEQ ID NO:1055); and E7 92-93 (LLMGTLGIVCPI; SEQ ID NO:1056).

In some cases, a cancer-associated peptide is a peptide of a claudin polypeptide having at least 90%, at least 95%, at least 98%, at least 99%, or 100%, amino acid sequence identity to the following claudin-18 (isoform 2) (CLDN 18.2) amino acid sequence:
MAVTACQGLG FVVSLIGIAG IIAATCMDQW STQDLYNNPV TAVFNYQGLW RSCVRESSGF TECRGYFTLL GLPAMLQAVR ALMIVGIVLG AIGLLVSIFA LKCIRIGSME DSAKANMTLT SGIMFIVSGL CAIAGVSVFA NMLVTNFWMS TANMYTGMGG MVQTVQTRYT FGAALFVGWV AGGLTLIGGV MMCIACRGLA PEETNYKAVS YHASGHSVAY KPGGFKASTG FGSNTKNKKI YDGGARTEDE VQSYPSKHDY V (SEQ ID NO:1057). In some cases, a cancer-associated peptide is a peptide of a claudin polypeptide having the amino acid sequence TEDEVQSYPSKHDYV (SEQ ID NO:1058) (and having a length of about 15 amino acids) or EVQSYPSKHDYV (SEQ ID NO:1059) (and having a length of about 12 amino acids.

In some cases, a cancer-associated peptide is a peptide of a trophoblast cell-surface antigen-2 (Trop-2) polypeptide. Trop-2 (also known as epithelial glycoprotein-1, gastrointestinal tumor-associated antigen GA733-1, membrane component chromosome 1 surface marker-1, and tumor-associated calcium signal transducer-2) is a transmembrane glycoprotein that is upregulated in numerous cancer types, and is the protein product of the TACSTD2 gene. In some cases, a cancer-associated peptide is a peptide of a TROP-2 polypeptide having at least 90%, at least 95%, at least 98%, at least 99%, or 100%, amino acid sequence identity to the following TROP-2 amino acid sequence:

(SEQ ID NO: 1060)

QDNCTCPTNK MTVCSPDGPG GRCQCRALGS GMAVDCSTLT
SKCLLLKARM SAPKNARTLV RPSEHALVDN DGLYDPDCDP
EGRFKARQCN QTSVCWCVNS VGVRRTDKGD LSLRCDELVR
THHILIDLRH RPTAGAFNHS DLDAELRRLF RERYRLHPKF
VAAVHYEQPT IQIELRQNTS QKAAGDVDIG DAAYYFERDI
KGESLFQGRG GLDLRVRGEP LQVERTLIYY LDEIPPKFSM
KRLTAGLIAV IVVVVVALVA GMAVLVITNR RKSGKYKKVE
IKELGELRKE PSL.

### Antibodies

As noted above, in some cases, a TTP present in a TMMP is an antibody. In some cases, the TTP is an antibody that is specific for a cancer-associated antigen. In some cases, the TTP is an antibody specific for a peptide/HLA complex on the surface of a cancer cell, where the peptide can be a cancer-associated peptide (e.g., a peptide of a cancer-associated antigen).

Non-limiting examples of cancer-associated antigen-targeted antibodies that can be included in a TMMP include, but are not limited to, abituzumab (anti-CD51), LL1 (anti-CD74), LL2 or RFB4 (anti-CD22), veltuzumab (hA20, anti-CD20), binutuzu (anti-CD20), binutuzumab (GA101, anti-CD20), daratumumab (anti-CD38), lambrolizumab (anti-PD-1 receptor), nivolumab (anti-PD-1 receptor), ipilimumab (anti-CTLA-4), RS7 (anti-TROP-2), PAM4 or KC4 (both anti-mucin), MN-14 (anti-CEA), MN-15 or MN-3 (anti-CEACAM6), Mu-9 (anti-colon-specific antigen-p), Immu 31 (anti-alpha-fetoprotein), R1 (anti-IGF-1R), A19 (anti-CD19), TAG-72 (e.g., CC49), Tn, J591 or HuJ591 (anti-PSMA), AB-PG1-XG1-026 (anti-PSMA dimer), D2/B (anti-PSMA), G250 (anti-carbonic anhydrase IX), L243 (anti-HLA-DR) alemtuzumab (anti-CD52), oportuzumab (anti-EpCAM), bevacizumab (anti-VEGF), cetuximab (anti-EGFR), gemtuzumab (anti-CD33), ibritumomab tiuxetan (anti-CD20); panitumumab (anti-EGFR); tositumomab (anti-CD20); PAM4 (also known as clivatuzumab; anti-mucin), trastuzumab (anti-HER2), pertuzumab (anti-HER2), polatuzumab (anti-CD79b), and anetumab (anti-mesothelin).

In some cases, the tumor-targeting polypeptide is a single-chain antibody. In some cases, the tumor-targeting polypeptide is a scFv. In some cases, the tumor-targeting polypeptide is a nanobody (also referred to as a single domain antibody (sdAb)). In some cases, the tumor-targeting polypeptide is a heavy chain nanobody. In some cases, the tumor-targeting polypeptide is a light chain nanobody.

VH and VL amino acid sequences of various tumor antigen-binding antibodies are known in the art, as are the light chain and heavy chain CDRs of such antibodies. See, e.g., Ling et al. (2018) Frontiers Immunol. 9:469; WO 2005/012493; US 2019/0119375; US 2013/0066055. The following are non-limiting examples of tumor antigen-binding antibodies.

### Anti-Her2

In some cases, an anti-Her2 antibody comprises: a) a light chain comprising an amino acid sequence having at least 90%, at least 95%, at least 98%, at least 99%, or 100%, amino acid sequence identity to the following amino acid sequence:
DIQMTQSPSSLSASVGDRVTITCRASQDVNTAVAWYQQKPGKAPKLLIYSASFLY SGVPSRFSGSRSGTDFTLTISSLQPEDFATYYCQQHYTTPPTFGQGTKVEIKRTVAAPSVFIFPPSD EQLKSGTASVVCLLNNFYPREAKVQWKVDNALQSGNSQESVTEQDSKDSTYSLSSTLTLSKAD YEKHKVYACEVTHQGLSSPVTKSFNRGEC (SEQ ID NO:1061); and b) a heavy chain comprising an amino acid sequence having at least 90%, at least 95%, at least 98%, at least 99%, or 100%, amino acid sequence identity to the following amino acid sequence:

(SEQ ID NO: 1062)
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPG
K.

In some cases, an anti-Her2 antibody comprises a light chain variable region (VL) present in the light chain amino acid sequence provided above; and a heavy chain variable region (VH) present in the heavy chain amino acid sequence provided above. For example, an anti-Her2 antibody can comprise: a) a VL comprising an amino acid sequence having at least 90%, at least 95%, at least 98%, at least 99%, or 100%, amino acid sequence identity to the amino acid sequence: DIQMTQSPSSLSASVGDRVTITCRASQDVNTAVAWYQQKPGKAPKLLIYSASFLYSGVPSRFSGS RSGTDFTLTISSLQPEDFATYYCQQHYTTPPTFGQGTKVEIK (SEQ ID NO:1063); and b) a VH comprising an amino acid sequence having at least 90%, at least 95%, at least 98%, at least 99%, or 100%, amino acid sequence identity to the amino acid sequence: EVQLVESGGGLVQPGGSLRLSCAASGFNIKDTYIHWVRQAPGKGLEWVARIYPTNGYTRYADS VKGRFTISADTSKNTAYLQMNSLRAEDTAVYYCSRWGGDGFYAMDYWGQGTLVTVSS (SEQ ID NO:1064). In some cases, an anti-Her2 antibody comprises, in order from N-terminus to C-terminus: a) a VH comprising an amino acid sequence having at least 90%, at least 95%, at least 98%, at least 99%, or 100%, amino acid sequence identity to the amino acid sequence: EVQLVESGGGLVQPGGSLRLSCAASGFNIKDTYIHWVRQAPGKGLEWVARIYPTNGYTRYADS VKGRFTISADTSKNTAYLQMNSLRAEDTAVYYCSRWGGDGFYAMDYWGQGTLVTVSS (SEQ ID NO:1065); b) a linker; and c) a VL comprising an amino acid sequence having at least 90%, at least 95%, at least 98%, at least 99%, or 100%, amino acid sequence identity to the amino acid sequence: DIQMTQSPSSLSASVGDRVTITCRASQDVNTAVAWYQQKPGKAPKLLIYSASFLYSGVPSRFSGS RSGTDFTLTISSLQPEDFATYYCQQHYTTPPTFGQGTKVEIK (SEQ ID NO:1066). Suitable linkers are described elsewhere herein and include, e.g., (GGGGS)n (SEQ ID NO:934), where n is an integer from 1 to 10 (e.g., 1, 2, 3, 4, 5, 6, 7, 8, 9, or 10).

In some cases, an anti-Her2 antibody comprises VL CDR1, VL CDR2, and VL CDR3 present in the light chain amino acid sequence provided above; and VH CDR1, CDR2, and CDR3 present in the heavy chain amino acid sequence provided above. In some cases, the V_{H} and V_{L} CDRs are as defined by Kabat (see, e.g., Table 1, above; and Kabat 1991). In some cases, the V_{H} and V_{L} CDRs are as defined by Chothia (see, e.g., Table 1, above; and Chothia 1987).

For example, an anti-Her2 antibody can comprise a VL CDR1 having the amino acid sequence RASQDVNTAVA (SEQ ID NO:1067); a VL CDR2 having the amino acid sequence SASFLY (SEQ ID NO:1068); a VL CDR3 having the amino acid sequence QQHYTTPP (SEQ ID NO:1069); a VH CDR1 having the amino acid sequence GFNIKDTY (SEQ ID NO:1070); a VH CDR2 having the amino acid sequence IYPTNGYT (SEQ ID NO:1071); and a VH CDR3 having the amino acid sequence SRWGGDGFYAMDY (SEQ ID NO:1072).

In some cases, an anti-Her2 antibody is a scFv antibody. For example, an anti-Her2 scFv can comprise an amino acid sequence having at least 90%, at least 95%, at least 98%, at least 99%, or 100%, amino acid sequence identity to the following amino acid sequence:

(SEQ ID NO: 1073)
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSSGGGGSGGGGSGGGGSDIQMTQSPSSLSASV
GDRVTITCRASQDVNTAVAWYQQKPGKAPKLLIYSASFLYSGVPSRFSGS
RSGTDFTLTISSLQPEDFATYYCQQHYTTPPTFGQGTKVEIK.

As another example, in some cases, an anti-Her2 antibody comprises: a) a light chain variable region (VL) comprising an amino acid sequence having at least 90%, at least 95%, at least 98%, at least 99%, or 100%, amino acid sequence identity to the following amino acid sequence:
DIQMTQSPSSLSASVGDRVTITCKASQDVSIGVAWYQQKPGKAPKLLIYSASYRY TGVPSRFSGSGSGTDFTLTISSLQPEDFATYYCQQYYIYPYTFGQGTKVEIKRTVAAPSVFIFPPSD EQLKSGTASVVCLLNNFYPREAKVQWKVDNALQSGNSQESVTEQDSKDSTYSLSSTLTLSKAD YEKHKVYACEVTHQGLSSPVTKSFNRGEC (SEQ ID NO:1074); and b) a heavy chain variable region (VH) comprising an amino acid sequence having at least 90%, at least 95%, at least 98%, at least 99%, or 100%, amino acid sequence identity to the following amino acid sequence:

(SEQ ID NO: 1075)
EVOLVESGGGLVQPGGSLRLSCAASGFTFTDYTMDWVRQAPGKGLEWVAD
VNPNSGGSIYNQRFKGRFTLSVDRSKNTLYLQMNSLRAEDTAVYYCARNL
GPSFYFDYWGQGTLVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPG.

In some cases, an anti-Her2 antibody comprises a VL present in the light chain amino acid sequence provided above; and a VH present in the heavy chain amino acid sequence provided above. For example, an anti-Her2 antibody can comprise: a) a VL comprising an amino acid sequence having at least 90%, at least 95%, at least 98%, at least 99%, or 100%, amino acid sequence identity to the amino acid sequence: DIQMTQSPSSLSASVGDRVTITCKASQDVSIGVAWYQQKPGKAPKLLIYSASYRYTGVPSRFSGS GSGTDFTLTISSLQPEDFATYYCQQYYIYPYTFGQGTKVEIK (SEQ ID NO:1076); and b) a VH comprising an amino acid sequence having at least 90%, at least 95%, at least 98%, at least 99%, or 100%, amino acid sequence identity to the amino acid sequence:

(SEQ ID NO: 1077)
EVQLVESGGGLVQPGGSLRLSCAASGFTFTDYTMDWVRQAPGKGLEWVAD
VNPNSGGSIYNQRFKGRFTLSVDRSKNTLYLQMNSLRAEDTAVYYCARNL
GPSFYFDYWGQGTLVTVSS.

In some cases, an anti-Her2 antibody comprises VL CDR1, VL CDR2, and VL CDR3 present in the light chain amino acid sequence provided above; and VH CDR1, CDR2, and CDR3 present in the heavy chain amino acid sequence provided above. In some cases, the V_{H} and V_{L} CDRs are as defined by Kabat (see, e.g., Table 1, above; and Kabat 1991). In some cases, the V_{H} and V_{L} CDRs are as defined by Chothia (see, e.g., Table 1, above; and Chothia 1987).

For example, an anti-HER2 antibody can comprise a VL CDR1 having the amino acid sequence KASQDVSIGVA (SEQ ID NO:1078); a VL CDR2 having the amino acid sequence SASYRY (SEQ ID NO:1079); a VL CDR3 having the amino acid sequence QQYYIYPY (SEQ ID NO:1080); a VH CDR1 having the amino acid sequence GFTFTDYTMD (SEQ ID NO:1081); a VH CDR2 having the amino acid sequence ADVNPNSGGSIYNQRFKG (SEQ ID NO:1082); and a VH CDR3 having the amino acid sequence ARNLGPSFYFDY (SEQ ID NO:1083).

In some cases, an anti-Her2 antibody is a scFv. For example, in some cases, an anti-Her2 scFv comprises an amino acid sequence having at least 90%, at least 95%, at least 98%, at least 99%, or 100%, amino acid sequence identity to the following amino acid sequence:

(SEQ ID NO: 1073)
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSSGGGGSGGGGSGGGGSDIQMTQSPSSLSASV
GDRVTITCRASQDVNTAVAWYQQKPGKAPKLLIYSASFLYSGVPSRFSGS
RSGTDFTLTISSLQPEDFATYYCQQHYTTPPTFGQGTKVEIK.

### Anti-CD19

Anti-CD19 antibodies are known in the art; and the VH and VL, or the VH and VL CDRs, of any anti-CD19 antibody can be used in a TMMP. See e.g., WO 2005/012493.

In some cases, an anti-CD19 antibody includes a VL CDR1 comprising the amino acid sequence KASQSVDYDGDSYLN (SEQ ID NO:1084); a VL CDR2 comprising the amino acid sequence DASNLVS (SEQ ID NO:1085); and a VL CDR3 comprising the amino acid sequence QQSTEDPWT (SEQ ID NO:1086). In some cases, an anti-CD19 antibody includes a VH CDR1 comprising the amino acid sequence SYWMN (SEQ ID NO:1087); a VH CDR2 comprising the amino acid sequence QIWPGDGDTNYNGKFKG (SEQ ID NO:1088); and a VH CDR3 comprising the amino acid sequence RETTTVGRYYYAMDY (SEQ ID NO:1089). In some cases, an anti-CD19 antibody includes a VL CDR1 comprising the amino acid sequence KASQSVDYDGDSYLN (SEQ ID NO:1084); a VL CDR2 comprising the amino acid sequence DASNLVS (SEQ ID NO:1085); a VL CDR3 comprising the amino acid sequence QQSTEDPWT (SEQ ID NO:1086); a VH CDR1 comprising the amino acid sequence SYWMN (SEQ ID NO:1087); a VH CDR2 comprising the amino acid sequence QIWPGDGDTNYNGKFKG (SEQ ID NO:1088); and a VH CDR3 comprising the amino acid sequence RETTTVGRYYYAMDY (SEQ ID NO:1089).

In some cases, an anti-CD19 antibody is a scFv. For example, in some cases, an anti-CD19 scFv comprises an amino acid sequence having at least 90%, at least 95%, at least 98%, at least 99%, or 100%, amino acid sequence identity to the following amino acid sequence:

(SEQ ID NO: 1090)
DIQLTQSPASLAVSLGQRATISCKASQSVDYDGDSYLNWYQQIPGQPPKL
LIYDASNLVSGIPPRFSGSGSGTDFTLNIHPVEKVDAATYHCQQSTEDPW
TFGGGTKLEIKGGGGSGGGGSGGGGSQVQLQQSGAELVRPGSSVKISCKA
SGYAFSSYWMNWVKQRPGQGLEWIGQIWPGDGDTNYNGKFKGKATLTADE
SSSTAYMQLSSLASEDSAVYFCARRETTTVGRYYYAMDYWGQGTTVTVS.

### Anti-Mesothelin

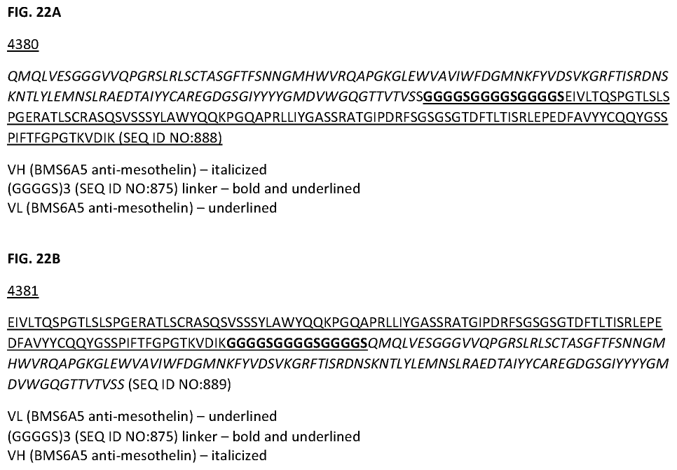

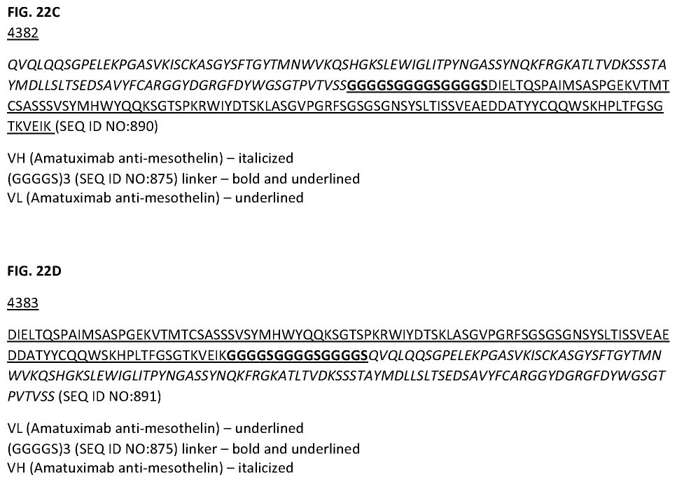

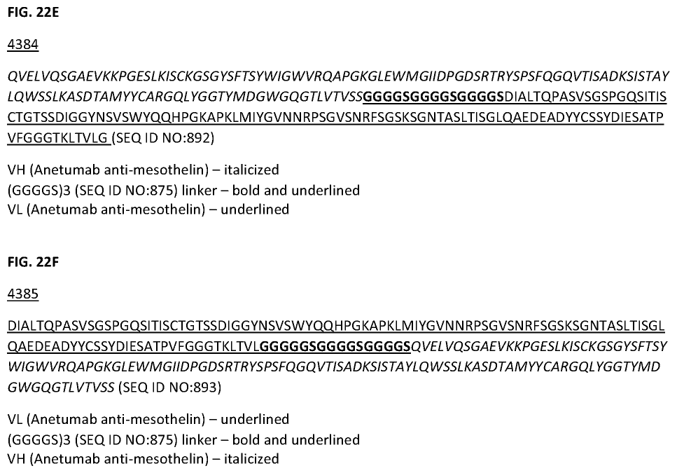

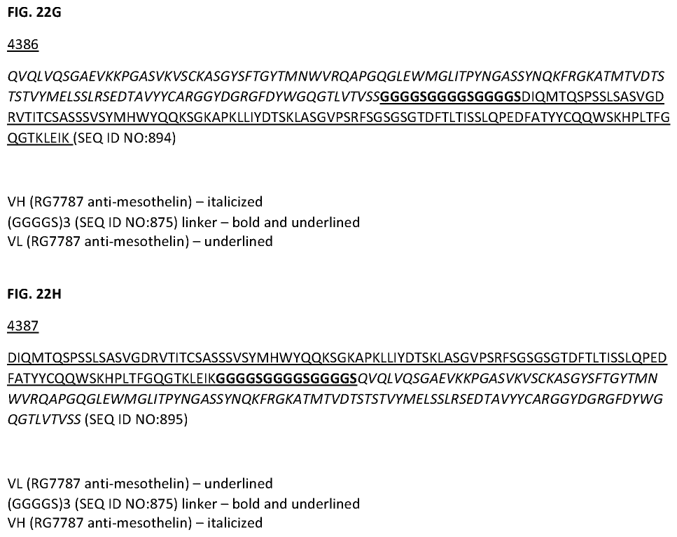

Anti-mesothelin antibodies are known in the art; and the VH and VL, or the VH and VL CDRs, of any anti-mesothelin antibody can be used in a TMMP. See, e.g., U.S. 2019/0000944; WO 2009/045957; WO 2014/031476; U.S. Pat. No. 8,460,660; US 2013/0066055; and WO 2009/068204. In some cases, the TTP is an anti-mesothelin scFv or an anti-mesothelin nanobody comprising VH and VL CDRs present in any one of the amino acid sequences set forth in FIG. 22A-22H. In some cases, the TTP is an anti-mesothelin scFv comprising an amino acid sequence as set forth in any one of FIG. 22A-22H.

In some cases, an anti-mesothelin antibody comprises: a) a light chain comprising an amino acid sequence having at least 90%, at least 95%, at least 98%, at least 99%, or 100%, amino acid sequence identity to the following amino acid sequence: DIALTQPASVSGSPGQSITISCTGTSSDIGGYNSVSWYQQHPGKAPKLMIYGVNNR PSGVSNRFSGSKSGNTASLTISGLQAEDEADYYCSSYDIESATPVFGGGTKLTVLGQPKAAPSVT LFPPSSEELQANKATLVCLISDFYPGAVTVAWKGDSSPVKAGVETTTPSKQSNNKYAASSYLSL TPEQWKSHRSYSCQVTHEGSTVEKTVAPTESS (SEQ ID NO:1091); and b) a heavy chain comprising an amino acid sequence having at least 90%, at least 95%, at least 98%, at least 99%, or 100%, amino acid sequence identity to the following amino acid sequence:

(SEQ ID NO: 1092)
QVELVQSGAEVKKPGESLKISCKGSGYSFTSYWIGWVRQAPGKGLEWMGI
IDPGDSRTRYSPSFQGQVTISADKSISTAYLQWSSLKASDTAMYYCARGQ
LYGGTYMDGWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPG
K.

In some cases, an anti-mesothelin antibody comprises a VL present in the light chain amino acid sequence provided above; and a VH present in the heavy chain amino acid sequence provided above. For example, an anti-mesothelin antibody can comprise: a) a VL comprising an amino acid sequence having at least 90%, at least 95%, at least 98%, at least 99%, or 100%, amino acid sequence identity to the amino acid sequence: DIALTQPASVSGSPGQSITISCTGTSSDIGGYNSVSWYQQHPGKAPKLMIYGVNNRPSGVSNRFS GSKSGNTASLTISGLQAEDEADYYCSSYDIESATPVFGGGTK (SEQ ID NO:1093); and b) a VH comprising an amino acid sequence having at least 90%, at least 95%, at least 98%, at least 99%, or 100%, amino acid sequence identity to the amino acid sequence:

(SEQ ID NO: 1094)
QVELVQSGAEVKKPGESLKISCKGSGYSFTSYWIGWVRQAPGKGLEWMGI
IDPGDSRTRYSPSFQGQVTISADKSISTAYLQWSSLKASDTAMYYCARGQ
LYGGTYMDGWGQGTLVTVSS.

In some cases, an anti-mesothelin antibody comprises VL CDR1, VL CDR2, and VL CDR3 present in the light chain amino acid sequence provided above; and VH CDR1, CDR2, and CDR3 present in the heavy chain amino acid sequence provided above. In some cases, the V_{H} and V_{L} CDRs are as defined by Kabat (see, e.g., Table 1, above; and Kabat 1991). In some cases, the V_{H} and V_{L} CDRs are as defined by Chothia (see, e.g., Table 1, above; and Chothia 1987).

For example, an anti-mesothelin antibody can comprise a VL CDR1 having the amino acid sequence TGTSSDIGGYNSVS (SEQ ID NO:1095); a VL CDR2 having the amino acid sequence LMIYGVNNRPS (SEQ ID NO:1096); a VL CDR3 having the amino acid sequence SSYDIESATP (SEQ ID NO:1097); a VH CDR1 having the amino acid sequence GYSFTSYWIG (SEQ ID NO:1098); a VH CDR2 having the amino acid sequence WMGIIDPGDSRTRYSP (SEQ ID NO:1099); and a VH CDR3 having the amino acid sequence GQLYGGTYMDG (SEQ ID NO:1100).

An anti-mesothelin antibody can be a scFv. As one non-limiting example, an anti-mesothelin scFv can comprise the following amino acid sequence:

(SEQ ID NO: 1101)
QVQLQQSGAEVKKPGASVKVSCKASGYTFTGYYMHWVRQAPGQGLEWMGR
INPNSGGTNYAQKFQGRVTMTRDTSISTAYMELSRLRSEDTAVYYCARGR
YYGMDVWGQGTMVTVSSGGGGSGGGGSGGGGSGGGGSEIVLTQSPATLSL
SPGERATISCRASQSVSSNFAWYQQRPGQAPRLLIYDASNRATGIPPRFS
GSGSGTDFTLTISSLEPEDFAAYYCHQRSNWLYTFGQGTKVDIK,

where VH CDR1, CDR2, and CDR3 are underlined; and VL CDR1, CDR2, and CDR3 are bolded and underlined.

As one non-limiting example, an anti-mesothelin scFv can comprise the following amino acid sequence:

(SEQ ID NO: 1102)
QVQLVQSGAEVKKPGASVKVSCKASGYTFTGYYMHWVRQAPGQGLEWMGW
INPNSGGTNYAQKFQGRVTMTRDTSISTAYMELSRLRSDDTAVYYCARDL
RRTVVTPRAYYGMDVWGQGTTVTVSSGGGGSGGGGSGGGGSGGGGSDIQL
TQSPSTLSASVGDRVTITCQASQDISNSLNWYQQKAGKAPKLLIYDASTL
ETGVPSRFSGSGSGTDFSFTISSLQPEDIATYYCQQHDNLPLTFGQGTKV
EIK,

where VH CDR1, CDR2, and CDR3 are underlined; and VL CDR1, CDR2, and CDR3 are bolded and underlined.

In some cases, an anti-mesothelin antibody suitable for inclusion in a TMMP comprises: a) VL CDR1, VL CDR2, and VL CDR3 present in a light chain variable region (VL) comprising the following amino acid sequence: EIVLTQSPGTLSLSPGERATLSCRASQSVSSSYLAWYQQKPGQAPRLLIYGASSRATGIPDRFSGS GSGTDFTLTISRLEPEDFAVYYCQQYGSSPIFTFGPGTKVDIK (SEQ ID NO:1103); and b) VH CDR1, CDR2, and CDR3 present in a heavy chain variable region (VH) comprising the following amino acid sequence: QMQLVESGGGVVQPGRSLRLSCTASGFTFSNNGMHWVRQAPGKGLEWVAVIWFDGMNKFYV DSVKGRFTISRDNSKNTLYLEMNSLRAEDTAIYYCAREGDGSGIYYYYGMDVWGQGTTVTVSS (SEQ ID NO:1104). In some cases, the V_{H} and V_{L} CDRs are as defined by Kabat (see, e.g., Table 1, above; and Kabat 1991). In some cases, the V_{H} and V_{L} CDRs are as defined by Chothia (see, e.g., Table 1, above; and Chothia 1987). See, e.g., BMS6A5.

In some cases, an anti-mesothelin antibody suitable for inclusion in a TMMP comprises: a) a VL region comprising an amino acid sequence having at least 90%, at least 95%, at least 98%, at least 99%, or 100%, amino acid sequence identity to the following amino acid sequence: EIVLTQSPGTLSLSPGERATLSCRASQSVSSSYLAWYQQKPGQAPRLLIYGASSRATGIPDRFSGS GSGTDFTLTISRLEPEDFAVYYCQQYGSSPIFTFGPGTKVDIK (SEQ ID NO:1103); and b) a VH region comprising an amino acid sequence having at least 90%, at least 95%, at least 98%, at least 99%, or 100%, amino acid sequence identity to the following amino acid sequence:

(SEQ ID NO: 1104)
QMQLVESGGGVVQPGRSLRLSCTASGFTFSNNGMHWVRQAPGKGLEWVAV
IWFDGMNKFYVDSVKGRFTISRDNSKNTLYLEMNSLRAEDTAIYYCAREG
DGSGIYYYYGMDVWGQGTTVTVSS

In some cases, an anti-mesothelin antibody suitable for inclusion in a TMMP is a scFv comprising, in order from N-terminus to C-terminus: a) a VH region comprising an amino acid sequence having at least 90%, at least 95%, at least 98%, at least 99%, or 100%, amino acid sequence identity to the following amino acid sequence: QMQLVESGGGVVQPGRSLRLSCTASGFTFSNNGMHWVRQAPGKGLEWVAVIWFDGMNKFYV DSVKGRFTISRDNSKNTLYLEMNSLRAEDTAIYYCAREGDGSGIYYYYGMDVWGQGTTVTVSS (SEQ ID NO:1104); b) a peptide linker; and c) a VL region comprising an amino acid sequence having at least 90%, at least 95%, at least 98%, at least 99%, or 100%, amino acid sequence identity to the following amino acid sequence: EIVLTQSPGTLSLSPGERATLSCRASQSVSSSYLAWYQQKPGQAPRLLIYGASSRATGIPDRFSGS GSGTDFTLTISRLEPEDFAVYYCQQYGSSPIFTFGPGTKVDIK (SEQ ID NO:1103). In some cases, the peptide linker comprises the amino acid sequence (GGGGS)n (SEQ ID NO:934), where n is an integer from 1 to 10 (e.g., where n is 1, 2, 3, 4, 5, 6, 7, 8, 9, or 10). In some cases, the peptide linker comprises the amino acid sequence GGGGSGGGGSGGGGS (SEQ ID NO:875) and has a length of 15 amino acids.

In some cases, an anti-mesothelin antibody suitable for inclusion in a TMMP is a scFv comprising, in order from N-terminus to C-terminus: a) a VL region comprising an amino acid sequence having at least 90%, at least 95%, at least 98%, at least 99%, or 100%, amino acid sequence identity to the following amino acid sequence: EIVLTQSPGTLSLSPGERATLSCRASQSVSSSYLAWYQQKPGQAPRLLIYGASSRATGIPDRFSGS GSGTDFTLTISRLEPEDFAVYYCQQYGSSPIFTFGPGTKVDIK (SEQ ID NO:1103); b) a peptide linker; and c) a VH region comprising an amino acid sequence having at least 90%, at least 95%, at least 98%, at least 99%, or 100%, amino acid sequence identity to the following amino acid sequence: QMQLVESGGGVVQPGRSLRLSCTASGFTFSNNGMHWVRQAPGKGLEWVAVIWFDGMNKFYV DSVKGRFTISRDNSKNTLYLEMNSLRAEDTAIYYCAREGDGSGIYYYYGMDVWGQGTTVTVSS (SEQ ID NO:1104). In some cases, the peptide linker comprises the amino acid sequence (GGGGS)n (SEQ ID NO:934), where n is an integer from 1 to 10 (e.g., where n is 1, 2, 3, 4, 5, 6, 7, 8, 9, or 10). In some cases, the peptide linker comprises the amino acid sequence GGGGSGGGGSGGGGS (SEQ ID NO:875) and has a length of 15 amino acids.

In some cases, an anti-mesothelin antibody suitable for inclusion in a TMMP comprises: a) VL CDR1, VL CDR2, and VL CDR3 present in a light chain variable region (VL) comprising the following amino acid sequence: DIELTQSPAIMSASPGEKVTMTCSASSSVSYMHWYQQKSGTSPKRWIYDTSKLASGVPGRFSGS GSGNSYSLTISSVEAEDDATYYCQQWSKHPLTFGSGTKVEIK (SEQ ID NO:1105); and b) VH CDR1, CDR2, and CDR3 present in a heavy chain variable region (VH) comprising the following amino acid sequence: QVQLQQSGPELEKPGASVKISCKASGYSFTGYTMNWVKQSHGKSLEWIGLITPYNGASSYNQKF RGKATLTVDKSSSTAYMDLLSLTSEDSAVYFCARGGYDGRGFDYWGSGTPVTVSS (SEQ ID NO:1106). In some cases, the V_{H} and V_{L} CDRs are as defined by Kabat (see, e.g., Table 1, above; and Kabat 1991). In some cases, the V_{H} and V_{L} CDRs are as defined by Chothia (see, e.g., Table 1, above; and Chothia 1987). See, e.g., Amatuximab.

In some cases, an anti-mesothelin antibody suitable for inclusion in a TMMP comprises: a) a VL region comprising an amino acid sequence having at least 90%, at least 95%, at least 98%, at least 99%, or 100%, amino acid sequence identity to the following amino acid sequence: DIELTQSPAIMSASPGEKVTMTCSASSSVSYMHWYQQKSGTSPKRWIYDTSKLASGVPGRFSGS GSGNSYSLTISSVEAEDDATYYCQQWSKHPLTFGSGTKVEIK (SEQ ID NO:1105); and b) a VH region comprising an amino acid sequence having at least 90%, at least 95%, at least 98%, at least 99%, or 100%, amino acid sequence identity to the following amino acid sequence:

(SEQ ID NO: 1106)
QVQLQQSGPELEKPGASVKISCKASGYSFTGYTMNWVKQSHGKSLEWIGL
ITPYNGASSYNQKFRGKATLTVDKSSSTAYMDLLSLTSEDSAVYFCARGG
YDGRGFDYWGSGTPVTVSS.

In some cases, an anti-mesothelin antibody suitable for inclusion in a TMMP is a scFv comprising, in order from N-terminus to C-terminus: a) a VL region comprising an amino acid sequence having at least 90%, at least 95%, at least 98%, at least 99%, or 100%, amino acid sequence identity to the following amino acid sequence: DIELTQSPAIMSASPGEKVTMTCSASSSVSYMHWYQQKSGTSPKRWIYDTSKLASGVPGRFSGS GSGNSYSLTISSVEAEDDATYYCQQWSKHPLTFGSGTKVEIK (SEQ ID NO:1105); b) a peptide linker; and c) a VH region comprising an amino acid sequence having at least 90%, at least 95%, at least 98%, at least 99%, or 100%, amino acid sequence identity to the following amino acid sequence: QVQLQQSGPELEKPGASVKISCKASGYSFTGYTMNWVKQSHGKSLEWIGLITPYNGASSYNQKF RGKATLTVDKSSSTAYMDLLSLTSEDSAVYFCARGGYDGRGFDYWGSGTPVTVSS (SEQ ID NO:1106). In some cases, the peptide linker comprises the amino acid sequence (GGGGS)n (SEQ ID NO:934), where n is an integer from 1 to 10 (e.g., where n is 1, 2, 3, 4, 5, 6, 7, 8, 9, or 10). In some cases, the peptide linker comprises the amino acid sequence GGGGSGGGGSGGGGS (SEQ ID NO:875) and has a length of 15 amino acids.

In some cases, an anti-mesothelin antibody suitable for inclusion in a TMMP is a scFv comprising, in order from N-terminus to C-terminus: a) a VH region comprising an amino acid sequence having at least 90%, at least 95%, at least 98%, at least 99%, or 100%, amino acid sequence identity to the following amino acid sequence: QVQLQQSGPELEKPGASVKISCKASGYSFTGYTMNWVKQSHGKSLEWIGLITPYNGASSYNQKF RGKATLTVDKSSSTAYMDLLSLTSEDSAVYFCARGGYDGRGFDYWGSGTPVTVSS (SEQ ID NO:1106); b) a peptide linker; and c) a VL region comprising an amino acid sequence having at least 90%, at least 95%, at least 98%, at least 99%, or 100%, amino acid sequence identity to the following amino acid sequence: DIELTQSPAIMSASPGEKVTMTCSASSSVSYMHWYQQKSGTSPKRWIYDTSKLASGVPGRFSGS GSGNSYSLTISSVEAEDDATYYCQQWSKHPLTFGSGTKVEIK (SEQ ID NO:1105). In some cases, the peptide linker comprises the amino acid sequence (GGGGS)n (SEQ ID NO:934), where n is an integer from 1 to 10 (e.g., where n is 1, 2, 3, 4, 5, 6, 7, 8, 9, or 10). In some cases, the peptide linker comprises the amino acid sequence GGGGSGGGGSGGGGS (SEQ ID NO:875) and has a length of 15 amino acids.

In some cases, an anti-mesothelin antibody suitable for inclusion in a TMMP comprises: a) VL CDR1, VL CDR2, and VL CDR3 present in a light chain variable region (VL) comprising the following amino acid sequence: DIALTQPASVSGSPGQSITISCTGTSSDIGGYNSVSWYQQHPGKAPKLMIYGVNNRPSGVSNRFS GSKSGNTASLTISGLQAEDEADYYCSSYDIESATPVFGGGTKLTVLG (SEQ ID NO:1107); and b) VH CDR1, CDR2, and CDR3 present in a heavy chain variable region (VH) comprising the following amino acid sequence: QVELVQSGAEVKKPGESLKISCKGSGYSFTSYWIGWVRQAPGKGLEWMGIIDPGDSRTRYSPSF QGQVTISADKSISTAYLQWSSLKASDTAMYYCARGQLYGGTYMDGWGQGTLVTVSS (SEQ ID NO:1094). In some cases, the V_{H} and V_{L} CDRs are as defined by Kabat (see, e.g., Table 1, above; and Kabat 1991). In some cases, the V_{H} and V_{L} CDRs are as defined by Chothia (see, e.g., Table 1, above; and Chothia 1987).

In some cases, an anti-mesothelin antibody suitable for inclusion in a TMMP comprises: a) a VL region comprising an amino acid sequence having at least 90%, at least 95%, at least 98%, at least 99%, or 100%, amino acid sequence identity to the following amino acid sequence: DIALTQPASVSGSPGQSITISCTGTSSDIGGYNSVSWYQQHPGKAPKLMIYGVNNRPSGVSNRFS GSKSGNTASLTISGLQAEDEADYYCSSYDIESATPVFGGGTKLTVLG (SEQ ID NO:1107); and b) a VH region comprising an amino acid sequence having at least 90%, at least 95%, at least 98%, at least 99%, or 100%, amino acid sequence identity to the following amino acid sequence:

(SEQ ID NO: 1094)
QVELVQSGAEVKKPGESLKISCKGSGYSFTSYWIGWVRQAPGKGLEWMGI
IDPGDSRTRYSPSFQGQVTISADKSISTAYLQWSSLKASDTAMYYCARGQ
LYGGTYMDGWGQGTLVTVSS.

In some cases, an anti-mesothelin antibody suitable for inclusion in a TMMP is a scFv comprising, in order from N-terminus to C-terminus: a) a VL region comprising an amino acid sequence having at least 90%, at least 95%, at least 98%, at least 99%, or 100%, amino acid sequence identity to the following amino acid sequence: DIALTQPASVSGSPGQSITISCTGTSSDIGGYNSVSWYQQHPGKAPKLMIYGVNNRPSGVSNRFS GSKSGNTASLTISGLQAEDEADYYCSSYDIESATPVFGGGTKLTVLG (SEQ ID NO:1107); b) a peptide linker; and c) a VH region comprising an amino acid sequence having at least 90%, at least 95%, at least 98%, at least 99%, or 100%, amino acid sequence identity to the following amino acid sequence: QVELVQSGAEVKKPGESLKISCKGSGYSFTSYWIGWVRQAPGKGLEWMGIIDPGDSRTRYSPSF QGQVTISADKSISTAYLQWSSLKASDTAMYYCARGQLYGGTYMDGWGQGTLVTVSS (SEQ ID NO:1094). In some cases, the peptide linker comprises the amino acid sequence (GGGGS)n (SEQ ID NO:934), where n is an integer from 1 to 10 (e.g., where n is 1, 2, 3, 4, 5, 6, 7, 8, 9, or 10). In some cases, the peptide linker comprises the amino acid sequence GGGGSGGGGSGGGGS (SEQ ID NO:875) and has a length of 15 amino acids.

In some cases, an anti-mesothelin antibody suitable for inclusion in a TMMP is a scFv comprising, in order from N-terminus to C-terminus: a) a VH region comprising an amino acid sequence having at least 90%, at least 95%, at least 98%, at least 99%, or 100%, amino acid sequence identity to the following amino acid sequence: QVELVQSGAEVKKPGESLKISCKGSGYSFTSYWIGWVRQAPGKGLEWMGIIDPGDSRTRYSPSF QGQVTISADKSISTAYLQWSSLKASDTAMYYCARGQLYGGTYMDGWGQGTLVTVSS (SEQ ID NO:1094); b) a peptide linker; and c) a VL region comprising an amino acid sequence having at least 90%, at least 95%, at least 98%, at least 99%, or 100%, amino acid sequence identity to the following amino acid sequence: DIALTQPASVSGSPGQSITISCTGTSSDIGGYNSVSWYQQHPGKAPKLMIYGVNNRPSGVSNRFS GSKSGNTASLTISGLQAEDEADYYCSSYDIESATPVFGGGTKLTVLG (SEQ ID NO:1107). In some cases, the peptide linker comprises the amino acid sequence (GGGGS)n (SEQ ID NO:934), where n is an integer from 1 to 10 (e.g., where n is 1, 2, 3, 4, 5, 6, 7, 8, 9, or 10). In some cases, the peptide linker comprises the amino acid sequence GGGGSGGGGSGGGGS (SEQ ID NO:875) and has a length of 15 amino acids.

In some cases, an anti-mesothelin antibody suitable for inclusion in a TMMP comprises: a) VL CDR1, VL CDR2, and VL CDR3 present in a light chain variable region (VL) comprising the following amino acid sequence: DIQMTQSPSSLSASVGDRVTITCSASSSVSYMHWYQQKSGKAPKLLIYDTSKLASGVPSRFSGSG SGTDFTLTISSLQPEDFATYYCQQWSKHPLTFGQGTKLEIK (SEQ ID NO:1108); and b) VH CDR1, CDR2, and CDR3 present in a heavy chain variable region (VH) comprising the following amino acid sequence: QVQLVQSGAEVKKPGASVKVSCKASGYSFTGYTMNWVRQAPGQGLEWMGLITPYNGASSYN QKFRGKATMTVDTSTSTVYMELSSLRSEDTAVYYCARGGYDGRGFDYWGQGTLVTVSS (SEQ ID NO:1109). In some cases, the V_{H} and V_{L} CDRs are as defined by Kabat (see, e.g., Table 1, above; and Kabat 1991). In some cases, the V_{H} and V_{L} CDRs are as defined by Chothia (see, e.g., Table 1, above; and Chothia 1987). See, e.g., RG7787.

In some cases, an anti-mesothelin antibody suitable for inclusion in a TMMP comprises: a) a VL region comprising an amino acid sequence having at least 90%, at least 95%, at least 98%, at least 99%, or 100%, amino acid sequence identity to the following amino acid sequence: DIQMTQSPSSLSASVGDRVTITCSASSSVSYMHWYQQKSGKAPKLLIYDTSKLASGVPSRFSGSG SGTDFTLTISSLQPEDFATYYCQQWSKHPLTFGQGTKLEIK (SEQ ID NO:1108); and b) a VH region comprising an amino acid sequence having at least 90%, at least 95%, at least 98%, at least 99%, or 100%, amino acid sequence identity to the following amino acid sequence:

(SEQ ID NO: 1109)
QVQLVQSGAEVKKPGASVKVSCKASGYSFTGYTMNWVRQAPGQGLEWMGL
ITPYNGASSYNQKFRGKATMTVDTSTSTVYMELSSLRSEDTAVYYCARGG
YDGRGFDYWGQGTLVTVSS.

In some cases, an anti-mesothelin antibody suitable for inclusion in a TMMP is a scFv comprising, in order from N-terminus to C-terminus: a) a VL region comprising an amino acid sequence having at least 90%, at least 95%, at least 98%, at least 99%, or 100%, amino acid sequence identity to the following amino acid sequence: DIQMTQSPSSLSASVGDRVTITCSASSSVSYMHWYQQKSGKAPKLLIYDTSKLASGVPSRFSGSG SGTDFTLTISSLQPEDFATYYCQQWSKHPLTFGQGTKLEIK (SEQ ID NO:1108); b) a peptide linker; and c) a VH region comprising an amino acid sequence having at least 90%, at least 95%, at least 98%, at least 99%, or 100%, amino acid sequence identity to the following amino acid sequence: QVQLVQSGAEVKKPGASVKVSCKASGYSFTGYTMNWVRQAPGQGLEWMGLITPYNGASSYN QKFRGKATMTVDTSTSTVYMELSSLRSEDTAVYYCARGGYDGRGFDYWGQGTLVTVSS (SEQ ID NO:1109). In some cases, the peptide linker comprises the amino acid sequence (GGGGS)n (SEQ ID NO:934), where n is an integer from 1 to 10 (e.g., where n is 1, 2, 3, 4, 5, 6, 7, 8, 9, or 10). In some cases, the peptide linker comprises the amino acid sequence GGGGSGGGGSGGGGS (SEQ ID NO:875) and has a length of 15 amino acids.

In some cases, an anti-mesothelin antibody suitable for inclusion in a TMMP is a scFv comprising, in order from N-terminus to C-terminus: a) a VH region comprising an amino acid sequence having at least 90%, at least 95%, at least 98%, at least 99%, or 100%, amino acid sequence identity to the following amino acid sequence: QVQLVQSGAEVKKPGASVKVSCKASGYSFTGYTMNWVRQAPGQGLEWMGLITPYNGASSYN QKFRGKATMTVDTSTSTVYMELSSLRSEDTAVYYCARGGYDGRGFDYWGQGTLVTVSS (SEQ ID NO:1109); b) a peptide linker; and c) a VL region comprising an amino acid sequence having at least 90%, at least 95%, at least 98%, at least 99%, or 100%, amino acid sequence identity to the following amino acid sequence: DIQMTQSPSSLSASVGDRVTITCSASSSVSYMHWYQQKSGKAPKLLIYDTSKLASGVPSRFSGSG SGTDFTLTISSLQPEDFATYYCQQWSKHPLTFGQGTKLEIK (SEQ ID NO:1108). In some cases, the peptide linker comprises the amino acid sequence (GGGGS)n (SEQ ID NO:934), where n is an integer from 1 to 10 (e.g., where n is 1, 2, 3, 4, 5, 6, 7, 8, 9, or 10). In some cases, the peptide linker comprises the amino acid sequence GGGGSGGGGSGGGGS (SEQ ID NO:875) and has a length of 15 amino acids.

In some cases, an anti-mesothelin scFv comprises the following amino acid sequence:

(SEQ ID NO: 888)
QMQLVESGGGVVQPGRSLRLSCTASGFTFSNNGMHWVRQAPGKGLEWVAV
IWFDGMNKFYVDSVKGRFTISRDNSKNTLYLEMNSLRAEDTAIYYCAREG
DGSGIYYYYGMDVWGQGTTVTVSSGGGGSGGGGSGGGGSEIVLTQSPGTL
SLSPGERATLSCRASQSVSSSYLAWYQQKPGQAPRLLIYGASSRATGIPD
RESGSGSGTDFTLTISRLEPEDFAVYYCQQYGSSPIFTFGPGTKVDIK,

the VH sequence is italicized, the (GGGGS)3 (SEQ ID NO:875) linker is bolded and underlined, and the VL sequence is underlined.

In some cases, an anti-mesothelin scFv comprises the following amino acid sequence:

(SEQ ID NO: 889)
EIVLTQSPGTLSLSPGERATLSCRASQSVSSSYLAWYQQKPGQAPRLLIY
GASSRATGIPDRESGSGSGTDFTLTISRLEPEDFAVYYCQQYGSSPIFTF
GPGTKVDIKGGGGSGGGGSGGGGSQMQLVESGGGVVQPGRSLRLSCTASG
FTFSNNGMHWVRQAPGKGLEWVAVIWFDGMNKFYVDSVKGRFTISRDNSK
NTLYLEMNSLRAEDTAIYYCAREGDGSGIYYYYGMDVWGQGTTVTVSS,

where the VL sequence is underlined, the (GGGGS)3 (SEQ ID NO:875) linker is bolded and underlined, and the VL sequence is italicized.

In some cases, an anti-mesothelin scFv comprises the following amino acid sequence:

(SEQ ID NO: 890)
QVQLQQSGPELEKPGASVKISCKASGYSFTGYTMNWVKQSHGKSLEWIGL
ITPYNGASSYNQKFRGKATLTVDKSSSTAYMDLLSLTSEDSAVYFCARGG
YDGRGFDYWGSGTPVTVSSGGGGSGGGGSGGGGSDIELTOSPAIMSASPG
EKVTMTCSASSSVSYMHWYQQKSGTSPKRWIYDTSKLASGVPGRESGSGS
GNSYSLTISSVEAEDDATYYCQQWSKHPLTFGSGTKVEIK,

where the VH sequence is italicized, the (GGGGS)3 (SEQ ID NO:875) linker is bolded and underlined, and the VL sequence is underlined.

In some cases, an anti-mesothelin scFv comprises the following amino acid sequence:

(SEQ ID NO: 891)
DIELTQSPAIMSASPGEKVTMTCSASSSVSYMHWYQQKSGTSPKRWIYDTS
KLASGVPGRESGSGSGNSYSLTISSVEAEDDATYYCQQWSKHPLTFGSGTK
VEIKGGGGSGGGGSGGGGSQVQLQQSGPELEKPGASVKISCKASGYSFTGY
TMNWVKQSHGKSLEWIGLITPYNGASSYNQKFRGKATLTVDKSSSTAYMDL
LSLTSEDSAVYFCARGGYDGRGFDYWGSGTPVTVSS,

where the V L sequence is underlined, the (GGGGS)3 (SEQ ID NO:875) linker is bolded and underlined, and the VL sequence is italicized.

In some cases, an anti-mesothelin scFv comprises the following amino acid sequence:

(SEQ ID NO: 892)
QVELVQSGAEVKKPGESLKISCKGSGYSFTSYWIGWVRQAPGKGLEWMGII
DPGDSRTRYSPSFQGQVTISADKSISTAYLQWSSLKASDTAMYYCARGQLY
GGTYMDGWGQGTLVTVSSGGGGSGGGGSGGGGSDIALTQPASVSGSPGQSI
TISCTGTSSDIGGYNSVSWYQQHPGKAPKLMIYGVNNRPSGVSNRFSGSKS
GNTASLTISGLQAEDEADYYCSSYDIESATPVFGGGTKLTVLG,

where the VH sequence is italicized, the (GGGGS)3 (SEQ ID NO:875) linker is bolded and underlined, and the VL sequence is underlined.

In some cases, an anti-mesothelin scFv comprises the following amino acid sequence:

(SEQ ID NO: 893)
DIALTQPASVSGSPGQSITISCTGTSSDIGGYNSVSWYQQHPGKAPKLMIY
GVNNRPSGVSNRFSGSKSGNTASLTISGLQAEDEADYYCSSYDIESATPVF
GGGTKLTVLGGGGGSGGGGSGGGGSQVELVQSGAEVKKPGESLKISCKGSG
YSFTSYWIGWVRQAPGKGLEWMGIIDPGDSRTRYSPSFQGQVTISADKSIS
TAYLQWSSLKASDTAMYYCARGQLYGGTYMDGWGQGTLVTVSS,

where the VL sequence is underlined, the (GGGGS)3 (SEQ ID NO:875) linker is bolded and underlined, and the VL sequence is italicized.

In some cases, an anti-mesothelin scFv comprises the following amino acid sequence:

(SEQ ID NO:894)
QVQLVQSGAEVKKPGASVKVSCKASGYSFTGYTMNWVRQAPGQGLEWMGLI
TPYNGASSYNQKFRGKATMTVDTSTSTVYMELSSLRSEDTAVYYCARGGYD
GRGFDYWGQGTLVTVSSGGGGSGGGGSGGGGSDIQMTQSPSSLSASVGDRV
TITCSASSSVSYMHWYQQKSGKAPKLLIYDTSKLASGVPSRESGSGSGTDF
TLTISSLQPEDFATYYCQQWSKHPLTFGQGTKLEIK,

where the VH sequence is italicized, the (GGGGS)3 (SEQ ID NO:875) linker is bolded and underlined, and the VL sequence is underlined.

In some cases, an anti-mesothelin scFv comprises the following amino acid sequence:

(SEQ ID NO: 895)
DIQMTQSPSSLSASVGDRVTITCSASSSVSYMHWYQQKSGKAPKLLIYDTS
KLASGVPSRFSGSGSGTDFTLTISSLQPEDFATYYCQQWSKHPLTFGQGTK
LEIKGGGGSGGGGSGGGGSQVQLVQSGAEVKKPGASVKVSCKASGYSFTGY
TMNWVRQAPGQGLEWMGLITPYNGASSYNQKFRGKATMTVDTSTSTVYMEL
SSLRSEDTAVYYCARGGYDGRGFDYWGQGTLVTVSS,

where the VL sequence is underlined, the (GGGGS)3 (SEQ ID NO:875) linker is bolded and underlined, and the VL sequence is italicized.

### Anti-PSMA

Prostate-specific membrane antigen (PSMA) (also known as folate hydrolase 1 (FOLH1); membrane glutamate carboxypeptidase, and N-Acetylated-Alpha-Linked Acidic Dipeptidase 1) that is up-regulated in cancerous cells in the prostate and is used as a diagnostic and prognostic indicator of prostate cancer.

Anti-PSMA antibodies are known in the art; and the VH and VL, or the VH and VL CDRs, of any anti-PSMA antibody can be used in a TMMP. See, e.g., U.S. Pat. No. 10,179,819 and U.S. Patent Publication No. 2021/0277141.

### Anti-CD22

CD22 (also known as B-Lymphocyte Cell Adhesion Molecule, Sialic Acid-Binding Ig-Like Lectin 2, or SIGLEC2) is a sialic acid-binding adhesion molecule largely restricted to the B cell lineage and expressed on most B-lineage malignancies.

Anti-CD22 antibodies are known in the art; and the VH and VL, or the VH and VL CDRs, of any anti-CD22 antibody can be used in a TMMP. See, e.g., Xiao et al. (2009) Mabs 1:297 (describing the fully human anti-CD22 m971 scFv); and U.S. Patent Publication No. 2020/0147134. Examples of anti-CD22 antibodies include epratuzumab and inotuzumab. See, e.g., Lenoard et al. (2007) Oncogene 26:3704 and U.S. Pat. No. 5,789,554 (describing epratuzumab); and DiJoseph et al. (2007) Leukemia 21:2240 (describing inotuzumab).

For example, an anti-CD22 antibody can comprise: i) a heavy chain variable region (VH) CDR1 having the amino acid sequence: GDSVSSNSAA (SEQ ID NO:1110); ii) a VH CDR2 having the amino acid sequence: TYYRSKWYN (SEQ ID NO:1111); iii) a VH CDR3 having the amino acid sequence: AREVTGDLEDAFDI (SEQ ID NO:1112); iv) a light chain variable region (VL) CDR1 having the amino acid sequence: QTIWSY (SEQ ID NO:1113); v) a VL CDR2 having the amino acid sequence: AAS (Ala-Ala-Ser); and vi) a VL CDR3 having the amino acid sequence: QQSYSIPQT (SEQ ID NO:1114).

### Anti-TROP-2

Trophoblast cell surface antigen 2 (Trop-2) (also known as epithelial glycoprotein-1, gastrointestinal tumor-associated antigen GA733-1, membrane component chromosome 1 surface marker-1, and tumor-associated calcium signal transducer-2) is a transmembrane glycoprotein that is upregulated in numerous cancer types, and is the protein product of the TACSTD2 gene.

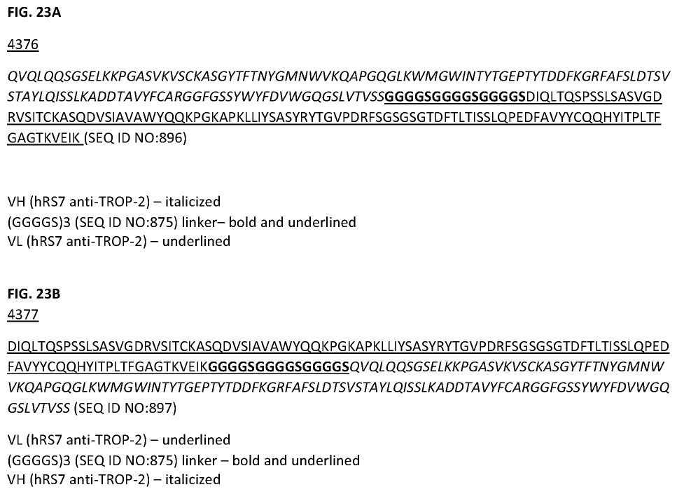

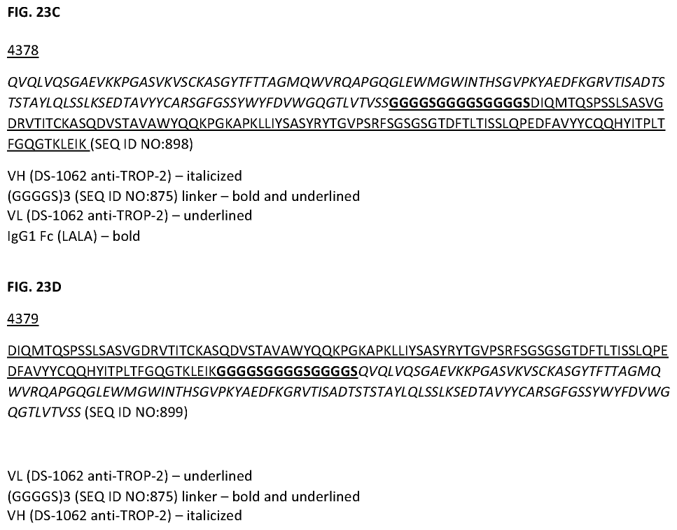

In some cases, the TTP is an anti-TROP-2 scFv or an anti-TROP-2 nanobody comprising VH and VL CDRs present in any one of the amino acid sequences set forth in FIG. 23A-23D. In some cases, the TTP is an anti-TROP-2 scFv comprising an amino acid sequence as set forth in any one of FIG. 23A-23D.

Anti-TROP-2 antibodies are known in the art; and the VH and VL, or the VH and VL CDRs, of any anti-TROP-2 antibody can be used in a TMMP. See, e.g., U.S. Pat. No. 7,238,785). In some cases, an anti-TROP-2 antibody comprises: i) light chain CDR sequences CDR1 (KASQDVSIAVA; SEQ ID NO:1115); CDR2 (SASYRYT; SEQ ID NO:1116); and CDR3 (QQHYITPLT; SEQ ID NO:1117); and ii) heavy chain CDR sequences CDR1 (NYGMN; SEQ ID NO:1118); CDR2 (WINTYTGEPTYTDDFKG; SEQ ID NO:1119); and CDR3 (GGFGSSYWYFDV; SEQ ID NO:1120).

In some cases, an anti-TROP-2 antibody comprises: i) heavy chain CDR sequences CDR1 (TAGMQ; SEQ ID NO:1121); CDR2 (WINTHSGVPKYAEDFKG (SEQ ID NO:1122); and CDR3 (SGFGSSYWYFDV; SEQ ID NO:1123); and ii) light chain CDR sequences CDR1 (KASQDVSTAVA; SEQ ID NO:1124); CDR2 (SASYRYT; SEQ ID NO:1116); and CDR3 (QQHYITPLT; SEQ ID NO:1117).

In some cases, an anti-TROP2 antibody suitable for inclusion in a TMMP comprises: a) VL CDR1, VL CDR2, and VL CDR3 present in a light chain variable region (VL) comprising the following amino acid sequence: DIQLTQSPSSLSASVGDRVSITCKASQDVSIAVAWYQQKPGKAPKLLIYSASYRYTGVPDRFSGS GSGTDFTLTISSLQPEDFAVYYCQQHYITPLTFGAGTKVEIK (SEQ ID NO:1125); and b) VH CDR1, CDR2, and CDR3 present in a heavy chain variable region (VH) comprising the following amino acid sequence: QVQLQQSGSELKKPGASVKVSCKASGYTFTNYGMNWVKQAPGQGLKWMGWINTYTGEPTYT DDFKGRFAFSLDTSVSTAYLQISSLKADDTAVYFCARGGFGSSYWYFDVWGQGSLVTVSS (SEQ ID NO:1126). In some cases, the V_{H} and V_{L} CDRs are as defined by Kabat (see, e.g., Table 1, above; and Kabat 1991). In some cases, the V_{H} and V_{L} CDRs are as defined by Chothia (see, e.g., Table 1, above; and Chothia 1987).

In some cases, an anti-TROP-2 antibody suitable for inclusion in a TMMP comprises: a) a VL region comprising an amino acid sequence having at least 90%, at least 95%, at least 98%, at least 99%, or 100%, amino acid sequence identity to the following amino acid sequence: DIQLTQSPSSLSASVGDRVSITCKASQDVSIAVAWYQQKPGKAPKLLIYSASYRYTGVPDRFSGS GSGTDFTLTISSLQPEDFAVYYCQQHYITPLTFGAGTKVEIK (SEQ ID NO:1125); and b) a VH region comprising an amino acid sequence having at least 90%, at least 95%, at least 98%, at least 99%, or 100%, amino acid sequence identity to the following amino acid sequence:

(SEQ ID NO: 1126)
QVQLQQSGSELKKPGASVKVSCKASGYTFTNYGMNWVKQAPGQGLKWMGWI
NTYTGEPTYTDDFKGRFAFSLDTSVSTAYLQISSLKADDTAVYFCARGGFG
SSYWYFDVWGQGSLVTVSS.

In some cases, an anti-TROP-2 antibody suitable for inclusion in a TMMP is a scFv comprising, in order from N-terminus to C-terminus: a) a VL region comprising an amino acid sequence having at least 90%, at least 95%, at least 98%, at least 99%, or 100%, amino acid sequence identity to the following amino acid sequence: DIQLTQSPSSLSASVGDRVSITCKASQDVSIAVAWYQQKPGKAPKLLIYSASYRYTGVPDRFSGS GSGTDFTLTISSLQPEDFAVYYCQQHYITPLTFGAGTKVEIK (SEQ ID NO:1125); b) a peptide linker; and c) a VH region comprising an amino acid sequence having at least 90%, at least 95%, at least 98%, at least 99%, or 100%, amino acid sequence identity to the following amino acid sequence: QVQLQQSGSELKKPGASVKVSCKASGYTFTNYGMNWVKQAPGQGLKWMGWINTYTGEPTYT DDFKGRFAFSLDTSVSTAYLQISSLKADDTAVYFCARGGFGSSYWYFDVWGQGSLVTVSS (SEQ ID NO:1126). In some cases, the peptide linker comprises the amino acid sequence (GGGGS)n (SEQ ID NO:934), where n is an integer from 1 to 10 (e.g., where n is 1, 2, 3, 4, 5, 6, 7, 8, 9, or 10). In some cases, the peptide linker comprises the amino acid sequence GGGGSGGGGSGGGGS (SEQ ID NO:875) and has a length of 15 amino acids.

In some cases, an anti-TROP-2 antibody suitable for inclusion in a TMMP is a scFv comprising, in order from N-terminus to C-terminus: a) a VH region comprising an amino acid sequence having at least 90%, at least 95%, at least 98%, at least 99%, or 100%, amino acid sequence identity to the following amino acid sequence: QVQLQQSGSELKKPGASVKVSCKASGYTFTNYGMNWVKQAPGQGLKWMGWINTYTGEPTYT DDFKGRFAFSLDTSVSTAYLQISSLKADDTAVYFCARGGFGSSYWYFDVWGQGSLVTVSS (SEQ ID NO:1126); b) a peptide linker; and c) a VL region comprising an amino acid sequence having at least 90%, at least 95%, at least 98%, at least 99%, or 100%, amino acid sequence identity to the following amino acid sequence: DIQLTQSPSSLSASVGDRVSITCKASQDVSIAVAWYQQKPGKAPKLLIYSASYRYTGVPDRFSGS GSGTDFTLTISSLQPEDFAVYYCQQHYITPLTFGAGTKVEIK (SEQ ID NO:1125). In some cases, the peptide linker comprises the amino acid sequence (GGGGS)n (SEQ ID NO:934), where n is an integer from 1 to 10 (e.g., where n is 1, 2, 3, 4, 5, 6, 7, 8, 9, or 10). In some cases, the peptide linker comprises the amino acid sequence GGGGSGGGGSGGGGS (SEQ ID NO:875) and has a length of 15 amino acids.

In some cases, an anti-TROP2 antibody suitable for inclusion in a TMMP comprises: a) VL CDR1, VL CDR2, and VL CDR3 present in a light chain variable region (VL) comprising the following amino acid sequence: DIQMTQSPSSLSASVGDRVTITCKASQDVSTAVAWYQQKPGKAPKLLIYSASYRYTGVPSRFSG SGSGTDFTLTISSLQPEDFAVYYCQQHYITPLTFGQGTKLEIK (SEQ ID NO:1127); and b) VH CDR1, CDR2, and CDR3 present in a heavy chain variable region (VH) comprising the following amino acid sequence: QVQLVQSGAEVKKPGASVKVSCKASGYTFTTAGMQWVRQAPGQGLEWMGWINTHSGVPKYA EDFKGRVTISADTSTSTAYLQLSSLKSEDTAVYYCARSGFGSSYWYFDVWGQGTLVTVSS (SEQ ID NO:1128). In some cases, the V_{H} and V_{L} CDRs are as defined by Kabat (see, e.g., Table 1, above; and Kabat 1991). In some cases, the V_{H} and V_{L} CDRs are as defined by Chothia (see, e.g., Table 1, above; and Chothia 1987).

In some cases, an anti-TROP-2 antibody suitable for inclusion in a TMMP comprises: a) a VL region comprising an amino acid sequence having at least 90%, at least 95%, at least 98%, at least 99%, or 100%, amino acid sequence identity to the following amino acid sequence: DIQMTQSPSSLSASVGDRVTITCKASQDVSTAVAWYQQKPGKAPKLLIYSASYRYTGVPSRFSG SGSGTDFTLTISSLQPEDFAVYYCQQHYITPLTFGQGTKLEIK (SEQ ID NO:1127); and b) a VH region comprising an amino acid sequence having at least 90%, at least 95%, at least 98%, at least 99%, or 100%, amino acid sequence identity to the following amino acid sequence:

(SEQ ID NO: 1128)
QVQLVQSGAEVKKPGASVKVSCKASGYTFTTAGMQWVRQAPGQGLEWMGWI
NTHSGVPKYAEDFKGRVTISADTSTSTAYLQLSSLKSEDTAVYYCARSGFG
SSYWYFDVWGQGTLVTVSS.

In some cases, an anti-TROP-2 antibody suitable for inclusion in a TMMP is a scFv comprising, in order from N-terminus to C-terminus: a) a VL region comprising an amino acid sequence having at least 90%, at least 95%, at least 98%, at least 99%, or 100%, amino acid sequence identity to the following amino acid sequence: DIQMTQSPSSLSASVGDRVTITCKASQDVSTAVAWYQQKPGKAPKLLIYSASYRYTGVPSRFSG SGSGTDFTLTISSLQPEDFAVYYCQQHYITPLTFGQGTKLEIK (SEQ ID NO:1127); b) a peptide linker; and c) a VH region comprising an amino acid sequence having at least 90%, at least 95%, at least 98%, at least 99%, or 100%, amino acid sequence identity to the following amino acid sequence: QVQLVQSGAEVKKPGASVKVSCKASGYTFTTAGMQWVRQAPGQGLEWMGWINTHSGVPKYA EDFKGRVTISADTSTSTAYLQLSSLKSEDTAVYYCARSGFGSSYWYFDVWGQGTLVTVSS (SEQ ID NO:1128). In some cases, the peptide linker comprises the amino acid sequence (GGGGS)n (SEQ ID NO:934), where n is an integer from 1 to 10 (e.g., where n is 1, 2, 3, 4, 5, 6, 7, 8, 9, or 10). In some cases, the peptide linker comprises the amino acid sequence GGGGSGGGGSGGGGS (SEQ ID NO:875) and has a length of 15 amino acids.

In some cases, an anti-TROP-2 antibody suitable for inclusion in a TMMP is a scFv comprising, in order from N-terminus to C-terminus: a) a VH region comprising an amino acid sequence having at least 90%, at least 95%, at least 98%, at least 99%, or 100%, amino acid sequence identity to the following amino acid sequence: QVQLVQSGAEVKKPGASVKVSCKASGYTFTTAGMQWVRQAPGQGLEWMGWINTHSGVPKYA EDFKGRVTISADTSTSTAYLQLSSLKSEDTAVYYCARSGFGSSYWYFDVWGQGTLVTVSS (SEQ ID NO:1128); b) a peptide linker; and c) a VL region comprising an amino acid sequence having at least 90%, at least 95%, at least 98%, at least 99%, or 100%, amino acid sequence identity to the following amino acid sequence: DIQMTQSPSSLSASVGDRVTITCKASQDVSTAVAWYQQKPGKAPKLLIYSASYRYTGVPSRFSG SGSGTDFTLTISSLQPEDFAVYYCQQHYITPLTFGQGTKLEIK (SEQ ID NO:1127). In some cases, the peptide linker comprises the amino acid sequence (GGGGS)n (SEQ ID NO:934), where n is an integer from 1 to 10 (e.g., where n is 1, 2, 3, 4, 5, 6, 7, 8, 9, or 10). In some cases, the peptide linker comprises the amino acid sequence GGGGSGGGGSGGGGS (SEQ ID NO:875) and has a length of 15 amino acids.

### Anti-BCMA

Anti-BCMA (B-cell maturation antigen) antibodies are known in the art; and the VH and VL, or the VH and VL CDRs, of any anti-BCMA antibody can be used in a TMMP. See, e.g., WO 2014/089335; US 2019/0153061; and WO 2017/093942.

In some cases, an anti-BCMA antibody comprises: a) a light chain comprising an amino acid sequence having at least 90%, at least 95%, at least 98%, at least 99%, or 100%, amino acid sequence identity to the following amino acid sequence:
QSVLTQPPSASGTPGQRVTISCSGSSSNIGSNTVNWYQQLPGTAPKLLIFNYHQRP SGVPDRFSGSKSGSSASLAISGLQSEDEADYYCAAWDDSLNGWVFGGGTKLTVLGQPKAAPSV TLFPPSSEELQANKATLVCLISDFYPGAVTVAWKADSSPVKAGVETTTPDSKQSNNKYAASSYL SLTPEQWKSHRSYSCQVTHEGSTVEKTVAPTECS (SEQ ID NO:1129); and
b) a heavy chain comprising an amino acid sequence having at least 90%, at least 95%, at least 98%, at least 99%, or 100%, amino acid sequence identity to the following amino acid sequence:

(SEQ ID NO: 1130)
EVQLVESGGGLVKPGGSLRLSCAASGFTFGDYALSWFRQAPGKGLEWVGVS
RSKAYGGTTDYAASVKGRFTISRDDSKSTAYLQMNSLKTEDTAVYYCASSG
YSSGWTPFDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLFPPKPKD
TLMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSDG
SFFLYSKLTVDKSRWQQGNVFSCSVMHEALHNHYTQKSLSLSPGK.

In some cases, an anti-BCMA antibody comprises a VL present in the light chain amino acid sequence provided above; and a VH present in the heavy chain amino acid sequence provided above. For example, an anti-BCMA antibody can comprise: a) a VL comprising an amino acid sequence having at least 90%, at least 95%, at least 98%, at least 99%, or 100%, amino acid sequence identity to the amino acid sequence:
QSVLTQPPSASGTPGQRVTISCSGSSSNIGSNTVNWYQQLPGTAPKLLIFNYHQRP SGVPDRFSGSKSGSSASLAISGLQSEDEADYYCAAWDDSLNGWVFGGGTKLTVLG (SEQ ID NO:1131); and b) a VH comprising an amino acid sequence having at least 90%, at least 95%, at least 98%, at least 99%, or 100%, amino acid sequence identity to the amino acid sequence:
EVQLVESGGGLVKPGGSLRLSCAASGFTFGDYALSWFRQAPGKGLEWVGVSRS KAYGGTTDYAASVKGRFTISRDDSKSTAYLQMNSLKTEDTAVYYCASSGYSSGWTPFDYWGQ GTLVTVSSASTKGPSV (SEQ ID NO:1132).

In some cases, an anti-BCMA antibody comprises VL CDR1, VL CDR2, and VL CDR3 present in the light chain amino acid sequence provided above; and VH CDR1, CDR2, and CDR3 present in the heavy chain amino acid sequence provided above. In some cases, the V_{H} and V_{L} CDRs are as defined by Kabat (see, e.g., Table 1, above; and Kabat 1991). In some cases, the V_{H} and V_{L} CDRs are as defined by Chothia (see, e.g., Table 1, above; and Chothia 1987).

For example, an anti-BCMA antibody can comprise a VL CDR1 having the amino acid sequence SSNIGSNT (SEQ ID NO:1133), a VL CDR2 having the amino acid sequence NYH, a VL CDR3 having the amino acid sequence AAWDDSLNGWV (SEQ ID NO:1134)), a VH CDR1 having the amino acid sequence GFTFGDYA (SEQ ID NO: 1135), a VH CDR2 having the amino acid sequence SRSKAYGGTT (SEQ ID NO:1136), and a VH CDR3 having the amino acid sequence ASSGYSSGWTPFDY (SEQ ID NO:1137).

An anti-BCMA antibody can be a scFv. As one non-limiting example, an anti-BCMA scFv can comprise the following amino acid sequence:

(SEQ ID NO: 1138)
QVQLVQSGAEVKKPGSSVKVSCKASGGTFSNYWMHWVRQAPGQGLEWMGAT
YRGHSDTYYNQKFKGRVTITADKSTSTAYMELSSLRSEDTAVYYCARGAIY
NGYDVLDNWGQGTLVTVSSGGGGSGGGGSGGGGSGGGGSDIQMTQSPSSLS
ASVGDRVTITCSASQDISNYLNWYQQKPGKAPKLLIYYTSNLHSGVPSRFS
GSGSGTDFTLTISSLQPEDFATYYCQQYRKLPWTFGQGTKLEIKR.

As another example, an anti-BCMA scFv can comprise the following amino acid sequence:

(SEQ ID NO: 1139)
DIQMTQSPSSLSASVGDRVTITCSASQDISNYLNWYQQKPGKAPKLLIYYT
SNLHSGVPSRFSGSGSGTDFTLTISSLQPEDFATYYCQQYRKLPWTFGQGT
KLEIKRGGGGSGGGGSGGGGSGGGGSQVQLVQSGAEVKKPGSSVKVSCKAS
GGTFSNYWMHWVRQAPGQGLEWMGATYRGHSDTYYNQKFKGRVTITADKST
STAYMELSSLRSEDTAVYYCARGAIYNGYDVLDNWGQGTLVTVSS

In some cases, an anti-BCMA antibody can comprise a VL CDR1 having the amino acid sequence SASQDISNYLN (SEQ ID NO:1140); a VL CDR2 having the amino acid sequence YTSNLHS (SEQ ID NO:1141); a VL CDR3 having the amino acid sequence QQYRKLPWT (SEQ ID NO:1142); a VH CDR1 having the amino acid sequence NYWMH (SEQ ID NO:1143); a VH CDR2 having the amino acid sequence ATYRGHSDTYYNQKFKG (SEQ ID NO:1144); and a VH CDR3 having the amino acid sequence GAIYNGYDVLDN (SEQ ID NO:1145).

In some cases, an anti-BCMA antibody comprises: a) a light chain comprising an amino acid sequence having at least 90%, at least 95%, at least 98%, at least 99%, or 100%, amino acid sequence identity to the following amino acid sequence:

(SEQ ID NO: 1146)
DIQMTQSPSSLSASVGDRVTITCSASQDISNYLNWYQQKPGKAPKLLIYYT
SNLHSGVPSRFSGSGSGTDFTLTISSLQPEDFATYYCQQYRKLPWTFGQGT
KLEIKR.

In some cases, an anti-BCMA antibody comprises: a) a heavy chain comprising an amino acid sequence having at least 90%, at least 95%, at least 98%, at least 99%, or 100%, amino acid sequence identity to the following amino acid sequence:

(SEQ ID NO: 1147)
QVQLVQSGAEVKKPGSSVKVSCKASGGTFSNYWMHWVRQAPGQGLEWMGAT
YRGHSDTYYNQKFKGRVTITADKSTSTAYMELSSLRSEDTAVYYCARGAIY
DGYDVLDNWGQGTLVTVSS.

In some cases, an anti-BCMA antibody (e.g., an antibody referred to in the literature as belantamab) comprises a light chain comprising the amino acid sequence: DIQMTQSPSSLSASVGDRVTITCSASQDISNYLNWYQQKPGKAPKLLIYYTSNLHSGVPSRFSGS GSGTDFTLTISSLQPEDFATYYCQQYRKLPWTFGQGTKLEIKR (SEQ ID NO:1146); and a heavy chain comprising the amino acid sequence:

(SEQ ID NO: 1147)
QVQLVQSGAEVKKPGSSVKVSCKASGGTFSNYWMHWVRQAPGQGLEWMGAT
YRGHSDTYYNQKFKGRVTITADKSTSTAYMELSSLRSEDTAVYYCARGAIY
DGYDVLDNWGQGTLVTVSS.

In some cases, the anti-BCMA antibody has a cancer chemotherapeutic agent linked to the antibody. For example, in some cases, the anti-BCMA antibody is GSK2857916 (belantamab-mafodotin), where monomethyl auristatin F (MMAF) is linked via a maleimidocaproyl linker to the anti-BCMA antibody belantamab.

### Anti-MUC1

In some cases, a TTP present in a TMMP is an antibody specific for MUC1. For example, a TTP can be specific for a MUC1 polypeptide present on a cancer cell. In some cases, the TTP is specific for the cleaved form of MUC1; see, e.g., Fessler et al. (2009) Breast Cancer Res. Treat. 118:113. In some cases, the TTP is an antibody specific for a glycosylated MUC1 peptide; see, e.g., Naito et al. (2017) ACS Omega 2:7493; and U.S. Pat. No. 10,017,580.

As one non-limiting example, a TTP can be a single-chain Fv specific for MUC1. See, e.g., Singh et al. (2007) Mol. Cancer Ther. 6:562; Thie et al. (2011) PloSOne 6:e15921; Imai et al. (2004) Leukemia 18:676; Posey et al. (2016) Immunity 44:1444; EP3130607; EP3164418; WO 2002/044217; and US 2018/0112007. In some cases, a TTP is a scFv specific for the MUCI peptide VTSAPDTRPAPGSTAPPAHG (SEQ ID NO:1148). In some cases, a TTP is a scFv specific for the MUCI peptide SNIKFRPGSVVVQLTLAFREGTINVHDVETQFNQYKTEAASRY (SEQ ID NO:1149). In some cases, a TTP is a scFv specific for the MUC1 peptide SVVVQLTLAFREGTINVHDVETQFNQYKTEAASRY (SEQ ID NO:1150). In some cases, a TTP is a scFv specific for the MUCI peptide LAFREGTINVHDVETQFNQY (SEQ ID NO:1151). In some cases, a TTP is a scFv specific for the MUCI peptide SNIKFRPGSVVVQLTLAAFREGTIN (SEQ ID NO:1152).

As an example, an anti-MUCI antibody can comprise: a VH CDR1 having the amino acid sequence RYGMS (SEQ ID NO:1153); a VH CDR2 having the amino acid sequence TISGGGTYIYYPDSVKG (SEQ ID NO:1154); a VH CDR3 having the amino acid sequence DNYGRNYDYGMDY (SEQ ID NO:1155); a VL CDR1 having the amino acid sequence SATSSVSYIH (SEQ ID NO:1156); a VL CDR2 having the amino acid sequence STSNLAS (SEQ ID NO:1157); and a VL CDR3 having the amino acid sequence QQRSSSPFT (SEQ ID NO:1158). See, e.g., US 2018/0112007.

As another example, an anti-MUCI antibody can comprise a VH CDR1 having the amino acid sequence GYAMS (SEQ ID NO:1159); a VH CDR2 having the amino acid sequence TISSGGTYIYYPDSVKG (SEQ ID NO:1160); a VH CDR3 having the amino acid sequence LGGDNYYEYFDV (SEQ ID NO:1161); a VL CDR1 having the amino acid sequence RASKSVSTSGYSYMH (SEQ ID NO:1162); a VL CDR2 having the amino acid sequence LASNLES (SEQ ID NO:1163); and a VL CDR3 having the amino acid sequence QHSRELPFT (SEQ ID NO:1164). See, e.g., US 2018/0112007.

As another example, an anti-MUCI antibody can comprise a VH CDR1 having the amino acid sequence DYAMN (SEQ ID NO:1165); a VH CDR2 having the amino acid sequence VISTFSGNINFNQKFKG (SEQ ID NO:1166); a VH CDR3 having the amino acid sequence SDYYGPYFDY (SEQ ID NO:1167); a VL CDR1 having the amino acid sequence RSSQTIVHSNGNTYLE (SEQ ID NO:1168); a VL CDR2 having the amino acid sequence KVSNRFS (SEQ ID NO:1169); and a VL CDR3 having the amino acid sequence (FQGSHVPFT (SEQ ID NO:1170). See, e.g., US 2018/0112007.

As another example, an anti-MUC1 antibody can comprise a VH CDR1 having the amino acid sequence GYAMS (SEQ ID NO:1159); a VH CDR2 having the amino acid sequence TISSGGTYIYYPDSVKG (SEQ ID NO:1160); a VH CDR3 having the amino acid sequence LGGDNYYEY (SEQ ID NO:1171); a VL CDR1 having the amino acid sequence TASKSVSTSGYSYMH (SEQ ID NO:1172); a VL CDR2 having the amino acid sequence LVSNLES (SEQ ID NO:1173); and a VL CDR3 having the amino acid sequence QHIRELTRSE (SEQ ID NO:1174). See, e.g., US 2018/0112007.

### Anti-MUC16

In some cases, a TTP present in a TMMP is an antibody specific for MUC16 (also known as CA125). See, e.g., Yin et al. (2002) Int. J. Cancer 98:737. For example, a TTP can be specific for a MUC16 polypeptide present on a cancer cell. See, e.g., US 2018/0118848; and US 2018/0112008. In some cases, a MUC16-specific TTP is a scFv. In some cases, a MUC16-specific TTP is a nanobody.

As one example, an anti-MUC16 antibody can comprise a VH CDR1 having the amino acid sequence GFTFSNYY (SEQ ID NO:1175); a VH CDR2 having the amino acid sequence ISGRGSTI (SEQ ID NO:1176); a VH CDR3 having the amino acid sequence VKDRGGYSPY (SEQ ID NO:1177); a VL CDR1 having the amino acid sequence QSISTY (SEQ ID NO:1178); a VL CDR2 having the amino acid sequence TAS; and a VL CDR3 having the amino acid sequence QQSYSTPPIT (SEQ ID NO:1179). See, e.g., US 2018/0118848.

### Anti-Claudin-18.2

In some cases, a TTP present in a TMMP is an antibody specific for claudin-18 isoform 2 (“claudin-18.2”). See, e.g., WO 2013/167259. In some cases, a claudin-18.2-specific TTP is a scFv. In some cases, a claudin-18.2-specific TTP is a nanobody. In some cases, a TPP present in a TMMP is an antibody specific for TEDEVQSYPSKHDYV (SEQ ID NO:1058) or EVQSYPSKHDYV (SEQ ID NO:1059).

As one example, an anti-claudin-18.2 antibody can comprise a VH CDR1 having the amino acid sequence GYTFTDYS (SEQ ID NO:1180); a VH CDR2 having the amino acid sequence INTETGVP (SEQ ID NO:1181); a VH CDR3 having the amino acid sequence ARRTGFDY (SEQ ID NO:1182); a VL CDR1 having the amino acid sequence KNLLHSDGITY (SEQ ID NO:1183); a VL CDR2 having the amino acid sequence RVS; and a VL CDR3 having the amino acid sequence VQVLELPFT (SEQ ID NO:1184).

As another example, an anti-claudin-18.2 antibody can comprise a VH CDR1 having the amino acid sequence GFTFSSYA (SEQ ID NO:1185); a VH CDR2 having the amino acid sequence ISDGGSYS (SEQ ID NO:1186); a VH CDR3 having the amino acid sequence ARDSYYDNSYVRDY (SEQ ID NO:1187); a VL CDR1 having the amino acid sequence QDINTF (SEQ ID NO:1188); a VL CDR2 having the amino acid sequence RTN; and a VL CDR3 having the amino acid sequence LQYDEFPLT (SEQ ID NO:1189).

### mAb FUSIONS

In embodiments, a TMMP can comprise a heterodimer, wherein: a) the first polypeptide of the heterodimer comprises i) a single-chain polypeptide comprising a peptide epitope (e.g., a viral epitope); a β2M polypeptide; and an MHC class I heavy chain polypeptide, and ii) an antibody heavy chain polypeptide; and wherein b) the second polypeptide of the heterodimer comprises an antibody light chain polypeptide, and wherein either the first polypeptide or the second polypeptide comprises one or more immunomodulatory polypeptides, e.g., on the N-terminus of the antibody light chain polypeptide, on the C-terminus of the antibody light chain polypeptide, or on the C-terminus of the antibody heavy chain polypeptide. A TMMP can thus comprise a heterodimer comprising: a) a first polypeptide comprising: i) a peptide epitope (e.g., a viral epitope); ii) a β2M polypeptide; iii) an MHC class I heavy chain polypeptide; and iv) an antibody heavy chain polypeptide (e.g., an antibody heavy chain comprising, in order from N-terminus to C-terminus: i) a VH polypeptide; ii) an Ig CH1 polypeptide; and iii) an IgG Fc polypeptide); and b) a second polypeptide comprising: i) one or more immunomodulatory polypeptides; and ii) an antibody light chain polypeptide (e.g., an antibody light chain comprising, in order from N-terminus to C-terminus: i) a VL polypeptide; and an Ig kappa light chain constant region polypeptide). A TMMP can comprise a heterodimer comprising: a) a first polypeptide comprising: i) a peptide epitope (e.g., a viral epitope); ii) a β2M polypeptide; iii) an MHC class I heavy chain polypeptide; iv) an antibody heavy chain polypeptide (e.g., an antibody heavy chain comprising, in order from N-terminus to C-terminus: i) a VH polypeptide; ii) an Ig CH1 polypeptide; and iii) an IgG Fc polypeptide); and v) one or more immunomodulatory polypeptides; and b) a second polypeptide comprising an antibody light chain polypeptide (e.g., an antibody light chain comprising, in order from N-terminus to C-terminus: i) a VL polypeptide; and an Ig kappa light chain constant region polypeptide). A TMMP can comprise a heterodimer comprising: a) a first polypeptide comprising: i) a peptide epitope (e.g., a viral epitope); ii) a β2M polypeptide; iii) an MHC class I heavy chain polypeptide; and iv) an antibody heavy chain polypeptide (e.g., an antibody heavy chain comprising, in order from N-terminus to C-terminus: i) a VH polypeptide; ii) an Ig CH1 polypeptide; and iii) an IgG Fc polypeptide); and b) a second polypeptide comprising: i) an antibody light chain polypeptide (e.g., an antibody light chain comprising, in order from N-terminus to C-terminus: i) a VL polypeptide; and an Ig kappa light chain constant region polypeptide); and ii) one or more immunomodulatory polypeptides. Such heterodimers can dimerize (e.g., form homodimers of two of the heterodimers by the formation of disulfide bonds that form between the antibody heavy chain polypeptides of each heterodimer). Such TMMPs are referred to herein as “monoclonal antibody fusions” or “mAb fusions.” The VH polypeptide and the VL polypeptide are on separate polypeptides of the heterodimer; together, the VH polypeptide and the VL polypeptide bind to a cancer epitope and therefore constitute a TTP.

The following are non-limiting examples of mAb fusions.

As one example, a TMMP can comprise: a) a first polypeptide comprising the amino acid sequence depicted in FIG. 24A; and b) a second polypeptide comprising the amino acid sequence depicted in FIG. 24B. As an example, the SARS-CoV-2 peptide can have the amino acid sequence YLQPRTFLL (SEQ ID NO:218).

As another example, a TMMP can comprise: a) a first polypeptide comprising the amino acid sequence depicted in FIG. 25A; and b) a second polypeptide comprising the amino acid sequence depicted in FIG. 25B. As an example, the SARS-CoV-2 peptide can have the amino acid sequence YLQPRTFLL (SEQ ID NO:218).

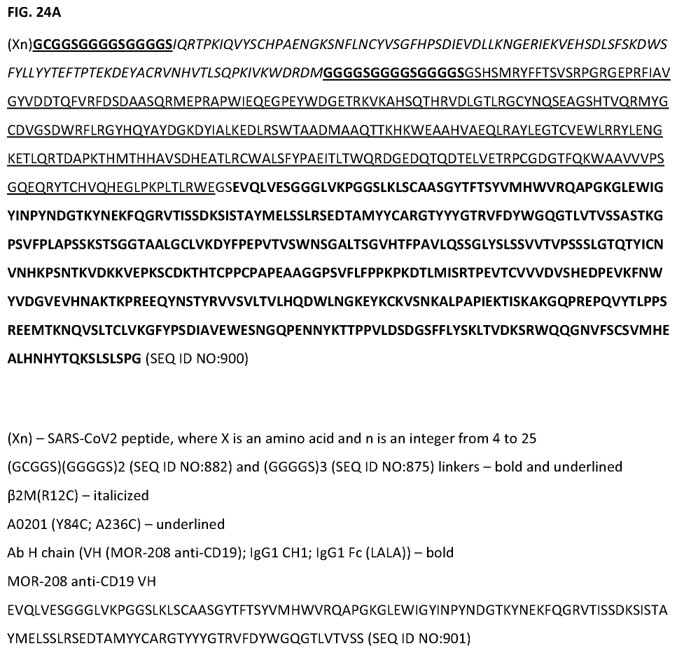

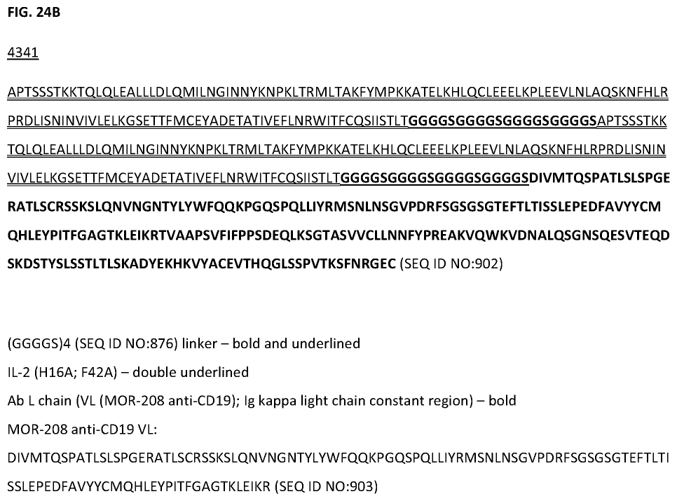

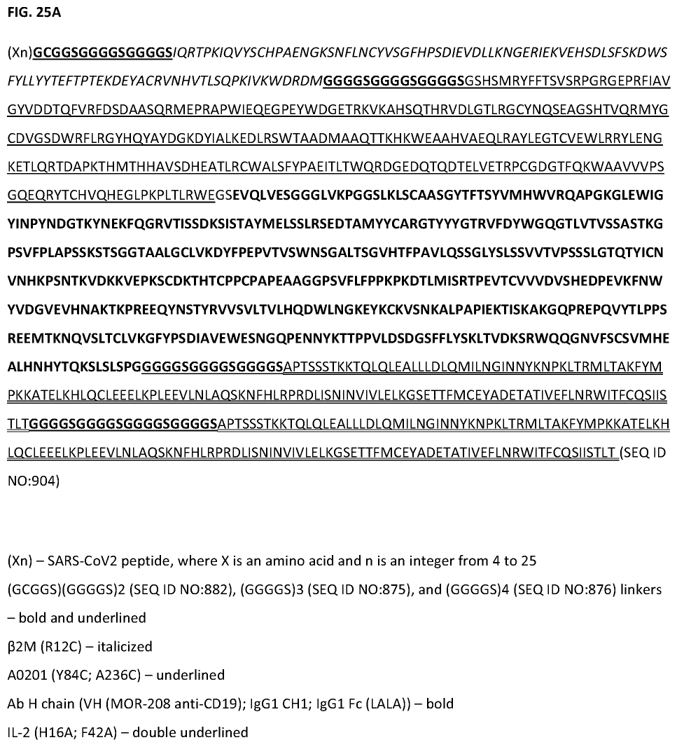

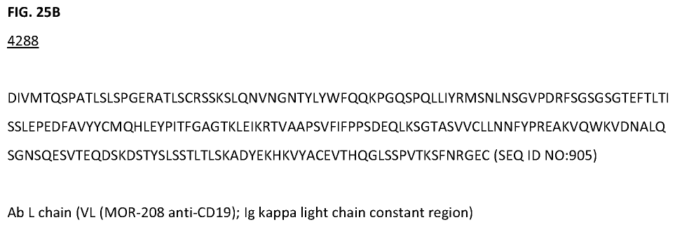

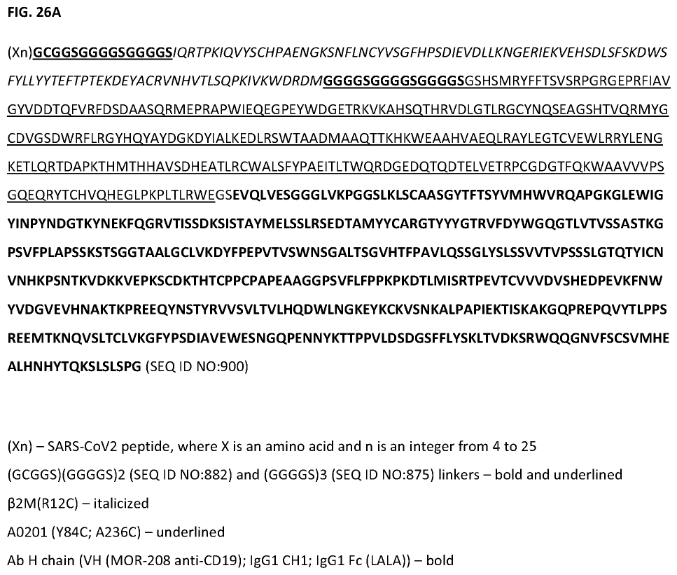

As another example, a TMMP can comprise: a) a first polypeptide comprising the amino acid sequence depicted in FIG. 26A; and b) a second polypeptide comprising the amino acid sequence depicted in FIG. 26B. As an example, the SARS-CoV-2 peptide can have the amino acid sequence YLQPRTFLL (SEQ ID NO:218).

### Single-Chain T-Cell Receptors

As noted above, in some cases, a TTP present in a TMMP is a scTCR. A TTP can be a scTCR specific for a peptide/HLA complex on the surface of a cancer cell, where the peptide can be a cancer-associated peptide (e.g., a peptide of a cancer-associated antigen). Amino acid sequences of scTCRs specific for cancer-associated peptides bound to an HLA complex are known in the art. See, e.g., US 2019/0135914; US 2019/0062398; and US 2018/0371049.

A scTCR includes an alpha chain variable region (Vα) and a beta chain variable region (Vβ) covalently linked through a suitable peptide linker sequence. For example, the Vα can be covalently linked to the VP through a suitable peptide linker (L) sequence fused to the C-terminus of the Vα and the N-terminus of the Vβ. An scTCR can have the structure Vα-L-Vβ. An scTCR can have the structure VR-L-Va. An scTCR can also comprise a constant domain (also referred to as constant region). In some cases, an scTCR comprises, in order from N-terminus to C-terminus: i) a TCR α chain variable domain polypeptide; ii) a peptide linker; iii) a TCR β chain variable domain polypeptide; and iv) a TCR β chain constant region extracellular domain polypeptide. In some cases, an scTCR comprises, in order from N-terminus to C-terminus: i) a TCR β chain variable domain polypeptide; ii) a peptide linker; iii) a TCR α chain variable domain polypeptide; and iv) a TCR α chain constant region extracellular domain polypeptide.

Amino acid sequences of scTCRs specific for peptide/HLA complexes, where the peptide is a cancer-associated peptide, are known in the art. See, e.g., US 2019/0135914; US 2019/0062398; US 2018/0371049; US 2019/0144563; and US 2019/0119350.

For example, a scTCR can be specific for an NY-ESO epitope such as an SLLMWITQC (SEQ ID NO:1190) peptide bound to an HLA complex comprising an HLA-A*0201 heavy chain and a β2M polypeptide. As an example, such an scTCR can comprise: i) a TCR α chain variable region comprising an amino acid sequence having at least 90%, at least 95%, at least 98%, at least 99%, or 100%, amino acid sequence identity to the amino acid sequence: MQEVTQIPAALSVPEGENLVLNCSFTDSAIYNLQWFRQDPGKGLTSLLLIQSSQREQTSGRLNAS LDKSSGRSTLYIAASQPGDSATYLCAVRPTSGGSYIPTFGRGTSLIVHPY (SEQ ID NO:1191), where amino acid 20 can be V or A; amino acid 51 can be Q, P, S, T, or M; amino acid 52 can be S, P, F, or G, amino acid 53 can be S, W, H, or T; amino acid 94 can be P, H, or A; amino acid 95 can be T, L, M, A, Q, Y, E, I, F, V, N, G, S, D, or R; amino acid 96 can be S, L, T, Y, I, Q, V, E, A, W, R, G, H, D, or K; amino acid 97 can be G, D, N, V, S, T, or A; amino acid 98 can be G, P, H, S, T, W, or A; amino acid 99 can be S, T, Y, D, H, V, N, E, G, Q, K, A, I, or R; amino acid 100 can be Y, F, M, or D; amino acid 101 can be I, P, T, or M; and amino acid 103 can be T or A; and ii) a TCR β chain variable region comprising an amino acid sequence having at least 90%, at least 95%, at least 98%, at least 99%, or 100%, amino acid sequence identity to the amino acid sequence: MGVTQTPKFQVLKTGQSMTLQCAQDMNHEYMSWYRQDPGMGLRLIHYSVGAGITDQGEVPN GYNVSRSTTEDFPLRLLSAAPSQTSVYFCASSYVGNTGELFFGEGSRLTVL (SEQ ID NO:1192), where amino acid 18 can be M or V; amino acid 50 can be G, V, or I; amino acid 52 can be G or Q; amino acid 53 can be I, T, or M; amino acid 55 can be D or R; amino acid 56 can be Q or R; amino acid 70 can be T or I; amino acid 94 can be Y, N, or F; amino acid 95 can be V or L; and amino acid 97 can be N, G, or D. For example, in some cases, a scTCR can comprise: i) a TCR α chain variable region comprising the amino acid sequence: MQEVTQIPAALSVPEGENLVLNCSFTDSAIYNLQWFRQDPGKGLTSLLLIMSHQREQTSGRLNA SLDKSSGRSTLYIAASQPGDSATYLCAVRPTSGGSYIPTFGRGTSLIVHPY (SEQ ID NO:1193); and a TCR β chain variable region comprising the amino acid sequence:

(SEQ ID NO: 1194)
MGVTQTPKFQVLKTGQSMTLQCAQDMNHEYMSWYRQDPGMGLRLIHYSVSA
GITDQGEVPNGYNVSRSTTEDFPLRLLSAAPSQTSVYFCASSYVGNTGELF
FGEGSRLTVL.

As another example, a scTCR can be specific for an HPV epitope (e.g., an HPV peptide of the amino acid sequence YIIFVYIPL (HPV 16 E5₆₃₋₇₁; SEQ ID NO:1195), KLPQLCTEL (HPV 16 E6₁₁₋₁₉; SEQ ID NO:1042), TIHEIILECV (HPV 16 E6; SEQ ID NO:1196), YMLDLQPET (HPV 16 E7₁₁₋₁₉; SEQ ID NO:1049), TLGIVCPI (HPV 16 E7₈₆₋₉₃) (SEQ ID NO:1055), KCIDFYSRI (HPV 18 E6₆₇₋₇₅; SEQ ID NO:1197), or FQQLFLNTL (HPV 18 E7₈₆₋₉₄; SEQ ID NO:1198)) bound to an HLA complex comprising an HL heavy chain and a β2M polypeptide. As an example, such an scTCR can comprise: i) a TCR α chain variable region comprising an amino acid sequence having at least 90%, at least 95%, at least 98%, at least 99%, or 100%, amino acid sequence identity to the amino acid sequence: METLLGLLILQLQLQWVSSKQEVTQIPAALSVPEGENLVLNCSFTDSAIYNLQWFRQDPGKGLT SLLLIQSSQREQTSGRLNASLDKSSGRSTLYIAASQPGDSATYLCAVRETSGSRLTFGEGTQLTVN PD(SEQ ID NO:1199); and ii) a TCR f chain variable region comprising an amino acid sequence having at least 90%, at least 95%, at least 98%, at least 99%, or 100%, amino acid sequence identity to the amino acid sequence:

(SEQ ID NO: 1200)
MGIRLLCRVAFCFLAVGLVDVKVTQSSRYLVKRTGEKVFLECVQDMDHENM
FWYRQDPGLGLRLIYFSYDVKMKEKGDIPEGYSVSREKKERFSLILESAST
NQTSMYLCASSFWGRSTDTQYFGPGTRLTVL.

### Contrast Agents

In some cases, the TTP of a TMMP comprises a contrast agent or a radiolabel, where the contrast agent facilitates imaging of a tumor to which the TMMP binds.

Suitable agents include computed tomography (CT), a positron emission tomography (PET), and single photon emission computed tomography (SPECT) radiotracers. Suitable PET/SPECT contrast agents include, e.g., a positron emitter, for example ¹¹C, ¹³N, ¹⁸F, ⁸²Ru, and ¹⁵O. Iodinated CT contrast agents can be used. Suitable contrast agents include gadolinium (Gd), dysprosium, and iron. Gd chelates, such as Gd diethylene triamine pentaacetic acid (GdDTPA), Gd tetraazacyclododecanetetraacetic acid (GdDOTA), polylysine-Gd chelates, and derivatives thereof, can be used. Suitable radioisotopes include ¹²³I (iodine), ¹⁸F (fluorine), ⁹⁹Tc (technetium), ¹¹¹In (indium), and ⁶⁷Ga (gallium).

### HLA/Peptide Binding Assays

Whether a given peptide (e.g., a peptide that comprises an epitope) binds a class I HLA (comprising an HLA heavy chain and a β2M polypeptide), and, when bound to the HLA complex, can effectively present an epitope to a TCR, can be determined using any of a number of well-known methods. Assays include binding assays and T-cell activation assays. See, e.g., published PCT Application WO 2020/243315 (Cue Biopharma, Inc.).

### Exemplary TMMPs

A TMMP comprises at least one heterodimer comprising: a) a first polypeptide comprising: i) a SARS-CoV-2 peptide; and ii) first MHC polypeptide; b) a second polypeptide comprising a second MHC polypeptide, c) at least one immunomodulatory polypeptide, where the first and/or the second polypeptide comprises the immunomodulatory polypeptide; d) an Ig Fc polypeptide or a non-Ig scaffold, where the first and/or the second polypeptide comprises the Ig Fc polypeptide or the non-Ig scaffold; and e) a tumor-targeting polypeptide (TTP), where the first and/or the second polypeptide comprises the TTP. These components can be arranged in any of a variety of configurations. See, e.g., FIG. 18A-18C and FIG. 28A-28C for non-limiting examples of such configurations.

Thus, in some cases, a TMMP comprises at least one heterodimer comprising: a) a first polypeptide comprising: i) a SARS-CoV-2 peptide; ii) first MHC class I polypeptide; and iii) at least one immunomodulatory polypeptide; b) a second polypeptide comprising a second MHC class I polypeptide; c) an Ig Fc polypeptide or a non-Ig scaffold, where the first and/or the second polypeptide comprises the Ig Fc polypeptide or the non-Ig scaffold; and d) a TTP, where the first and/or the second polypeptide comprises the TTP. In other instances, a TMMP comprises at least one heterodimer comprising: a) a first polypeptide comprising: i) a SARS-CoV-2 peptide; and ii) first MHC class I polypeptide; b) a second polypeptide comprising: i) a second MHC class I polypeptide; and ii) at least one immunomodulatory polypeptide; c) an Ig Fc polypeptide or a non-Ig scaffold, where the first and/or the second polypeptide comprises the Ig Fc polypeptide or the non-Ig scaffold; and d) a TTP, where the first and/or the second polypeptide comprises the TTP. In some cases, a TMMP comprises at least one heterodimer comprising: a) a first polypeptide comprising: i) a SARS-CoV-2 peptide; ii) first MHC class I polypeptide; and iii) at least one immunomodulatory polypeptide; b) a second polypeptide comprising: i) a second MHC class I polypeptide; and ii) at least one immunomodulatory polypeptide; c) an Ig Fc polypeptide or a non-Ig scaffold, where the first and/or the second polypeptide comprises the Ig Fc polypeptide or the non-Ig scaffold; and d) a TTP, where the first and/or the second polypeptide comprises the TTP. In some cases, the at least one immunomodulatory polypeptide is a wild-type immunomodulatory polypeptide. In other cases, the at least one immunomodulatory polypeptide is a variant immunomodulatory polypeptide that exhibits reduced affinity for a co-immunomodulatory polypeptide, compared to the affinity of a corresponding wild-type immunomodulatory polypeptide for the co-immunomodulatory polypeptide. In some cases, a TMMP comprises two immunomodulatory polypeptides, where the two immunomodulatory polypeptides have the same amino acid sequence. In some cases, the TTP is an antibody specific for a cancer-associated antigen, e.g., a cancer-associated antigen present on the surface of a cancer cell. In some cases, the TTP is a scFv or a nanobody. In some cases, the TTP is an antibody specific for a cancer-associated peptide/HLA complex (i.e., an HLA heavy chain and a β2M polypeptide) present on the surface of a cancer cell. In some cases, the TTP is a single-chain T-cell receptor (scTCR) specific for a cancer-associated antigen, e.g., a cancer-associated antigen present on the surface of a cancer cell.

For example, in some cases, a TMMP comprises: a) a first polypeptide comprising, in order from N-terminus to C-terminus: i) a SARS-CoV-2 peptide; ii) an optional linker; and iii) a β2M polypeptide; and b) a second polypeptide comprising, in order from N-terminus to C-terminus: i) a TTP; ii) an optional linker; iii) an MHC class I heavy chain polypeptide; iv) an optional linker; v) an Ig Fc polypeptide; vi) an optional linker; and vii) a MOD. In some cases, a TMMP comprises: a) a first polypeptide comprising, in order from N-terminus to C-terminus: i) a SARS-CoV-2 peptide; ii) a linker; and iii) a β2M polypeptide; and b) a second polypeptide comprising, in order from N-terminus to C-terminus: i) a TTP; ii) a linker; iii) an MHC class I heavy chain polypeptide; iv) a linker; v) an Ig Fc polypeptide; vi) a linker; and vii) a MOD. In some cases, a TMMP comprises: a) a first polypeptide comprising, in order from N-terminus to C-terminus: i) a SARS-CoV-2 peptide; ii) a linker; and iii) a β2M polypeptide; and b) a second polypeptide comprising, in order from N-terminus to C-terminus: i) a TTP; ii) a linker; iii) an MHC class I heavy chain polypeptide; iv) a linker; v) an Ig Fc polypeptide; vi) a linker; vii) a first MOD; viii) a linker; and ix) a second MOD. In some cases, the HLA heavy chain comprises an amino acid sequence having at least 95% amino acid sequence identity to an HLA-A*0201, an HLA-A*1101, an HLA-A*2402, or an HLA-A*3303 amino acid sequence depicted in any one of FIG. 3A-3E, FIG. 4A-4E, FIG. 5A-5E, and FIG. 6A-6E. In some cases, the HLA heavy chain comprises an amino acid sequence having at least 95% amino acid sequence identity to an HLA-E amino acid sequence depicted in any one of FIG. 16A-16D. In some cases, the HLA heavy chain comprises an amino acid sequence having at least 95% amino acid sequence identity to an HLA-G amino acid sequence depicted in any one of FIG. 17A-17D. In some cases, the HLA heavy chain polypeptide is an HLA-A*0201 polypeptide. In some cases, the HLA heavy chain polypeptide is an HLA-A*2402 polypeptide. In some cases, the HLA heavy chain polypeptide is an HLA-A*1101 polypeptide. In some cases, the HLA heavy chain polypeptide is an HLA-A*3303 polypeptide. In some cases, the HLA heavy chain polypeptide is an HLA-E polypeptide. In some cases, the HLA heavy chain polypeptide is an HLA-G polypeptide. In some cases, the Ig Fc polypeptide is a human IgG1 Fc polypeptide. In some cases, the Ig Fc polypeptide is an IgG1 Fc polypeptide comprising L234A and L235A substitutions. In some cases, the first and the second polypeptides are disulfide linked to one another. In some cases, the first and the second polypeptides are linked to one another by 2 disulfide bonds. In some cases, the immunomodulatory polypeptide comprises a wild-type amino acid sequence; in other cases, the immunomodulatory polypeptide is a variant, e.g., as described above. In some cases, the immunomodulatory polypeptide is a 4-1BBL polypeptide, e.g., a variant 4-1BBL polypeptide. In some cases, the immunomodulatory polypeptide is a variant IL-2 polypeptide. In some cases, the immunomodulatory polypeptide is a variant IL-2 polypeptide comprising H16A and F42A substitutions. In some cases, the immunomodulatory polypeptide is a variant IL-2 polypeptide comprising H16T and F42A substitutions. In some cases, the second polypeptide comprises a linker between the TTP and the MHC class I heavy chain, where the linker has a length of less than about 25 amino acids, less than about 20 amino acids, less than about 15 amino acids, less than about 10 amino acids, less than about 8 amino acids, or less than about 6 amino acids. In some cases, the second polypeptide comprises a rigid peptide linker between the Ig Fc polypeptide and the MOD. In some cases, the TTP is an antibody specific for a cancer-associated antigen, e.g., a cancer-associated antigen present on the surface of a cancer cell. In some cases, the TTP is a scFv or a nanobody. In some cases, the TTP is an antibody specific for a cancer-associated peptide/HLA complex (i.e., an HLA heavy chain and a β2M polypeptide) present on the surface of a cancer cell. In some cases, the TTP is a scTCR specific for a cancer-associated antigen, e.g., a cancer-associated antigen present on the surface of a cancer cell. In some cases, the SARS-CoV-2 peptide comprises the amino acid sequence YLQPRTFLL (SEQ ID NO:218) and has a length of 9 amino acids. In some cases, the SARS-CoV-2 peptide has an amino acid sequence depicted in FIG. 19. In some cases, the SARS-CoV-2 peptide comprises the amino acid sequence GTHWFVTQR (SEQ ID NO:332) and has a length of 9 amino acids. In some cases, the SARS-CoV-2 peptide comprises the amino acid sequence QYIKWPWYI (SEQ ID NO:400) and has a length of 9 amino acids.

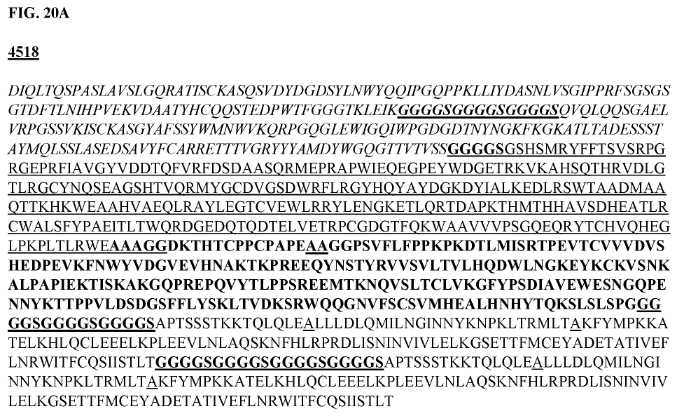

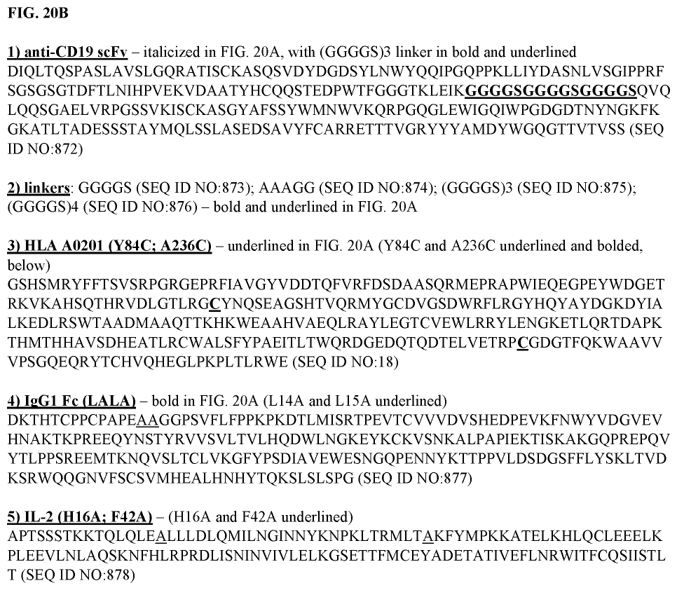

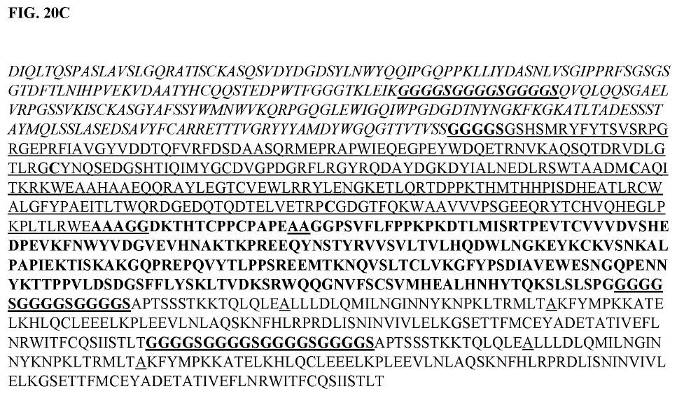

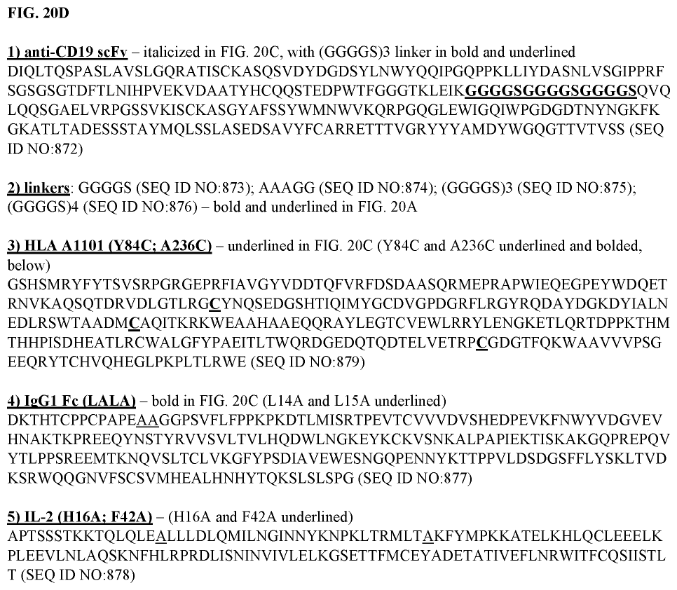

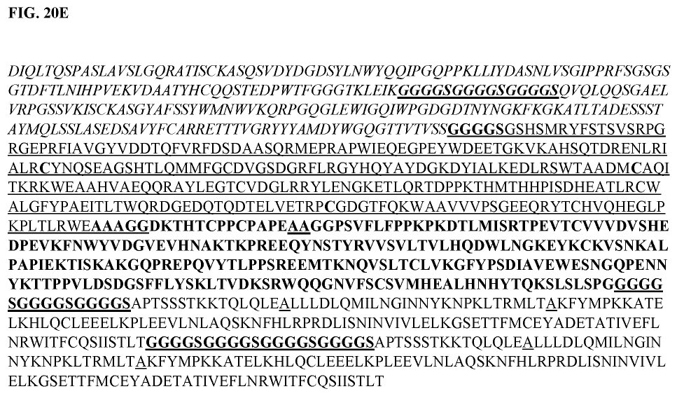

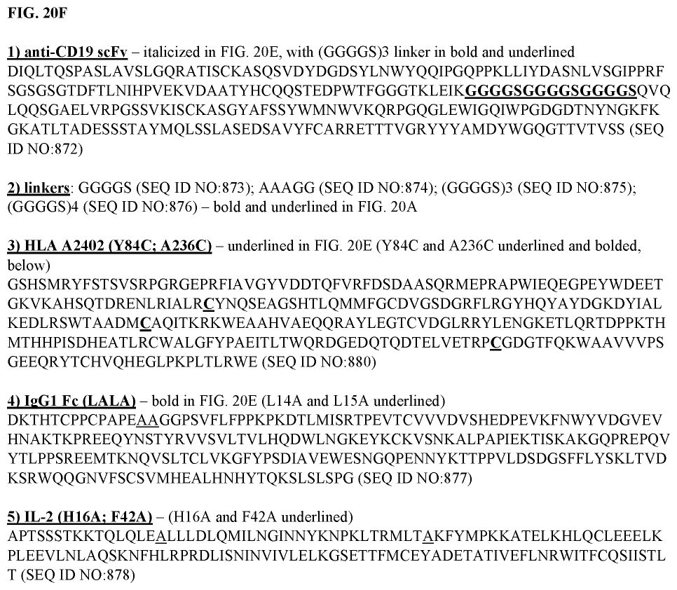

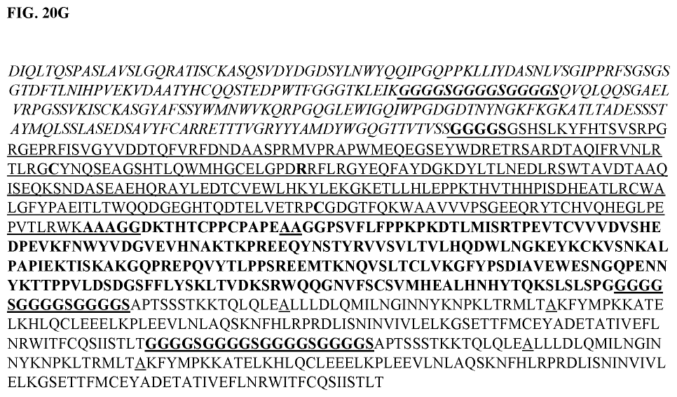

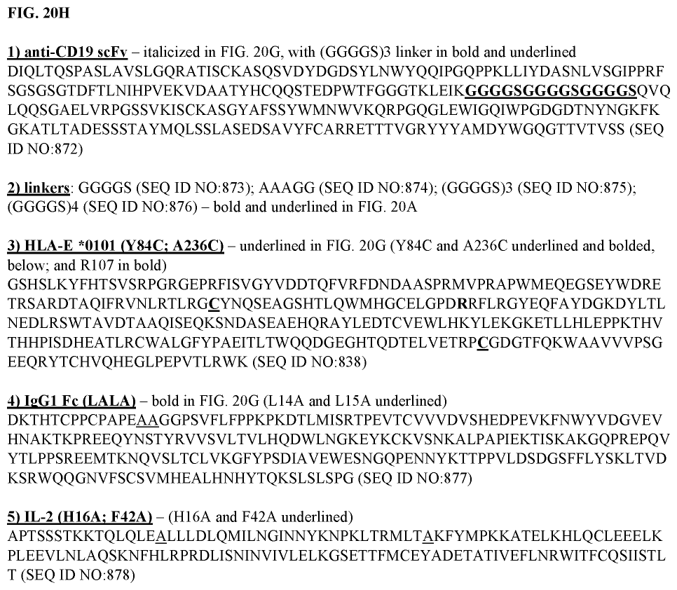

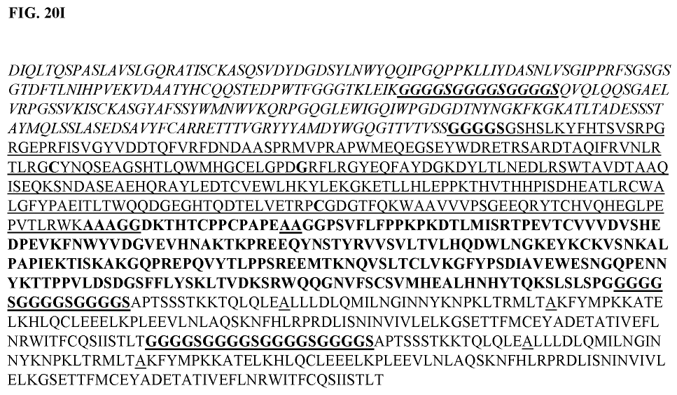

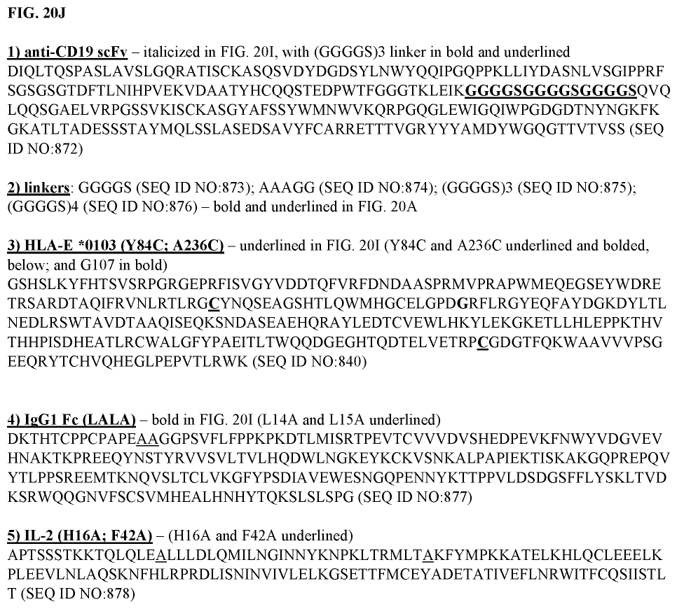

In some cases, a TMMP comprises an HLA-A*0201 polypeptide having an amino acid sequence having at least at least 70%, at least 75%, at least 80%, at least 85%, at least 90%, at least 95%, at least 98%, at least 99%, or 100%, amino acid sequence identity to the amino acid sequence of the HLA-A*0201 polypeptide depicted in FIG. 20B. In some cases, a TMMP comprises an IgG1 Fc polypeptide having an amino acid sequence having at least at least 70%, at least 75%, at least 80%, at least 85%, at least 90%, at least 95%, at least 98%, at least 99%, or 100%, amino acid sequence identity to the amino acid sequence of the IgG1 Fc polypeptide depicted in FIG. 20B. In some cases, a TMMP comprises an IL-2 polypeptide having an amino acid sequence having at least at least 70%, at least 75%, at least 80%, at least 85%, at least 90%, at least 95%, at least 98%, at least 99%, or 100%, amino acid sequence identity to the amino acid sequence of the IL-2 polypeptide depicted in FIG. 20B.

In some cases, a TMMP comprises an HLA-A*1101 polypeptide having an amino acid sequence having at least at least 70%, at least 75%, at least 80%, at least 85%, at least 90%, at least 95%, at least 98%, at least 99%, or 100%, amino acid sequence identity to the amino acid sequence of the HLA-A*1101 polypeptide depicted in FIG. 20D. In some cases, a TMMP comprises an IgG1 Fc polypeptide having an amino acid sequence having at least at least 70%, at least 75%, at least 80%, at least 85%, at least 90%, at least 95%, at least 98%, at least 99%, or 100%, amino acid sequence identity to the amino acid sequence of the IgG1 Fc polypeptide depicted in FIG. 20D. In some cases, a TMMP comprises an IL-2 polypeptide having an amino acid sequence having at least at least 70%, at least 75%, at least 80%, at least 85%, at least 90%, at least 95%, at least 98%, at least 99%, or 100%, amino acid sequence identity to the amino acid sequence of the IL-2 polypeptide depicted in FIG. 20D.

In some cases, a TMMP comprises an HLA-A*2402 polypeptide having an amino acid sequence having at least at least 70%, at least 75%, at least 80%, at least 85%, at least 90%, at least 95%, at least 98%, at least 99%, or 100%, amino acid sequence identity to the amino acid sequence of the HLA-A*2402 polypeptide depicted in FIG. 20F. In some cases, a TMMP comprises an IgG1 Fc polypeptide having an amino acid sequence having at least at least 70%, at least 75%, at least 80%, at least 85%, at least 90%, at least 95%, at least 98%, at least 99%, or 100%, amino acid sequence identity to the amino acid sequence of the IgG1 Fc polypeptide depicted in FIG. 20F. In some cases, a TMMP comprises an IL-2 polypeptide having an amino acid sequence having at least at least 70%, at least 75%, at least 80%, at least 85%, at least 90%, at least 95%, at least 98%, at least 99%, or 100%, amino acid sequence identity to the amino acid sequence of the IL-2 polypeptide depicted in FIG. 20F.

In some cases, a TMMP comprises an HLA-E*0101 polypeptide having an amino acid sequence having at least at least 70%, at least 75%, at least 80%, at least 85%, at least 90%, at least 95%, at least 98%, at least 99%, or 100%, amino acid sequence identity to the amino acid sequence of the HLA-E*0101 polypeptide depicted in FIG. 20H. In some cases, a TMMP comprises an IgG1 Fc polypeptide having an amino acid sequence having at least at least 70%, at least 75%, at least 80%, at least 85%, at least 90%, at least 95%, at least 98%, at least 99%, or 100%, amino acid sequence identity to the amino acid sequence of the IgG1 Fc polypeptide depicted in FIG. 20H. In some cases, a TMMP comprises an IL-2 polypeptide having an amino acid sequence having at least at least 70%, at least 75%, at least 80%, at least 85%, at least 90%, at least 95%, at least 98%, at least 99%, or 100%, amino acid sequence identity to the amino acid sequence of the IL-2 polypeptide depicted in FIG. 20H.

In some cases, a TMMP comprises an HLA-E*0103 polypeptide having an amino acid sequence having at least at least 70%, at least 75%, at least 80%, at least 85%, at least 90%, at least 95%, at least 98%, at least 99%, or 100%, amino acid sequence identity to the amino acid sequence of the HLA-E*0103 polypeptide depicted in FIG. 20J. In some cases, a TMMP comprises an IgG1 Fc polypeptide having an amino acid sequence having at least at least 70%, at least 75%, at least 80%, at least 85%, at least 90%, at least 95%, at least 98%, at least 99%, or 100%, amino acid sequence identity to the amino acid sequence of the IgG1 Fc polypeptide depicted in FIG. 20J. In some cases, a TMMP comprises an IL-2 polypeptide having an amino acid sequence having at least at least 70%, at least 75%, at least 80%, at least 85%, at least 90%, at least 95%, at least 98%, at least 99%, or 100%, amino acid sequence identity to the amino acid sequence of the IL-2 polypeptide depicted in FIG. 20J.

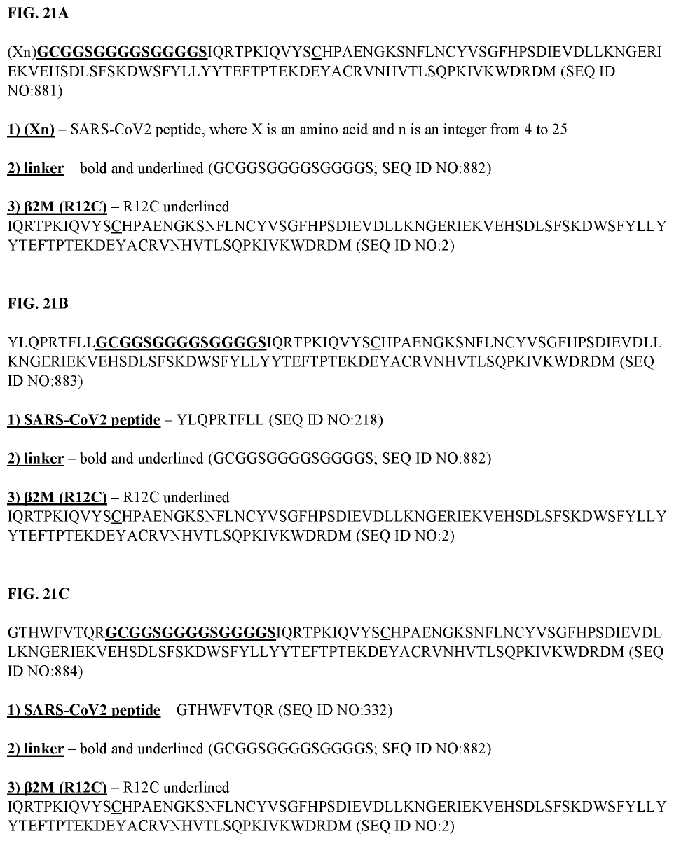

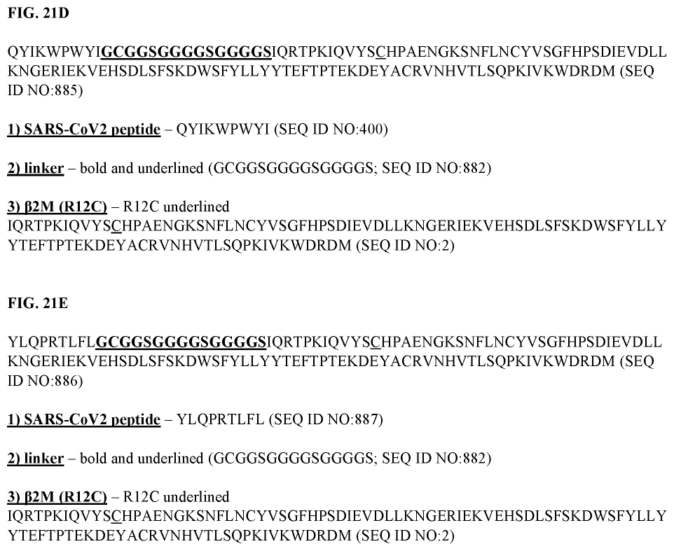

As one non-limiting example, a TMMP comprises: a) a first polypeptide comprising an amino acid sequence having at least 90%, at least 95%, at least 98%, or at least 99%, amino acid sequence identity to the amino acid sequence depicted in FIG. 20A; and b) a second polypeptide comprising an amino acid sequence having at least 90%, at least 95%, at least 98%, or at least 99%, amino acid sequence identity to the amino acid sequence depicted in FIG. 21A. As one non-limiting example, a TMMP comprises: a) a first polypeptide comprising the amino acid sequence depicted in FIG. 20A; and b) a second polypeptide comprising the amino acid sequence depicted in FIG. 21A. In some cases, (Xn) is a SARS-CoV-2 peptide having one of the amino acid sequences depicted in FIG. 19. In some cases, the SARS-CoV-2 peptide comprises the amino acid sequence GTHWFVTQR (SEQ ID NO:332) and has a length of 9 amino acids. In some cases, the SARS-CoV-2 peptide comprises the amino acid sequence QYIKWPWYI (SEQ ID NO:400) and has a length of 9 amino acids.

In some cases, a TMMP comprises: a) a first polypeptide comprising the amino acid sequence depicted in FIG. 20A, except that instead of an anti-CD19 scFv, the first polypeptide comprises an anti-Her2 scFv; and b) a second polypeptide comprising the amino acid sequence depicted in FIG. 21A. In some cases, (Xn) is a SARS-CoV-2 peptide having one of the amino acid sequences depicted in FIG. 19. In some cases, the SARS-CoV-2 peptide comprises the amino acid sequence GTHWFVTQR (SEQ ID NO:332) and has a length of 9 amino acids. In some cases, the SARS-CoV-2 peptide comprises the amino acid sequence QYIKWPWYI (SEQ ID NO:400) and has a length of 9 amino acids.

In some cases, a TMMP comprises: a) a first polypeptide comprising the amino acid sequence depicted in FIG. 20A, except that instead of an anti-CD19 scFv, the first polypeptide comprises an anti-CD22 scFv; and b) a second polypeptide comprising the amino acid sequence depicted in FIG. 21A. In some cases, (Xn) is a SARS-CoV-2 peptide having one of the amino acid sequences depicted in FIG. 19. In some cases, the SARS-CoV-2 peptide comprises the amino acid sequence GTHWFVTQR (SEQ ID NO:332) and has a length of 9 amino acids. In some cases, the SARS-CoV-2 peptide comprises the amino acid sequence QYIKWPWYI (SEQ ID NO:400) and has a length of 9 amino acids.

In some cases, a TMMP comprises: a) a first polypeptide comprising the amino acid sequence depicted in FIG. 20A, except that instead of an anti-CD19 scFv, the first polypeptide comprises an anti-TROP-2 scFv; and b) a second polypeptide comprising the amino acid sequence depicted in FIG. 21A. In some cases, (Xn) is a SARS-CoV-2 peptide having one of the amino acid sequences depicted in FIG. 19. In some cases, the SARS-CoV-2 peptide comprises the amino acid sequence GTHWFVTQR (SEQ ID NO:332) and has a length of 9 amino acids. In some cases, the SARS-CoV-2 peptide comprises the amino acid sequence QYIKWPWYI (SEQ ID NO:400) and has a length of 9 amino acids.

In some cases, a TMMP comprises: a) a first polypeptide comprising the amino acid sequence depicted in FIG. 20A, except that instead of an anti-CD19 scFv, the first polypeptide comprises an anti-mesothelin scFv; and b) a second polypeptide comprising the amino acid sequence depicted in FIG. 21A. In some cases, (Xn) is a SARS-CoV-2 peptide having one of the amino acid sequences depicted in FIG. 19. In some cases, the SARS-CoV-2 peptide comprises the amino acid sequence GTHWFVTQR (SEQ ID NO:332) and has a length of 9 amino acids. In some cases, the SARS-CoV-2 peptide comprises the amino acid sequence QYIKWPWYI (SEQ ID NO:400) and has a length of 9 amino acids.

In some cases, a TMMP comprises: a) a first polypeptide comprising the amino acid sequence depicted in FIG. 20A, except that instead of an anti-CD19 scFv, the first polypeptide comprises an anti-BCMA scFv; and b) a second polypeptide comprising the amino acid sequence depicted in FIG. 21A. In some cases, (Xn) is a SARS-CoV-2 peptide having one of the amino acid sequences depicted in FIG. 19. In some cases, the SARS-CoV-2 peptide comprises the amino acid sequence GTHWFVTQR (SEQ ID NO:332) and has a length of 9 amino acids. In some cases, the SARS-CoV-2 peptide comprises the amino acid sequence QYIKWPWYI (SEQ ID NO:400) and has a length of 9 amino acids.

As one non-limiting example, a TMMP comprises: a) a first polypeptide comprising an amino acid sequence having at least 90%, at least 95%, at least 98%, or at least 99%, amino acid sequence identity to the amino acid sequence depicted in FIG. 20A; and b) a second polypeptide comprising an amino acid sequence having at least 90%, at least 95%, at least 98%, or at least 99%, amino acid sequence identity to the amino acid sequence depicted in FIG. 21B. In some cases, a TMMP comprises: a) a first polypeptide comprising the amino acid sequence depicted in FIG. 20A; and b) a second polypeptide comprising the amino acid sequence depicted in FIG. 21B.

As one non-limiting example, a TMMP comprises: a) a first polypeptide comprising an amino acid sequence having at least 90%, at least 95%, at least 98%, or at least 99%, amino acid sequence identity to the amino acid sequence depicted in FIG. 20A; and b) a second polypeptide comprising an amino acid sequence having at least 90%, at least 95%, at least 98%, or at least 99%, amino acid sequence identity to the amino acid sequence depicted in FIG. 21C. In some cases, a TMMP comprises: a) a first polypeptide comprising the amino acid sequence depicted in FIG. 20A; and b) a second polypeptide comprising the amino acid sequence depicted in FIG. 21C.

As one non-limiting example, a TMMP comprises: a) a first polypeptide comprising an amino acid sequence having at least 90%, at least 95%, at least 98%, or at least 99%, amino acid sequence identity to the amino acid sequence depicted in FIG. 20A; and b) a second polypeptide comprising an amino acid sequence having at least 90%, at least 95%, at least 98%, or at least 99%, amino acid sequence identity to the amino acid sequence depicted in FIG. 21D. In some cases, a TMMP comprises: a) a first polypeptide comprising the amino acid sequence depicted in FIG. 20A; and b) a second polypeptide comprising the amino acid sequence depicted in FIG. 21D.

As one non-limiting example, a TMMP comprises: a) a first polypeptide comprising an amino acid sequence having at least 90%, at least 95%, at least 98%, or at least 99%, amino acid sequence identity to the amino acid sequence depicted in FIG. 20E; and b) a second polypeptide comprising an amino acid sequence having at least 90%, at least 95%, at least 98%, or at least 99%, amino acid sequence identity to the amino acid sequence depicted in FIG. 21B. In some cases, a TMMP comprises: a) a first polypeptide comprising the amino acid sequence depicted in FIG. 20E; and b) a second polypeptide comprising the amino acid sequence depicted in FIG. 21B.

As one non-limiting example, a TMMP comprises: a) a first polypeptide comprising an amino acid sequence having at least 90%, at least 95%, at least 98%, or at least 99%, amino acid sequence identity to the amino acid sequence depicted in FIG. 20D; and b) a second polypeptide comprising an amino acid sequence having at least 90%, at least 95%, at least 98%, or at least 99%, amino acid sequence identity to the amino acid sequence depicted in FIG. 21C. In some cases, a TMMP comprises: a) a first polypeptide comprising the amino acid sequence depicted in FIG. 20C; and b) a second polypeptide comprising the amino acid sequence depicted in FIG. 21C.

As one non-limiting example, a TMMP comprises: a) a first polypeptide comprising an amino acid sequence having at least 90%, at least 95%, at least 98%, or at least 99%, amino acid sequence identity to the amino acid sequence depicted in FIG. 20E; and b) a second polypeptide comprising an amino acid sequence having at least 90%, at least 95%, at least 98%, or at least 99%, amino acid sequence identity to the amino acid sequence depicted in FIG. 21D. In some cases, a TMMP comprises: a) a first polypeptide comprising the amino acid sequence depicted in FIG. 20E; and b) a second polypeptide comprising the amino acid sequence depicted in FIG. 21D.

As noted above, in some cases, a TMMP comprises an HLA-E heavy chain polypeptide. In some cases, the TMMP comprises a SARS-CoV-2 peptide having the amino acid sequence YLQPRTLFL (SEQ ID NO:887). In some cases, the TMMP comprises a SARS-CoV-2 peptide having the amino acid sequence YLQPRTLIL (SEQ ID NO:1201). In some cases, the TMMP comprises a SARS-CoV-2 peptide having the amino acid sequence YLQPRTLVL (SEQ ID NO:1202). As one non-limiting example, a TMMP comprises: a) a first polypeptide comprising the amino acid sequence depicted in FIG. 20G; and b) a second polypeptide comprising the amino acid sequence depicted in FIG. 21E. As another non-limiting example, a TMMP comprises: a) a first polypeptide comprising the amino acid sequence depicted in FIG. 20I; and b) a second polypeptide comprising the amino acid sequence depicted in FIG. 21E. In some cases, the TMMP has a TTP other than an anti-CD19 scFv. For example, in some cases, a TMMP comprises: a) a first polypeptide comprising the amino acid sequence depicted in FIG. 20G, except that instead of an anti-CD19 scFv, the first polypeptide comprises an anti-mesothelin scFv, an anti-CD22 scFv, or any other anti-cancer-associated scFv or nanobody; and b) a second polypeptide comprising the amino acid sequence depicted in FIG. 21E. As another example, in some cases, a TMMP comprises: a) a first polypeptide comprising the amino acid sequence depicted in FIG. 20I, except that instead of an anti-CD19 scFv, the first polypeptide comprises an anti-mesothelin scFv; and b) a second polypeptide comprising the amino acid sequence depicted in FIG. 21E.

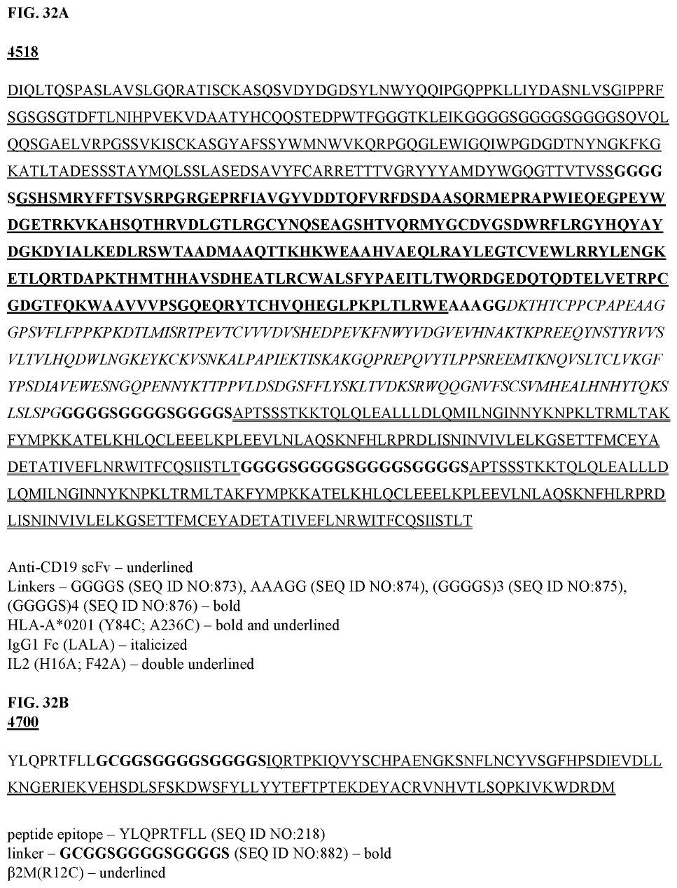

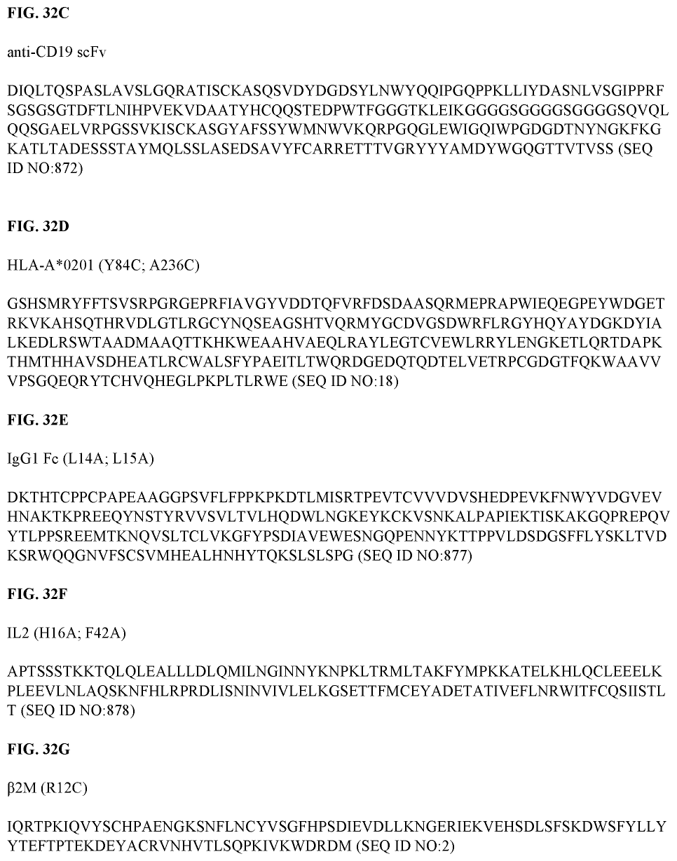

As one non-limiting example, a TMMP comprises: a) a first polypeptide comprising: i) a SARS-CoV2 peptide that has the amino acid sequence YLQPRTFLL (SEQ ID NO:218); ii) a linker comprising the amino acid sequence GCGGS(GGGS)2 (SEQ ID NO:1203); and iii) a β2M polypeptide comprising an amino acid sequence having at least 95%, at least 98%, at least 99%, or 100%, amino acid sequence identity to the following β2M amino acid sequence: IQRTPKIQVYSCHPAENGKSNFLNCYVSGFHPSDIEVDLLKNGERIEKVEHSDLSFSKDWSFYLL YYTEFTPTEKDEYACRVNHVTLSQPKIVKWDRDM (SEQ ID NO:2), where amino acid 12 is a Cys; and b) a second polypeptide comprising: i) an anti-CD19 scFv comprising an amino acid sequence having at least 95%, at least 98%, at least 99%, or 100%, amino acid sequence identity to the following anti-CD19 scFv amino acid sequence: DIQLTQSPASLAVSLGQRATISCKASQSVDYDGDSYLNWYQQIPGQPPKLLIYDASNLVSGIPPRF SGSGSGTDFTLNIHPVEKVDAATYHCQQSTEDPWTFGGGTKLEIKGGGGSGGGGSGGGGSQVQ LQQSGAELVRPGSSVKISCKASGYAFSSYWMNWVKQRPGQGLEWIGQIWPGDGDTNYNGKFK GKATLTADESSSTAYMQLSSLASEDSAVYFCARRETTTVGRYYYAMDYWGQGTTVTVSS (SEQ ID NO:872); ii) a linker, e.g., where the linker has the sequence GGGGS (SEQ ID NO:873); iii) an HLA-A*0201 polypeptide comprising an amino acid sequence having at least 95%, at least 98%, at least 99%, or 100%, amino acid sequence identity to the following HLA-A*0201 amino acid sequence: GSHSMRYFFTSVSRPGRGEPRFIAVGYVDDTQFVRFDSDAASQRMEPRAPWIEQEGPEYWDGET RKVKAHSQTHRVDLGTLRGCYNQSEAGSHTVQRMYGCDVGSDWRFLRGYHQYAYDGKDYIA LKEDLRSWTAADMAAQTTKHKWEAAHVAEQLRAYLEGTCVEWLRRYLENGKETLQRTDAPK THMTHHAVSDHEATLRCWALSFYPAEITLTWQRDGEDQTQDTELVETRPCGDGTFQKWAAVV VPSGQEQRYTCHVQHEGLPKPLTLRWE (SEQ ID NO:18), where amino acid 84 is a Cys and where amino acid 236 is a Cys; iv) a linker, e.g., where the linker has the sequence AAAGG (SEQ ID NO:874); v) an IgG Fc polypeptide comprising an amino acid sequence having at least 95%, at least 98%, at least 99%, or 100%, amino acid sequence identity to the following IgG Fc amino acid sequence: DKTHTCPPCPAPEAAGGPSVFLFPPKPKDTLMISRTPEVTCVVVDVSHEDPEVKFNWYVDGVEV HNAKTKPREEQYNSTYRVVSVLTVLHQDWLNGKEYKCKVSNKALPAPIEKTISKAKGQPREPQ VYTLPPSREEMTKNQVSLTCLVKGFYPSDIAVEWESNGQPENNYKTTPPVLDSDGSFFLYSKLT VDKSRWQQGNVFSCSVMHEALHNHYTQKSLSLSPG (SEQ ID NO:877), where amino acid 14 is an Ala and where amino acid 15 is an Ala; vi) a linker, e.g., where the linker has the amino acid sequence (GGGGS)3 (SEQ ID NO:875); vii) a first MOD, where the first MOD comprises an amino acid sequence having at least 95%, at least 98%, at least 99%, or 100%, amino acid sequence identity to the following variant IL-2 amino acid sequence: APTSSSTKKTQLQLEALLLDLQMILNGINNYKNPKLTRMLTAKFYMPKKATELKHLQCLEEELK PLEEVLNLAQSKNFHLRPRDLISNINVIVLELKGSETTFMCEYADETATIVEFLNRWITFCQSIIST LT (SEQ ID NO:878), e.g., where amino acid 16 is an Ala and where amino acid 42 is an Ala; viii) a linker, e.g., where the linker has the amino acid sequence (GGGGS)4 (SEQ ID NO:876); and ix) a second MOD, where the second MOD comprises an amino acid sequence having at least 95%, at least 98%, at least 99%, or 100%, amino acid sequence identity to the following variant IL-2 amino acid sequence: APTSSSTKKTQLQLEALLLDLQMILNGINNYKNPKLTRMLTAKFYMPKKATELKHLQCLEEELK PLEEVLNLAQSKNFHLRPRDLISNINVIVLELKGSETTFMCEYADETATIVEFLNRWITFCQSIIST LT (SEQ ID NO:878), e.g., where amino acid 16 is an Ala and where amino acid 42 is an Ala. As an example, in some cases, a TMMP comprises a) a first polypeptide comprising an amino acid sequence having at least 95%, at least 98%, at least 99%, or 100%, amino acid sequence identity to the amino acid sequence of the “4700” construct depicted in FIG. 32B; and b) a second polypeptide comprising an amino acid sequence having at least 95%, at least 98%, at least 99%, or 100%, amino acid sequence identity to the amino acid sequence of the “4518” construct depicted in FIG. 32A. In some cases, a TMMP comprises: a) a first polypeptide having the amino acid sequence of the “4700” construct depicted in FIG. 32B; and b) a second polypeptide having the amino acid sequence of the “4518” construct depicted in FIG. 32A.

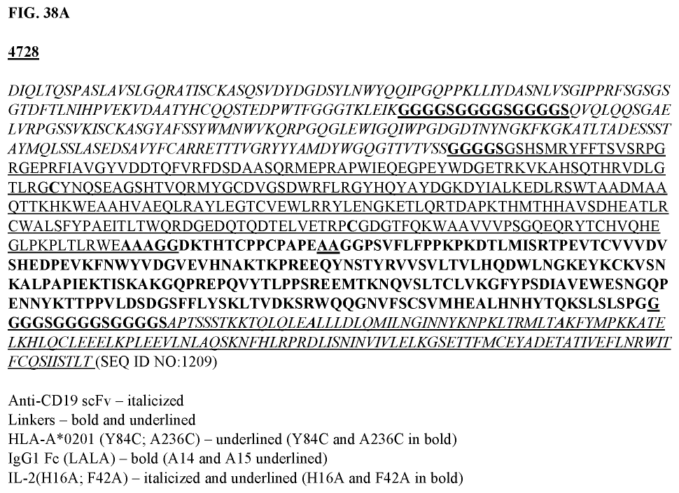

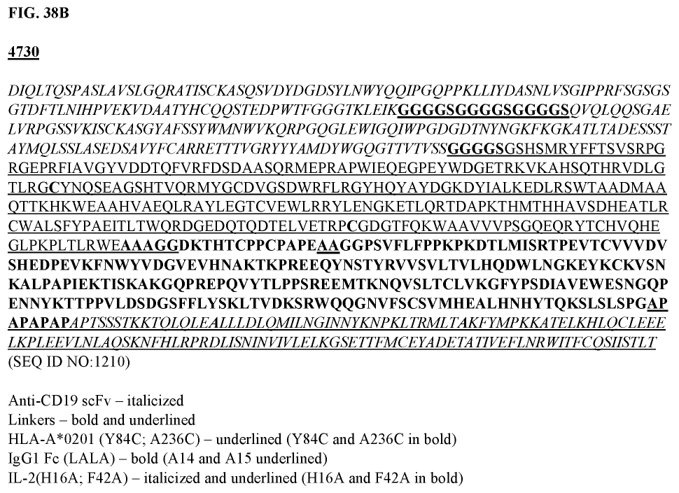

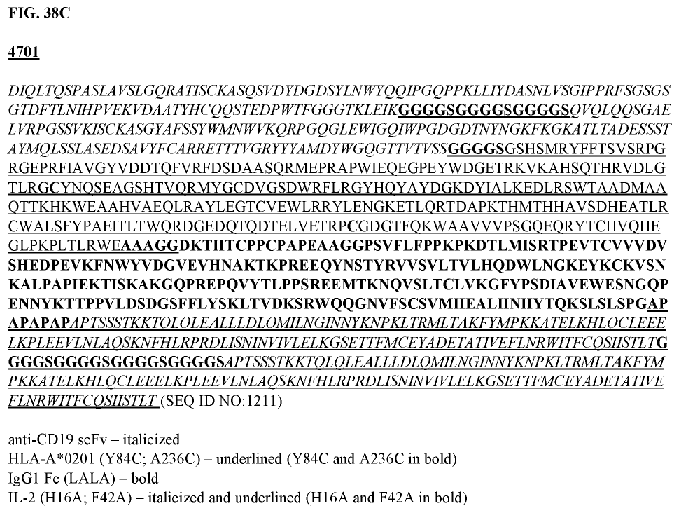

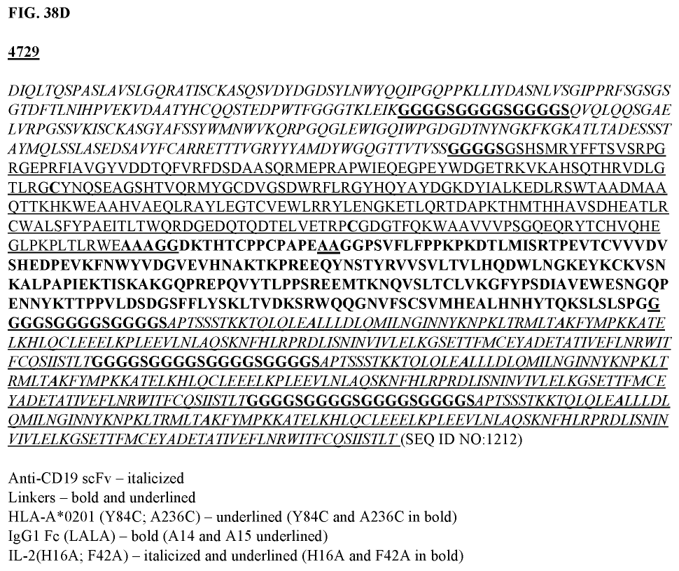

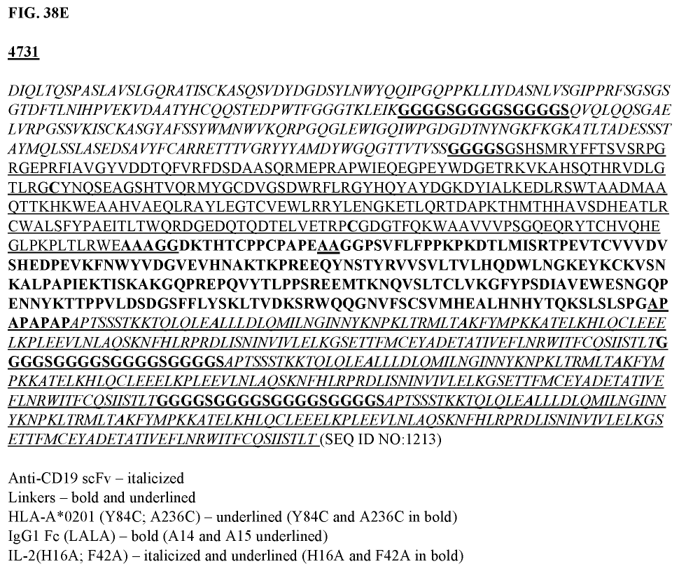

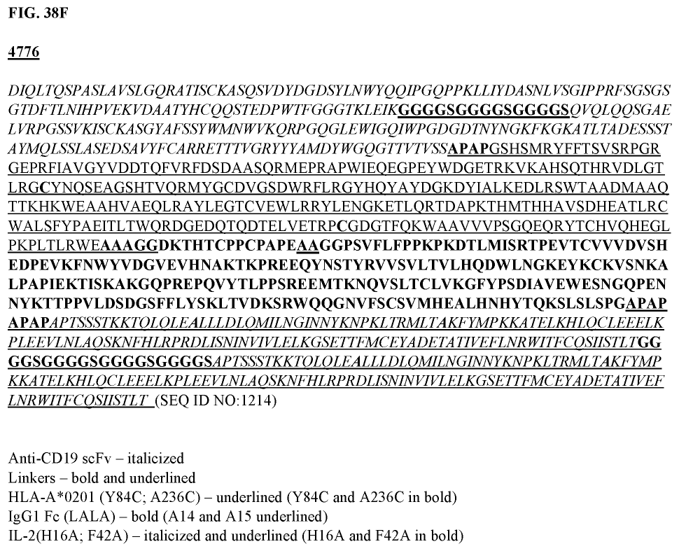

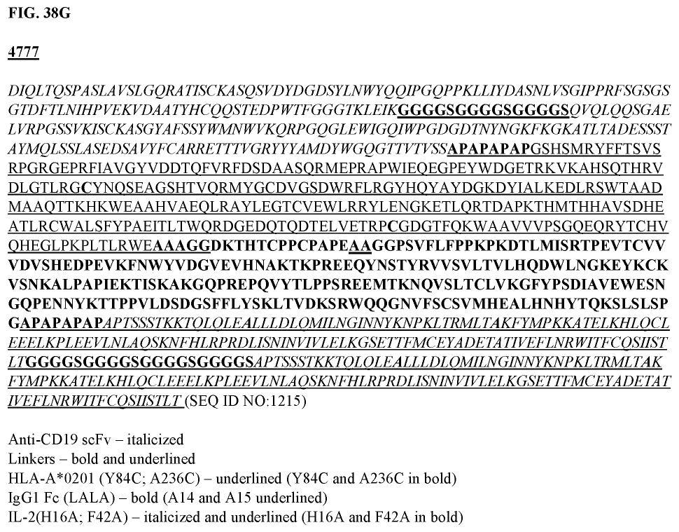

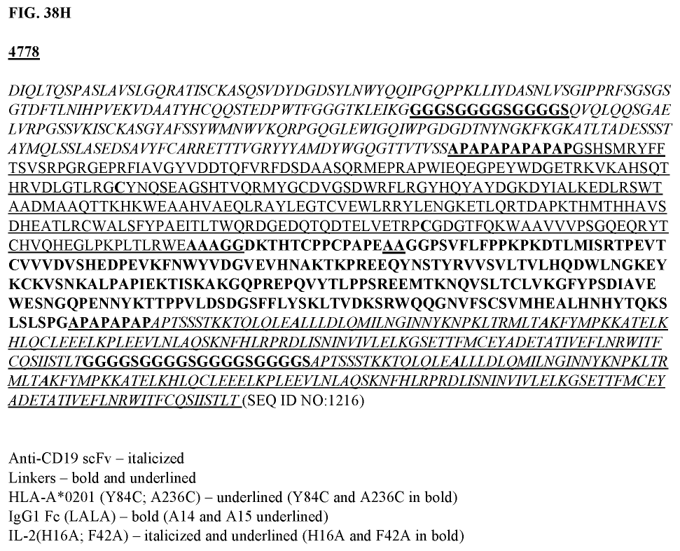

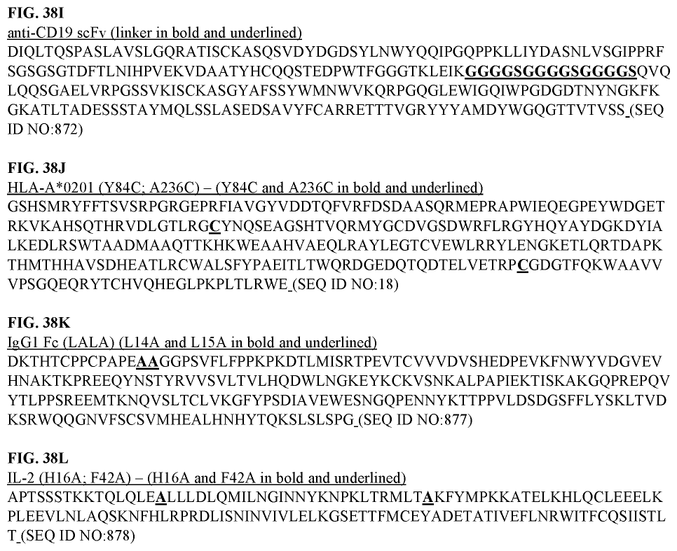

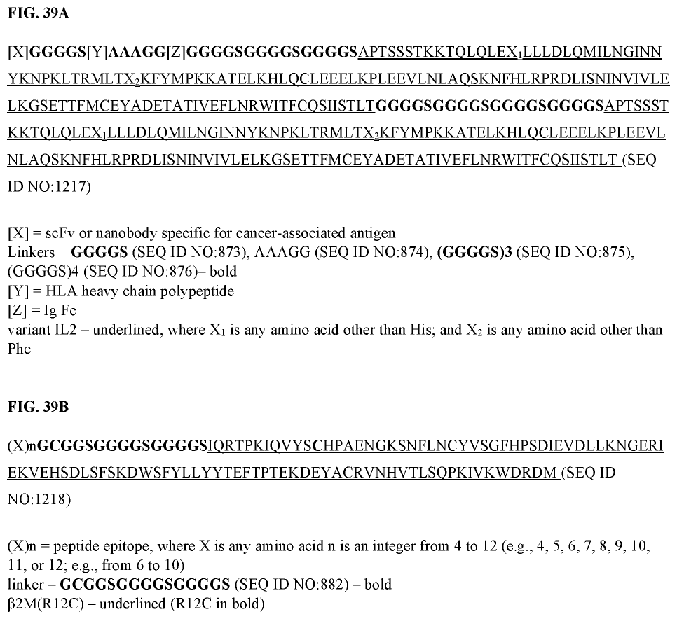

In some cases, a TMMP comprises: a) a first polypeptide comprising an amino acid sequence having at least 95%, at least 98%, at least 99%, or 100%, amino acid sequence identity to the amino acid sequence of the “4700” construct depicted in FIG. 32B; and b) a second polypeptide comprising an amino acid sequence having at least 95%, at least 98%, at least 99%, or 100%, amino acid sequence identity to the amino acid sequence of the “4728” construct depicted in FIG. 38A. In some cases, a TMMP comprises: a) a first polypeptide having the amino acid sequence of the “4700” construct depicted in FIG. 32B; and b) a second polypeptide having the amino acid sequence of the “4728” construct depicted in FIG. 38A.

In some cases, a TMMP comprises: a) a first polypeptide comprising an amino acid sequence having at least 95%, at least 98%, at least 99%, or 100%, amino acid sequence identity to the amino acid sequence of the “4700” construct depicted in FIG. 32B; and b) a second polypeptide comprising an amino acid sequence having at least 95%, at least 98%, at least 99%, or 100%, amino acid sequence identity to the amino acid sequence of the “4730” construct depicted in FIG. 38B. In some cases, a TMMP comprises: a) a first polypeptide having the amino acid sequence of the “4700” construct depicted in FIG. 32B; and b) a second polypeptide having the amino acid sequence of the “4730” construct depicted in FIG. 38B.

In some cases, a TMMP comprises: a) a first polypeptide comprising an amino acid sequence having at least 95%, at least 98%, at least 99%, or 100%, amino acid sequence identity to the amino acid sequence of the “4700” construct depicted in FIG. 32B; and b) a second polypeptide comprising an amino acid sequence having at least 95%, at least 98%, at least 99%, or 100%, amino acid sequence identity to the amino acid sequence of the “4701” construct depicted in FIG. 38C. In some cases, a TMMP comprises: a) a first polypeptide having the amino acid sequence of the “4700” construct depicted in FIG. 32B; and b) a second polypeptide having the amino acid sequence of the “4701” construct depicted in FIG. 38C.

In some cases, a TMMP comprises: a) a first polypeptide comprising an amino acid sequence having at least 95%, at least 98%, at least 99%, or 100%, amino acid sequence identity to the amino acid sequence of the “4700” construct depicted in FIG. 32B; and b) a second polypeptide comprising an amino acid sequence having at least 95%, at least 98%, at least 99%, or 100%, amino acid sequence identity to the amino acid sequence of the “4729” construct depicted in FIG. 38D. In some cases, a TMMP comprises: a) a first polypeptide having the amino acid sequence of the “4700” construct depicted in FIG. 32B; and b) a second polypeptide having the amino acid sequence of the “4729” construct depicted in FIG. 38D.

In some cases, a TMMP comprises: a) a first polypeptide comprising an amino acid sequence having at least 95%, at least 98%, at least 99%, or 100%, amino acid sequence identity to the amino acid sequence of the “4700” construct depicted in FIG. 32B; and b) a second polypeptide comprising an amino acid sequence having at least 95%, at least 98%, at least 99%, or 100%, amino acid sequence identity to the amino acid sequence of the “4731” construct depicted in FIG. 38E. In some cases, a TMMP comprises: a) a first polypeptide having the amino acid sequence of the “4700” construct depicted in FIG. 32B; and b) a second polypeptide having the amino acid sequence of the “4731” construct depicted in FIG. 38E.

In some cases, a TMMP comprises: a) a first polypeptide comprising an amino acid sequence having at least 95%, at least 98%, at least 99%, or 100%, amino acid sequence identity to the amino acid sequence of the “4700” construct depicted in FIG. 32B; and b) a second polypeptide comprising an amino acid sequence having at least 95%, at least 98%, at least 99%, or 100%, amino acid sequence identity to the amino acid sequence of the “4776” construct depicted in FIG. 38F. In some cases, a TMMP comprises: a) a first polypeptide having the amino acid sequence of the “4700” construct depicted in FIG. 32B; and b) a second polypeptide having the amino acid sequence of the “4776” construct depicted in FIG. 38F.

In some cases, a TMMP comprises: a) a first polypeptide comprising an amino acid sequence having at least 95%, at least 98%, at least 99%, or 100%, amino acid sequence identity to the amino acid sequence of the “4700” construct depicted in FIG. 32B; and b) a second polypeptide comprising an amino acid sequence having at least 95%, at least 98%, at least 99%, or 100%, amino acid sequence identity to the amino acid sequence of the “4777” construct depicted in FIG. 38G. In some cases, a TMMP comprises: a) a first polypeptide having the amino acid sequence of the “4700” construct depicted in FIG. 32B; and b) a second polypeptide having the amino acid sequence of the “4777” construct depicted in FIG. 38G.

In some cases, a TMMP comprises: a) a first polypeptide comprising an amino acid sequence having at least 95%, at least 98%, at least 99%, or 100%, amino acid sequence identity to the amino acid sequence of the “4700” construct depicted in FIG. 32B; and b) a second polypeptide comprising an amino acid sequence having at least 95%, at least 98%, at least 99%, or 100%, amino acid sequence identity to the amino acid sequence of the “4778” construct depicted in FIG. 38H. In some cases, a TMMP comprises: a) a first polypeptide having the amino acid sequence of the “4700” construct depicted in FIG. 32B; and b) a second polypeptide having the amino acid sequence of the “4778” construct depicted in FIG. 38H.

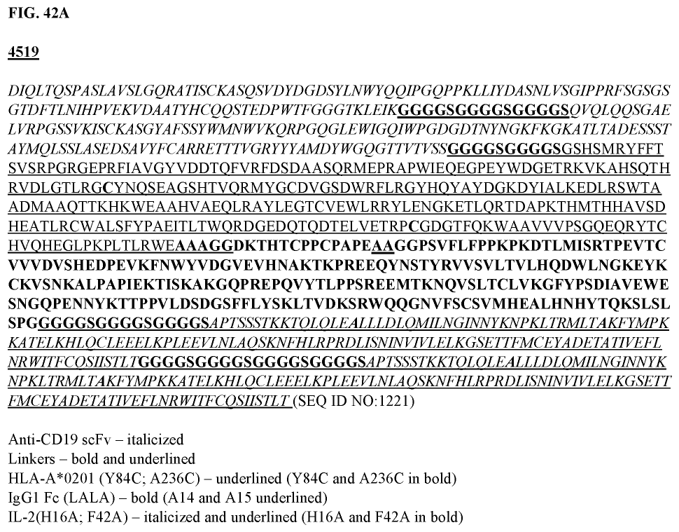

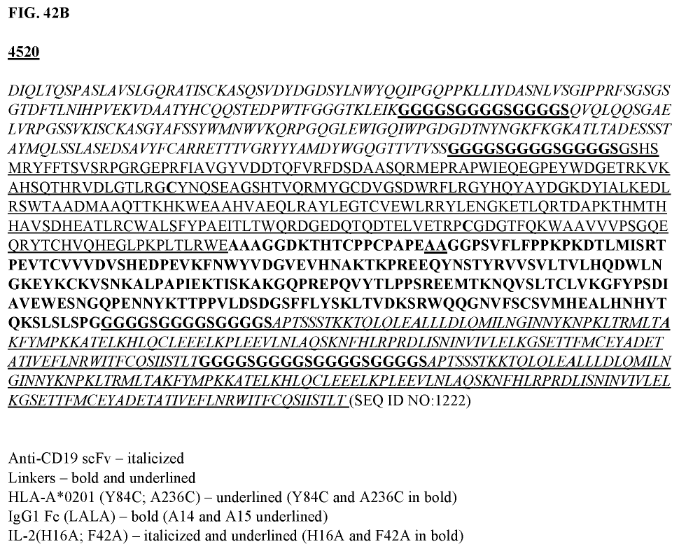

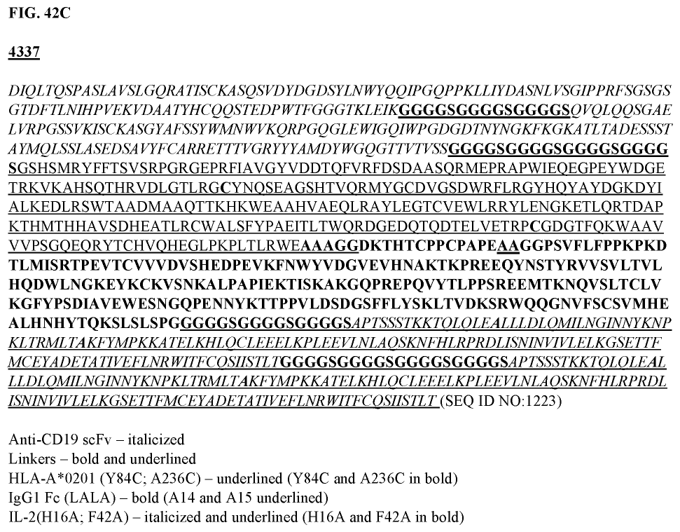

In some cases, a TMMP comprises: a) a first polypeptide comprising an amino acid sequence having at least 95%, at least 98%, at least 99%, or 100%, amino acid sequence identity to the amino acid sequence of the “4700” construct depicted in FIG. 32B; and b) a second polypeptide comprising an amino acid sequence having at least 95%, at least 98%, at least 99%, or 100%, amino acid sequence identity to the amino acid sequence of the “4519” construct depicted in FIG. 42A. In some cases, a TMMP comprises: a) a first polypeptide having the amino acid sequence of the “4700” construct depicted in FIG. 32B; and b) a second polypeptide having the amino acid sequence of the “4519” construct depicted in FIG. 42A.

In some cases, a TMMP comprises: a) a first polypeptide comprising an amino acid sequence having at least 95%, at least 98%, at least 99%, or 100%, amino acid sequence identity to the amino acid sequence of the “4700” construct depicted in FIG. 32B; and b) a second polypeptide comprising an amino acid sequence having at least 95%, at least 98%, at least 99%, or 100%, amino acid sequence identity to the amino acid sequence of the “4520” construct depicted in FIG. 42B. In some cases, a TMMP comprises: a) a first polypeptide having the amino acid sequence of the “4700” construct depicted in FIG. 32B; and b) a second polypeptide having the amino acid sequence of the “4520” construct depicted in FIG. 42B.

In some cases, a TMMP comprises: a) a first polypeptide comprising an amino acid sequence having at least 95%, at least 98%, at least 99%, or 100%, amino acid sequence identity to the amino acid sequence of the “4700” construct depicted in FIG. 32B; and b) a second polypeptide comprising an amino acid sequence having at least 95%, at least 98%, at least 99%, or 100%, amino acid sequence identity to the amino acid sequence of the “4337” construct depicted in FIG. 42C. In some cases, a TMMP comprises: a) a first polypeptide having the amino acid sequence of the “4700” construct depicted in FIG. 32B; and b) a second polypeptide having the amino acid sequence of the “4337” construct depicted in FIG. 42C.

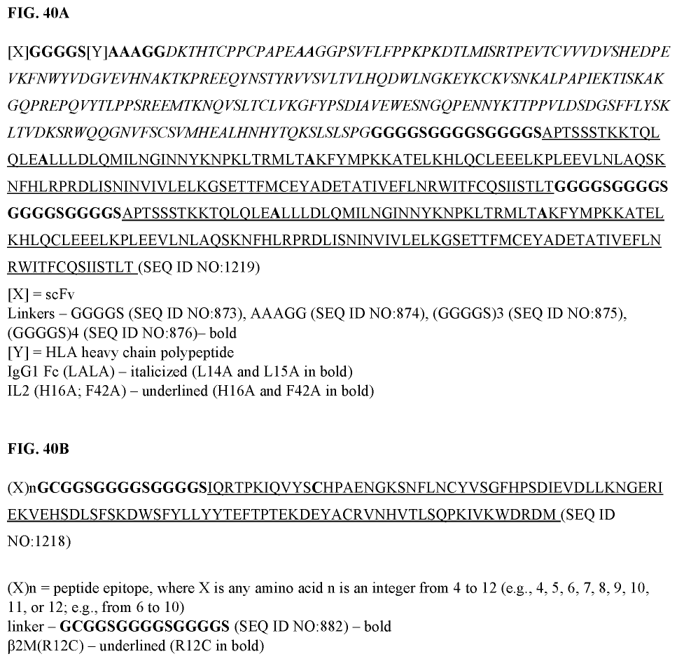

In some cases, a TMMP comprises: a)) a first polypeptide comprising: i) a peptide epitope having a length of from 4 amino acids to 25 amino acids (e.g., from 6 to 12 amino acids; from 8 to 12 amino acids; etc.); ii) a linker having the amino acid sequence GCGGS(GGGGS)2 (SEQ ID NO:882); and iii) a β2M polypeptide comprising an amino acid sequence having at least 95%, at least 98%, at least 99%, or 100%, amino acid sequence identity to the β2M amino acid sequence depicted in FIG. 32G; and b) a second polypeptide of the structure: [X]-GGGGS-[Y]-AAAGG-[Z]-(GGGGS)3-APTSSSTKKTQLQLEX₁LLLDLQMILNGINNYKNPKLTRMLTX₂KFYMPKKATELKHLQCLEEEL KPLEEVLNLAQSKNFHLRPRDLISNINVIVLELKGSETTFMCEYADETATIVEFLNRWITFCQSIIS TLT-(GGGGS)4-APTSSSTKKTQLQLEX₁LLLDLQMILNGINNYKNPKLTRMLTX₂KFYMPKKATELKHLQCLEEEL KPLEEVLNLAQSKNFHLRPRDLISNINVIVLELKGSETTFMCEYADETATIVEFLNRWITFCQSIIS TLT (SEQ ID NO:1217), where [X] is a scFv specific for a cancer-associated antigen or a nanobody specific for a cancer-associated antigen; where [Y] is an HLA heavy chain polypeptide; where [Z] is an Ig Fc polypeptide; where X₁ is an amino acid other than His; and where X₂ is an amino acid other than Phe. Such a TMMP is depicted in FIG. 40A and FIG. 40B. In some cases, the Ig Fc polypeptide is a variant of a Fc polypeptide such as a human IgG1 Fc, which variant has a substantially reduced ability to effect CDC or ADCC.

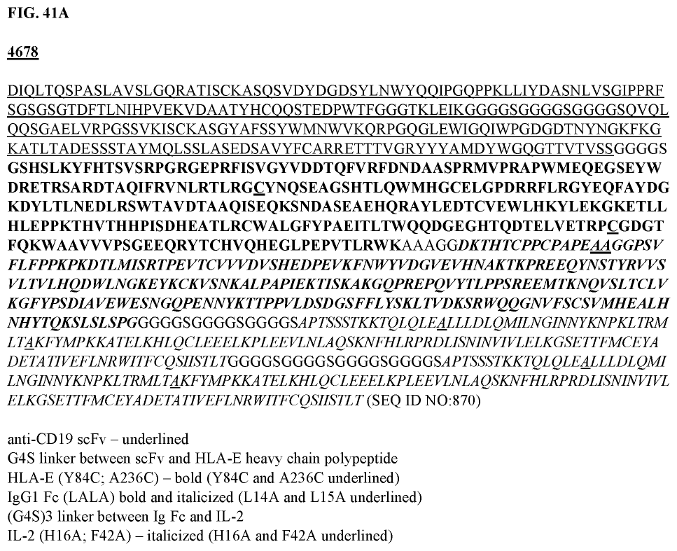

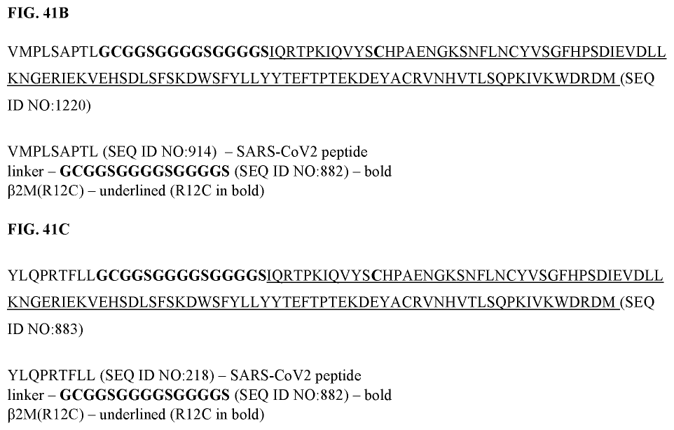

In some cases, a TMMP comprises: a) a first polypeptide comprising: i) a peptide epitope having a length of from 4 amino acids to 25 amino acids (e.g., from 6 to 12 amino acids; from 8 to 12 amino acids; etc.); ii) a linker having the amino acid sequence GCGGS(GGGGS)2 (SEQ ID NO:882); and iii) a β2M polypeptide comprising an amino acid sequence having at least 95%, at least 98%, at least 99%, or 100%, amino acid sequence identity to the β2M amino acid sequence depicted in FIG. 32G; and b) a second polypeptide comprising: i) a scFv polypeptide or a nanobody specific for a cancer-associated antigen; ii) a linker having the sequence GGGGS (SEQ ID NO:873); iii) an HLA heavy chain polypeptide; iv) a linker having the sequence AAAGG (SEQ ID NO:874); v) an Ig Fc polypeptide comprising an amino acid sequence having at least 95%, at least 98%, at least 99%, or 100%, amino acid sequence identity to the Ig Fc amino acid sequence depicted in FIG. 38K; vi) a linker having the sequence (GGGGS)3 (SEQ ID NO:875); vii) a variant IL-2 polypeptide comprising an amino acid sequence having at least 95%, at least 98%, at least 99%, or 100%, amino acid sequence identity to the variant IL-2 amino acid sequence depicted in FIG. 38L, where amino acid 16 is other than His (e.g., where amino acid 16 is Ala) and where amino acid 42 is other than Phe (e.g., where amino acid 42 is Ala); viii) a linker having the sequence (GGGGS)4 (SEQ ID NO:876); and ix) a variant IL-2 polypeptide comprising an amino acid sequence having at least 95%, at least 98%, at least 99%, or 100%, amino acid sequence identity to the variant IL-2 amino acid sequence depicted in FIG. 38L, where amino acid 16 is other than His (e.g., where amino acid 16 is Ala) and where amino acid 42 is other than Phe (e.g., where amino acid 42 is Ala). Such a TMMP is depicted in FIG. 40A and FIG. 40B. In some cases, the Ig Fc polypeptide is a variant of a Fc polypeptide such as a human IgG1 Fc, which variant has a substantially reduced ability to effect CDC or ADCC; for example, in some cases, the variant Ig Fc polypeptide comprises an Ala (instead of a Leu) at position 14 and an Ala (instead of a Leu) at position 15, based on the amino acid numbering set out in FIG. 38K. In some cases, the peptide epitope is a SARS-CoV-2 peptide. In some cases, the HLA heavy chain polypeptide is an HLA-A*0201 polypeptide; e.g., an HLA polypeptide comprising an amino acid sequence having at least 95%, at least 98%, at least 99%, or 100%, amino acid sequence identity to the amino acid sequence depicted in FIG. 3A. In some cases, the HLA heavy chain polypeptide is an HLA-A*0201 polypeptide comprising Y84C and A236C substitutions (e.g., as depicted in FIG. 3C). In some cases, the HLA heavy chain polypeptide is an HLA-A*1101 polypeptide; e.g., an HLA polypeptide comprising an amino acid sequence having at least 95%, at least 98%, at least 99%, or 100%, amino acid sequence identity to the amino acid sequence depicted in FIG. 4A. In some cases, the HLA heavy chain polypeptide is an HLA-A*1101 polypeptide comprising Y84C and A236C substitutions (e.g., as depicted in FIG. 4C). In some cases, the HLA heavy chain polypeptide is an HLA-A*2402 polypeptide; e.g., an HLA polypeptide comprising an amino acid sequence having at least 95%, at least 98%, at least 99%, or 100%, amino acid sequence identity to the amino acid sequence depicted in FIG. 5A. In some cases, the HLA heavy chain polypeptide is an HLA-A*2402 polypeptide comprising Y84C and A236C substitutions (e.g., as depicted in FIG. 5C). In some cases, the HLA heavy chain polypeptide is an HLA-A*3303 polypeptide; e.g., an HLA polypeptide comprising an amino acid sequence having at least 95%, at least 98%, at least 99%, or 100%, amino acid sequence identity to the amino acid sequence depicted in FIG. 6A. In some cases, the HLA heavy chain polypeptide is an HLA-A*3303 polypeptide comprising Y84C and A236C substitutions (e.g., as depicted in FIG. 6C). In some cases, the HLA heavy chain polypeptide is an HLA-E polypeptide. In some cases, the HLA-E polypeptide comprises an amino acid sequence having at least 95%, at least 98%, at least 99%, or 100%, amino acid sequence identity to the HLA-E amino acid sequence depicted in any one of FIG. 16A-16D. In some cases, the HLA-E polypeptide comprises Y84C and A236C substitutions (e.g., as depicted in FIG. 16B and FIG. 16D). In some cases, the HLA heavy chain polypeptide is an HLA-E polypeptide, and the peptide epitope is a SARS-CoV2 peptide having the amino acid sequence VMPLSAPTL (SEQ ID NO:914). In some cases, the HLA heavy chain polypeptide is an HLA-E polypeptide, and the peptide epitope is a SARS-CoV2 peptide having the amino acid sequence YLQPRTFLL (SEQ ID NO:218). In some cases, the scFv polypeptide or the nanobody specifically binds CD19. In some cases, the scFv polypeptide or the nanobody specifically binds mesothelin. In some cases, the scFv polypeptide specifically binds TROP-2. In some cases, the scFv polypeptide or the nanobody specifically binds CD28. In some cases, the scFv polypeptide or the nanobody specifically binds Her2. In some cases, the scFv polypeptide or the nanobody specifically binds PSMA. In some cases, the scFv polypeptide or the nanobody specifically binds CD22. In some cases, the scFv polypeptide or the nanobody specifically binds BCMA. In some cases, the scFv polypeptide or the nanobody specifically binds MUC1. In some cases, the scFv polypeptide or the nanobody specifically binds MUC16. In some cases, the scFv polypeptide or the nanobody specifically binds claudin-18.2. In some cases, the TMMP comprises a scFv. As one non-limiting example, in some cases, a TMMP comprises: a) a first polypeptide comprising the amino acid sequence depicted in FIG. 41B; and b) a second polypeptide comprising the amino acid sequence depicted in FIG. 41A. As another non-limiting example, in some cases, a TMMP comprises: a) a first polypeptide comprising the amino acid sequence depicted in FIG. 41C; and b) a second polypeptide comprising the amino acid sequence depicted in FIG. 41A.

Although embodiments including a SARS-CoV-2 are illustrated herein, as discussed above, peptide epitopes from other pathogens could be employed in TMMPs that also comprise one or more MODs. For example, other suitable epitopes include, but are not limited to, epitopes present in an infectious disease agent, e.g., an epitope presented by a virus-encoded polypeptide or bacteria. Examples of viral infectious disease agents include, e.g., Adenoviruses, Adeno-associated virus, Alphaviruses (Togaviruses), Eastern equine encephalitis virus, Eastern equine encephalomyelitis virus, Venezuelan equine encephalomyelitis vaccine strain TC-83, Western equine encephalomyelitis virus, Arenaviruses, Lymphocytic choriomeningitis virus (non-neurotropic strains), Tacaribe virus complex, Bunyaviruses, Bunyamwera virus, Rift Valley fever virus vaccine strain MP-12, Chikungunya virus, Calciviruses, Coronaviruses, Cowpox virus, Flaviviruses (Togaviruses)—Group B Arboviruses, Dengue virus serotypes 1, 2, 3, and 4, Yellow fever virus vaccine strain 17D, Hepatitis A, B, C, D, and E viruses, the Cytomegalovirus, Epstein Barr virus, Eastern Equine encephalitis virus, Herpes simplex types 1 and 2, Herpes zoster, Human herpesvirus types 6 and 7, hepatitis C virus (HVC), hepatitis B virus (HBV), Influenza viruses types A, B, and C, Papovaviruses, Newcastle disease virus, Measles virus, Mumps virus, Parainfluenza viruses types 1, 2, 3, and 4, polyomaviruses (JC virus, BK virus), Respiratory syncytial virus, Human parvovirus (B 19), Coxsackie viruses types A and B, Echoviruses, Polioviruses, Rhinoviruses, Alastrim (Variola minor virus), Smallpox (Variola major virus), Whitepox Reoviruses, Coltivirus, human Rotavirus, and Orbivirus (Colorado tick fever virus), Rabies virus, Vesicular stomatitis virus, Rubivirus (rubella), Semliki Forest virus, St. Louis encephalitis virus, Venezuelan equine encephalitis virus, Venezuelan equine encephalomyelitis virus, Arenaviruses (a.k.a. South American Hemorrhagic Fever virus), Flexal, Lymphocytic choriomeningitis virus (LCM) (neurotropic strains), Hantaviruses including Hantaan virus, Rift Valley fever virus, Japanese encephalitis virus, Yellow fever virus, Monkeypox virus, Human immunodeficiency virus (HIV) types 1 and 2, Human T cell lymphotropic virus (HTLV) types 1 and 2, Simian immunodeficiency virus (SIV), Vesicular stomatitis virus, Guanarito virus, Lassa fever virus, Junin virus, Machupo virus, Sabia, Crimean-Congo hemorrhagic fever virus, Ebola viruses, Marburg virus, Tick-borne encephalitis virus complex (flavi) including Central European tick-borne encephalitis, Far Eastern tick-borne encephalitis, Hanzalova, Hypr, Kumlinge, Kyasanur Forest disease, Omsk hemorrhagic fever, and Russian Spring Summer encephalitis viruses, Herpesvirus simiae (Herpes B or Monkey B virus), Cercopithecine herpesvirus 1 (Herpes B virus), Equine morbillivirus (Hendra and Hendra-like viruses), Nipah virus, Variola major virus (Smallpox virus), Variola minor virus (Alastrim), African swine fever virus, African horse sickness virus, Akabane virus, Avian influenza virus (highly pathogenic), Blue tongue virus, Camel pox virus, Classical swine fever virus, Cowdria ruminantium (heartwater), Foot and mouth disease virus, Goat pox virus, Japanese encephalitis virus, Lumpy skin disease virus, Malignant catarrhal fever virus, Menangle virus, Newcastle disease virus (VVND), Vesicular stomatitis virus (exotic), and Zika virus. Antigens encoded by such viruses are known in the art; a peptide epitope suitable for use in a TMMP of the present disclosure can include a peptide from any known viral antigen. In some cases, a viral epitope is an epitope present in a viral antigen encoded by a virus other than SARS-CoV-2 that infects a majority of the human population, where such viruses include, e.g., cytomegalovirus (CMV), Epstein-Barr virus (EBV), human papilloma virus, adenovirus, and the like. See, e.g., published PCT application WO2020/243315 (Cue Biopharma, Inc.) at paragraphs [00321]-[00338]. Accordingly, this disclosure expressly contemplates TMMPs comprising an epitope other than a SARS-CoV-2 epitope.

Tmmps without an Immunomodulatory Polypeptide

The present disclosure provides a TMMP comprising: a) a first polypeptide; and b) a second polypeptide, wherein the TMMP comprises: a peptide epitope; a first MHC polypeptide; a second MHC polypeptide; an immunoglobulin (Ig) Fc polypeptide or a non-Ig scaffold; and a TTP, where the TMMP does not include an immunomodulatory polypeptide (e.g., does not comprise a MOD as set forth in Table 2; e.g., does not comprise an IL-2 polypeptide, a 4-1BBL polypeptide, etc.). Such a TMMP may also be referred to herein as a “MODless TMMP.” The components of a MODless TMMP (e.g., peptide epitope, first MHC polypeptide, second MHC polypeptide, Ig Fc polypeptide or non-Ig scaffold, and TTP) can be arranged in a variety of configurations. The components (other than MODs) are as described above.

In some cases, the present disclosure provides a protein comprising at least one heterodimer comprising: a) a first polypeptide comprising: i) a SARS-CoV-2 peptide, wherein the SARS-CoV-2 peptide has a length of from about 4 amino acids to about 25 amino acids, e.g., from 8 to 12 amino acids; and a β2M polypeptide; b) a second polypeptide an MHC class I heavy chain polypeptide; c) an Ig Fc polypeptide or a non-Ig scaffold, where the first or the second polypeptide comprises the Ig Fc polypeptide or the non-Ig scaffold; and d) a TTP, where the first and/or the second polypeptide comprises the TTP, wherein the protein does not include an immunomodulatory polypeptide. One or more of the individual components of the first and/or second polypeptide can be linked together by one or more linkers. Suitable SARS-CoV-2 peptides, MHC class I heavy chain polypeptides, and Ig Fc polypeptides are described above. Such a protein can also be referred to as a “MOD-less TMMP.”

In some cases, a MOD-less TMMP is dimerized. Thus, present disclosure thus provides a protein that is a dimerized MOD-less TMMP comprising two MOD-less TMMPs that are covalently linked to each other. The covalent linkage of the dimer can be one or more disulfide bonds between an Ig Fc polypeptide in the first MOD-less TMMP and an Ig Fc polypeptide in the second MOD-less TMMP.

In some cases, a MODless TMMP comprises: a) a first polypeptide comprising: (i) a peptide epitope, where the peptide epitope is a SARS-CoV-2 peptide; and (ii) a first MHC polypeptide, where the first MHC polypeptide is a β2M polypeptide; and b) a second polypeptide comprising: (i) a TTP; (ii) a second MHC polypeptide, where the second MHC polypeptide is an MHC class I heavy chain polypeptide; and (iii) an Ig Fc polypeptide. A peptide linker may be interposed between one or more of: (i) the peptide epitope and the β2M polypeptide; (ii) the MHC class I heavy chain polypeptide and the TTP; (iii) the MHC class I heavy chain polypeptide and the Ig Fc polypeptide. In some cases, the first polypeptide comprises, in order from N-terminus to C-terminus: (i) a SARS-CoV-2 peptide; and (ii) a β2M polypeptide. In some cases, the first polypeptide comprises, in order from N-terminus to C-terminus: (i) a SARS-CoV-2 peptide; (ii) a peptide linker, where the peptide linker optionally may include a Cys residue; and (iii) a β2M polypeptide. In some cases, the second polypeptide comprises, in order from N-terminus to C-terminus: (i) a TTP; (ii) an optional linker; (iii) an MHC class I heavy chain polypeptide; (iv) an optional linker; and (v) an Ig Fc polypeptide. In some cases, the second polypeptide comprises, in order from N-terminus to C-terminus: (i) an MHC class I heavy chain polypeptide; (ii) an optional linker; (iii) an Ig Fc polypeptide; (iv) an optional linker; and (v) a TTP. In some cases, the second polypeptide comprises, in order from N-terminus to C-terminus: (i) an MHC class I heavy chain polypeptide; (ii) an optional linker; (iii) a TTP; (iv) an optional linker; and (v) an Ig Fc polypeptide. In some cases, the β2M polypeptide comprises a Cys residue, e.g., a Cys residue at position 12 according to the amino acid numbering of the β2M amino acid sequence depicted in FIG. 1B. In some cases, the MHC class I heavy chain polypeptide comprises a Cys residue at position 84 and/or a Cys residue at position 236.

The following are non-limiting examples of MODless TMMPs.

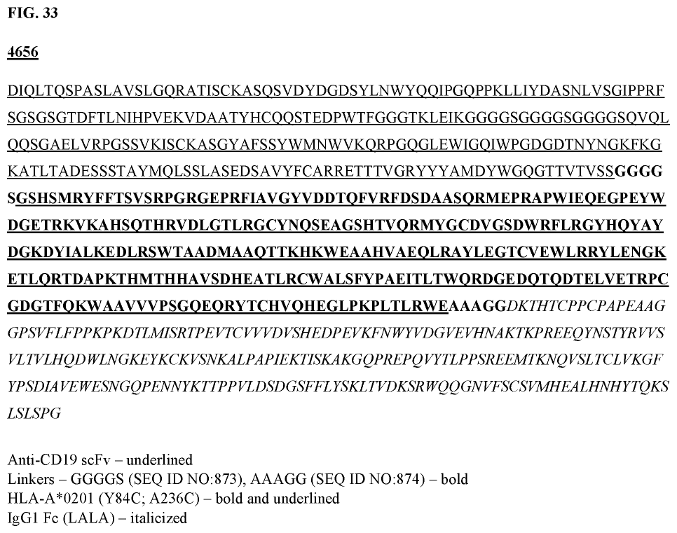

In some cases, a MODless TMMP comprises: a) a first polypeptide comprising: (i) a SARS-CoV-2 peptide; and (ii) a β2M polypeptide comprising an amino acid sequence having at least 90%, at least 95%, at least 98%, at least 99%, or 100%, amino acid sequence identity to the following β2M amino acid sequence: IQRTPKIQVYSCHPAENGKSNFLNCYVSGFHPSDIEVDLLKNGERIEKVEHSDLSFSKDWSFYLL YYTEFTPTEKDEYACRVNHVTLSQPKIVKWDRDM (SEQ ID NO:2), where amino acid 12 is a Cys; and b) a second polypeptide comprising: (i) an anti-CD19 scFv polypeptide comprising an amino acid sequence having at least 90%, at least 95%, at least 98%, at least 99%, or 100%, amino acid sequence identity to the anti-CD19 scFv polypeptide amino acid sequence depicted in FIG. 32C; (ii) an MHC class I heavy chain polypeptide comprising an amino acid sequence having at least 90%, at least 95%, at least 98%, at least 99%, or 100%, amino acid sequence identity to the HLA-A*0201 (Y84C; A236C) amino acid sequence depicted in FIG. 32D, where amino acid 84 is Cys and where amino acid 236 is Cys; and (iii) an Ig Fc polypeptide comprising an amino acid sequence having at least 90%, at least 95%, at least 98%, at least 99%, or 100%, amino acid sequence identity to the Ig Fc (L14A; L15A) amino acid sequence depicted in FIG. 32E, where amino acid 14 is an Ala and where amino acid 15 is an Ala. In some cases, the first polypeptide comprises a Cys-containing peptide linker between the SARS-CoV-2 peptide and the β2M polypeptide. As an example, in some cases, the first polypeptide comprises, between the SARS-CoV-2 peptide and the β2M polypeptide, a peptide linker of the following amino acid sequence: GCGGSGGGGSGGGGS (SEQ ID NO:882). In some cases, the SARS-CoV-2 peptide has the amino acid sequence YLQPRTFLL (SEQ ID NO:218). In some cases, the first polypeptide comprises an amino acid sequence having at least 90%, at least 95%, at least 98%, at least 99%, or 100%, amino acid sequence identity to the amino acid sequence of the polypeptide designated “4700” and depicted in FIG. 32B. In some cases, the first polypeptide comprises an amino acid sequence having at least 90%, at least 95%, at least 98%, at least 99%, or 100%, amino acid sequence identity to the amino acid sequence of the polypeptide designated “4656” and depicted in FIG. 33.

In some cases, a MODless TMMP comprises: a) a first polypeptide comprising: (i) a SARS-CoV-2 peptide; and (ii) a β2M polypeptide comprising an amino acid sequence having at least 90%, at least 95%, at least 98%, at least 99%, or 100%, amino acid sequence identity to the following β2M amino acid sequence: IQRTPKIQVYSCHPAENGKSNFLNCYVSGFHPSDIEVDLLKNGERIEKVEHSDLSFSKDWSFYLL YYTEFTPTEKDEYACRVNHVTLSQPKIVKWDRDM (SEQ ID NO:2), where amino acid 12 is a Cys; and b) a second polypeptide comprising: (i) an anti-CD19 scFv polypeptide comprising an amino acid sequence having at least 90%, at least 95%, at least 98%, at least 99%, or 100%, amino acid sequence identity to the anti-CD19 scFv polypeptide amino acid sequence depicted in FIG. 32C; (ii) an MHC class I heavy chain polypeptide comprising an amino acid sequence having at least 90%, at least 95%, at least 98%, at least 99%, or 100%, amino acid sequence identity to the HLA-E (Y84C; A236C) amino acid sequence depicted in FIG. 16B or FIG. 16D, where amino acid 84 is Cys and where amino acid 236 is Cys; and (iii) an Ig Fc polypeptide comprising an amino acid sequence having at least 90%, at least 95%, at least 98%, at least 99%, or 100%, amino acid sequence identity to the Ig Fc (L14A; L15A) amino acid sequence depicted in FIG. 32E, where amino acid 14 is an Ala and where amino acid 15 is an Ala. In some cases, the first polypeptide comprises a Cys-containing peptide linker between the SARS-CoV-2 peptide and the β2M polypeptide. As an example, in some cases, the first polypeptide comprises, between the SARS-CoV-2 peptide and the β2M polypeptide, a peptide linker of the following amino acid sequence: GCGGSGGGGSGGGGS (SEQ ID NO:882). In some cases, the SARS-CoV-2 peptide has the amino acid sequence VMPLSAPTL (SEQ ID NO:914).

Although MODless embodiments including a SARS-CoV-2 are illustrated herein, those skilled in the art will readily understand that other peptide epitopes could be employed in MODless TMMPs. For example, other suitable epitopes include, but are not limited to, epitopes present in an infectious disease agent, e.g., an epitope presented by a virus-encoded polypeptide. Examples of viral infectious disease agents include, e.g., Adenoviruses, Adeno-associated virus, Alphaviruses (Togaviruses), Eastern equine encephalitis virus, Eastern equine encephalomyelitis virus, Venezuelan equine encephalomyelitis vaccine strain TC-83, Western equine encephalomyelitis virus, Arenaviruses, Lymphocytic choriomeningitis virus (non-neurotropic strains), Tacaribe virus complex, Bunyaviruses, Bunyamwera virus, Rift Valley fever virus vaccine strain MP-12, Chikungunya virus, Calciviruses, Coronaviruses, Cowpox virus, Flaviviruses (Togaviruses)—Group B Arboviruses, Dengue virus serotypes 1, 2, 3, and 4, Yellow fever virus vaccine strain 17D, Hepatitis A, B, C, D, and E viruses, the Cytomegalovirus, Epstein Barr virus, Eastern Equine encephalitis virus, Herpes simplex types 1 and 2, Herpes zoster, Human herpesvirus types 6 and 7, hepatitis C virus (HVC), hepatitis B virus (HBV), Influenza viruses types A, B, and C, Papovaviruses, Newcastle disease virus, Measles virus, Mumps virus, Parainfluenza viruses types 1, 2, 3, and 4, polyomaviruses (JC virus, BK virus), Respiratory syncytial virus, Human parvovirus (B 19), Coxsackie viruses types A and B, Echoviruses, Polioviruses, Rhinoviruses, Alastrim (Variola minor virus), Smallpox (Variola major virus), Whitepox Reoviruses, Coltivirus, human Rotavirus, and Orbivirus (Colorado tick fever virus), Rabies virus, Vesicular stomatitis virus, Rubivirus (rubella), Semliki Forest virus, St. Louis encephalitis virus, Venezuelan equine encephalitis virus, Venezuelan equine encephalomyelitis virus, Arenaviruses (a.k.a. South American Hemorrhagic Fever virus), Flexal, Lymphocytic choriomeningitis virus (LCM) (neurotropic strains), Hantaviruses including Hantaan virus, Rift Valley fever virus, Japanese encephalitis virus, Yellow fever virus, Monkeypox virus, Human immunodeficiency virus (HIV) types 1 and 2, Human T cell lymphotropic virus (HTLV) types 1 and 2, Simian immunodeficiency virus (SIV), Vesicular stomatitis virus, Guanarito virus, Lassa fever virus, Junin virus, Machupo virus, Sabia, Crimean-Congo hemorrhagic fever virus, Ebola viruses, Marburg virus, Tick-borne encephalitis virus complex (flavi) including Central European tick-borne encephalitis, Far Eastern tick-borne encephalitis, Hanzalova, Hypr, Kumlinge, Kyasanur Forest disease, Omsk hemorrhagic fever, and Russian Spring Summer encephalitis viruses, Herpesvirus simiae (Herpes B or Monkey B virus), Cercopithecine herpesvirus 1 (Herpes B virus), Equine morbillivirus (Hendra and Hendra-like viruses), Nipah virus, Variola major virus (Smallpox virus), Variola minor virus (Alastrim), African swine fever virus, African horse sickness virus, Akabane virus, Avian influenza virus (highly pathogenic), Blue tongue virus, Camel pox virus, Classical swine fever virus, Cowdria ruminantium (heartwater), Foot and mouth disease virus, Goat pox virus, Japanese encephalitis virus, Lumpy skin disease virus, Malignant catarrhal fever virus, Menangle virus, Newcastle disease virus (VVND), Vesicular stomatitis virus (exotic), and Zika virus. Antigens encoded by such viruses are known in the art; a peptide epitope suitable for use in a TMMP of the present disclosure can include a peptide from any known viral antigen. In some cases, a viral epitope is an epitope present in a viral antigen encoded by a virus that infects a majority of the human population, where such viruses include, e.g., cytomegalovirus (CMV), Epstein-Barr virus (EBV), human papilloma virus, adenovirus, and the like. See, e.g., published PCT application WO2020/243315 (Cue Biopharma, Inc.) at paragraphs [00321]-[00338]. Accordingly, this disclosure expressly contemplates MODless TMMPs comprising an epitope other than a SARS-CoV-2 epitope.

For example, in some cases, a MODless TMMP comprises: a) a first polypeptide comprising: (i) a viral peptide epitope, e.g., the peptide epitope is a CMV peptide; and (ii) a first MHC polypeptide, where the first MHC polypeptide is a β2M polypeptide; and b) a second polypeptide comprising: (i) a TTP; (ii) a second MHC polypeptide, where the second MHC polypeptide is an MHC class I heavy chain polypeptide; and (iii) an Ig Fc polypeptide. A peptide linker may be interposed between one or more of: (i) the peptide epitope and the β2M polypeptide; (ii) the MHC class I heavy chain polypeptide and the TTP; (iii) the MHC class I heavy chain polypeptide and the Ig Fc polypeptide. In some cases, the first polypeptide comprises, in order from N-terminus to C-terminus: (i) a CMV peptide; and (ii) a β2M polypeptide. In some cases, the first polypeptide comprises, in order from N-terminus to C-terminus: (i) a viral peptide epitope, e.g., a CMV peptide epitops; (ii) a peptide linker, where the peptide linker may include a Cys residue; and (iii) a β2M polypeptide. In some cases, the second polypeptide comprises, in order from N-terminus to C-terminus: (i) a TTP; (ii) an optional linker; (iii) an MHC class I heavy chain polypeptide; (iv) an optional linker; and (v) an Ig Fc polypeptide. In some cases, the second polypeptide comprises, in order from N-terminus to C-terminus: (i) an MHC class I heavy chain polypeptide; (ii) an optional linker; (iii) an Ig Fc polypeptide; (iv) an optional linker; and (v) a TTP. In some cases, the second polypeptide comprises, in order from N-terminus to C-terminus: (i) an MHC class I heavy chain polypeptide; (ii) an optional linker; (iii) a TTP; (iv) an optional linker; and (v) an Ig Fc polypeptide. In some cases, the β2M polypeptide comprises a Cys residue, e.g., a Cys residue at position 12 according to the amino acid numbering of the β2M amino acid sequence depicted in FIG. 1B. In some cases, the MHC class I heavy chain polypeptide comprises a Cys residue at position 84 and/or a Cys residue at position 236. In some cases, the peptide epitope is a CMV peptide that has the amino acid sequence NLVPMVATV (SEQ ID NO:913).

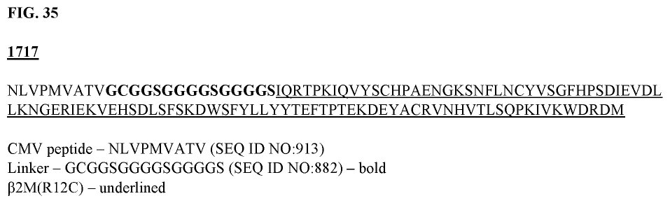

In some cases, a MODless TMMP comprises: a) a first polypeptide comprising: (i) a CMV peptide; and (ii) a β2M polypeptide comprising an amino acid sequence having at least 90%, at least 95%, at least 98%, at least 99%, or 100%, amino acid sequence identity to the following β2M amino acid sequence: IQRTPKIQVYSCHPAENGKSNFLNCYVSGFHPSDIEVDLLKNGERIEKVEHSDLSFSKDWSFYLL YYTEFTPTEKDEYACRVNHVTLSQPKIVKWDRDM (SEQ ID NO:2), where amino acid 12 is a Cys;
and b) a second polypeptide comprising: (i) an anti-CD19 scFv polypeptide comprising an amino acid sequence having at least 90%, at least 95%, at least 98%, at least 99%, or 100%, amino acid sequence identity to the anti-CD19 scFv polypeptide amino acid sequence depicted in FIG. 32C; (ii) an MHC class I heavy chain polypeptide comprising an amino acid sequence having at least 90%, at least 95%, at least 98%, at least 99%, or 100%, amino acid sequence identity to the HLA-A*0201 (Y84C; A236C) amino acid sequence depicted in FIG. 32D, where amino acid 84 is Cys and where amino acid 236 is Cys; and (iii) an Ig Fc polypeptide comprising an amino acid sequence having at least 90%, at least 95%, at least 98%, at least 99%, or 100%, amino acid sequence identity to the Ig Fc (L14A; L15A) amino acid sequence depicted in FIG. 32E, where amino acid 14 is an Ala and where amino acid 15 is an Ala. In some cases, the CMV peptide has the amino acid sequence NLVPMVATV (SEQ ID NO:913). In some cases, the first polypeptide comprises a Cys-containing peptide linker between the CMV peptide and the β2M polypeptide. As an example, in some cases, the first polypeptide comprises, between the CMV peptide and the β2M polypeptide, a peptide linker of the following amino acid sequence: GCGGSGGGGSGGGGS (SEQ ID NO:882). In some cases, the first polypeptide comprises an amino acid sequence having at least 90%, at least 95%, at least 98%, at least 99%, or 100%, amino acid sequence identity to the amino acid sequence of the polypeptide designated “1717” and depicted in FIG. 35. In some cases, the first polypeptide comprises an amino acid sequence having at least 90%, at least 95%, at least 98%, at least 99%, or 100%, amino acid sequence identity to the amino acid sequence of the polypeptide designated “4656” and depicted in FIG. 33.

### Nucleic Acids

The present disclosure provides a nucleic acid comprising a nucleotide sequence encoding a TMMP. The present disclosure provides a nucleic acid comprising a nucleotide sequence encoding a TMMP.

The present disclosure provides nucleic acids comprising nucleotide sequences encoding a TMMP. In some cases, the individual polypeptide chains of a TMMP are encoded in separate nucleic acids. In some cases, all polypeptide chains of a TMMP are encoded in a single nucleic acid. In some cases, a first nucleic acid comprises a nucleotide sequence encoding a first polypeptide of a TMMP; and a second nucleic acid comprises a nucleotide sequence encoding a second polypeptide of a TMMP. In some cases, single nucleic acid comprises a nucleotide sequence encoding a first polypeptide of a TMMP and a second polypeptide of a TMMP.

### Separate Nucleic Acids Encoding Individual Polypeptide Chains of a Multimeric Polypeptide

The present disclosure provides nucleic acids comprising nucleotide sequences encoding a TMMP. As noted above, in some cases, the individual polypeptide chains of a TMMP are encoded in separate nucleic acids. In some cases, nucleotide sequences encoding the separate polypeptide chains of a TMMP are operably linked to transcriptional control elements, e.g., promoters, such as promoters that are functional in a eukaryotic cell, where the promoter can be a constitutive promoter or an inducible promoter.

For example, the present disclosure provides a first nucleic acid and a second nucleic acid, where the first nucleic acid comprises a nucleotide sequence encoding a first polypeptide of a TMMP, where the first polypeptide comprises, in order from N-terminus to C-terminus: a) a peptide epitope; b) a first MHC polypeptide; and c) an immunomodulatory polypeptide (e.g., a reduced-affinity variant, as described above); and where the second nucleic acid comprises a nucleotide sequence encoding a second polypeptide of a TMMP, where the second polypeptide comprises, in order from N-terminus to C-terminus: a) a second MHC polypeptide; b) an Ig Fc polypeptide; and c) a TTP. Suitable peptide epitopes, MHC polypeptides, immunomodulatory polypeptides, Ig Fc polypeptides, and TTPs are described above. In some cases, the nucleotide sequences encoding the first and the second polypeptides are operably linked to transcriptional control elements. In some cases, the transcriptional control element is a promoter that is functional in a eukaryotic cell. In some cases, the nucleic acids are present in separate expression vectors.

The present disclosure provides a first nucleic acid and a second nucleic acid, where the first nucleic acid comprises a nucleotide sequence encoding a first polypeptide of a TMMP, where the first polypeptide comprises, in order from N-terminus to C-terminus: a) a peptide epitope; and b) a first MHC polypeptide; and where the second nucleic acid comprises a nucleotide sequence encoding a second polypeptide of a TMMP, where the second polypeptide comprises, in order from N-terminus to C-terminus: a) an immunomodulatory polypeptide (e.g., a reduced-affinity variant as described above); b) a second MHC polypeptide; c) an Ig Fc polypeptide; and d) a TTP. Suitable peptide epitopes, MHC polypeptides, immunomodulatory polypeptides, and Ig Fc polypeptides, are described above. In some cases, the nucleotide sequences encoding the first and the second polypeptides are operably linked to transcriptional control elements. In some cases, the transcriptional control element is a promoter that is functional in a eukaryotic cell. In some cases, the nucleic acids are present in separate expression vectors.

### Nucleic Acid Encoding Two or More Polypeptides Present in a Multimeric Polypeptide

The present disclosure provides a nucleic acid comprising nucleotide sequences encoding at least the first polypeptide and the second polypeptide of a TMMP. In some cases, where a TMMP includes a first, second, and third polypeptide, the nucleic acid includes a nucleotide sequence encoding the first, second, and third polypeptides. In some cases, the nucleotide sequences encoding the first polypeptide and the second polypeptide of a TMMP includes a proteolytically cleavable linker interposed between the nucleotide sequence encoding the first polypeptide and the nucleotide sequence encoding the second polypeptide. In some cases, the nucleotide sequences encoding the first polypeptide and the second polypeptide of a TMMP includes an internal ribosome entry site (IRES) interposed between the nucleotide sequence encoding the first polypeptide and the nucleotide sequence encoding the second polypeptide. In some cases, the nucleotide sequences encoding the first polypeptide and the second polypeptide of a TMMP includes a ribosome skipping signal (or cis-acting hydrolase element, CHYSEL) interposed between the nucleotide sequence encoding the first polypeptide and the nucleotide sequence encoding the second polypeptide. Examples of nucleic acids are described below, where a proteolytically cleavable linker is provided between nucleotide sequences encoding the first polypeptide and the second polypeptide of a TMMP; in any of these embodiments, an IRES or a ribosome skipping signal can be used in place of the nucleotide sequence encoding the proteolytically cleavable linker.

In some cases, a first nucleic acid (e.g., a recombinant expression vector, an mRNA, a viral RNA, etc.) comprises a nucleotide sequence encoding a first polypeptide chain of a TMMP; and a second nucleic acid (e.g., a recombinant expression vector, an mRNA, a viral RNA, etc.) comprises a nucleotide sequence encoding a second polypeptide chain of a TMMP. In some cases, the nucleotide sequence encoding the first polypeptide, and the second nucleotide sequence encoding the second polypeptide, are each operably linked to transcriptional control elements, e.g., promoters, such as promoters that are functional in a eukaryotic cell, where the promoter can be a constitutive promoter or an inducible promoter.

The present disclosure provides a nucleic acid comprising a nucleotide sequence encoding a recombinant polypeptide, where the recombinant polypeptide comprises, in order from N-terminus to C-terminus: a) a peptide epitope; b) a first MHC polypeptide; c) an immunomodulatory polypeptide (e.g., a reduced-affinity variant as described above); d) a proteolytically cleavable linker; e) a second MHC polypeptide; f) an Ig Fc polypeptide; and g) a TTP. The present disclosure provides a nucleic acid comprising a nucleotide sequence encoding a recombinant polypeptide, where the recombinant polypeptide comprises, in order from N-terminus to C-terminus: a) a first leader peptide; b) the epitope; c) the first MHC polypeptide; d) the immunomodulatory polypeptide (e.g., a reduced-affinity variant as described above); e) the proteolytically cleavable linker; f) a second leader peptide; g) the second MHC polypeptide; h) the Ig Fc polypeptide; and i) the TTP. The present disclosure provides a nucleic acid comprising a nucleotide sequence encoding a recombinant polypeptide, where the recombinant polypeptide comprises, in order from N-terminus to C-terminus: a) an epitope; b) a first MHC polypeptide; c) a proteolytically cleavable linker; d) an immunomodulatory polypeptide (e.g., a reduced-affinity variant as described above); e) a second MHC polypeptide; f) an Ig Fc polypeptide; and g) a TTP.

The present disclosure provides a nucleic acid comprising a nucleotide sequence encoding a recombinant polypeptide, where the recombinant polypeptide comprises, in order from N-terminus to C-terminus: a) an epitope; b) a first MHC polypeptide; c) a proteolytically cleavable linker; d) an immunomodulatory polypeptide (e.g., a reduced-affinity variant as described above); e) a TTP; f) a second MHC polypeptide; and g) an Ig Fc polypeptide.

In some cases, the first leader peptide and the second leader peptide are a 32-M leader peptide. In some cases, the nucleotide sequence is operably linked to a transcriptional control element. In some cases, the transcriptional control element is a promoter that is functional in a eukaryotic cell.

Suitable MHC polypeptides are described above. In some cases, the first MHC polypeptide is a β2-microglobulin polypeptide; and wherein the second MHC polypeptide is an MHC class I heavy chain polypeptide. In some cases, the β2-microglobulin polypeptide comprises an amino acid sequence having at least 85% amino acid sequence identity to a β2M amino acid sequence depicted in FIG. 4. In some cases, the MHC class I heavy chain polypeptide is an HLA-A, HLA-B, HLA-C, HLA-E, HLA-F, HLA-G, HLA-K, or HLA-L heavy chain.

Suitable Fc polypeptides are described above. In some cases, the Ig Fc polypeptide is an IgG1 Fc polypeptide, an IgG2 Fc polypeptide, an IgG3 Fc polypeptide, an IgG4 Fc polypeptide, an IgA Fc polypeptide, or an IgM Fc polypeptide. In some cases, the Ig Fc polypeptide comprises an amino acid sequence having at least 85% amino acid sequence identity to an amino acid sequence depicted in FIGS. 3A-3G.

Suitable immunomodulatory polypeptides are described above.

Suitable proteolytically cleavable linkers are described above. In some cases, the proteolytically cleavable linker comprises an amino acid sequence selected from: a) LEVLFQGP (SEQ ID NO:1204); b) ENLYTQS (SEQ ID NO:1205); c) DDDDK (SEQ ID NO:1206); d) LVPR (SEQ ID NO:1207); and e) GSGATNFSLLKQAGDVEENPGP (SEQ ID NO:1208).

In some cases, a linker between the epitope and the first MHC polypeptide comprises a first Cys residue, and the second MHC polypeptide comprises an amino acid substitution to provide a second Cys residue, such that the first and the second Cys residues provide for a disulfide linkage between the linker and the second MHC polypeptide. In some cases, first MHC polypeptide comprises an amino acid substitution to provide a first Cys residue, and the second MHC polypeptide comprises an amino acid substitution to provide a second Cys residue, such that the first Cys residue and the second Cys residue provide for a disulfide linkage between the first MHC polypeptide and the second MHC polypeptide.

### Recombinant Expression Vectors

The present disclosure provides recombinant expression vectors comprising nucleic acids.

Numerous suitable expression vectors are known to those of skill in the art, and many are commercially available. See, e.g., published PCT Application WO 2020/243315 (Cue Biopharma, Inc.).

### Genetically Modified Host Cells

The present disclosure provides a genetically modified host cell, where the host cell is genetically modified with a nucleic acid.

Suitable host cells include eukaryotic cells, such as yeast cells, insect cells, and mammalian cells. In some cases, the host cell is a cell of a mammalian cell line. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RAT1 cells, mouse L cells (ATCC No. CCLI.3), human embryonic kidney (HEK) cells (ATCC No. CRL1573), HLHepG2 cells, and the like.

In some cases, the host cell is a mammalian cell that has been genetically modified such that it does not synthesize endogenous MHC β2-M.

In some cases, the host cell is a mammalian cell that has been genetically modified such that it does not synthesize endogenous MHC Class I heavy chain. In some cases, the host cell is a mammalian cell that has been genetically modified such that it does not synthesize endogenous MHC β2-M and such that it does not synthesize endogenous MHC Class I heavy chain.

### Methods of Producing a Tmmp

The present disclosure provides methods of producing a TMMP. The methods generally involve culturing, in a culture medium, a host cell that is genetically modified with one or more nucleic acids (e.g., one or more recombinant expression vectors) comprising nucleotide sequences encoding the TMMP; and isolating the TMMP from the genetically modified host cell and/or the culture medium. A host cell that is genetically modified with one or more nucleic acids (e.g., one or more recombinant expression vectors) comprising nucleotide sequences encoding the TMMP is also referred to as an “expression host.” As noted above, in some cases, the individual polypeptide chains of a TMMP are encoded in separate nucleic acids (e.g., separate recombinant expression vectors). In some cases, all polypeptide chains of a TMMP are encoded in a single nucleic acid (e.g., a single recombinant expression vector).

Isolation of the TMMP from the expression host cell (e.g., from a lysate of the expression host cell) and/or the culture medium in which the host cell is cultured, can be carried out using standard methods of protein purification.

For example, a lysate may be prepared of the expression host and the lysate purified using high performance liquid chromatography (HPLC), exclusion chromatography, gel electrophoresis, affinity chromatography, or other purification technique. Alternatively, where the TMMP is secreted from the expression host cell into the culture medium, the TMMP can be purified from the culture medium using HPLC, exclusion chromatography, gel electrophoresis, affinity chromatography, or other purification technique. In some cases, the compositions which are used will comprise at least 80% by weight of the desired product (TMMP), at least about 85% by weight, at least about 95% by weight, or at least about 99.5% by weight, in relation to contaminants related to the method of preparation of the product and its purification. The percentages can be based upon total protein.

In some cases, e.g., where the TMMP comprises an affinity tag, the TMMP can be purified using an immobilized binding partner of the affinity tag.

### Compositions

The present disclosure provides compositions, including pharmaceutical compositions, comprising a TMMP of the present disclosure. The present disclosure provides compositions, including pharmaceutical compositions, comprising a TMMP.

A wide variety of pharmaceutically acceptable ingredients are known in the art and hence are not discussed in detail herein. Moreover, pharmaceutically acceptable ingredients and compositions have been amply described in a variety of publications, including, but not limited to, A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7^{th} ed., Lippincott, Williams, & Wilkins; and Handbook of Pharmaceutical Excipients (2000) A. H. Kibbe et al., eds., 3^{rd} ed. Amer. Pharmaceutical Assoc. Many other publications describing preparation of biopharmaceutical compositions may be consulted.

The composition may be formulated according to the various routes of administration described below. Generally speaking, TMMPs of this disclosure, e.g., a homodimer comprising two TMMPs, will be an aqueous liquid and typically will be administered via an intravenous infusion. In some cases, the pharmaceutical composition comprising the TMMP can be admixed with saline (e.g., 0.9% NaCl) prior to IV administration. Thus, the present disclosure provides a sterile composition comprising: a) a TMMP of the present disclosure; and b) saline (e.g., 0.9% NaCl). Alternatively, it may be administered neat via an intravenous infusion, i.e., without further dilution. Alternatively, the pharmaceutical composition may be formulated so as to be administered by injection.

Conventional and pharmaceutically acceptable routes of administration include intratumoral, peritumoral, intramuscular, intralymphatic, intratracheal, intracranial, subcutaneous, intradermal, topical application, intravenous, intraarterial, rectal, nasal, oral, and other enteral and parenteral routes of administration. As noted above, a pharmaceutical composition comprising a TMMP typically will be administered intravenously, but may also be administered by other routes that involve injection.

### Compositions Comprising a Nucleic Acid or a Recombinant Expression Vector

The present disclosure provides compositions, e.g., pharmaceutical compositions, comprising a nucleic acid or a recombinant expression vector. A wide variety of pharmaceutically acceptable excipients is known in the art and need not be discussed in detail herein. Pharmaceutically acceptable excipients have been amply described in a variety of publications, including, for example, A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

### Methods of Modulating T Cell Activity

The present disclosure provides a method of selectively modulating the activity of an epitope-specific T cell (e.g., a T cell comprising a TCR specific for SARS-CoV-2 (e.g., a SARS-CoV-2 peptide in a complex with MHC polypeptides); and/or a T-cell specific for a cancer-associated antigen), the method comprising contacting the T cell with a TMMP, where contacting the T cell with a TMMP selectively modulates the activity of the epitope-specific T cell. In some cases, the contacting occurs in vitro. In some cases, the contacting occurs in vivo. In some cases, the contacting occurs ex vivo.

Where a TMMP includes an immunomodulatory polypeptide that is an activating polypeptide, contacting the T cell with the TMMP activates the epitope-specific T cell.

In some instances, a TMMP includes: i) an immunomodulatory polypeptide that is an activating polypeptide; ii) a SARS-CoV-2 peptide; and iii) a TTP that targets a cancer cell; and the TMMP is contacted with a T cell that is specific for the SARS-CoV-2 peptide present in the TMMP. In these instances, contacting the SARS-CoV-2-specific T cell with the TMMP activates the SARS-CoV-2-specific T cell and/or increases proliferation of the SARS-CoV-2-specific T cell. In some cases, contacting the SARS-CoV-2-specific T cell with the TMMP increases the number and/or cytotoxic activity of the T cell toward a cancer cell that is targeted by the TTP present in the TMMP. As one non-limiting example, where a TMMP comprises: i) an immunomodulatory polypeptide that is an activating polypeptide (e.g., an IL-2 polypeptide); ii) a SARS-CoV-2 peptide as the peptide epitope; and iii) a TTP that is a scFv that binds Her2, contacting the TMMP with a cytotoxic T cell that binds the SARS-CoV-2 peptide activates the T cell and increases its cytotoxic activity toward a Her2-expressing cancer cell.

The present disclosure provides a method of modulating an immune response in an individual, the method comprising administering to the individual an effective amount of a TMMP. Administering the TMMP induces an epitope-specific T cell response (e.g., a SARS-CoV-2-specific response) and an epitope-non-specific T cell response, where the ratio of the epitope-specific T cell response to the epitope-non-specific T cell response is at least 2:1 or greater, e.g. at least 5:1, 10:1, at least 25:1 or greater. In some cases, the modulating increases a cytotoxic T-cell response to a cancer cell, e.g., a cancer cell expressing an antigen that is targeted by the TTP present in the TMMP.

“Modulating the activity” of a T cell can include, for example, one or more of: i) activating a cytotoxic (e.g., CD8⁺) T cell; ii) inducing cytotoxic activity of a cytotoxic (e.g., CD8⁺) T cell; and/or iii) inducing production and release of a cytotoxin (e.g., a perforin; a granzyme; a granulysin) by a cytotoxic (e.g., CD8⁺) T cell.

The present disclosure provides a method of delivering a costimulatory (i.e., immunomodulatory) polypeptide selectively to target T cell, the method comprising contacting a mixed population of T cells with a TMMP, where the mixed population of T cells comprises the target T cell and non-target T cells, where the target T cell is specific for the epitope present within the TMMP (e.g., where the target T cell is specific for the epitope present within the TMMP), and where the contacting step delivers the one or more costimulatory polypeptides (immunomodulatory polypeptides) present within the TMMP to the target T cell. In some cases, the population of T cells is in vitro. In some cases, the population of T cells is in vivo in an individual. In some cases, the method comprises administering the TMMP to the individual. In some case, the T cell is a cytotoxic T cell. In some cases, the mixed population of T cells is an in vitro population of mixed T cells obtained from an individual, and the contacting step results in activation and/or proliferation of the target T cell, generating a population of activated and/or proliferated target T cells; in some of these instances, the method further comprises administering the population of activated and/or proliferated target T cells to the individual.

The present disclosure provides a method of detecting, in a mixed population of T cells obtained from an individual, the presence of a target T cell that binds an epitope of interest (e.g., a SARS-CoV-2 epitope), the method comprising: a) contacting in vitro the mixed population of T cells with a TMMP, wherein the TMMP comprises the epitope of interest (e.g., the SARS-CoV-2 epitope); and b) detecting activation and/or proliferation of T cells in response to said contacting, wherein activated and/or proliferated T cells indicates the presence of the target T cell.

### Treatment Methods

The present disclosure provides a method of treatment of an individual, the method comprising administering to the individual an amount of a TMMP (e.g., a homodimer TMMP), or one or more nucleic acids encoding the TMMP, effective to treat the individual. Also provided is a TMMP for use in a method of treatment of the human or animal body. In some cases, a treatment method comprises administering to an individual in need thereof one or more recombinant expression vectors comprising nucleotide sequences encoding a TMMP. In some cases, a treatment method comprises administering to an individual in need thereof one or more mRNA molecules comprising nucleotide sequences encoding a TMMP. In some cases, a treatment method comprises administering to an individual in need thereof a TMMP. Conditions that can be treated include, e.g., cancer and autoimmune disorders, as described below.

A TMMP can both: 1) modulate the activity of an epitope-specific T cell (e.g., a T cell specific for the epitope present in the TMMP); and 2) target the TMMP to a target cell. For example, the TTP can cause the TMMP to bind to a cancer cell, where the TMMP engages with and activates a T cell specific for the epitope, e.g., the cytotoxic activity of the T cell is increased, which in turn leads to killing of the cancer cell. For example, in some cases, a TMMP:1) targets the TMMP to a cancer cell, and 2) induces a cytotoxic T cell response to a viral epitope (e.g., SARS-CoV-2 peptide) present in the TMMP.

As noted above, depending on the affinity for a wild-type MOD for its co-MOD, the combination of the reduced affinity of the MOD for its co-MOD, and the affinity of the epitope for a TCR, provides for enhanced selectivity of a TMMP. Thus, for example, a TMMP binds with higher avidity to a first T cell that displays both: i) a TCR specific for the epitope present in the TMMP; and ii) a co-immunomodulatory polypeptide that binds to the immunomodulatory polypeptide present in the TMMP, compared to the avidity to which it binds to a second T cell that displays: i) a TCR specific for an epitope other than the epitope present in the TMMP; and ii) a co-immunomodulatory polypeptide that binds to the immunomodulatory polypeptide present in the TMMP. Variant MODs may be advantageous in multiple circumstance, e.g., where a wild-type MOD has a relatively high affinity for a co-MOD (e.g., IL-2 for IL-2R) and/or can have multiple effects upon binding (e.g., IL-2, which can both activate epitope-specific T cells and upregulate the production of Tregs), or where a MOD can bind to multiple co-MODs (e.g., CD80, which can bind both CD28 and CTLA4). In such cases, reducing the affinity for a co-MOD may be advantageous by decreasing off-target binding of the TMMP, increasing the desired activation of the TMMP and/or decreasing or substantially eliminating the undesired activation of the TMMP.

The present disclosure provides a method of selectively modulating the activity of an epitope-specific T cell in an individual, the method comprising administering to the individual an effective amount of a TMMP, or one or more nucleic acids (e.g., expression vectors; mRNA; etc.) comprising nucleotide sequences encoding the TMMP, where the TMMP selectively modulates the activity of the epitope-specific T cell in the individual. Selectively modulating the activity of an epitope-specific T cell can treat a disease or disorder in the individual. Thus, the present disclosure provides a treatment method comprising administering to an individual in need thereof an effective amount of a TMMP.

Cancers that can be treated with a method include any cancer that can be targeted with a TTP. Cancers that can be treated with a method include carcinomas, sarcomas, melanoma, leukemias, lymphomas and multiple myeloma. Cancers that can be treated with a method include solid tumors, and cancers that begin in blood-forming tissue, i.e., hematological cancers such as leukemias, lymphomas and multiple myeloma. Cancers that can be treated with a method include metastatic cancers.

Cancers that that can be treated with a method include cancers that have escaped/evaded the immune system through HLA loss. Such cancers, which lack cell-surface expression of the class I major histocompatibility complex, thus can escape recognition by cancer-specific cytotoxic T cells. Because the anti-cancer effect of the TMMPs described herein does not depend on recognition of the cancer cells by cancer-specific cytotoxic T cells, but rather on the recognition by T cells that have a TCR that is specific for the pMHC on the TMMP, HLA loss does not hinder the TMMPs from killing cancer cells.

Carcinomas that can treated by a method disclosed herein include, but are not limited to, esophageal carcinoma, hepatocellular carcinoma, basal cell carcinoma (a form of skin cancer), squamous cell carcinoma (various tissues), bladder carcinoma, including transitional cell carcinoma (a malignant neoplasm of the bladder), bronchogenic carcinoma, colon carcinoma, colorectal carcinoma, gastric carcinoma, lung carcinoma, including small cell carcinoma and non-small cell carcinoma of the lung, adrenocortical carcinoma, thyroid carcinoma, pancreatic carcinoma, breast carcinoma, ovarian carcinoma, prostate carcinoma, adenocarcinoma, sweat gland carcinoma, sebaceous gland carcinoma, papillary carcinoma, papillary adenocarcinoma, cystadenocarcinoma, medullary carcinoma, renal cell carcinoma, ductal carcinoma in situ or bile duct carcinoma, choriocarcinoma, seminoma, embryonal carcinoma, Wilm's tumor, cervical carcinoma, uterine carcinoma, testicular carcinoma, osteogenic carcinoma, epithelial carcinoma, and nasopharyngeal carcinoma.

Sarcomas that can be treated by a method disclosed herein include, but are not limited to, fibrosarcoma, myxosarcoma, liposarcoma, chondrosarcoma, chordoma, osteogenic sarcoma, osteosarcoma, angiosarcoma, endotheliosarcoma, lymphangiosarcoma, lymphangioendotheliosarcoma, synovioma, mesothelioma, Ewing's sarcoma, leiomyosarcoma, rhabdomyosarcoma, and other soft tissue sarcomas.

Other solid tumors that can be treated by a method disclosed herein include, but are not limited to, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma.

Leukemias that can be amenable to therapy by a method disclosed herein include, but are not limited to, a) chronic myeloproliferative syndromes (neoplastic disorders of multipotential hematopoietic stem cells); b) acute myelogenous leukemias (neoplastic transformation of a multipotential hematopoietic stem cell or a hematopoietic cell of restricted lineage potential; c) chronic lymphocytic leukemias (CLL; clonal proliferation of immunologically immature and functionally incompetent small lymphocytes), including B-cell CLL, T-cell CLL prolymphocytic leukemia, and hairy cell leukemia; and d) acute lymphoblastic leukemias (characterized by accumulation of lymphoblasts). Lymphomas that can be treated using a subject method include, but are not limited to, B-cell lymphomas (e.g., Burkitt's lymphoma); Hodgkin's lymphoma; non-Hodgkin's lymphoma, and the like.

Other cancers that can be treated according to the methods disclosed herein include atypical meningioma, islet cell carcinoma, medullary carcinoma of the thyroid, mesenchymoma, hepatocellular carcinoma, hepatoblastoma, clear cell carcinoma of the kidney, and neurofibroma mediastinum.

Where a TMMP comprises: i) a peptide epitope that when in an MHC/peptide complex of a TMMP presents a viral epitope; and ii) a TTP that targets a cancer-associated antigen, the TMMP can be administered to an individual in need thereof to treat a cancer in the individual, where: i) the TMMP activates a T-cell that is specific for the viral epitope (e.g., a SARS-CoV-2 epitope); and ii) the cancer expresses the cancer epitope bound by the TTP. The present disclosure provides a method of treating cancer in an individual, the method comprising administering to the individual an effective amount of a TMMP, where the TMMP comprises: i) a peptide epitope that when in an MHC/peptide complex of a TMMP presents a viral epitope (e.g., a SARS-CoV-2 epitope); ii) a TTP that targets a cancer-associated antigen; and iii) a stimulatory immunomodulatory polypeptide (e.g., an IL-2 polypeptide; a 4-1BBL polypeptide; etc.).

In some cases, an “effective amount” of a TMMP is an amount that, when administered in one or more doses to an individual in need thereof, reduces the number of cancer cells in the individual. For example, in some cases, an “effective amount” of a TMMP is an amount that, when administered in one or more doses to an individual in need thereof, reduces the number of cancer cells in the individual by at least 10%, at least 15%, at least 20%, at least 25%, at least 30%, at least 40%, at least 50%, at least 60%, at least 70%, at least 80%, at least 90%, or at least 95%, compared to the number of cancer cells in the individual before administration of the TMMP, or in the absence of administration with the TMMP. In some cases, an “effective amount” of a TMMP is an amount that, when administered in one or more doses to an individual in need thereof, reduces the number of cancer cells in the individual to undetectable levels.

In some cases, an “effective amount” of a TMMP is an amount that, when administered in one or more doses to an individual in need thereof, reduces the tumor mass/tumor volume in the individual. In some cases, an “effective amount” of a TMMP is an amount that, when administered in one or more doses to an individual in need thereof, increases survival time of the individual. For example, in some cases, an “effective amount” of a TMMP is an amount that, when administered in one or more doses to an individual in need thereof, increases survival time of the individual by at least 1 month, at least 2 months, at least 3 months, from 3 months to 6 months, from 6 months to 1 year, from 1 year to 2 years, from 2 years to 5 years, from 5 years to 10 years, or more than 10 years, compared to the expected survival time of the individual in the absence of administration with the TMMP.

In some cases, an “effective amount” of a TMMP is an amount that, when administered in one or more doses to an individual in need thereof, either as a monotherapy or as part of a combination therapy, reduces the overall tumor burden in the individual, i.e., the amount of cancer in the body, or alternatively, causes the total tumor burden in the patient to remain relatively stable for a sufficient period of time for the patient to have a confirmed “stable disease” as determined by standard RECIST criteria. See, e.g., Aykan and Ozatli (2020) World J. Clin. Oncol. 11:53.

In some cases, an effective amount of a TMMP is an amount that, when administered in one or more doses to an individual in need thereof, either as a monotherapy or as part of a combination therapy, causes the tumor size to be reduced by a sufficient amount, and for a sufficient period of time, for the patient to have a confirmed “partial response” as determined by standard RECIST criteria.

In some cases, an effective amount of a TMMP is an amount that, when administered in one or more doses to an individual in need thereof (e.g., an individual having a tumor), either as a monotherapy or as part of a combination therapy, causes the tumor size to be reduced by a sufficient amount, and for a sufficient period of time, for the patient to have a confirmed “complete response” as determined by standard RECIST criteria.

As noted above, in some cases, in carrying out a subject treatment method, a TMMP is administered to an individual in need thereof, as the TMMP per se. In other instances, in carrying out a subject treatment method, one or more nucleic acids comprising nucleotide sequences encoding a TMMP is/are administering to an individual in need thereof. Thus, in other instances, one or more nucleic acids, e.g., one or more recombinant expression vectors, is/are administered to an individual in need thereof.

In some cases, a SARS-CoV-2 vaccine (e.g., the Moderna, Pfizer or J&J vaccine) is administered to the patient several days (e.g., 3-14 days or 7-10 days) prior to administering the TMMP in order to increase the percentage of CD8⁺ T cells in the patient that recognize the SARS CoV-2 peptide in the TMMP. Alternatively, prior to administering the TMMP, one or more doses of a TMMP that does not comprise a TTP can be administered to the patient, in order to prime and activate and/or induce proliferation of (and thereby increase the number of) CD8⁺ T cells that recognize the desired SARS CoV-2 peptide in the TMMP comprising the TTP. Alternatively (or additionally), prior to administering the TMMP, one or more doses of a T cell modulatory polypeptide can be administered to the patient, in order to prime and activate and/or induce proliferation of (and thereby increase the number of) CD8⁺ T cells that recognize the desired SARS CoV-2 peptide in the TMMP comprising the TTP. See, e.g., the T cell modulatory polypeptides described in WO2021/195108, published Sep. 30, 2021 (Cue Biopharma, Inc.), WO 2021/195411, published Sep. 30, 2021 (Cue Biopharma, Inc.), and WO 2022/015880, published Jan. 20, 2022 (Cue Biopharma, Inc.).

### Dosages

A suitable dosage can be determined by an attending physician or other qualified medical personnel, based on various clinical factors. As is well known in the medical arts, dosages for any one patient depend upon many factors, including the patient's size, body surface area, age, the particular polypeptide or nucleic acid to be administered, sex of the patient, time, and route of administration, general health, and other drugs being administered concurrently. A TMMP of the present disclosure may be administered in amounts between 1 ng/kg body weight and 20 mg/kg body weight per dose, or higher, e.g. from 0.1 mg/kg body weight to 10 mg/kg body weight, e.g. from 1 mg/kg body weight to 5 mg/kg or from 5 mg/kg body weight to 10 mg/kg body weight; from 10-15 mg/kg, or higher, however, doses below or above this exemplary range are envisioned, especially considering the aforementioned factors. If the regimen is a continuous infusion, it can also be in the range of 1 μg to 10 mg per kilogram of body weight per minute. Generally speaking, a TMMP of the present disclosure can be administered in an amount of from about 1 mg/kg body weight to 20 mg/kg body weight, e.g., from about 1 mg/kg body weight to about 5 mg/kg body weight, from about 5 mg/kg body weight to about 10 mg/kg body weight, from about 10 mg/kg body weight to about 15 mg/kg body weight, or from about 15 mg/kg body weight to about 20 mg/kg body weight. Typical ranges may be from 1 mg/kg body weight to 5 mg/kg body weight or from 5 mg/kg body weight to about 10 mg/kg body weight, e.g., 1, 2, 4, 5, 6, 7 or 8 mg/kg body weight.

Following successful treatment, it may be desirable to have the patient undergo maintenance therapy to prevent the recurrence of the disease state, wherein a TMMP is administered in maintenance doses in the above ranges.

Those of skill will readily appreciate that dose levels can vary as a function of the specific TMMP, the severity of the symptoms and the susceptibility of the subject to side effects. Preferred dosages for a given compound are readily determinable by those of skill in the art by a variety of means.

The frequency of administration of a TMMP can vary depending on any of a variety of factors, but generally speaking will be administered once a week, once every two weeks, once every three weeks, once every four weeks, once per month, or less frequently than once per month, e.g., once every five weeks, once every six weeks, once every two months, once every three months, etc., but also can be administered more frequently than once per week, e.g., twice per week (biw), three times per week (tiw), four times per week, five times per week, six times per week, every other day (qod), or daily (qd). In some cases, the TMMP is administered once every three weeks. Administration generally should be stopped upon disease progression or unacceptable toxicity.

The duration of administration of a TMMP can vary, depending on any of a variety of factors, e.g., patient response, etc. For example, a TMMP can be administered over a period of time ranging from one month to about two months, from about two months to about four months, from about four months to about six months, from about six months to about eight months, from about eight months to about 1 year, from about 1 year to about 2 years, or from about 2 years to about 4 years, or more. Typically, the TMMP will continue to be dosed for at least as long as the patient continues to receive a clinically determined benefit, which likely will be from at least many months to multiple years.

### Combination Therapy

In some cases, a method of the present disclosure for treating cancer in an individual comprises: a) administering a TMMP; and b) administering at least one additional therapeutic agent or therapeutic treatment. Suitable additional therapeutic agents include, but are not limited to, a small molecule cancer chemotherapeutic agent, and an immune checkpoint inhibitor. Suitable additional therapeutic treatments include, e.g., radiation, surgery (e.g., surgical resection of a tumor), and the like.

A treatment method of the present disclosure can comprise co-administration of a TMMP and at least one additional therapeutic agent. By “co-administration” is meant that both a TMMP and at least one additional therapeutic agent are administered to an individual, although not necessarily at the same time, in order to achieve a therapeutic effect that is the result of having administered both the TMMP and the at least one additional therapeutic agent. The administration of the TMMP and the at least one additional therapeutic agent can be substantially simultaneous, e.g., the TMMP can be administered to an individual within about 1 minute to about 24 hours (e.g., within about 1 minute, within about 5 minutes, within about 15 minutes, within about 30 minutes, within about 1 hour, within about 4 hours, within about 8 hours, within about 12 hours, or within about 24 hours) of administration of the at least one additional therapeutic agent. In some cases, a TMMP of the present disclosure is administered to an individual who is undergoing treatment with, or who has undergone treatment with, the at least one additional therapeutic agent. The administration of the TMMP can occur at different times and/or at different frequencies.

As an example, a treatment method of the present disclosure can comprise co-administration of a TMMP and an immune checkpoint inhibitor such as an antibody specific for an immune checkpoint. By “co-administration” is meant that both a TMMP and an immune checkpoint inhibitor (e.g., an antibody specific for an immune checkpoint polypeptide) are administered to an individual, although not necessarily at the same time, in order to achieve a therapeutic effect that is the result of having administered both the TMMP and the immune checkpoint inhibitor (e.g., an antibody specific for an immune checkpoint polypeptide). The administration of the TMMP and the immune checkpoint inhibitor (e.g., an antibody specific for an immune checkpoint polypeptide) can be substantially simultaneous, e.g., the TMMP can be administered to an individual within about 1 minute to about 24 hours (e.g., within about 1 minute, within about 5 minutes, within about 15 minutes, within about 30 minutes, within about 1 hour, within about 4 hours, within about 8 hours, within about 12 hours, within about 24 hours, within 1 week, 3 weeks 3 weeks, four weeks or a month following administration of the immune checkpoint inhibitor (e.g., an antibody specific for an immune checkpoint polypeptide). The TMMP In some cases, a TMMP of the present disclosure is administered to an individual who is undergoing treatment with, or who has undergone treatment with, an immune checkpoint inhibitor (e.g., an antibody specific for an immune checkpoint polypeptide). The administration of the TMMP and the immune checkpoint inhibitor (e.g., an antibody specific for an immune checkpoint polypeptide) can occur at different times and/or at different frequencies. Where there is an established dosing interval for the checkpoint inhibitor, depending on the interval, it may be possible to administer the TMMP on the same day as the checkpoint inhibitor. For example, in some cases, where the dosing schedule for pembrolizumab is once every three weeks, the pharmaceutical composition comprising the TMMP may be administered on the same day. Patients who receive such combination treatment can have any CPS score (e.g., from 0 to 20, up to 30, up to 50, or greater than 50), including patients who have a CPS score of 20 or less (e.g., 15 or below or 10 or below), which may be generally indicative of a patient who is unlikely to benefit from receiving an anti-PD1 checkpoint inhibitor alone. In some cases, the combination of the TMMP is given to patients whose CPS score is 20 or less.

Exemplary immune checkpoint inhibitors include inhibitors that target an immune checkpoint polypeptide such as CD27, CD28, CD40, CD122, CD96, CD73, CD47, OX40, GITR, CSF1R, JAK, PI3K delta, PI3K gamma, TAM, arginase, CD137 (also known as 4-1BB), ICOS, A2AR, B7-H3, B7-H4, BTLA, CTLA-4, LAG3, TIM3, VISTA, CD96, TIGIT, CD122, PD-1, PD-L1 and PD-L2. In some cases, the immune checkpoint polypeptide is a stimulatory checkpoint molecule selected from CD27, CD28, CD40, ICOS, OX40, GITR, CD122 and CD137. In some cases, the immune checkpoint polypeptide is an inhibitory checkpoint molecule selected from A2AR, B7-H3, B7-H4, BTLA, CTLA-4, IDO, KIR, LAG3, PD-1, TIM3, CD96, TIGIT and VISTA.

In some cases, the immune checkpoint inhibitor is an antibody specific for an immune checkpoint polypeptide. In some cases, the anti-immune checkpoint antibody is a monoclonal antibody. In some cases, the anti-immune checkpoint antibody is humanized, or de-immunized such that the antibody does not substantially elicit an immune response in a human. In some cases, the anti-immune checkpoint antibody is a humanized monoclonal antibody. In some cases, the anti-immune checkpoint antibody is a de-immunized monoclonal antibody. In some cases, the anti-immune checkpoint antibody is a fully human monoclonal antibody. In some cases, the anti-immune checkpoint antibody inhibits binding of the immune checkpoint polypeptide to a ligand for the immune checkpoint polypeptide. In some cases, the anti-immune checkpoint antibody inhibits binding of the immune checkpoint polypeptide to a receptor for the immune checkpoint polypeptide.

Suitable anti-immune checkpoint antibodies include, but are not limited to, nivolumab (Bristol-Myers Squibb), pembrolizumab (Merck), pidilizumab (Curetech), AMP-224 (GlaxoSmithKline/Amplimmune), MPDL3280A (Roche), MDX-1105 (Medarex, Inc./Bristol Myer Squibb), MEDI-4736 (Medimmune/AstraZeneca), arelumab (Merck Serono), ipilimumab (YERVOY, (Bristol-Myers Squibb), tremelimumab (Pfizer), pidilizumab (CureTech, Ltd.), IMP321 (Immutep S.A.), MGA271 (Macrogenics), BMS-986016 (Bristol-Meyers Squibb), lirilumab (Bristol-Myers Squibb), urelumab (Bristol-Meyers Squibb), PF-05082566 (Pfizer), IPH2101 (Innate Pharma/Bristol-Myers Squibb), MEDI-6469 (Medlmmune/AZ), CP-870,893 (Genentech), Mogamulizumab (Kyowa Hakko Kirin), Varlilumab (CelIDex Therapeutics), Avelumab (EMD Serono), Galiximab (Biogen Idec), AMP-514 (Amplimmune/AZ), AUNP 12 (Aurigene and Pierre Fabre), Indoximod (NewLink Genetics), NLG-919 (NewLink Genetics), INCB024360 (Incyte); KN035; and combinations thereof. For example, in some cases, the immune checkpoint inhibitor is an anti-PD-1 antibody. Suitable anti-PD-1 antibodies include, e.g., nivolumab, pembrolizumab (also known as MK-3475), cemiplimab (Libtayo®), pidilizumab, SHR-1210, PDR001, and AMP-224. In some cases, the anti-PD-1 monoclonal antibody is nivolumab, pembrolizumab or PDR001. In some cases, the anti-PD-1 monoclonal antibody is cemiplimab. Suitable anti-PD1 antibodies are described in U.S. Patent Publication No. 2017/0044259. For pidilizumab, see, e.g., Rosenblatt et al. (2011) J. Immunother. 34:409-18. In some cases, the immune checkpoint inhibitor is an anti-CTLA-4 antibody. In some cases, the anti-CTLA-4 antibody is ipilimumab or tremelimumab. For tremelimumab, see, e.g., Ribas et al. (2013) J. Clin. Oncol. 31:616-22. In some cases, the immune checkpoint inhibitor is an anti-PD-L1 antibody. In some cases, the anti-PD-L1 monoclonal antibody is BMS-935559, MEDI4736, MPDL3280A (also known as RG7446), KN035, or MSB0010718C. In some embodiments, the anti-PD-L1 monoclonal antibody is MPDL3280A (atezolizumab) or MEDI4736 (durvalumab). For durvalumab, see, e.g., WO 2011/066389. For atezolizumab, see, e.g., U.S. Pat. No. 8,217,149. In some cases, the immune checkpoint inhibitor is an anti-TIGIT antibody that binds to T-cell immunoreceptor with immunoglobulin and ITIM domains (TIGIT). In some cases, the anti-TIGIT antibody is BMS-986207 (Bristol-Myers Squibb). In some cases, the anti-TIGIT antibody is tiragolumab. In some cases, the anti-TIGIT antibody is EOS88448 (EOS-448). See, e.g., U.S. Pat. Nos. 11,008,390 and 10,189,902; U.S. Patent Publication No. 2017/0088613; and WO 2019/137541.

Among such checkpoint inhibitors, antibodies to PD-1, PD-L1 and CTLA-4 are the most common, with at least nivolumab, tremelimumab, pembrolizumab, ipilimumab, cemiplimab, atezolizumab, avelumab, tisleizumab and durvalumab having been approved by the FDA and/or regulatory agencies outside of the U.S. A TMMP of this disclosure also may be co-administered with combinations of checkpoint inhibitors, e.g., a combination of (i) an antibody to PD-1 or PD-L1, and (ii) an antibody to CTLA-4.

### Subjects Suitable for Treatment

Subjects suitable for treatment with a method include individuals who have cancer, including individuals who have been diagnosed as having cancer, individuals who have been treated for cancer but who failed to respond to the treatment, and individuals who have been treated for cancer and who initially responded but subsequently became refractory to the treatment and/or whose disease progressed while on the prior treatment. Such subjects can include individuals whose cancers exhibit HLA loss. Such subjects can include individuals whose CPS score is 20 or less.

In some cases, the subject is an individual who is undergoing treatment with an immune checkpoint inhibitor. In some cases, the subject is an individual who has undergone treatment with an immune checkpoint inhibitor, but whose disease has progressed despite having received such treatment. In some cases, the subject is an individual who is undergoing treatment with, or who has undergone treatment with, a cancer chemotherapeutic agent. In some cases, the subject is an individual who is preparing to undergo treatment with, is undergoing treatment with, or who has undergone treatment with, an immune checkpoint inhibitor. In some cases, the subject is an individual who is preparing to undergo treatment with, is undergoing treatment with, or who has undergone treatment with, a cancer chemotherapeutic agent, radiation treatment, surgery, and/or treatment with another therapeutic agent. In some cases, a pharmaceutical composition comprising the TMMP is administered in the adjuvant or neoadjuvant setting.

### Examples of Non-Limiting Aspects of the Disclosure

### Aspects Set A

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. A T-cell modulatory multimeric polypeptide comprising: at least one heterodimer comprising: a) a first polypeptide comprising: i) a SARS-CoV-2 peptide, wherein the SARS-CoV-2 peptide has a length of from about 4 amino acids to about 25 amino acids; and ii) first major histocompatibility complex (MHC) polypeptide, wherein the first MHC polypeptide is a β2 microglobulin (β2M) polypeptide; b) a second polypeptide comprising a second MHC polypeptide, wherein the second MHC polypeptide is an MHC class I heavy chain polypeptide; c) at least one immunomodulatory polypeptide, wherein the first and/or the second polypeptide comprises the at least one immunomodulatory polypeptide; d) an immunoglobulin (Ig) Fc polypeptide or a non-Ig scaffold, wherein the first or the second polypeptide comprises the Ig Fc polypeptide or the non-Ig scaffold; and e) a tumor-targeting polypeptide (TTP), wherein the first and/or the second polypeptide comprises the TTP, optionally wherein one or more of the individual components of the first and/or second polypeptide in each of the above TMMP combinations are linked together by one or more linkers.

Aspect 2. A TMMP of aspect 1, wherein
a1) the first polypeptide comprises, in order from N-terminus to C-terminus:
   i) the SARS-CoV-2 peptide; and
   ii) the β2M polypeptide; and
b1) the second polypeptide comprises, in order from N-terminus to C-terminus:
   i) the TTP;
   ii) the at least one immunomodulatory polypeptide;
   iii) the MHC class I heavy chain polypeptide; and
   iv) an Ig Fc polypeptide; or
a2) the first polypeptide comprises, in order from N-terminus to C-terminus:
   i) the SARS-CoV-2 peptide; and
   ii) the β2M polypeptide; and
b2) the second polypeptide comprises, in order from N-terminus to C-terminus:
   i) the TTP;
   ii) the MHC class I heavy chain polypeptide;
   iii) the at least one immunomodulatory polypeptide; and
   iv) an Ig Fc polypeptide; or
a3) the first polypeptide comprises, in order from N-terminus to C-terminus:
   i) the SARS-CoV-2 peptide; and
   ii) the β2M polypeptide; and
b3) the second polypeptide comprises, in order from N-terminus to C-terminus:
   i) the TTP;
   ii) the MHC class I heavy chain polypeptide;
   iii) an Ig Fc polypeptide; and
   iv) the at least one immunomodulatory polypeptide; or
a4) the first polypeptide comprises, in order from N-terminus to C-terminus:
   i) the at least one immunomodulatory polypeptide;
   ii) the SARS-CoV-2 peptide; and
   iii) the β2M polypeptide; and
b4) the second polypeptide comprises, in order from N-terminus to C-terminus:
   i) the TTP;
   ii) the MHC class I heavy chain polypeptide; and
   iii) an Ig Fc polypeptide; or
a5) the first polypeptide comprises, in order from N-terminus to C-terminus:
   i) the SARS-CoV-2 peptide;
   ii) the β2M polypeptide; and
   iii) the at least one immunomodulatory polypeptide; and
b5) the second polypeptide comprises, in order from N-terminus to C-terminus:
   i) the TTP;
   ii) the MHC class I heavy chain; and
   iii) an Ig Fc polypeptide; or
a6) the first polypeptide comprises, in order from N-terminus to C-terminus:
   i) the SARS-CoV-2 peptide; and
   ii) the β2M polypeptide; and
b6) the second polypeptide comprises, in order from N-terminus to C-terminus:
   i) the at least one immunomodulatory polypeptide;
   ii) the MHC class I heavy chain polypeptide;
   iii) an Ig Fc polypeptide; and
   iv) the TTP; or
a7) the first polypeptide comprises, in order from N-terminus to C-terminus:
   i) the SARS-CoV-2 peptide; and
   ii) the β2M polypeptide; and
b7) the second polypeptide comprises, in order from N-terminus to C-terminus:
   i) the MHC class I heavy chain polypeptide;
   ii) the at least one immunomodulatory polypeptide;
   iii) an Ig Fc polypeptide; and
   iv) the TTP; or
a8) the first polypeptide comprises, in order from N-terminus to C-terminus:
   i) the SARS-CoV-2 peptide; and
   ii) the β2M polypeptide; and
b8) the second polypeptide comprises, in order from N-terminus to C-terminus:
   i) the MHC class I heavy chain polypeptide;
   ii) an Ig Fc polypeptide;
   iii) the at least one immunomodulatory polypeptide; and
   iv) the TTP; or
a9) the first polypeptide comprises, in order from N-terminus to C-terminus:
   i) the at least one immunomodulatory polypeptide;
   ii) the SARS-CoV-2 peptide; and
   iii) the β2M polypeptide; and
b9) the second polypeptide comprises, in order from N-terminus to C-terminus:
   i) the MHC class I heavy chain polypeptide;
   ii) an Ig Fc polypeptide; and
   iii) the TTP; or
a10) the first polypeptide comprises, in order from N-terminus to C-terminus:
   i) the SARS-CoV-2 peptide;
   ii) the β2M polypeptide; and
   iii) the at least one immunomodulatory polypeptide; and
b10) the second polypeptide comprises, in order from N-terminus to C-terminus:
   i) the MHC class I heavy chain;
   ii) an Ig Fc polypeptide; and
   iii) the TTP; or
a11) the first polypeptide comprises, in order from N-terminus to C-terminus:
   i) the SARS-CoV-2 peptide;
   ii) the β2M polypeptide; and
   iii) the TTP; and
b11) the second polypeptide comprises, in order from N-terminus to C-terminus:
   i) the at least one immunomodulatory polypeptide;
   ii) the MHC class I heavy chain polypeptide; and
   iii) an Ig Fc polypeptide; or
a12) the first polypeptide comprises, in order from N-terminus to C-terminus:
   i) the SARS-CoV-2 peptide;
   ii) the β2M polypeptide; and
   iii) the TTP; and
b12) the second polypeptide comprises, in order from N-terminus to C-terminus:
   i) the MHC class I heavy chain polypeptide;
   ii) the at least one immunomodulatory polypeptide; and
   iii) an Ig Fc polypeptide; or
a13) the first polypeptide comprises, in order from N-terminus to C-terminus:
   i) the SARS-CoV-2 peptide;
   ii) the β2M polypeptide; and
   iii) the TTP; and
b13) the second polypeptide comprises, in order from N-terminus to C-terminus:
   i) the MHC class I heavy chain polypeptide;
   ii) an Ig Fc polypeptide; and
   iii) the at least one immunomodulatory polypeptide; or
a14) the first polypeptide comprises, in order from N-terminus to C-terminus:
   i) the at least one immunomodulatory polypeptide;
   ii) the SARS-CoV-2 peptide;
   iii) the β2M polypeptide; and
   iv) the TTP; and
b14) the second polypeptide comprises, in order from N-terminus to C-terminus:
   i) the MHC class I heavy chain polypeptide; and
   ii) an Ig Fc polypeptide; or
a15) the first polypeptide comprises, in order from N-terminus to C-terminus:
   i) the SARS-CoV-2 peptide;
   ii) the β2M polypeptide;
   iii) the at least one immunomodulatory polypeptide; and
   iv) the TTP; and
b15) the second polypeptide comprises, in order from N-terminus to C-terminus:
   i) the MHC class I heavy chain; and
   ii) an Ig Fc polypeptide,
optionally wherein one or more of the individual components of the first and/or second polypeptide in each of the above TMMP combinations are linked together by one or more linkers.

Aspect 3. A TMMP of aspect 1, wherein
a1) the first polypeptide comprises, in order from N-terminus to C-terminus:
   i) the SARS-CoV-2 peptide; and
   ii) the β2M polypeptide; and
b1) the second polypeptide comprises, in order from N-terminus to C-terminus:
   i) the TTP;
   ii) the at least one immunomodulatory polypeptide;
   iii) the MHC class I heavy chain polypeptide; and
   iv) an Ig Fc polypeptide; or
a2) the first polypeptide comprises, in order from N-terminus to C-terminus:
   i) the SARS-CoV-2 peptide; and
   ii) the β2M polypeptide; and
b2) the second polypeptide comprises, in order from N-terminus to C-terminus:
   i) the TTP;
   ii) the MHC class I heavy chain polypeptide;
   iii) the Ig Fc polypeptide; and
   iv) the one or more immunomodulatory polypeptides; or
a3) the first polypeptide comprises, in order from N-terminus to C-terminus:
   i) the SARS-CoV-2 peptide; and
   ii) the β2M polypeptide; and
b3) the second polypeptide comprises, in order from N-terminus to C-terminus:
   i) the TTP;
   ii) the MHC class I heavy chain polypeptide;
   iii) the one or more immunomodulatory polypeptides; and
   iv) the Ig Fc polypeptide,
optionally wherein one or more of the individual components of the first and/or second polypeptide in each of the above TMMP combinations are linked together by one or more linkers.

Aspect 4. A TMMP of any one of aspects 1-3, wherein
the first polypeptide comprises, in order from N-terminus to C-terminus:
   i) the SARS-CoV-2 peptide;
   ii) a linker; and
   iii) the β2M polypeptide; and
the second polypeptide comprises, in order from N-terminus to C-terminus:
   i) the TTP;
   ii) a linker;
   iii) the MHC class I heavy chain polypeptide;
   iv) a linker;
   v) the Ig Fc polypeptide;
   vi) a linker; and
   vii) the one or more immunomodulatory polypeptides,
wherein when the TMMP comprises more than one immunomodulatory polypeptide, the immunomodulatory polypeptides are linked together by one or more linkers.

Aspect 5. A TMMP of any one of aspects 1-4, wherein at least one of the one or more immunomodulatory polypeptides is a variant immunomodulatory polypeptide that exhibits reduced affinity to a cognate co-immunomodulatory polypeptide compared to the affinity of a corresponding wild-type immunomodulatory polypeptide for the cognate co-immunomodulatory polypeptide.

Aspect 6. A TMMP of aspect 5, wherein at least one of the one or more immunomodulatory polypeptides is a variant IL-2 polypeptide comprising: a) H16A and F42A substitutions; or b) H16T and F42A substitutions.

Aspect 7. A TMMP of any one of aspects 1-6, wherein the second polypeptide comprises an Ig Fc polypeptide, and wherein the Ig Fc polypeptide is an IgG1 Fc polypeptide.

Aspect 8. A TMMP of aspect 7, wherein the IgG1 Fc comprises one or more amino acid substitutions that reduce or substantially eliminate antibody-dependent cell-mediated cytotoxicity (ADCC) and/or complement-dependent cytotoxicity (CDC).

Aspect 9. A TMMP of aspect 8, wherein IgG1 Fc polypeptide comprises L234A and L235A substitutions.

Aspect 10. A TMMP of aspect 9, wherein at least one of the one or more linkers comprises the amino acid sequence (GGGGS)n (SEQ ID NO:934), where n is an integer from 1 to 10.

Aspect 11. A TMMP of any one of aspects 1-10, wherein the TMMP comprises at least two immunomodulatory polypeptides, and wherein at least two of the immunomodulatory polypeptides are the same.

Aspect 12. A TMMP of aspect 11, wherein the two or more immunomodulatory polypeptides are in tandem.

Aspect 13. A TMMP of aspect 12, wherein the two or more immunomodulatory polypeptides are separated by a linker.

Aspect 14. A TMMP of any one of aspects 1-13, wherein the TTP is a single-chain Fv (scFv) antibody or a nanobody.

Aspect 15. A TMMP of any one of aspects 1-14, wherein the TTP binds an antigen selected from mesothelin, TROP-2, Her2, CD19, WT1, MUC1, BCMA, PSMA, B7-H3, CEACAM, CD20, CD22, CD30, CD38, CD138, and a claudin polypeptide.

Aspect 16. A TMMP of any one of aspects 1-15, wherein the TTP binds a CD19 antigen.

Aspect 17. A TMMP of any one of aspects 1-16, wherein the SARS-CoV-2 peptide has a length of from about 8 amino acids to about 12 amino acids.

Aspect 18. A TMMP of any one of aspects 1-17, wherein the SARS-CoV-2 peptide comprises from 4 to 25 contiguous amino acids of a SARS-CoV-2 surface glycoprotein.

Aspect 19. A TMMP of aspect 18, wherein the SARS-CoV-2 peptide is a surface glycoprotein peptide depicted in FIG. 13J, optionally wherein the SARS-CoV-2 peptide is selected from the group consisting of STQDLFLPFF (SEQ ID NO:845), FCNDPFLGVYY (SEQ ID NO:846), SSANNCTFEY (SEQ ID NO:847), STECSNLLLQY (SEQ ID NO:848), YTNSFTRGVY (SEQ ID NO:849), CVADYSVLY (SEQ ID NO:850), LTDEMIAQY (SEQ ID NO:851), KIADYNYKL (SEQ ID NO:219), KLPDDFTGCV (SEQ ID NO:852), SFELLHAPATV (SEQ ID NO:853), LLFNKVTLA (SEQ ID NO:221), VLNDILSRL (SEQ ID NO:222), RLDKVEAEV (SEQ ID NO:223), RLQSLQTYV (SEQ ID NO:224), HLMSFPQSA (SEQ ID NO:225), RLNEVAKNL (SEQ ID NO:854), NLNESLIDL (SEQ ID NO:855), FIAGLIAIV (SEQ ID NO:226), VVFLHVTYV (SEQ ID NO:856), YLQPRTFLL (SEQ ID NO:218), SIIAYTMSL (SEQ ID NO:220), TLDSKTQSL (SEQ ID NO:534), KCYGVSPTK (SEQ ID NO:857), TVYDPLQPELDSFK (SEQ ID NO:333), GVYFASTEK (SEQ ID NO:289), GTHWFVTQR (SEQ ID NO:332), YYVGYLQPRTF (SEQ ID NO:392), NYNYLYRLF (SEQ ID NO:394), YFPLQSYGF (SEQ ID NO:395), VYDPLQPELDSF (SEQ ID NO:858), YEQYIKWPWYI (SEQ ID NO:859), KWPWYIWLGF (SEQ ID NO:860), GTITSGWTF (SEQ ID NO:861), QYIKWPWYI (SEQ ID NO:400), RFDNPVLPF (SEQ ID NO:389), EILDITPCSF (SEQ ID NO:862), FVFKNIDGY (SEQ ID NO:592), SVASQSIIAY (SEQ ID NO:863), WTAGAAAYY (SEQ ID NO:145), VASQSIIAY (SEQ ID NO:597), TPINLVRDL (SEQ ID NO:476), SPRRARSVA (SEQ ID NO:481), APHGVVFLHV (SEQ ID NO:864), LPQGFSAL (SEQ ID NO:477), QPYRVVVL (SEQ ID NO:479), MIAQYTSAL (SEQ ID NO:865), FPQSAPHGVVF (SEQ ID NO:600), SEPVLKGVKL (SEQ ID NO:484), TEKSNIIRGW (SEQ ID NO:711), TECSNLLLQY (SEQ ID NO:866), and REGVFVSNGTHW (SEQ ID NO:715).

Aspect 20. A TMMP any one of aspects 1-19, wherein the SARS-CoV-2 peptide has the sequence YLQPRTFLL (SEQ ID NO:218) and has a length of 9 amino acids.

Aspect 21. A TMMP of any one of aspects 1-19, wherein the first polypeptide and the second polypeptide are covalently linked to one another via one or more disulfide bonds.

Aspect 22. A TMMP of aspect 21, wherein the β2M polypeptide and the MHC heavy chain polypeptide are joined by a disulfide bond that joins a Cys residue in the β2M polypeptide and a Cys residue in the MHC heavy chain polypeptide.

Aspect 23. A TMMP of aspect 22, wherein a Cys at amino acid residue 12 of the β2M polypeptide is disulfide bonded to a Cys at amino acid residue 236 of the MHC heavy chain polypeptide.

Aspect 24. A TMMP of aspect 21, wherein the first polypeptide chain comprises a linker between the peptide epitope and the β2M polypeptide, and wherein the disulfide bond links a Cys present in the linker with a Cys at position 84 of the MHC heavy chain polypeptide.

Aspect 25. A TMMP of any one of aspects 1-24, wherein the first and the second polypeptides are covalently linked to one another via at least 2 disulfide bonds.

Aspect 26. A TMMP of aspect 25, wherein:
a) a first disulfide bond is between: i) a Cys present in a linker between the peptide epitope and the β2M polypeptide; and ii) a Cys residue at position 84 in the MHC class I heavy chain polypeptide; and
b) a second disulfide bond is between: i) a Cys residue at position 12 in the β2M polypeptide; and ii) a Cys residue at position 236 in the MHC Class I heavy chain polypeptide.

Aspect 27. A TMMP of aspect 26, wherein the linker comprises the amino acid sequence GCGGS (SEQ ID NO:977).

Aspect 28. A TMMP of aspect 27, wherein the linker comprises the amino acid sequence GCGGS(GGGGS)n (SEQ ID NO:978), where n is an integer from 1 to 10.

Aspect 29. A TMMP of any one of aspects 1-28, wherein the MHC class I heavy chain is an HLA-E allele heavy chain polypeptide.

Aspect 30. A TMMP of any one of aspects 1-29, wherein the second polypeptide comprises a linker between the TTP and the MHC class I heavy chain, wherein the linker has a length of less than about 25 amino acids, less than about 20 amino acids, less than about 15 amino acids, less than about 10 amino acids, less than 8 amino acids, or less than 6 amino acids.

Aspect 31. A TMMP of aspect 30, wherein the linker has the amino acid sequence GGGGS (SEQ ID NO:873).

Aspect 32. A homodimer comprising two heterodimers according to any one of aspects 1-31, wherein the two heterodimers are covalently linked to one another by one or more disulfide bonds between Ig Fc polypeptides present in the first and second heterodimers.

Aspect 33. A pharmaceutical composition comprising a homodimer of aspect 32.

Aspect 34. A nucleic acid comprising a nucleotide sequence encoding a first and/or second polypeptide according to any one of aspects 1-31.

Aspect 35. An expression vector comprising the nucleic acid of aspect 34.

Aspect 36. A method of selectively modulating the activity of T cell specific for an epitope, the method comprising contacting the T cell with a T-cell modulatory multimeric polypeptide according to any one of aspects 1-31 or a homodimer according to aspect 32, wherein said contacting selectively modulates the activity of the epitope-specific T cell.

Aspect 37. A method of treating a patient having a cancer, the method comprising administering to the patient an effective amount of a pharmaceutical composition comprising T-cell modulatory multimeric polypeptide according to any one of aspects 1-31 or a pharmaceutical composition according to aspect 33.

Aspect 38. The method of aspect 37, wherein the cancer is a carcinoma, sarcoma, melanoma, leukemia, or lymphoma.

Aspect 39. The method of aspect 37 or 38, wherein said administering is intramuscular, intravenous, peritumoral, or intratumoral.

Aspect 40. The method of any one of aspects 37-39, wherein the method comprises administering a SARS-CoV-2 vaccine to the patient prior to administering to the patient an effective amount of a pharmaceutical composition comprising T-cell modulatory multimeric polypeptide.

Aspect 41. The method of any one of aspects 37-39, comprising co-administering an immune checkpoint inhibitor to the patient, optionally wherein the immune checkpoint inhibitor is an antibody specific for PD-L1, PD-1, or CTLA4.

Aspect 42. A protein comprising: at least one heterodimer comprising: a) a first polypeptide comprising: i) a SARS-CoV-2 peptide, wherein the SARS-CoV-2 peptide has a length of from about 4 amino acids to about 25 amino acids; and ii) first major histocompatibility complex (MHC) polypeptide, wherein the first MHC polypeptide is a β2 microglobulin (β2M) polypeptide; b) a second polypeptide comprising a second MHC polypeptide, wherein the second MHC polypeptide is an MHC class I heavy chain polypeptide; c) an immunoglobulin (Ig) Fc polypeptide or a non-Ig scaffold, wherein the first or the second polypeptide comprises the Ig Fc polypeptide or the non-Ig scaffold; and e) a tumor-targeting polypeptide (TTP), wherein the first and/or the second polypeptide comprises the TTP, optionally wherein one or more of the individual components of the first and/or second polypeptide in each of the above combinations are linked together by one or more linkers.

Aspect 43. A homodimer comprising two heterodimers according to aspect 42, wherein the two heterodimers are covalently linked to one another by one or more disulfide bonds between Ig Fc polypeptides present in the first and second heterodimers.

Aspect 44. A T-cell modulatory multimeric polypeptide (TMMP) comprising:
at least one heterodimer comprising:
a) a first polypeptide comprising:
   i) a peptide epitope having a length of from about 4 amino acids to about 25 amino acids; and
   ii) first major histocompatibility complex (MHC) polypeptide, wherein the first MHC polypeptide is a β2 microglobulin (β2M) polypeptide;
b) a second polypeptide comprising a second MHC polypeptide, wherein the second MHC polypeptide is an MHC class I heavy chain polypeptide;
c) an immunoglobulin (Ig) Fc polypeptide or a non-Ig scaffold, wherein the first or the second polypeptide comprises the Ig Fc polypeptide or the non-Ig scaffold;
d) a tumor-targeting polypeptide (TTP), wherein the first and/or the second polypeptide comprises the TTP;
wherein the TMMP does not include an immunomodulatory polypeptide, and
optionally wherein one or more of the individual components of the first and/or second polypeptide in each of the above TMMP combinations are linked together by one or more linkers.

Aspect 45. A TMMP of aspect 44, wherein
a1) the first polypeptide comprises, in order from N-terminus to C-terminus:
   i) the peptide epitope; and
   ii) the β2M polypeptide; and
b1) the second polypeptide comprises, in order from N-terminus to C-terminus:
   i) the TTP;
   ii) the MHC class I heavy chain polypeptide; and
   iv) an Ig Fc polypeptide; or
a2) the first polypeptide comprises, in order from N-terminus to C-terminus:
   i) the peptide epitope; and
   ii) the β2M polypeptide; and
b2) the second polypeptide comprises, in order from N-terminus to C-terminus:
   i) the MHC class I heavy chain polypeptide;
   ii) an Ig Fc polypeptide; and
   iii) the TTP; or
a3) the first polypeptide comprises, in order from N-terminus to C-terminus:
   i) the peptide epitope; and
   ii) the β2M polypeptide; and
b3) the second polypeptide comprises, in order from N-terminus to C-terminus:
   i) the MHC class I heavy chain polypeptide;
   ii) the TTP; and
   iii) an Ig Fc polypeptide.;
   ii) the β2M polypeptide; and
optionally wherein one or more of the individual components of the first and/or second polypeptide in each of the above TMMP combinations are linked together by one or more linkers.

Aspect 46. A TMMP of aspect 44 or aspect 45, wherein
the first polypeptide comprises, in order from N-terminus to C-terminus:
   i) the peptide epitope;
   ii) a linker; and
   iii) the β2M polypeptide; and
the second polypeptide comprises, in order from N-terminus to C-terminus:
   i) the TTP;
   ii) a linker;
   iii) the MHC class I heavy chain polypeptide;
   iv) a linker; and
   v) the Ig Fc polypeptide.

Aspect 47. A TMMP of any one of aspects 44-46, wherein the second polypeptide comprises an Ig Fc polypeptide, and wherein the Ig Fc polypeptide is an IgG1 Fc polypeptide.

Aspect 48. A TMMP of aspect 47, wherein the IgG1 Fc comprises one or more amino acid substitutions that reduce or substantially eliminate antibody-dependent cell-mediated cytotoxicity (ADCC) and/or complement-dependent cytotoxicity (CDC).

Aspect 49. A TMMP of aspect 48, wherein IgG1 Fc polypeptide comprises L234A and L235A substitutions, wherein L234 and L235 correspond to amino acids 14 and 15 of the amino acid sequence depicted in FIG. 2A.

Aspect 50. A TMMP of aspect 46, wherein at least one of the one or more linkers comprises the amino acid sequence (GGGGS)n (SEQ ID NO:934), where n is an integer from 1 to 10.

Aspect 51. A TMMP of any one of aspects 44-50, wherein the TTP is a single-chain Fv (scFv) antibody or a nanobody.

Aspect 52. A TMMP of any one of aspects 44-51, wherein the TTP binds an antigen selected from mesothelin, TROP-2, Her2, CD19, WT1, MUC1, BCMA, PSMA, B7-H3, CEACAM, CD20, CD22, CD30, CD38, CD138, and a claudin polypeptide.

Aspect 53. A TMMP of any one of aspects 44-52, wherein the TTP binds a CD19 antigen.

Aspect 54. A TMMP of any one of aspects 44-53, wherein the peptide epitope has a length of from 8 amino acids to 14 amino acids, optionally from 9 amino acids to 12 amino acids.

Aspect 55. A TMMP of any one of aspects 44-54, wherein the peptide epitope comprises from 4 to 25 contiguous amino acids of a SARS-CoV-2 surface glycoprotein.

Aspect 56. A TMMP of aspect 55, wherein the SARS-CoV-2 peptide is a surface glycoprotein peptide depicted in FIG. 13J, optionally wherein the SARS-CoV-2 peptide is selected from the group consisting of STQDLFLPFF (SEQ ID NO:845), FCNDPFLGVYY (SEQ ID NO:846), SSANNCTFEY (SEQ ID NO:847), STECSNLLLQY (SEQ ID NO:848), YTNSFTRGVY (SEQ ID NO:849), CVADYSVLY (SEQ ID NO:850), LTDEMIAQY (SEQ ID NO:851), KIADYNYKL (SEQ ID NO:219), KLPDDFTGCV (SEQ ID NO:852), SFELLHAPATV (SEQ ID NO:853), LLFNKVTLA (SEQ ID NO:221), VLNDILSRL (SEQ ID NO:222), RLDKVEAEV (SEQ ID NO:223), RLQSLQTYV (SEQ ID NO:224), HLMSFPQSA (SEQ ID NO:225), RLNEVAKNL (SEQ ID NO:854), NLNESLIDL (SEQ ID NO:855), FIAGLIAIV (SEQ ID NO:226), VVFLHVTYV (SEQ ID NO:856), YLQPRTFLL (SEQ ID NO:218), SIIAYTMSL (SEQ ID NO:220), TLDSKTQSL (SEQ ID NO:534), KCYGVSPTK (SEQ ID NO:857), TVYDPLQPELDSFK (SEQ ID NO:333), GVYFASTEK (SEQ ID NO:289), GTHWFVTQR (SEQ ID NO:332), YYVGYLQPRTF (SEQ ID NO:392), NYNYLYRLF (SEQ ID NO:394), YFPLQSYGF (SEQ ID NO:395), VYDPLQPELDSF (SEQ ID NO:858), YEQYIKWPWYI (SEQ ID NO:859), KWPWYIWLGF (SEQ ID NO:860), GTITSGWTF (SEQ ID NO:861), QYIKWPWYI (SEQ ID NO:400), RFDNPVLPF (SEQ ID NO:389), EILDITPCSF (SEQ ID NO:862), FVFKNIDGY (SEQ ID NO:592), SVASQSIIAY (SEQ ID NO:863), WTAGAAAYY (SEQ ID NO:145), VASQSIIAY (SEQ ID NO:597), TPINLVRDL (SEQ ID NO:476), SPRRARSVA (SEQ ID NO:481), APHGVVFLHV (SEQ ID NO:864), LPQGFSAL (SEQ ID NO:477), QPYRVVVL (SEQ ID NO:479), MIAQYTSAL (SEQ ID NO:865), FPQSAPHGVVF (SEQ ID NO:600), SEPVLKGVKL (SEQ ID NO:484), TEKSNIIRGW (SEQ ID NO:711), TECSNLLLQY (SEQ ID NO:866), and REGVFVSNGTHW (SEQ ID NO:715).

Aspect 57. A TMMP any one of aspects 44-54, wherein the peptide epitope is a SARS-CoV-2 peptide epitope, and wherein the SARS-CoV-2 peptide has an amino acid sequence selected from YLQPRTFLL (SEQ ID NO:218), YLQPRTLFL (SEQ ID NO:887), YLQPRTLIL (SEQ ID NO:1201), and YLQPRTLVL (SEQ ID NO:1202), wherein the SARS-CoV-2 peptide has a length of 9 amino acids.

Aspect 58. A TMMP of any one of aspects 44-54, wherein the peptide epitope is a SARS-CoV-2 peptide epitope, and wherein the SARS-CoV-2 polypeptide is a non-structural polypeptide, optionally wherein the non-structural polypeptide is NSP13.

Aspect 59. A TMMP of aspect 58, wherein the peptide epitope is a SARS-CoV-2 peptide epitope, and wherein the SARS-CoV-2 peptide has the amino acid sequence VMPLSAPTL (SEQ ID NO:914) and has a length of 9 amino acids.

Aspect 60. A TMMP of any one of aspects 44-54, wherein the peptide epitope is an epitope of a viral antigen other than a SARS-CoV-2 epitope, optionally where in the peptide epitope is a viral epitope of a virus selected from the group of Adenoviruses, Adeno-associated virus, Alphaviruses (Togaviruses), Eastern equine encephalitis virus, Eastern equine encephalomyelitis virus, Venezuelan equine encephalomyelitis vaccine strain TC-83, Western equine encephalomyelitis virus, Arenaviruses, Lymphocytic choriomeningitis virus (non-neurotropic strains), Tacaribe virus complex, Bunyaviruses, Bunyamwera virus, Rift Valley fever virus vaccine strain MP-12, Chikungunya virus, Calciviruses, Coronaviruses, Cowpox virus, Flaviviruses (Togaviruses)—Group B Arboviruses, Dengue virus serotypes 1, 2, 3, and 4, Yellow fever virus vaccine strain 17D, Hepatitis A, B, C, D, and E viruses, the Cytomegalovirus, Epstein Barr virus, Eastern Equine encephalitis virus, Herpes simplex types 1 and 2, Herpes zoster, Human herpesvirus types 6 and 7, hepatitis C virus (HVC), hepatitis B virus (HBV), Influenza viruses types A, B, and C, Papovaviruses, Newcastle disease virus, Measles virus, Mumps virus, Parainfluenza viruses types 1, 2, 3, and 4, polyomaviruses (JC virus, BK virus), Respiratory syncytial virus, Human parvovirus (B 19), Coxsackie viruses types A and B, Echoviruses, Polioviruses, Rhinoviruses, Alastrim (Variola minor virus), Smallpox (Variola major virus), Whitepox Reoviruses, Coltivirus, human Rotavirus, and Orbivirus (Colorado tick fever virus), Rabies virus, Vesicular stomatitis virus, Rubivirus (rubella), Semliki Forest virus, St. Louis encephalitis virus, Venezuelan equine encephalitis virus, Venezuelan equine encephalomyelitis virus, Arenaviruses (a.k.a. South American Hemorrhagic Fever virus), Flexal, Lymphocytic choriomeningitis virus (LCM) (neurotropic strains), Hantaviruses including Hantaan virus, Rift Valley fever virus, Japanese encephalitis virus, Yellow fever virus, Monkeypox virus, Human immunodeficiency virus (HIV) types 1 and 2, Human T cell lymphotropic virus (HTLV) types 1 and 2, Simian immunodeficiency virus (SIV), Vesicular stomatitis virus, Guanarito virus, Lassa fever virus, Junin virus, Machupo virus, Sabia, Crimean-Congo hemorrhagic fever virus, Ebola viruses, Marburg virus, Tick-borne encephalitis virus complex (flavi) including Central European tick-borne encephalitis, Far Eastern tick-borne encephalitis, Hanzalova, Hypr, Kumlinge, Kyasanur Forest disease, Omsk hemorrhagic fever, and Russian Spring Summer encephalitis viruses, Herpesvirus simiae (Herpes B or Monkey B virus), Cercopithecine herpesvirus 1 (Herpes B virus), Equine morbillivirus (Hendra and Hendra-like viruses), Nipah virus, Variola major virus (Smallpox virus), Variola minor virus (Alastrim), African swine fever virus, African horse sickness virus, Akabane virus, Avian influenza virus (highly pathogenic), Blue tongue virus, Camel pox virus, Classical swine fever virus, Cowdria ruminantium (heartwater), Foot and mouth disease virus, Goat pox virus, Japanese encephalitis virus, Lumpy skin disease virus, Malignant catarrhal fever virus, Menangle virus, Newcastle disease virus (VVND), Vesicular stomatitis virus (exotic), and Zika virus.

Aspect 61. A TMMP of aspect 60, where the viral antigen is a cytomegalovirus (CMV) polypeptide.

Aspect 62. A T-cell modulatory multimeric polypeptide of aspect 61, wherein the CMV polypeptide is a CMV pp65 polypeptide.

Aspect 63. A T-cell modulatory multimeric polypeptide of aspect 62, wherein the peptide has the amino acid sequence NLVPMVATV (SEQ ID NO:913) and has a length of 9 amino acids.

Aspect 64. A TMMP of any one of aspects 54-63, wherein the first polypeptide and the second polypeptide are covalently linked to one another via one or more disulfide bonds.

Aspect 65. A TMMP of aspect 64, wherein the β2M polypeptide and the MHC heavy chain polypeptide are joined by a disulfide bond that joins a Cys residue in the β2M polypeptide and a Cys residue in the MHC heavy chain polypeptide.

Aspect 66. A TMMP of aspect 65, wherein a Cys at amino acid residue 12 of the β2M polypeptide is disulfide bonded to a Cys at amino acid residue 236 of the MHC heavy chain polypeptide.

Aspect 67. A TMMP of aspect 64, wherein the first polypeptide chain comprises a linker between the peptide epitope and the β2M polypeptide, and wherein the disulfide bond links a Cys present in the linker with a Cys at position 84 of the MHC heavy chain polypeptide.

Aspect 68. A TMMP of any one of aspects 44-67, wherein the first and the second polypeptides are covalently linked to one another via at least 2 disulfide bonds.

Aspect 69. A TMMP of aspect 68, wherein:
a) a first disulfide bond is between: i) a Cys present in a linker between the peptide epitope and the β2M polypeptide; and ii) a Cys residue at position 84 in the MHC class I heavy chain polypeptide; and
b) a second disulfide bond is between: i) a Cys residue at position 12 in the β2M polypeptide; and ii) a Cys residue at position 236 in the MHC Class I heavy chain polypeptide.

Aspect 70. A TMMP of aspect 69, wherein the linker comprises the amino acid sequence GCGGS (SEQ ID NO:977).

Aspect 71. A TMMP of aspect 70, wherein the linker comprises the amino acid sequence GCGGS(GGGGS)n (SEQ ID NO:978), where n is an integer from 1 to 10.

Aspect 72. A TMMP of any one of aspects 44-71, wherein the MHC class I heavy chain is an HLA-E allele heavy chain polypeptide.

Aspect 73. A TMMP of aspect 57, wherein:
a) the first polypeptide comprises, in order from N-terminus to C-terminus:
   i) the SARS-CoV-2 peptide, wherein the SARS-CoV2 peptide comprises the amino acid sequence YLQPRTFLL (SEQ ID NO:218);
   ii) a linker; and
   iii) the β2M polypeptide, wherein the β2M polypeptide comprises an amino acid sequence having at least 95% amino acid sequence identity to the β2M amino acid sequence depicted in FIG. 32G; and
b) the second polypeptide comprises, in order from N-terminus to C-terminus:
   i) the TTP, wherein the TTP is an anti-CD19 single chain Fv antibody;
   ii) a linker;
   iii) the MHC class I heavy chain polypeptide, wherein the MHC class I heavy chain comprises an amino acid sequence having at least 95% amino acid sequence identity to the amino acid sequence depicted in FIG. 32D;
   iv) a linker; and
   v) the Ig Fc polypeptide, wherein the Ig Fc polypeptide comprises an amino acid sequence having at least 95% amino acid sequence identity to the amino acid sequence depicted in FIG. 32E.

Aspect 74. A TMMP of aspect 73, wherein:
i) the β2M polypeptide comprises a Cys at position 12;
ii) the TTP comprises an amino acid sequence having at least 95% amino acid sequence identity to the amino acid sequence depicted in FIG. 32C;
iii) the MHC class I heavy chain comprises a Cys at position 84 and a Cys at position 236; and
iv) the Ig Fc polypeptide comprises an Ala at position 14 and an Ala at position 15.

Aspect 75. A TMMP of aspect 74, wherein:
a) the first polypeptide comprises an amino acid sequence having at least 95% amino acid sequence identity to the amino acid sequence depicted in FIG. 32B; and
b) the second polypeptide comprises an amino acid sequence having at least 95% amino acid sequence identity to the amino acid sequence depicted in FIG. 33.

Aspect 76. A homodimer comprising two heterodimers according to any one of aspects 44-75, wherein the two heterodimers are covalently linked to one another by one or more disulfide bonds between Ig Fc polypeptides present in the first and second heterodimers.

Aspect 77. A pharmaceutical composition comprising a heterodimer of any one of aspects 44-75 or a homodimer of aspect 76.

Aspect 78. A nucleic acid comprising a nucleotide sequence encoding a first and/or second polypeptide according to any one of aspects 44-76.

Aspect 79. An expression vector comprising the nucleic acid of aspect 78.

Aspect 80. A method of selectively modulating the activity of T cell specific for an epitope, the method comprising contacting the T cell with a T-cell modulatory multimeric polypeptide according to any one of aspects 44-75 or a homodimer according to aspect 74, wherein said contacting selectively modulates the activity of the epitope-specific T cell.

Aspect 81. A method of treating a patient having a cancer, the method comprising administering to the patient an effective amount of a pharmaceutical composition comprising T-cell modulatory multimeric polypeptide according to any one of aspects 44-75 or a homodimer according to aspect 76.

Aspect 82. The method of aspect 81, wherein the cancer is a carcinoma, sarcoma, melanoma, leukemia, or lymphoma.

Aspect 83. The method of aspect 81 or 82, wherein said administering is intramuscular, intravenous, peritumoral, or intratumoral.

Aspect 84. The method of any one of aspects 81-83, wherein the method comprises administering a SARS-CoV-2 vaccine to the patient prior to administering to the patient an effective amount of a pharmaceutical composition comprising T-cell modulatory multimeric polypeptide.

Aspect 85. The method of any one of aspects 81-84, comprising co-administering an immune checkpoint inhibitor to the patient, optionally wherein the immune checkpoint inhibitor is an antibody specific for PD-L1, PD-1, or CTLA4.

Aspect 86. The method of aspect 85, wherein the checkpoint inhibitor is an antibody specific for PD-1, and the patient has a CPS score of 20 or less.

Aspect 87. The method of aspect 81, wherein cancer is a hematological cancer.

Aspect 88. The method of any one of aspects 81-88, wherein the cancer exhibits HLA loss.

### Aspects Set B

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. A T-cell modulatory multimeric polypeptide (TMMP) comprising:
at least one heterodimer comprising:
a) a first polypeptide comprising:
   i) a SARS-CoV-2 peptide or a peptide of another pathogen, wherein the peptide has a length of from about 4 amino acids to about 25 amino acids; and
   ii) first major histocompatibility complex (MHC) polypeptide, wherein the first MHC polypeptide is a β2 microglobulin (β2M) polypeptide;
b) a second polypeptide comprising a second MHC polypeptide, wherein the second MHC polypeptide is an MHC class I heavy chain polypeptide;
c) at least one immunomodulatory polypeptide, wherein the first and/or the second polypeptide comprises the at least one immunomodulatory polypeptide;
d) an immunoglobulin (Ig) Fc polypeptide or a non-Ig scaffold, wherein the first or the second polypeptide comprises the Ig Fc polypeptide or the non-Ig scaffold;
e) a tumor-targeting polypeptide (TTP), wherein the first and/or the second polypeptide comprises the TTP; and
optionally wherein one or more of the individual components of the first and/or second polypeptide in each of the above TMMP combinations are linked together by one or more independently selected peptide linkers.

Aspect 2. A TMMP of aspect 1, wherein
a1) the first polypeptide comprises, in order from N-terminus to C-terminus:
   i) the SARS-CoV-2 peptide or a peptide of another pathogen; and
   ii) the β2M polypeptide; and
b1) the second polypeptide comprises, in order from N-terminus to C-terminus:
   i) the TTP;
   ii) the at least one immunomodulatory polypeptide;
   iii) the MHC class I heavy chain polypeptide; and
   iv) an Ig Fc polypeptide; or
a2) the first polypeptide comprises, in order from N-terminus to C-terminus:
   i) the SARS-CoV-2 peptide or a peptide of another pathogen; and
   ii) the β2M polypeptide; and
b2) the second polypeptide comprises, in order from N-terminus to C-terminus:
   i) the TTP;
   ii) the MHC class I heavy chain polypeptide;
   iii) the at least one immunomodulatory polypeptide; and
   iv) an Ig Fc polypeptide; or
a3) the first polypeptide comprises, in order from N-terminus to C-terminus:
   i) the SARS-CoV-2 peptide or a peptide of another pathogen; and
   ii) the β2M polypeptide; and
b3) the second polypeptide comprises, in order from N-terminus to C-terminus:
   i) the TTP;
   ii) the MHC class I heavy chain polypeptide;
   iii) an Ig Fc polypeptide; and
   iv) the at least one immunomodulatory polypeptide; or
a4) the first polypeptide comprises, in order from N-terminus to C-terminus:
   i) the at least one immunomodulatory polypeptide;
   ii) the SARS-CoV-2 peptide or a peptide of another pathogen; and
   iii) the β2M polypeptide; and
b4) the second polypeptide comprises, in order from N-terminus to C-terminus:
   i) the TTP;
   ii) the MHC class I heavy chain polypeptide; and
   iii) an Ig Fc polypeptide; or
a5) the first polypeptide comprises, in order from N-terminus to C-terminus:
   i) the SARS-CoV-2 peptide or a peptide of another pathogen;
   ii) the β2M polypeptide; and
   iii) the at least one immunomodulatory polypeptide; and
b5) the second polypeptide comprises, in order from N-terminus to C-terminus:
   i) the TTP;
   ii) the MHC class I heavy chain; and
   iii) an Ig Fc polypeptide; or
a6) the first polypeptide comprises, in order from N-terminus to C-terminus:
   i) the SARS-CoV-2 peptide or a peptide of another pathogen; and
   ii) the β2M polypeptide; and
b6) the second polypeptide comprises, in order from N-terminus to C-terminus:
   i) the at least one immunomodulatory polypeptide;
   ii) the MHC class I heavy chain polypeptide;
   iii) an Ig Fc polypeptide; and
   iv) the TTP; or
a7) the first polypeptide comprises, in order from N-terminus to C-terminus:
   i) the SARS-CoV-2 peptide or a peptide of another pathogen; and
   ii) the β2M polypeptide; and
b7) the second polypeptide comprises, in order from N-terminus to C-terminus:
   i) the MHC class I heavy chain polypeptide;
   ii) the at least one immunomodulatory polypeptide;
   iii) an Ig Fc polypeptide; and
   iv) the TTP; or
a8) the first polypeptide comprises, in order from N-terminus to C-terminus:
   i) the SARS-CoV-2 peptide or a peptide of another pathogen; and
   ii) the β2M polypeptide; and
b8) the second polypeptide comprises, in order from N-terminus to C-terminus:
   i) the MHC class I heavy chain polypeptide;
   ii) an Ig Fc polypeptide;
   iii) the at least one immunomodulatory polypeptide; and
   iv) the TTP; or
a9) the first polypeptide comprises, in order from N-terminus to C-terminus:
   i) the at least one immunomodulatory polypeptide;
   ii) the SARS-CoV-2 peptide or a peptide of another pathogen; and
   iii) the β2M polypeptide; and
b9) the second polypeptide comprises, in order from N-terminus to C-terminus:
   i) the MHC class I heavy chain polypeptide;
   ii) an Ig Fc polypeptide; and
   iii) the TTP; or
a10) the first polypeptide comprises, in order from N-terminus to C-terminus:
   i) the SARS-CoV-2 peptide or a peptide of another pathogen;
   ii) the β2M polypeptide; and
   iii) the at least one immunomodulatory polypeptide; and
b10) the second polypeptide comprises, in order from N-terminus to C-terminus:
   i) the MHC class I heavy chain;
   ii) an Ig Fc polypeptide; and
   iii) the TTP; or
a11) the first polypeptide comprises, in order from N-terminus to C-terminus:
   i) the SARS-CoV-2 peptide or a peptide of another pathogen;
   ii) the β2M polypeptide; and
   iii) the TTP; and
b11) the second polypeptide comprises, in order from N-terminus to C-terminus:
   i) the at least one immunomodulatory polypeptide;
   ii) the MHC class I heavy chain polypeptide; and
   iii) an Ig Fc polypeptide; or
a12) the first polypeptide comprises, in order from N-terminus to C-terminus:
   i) the SARS-CoV-2 peptide or a peptide of another pathogen;
   ii) the β2M polypeptide; and
   iii) the TTP; and
b12) the second polypeptide comprises, in order from N-terminus to C-terminus:
   i) the MHC class I heavy chain polypeptide;
   ii) the at least one immunomodulatory polypeptide; and
   iii) an Ig Fc polypeptide; or
a13) the first polypeptide comprises, in order from N-terminus to C-terminus:
   i) the SARS-CoV-2 peptide or a peptide of another pathogen;
   ii) the β2M polypeptide; and
   iii) the TTP; and
b13) the second polypeptide comprises, in order from N-terminus to C-terminus:
   i) the MHC class I heavy chain polypeptide;
   ii) an Ig Fc polypeptide; and
   iii) the at least one immunomodulatory polypeptide; or
a14) the first polypeptide comprises, in order from N-terminus to C-terminus:
   i) the at least one immunomodulatory polypeptide;
   ii) the SARS-CoV-2 peptide or a peptide of another pathogen;
   iii) the β2M polypeptide; and
   iv) the TTP; and
b14) the second polypeptide comprises, in order from N-terminus to C-terminus:
   i) the MHC class I heavy chain polypeptide; and
   ii) an Ig Fc polypeptide; or
a15) the first polypeptide comprises, in order from N-terminus to C-terminus:
   i) the SARS-CoV-2 peptide or a peptide of another pathogen;
   ii) the β2M polypeptide;
   iii) the at least one immunomodulatory polypeptide; and
   iv) the TTP; and
b15) the second polypeptide comprises, in order from N-terminus to C-terminus:
   i) the MHC class I heavy chain; and
   ii) an Ig Fc polypeptide,
optionally wherein one or more of the individual components of the first and/or second polypeptide in each of the above TMMP combinations are linked together by one or more independently selected peptide linkers.

Aspect 3. A TMMP of aspect 1, wherein
a1) the first polypeptide comprises, in order from N-terminus to C-terminus:
   i) the SARS-CoV-2 peptide or a peptide of another pathogen; and
   ii) the β2M polypeptide; and
b1) the second polypeptide comprises, in order from N-terminus to C-terminus:
   i) the TTP;
   ii) the at least one immunomodulatory polypeptide;
   iii) the MHC class I heavy chain polypeptide; and
   iv) an Ig Fc polypeptide; or
a2) the first polypeptide comprises, in order from N-terminus to C-terminus:
   i) the SARS-CoV-2 peptide or a peptide of another pathogen; and
   ii) the β2M polypeptide; and
b2) the second polypeptide comprises, in order from N-terminus to C-terminus:
   i) the TTP;
   ii) the MHC class I heavy chain polypeptide;
   iii) the Ig Fc polypeptide; and
   iv) the one or more immunomodulatory polypeptides; or
a3) the first polypeptide comprises, in order from N-terminus to C-terminus:
   i) the SARS-CoV-2 peptide or a peptide of another pathogen; and
   ii) the β2M polypeptide; and
b3) the second polypeptide comprises, in order from N-terminus to C-terminus:
   i) the TTP;
   ii) the MHC class I heavy chain polypeptide;
   iii) the one or more immunomodulatory polypeptides; and
   iv) the Ig Fc polypeptide,
optionally wherein one or more of the individual components of the first and/or second polypeptide in each of the above TMMP combinations are linked together by one or more independently selected peptide linkers.

Aspect 4. A TMMP of any one of aspects 1-3, wherein
the first polypeptide comprises, in order from N-terminus to C-terminus:
   i) the SARS-CoV-2 peptide or a peptide of another pathogen;
   ii) a peptide linker; and
   iii) the β2M polypeptide; and
the second polypeptide comprises, in order from N-terminus to C-terminus:
   i) the TTP;
   ii) a peptide linker;
   iii) the MHC class I heavy chain polypeptide;
   iv) a peptide linker;
v) the Ig Fc polypeptide;
vi) a peptide linker; and
vii) the one or more immunomodulatory polypeptides,
wherein when the TMMP comprises more than one immunomodulatory polypeptide, the immunomodulatory polypeptides are linked together by one or more independently selected peptide linkers, and optionally wherein the peptide linker between the TTP and the MHC class I heavy chain polypeptide comprises no more than about 12 amino acids, e.g., no more than 10 amino acids in length (e.g., a (GGGGS)₂ (SEQ ID NO:947) linker), no more than 8 amino acids in length, no more than 7 amino acids in length, no more than 6 amino acids in length, or no more than five amino acids in length, (e.g., a GGGGS (SEQ ID NO:873) linker).

Aspect 5. A TMMP of any one of aspects 1-4, wherein at least one of the one or more immunomodulatory polypeptides is a variant immunomodulatory polypeptide that exhibits reduced affinity to a cognate co-immunomodulatory polypeptide compared to the affinity of a corresponding wild-type immunomodulatory polypeptide for the cognate co-immunomodulatory polypeptide.

Aspect 6. A TMMP of aspect 5, wherein at least one of the one or more immunomodulatory polypeptides is a variant IL-2 polypeptide, wherein the variant IL-2 polypeptide binds to IL-2Rα and IL-2RP, and wherein the IL-2 variant exhibits decreased binding to IL-2Rα and decreased binding to IL-2RP, optionally wherein the variant IL-2 comprises: a) H16A and F42A substitutions; or b) H16T and F42A substitutions, and optionally wherein the one or more immunomodulatory polypeptides is a single copy of a variant IL-2 polypeptide.

Aspect 7. A TMMP of any one of aspects 1-6, wherein the second polypeptide comprises an Ig Fc polypeptide, and wherein the Ig Fc polypeptide is an IgG1 Fc polypeptide.

Aspect 8. A TMMP of aspect 7, wherein the IgG1 Fc comprises one or more amino acid substitutions that reduce or substantially eliminate antibody-dependent cell-mediated cytotoxicity (ADCC) and/or complement-dependent cytotoxicity (CDC).

Aspect 9. A TMMP of aspect 8, wherein IgG1 Fc polypeptide comprises L234A and L235A substitutions, wherein L234 and L235 correspond to amino acids 14 and 15 of the amino acid sequence depicted in FIG. 2A.

Aspect 10. A TMMP of aspect 9, wherein at least one of the one or more linkers comprises the amino acid sequence (GGGGS)n (SEQ ID NO:934), where n is an integer from 1 to 10.

Aspect 11. A TMMP of any one of aspects 1-10, wherein the TMMP comprises at least two immunomodulatory polypeptides, and wherein at least two of the immunomodulatory polypeptides are the same.

Aspect 12. A TMMP of aspect 11, wherein the two or more immunomodulatory polypeptides are in tandem.

Aspect 13. A TMMP of aspect 12, wherein the two or more immunomodulatory polypeptides are separated by a peptide linker.

Aspect 14. A TMMP of any one of aspects 1-13, wherein the TTP is a single-chain Fv (scFv) antibody or a nanobody.

Aspect 15. A TMMP of any one of aspects 1-14, wherein the TTP binds an antigen selected from mesothelin, TROP-2, Her2, CD19, WT1, MUC1, BCMA, PSMA, B7-H3, CEACAM, CD20, CD22, CD30, CD38, CD138, and a claudin polypeptide.

Aspect 16. A TMMP of any one of aspects 1-15, wherein the TTP binds a CD19 antigen.

Aspect 17. A TMMP of any one of aspects 1-16, wherein the SARS-CoV-2 peptide or a peptide of another pathogen has a length of from 8 amino acids to 14 amino acids, optionally from 9 amino acids to 12 amino acids.

Aspect 18. A TMMP of any one of aspects 1-16, wherein the SARS-CoV-2 peptide comprises from 4 to 25 contiguous amino acids of a SARS-CoV-2 surface glycoprotein.

Aspect 19. A TMMP of aspect 18, wherein the SARS-CoV-2 peptide is a surface glycoprotein peptide depicted in FIG. 13J, optionally wherein the SARS-CoV-2 peptide is selected from the group consisting of STQDLFLPFF (SEQ ID NO:845), FCNDPFLGVYY (SEQ ID NO:846), SSANNCTFEY (SEQ ID NO:847), STECSNLLLQY (SEQ ID NO:848), YTNSFTRGVY (SEQ ID NO:849), CVADYSVLY (SEQ ID NO:850), LTDEMIAQY (SEQ ID NO:851), KIADYNYKL (SEQ ID NO:219), KLPDDFTGCV (SEQ ID NO:852), SFELLHAPATV (SEQ ID NO:853), LLFNKVTLA (SEQ ID NO:221), VLNDILSRL (SEQ ID NO:222), RLDKVEAEV (SEQ ID NO:223), RLQSLQTYV (SEQ ID NO:224), HLMSFPQSA (SEQ ID NO:225), RLNEVAKNL (SEQ ID NO:854), NLNESLIDL (SEQ ID NO:855), FIAGLIAIV (SEQ ID NO:226), VVFLHVTYV (SEQ ID NO:856), YLQPRTFLL (SEQ ID NO:218), SIIAYTMSL (SEQ ID NO:220), TLDSKTQSL (SEQ ID NO:534), KCYGVSPTK (SEQ ID NO:857), TVYDPLQPELDSFK (SEQ ID NO:333), GVYFASTEK (SEQ ID NO:289), GTHWFVTQR (SEQ ID NO:332), YYVGYLQPRTF (SEQ ID NO:392), NYNYLYRLF (SEQ ID NO:394), YFPLQSYGF (SEQ ID NO:395), VYDPLQPELDSF (SEQ ID NO:858), YEQYIKWPWYI (SEQ ID NO:859), KWPWYIWLGF (SEQ ID NO:860), GTITSGWTF (SEQ ID NO:861), QYIKWPWYI (SEQ ID NO:400), RFDNPVLPF (SEQ ID NO:389), EILDITPCSF (SEQ ID NO:862), FVFKNIDGY (SEQ ID NO:592), SVASQSIIAY (SEQ ID NO:863), WTAGAAAYY (SEQ ID NO:145), VASQSIIAY (SEQ ID NO:597), TPINLVRDL (SEQ ID NO:476), SPRRARSVA (SEQ ID NO:481), APHGVVFLHV (SEQ ID NO:864), LPQGFSAL (SEQ ID NO:477), QPYRVVVL (SEQ ID NO:479), MIAQYTSAL (SEQ ID NO:865), FPQSAPHGVVF (SEQ ID NO:600), SEPVLKGVKL (SEQ ID NO:484), TEKSNIIRGW (SEQ ID NO:711), TECSNLLLQY (SEQ ID NO:866), and REGVFVSNGTHW (SEQ ID NO:715).

Aspect 20. A TMMP any one of aspects 1-19, wherein the SARS-CoV-2 peptide has an amino acid sequence selected from YLQPRTFLL (SEQ ID NO:218), YLQPRTLFL (SEQ ID NO:887), YLQPRTLIL (SEQ ID NO:1201), and YLQPRTLVL (SEQ ID NO:1202), wherein the SARS-CoV-2 peptide has a length of 9 amino acids.

Aspect 21. A TMMP of any one of aspects 1-19, wherein the first polypeptide and the second polypeptide are covalently linked to one another via one or more disulfide bonds.

Aspect 22. A TMMP of aspect 21, wherein the β2M polypeptide and the MHC heavy chain polypeptide are joined by a disulfide bond that joins a Cys residue in the β2M polypeptide and a Cys residue in the MHC heavy chain polypeptide.

Aspect 23. A TMMP of aspect 22, wherein a Cys at amino acid residue 12 of the β2M polypeptide is disulfide bonded to a Cys at amino acid residue 236 of the MHC heavy chain polypeptide.

Aspect 24. A TMMP of aspect 21, wherein the first polypeptide chain comprises a peptide linker between the peptide epitope and the β2M polypeptide, and wherein the disulfide bond links a Cys present in the linker with a Cys at position 84 of the MHC heavy chain polypeptide.

Aspect 25. A TMMP of aspect 4, wherein:
a) the first polypeptide comprises, in order from N-terminus to C-terminus:
   i) the SARS-CoV-2 peptide, wherein the SARS-CoV2 peptide comprises the amino acid sequence YLQPRTFLL (SEQ ID NO:218);
   ii) a peptide linker; and
   iii) the β2M polypeptide, wherein the β2M polypeptide comprises an amino acid sequence having at least 95% amino acid sequence identity to the β2M amino acid sequence depicted in FIG. 32G; and
b) the second polypeptide comprises, in order from N-terminus to C-terminus:
   i) the TTP, wherein the TTP is an anti-CD19 single chain Fv antibody;
   ii) a peptide linker;
   iii) the MHC class I heavy chain polypeptide, wherein the MHC class I heavy chain comprises an amino acid sequence having at least 95% amino acid sequence identity to the amino acid sequence depicted in FIG. 32D;
   iv) a peptide linker;
   v) the Ig Fc polypeptide, wherein the Ig Fc polypeptide comprises an amino acid sequence having at least 95% amino acid sequence identity to the amino acid sequence depicted in FIG. 32E;
   vi) a peptide linker;
   vii) a first immunomodulatory polypeptide, wherein the first immunomodulatory polypeptide comprises an amino acid sequence having at least 95% amino acid sequence identity to the amino acid sequence depicted in FIG. 32F;
   viii) a peptide linker; and
   ix) a second immunomodulatory polypeptide, wherein the second immunomodulatory polypeptide comprises an amino acid sequence having at least 95% amino acid sequence identity to the amino acid sequence depicted in FIG. 32F.

Aspect 26. A TMMP of aspect 25, wherein:
i) the β2M polypeptide comprises a Cys at position 12;
ii) the TTP comprises an amino acid sequence having at least 95% amino acid sequence identity to the amino acid sequence depicted in FIG. 32C;
iii) the MHC class I heavy chain comprises a Cys at position 84 and a Cys at position 236;
iv) the Ig Fc polypeptide comprises an Ala at position 14 and an Ala at position 15; and
v) the first immunomodulatory polypeptide and the second immunomodulatory polypeptide both comprise an Ala at position 16 and an Ala at position 42.

Aspect 27. A TMMP of aspect 26, wherein:
a) the first polypeptide comprises an amino acid sequence having at least 95% amino acid sequence identity to the amino acid sequence depicted in FIG. 32B; and
b) the second polypeptide comprises an amino acid sequence having at least 95% amino acid sequence identity to the amino acid sequence depicted in FIG. 32A.

Aspect 28. A TMMP of aspect 27, wherein:
a) the first polypeptide comprises the amino acid sequence depicted in FIG. 32B; and
b) the second polypeptide comprises the amino acid sequence depicted in FIG. 32A.

Aspect 29. A TMMP of any one of aspects 1-24, wherein the first and the second polypeptides are covalently linked to one another via at least 2 disulfide bonds.

Aspect 30. A TMMP of aspect 25, wherein:
a) a first disulfide bond is between: i) a Cys present in a linker between the peptide epitope and the β2M polypeptide; and ii) a Cys residue at position 84 in the MHC class I heavy chain polypeptide; and
b) a second disulfide bond is between: i) a Cys residue at position 12 in the β2M polypeptide; and ii) a Cys residue at position 236 in the MHC Class I heavy chain polypeptide.

Aspect 31. A TMMP of aspect 30, wherein the linker comprises the amino acid sequence GCGGS (SEQ ID NO:977), or the amino acid sequence GCGGS(GGGGS)n (SEQ ID NO:978), where n is an integer from 1 to 10.

Aspect 32. A TMMP of any one of aspects 1-31, wherein the MHC class I heavy chain is an HLA-E allele heavy chain polypeptide.

Aspect 33. A TMMP of any one of aspects 1-32, wherein the second polypeptide comprises a linker between the TTP and the MHC class I heavy chain, wherein the peptide linker has a length of less than about 25 amino acids, less than about 20 amino acids, less than about 15 amino acids, less than about 10 amino acids, less than about 8 amino acids, or less than about 6 amino acids.

Aspect 34. A TMMP of any one of aspects 4-32, wherein the second polypeptide comprises a linker between the TTP and the MHC class I heavy chain, wherein the peptide linker comprises no more than about 12 amino acids, e.g., no more than 10 amino acids in length (e.g., a (GGGGS)₂ (SEQ ID NO:947) linker), no more than 8 amino acids in length, no more than 7 amino acids in length, no more than 6 amino acids in length, or no more than five amino acids in length, (e.g., a GGGGS (SEQ ID NO:873) linker).

Aspect 35. A TMMP of aspect 34, wherein the peptide linker is no more than five amino acids in length and optionally has the amino acid sequence GGGGS (SEQ ID NO:873), or is no more than 10 amino acids in length and optionally has the amino acid sequence (GGGGS)₂ (SEQ ID NO:947).

Aspect 36. A TMMP of any one of aspects 33-35, wherein the second polypeptide comprises a peptide linker between the Ig Fc polypeptide and one of the at least one immunomodulatory polypeptides, and wherein the peptide linker between the Ig Fc polypeptide and the immunomodulatory polypeptide is (GGGGS)3 (SEQ ID NO:875) or (AP)4 (SEQ ID NO:963).

Aspect 37. A TMMP of aspect 1, wherein:
a1) the first polypeptide comprises an amino acid sequence having at least 95%, at least 98%, at least 99%, or 100%, amino acid sequence identity to the amino acid sequence depicted in FIG. 32B; and
b1) the second polypeptide comprises an amino acid sequence having at least 95%, at least 98%, at least 99%, or 100%, amino acid sequence identity to the amino acid sequence depicted in FIG. 38A;
a2) the first polypeptide comprises an amino acid sequence having at least 95%, at least 98%, at least 99%, or 100%, amino acid sequence identity to the amino acid sequence depicted in FIG. 32B; and
b2) the second polypeptide comprises an amino acid sequence having at least 95%, at least 98%, at least 99%, or 100%, amino acid sequence identity to the amino acid sequence depicted in FIG. 38B;
a3) the first polypeptide comprises an amino acid sequence having at least 95%, at least 98%, at least 99%, or 100%, amino acid sequence identity to the amino acid sequence depicted in FIG. 32B; and
b3) the second polypeptide comprises an amino acid sequence having at least 95%, at least 98%, at least 99%, or 100%, amino acid sequence identity to the amino acid sequence depicted in FIG. 38C;
a4) the first polypeptide comprises an amino acid sequence having at least 95%, at least 98%, at least 99%, or 100%, amino acid sequence identity to the amino acid sequence depicted in FIG. 32B; and
b4) the second polypeptide comprises an amino acid sequence having at least 95%, at least 98%, at least 99%, or 100%, amino acid sequence identity to the amino acid sequence depicted in FIG. 38D;
a5) the first polypeptide comprises an amino acid sequence having at least 95%, at least 98%, at least 99%, or 100%, amino acid sequence identity to the amino acid sequence depicted in FIG. 32B; and
b5) the second polypeptide comprises an amino acid sequence having at least 95%, at least 98%, at least 99%, or 100%, amino acid sequence identity to the amino acid sequence depicted in FIG. 38E;
a6) the first polypeptide comprises an amino acid sequence having at least 95%, at least 98%, at least 99%, or 100%, amino acid sequence identity to the amino acid sequence depicted in FIG. 32B; and
b6) the second polypeptide comprises an amino acid sequence having at least 95%, at least 98%, at least 99%, or 100%, amino acid sequence identity to the amino acid sequence depicted in FIG. 38F;
a7) the first polypeptide comprises an amino acid sequence having at least 95%, at least 98%, at least 99%, or 100%, amino acid sequence identity to the amino acid sequence depicted in FIG. 32B; and
b7) the second polypeptide comprises an amino acid sequence having at least 95%, at least 98%, at least 99%, or 100%, amino acid sequence identity to the amino acid sequence depicted in FIG. 38G;
a8) the first polypeptide comprises an amino acid sequence having at least 95%, at least 98%, at least 99%, or 100%, amino acid sequence identity to the amino acid sequence depicted in FIG. 32B; and
b8) the second polypeptide comprises an amino acid sequence having at least 95%, at least 98%, at least 99%, or 100%, amino acid sequence identity to the amino acid sequence depicted in FIG. 38H.
a9) the first polypeptide comprises an amino acid sequence having at least 95%, at least 98%, at least 99%, or 100%, amino acid sequence identity to the amino acid sequence depicted in FIG. 32B; and
b9) the second polypeptide comprises an amino acid sequence having at least 95%, at least 98%, at least 99%, or 100%, amino acid sequence identity to the amino acid sequence depicted in FIG. 42A;
a10) the first polypeptide comprises an amino acid sequence having at least 95%, at least 98%, at least 99%, or 100%, amino acid sequence identity to the amino acid sequence depicted in FIG. 32B; and
b10) the second polypeptide comprises an amino acid sequence having at least 95%, at least 98%, at least 99%, or 100%, amino acid sequence identity to the amino acid sequence depicted in FIG. 42B; or
a1 l) the first polypeptide comprises an amino acid sequence having at least 95%, at least 98%, at least 99%, or 100%, amino acid sequence identity to the amino acid sequence depicted in FIG. 32B; and
b11) the second polypeptide comprises an amino acid sequence having at least 95%, at least 98%, at least 99%, or 100%, amino acid sequence identity to the amino acid sequence depicted in FIG. 42C.

Aspect 38. A TMMP of aspect 1, wherein:
a1) the first polypeptide comprises an amino acid sequence having at least 95%, at least 98%, at least 99%, or 100%, amino acid sequence identity to the amino acid sequence depicted in FIG. 41B; and
b1) the second polypeptide comprises an amino acid sequence having at least 95%, at least 98%, at least 99%, or 100%, amino acid sequence identity to the amino acid sequence depicted in FIG. 41A.

Aspect 39. A TMMP of aspect 1, wherein:
a1) the first polypeptide comprises an amino acid sequence having at least 95%, at least 98%, at least 99%, or 100%, amino acid sequence identity to the amino acid sequence depicted in FIG. 41C; and
b1) the second polypeptide comprises an amino acid sequence having at least 95%, at least 98%, at least 99%, or 100%, amino acid sequence identity to the amino acid sequence depicted in FIG. 41A.

Aspect 40. A homodimer comprising two heterodimers according to any one of aspects 1-39, wherein the two heterodimers are covalently linked to one another by one or more disulfide bonds between Ig Fc polypeptides present in the first and second heterodimers.

Aspect 41. A pharmaceutical composition comprising a heterodimer of any one of aspects 1-39 or a homodimer of aspect 40.

Aspect 42. A composition comprising:
a) a nucleic acid comprising a nucleotide sequence encoding the first polypeptide and the second polypeptide according to any one of aspects 1-39; or
b) first and second nucleic acids, wherein:
   i) the first nucleic acid comprises a nucleotide sequence encoding the first polypeptide according to any one of aspects 1-39; and
   ii) the second nucleic acid comprises a nucleotide sequence encoding the second polypeptide according to any one of aspects 1-39.

Aspect 43. A composition comprising:
a) an expression vector comprising the nucleic acid of aspect 42(a); or
b) one or more expression vectors comprising the nucleic acids of aspects 42(b).

Aspect 44. A method of selectively modulating the activity of T cell specific for an epitope, the method comprising contacting the T cell with a T-cell modulatory multimeric polypeptide according to any one of aspects 1-39 or a homodimer according to aspect 40, wherein said contacting selectively modulates the activity of the epitope-specific T cell.

Aspect 45. A method of treating a patient having a cancer, the method comprising administering to the patient an effective amount of a pharmaceutical composition comprising T-cell modulatory multimeric polypeptide according to any one of aspects 1-39 or a homodimer according to aspect 40.

Aspect 46. The method of aspect 45, wherein the cancer is a carcinoma, sarcoma, melanoma, leukemia, or lymphoma.

Aspect 47. The method of aspect 45 or 46, wherein said administering is intramuscular, intravenous, peritumoral, or intratumoral.

Aspect 48. The method of any one of aspects 45-47, wherein the method comprises administering a SARS-CoV-2 vaccine to the patient prior to administering to the patient an effective amount of a pharmaceutical composition comprising T-cell modulatory multimeric polypeptide.

Aspect 49. The method of any one of aspects 45-48, comprising co-administering an immune checkpoint inhibitor to the patient, optionally wherein the immune checkpoint inhibitor is an antibody specific for PD-L1, PD-1, or CTLA4.

Aspect 50. A T-cell modulatory multimeric polypeptide (TMMP) comprising:
at least one heterodimer comprising:
a) a first polypeptide comprising:
   i) a peptide epitope having a length of from about 4 amino acids to about 25 amino acids; and
   ii) first major histocompatibility complex (MHC) polypeptide, wherein the first MHC polypeptide is a f2 microglobulin (β2M) polypeptide;
b) a second polypeptide comprising a second MHC polypeptide, wherein the second MHC polypeptide is an MHC class I heavy chain polypeptide;
c) an immunoglobulin (Ig) Fc polypeptide or a non-Ig scaffold, wherein the first or the second polypeptide comprises the Ig Fc polypeptide or the non-Ig scaffold;
d) a tumor-targeting polypeptide (TTP), wherein the first and/or the second polypeptide comprises the TTP;
wherein the TMMP does not include an immunomodulatory polypeptide, and
optionally wherein one or more of the individual components of the first and/or second polypeptide in each of the above TMMP combinations are linked together by one or more independently selected peptide linkers.

Aspect 51. A TMMP of aspect 50, wherein
a1) the first polypeptide comprises, in order from N-terminus to C-terminus:
   i) the peptide epitope; and
   ii) the β2M polypeptide; and
b1) the second polypeptide comprises, in order from N-terminus to C-terminus:
   i) the TTP;
   ii) the MHC class I heavy chain polypeptide; and
   iv) an Ig Fc polypeptide; or
a2) the first polypeptide comprises, in order from N-terminus to C-terminus:
   i) the peptide epitope; and
   ii) the β2M polypeptide; and
b2) the second polypeptide comprises, in order from N-terminus to C-terminus:
   i) the MHC class I heavy chain polypeptide;
   ii) an Ig Fc polypeptide; and
   iii) the TTP; or
a3) the first polypeptide comprises, in order from N-terminus to C-terminus:
   i) the peptide epitope; and
   ii) the β2M polypeptide; and
b3) the second polypeptide comprises, in order from N-terminus to C-terminus:
   i) the MHC class I heavy chain polypeptide;
   ii) the TTP; and
   iii) an Ig Fc polypeptide.;
   ii) the β2M polypeptide; and
optionally wherein one or more of the individual components of the first and/or second polypeptide in each of the above TMMP combinations are linked together by one or more independently selected peptide linkers.

Aspect 52. A TMMP of aspect 50 or aspect 51, wherein
the first polypeptide comprises, in order from N-terminus to C-terminus:
   i) the peptide epitope;
   ii) a peptide linker; and
   iii) the β2M polypeptide; and
the second polypeptide comprises, in order from N-terminus to C-terminus:
   i) the TTP;
   ii) a peptide linker;
   iii) the MHC class I heavy chain polypeptide;
   iv) a peptide linker; and
   v) the Ig Fc polypeptide,
optionally wherein the peptide linker between the TTP and the MHC class I heavy chain polypeptide comprises no more than about 12 amino acids, e.g., no more than 10 amino acids in length (e.g., a (GGGGS)₂ (SEQ ID NO:947) linker), no more than 8 amino acids in length, no more than 7 amino acids in length, no more than 6 amino acids in length, or no more than five amino acids in length, (e.g., a GGGGS (SEQ ID NO:873) linker).

Aspect 53. A TMMP of any one of aspects 50-52, wherein the second polypeptide comprises an Ig Fc polypeptide, and wherein the Ig Fc polypeptide is an IgG1 Fc polypeptide.

Aspect 54. A TMMP of aspect 53, wherein the IgG1 Fc comprises one or more amino acid substitutions that reduce or substantially eliminate antibody-dependent cell-mediated cytotoxicity (ADCC) and/or complement-dependent cytotoxicity (CDC).

Aspect 55. A TMMP of aspect 54, wherein IgG1 Fc polypeptide comprises L234A and L235A substitutions, wherein L234 and L235 correspond to amino acids 14 and 15 of the amino acid sequence depicted in FIG. 2A.

Aspect 56. A TMMP of aspect 52, wherein at least one of the one or more linkers comprises the amino acid sequence (GGGGS)n (SEQ ID NO:934), where n is an integer from 1 to 10.

Aspect 57. A TMMP of any one of aspects 50-56, wherein the TTP is a single-chain Fv (scFv) antibody or a nanobody.

Aspect 58. A TMMP of any one of aspects 50-57, wherein the TTP binds an antigen selected from mesothelin, TROP-2, Her2, CD19, WT1, MUC1, BCMA, PSMA, B7-H3, CEACAM, CD20, CD22, CD30, CD38, CD138, and a claudin polypeptide.

Aspect 59. A TMMP of any one of aspects 50-58, wherein the TTP binds a CD19 antigen.

Aspect 60. A TMMP of any one of aspects 50-59, wherein the peptide epitope has a length of from 8 amino acids to 14 amino acids, optionally from 9 amino acids to 12 amino acids.

Aspect 61. A TMMP of any one of aspects 50-60, wherein the peptide epitope comprises from 4 to 25 contiguous amino acids of a SARS-CoV-2 surface glycoprotein.

Aspect 62. A TMMP of aspect 61, wherein the SARS-CoV-2 peptide is a surface glycoprotein peptide depicted in FIG. 13J, optionally wherein the SARS-CoV-2 peptide is selected from the group consisting of STQDLFLPFF (SEQ ID NO:845), FCNDPFLGVYY (SEQ ID NO:846), SSANNCTFEY (SEQ ID NO:847), STECSNLLLQY (SEQ ID NO:848), YTNSFTRGVY (SEQ ID NO:849), CVADYSVLY (SEQ ID NO:850), LTDEMIAQY (SEQ ID NO:851), KIADYNYKL (SEQ ID NO:219), KLPDDFTGCV (SEQ ID NO:852), SFELLHAPATV (SEQ ID NO:853), LLFNKVTLA (SEQ ID NO:221), VLNDILSRL (SEQ ID NO:222), RLDKVEAEV (SEQ ID NO:223), RLQSLQTYV (SEQ ID NO:224), HLMSFPQSA (SEQ ID NO:225), RLNEVAKNL (SEQ ID NO:854), NLNESLIDL (SEQ ID NO:855), FIAGLIAIV (SEQ ID NO:226), VVFLHVTYV (SEQ ID NO:856), YLQPRTFLL (SEQ ID NO:218), SIIAYTMSL (SEQ ID NO:220), TLDSKTQSL (SEQ ID NO:534), KCYGVSPTK (SEQ ID NO:857), TVYDPLQPELDSFK (SEQ ID NO:333), GVYFASTEK (SEQ ID NO:289), GTHWFVTQR (SEQ ID NO:332), YYVGYLQPRTF (SEQ ID NO:392), NYNYLYRLF (SEQ ID NO:394), YFPLQSYGF (SEQ ID NO:395), VYDPLQPELDSF (SEQ ID NO:858), YEQYIKWPWYI (SEQ ID NO:859), KWPWYIWLGF (SEQ ID NO:860), GTITSGWTF (SEQ ID NO:861), QYIKWPWYI (SEQ ID NO:400), RFDNPVLPF (SEQ ID NO:389), EILDITPCSF (SEQ ID NO:862), FVFKNIDGY (SEQ ID NO:592), SVASQSIIAY (SEQ ID NO:863), WTAGAAAYY (SEQ ID NO:145), VASQSIIAY (SEQ ID NO:597), TPINLVRDL (SEQ ID NO:476), SPRRARSVA (SEQ ID NO:481), APHGVVFLHV (SEQ ID NO:864), LPQGFSAL (SEQ ID NO:477), QPYRVVVL (SEQ ID NO:479), MIAQYTSAL (SEQ ID NO:865), FPQSAPHGVVF (SEQ ID NO:600), SEPVLKGVKL (SEQ ID NO:484), TEKSNIIRGW (SEQ ID NO:711), TECSNLLLQY (SEQ ID NO:866), and REGVFVSNGTHW (SEQ ID NO:715).

Aspect 63. A TMMP any one of aspects 50-60, wherein the peptide epitope is a SARS-CoV-2 peptide epitope, and wherein the SARS-CoV-2 peptide has an amino acid sequence selected from YLQPRTFLL (SEQ ID NO:218), YLQPRTLFL (SEQ ID NO:887), YLQPRTLIL (SEQ ID NO:1201), and YLQPRTLVL (SEQ ID NO:1202), wherein the SARS-CoV-2 peptide has a length of 9 amino acids.

Aspect 64. A TMMP of any one of aspects 50-60, wherein the peptide epitope is a SARS-CoV-2 peptide epitope, and wherein the SARS-CoV-2 polypeptide is a non-structural polypeptide, optionally wherein the non-structural polypeptide is NSP13.

Aspect 65. A TMMP of aspect 64, wherein the peptide epitope is a SARS-CoV-2 peptide epitope, and wherein the SARS-CoV-2 peptide has the amino acid sequence VMPLSAPTL (SEQ ID NO:914) and has a length of 9 amino acids.

Aspect 66. A TMMP of any one of aspects 1-60, wherein the peptide epitope is an epitope of a pathogen other than a SARS-CoV-2 epitope, optionally where in the peptide epitope is a viral epitope of a virus selected from the group of Adenoviruses, Adeno-associated virus, Alphaviruses (Togaviruses), Eastern equine encephalitis virus, Eastern equine encephalomyelitis virus, Venezuelan equine encephalomyelitis vaccine strain TC-83, Western equine encephalomyelitis virus, Arenaviruses, Lymphocytic choriomeningitis virus (non-neurotropic strains), Tacaribe virus complex, Bunyaviruses, Bunyamwera virus, Rift Valley fever virus vaccine strain MP-12, Chikungunya virus, Calciviruses, Coronaviruses, Cowpox virus, Flaviviruses (Togaviruses)—Group B Arboviruses, Dengue virus serotypes 1, 2, 3, and 4, Yellow fever virus vaccine strain 17D, Hepatitis A, B, C, D, and E viruses, the Cytomegalovirus, Epstein Barr virus, Eastern Equine encephalitis virus, Herpes simplex types 1 and 2, Herpes zoster, Human herpesvirus types 6 and 7, hepatitis C virus (HVC), hepatitis B virus (HBV), Influenza viruses types A, B, and C, Papovaviruses, Newcastle disease virus, Measles virus, Mumps virus, Parainfluenza viruses types 1, 2, 3, and 4, polyomaviruses (JC virus, BK virus), Respiratory syncytial virus, Human parvovirus (B 19), Coxsackie viruses types A and B, Echoviruses, Polioviruses, Rhinoviruses, Alastrim (Variola minor virus), Smallpox (Variola major virus), Whitepox Reoviruses, Coltivirus, human Rotavirus, and Orbivirus (Colorado tick fever virus), Rabies virus, Vesicular stomatitis virus, Rubivirus (rubella), Semliki Forest virus, St. Louis encephalitis virus, Venezuelan equine encephalitis virus, Venezuelan equine encephalomyelitis virus, Arenaviruses (a.k.a. South American Hemorrhagic Fever virus), Flexal, Lymphocytic choriomeningitis virus (LCM) (neurotropic strains), Hantaviruses including Hantaan virus, Rift Valley fever virus, Japanese encephalitis virus, Yellow fever virus, Monkeypox virus, Human immunodeficiency virus (HIV) types 1 and 2, Human T cell lymphotropic virus (HTLV) types 1 and 2, Simian immunodeficiency virus (SIV), Vesicular stomatitis virus, Guanarito virus, Lassa fever virus, Junin virus, Machupo virus, Sabia, Crimean-Congo hemorrhagic fever virus, Ebola viruses, Marburg virus, Tick-borne encephalitis virus complex (flavi) including Central European tick-borne encephalitis, Far Eastern tick-borne encephalitis, Hanzalova, Hypr, Kumlinge, Kyasanur Forest disease, Omsk hemorrhagic fever, and Russian Spring Summer encephalitis viruses, Herpesvirus simiae (Herpes B or Monkey B virus), Cercopithecine herpesvirus 1 (Herpes B virus), Equine morbillivirus (Hendra and Hendra-like viruses), Nipah virus, Variola major virus (Smallpox virus), Variola minor virus (Alastrim), African swine fever virus, African horse sickness virus, Akabane virus, Avian influenza virus (highly pathogenic), Blue tongue virus, Camel pox virus, Classical swine fever virus, Cowdria ruminantium (heartwater), Foot and mouth disease virus, Goat pox virus, Japanese encephalitis virus, Lumpy skin disease virus, Malignant catarrhal fever virus, Menangle virus, Newcastle disease virus (VVND), Vesicular stomatitis virus (exotic), and Zika virus.

Aspect 67. A TMMP of aspect 66, where the viral antigen is a cytomegalovirus (CMV) polypeptide.

Aspect 68. A T-cell modulatory multimeric polypeptide of aspect 67, wherein the CMV polypeptide is a CMV pp65 polypeptide.

Aspect 69. A T-cell modulatory multimeric polypeptide of aspect 68, wherein the peptide has the amino acid sequence NLVPMVATV (SEQ ID NO:913) and has a length of 9 amino acids.

Aspect 70. A TMMP of any one of aspects 60-69, wherein the first polypeptide and the second polypeptide are covalently linked to one another via one or more disulfide bonds.

Aspect 71. A TMMP of aspect 70, wherein the β2M polypeptide and the MHC heavy chain polypeptide are joined by a disulfide bond that joins a Cys residue in the β2M polypeptide and a Cys residue in the MHC heavy chain polypeptide.

Aspect 72. A TMMP of aspect 71, wherein a Cys at amino acid residue 12 of the β2M polypeptide is disulfide bonded to a Cys at amino acid residue 236 of the MHC heavy chain polypeptide.

Aspect 73. A TMMP of aspect 70, wherein the first polypeptide chain comprises a linker between the peptide epitope and the β2M polypeptide, and wherein the disulfide bond links a Cys present in the linker with a Cys at position 84 of the MHC heavy chain polypeptide.

Aspect 74. A TMMP of any one of aspects 50-73, wherein the first and the second polypeptides are covalently linked to one another via at least 2 disulfide bonds.

Aspect 75. A TMMP of aspect 74, wherein:
a) a first disulfide bond is between: i) a Cys present in a linker between the peptide epitope and the β2M polypeptide; and ii) a Cys residue at position 84 in the MHC class I heavy chain polypeptide; and
b) a second disulfide bond is between: i) a Cys residue at position 12 in the β2M polypeptide; and ii) a Cys residue at position 236 in the MHC Class I heavy chain polypeptide.

Aspect 76. A TMMP of aspect 75, wherein the linker comprises the amino acid sequence GCGGS (SEQ ID NO:977) or amino acid sequence GCGGS(GGGGS)n (SEQ ID NO:978), where n is an integer from 1 to 10.

Aspect 77. A TMMP of any one of aspects 50-76, wherein the MHC class I heavy chain is an HLA-E allele heavy chain polypeptide.

Aspect 78. A TMMP of any one of aspects 52-77, wherein the peptide linker is no more than five amino acids in length and optionally has the amino acid sequence GGGGS (SEQ ID NO:873), or is no more than 10 amino acids in length and optionally has the amino acid sequence (GGGGS)₂ (SEQ ID NO:947).

Aspect 79. A TMMP of any one of aspects 52-78, wherein:
a) the first polypeptide comprises, in order from N-terminus to C-terminus:
   i) the SARS-CoV-2 peptide, wherein the SARS-CoV2 peptide comprises the amino acid sequence YLQPRTFLL (SEQ ID NO:218);
   ii) a peptide linker; and
   iii) the β2M polypeptide, wherein the β2M polypeptide comprises an amino acid sequence having at least 95% amino acid sequence identity to the β2M amino acid sequence depicted in FIG. 32G; and
b) the second polypeptide comprises, in order from N-terminus to C-terminus:
   i) the TTP, wherein the TTP is an anti-CD19 single chain Fv antibody;
   ii) a peptide linker;
   iii) the MHC class I heavy chain polypeptide, wherein the MHC class I heavy chain comprises an amino acid sequence having at least 95% amino acid sequence identity to the amino acid sequence depicted in FIG. 32D;
   iv) a peptide linker; and
   v) the Ig Fc polypeptide, wherein the Ig Fc polypeptide comprises an amino acid sequence having at least 95% amino acid sequence identity to the amino acid sequence depicted in FIG. 32E.

Aspect 80. A TMMP of aspect 79, wherein:
i) the β2M polypeptide comprises a Cys at position 12;
   ii) the TTP comprises an amino acid sequence having at least 95% amino acid sequence identity to the amino acid sequence depicted in FIG. 32C;
iii) the MHC class I heavy chain comprises a Cys at position 84 and a Cys at position 236; and
iv) the Ig Fc polypeptide comprises an Ala at position 14 and an Ala at position 15.

Aspect 81. A TMMP of aspect 80, wherein:
a) the first polypeptide comprises an amino acid sequence having at least 95% amino acid sequence identity to the amino acid sequence depicted in FIG. 32B; and
b) the second polypeptide comprises an amino acid sequence having at least 95% amino acid sequence identity to the amino acid sequence depicted in FIG. 33.

Aspect 82. A homodimer comprising two heterodimers according to any one of aspects 50-81, wherein the two heterodimers are covalently linked to one another by one or more disulfide bonds between Ig Fc polypeptides present in the first and second heterodimers.

Aspect 83. A pharmaceutical composition comprising a heterodimer of any one of aspects 50-81 or a homodimer of aspect 82.

Aspect 84. A composition comprising:
a) a nucleic acid comprising a nucleotide sequence encoding the first polypeptide and the second polypeptide according to any one of aspects 50-82; or
b) first and second nucleic acids, wherein:
   i) the first nucleic acid comprises a nucleotide sequence encoding the first polypeptide according to any one of aspects 50-82; and
ii) the second nucleic acid comprises a nucleotide sequence encoding the second polypeptide according to any one of aspects 1-39.

Aspect 85. A composition comprising:
a) an expression vector comprising the nucleic acid of aspect 84(a); or
b) one or more expression vectors comprising the nucleic acids of aspects 84(b).

Aspect 86. A method of selectively modulating the activity of T cell specific for an epitope, the method comprising contacting the T cell with a T-cell modulatory multimeric polypeptide according to any one of aspects 50-81 or a homodimer according to aspect 82, wherein said contacting selectively modulates the activity of the epitope-specific T cell.

Aspect 87. A method of treating a patient having a cancer, the method comprising administering to the patient an effective amount of a pharmaceutical composition comprising T-cell modulatory multimeric polypeptide according to any one of aspects 50-81 or a homodimer according to aspect 82.

Aspect 88. The method of aspect 87, wherein the cancer is a carcinoma, sarcoma, melanoma, leukemia, or lymphoma.

Aspect 89. The method of aspect 87 or 88, wherein said administering is intramuscular, intravenous, peritumoral, or intratumoral.

Aspect 90. The method of any one of aspects 87-89, wherein the method comprises administering a SARS-CoV-2 vaccine to the patient prior to administering to the patient an effective amount of a pharmaceutical composition comprising T-cell modulatory multimeric polypeptide.

Aspect 91. The method of any one of aspects 87-90, comprising co-administering an immune checkpoint inhibitor to the patient, optionally wherein the immune checkpoint inhibitor is an antibody specific for PD-L1, PD-1, or CTLA4.

Aspect 92. The method of aspect 91, wherein the checkpoint inhibitor is an antibody specific for PD-1, and the patient has a CPS score of 20 or less.

Aspect 93. The method of aspect 87, wherein cancer is a hematological cancer.

Aspect 94. The method of any one of aspects 87-93, wherein the cancer exhibits HLA loss.

### Aspects Set C

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. A T-cell modulatory multimeric polypeptide (TMMP) comprising at least one heterodimer comprising: a) a first polypeptide comprising from N-terminus to C-terminus: i) a SARS-CoV-2 peptide, wherein the SARS-CoV-2 peptide or a peptide of another pathogen has a length of from about 4 amino acids to about 25 amino acids, ii) an optional peptide linker, and iii) first major histocompatibility complex (MHC) polypeptide, wherein the first MHC polypeptide is a β2 microglobulin (β2M) polypeptide; and b) a second polypeptide comprising, from N-terminus to C-terminus: i) a tumor-targeting polypeptide (TTP), ii) a peptide linker, iii) a second MHC polypeptide, wherein the second MHC polypeptide is an MHC class I heavy chain polypeptide, iv) an optional linker, v) an immunoglobulin (Ig) Fc polypeptide, vi) an optional linker, and vii) at least one immunomodulatory polypeptide, wherein the peptide linker between the TTP and the MHC class I heavy chain polypeptide comprises no more than about 12 amino acids, e.g., no more than 10 amino acids in length (e.g., a (GGGGS)₂ (SEQ ID NO:947) linker), no more than 8 amino acids in length, no more than 7 amino acids in length, no more than 6 amino acids in length, or no more than 5 amino acids in length, (e.g., a GGGGS (SEQ ID NO:873) linker).

Aspect 2. The TMMP of aspect 1, wherein the peptide linker between the TTP and the MHC class I heavy chain polypeptide is 5 amino acids in length and has the amino acid sequence GGGGS or is 10 amino acids in length and has the amino acid sequence (GGGGS)₂ (SEQ ID NO:947).

Aspect 3. The TMMP of aspect 1, wherein the peptide linker between the TTP and the MHC class I heavy chain polypeptide is 5 amino acids in length and has the amino acid sequence GGGGS.

Aspect 4. The TMMP of any one of aspects 1-3, wherein the at least one immunomodulatory polypeptide is a variant IL-2 polypeptide.

Aspect 5. The TMMP of aspect 4, wherein the at least one variant IL-2 polypeptide binds to IL-2R and exhibits decreased or substantially no binding to IL-2Ra, and also exhibits decreased binding to IL-2Rβ such that the IL-2 variant polypeptide exhibits an overall reduced affinity for IL-2R, optionally wherein the at least one variant comprises substitutions at H16 and F42 selected from H16A, F42A; H16T, F42A; H16E, F42A; H16D, F42A; H16A, F42K; H16T, F42K; H16E, F42K; and H16D, F42K.

Aspect 6. The TMMP of aspect 5, wherein the at least one immunomodulatory polypeptide is a single copy of a variant IL-2 polypeptide.

Aspect 7. The TMMP of aspect 6, wherein the variant IL-2 polypeptide comprises substitutions at H16 and F42.

Aspect 8. The TMMP of aspect 7, wherein the variant IL-2 polypeptide comprises H16A and F42A substitutions.

Aspect 9. The TMMP of aspect 8, wherein the peptide linker between the TTP and the MHC class I heavy chain polypeptide has the amino acid sequence GGGGS (SEQ ID NO:873).

Aspect 10. The TMMP of aspect 9, wherein: the MHC class I heavy chain polypeptide is an HLA-A02 polypeptide and the SARS-CoV-2 peptide is YLQPRTFLL (SEQ ID NO:218); or the MHC class I heavy chain polypeptide is an HLA-E polypeptide and the SARS-CoV-2 peptide is VMPLSAPTL (SEQ ID NO:914) or YLQPRTFLL (SEQ ID NO:218).

Aspect 11. A homodimer comprising two heterodimers according to any one of aspects 1-10, wherein the two heterodimers are covalently linked to one another by one or more disulfide bonds between Ig Fc polypeptides present in the first and second heterodimers.

Aspect 12. A method of treating a patient having a cancer, the method comprising administering to the patient an effective amount of a pharmaceutical composition comprising an effective amount of a TMMP according to any one of aspects 1-10.

Aspect 13. A method of treating a patient having a cancer, the method comprising administering to the patient an effective amount of a pharmaceutical composition comprising an effective amount of a homodimer comprising two heterodimers according to any one of aspects 1-10, wherein the two heterodimers are covalently linked to one another by one or more disulfide bonds between Ig Fc polypeptides present in the first and second heterodimers.

Aspect 14. A T-cell modulatory multimeric polypeptide (TMMP) comprising at least one heterodimer comprising: a) a first polypeptide comprising: i) a SARS-CoV-2 peptide, wherein the SARS-CoV-2 peptide or a peptide of another pathogen has a length of from about 4 amino acids to about 25 amino acids, and ii) first major histocompatibility complex (MHC) polypeptide, wherein the first MHC polypeptide is a f2 microglobulin (β2M) polypeptide; and b) a second polypeptide comprising, from N-terminus to C-terminus: i) a tumor-targeting polypeptide (TTP), ii) a peptide linker, iii) a second MHC polypeptide, wherein the second MHC polypeptide is an MHC class I heavy chain polypeptide, iv) an optional linker, and v) an immunoglobulin (Ig) Fc polypeptide, wherein the peptide linker between the TTP and the MHC class I heavy chain polypeptide comprises no more than about 12 amino acids, e.g., no more than 10 amino acids in length (e.g., a (GGGGS)₂ (SEQ ID NO:947) linker), no more than 8 amino acids in length, no more than 7 amino acids in length, no more than 6 amino acids in length, or no more than five amino acids in length, (e.g., a GGGGS (SEQ ID NO:873) linker).

Aspect 15. The TMMP of aspect 14, wherein the peptide linker between the TTP and the MHC class I heavy chain polypeptide has the amino acid sequence GGGGS (SEQ ID NO:873) or (GGGGS)2 (SEQ ID NO:947).

Aspect 16. The TMMP of aspect 15, wherein the peptide linker between the TTP and the MHC class I heavy chain polypeptide has the amino acid sequence GGGGS (SEQ ID NO:873).

Aspect 17. The TMMP of any one of aspects 14-16, wherein: the MHC class I heavy chain polypeptide is an HLA-A02 polypeptide and the SARS-CoV-2 peptide is YLQPRTFLL; or the MHC class I heavy chain polypeptide is an HLA-E polypeptide and the SARS-CoV-2 peptide is VMPLSAPTL (SEQ ID NO:914) or YLQPRTFLL (SEQ ID NO:218).

Aspect 18. A homodimer comprising two heterodimers according to any one of aspects 14-17, wherein the two heterodimers are covalently linked to one another by one or more disulfide bonds between Ig Fc polypeptides present in the first and second heterodimers.

Aspect 19. A method of treating a patient having a cancer, the method comprising administering to the patient an effective amount of a pharmaceutical composition comprising an effective amount of a TMMP according to any one of aspects 14-17.

Aspect 20. A method of treating a patient having a cancer, the method comprising administering to the patient an effective amount of a pharmaceutical composition comprising an effective amount of a homodimer comprising two heterodimers according to any one of aspects 14-17, wherein the two heterodimers are covalently linked to one another by one or more disulfide bonds between Ig Fc polypeptides present in the first and second heterodimers.

Aspect 21. The method of any one of aspects 12, 13, 19 or 20, wherein the patient is further administered a checkpoint inhibitor that is an antibody specific for PD-1, and the patient has a CPS score of 20 or less.

Aspect 22. The method of any one of aspects 12, 13, 19, 20 or 21, wherein cancer is a hematological cancer.

Aspect 23. The method of any one of aspects 12, 13, 19 or 20-22, wherein the cancer exhibits HLA loss.

### Examples

The following example is put forth so as to provide those of ordinary skill in the art with a more complete disclosure and is not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

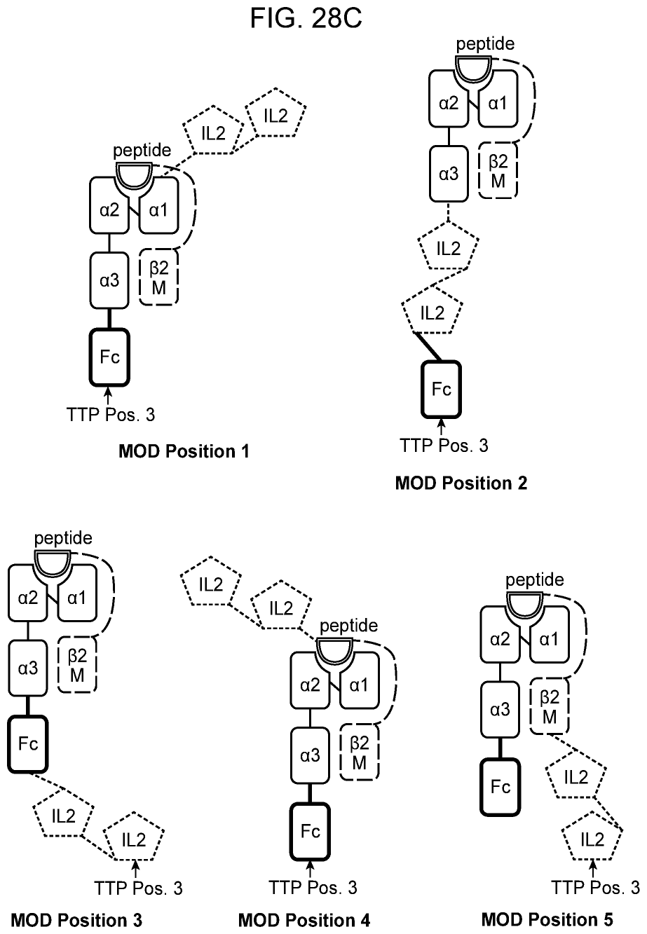

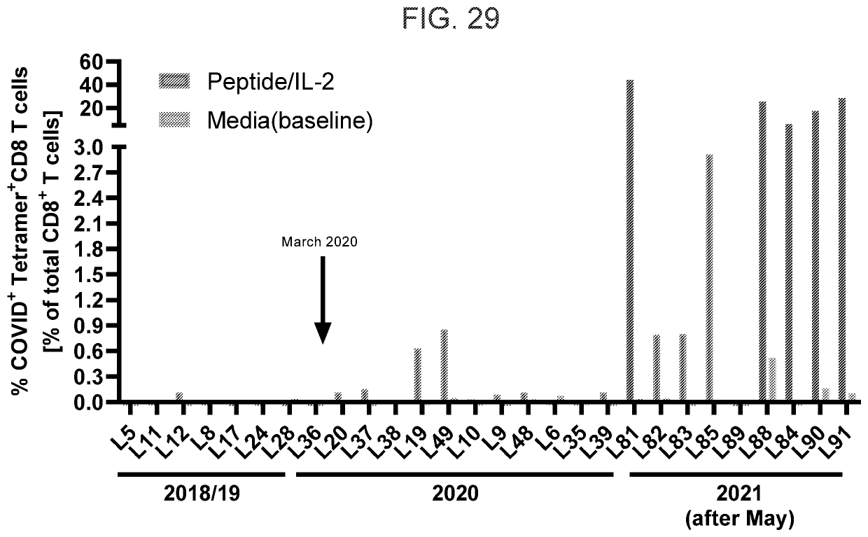

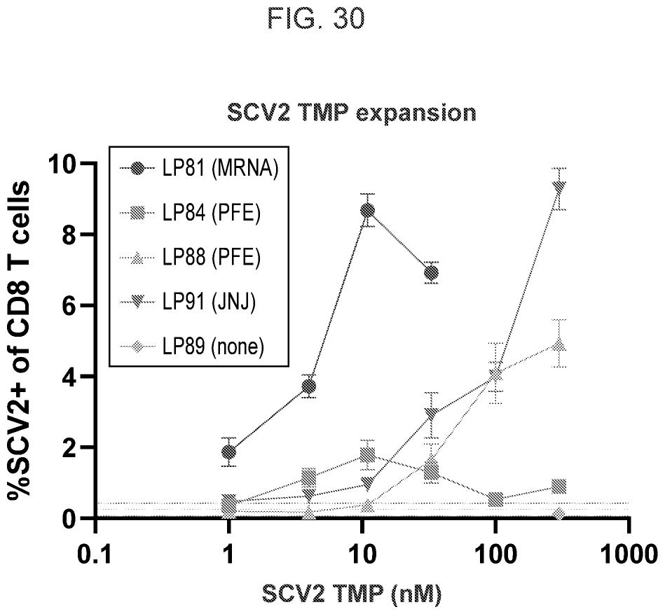

Peripheral blood mononuclear cells (PBMCs), obtained from human donors (Leukopaks, designated “L” in FIG. 29 and “LP” in FIG. 30) before the SARS CoV-2 pandemic and in the months following the start of the pandemic, were tested for reactivity of CD8⁺ T cells within the PBMCs to the SARS CoV-2 HLA-A*0201-restricted epitope YLQPRTFLL (SEQ ID NO:218). The data are shown in FIG. 29. “March 2020” indicates the start of lockdowns in the U.S. The availability to the public of the Moderna, the Pfizer/BioNTech, and the Johnson & Johnson (“Janssen”) vaccines is indicated as samples collected after May 2021. As shown in FIG. 29, reactivity to the SARS CoV-2 immunodominant HLA*A0201 peptide in healthy individuals was minimal before availability of the vaccines that compromise this peptide. Following availability of the various SARS-CoV-2 vaccines, reactivity to the SARS CoV-2 peptide increased to up to 60%, as expressed by the % of total CD8⁺ T cells that are reactive to the peptide.

PBMCs from previously identified donors who were vaccinated with a SARS CoV-2 vaccine were exposed in vitro with a dose response of a protein (designated “TMP” in FIG. 30) comprising a dimer of two T-cell modulatory polypeptides, each of which comprised HLA-A*0201 MHC class I polypeptides (β2M and heavy chain), a SARS CoV-2 peptide YLQPRTFLL (SEQ ID NO:218), two variant IL-2 (H16A, F42A) immunomodulatory polypeptides and a variant IgG1 Fc (L234A, L235A) polypeptide. Nine days after initial exposure, the percentage of CD8⁺ T cells that recognize the SARS CoV-2 peptide YLQPRTFLL (SEQ ID NO:218) in the context of HLA*0201 was tested via tetramer staining by flow cytometry. The results, depicted in FIG. 30, demonstrate that a T-cell modulatory protein comprising a SARS CoV-2 peptide can bind to and modulate SARS CoV-2-reactive CD8⁺ T cells.

### Example 2: SARS-CoV2-Specific T Cell Killing of CD19-Positive Cells

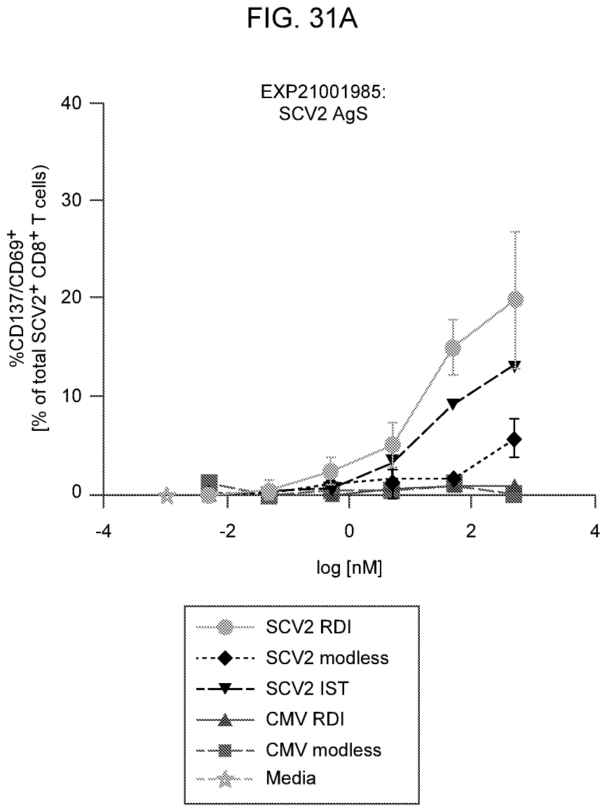

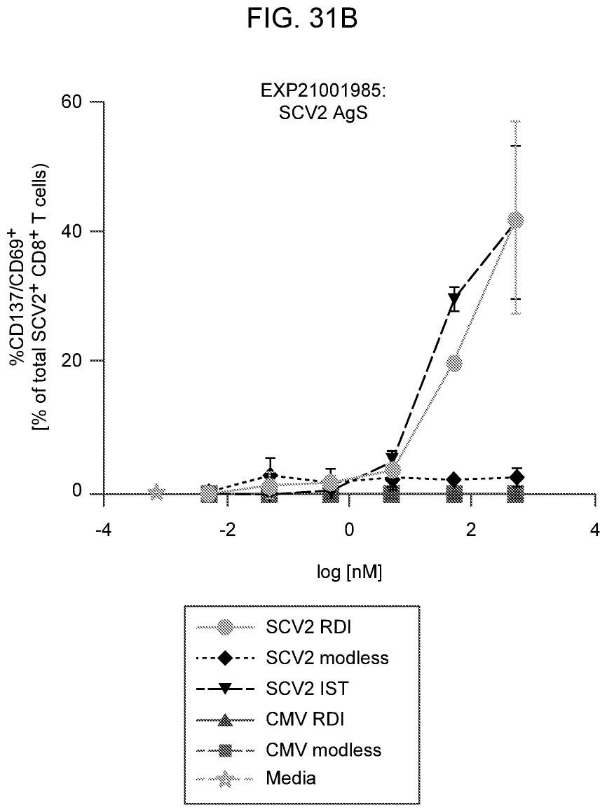

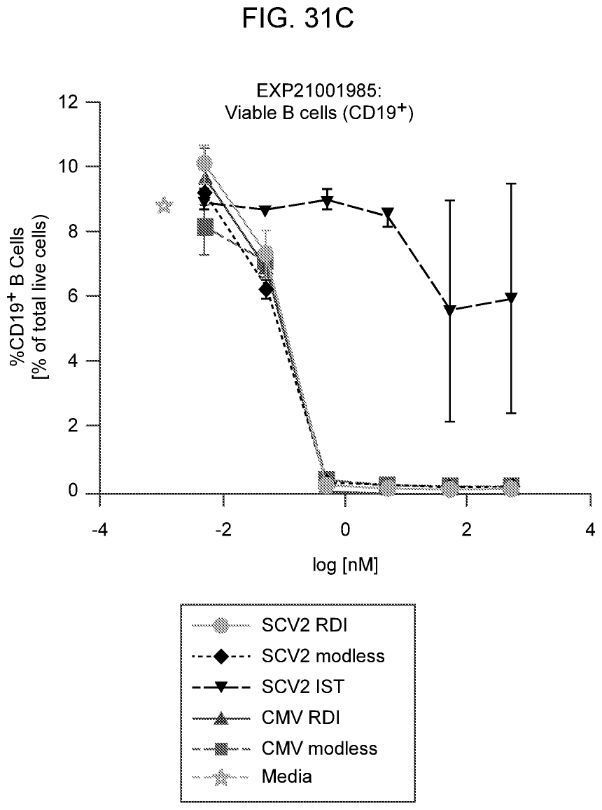

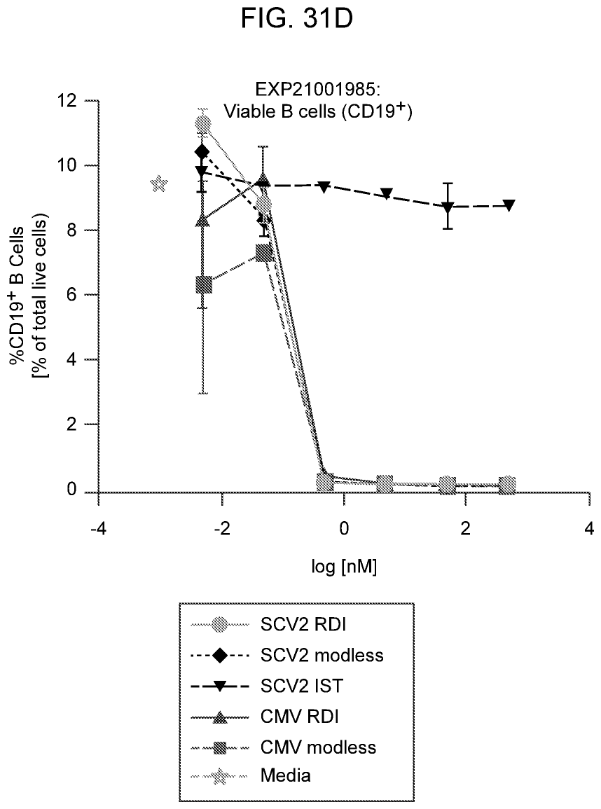

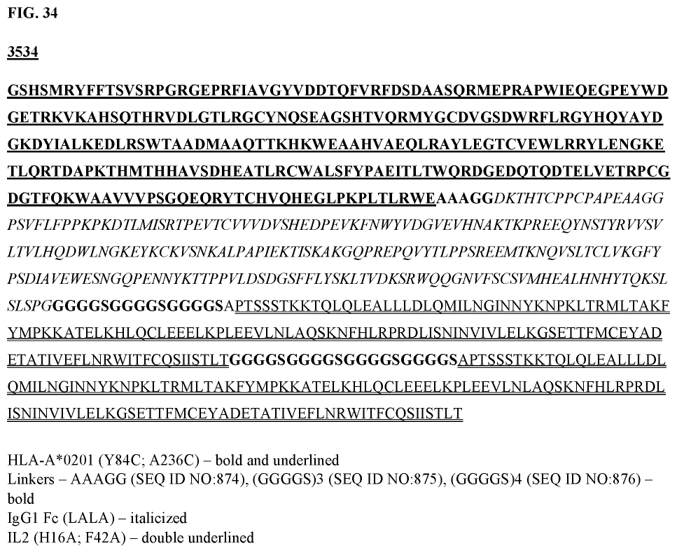

PBMCs from a SARS-CoV2-responsive and cytomegalovirus (CMV)-responsive donor were incubated with the following constructs:
i) a TMMP comprising a SARS-CoV2 peptide and an anti-CD19 scFv as the TTP, where the TMMP is referred to as “SCV2 RDI” in FIG. 31A-31D and has a first polypeptide designated “4700” in FIG. 32B and a second polypeptide designated “4518” in FIG. 32A. This TMMP may also be referred to as “4518-4700”;
ii) a control TMMP that is like SCV2 RDI, but without a MOD, where the control is designated “SCV2 modless” in FIG. 31A-31D and has a first polypeptide designated “4700” in FIG. 32B and a second polypeptide designated “4656” in FIG. 33. This TMMP may also be referred to as “4656-4700”;
iii) a multimeric polypeptide comprising a SARS-CoV2 peptide but does not include a TTP, where the multimeric polypeptide is designated “SCV2 IST” in FIG. 31A-31B, and has a first polypeptide designated “4700” in FIG. 32B, and a second polypeptide designated “3534” in FIG. 34. This multimeric polypeptide may also be referred to as “3534-4700”;
iv) a TMMP comprising a CMV peptide and an anti-CD19 scFv as the TTP, where the TMMP is referred to as “CMV RDI” in FIG. 31A-31D and has a first polypeptide designated “1717” in FIG. 35 and a second polypeptide designated “4518” in FIG. 32A. This TMMP may also be referred to as “4518-1717”;
v) a control TMMP that is like CMV RDI, but without a MOD, where the control is designated “CMV modless” in FIG. 31A-31D and have a first polypeptide designated “1717” in FIG. 35 and a second polypeptide designated “4656” in FIG. 33. This TMMP may also be referred to as “4656-1717”;
vi) a bispecific T-cell engager (BiTE) comprising anti-CD3 and anti-CD19; or
vii) a control BiTE comprising anti-CD3 and R-Gal (but no anti-CD19).

The PBMCs were from a donor that has at baseline has SCV2-reactive (0.05%) and CMV-reactive (0.5%) CD8 T cells in unstimulated PBMCs. The various constructs described above were added in a volume of 500 μL to PBMCs (2×10⁶ PBMCs in 500 μL R10 medium), to a total volume of 1 mL. The PBMCs were incubated at 37° C. with the various constructs for 24 to 36 hours. After the incubation period, the PBMCs were tested by flow cytometry for: 1) activation of SARS-CoV2 (SCV2)-specific T cells present in the PBMCs, by assessing co-expression of CD127 and CD69; and 2) their ability to kill CD19-positive target B cells.

Activation of SCV2-specific T cells was tested by analyzing cell surface expression of CD137 and CD69, and by reactivity of the CD137⁺ and CD69⁺ T cells with SCV2, specifically, reactivity with the SCV2 peptide YLQPRTFLL (SEQ ID NO:218). The data are shown in FIG. 31A and FIG. 31B, where FIG. 31A and FIG. 31B depict the results of two different experiments. As shown in FIG. 31A and FIG. 31B, the TMMP designated “SCV2 RDI” activated antigen-specific T cells in a dose-dependent manner.

Killing of the target B cells was measured as the total number of live B cells remaining in the well after 24 hours. The data are shown in FIG. 31C and FIG. 31D, where FIG. 31C and FIG. 31D depict the results of two different experiments. As shown in FIG. 31C and FIG. 31D, the TMMP designated “SCV2 RDI” induced killing of target CD19⁺ cells (native B cells) in a dose-dependent manner.

### Example 3: Dose-Dependent, Antigen-Specific, Expansion of T Cells

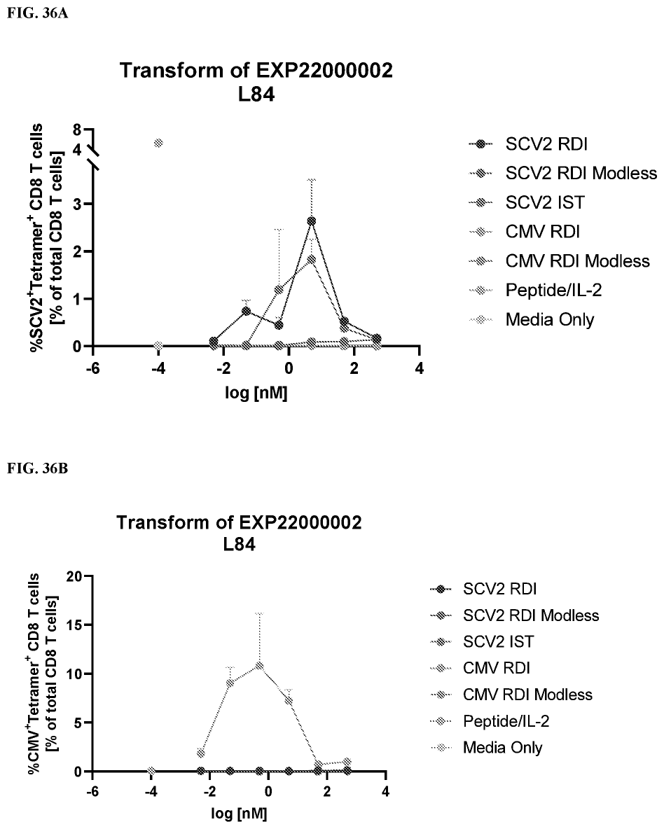

PBMCs from a donor (“L84”) reactive to both SARS-CoV-2 and CMV were incubated with various constructs. PBMCs were cultured in a 24-well plate at 2.5×10⁶ PBMCs/well in a total volume of 1 mL per well. Constructs were added to the wells at a final concentration of 500 nM, 50 nM, 5 nM, 0.5 nM, 0.05 nM, and 0.005 nM. The constructs were as follows:
Construct 4518-4700. This construct is referred to in FIG. 36 as “SCV2 RDI”;
Construct 4656-4700. This construct is referred to in FIG. 36 as “SCV2 RDI Modless”;
Construct 3534-4700. This construct is referred to in FIG. 36 as “SCV2 IST”;
Construct 4518-1717. This construct is referred to in FIG. 36 as “CMV RDI”;
Construct 4656-1717. This construct is referred to in FIG. 36 as “CMV RDI Modless.

Each of these constructs is described in Example 2; amino acid sequences of the first and second polypeptides of the constructs are provided in FIG. 32-35.

The PBMCs were incubated at 37° C. with the various constructs for 24 to 36 hours. After the incubation period, the PBMCs were tested by flow cytometry for SARS-CoV-2 (SCV)-specific and CMV-specific CD8⁺ T cell expansion. The data are shown in FIG. 36A-36B.

As shown in FIG. 36A, incubation of the L84 PBMCs with the 4518-4700 construct (“SCV2 RDI”; a TMMP comprising a SARS-CoV2 peptide and an anti-CD19 scFv as the TTP) or with the 3534-4700 construct (“SCV2 IST”; a multimeric polypeptide comprising a SARS-CoV2 peptide, and that is like SCV2 RDI but does not include a TTP) induced proliferation of SCV2-specific CD8⁺ T cells. The effect was antigen specific, as analogous constructs containing CMV as the peptide did not induce proliferation of SCV2-specific CD8⁺ T cells.

As shown in FIG. 36B, incubation of the L84 PBMCs with the 4518-1717 construct (“CMV-RDI”; a TMMP comprising a CMV peptide and an anti-CD19 scFv as the TTP) induced proliferation of CMV-specific CD8⁺ T cells. No other construct induced proliferation of CMV-specific CD8⁺ T cells, indicating that the effect is antigen specific.

### Example 4: TMMP-Mediated Killing of CD19⁺ Tumor Cells

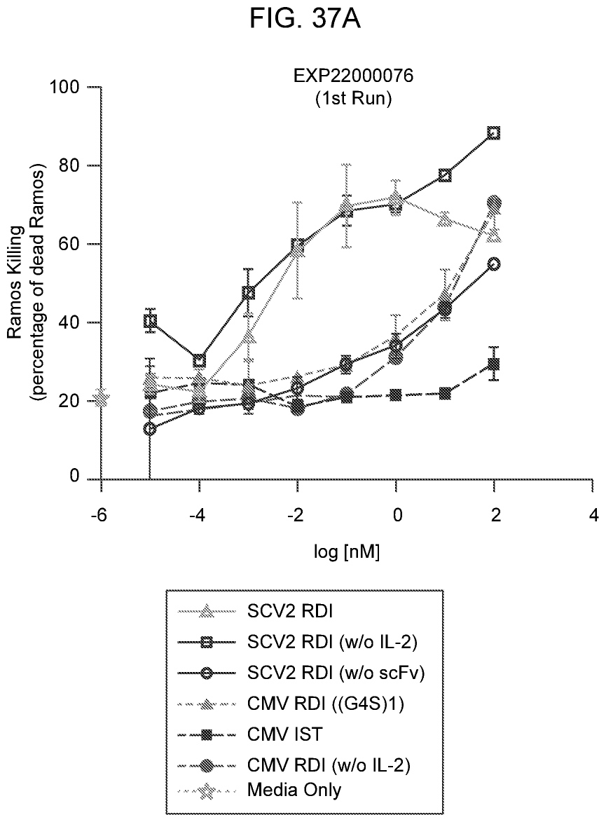

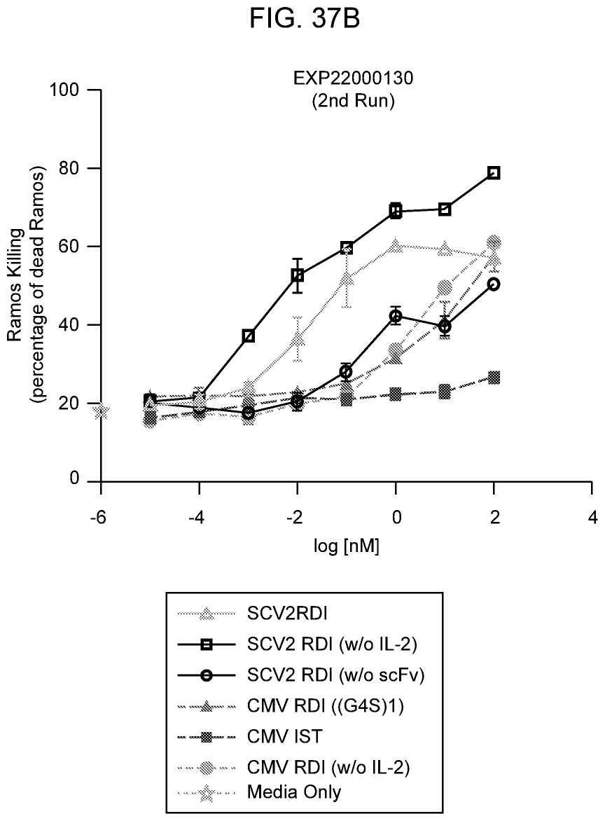

Constructs as described in Example 2 and Example 3 were incubated with (i) PBMCs (the same L84 PBMCs as in Examples 2 and 3) and (ii) Ramos cells (American Type Culture Collection (ATCC) No. CRL-1596), a CD19⁺ Burkitts lymphoma cell line. The effect of the various constructs on PBMC-mediated killing of the target Ramos cells was assessed. The results are shown in FIGS. 37A and 37B.

As shown in FIG. 37A, incubation of the L84 PBMCs with the 4518-4700 construct (“SCV2 RDI”; a TMMP comprising a SARS-CoV2 peptide and an anti-CD19 scFv as the TTP) induced killing of the Ramos cells in a concentration-dependent manner. The experiment was repeated; the results, shown in FIG. 37B, confirmed the data presented in FIG. 37A.

### Example 5

The effect of the linker between the scFv and the MHC class I heavy chain polypeptide, and the effect of the linker between the Ig Fc polypeptide and the MOD, on: i) the ability to induce killing of tumor cells; and ii) the ability to expand (induced proliferation of) anti-viral T cells, was assessed. Various constructs were made, which had different combinations of: i) the linker between the scFv and the MHC class I heavy chain polypeptide; and ii) the linker between the Ig Fc polypeptide and the MOD. The effect of these constructs on inducing tumor cell killing and on inducing proliferation of anti-viral T cells was determined using assays as described in Examples 2-4.

All constructs comprised: i) the SARS-CoV2 peptide YLQPRTFLL (SEQ ID NO:218); ii) an anti-CD19 scFv as the TTP, where the amino acid sequence of the anti-CD19 scFv is depicted in FIG. 38I; iii) an HLA-A*0201 (Y84C; A236C) heavy chain polypeptide, where the amino acid sequence of the HLA-A*0201 (Y84C; A236C) heavy chain polypeptide is depicted in FIG. 38J; and iv) an IgG Fc (LALA) polypeptide, where the amino acid sequence of the IgG Fc (LALA) polypeptide is depicted in FIG. 38K. The linker between the scFv and the HLA heavy chain polypeptide was varied, and was (GGGGS)n (SEQ ID NO:873), where n was 1, 2, 3, or 4. The linker between the Ig Fc polypeptide and the MOD was varied; and was (GGGGS)3 (SEQ ID NO:875) or (AP)4 (SEQ ID NO:963). Constructs comprised: i) no MOD; or ii) 1, 2, or 3 copies of IL-2(H16A; F42A) as the MOD, where the amino acid sequence of the IL-2(H16A; F42A) polypeptide is depicted in FIG. 38L.

Varying the Linker Between the scFv and the HLA Heavy Chain Polypeptide

The effect of the length of the linker between the scFv and the HLA heavy chain polypeptide on induction of target tumor cell killing induced by a TMMP was determined. The tumor cell target was RAMOS cells, and target tumor cell killing was assessed as described above. The linker between the scFv and the HLA heavy chain polypeptide was (GGGGS)n (SEQ ID NO:873), where n was 1, 2, 3, or 4. The constructs tested were as follows:

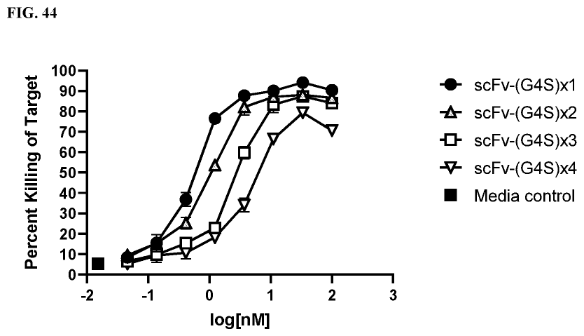

Construct 4518-4700. This construct has a first polypeptide designated “4700” in FIG. 32B and a second polypeptide designated “4518” in FIG. 32A. This construct is designated “scFv-(G4S)x1” in FIG. 44.

Construct 4519-4700. This construct has a first polypeptide designated “4700” in FIG. 32B and a second polypeptide designated “4519” in FIG. 42A. This construct is designated “scFv-(G4S)x2” in FIG. 44.

Construct 4520-4700. This construct has a first polypeptide designated “4700” in FIG. 32B and a second polypeptide designated “4520” in FIG. 42B. This construct is designated “scFv-(G4S)x3” in FIG. 44.

Construct 4337-4700. This construct has a first polypeptide designated “4700” in FIG. 32B and a second polypeptide designated “4337” in FIG. 42C. This construct is designated “scFv-(G4S)x4” in FIG. 44.

The features of these constructs are summarized in Table 6, below.

TABLE 6

| | 4519- | 4520- | 4337- | 4518- |
|---|---|---|---|---|
| | 4700 | 4700 | 4700 | 4700 |
| Linker | (G4S)2 | (G4S)3 | (G4S)4 | G4S |
| between | | | | |
| scFv and | | | | |
| HLA heavy | | | | |
| chain | | | | |
| Linker | (G4S)3 | (G4S)3 | (G4S)3 | (G4S)3 |
| between Fc | | | | |
| and MOD | | | | |
| MOD | 2 IL-2 | 2 IL-2 | 2 IL-2 | 2 IL-2 |
| 2^{nd} pptide | FIG. 42A | FIG. 42B | FIG. 42C | FIG. 32A |

The data are presented in FIG. 44.

The data show that TMMPs with shorter linkers between the scFv polypeptide and the HLA heavy chain polypeptide are more effective at inducing target tumor cell killing.

### Varying the Linker Between the Ig Fc Polypeptide and the Variant IL-2 Polypeptide and Varying the Number of MODs

The effect of variant IL-2 valency (i.e., the number of copies of variant IL-2 polypeptide per heavy chain polypeptide) and the effect of the linker between the Ig Fc polypeptide and the variant IL-2 polypeptide, was determined. The number of variant IL-2 polypeptide was none, 1, 2, or 3. The linker between the Ig Fc polypeptide and the variant IL-2 polypeptide was either (GGGGS)3 (SEQ ID NO:875) (“(G4S)3”) or (AP)4 (SEQ ID NO:963). The constructs tested were as follows:

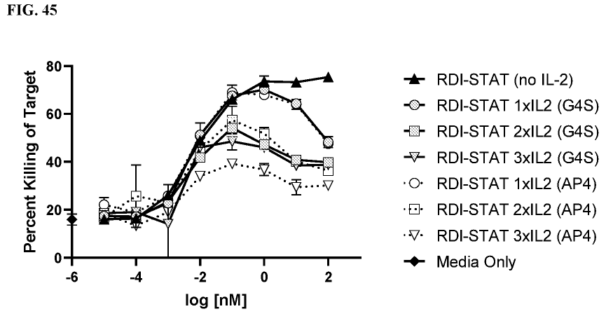

Construct 4656-4700. This construct is a “MOD-less” construct, and has a first polypeptide designated “4700” in FIG. 32B and a second polypeptide designated “4656” in FIG. 33. This construct is designated “RDI-STAT (no IL-2)” in FIG. 45.

Construct 4728-4700. This construct has a first polypeptide designated “4700” in FIG. 32B and a second polypeptide designated “4728” in FIG. 38A. This construct is designated “RDI-STAT 1×IL2 (G4S)” in FIG. 45.

Construct 4518-4700. This construct has a first polypeptide designated “4700” in FIG. 32B and a second polypeptide designated “4518” in FIG. 32A. This construct is designated “RDI-STAT 2×IL2 (G4S)” in FIG. 45.

Construct 4729-4700. This construct has a first polypeptide designated “4700” in FIG. 32B and a second polypeptide designated “4729” in FIG. 38D. This construct is designated “RDI-STAT 3×IL2 (G4S)” in FIG. 45.

Construct 4730-4700. This construct has a first polypeptide designated “4700” in FIG. 32B and a second polypeptide designated “4730” in FIG. 38B. This construct is designated “RDI-STAT 1×IL2 (AP4)” in FIG. 45.

Construct 4701-4700. This construct has a first polypeptide designated “4700” in FIG. 32B and a second polypeptide designated “4701” in FIG. 38C. This construct is designated “RDI-STAT 2×IL2 (AP4)” in FIG. 45.

Construct 4731-4700. This construct has a first polypeptide designated “4700” in FIG. 32B and a second polypeptide designated “4731” in FIG. 38E. This construct is designated “RDI-STAT 3×IL2 (AP4)” in FIG. 45.

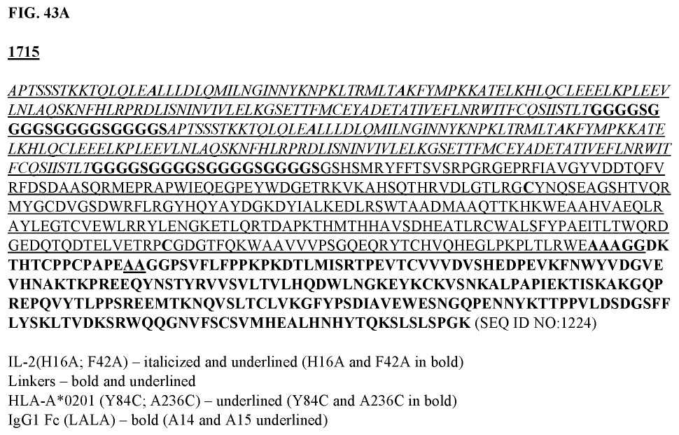

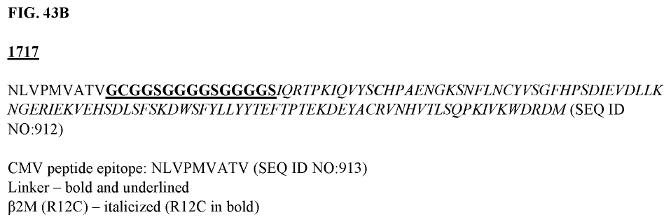

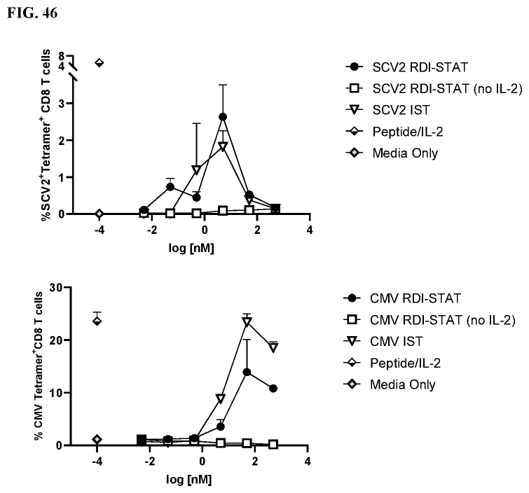

Construct 1715-4700. This construct has a first polypeptide designated “4700” in FIG. 32B and a second polypeptide designated “1715” in FIG. 43A. This construct is designated “SCV2 IST” in FIG. 46. This construct does not include a TTP.

Construct 1715-1717. This construct has a first polypeptide designated “1717” in FIG. 43A and a second polypeptide designated “1715” in FIG. 43A. This construct is designated “CMV IST” in FIG. 46. This construct includes a CMV peptide as the peptide epitope, and does not include a TTP.

Features of these constructs are summarized in Table 7, below.

TABLE 7

| | 4656- | 4728- | 4730- | 4518- | 4701- | 4729- | 4731- |
|---|---|---|---|---|---|---|---|
| | 4700 | 4700 | 4700 | 4700 | 4700 | 4700 | 4700 |
| Linker | G4S | G4S | G4S | G4S | G4S | G4S | G4S |
| between | | | | | | | |
| scFv and | | | | | | | |
| HLA heavy | | | | | | | |
| chain | | | | | | | |
| Linker | n/a | (G4S)3 | (AP)4 | (G4S)3 | (AP)4 | (G4S)3 | (AP)4 |
| between Fc | | | | | | | |
| and MOD | | | | | | | |
| MOD | (none) | 1 IL-2 | 1 IL-2 | 2 IL-2 | 2 IL-2 | 3 IL-2 | 3 IL-2 |
| 2^{nd} pptide | FIG. 33 | FIG. 38A | FIG. 38B | FIG. 32A | FIG. 38C | FIG. 38D | FIG. 38E |

The data are presented in FIG. 45 and FIG. 46.

For the data presented in FIG. 45, expanded SARS-CoV-2 (“SCV2”)-reactive T cells were used to kill target RAMOS cells. The effect of the copy number of IL-2, and the effect of either (GGGGS)3 (SEQ ID NO:875) or (AP4) (SEQ ID NO:963) as the linker between the Ig Fc and the variant IL-2 polypeptide, on TMMP-mediated killing was assessed. In the upper panel, “peptide/IL-2” refers to a composition comprising the SCV2 peptide (YLQPRTFLL; SEQ ID NO:218) and IL-2; this is a positive control for induction of T cell proliferation. In the lower panel, “peptide/IL-2” refers to a composition comprising the CMV peptide (NLVPMVATV; SEQ ID NO:913) and IL-2; this is a positive control for induction of T cell proliferation. As shown in FIG. 45, all TMMPs induced target tumor cell killing, and fewer copies of variant IL-2 resulted in more effective TMMP-induced target tumor cell killing.

For the data presented in FIG. 46, PBMCs from donors reactive to SCV2 were exposed to TMMPs comprising anti-CD19 scFv and SCV2 peptide (upper panel), while PBMCs from donors reactive to CMV were exposed to TMMPs comprising anti-CD19 scFv and CMV peptide (lower panel). As shown in FIG. 46, both anti-viral TMMPs caused a dose-dependent expansion of anti-viral CD8⁺ T cells.

While the present disclosure has been described with reference to the specific embodiments thereof, it should be understood by those skilled in the art that various changes may be made and equivalents may be substituted without departing from the true spirit and scope of the disclosure. In addition, many modifications may be made to adapt a particular situation, material, composition of matter, process, process step or steps, to the objective, spirit and scope. All such modifications are intended to be within the scope of the claims appended hereto.

What is claimed is:

1. A T-cell modulatory multimeric polypeptide (TMMP) comprising at least one heterodimer comprising:
a) a first polypeptide comprising, from N-terminus to C-terminus:
i) a SARS-CoV-2 peptide, wherein the SARS-CoV-2 peptide has a length of from about 4 amino acids to about 25 amino acids,
ii) an optional peptide linker, and
iii) first major histocompatibility complex (MHC) polypeptide, wherein the first MHC polypeptide is a f2 microglobulin (β2M) polypeptide; and
b) a second polypeptide comprising, from N-terminus to C-terminus:
i) a tumor-targeting polypeptide (TTP),
ii) a peptide linker,
iii) a second MHC polypeptide, wherein the second MHC polypeptide is an MHC class I heavy chain polypeptide,
iv) an optional peptide linker,
v) an immunoglobulin (Ig) Fc polypeptide,
vi) an optional peptide linker, and
vii) at least one immunomodulatory polypeptide,
wherein the peptide linker between the TTP and the MHC class I heavy chain polypeptide comprises no more than about 12 amino acids, optionally wherein the peptide linker is no more than 10 amino acids in length (e.g., a (GGGGS)₂ linker), no more than 8 amino acids in length, no more than 7 amino acids in length, no more than 6 amino acids in length, or no more than 5 amino acids in length, (e.g., a GGGGS linker).

2. The TMMP of claim 1, wherein the peptide linker between the TTP and the MHC class I heavy chain polypeptide is 5 amino acids in length and has the amino acid sequence GGGGS or is 10 amino acids in length and has the amino acid sequence (GGGGS)₂.

3. The TMMP of claim 1, wherein the peptide linker between the TTP and the MHC class I heavy chain polypeptide is 5 amino acids in length and has the amino acid sequence GGGGS.

4. The TMMP of claim 1, wherein the at least one immunomodulatory polypeptide is a variant IL-2 polypeptide.

5. The TMMP of claim 4, wherein the at least one variant IL-2 polypeptide binds to IL-2R and exhibits decreased or substantially no binding to IL-2Ra, and also exhibits decreased binding to IL-2Rβ such that the IL-2 variant polypeptide exhibits an overall reduced affinity for IL-2R, optionally wherein the at least one variant comprises substitutions at H16 and F42 selected from H16A, F42A; H16T, F42A; H16E, F42A; H16D, F42A; H16A, F42K; H16T, F42K; H16E, F42K; and H16D, F42K.

6. The TMMP of claim 5, wherein the at least one immunomodulatory polypeptide is a single copy of a variant IL-2 polypeptide.

7. The TMMP of claim 6, wherein the variant IL-2 polypeptide comprises substitutions at H16 and F42.

8. The TMMP of claim 7, wherein the variant IL-2 polypeptide comprises H16A and F42A substitutions.

9. The TMMP of claim 8, wherein the peptide linker between the TTP and the MHC class I heavy chain polypeptide has the amino acid sequence GGGGS.

10. The TMMP of claim 9, wherein
the MHC class I heavy chain polypeptide is an HLA-A02 polypeptide and the SARS-CoV-2 peptide is YLQPRTFLL, or
the MHC class I heavy chain polypeptide is an HLA-E polypeptide and the SARS-CoV-2 peptide is VMPLSAPTL or YLQPRTFLL.

11. A homodimer comprising two heterodimers according to any one of claims 1-10, wherein the two heterodimers are covalently linked to one another by one or more disulfide bonds between Ig Fc polypeptides present in the first and second heterodimers.

12. A method of treating a patient having a cancer, the method comprising administering to the patient an effective amount of a pharmaceutical composition comprising an effective amount of a TMMP according to any one of claims 1-10.

13. A method of treating a patient having a cancer, the method comprising administering to the patient an effective amount of a pharmaceutical composition comprising an effective amount of a homodimer comprising two heterodimers according to any one of claims 1-10, wherein the two heterodimers are covalently linked to one another by one or more disulfide bonds between Ig Fc polypeptides present in the first and second heterodimers.

14. A T-cell modulatory multimeric polypeptide (TMMP) comprising at least one heterodimer comprising:
a) a first polypeptide comprising
i) a SARS-CoV-2 peptide, wherein the SARS-CoV-2 peptide has a length of from about 4 amino acids to about 25 amino acids,
ii) an optional peptide linker, and
iii) first major histocompatibility complex (MHC) polypeptide, wherein the first MHC polypeptide is a f2 microglobulin (β2M) polypeptide; and
b) a second polypeptide comprising, from N-terminus to C-terminus
i) a tumor-targeting polypeptide (TTP),
ii) a peptide linker,
iii) a second MHC polypeptide, wherein the second MHC polypeptide is an MHC class I heavy chain polypeptide,
iv) an optional peptide linker, and
v) an immunoglobulin (Ig) Fc polypeptide,
wherein the peptide linker between the TTP and the MHC class I heavy chain polypeptide comprises no more than about 12 amino acids, optionally wherein the peptide linker is no more than 10 amino acids in length (e.g., a (GGGGS)₂ linker), no more than 8 amino acids in length, no more than 7 amino acids in length, no more than 6 amino acids in length, or no more than five amino acids in length, (e.g., a GGGGS linker).

15. The TMMP of claim 14, wherein the peptide linker between the TTP and the MHC class I heavy chain polypeptide has the amino acid sequence GGGGS or (GGGGS)2.

16. The TMMP of claim 15, wherein the peptide linker between the TTP and the MHC class I heavy chain polypeptide has the amino acid sequence GGGGS.

17. The TMMP of claim 16, wherein:
the MHC class I heavy chain polypeptide is an HLA-A02 polypeptide and the SARS-CoV-2 peptide is YLQPRTFLL, or
the MHC class I heavy chain polypeptide is an HLA-E polypeptide and the SARS-CoV-2 peptide is VMPLSAPTL or YLQPRTFLL.

18. A homodimer comprising two heterodimers according to any one of claims 14-17, wherein the two heterodimers are covalently linked to one another by one or more disulfide bonds between Ig Fc polypeptides present in the first and second heterodimers.

19. A method of treating a patient having a cancer, the method comprising administering to the patient an effective amount of a pharmaceutical composition comprising an effective amount of a TMMP according to any one of claims 14-17.

20. A method of treating a patient having a cancer, the method comprising administering to the patient an effective amount of a pharmaceutical composition comprising an effective amount of a homodimer comprising two heterodimers according to any one of claims 14-17, wherein the two heterodimers are covalently linked to one another by one or more disulfide bonds between Ig Fc polypeptides present in the first and second heterodimers.


SEQUENCE LISTING (1245 sequences)

SEQ ID NO: 1 - protein, 99 aa, Homo sapiens [sequence omitted]

SEQ ID NO: 2 - protein, 99 aa, synthetic construct
IQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 3 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 227 aa, synthetic construct
DKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 325 aa, Homo sapiens
STKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 10 - protein, 246 aa, Homo sapiens
HKPSNTKVDKRVELKTPLGDTTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 383 aa, Homo sapiens
PTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPRSTTFWAWSVLRVPAPPSPQPATYTCVVSHEDSRTLLNASRSLEVSYVTDHGPMK

SEQ ID NO: 12 - protein, 276 aa, Homo sapiens
VTSTLTIKZSDWLGESMFTCRVDHRGLTFQQNASSMCVPDQDTAIRVFAIPPSFASIFLTKSTKLTCLVTDLTTYBSVTISWTREENGAVKTHTNISESHPNATFSAVGEASICEDBDWSGERFTCTVTHTDLPSPLKQTISRPKGVALHRPBVYLLPPARZZLNLRESATITCLVTGFSPADVFVEWMQRGEPLSPQKYVTSAPMPEPQAPGRYFAHSILTVSEEEWNTGGTYTCVVAHEALPNRVTERTVDKSTGKPTLYNVSLVMSDTAGTCY

SEQ ID NO: 13 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 14 - protein, 222 aa, Homo sapiens
ADPCDSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 15 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 16 - protein, 275 aa, Homo sapiens [sequence omitted]

SEQ ID NO: 17 - protein, 275 aa, synthetic construct
GSHSMRYFFTSVSRPGRGEPRFIAVGYVDDTQFVRFDSDAASQRMEPRAPWIEQEGPEYWDGETRKVKAHSQTHRVDLGTLRGAYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWE

SEQ ID NO: 18 - protein, 275 aa, synthetic construct
GSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWE

SEQ ID NO: 19 - protein, 275 aa, synthetic construct
GSHSMRYFFTSVSRPGRGEPRFIAVGYVDDTQFVRFDSDAASQRMEPRAPWIEQEGPEYWDGETRKVKAHSQTHRVDLGTLRGAYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWE

SEQ ID NO: 20 - protein, 275 aa, synthetic construct
GSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMCAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWE

SEQ ID NO: 21 - protein, 275 aa, Homo sapiens
GSHSMRYFYTSVSRPGRGEPRFIAVGYVDDTQFVRFDSDAASQRMEPRAPWIEQEGPEYWDQETRNVKAQSQTDRVDLGTLRGYYNQSEDGSHTIQIMYGCDVGPDGRFLRGYRQDAYDGKDYIALNEDLRSWTAADMAAQITKRKWEAAHAAEQQRAYLEGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWE

SEQ ID NO: 22 - protein, 275 aa, synthetic construct
GSHSMRYFYTSVSRPGRGEPRFIAVGYVDDTQFVRFDSDAASQRMEPRAPWIEQEGPEYWDQETRNVKAQSQTDRVDLGTLRGAYNQSEDGSHTIQIMYGCDVGPDGRFLRGYRQDAYDGKDYIALNEDLRSWTAADMAAQITKRKWEAAHAAEQQRAYLEGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWE

SEQ ID NO: 23 - protein, 275 aa, synthetic construct
GSHSMRYFYTSVSRPGRGEPRFIAVGYVDDTQFVRFDSDAASQRMEPRAPWIEQEGPEYWDQETRNVKAQSQTDRVDLGTLRGCYNQSEDGSHTIQIMYGCDVGPDGRFLRGYRQDAYDGKDYIALNEDLRSWTAADMAAQITKRKWEAAHAAEQQRAYLEGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWE

SEQ ID NO: 24 - protein, 275 aa, synthetic construct
GSHSMRYFYTSVSRPGRGEPRFIAVGYVDDTQFVRFDSDAASQRMEPRAPWIEQEGPEYWDQETRNVKAQSQTDRVDLGTLRGAYNQSEDGSHTIQIMYGCDVGPDGRFLRGYRQDAYDGKDYIALNEDLRSWTAADMAAQITKRKWEAAHAAEQQRAYLEGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWE

SEQ ID NO: 25 - protein, 275 aa, synthetic construct
GSHSMRYFYTSVSRPGRGEPRFIAVGYVDDTQFVRFDSDAASQRMEPRAPWIEQEGPEYWDQETRNVKAQSQTDRVDLGTLRGCYNQSEDGSHTIQIMYGCDVGPDGRFLRGYRQDAYDGKDYIALNEDLRSWTAADMCAQITKRKWEAAHAAEQQRAYLEGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWE

SEQ ID NO: 26 - protein, 275 aa, Homo sapiens
GSHSMRYFSTSVSRPGRGEPRFIAVGYVDDTQFVRFDSDAASQRMEPRAPWIEQEGPEYWDEETGKVKAHSQTDRENLRIALRYYNQSEAGSHTLQMMFGCDVGSDGRFLRGYHQYAYDGKDYIALKEDLRSWTAADMAAQITKRKWEAAHVAEQQRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWE

SEQ ID NO: 27 - protein, 275 aa, synthetic construct
GSHSMRYFSTSVSRPGRGEPRFIAVGYVDDTQFVRFDSDAASQRMEPRAPWIEQEGPEYWDEETGKVKAHSQTDRENLRIALRAYNQSEAGSHTLQMMFGCDVGSDGRFLRGYHQYAYDGKDYIALKEDLRSWTAADMAAQITKRKWEAAHVAEQQRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWE

SEQ ID NO: 28 - protein, 275 aa, synthetic construct
GSHSMRYFSTSVSRPGRGEPRFIAVGYVDDTQFVRFDSDAASQRMEPRAPWIEQEGPEYWDEETGKVKAHSQTDRENLRIALRCYNQSEAGSHTLQMMFGCDVGSDGRFLRGYHQYAYDGKDYIALKEDLRSWTAADMAAQITKRKWEAAHVAEQQRAYLEGTCVDGLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWE

SEQ ID NO: 29 - protein, 275 aa, synthetic construct
GSHSMRYFSTSVSRPGRGEPRFIAVGYVDDTQFVRFDSDAASQRMEPRAPWIEQEGPEYWDEETGKVKAHSQTDRENLRIALRAYNQSEAGSHTLQMMFGCDVGSDGRFLRGYHQYAYDGKDYIALKEDLRSWTAADMAAQITKRKWEAAHVAEQQRAYLEGTCVDGLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWE

SEQ ID NO: 30 - protein, 275 aa, synthetic construct
GSHSMRYFSTSVSRPGRGEPRFIAVGYVDDTQFVRFDSDAASQRMEPRAPWIEQEGPEYWDEETGKVKAHSQTDRENLRIALRCYNQSEAGSHTLQMMFGCDVGSDGRFLRGYHQYAYDGKDYIALKEDLRSWTAADMCAQITKRKWEAAHVAEQQRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWE

SEQ ID NO: 31 - protein, 275 aa, Homo sapiens
GSHSMRYFTTSVSRPGRGEPRFIAVGYVDDTQFVRFDSDAASQRMEPRAPWIEQEGPEYWDRNTRNVKAHSQIDRVDLGTLRGYYNQSEAGSHTIQMMYGCDVGSDGRFLRGYQQDAYDGKDYIALNEDLRSWTAADMAAQITQRKWEAARVAEQLRAYLEGTCVEWLRRYLENGKETLQRTDPPKTHMTHHAVSDHEATLRCWALSFYPAEITLTWQRDGEDQTQDTELVETRPAGDGTFQKWASVVVPSGQEQRYTCHVQHEGLPKPLTLRWE

SEQ ID NO: 32 - protein, 275 aa, synthetic construct
GSHSMRYFTTSVSRPGRGEPRFIAVGYVDDTQFVRFDSDAASQRMEPRAPWIEQEGPEYWDRNTRNVKAHSQIDRVDLGTLRGAYNQSEAGSHTIQMMYGCDVGSDGRFLRGYQQDAYDGKDYIALNEDLRSWTAADMAAQITQRKWEAARVAEQLRAYLEGTCVEWLRRYLENGKETLQRTDPPKTHMTHHAVSDHEATLRCWALSFYPAEITLTWQRDGEDQTQDTELVETRPAGDGTFQKWASVVVPSGQEQRYTCHVQHEGLPKPLTLRWE

SEQ ID NO: 33 - protein, 275 aa, synthetic construct
GSHSMRYFTTSVSRPGRGEPRFIAVGYVDDTQFVRFDSDAASQRMEPRAPWIEQEGPEYWDRNTRNVKAHSQIDRVDLGTLRGCYNQSEAGSHTIQMMYGCDVGSDGRFLRGYQQDAYDGKDYIALNEDLRSWTAADMAAQITQRKWEAARVAEQLRAYLEGTCVEWLRRYLENGKETLQRTDPPKTHMTHHAVSDHEATLRCWALSFYPAEITLTWQRDGEDQTQDTELVETRPCGDGTFQKWASVVVPSGQEQRYTCHVQHEGLPKPLTLRWE

SEQ ID NO: 34 - protein, 275 aa, synthetic construct
GSHSMRYFTTSVSRPGRGEPRFIAVGYVDDTQFVRFDSDAASQRMEPRAPWIEQEGPEYWDRNTRNVKAHSQIDRVDLGTLRGAYNQSEAGSHTIQMMYGCDVGSDGRFLRGYQQDAYDGKDYIALNEDLRSWTAADMAAQITQRKWEAARVAEQLRAYLEGTCVEWLRRYLENGKETLQRTDPPKTHMTHHAVSDHEATLRCWALSFYPAEITLTWQRDGEDQTQDTELVETRPCGDGTFQKWASVVVPSGQEQRYTCHVQHEGLPKPLTLRWE

SEQ ID NO: 35 - protein, 275 aa, synthetic construct
GSHSMRYFTTSVSRPGRGEPRFIAVGYVDDTQFVRFDSDAASQRMEPRAPWIEQEGPEYWDRNTRNVKAHSQIDRVDLGTLRGCYNQSEAGSHTIQMMYGCDVGSDGRFLRGYQQDAYDGKDYIALNEDLRSWTAADMCAQITQRKWEAARVAEQLRAYLEGTCVEWLRRYLENGKETLQRTDPPKTHMTHHAVSDHEATLRCWALSFYPAEITLTWQRDGEDQTQDTELVETRPAGDGTFQKWASVVVPSGQEQRYTCHVQHEGLPKPLTLRWE

SEQ ID NO: 36 - protein, 276 aa, Homo sapiens
GSHSMRYFFTSVSRPGRGEPRFIAVGYVDDTQFVRFDSDAASQKMEPRAPWIEQEGPEYWDQETRNMKAHSQTDRANLGTLRGYYNQSEDGSHTIQIMYGCDVGPDGRFLRGYRQDAYDGKDYIALNEDLRSWTAADMAAQITKRKWEAVHAAEQRRVYLEGRCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEL

SEQ ID NO: 37 - protein, 276 aa, Homo sapiens [sequence omitted]

SEQ ID NO: 38 - protein, 276 aa, Homo sapiens
GSHSMRYFFTSVSRPGRGEPRFIAVGYVDDTQFVRFDSDAASQRMEPRAPWIEQEGPEYWDQETRNVKAQSQTDRVDLGTLRGYYNQSEAGSHTIQIMYGCDVGSDGRFLRGYRQDAYDGKDYIALNEDLRSWTAADMAAQITKRKWEAAHEAEQLRAYLDGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEL

SEQ ID NO: 39 - protein, 276 aa, Homo sapiens
GSHSMRYFYTSVSRPGRGEPRFIAVGYVDDTQFVRFDSDAASQRMEPRAPWIEQEGPEYWDQETRNVKAQSQTDRVDLGTLRGYYNQSEDGSHTIQIMYGCDVGPDGRFLRGYRQDAYDGKDYIALNEDLRSWTAADMAAQITKRKWEAAHAAEQQRAYLEGRCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEL

SEQ ID NO: 40 - protein, 276 aa, Homo sapiens
GSHSMRYFSTSVSRPGRGEPRFIAVGYVDDTQFVRFDSDAASQRMEPRAPWIEQEGPEYWDEETGKVKAHSQTDRENLRIALRYYNQSEAGSHTLQMMFGCDVGSDGRFLRGYHQYAYDGKDYIALKEDLRSWTAADMAAQITQRKWEAARVAEQLRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEP

SEQ ID NO: 41 - protein, 276 aa, Homo sapiens
GSHSMRYFSTSVSRPGRGEPRFIAVGYVDDTQFVRFDSDAASQRMEPRAPWIEQEGPEYWDEETGKVKAHSQTDRENLRIALRYYNQSEAGSHTLQMMFGCDVGSDGRFLRGYHQYAYDGKDYIALKEDLRSWTAADMAAQITKRKWEAAHVAEQQRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEP

SEQ ID NO: 42 - protein, 276 aa, Homo sapiens
GSHSMRYFSTSVSRPGRGEPRFIAVGYVDDTQFVRFDSDAASQRMEPRAPWIEQEGPEYWDEETGKVKAQSQTDRENLRIALRYYNQSEAGSHTLQMMFGCDVGSDGRFLRGYHQYAYDGKDYIALKEDLRSWTAADMAAQITKRKWEAAHVAEQQRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEP

SEQ ID NO: 43 - protein, 276 aa, Homo sapiens
GSHSMRYFTTSVSRPGRGEPRFIAVGYVDDTQFVRFDSDAASQRMEPRAPWIEQEGPEYWDRNTRNVKAHSQIDRVDLGTLRGYYNQSEAGSHTIQMMYGCDVGSDGRFLRGYQQDAYDGKDYIALNEDLRSWTAADMAAQITQRKWEAARVAEQLRAYLEGTCVEWLRRYLENGKETLQRTDPPKTHMTHHAVSDHEATLRCWALSFYPAEITLTWQRDGEDQTQDTELVETRPAGDGTFQKWASVVVPSGQEQRYTCHVQHEGLPKPLTLRWEP

SEQ ID NO: 44 - protein, 276 aa, Homo sapiens
GSHSMRYFYTSVSRPGRGEPRFIAVGYVDDTQFVRFDSDAASQRMEPRAPWIEQEGPEYWDRNTRKVKAQSQTDRVDLGTLRGYYNQSEDGSHTIQRMYGCDVGPDGRFLRGYQQDAYDGKDYIALNEDLRSWTAADMAAQITQRKWETAHEAEQWRAYLEGTCVEWLRRYLENGKETLQRTDAPKTHMTHHAVSDHEATLRCWALSFYPAEITLTWQRDGEDQTQDTELVETRPAGDGTFQKWASVVVPSGQEQRYTCHVQHEGLPKPLTLRWEP

SEQ ID NO: 45 - protein, 276 aa, synthetic construct
  REGION 1..276: note = synthetic sequence
  VARIANT 9: note = X is F, Y, S, or T
  VARIANT 44: note = X is K or R
  VARIANT 62: note = X is Q, G, E, or R
  VARIANT 63: note = X is N or E
  VARIANT 65: note = X is R or G
  VARIANT 66: note = X is N or K
  VARIANT 67: note = X is M or V
  VARIANT 70: note = X is H or Q
  VARIANT 73: note = X is H or Q
  VARIANT 74: note = X is D or H
  VARIANT 76: note = X is A, V, or E
  VARIANT 77: note = X is N or D
  VARIANT 79: note = X is G or R
  VARIANT 80: note = X is T or I
  VARIANT 81: note = X is L or A
  VARIANT 82: note = X is R or L
  VARIANT 83: note = X is G or R
  VARIANT 90: note = X is A or D
  VARIANT 95: note = X is I, L, or V
  VARIANT 97: note = X is I, R or M
  VARIANT 99: note = X is F or Y
  VARIANT 105: note = X is S or P
  VARIANT 107: note = X is W or G
  VARIANT 114: note = X is R, H, or Q
  VARIANT 116: note = X is D or Y
  VARIANT 127: note = X is N or K
  VARIANT 142: note = X is T or I
  VARIANT 144: note = X is K or Q
  VARIANT 145: note = X is R or H
  VARIANT 149: note = X is A or T
  VARIANT 150: note = X is A or V
  VARIANT 151: note = X is H or R
  VARIANT 156: note = X is R, L, Q, or W
  VARIANT 158: note = X is V or A
  VARIANT 161: note = X is D or E
  VARIANT 163: note = X is R or T
  VARIANT 166: note = X is D or E
  VARIANT 167: note = X is W or G
  VARIANT 184: note = X is P or A
  VARIANT 193: note = X is P or A
  VARIANT 194: note = X is P or A
  VARIANT 207: note = X is S or G
  VARIANT 246: note = X is A or S
  VARIANT 253: note = X is Q or E
  VARIANT 276: note = X is P or L
GSHSMRYFXTSVSRPGRGEPRFIAVGYVDDTQFVRFDSDAASQXMEPRAPWIEQEGPEYWDXXTXXXKAXSQXXRXXLXXXXXYYNQSEXGSHTXQXMXGCDVGXDXRFLRGYXQXAYDGKDYIALXEDLRSWTAADMAAQXTXXKWEXXXEAEQXRXYLXGXCVXXLRRYLENGKETLQRTDXPKTHMTHHXXSDHEATLRCWALXFYPAEITLTWQRDGEDQTQDTELVETRPAGDGTFQKWAXVVVPSGXEQRYTCHVQHEGLPKPLTLRWEX

SEQ ID NO: 46 - protein, 276 aa, Homo sapiens [sequence omitted]

SEQ ID NO: 47 - protein, 276 aa, Homo sapiens
GSHSMRYFDTAMSRPGRGEPRFISVGYVDDTQFVRFDSDAASPREEPRAPWIEQEGPEYWDRNTQIFKTNTQTDRESLRNLRGYYNQSEAGSHTLQSMYGCDVGPDGRLLRGHNQYAYDGKDYIALNEDLRSWTAADTAAQITQRKWEAARVAEQDRAYLEGTCVEWLRRYLENGKDTLERADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 48 - protein, 276 aa, Homo sapiens
GSHSMRYFYTAMSRPGRGEPRFIAVGYVDDTQFVRFDSDAASPRMAPRAPWIEQEGPEYWDRNTQISKTNTQTYRESLRNLRGYYNQSEAGSHIIQRMYGCDVGPDGRLLRGYDQSAYDGKDYIALNEDLSSWTAADTAAQITQRKWEAAREAEQLRAYLEGLCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 49 - protein, 276 aa, Homo sapiens
GSHSMRYFYTSVSRPGRGEPRFISVGYVDDTQFVRFDSDAASPREEPRAPWIEQEGPEYWDRNTQICKTNTQTYRENLRTALRYYNQSEAGSHTLQRMYGCDVGPDGRLLRGHNQFAYDGKDYIALNEDLSSWTAADTAAQITQRKWEAARVAEQLRTYLEGTCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 50 - protein, 276 aa, Homo sapiens
GSHSMRYFHTAMSRPGRGEPRFITVGYVDDTLFVRFDSDATSPRKEPRAPWIEQEGPEYWDRETQISKTNTQTYRESLRNLRGYYNQSEAGSHTLQRMYGCDVGPDGRLLRGHNQYAYDGKDYIALNEDLRSWTAADTAAQISQRKLEAARVAEQLRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 51 - protein, 276 aa, Homo sapiens
GSHSMRYFYTAMSRPGRGEPRFIAVGYVDDTQFVRFDSDAASPRMAPRAPWIEQEGPEYWDRETQKYKRQAQTDRVSLRNLRGYYNQSEAGSHTLQRMYGCDVGPDGRLLRGHDQSAYDGKDYIALNEDLSSWTAADTAAQITQRKWEAAREAEQWRAYLEGLCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 52 - protein, 276 aa, Homo sapiens
GSHSMRYFYTAMSRPGRGEPRFIAVGYVDDTQFVRFDSDAASPRTEPRAPWIEQEGPEYWDRNTQIFKTNTQTYRENLRIALRYYNQSEAGSHIIQRMYGCDLGPDGRLLRGHDQSAYDGKDYIALNEDLSSWTAADTAAQITQRKWEAARVAEQLRAYLEGLCVEWLRRYLENGKETLQRADPPKTHVTHHPVSDHEATLRCWALGFYPAEITLTWQRDGEDQTQDTELVETRPAGDRTFQKWAAVVVPSGEEQRYTCHVQHEGLPKPLTLRWEP

SEQ ID NO: 53 - protein, 276 aa, synthetic construct
  REGION 1..276: note = synthetic sequence
  VARIANT 9: note = X is H, Y or D
  VARIANT 11: note = X is A or S
  VARIANT 12: note = X is M or V
  VARIANT 24: note = X is A, S, or T
  VARIANT 32: note = X is Q or L
  VARIANT 41: note = X is A or T
  VARIANT 45: note = X is E, M K, or T
  VARIANT 46: note = X is A or T
  VARIANT 63: note = X is E or N
  VARIANT 66: note = X iis I or K
  VARIANT 67: note = X is Y, F, S, or C
  VARIANT 70: note = X is N or Q
  VARIANT 71: note = X is A or T
  VARIANT 74: note = X is D or Y
  VARIANT 76: note = X is E or V
  VARIANT 77: note = X is S or N
  VARIANT 80: note = X is T, N or I
  VARIANT 81: note = X is A or L
  VARIANT 82: note = X is L or R
  VARIANT 83: note = X is R or G
  VARIANT 94: note = X is T or I
  VARIANT 95: note = X is L or I
  VARIANT 97: note = X is R or S
  VARIANT 131: note = X is R or S
  VARIANT 143: note = X is S or T
  VARIANT 147: note = X is L or W
  VARIANT 152: note = X is E or V
  VARIANT 156: note = X is R, D, L or W
  VARIANT 158: note = X is A or T
  VARIANT 163: note = X is L, E or T
  VARIANT 177: note = X is E or D
  VARIANT 178: note = X is K or T
  VARIANT 180: note = X is E or Q
  VARIANT 194: note = X is I or V
GSHSMRYFXTXXSRPGRGEPRFIXVGYVDDTXFVRFDSDAXSPRXXPRAPWIEQEGPEYWDRXTQXXKTXXTQXYXXNLXXXXYYNQSEAGSHXXQXMYGCDLGPDGRLLRGHDQSAYDGKDYIALNEDLXSWTAADTAAQIXQRKXEAARXAEQXRXYLEGXCVEWLRRYLENGKXXLXRADPPKTHVTHHPXSDHEATLRCWALGFYPAEITLTWQRDGEDQTQDTELVETRPAGDRTFQKWAAVVVPSGEEQRYTCHVQHEGLPKPLTLRWEP

SEQ ID NO: 54 - protein, 276 aa, Homo sapiens
CSHSMKYFFTSVSRPGRGEPRFISVGYVDDTQFVRFDSDAASPRGEPRAPWVEQEGPEYWDRETQKYKRQAQTDRVSLRNLRGYYNQSEAGSHTLQWMCGCDLGPDGRLLRGYDQYAYDGKDYIALNEDLRSWTAADTAAQITQRKWEAAREAEQRRAYLEGTCVEWLRRYLENGKETLQRAEHPKTHVTHHPVSDHEATLRCWALGFYPAEITLTWQWDGEDQTQDTELVETRPAGDGTFQKWAAVMVPSGEEQRYTCHVQHEGLPEPLTLRWEP

SEQ ID NO: 55 - protein, 276 aa, Homo sapiens
GSHSMRYFYTAVSRPGRGEPHFIAVGYVDDTQFVRFDSDAASPRGEPRAPWVEQEGPEYWDRETQKYKRQAQTDRVSLRNLRGYYNQSEARSHIIQRMYGCDVGPDGRLLRGYDQYAYDGKDYIALNEDLRSWTAADTAAQITQRKWEAAREAEQLRAYLEGLCVEWLRRYLKNGKETLQRAEHPKTHVTHHPVSDHEATLRCWALGFYPAEITLTWQWDGEDQTQDTELVETRPAGDGTFQKWAAVVVPSGEEQRYTCHVQHEGLPEPLTLRWEP

SEQ ID NO: 56 - protein, 276 aa, Homo sapiens
GSHSMRYFYTAVSRPGRGEPHFIAVGYVDDTQFVRFDSDAASPRGEPRAPWVEQEGPEYWDRETQKYKRQAQTDRVSLRNLRGYYNQSEAGSHIIQRMYGCDVGPDGRLLRGYDQYAYDGKDYIALNEDLRSWTAADTAAQITQRKWEAAREAEQLRAYLEGLCVEWLRRYLKNGKETLQRAEHPKTHVTHHPVSDHEATLRCWALGFYPAEITLTWQWDGEDQTQDTELVETRPAGDGTFQKWAAVVVPSGEEQRYTCHVQHEGLPEPLTLRWEP

SEQ ID NO: 57 - protein, 276 aa, Homo sapiens
GSHSMRYFSTSVSWPGRGEPRFIAVGYVDDTQFVRFDSDAASPRGEPREPWVEQEGPEYWDRETQKYKRQAQADRVNLRKLRGYYNQSEDGSHTLQRMFGCDLGPDGRLLRGYNQFAYDGKDYIALNEDLRSWTAADTAAQITQRKWEAAREAEQRRAYLEGTCVEWLRRYLENGKETLQRAEHPKTHVTHHPVSDHEATLRCWALGFYPAEITLTWQWDGEDQTQDTELVETRPAGDGTFQKWAAVVVPSGEEQRYTCHVQHEGLPEPLTLRWKP

SEQ ID NO: 58 - protein, 276 aa, Homo sapiens
CSHSMRYFDTAVSRPGRGEPRFISVGYVDDTQFVRFDSDAASPRGEPRAPWVEQEGPEYWDRETQKYKRQAQADRVNLRKLRGYYNQSEDGSHTLQWMYGCDLGPDGRLLRGYDQSAYDGKDYIALNEDLRSWTAADTAAQITQRKWEAAREAEQWRAYLEGTCVEWLRRYLENGKETLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWEP

SEQ ID NO: 59 - protein, 276 aa, Homo sapiens
CSHSMRYFDTAVSRPGRGEPRFISVGYVDDTQFVRFDSDAASPRGEPRAPWVEQEGPEYWDRETQKYKRQAQADRVSLRNLRGYYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEP

SEQ ID NO: 60 - protein, 276 aa, Homo sapiens
CSHSMRYFYTAVSRPGRGEPRFIAVGYVDDTQFVQFDSDAASPRGEPRAPWVEQEGPEYWDRETQKYKRQAQTDRVSLRNLRGYYNQSEAGSHTLQRMYGCDLGPDGRLLRGYNQFAYDGKDYIALNEDLRSWTAADTAAQITQRKWEAARTAEQLRAYLEGTCVEWLRRYLENGKKTLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWGP

SEQ ID NO: 61 - protein, 276 aa, Homo sapiens
CSHSMRYFYTAVSRPGRGEPHFIAVGYVDDTQFVRFDSDAASPRGEPRAPWVEQEGPEYWDRETQNYKRQAQTDRVNLRKLRGYYNQSEAGSHIIQRMYGCDLGPDGRLLRGHDQLAYDGKDYIALNEDLRSWTAADTAAQITQRKWEAAREAEQLRAYLEGTCVEWLRRYLENGKETLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWEP

SEQ ID NO: 62 - protein, 276 aa, synthetic construct
  REGION 1..276: note = synthetic sequence
  VARIANT 1: note = X is C or G
  VARIANT 6: note = X is R or K
  VARIANT 9: note = X is F, Y, S, or D
  VARIANT 14: note = X is R or W
  VARIANT 21: note = X is H or R
  VARIANT 24: note = X is A or S
  VARIANT 35: note = X is Q or R
  VARIANT 49: note = X is A or E
  VARIANT 66: note = X is N or K
  VARIANT 73: note = X is T or A
  VARIANT 77: note = X is S or N
  VARIANT 80: note = X is N or K
  VARIANT 90: note = X is A or D
  VARIANT 91: note = X is G or R
  VARIANT 94: note = X is T or I
  VARIANT 95: note = X is L or I
  VARIANT 97: note = X is W or R
  VARIANT 99: note = X is C, Y, F, or S
  VARIANT 103: note = X is L or V
  VARIANT 113: note = X is Y or H
  VARIANT 114: note = X is D or N
  VARIANT 116: note = X is Y, F, S, or L
  VARIANT 147: note = X is L or W
  VARIANT 152: note = X is E, A or T
  VARIANT 156: note = X is R, L or W
  VARIANT 163: note = X is L or T
  VARIANT 173: note = X is E or K
  VARIANT 177: note = X is E or K
  VARIANT 184: note = X is H or P
  VARIANT 194: note = X is R or V
  VARIANT 219: note = X is W or R
  VARIANT 248: note = X is V or M
  VARIANT 253: note = X is E or Q
  VARIANT 261: note = X is M or V
  VARIANT 267: note = X is P or Q
  VARIANT 273: note = X is R or S
  VARIANT 275: note = X is P or G
XSHSMXYFXTAVSXPGRGEPXFIXVGYVDDTQFVXFDSDAASPRGEPRXPWVEQEGPEYWDRETQXYKRQAQXDRVXLRXLRGYYNQSEXXSHXXQXMXGCDXGPDGRLLRGXXQXAYDGKDYIALNEDLRSWTAADTAAQITQRKXEAARXAEQXRAYLEGXCVEWLRRYLXNGKXTLQRAEXPKTHVTHHPXSDHEATLRCWALGFYPAEITLTWQXDGEDQTQDTELVETRPAGDGTFQKWAAVXVPSGXEQRYTCHXQHEGLXEPLTLXWXP

SEQ ID NO: 63 - protein, 286 aa, synthetic construct
  REGION 1..286: note = synthetic sequence
  VARIANT 89: note = X is K or E
  VARIANT 108: note = X is R or G
  VARIANT 158: note = X is R or G
  VARIANT 159: note = X is A or V
  VARIANT 256: note = X is Q or P
  VARIANT 269: note = X is P or S
GSHSLKYFHTSVSRPGRGEPRFISVGYVDDTQFVRFDNDAASPRMVPRAPWMEQEGSEYWDRETRSARDTAQIFRVNLRTLRGYYNQSXLAGSHTLQWMHGCELGPDXRFLRGYEQFAYDGKDYLTLNEDLRSWTAVDTAAQISEQKSNDASEAEHQXXYLEDTCVEWLHKYLEKGKETLLHLEPPKTHVTHHPISDHEATLRCWALGFYPAEITLTWQQDGEGHTQDTELVETRPAGDGTFQKWAAVVVPSGEEXSRYTCHVQHEGLXEPVTLRWKPASQPTIPI

SEQ ID NO: 64 - protein, 285 aa, synthetic construct
  REGION 1..285: note = synthetic sequence
  VARIANT 7: note = X is Y or F
  VARIANT 51: note = X is P or Q
  VARIANT 252: note = X is S or P
  VARIANT 279: note = X is P or L
GSHSLRXLFSTAVSRPGRGEPRYIAVEYVDDTQFLRFDSDAAIPRMEPREXWVEQEGPQYWEWTTGYAKANAQTDRVALRNLLRRYNQSEAGSHTLQGMNGCDMGPDGRLLRGYHQHAYDGKDYISLNEDLRSWTAADTVAQITQRFYEAEEYAEEFRTYLEGECLELLRRYLENGKETLQRADPPKAHVAHHPISDHEATLRCWALGFYPAEITLTWQRDGEEQTQDTELVETRPAGDGTFQKWAAVVVPXGEEQRYTCHVQHEGLPQPLILRWEQSXQPTIPI

SEQ ID NO: 65 - protein, 286 aa, synthetic construct
  REGION 1..286: note = synthetic sequence
  VARIANT 13: note = X is S or F
  VARIANT 27: note = X is Y or H
  VARIANT 31: note = X is T, S or M
  VARIANT 34: note = X is L or V
  VARIANT 54: note = X is Q or R
  VARIANT 82: note = X is P or L
  VARIANT 101: note = X is G or D
  VARIANT 105: note = X is G or V
  VARIANT 106: note = X is S or C
  VARIANT 111: note = X is L or I
  VARIANT 160: note = X is Y or H
  VARIANT 170: note = X is H or R
  VARIANT 172: note = X is Y or H
  VARIANT 179: note = X is M or T
  VARIANT 186: note = X is P or A
  VARIANT 221: note = X is R, W or Q
  VARIANT 260: note = X is T or M
  VARIANT 277: note = X is K or E
GSHSMRYFSAAVXRPGRGEPRFIAMGXVDDXQFXRFDSDSACPRMEPRAPWVEXSEGPEYWEEETRNTKAHAQTDRMNLQTXRGYYNQSEASSHTLQWMIXCDLXXDGRLXRGYEQYAYDGKDYLALNEDLRSWTAADTAAQISKRKCEAANVAEQRRAXLEGTCVEWLXRXLENGKEXLQRADPXSKTHVTHHPVFDYEATLRCWALGFYPAEIILTWQXDGEDQTQDVELVETRPAGDGTFQKWAAVVVPSGEEQRYXCHVQHEGLPEPLMLRWXQSSLPTIPI

SEQ ID NO: 66 - protein, 133 aa, Homo sapiens [sequence omitted]

SEQ ID NO: 67 - protein, 272 aa, synthetic construct
MDSYLLMWGLLTFIMVPGCQAELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 68 - protein, 551 aa, synthetic construct
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 69 - protein, 369 aa, synthetic construct
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 70 - protein, 306 aa, Severe acute respiratory syndrome coronavirus 2 [sequence omitted]

SEQ ID NO: 71 - protein, 75 aa, Severe acute respiratory syndrome coronavirus 2
MYSFVSEETGTLIVNSVLLFLAFVVFLLVTLAILTALRLCAYCCNIVNVSLVKPSFYVYSRVKNLNSSRVPDLLV

SEQ ID NO: 72 - protein, 601 aa, Severe acute respiratory syndrome coronavirus 2
AVGACVLCNSQTSLRCGACIRRPFLCCKCCYDHVISTSHKLVLSVNPYVCNAPGCDVTDVTQLYLGGMSYYCKSHKPPISFPLCANGQVFGLYKNTCVGSDNVTDFNAIATCDWTNAGDYILANTCTERLKLFAAETLKATEETFKLSYGIATVREVLSDRELHLSWEVGKPRPPLNRNYVFTGYRVTKNSKVQIGEYTFEKGDYGDAVVYRGTTTYKLNVGDYFVLTSHTVMPLSAPTLVPQEHYVRITGLYPTLNISDEFSSNVANYQKVGMQKYSTLQGPPGTGKSHFAIGLALYYPSARIVYTACSHAAVDALCEKALKYLPIDKCSRIIPARARVECFDKFKVNSTLEQYVFCTVNALPETTADIVVFDEISMATNYDLSVVNARLRAKHYVYIGDPAQLPAPRTLLTKGTLEPEYFNSVCRLMKTIGPDMFLGTCRRCPAEIVDTVSALVYDNKLKAHKDKSAQCFKMFYKGVITHDVSSAINRPQIGVVREFLTRNPAWRKAVFISPYNSQNAVASKILGLPTQTVDSSQGSEYDYVIFTQTTETAHSCNVNRFNVAITRAKVGILCIMSDRDLYDKLQFTSLEIPRRNVATLQ

SEQ ID NO: 73 - protein, 222 aa, Severe acute respiratory syndrome coronavirus 2
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 74 - protein, 180 aa, Severe acute respiratory syndrome coronavirus 2
MESLVPGFNEKTHVQLSLPVLQVRDVLVRGFGDSVEEVLSEARQHLKDGTCGLVEVEKGVLPQLEQPYVFIKRSDARTAPHGHVMVELVAELEGIQYGRSGETLGVLVPHVGEIPVAYRKVLLRKNGNKGAGGHSYGADLKSFDLGDELGTDPYEDFQENWNTKHSSGVTRELMRELNGG

SEQ ID NO: 75 - protein, 638 aa, Severe acute respiratory syndrome coronavirus 2
AYTRYVDNNFCGPDGYPLECIKDLLARAGKASCTLSEQLDFIDTKRGVYCCREHEHEIAWYTERSEKSYELQTPFEIKLAKKFDTFNGECPNFVFPLNSIIKTIQPRVEKKKLDGFMGRIRSVYPVASPNECNQMCLSTLMKCDHCGETSWQTGDFVKATCEFCGTENLTKEGATTCGYLPQNAVVKIYCPACHNSEVGPEHSLAEYHNESGLKTILRKGGRTIAFGGCVFSYVGCHNKCAYWVPRASANIGCNHTGVVGEGSEGLNDNLLEILQKEKVNINIVGDFKLNEEIAIILASFSASTSAFVETVKGLDYKAFKQIVESCGNFKVTKGKAKKGAWNIGEQKSILSPLYAFASEAARVVRSIFSRTLETAQNSVRVLQKAAITILDGISQYSLRLIDAMMFTSDLATNNLVVMAYITGGVVQLTSQWLTNIFGTVYEKLKPVLDWLEEKFKEGVEFLRDGWEIVKFISTCACEIVGGQIVTCAKEIKESVQTFFKLVNKFLALCADSIIIGGAKLKALNLGETFVTHSKGLYRKCVKSREETGLLMPLKAPKEIIFLEGETLPTEVLTEEVVLKTGDLQPLEQPTSEAVEAPLVGTPVCINGLMLLEIKDTEKYCALAPNMMVTNNTFTLKGG

SEQ ID NO: 76 - protein, 500 aa, Severe acute respiratory syndrome coronavirus 2
KIVNNWLKQLIKVTLVFLFVAAIFYLITPVHVMSKHTDFSSEIIGYKAIDGGVTRDIASTDTCFANKHADFDTWFSQRGGSYTNDKACPLIAAVITREVGFVVPGLPGTILRTTNGDFLHFLPRVFSAVGNICYTPSKLIEYTDFATSACVLAAECTIFKDASGKPVPYCYDTNVLEGSVAYESLRPDTRYVLMDGSIIQFPNTYLEGSVRVVTTFDSEYCRHGTCERSEAGVCVSTSGRWVLNNDYYRSLPGVFCGVDAVNLLTNMFTPLIQPIGALDISASIVAGGIVAIVVTCLAYYFMRFRRAFGEYSHVVAFNTLLFLMSFTVLCLTPVYSFLPGVYSVIYLYLTFYLTNDVSFLAHIQWMVMFTPLVPFWITIAYIICISTKHFYWFFSNYLKRRVVFNGVSFSTFEEAALCTFLLNKEMYLKLRSDVLLPLTQYNRYLALYNKYKYFSGAMDTTSYREAACCHLAKALNDFSNSGSDVLYQPPQTSITSAVLQ

SEQ ID NO: 77 - protein, 290 aa, Severe acute respiratory syndrome coronavirus 2
SAVKRTIKGTHHWLLLTILTSLLVLVQSTQWSLFFFLYENAFLPFAMGIIAMSAFAMMFVKHKHAFLCLFLLPSLATVAYFNMVYMPASWVMRIMTWLDMVDTSLSGFKLKDCVMYASAVVLLILMTARTVYDDGARRVWTLMNVLTLVYKVYYGNALDQAISMWALIISVTSNYSGVVTTVMFLARGIVFMCVEYCPIFFITGNTLQCIMLVYCFLGYFCTCYFGLFCLLNRYFRLTLGVYDYLVSTQEFRYMNSQGLLPPKNSIDAFKLNIKLLGVGGKPCIKVATVQ

SEQ ID NO: 78 - protein, 83 aa, Severe acute respiratory syndrome coronavirus 2
SKMSDVKCTSVVLLSVLQQLRVESSSKLWAQCVQLHNDILLAKDTTEAFEKMVSLLSVLLSMQGAVDINKLCEEMLDNRATLQ

SEQ ID NO: 79 - protein, 198 aa, Severe acute respiratory syndrome coronavirus 2
AIASEFSSLPSYAAFATAQEAYEQAVANGDSEVVLKKLKKSLNVAKSEFDRDAAMQRKLEKMADQAMTQMYKQARSEDKRAKVTSAMQTMLFTMLRKLDNDALNNIINNARDGCVPLNIIPLTTAAKLMVVIPDYNTYKNTCDGTTFTYASALWEIQQVVDADSKIVQLSEISMDNSPNLAWPLIVTALRANSAVKLQ

SEQ ID NO: 80 - protein, 113 aa, Severe acute respiratory syndrome coronavirus 2
NNELSPVALRQMSCAAGTTQTACTDDNALAYYNTTKGGRFVLALLSDLQDLKWARFPKSDGTGTIYTELEPPCRFVTDTPKGPKVKYLYFIKGLNNLNRGMVLGSLAATVRLQ

SEQ ID NO: 81 - protein, 139 aa, Severe acute respiratory syndrome coronavirus 2
AGNATEVPANSTVLSFCAFAVDAAKAYKDYLASGGQPITNCVKMLCTHTGTGQAITVTPEANMDQESFGGASCCLYCRCHIDHPNPKGFCDLKGKYVQIPTTCANDPVGFTLKNTVCTVCGMWKGYGCSCDQLREPMLQ

SEQ ID NO: 82 - protein, 527 aa, Severe acute respiratory syndrome coronavirus 2
AENVTGLFKDCSKVITGLHPTQAPTHLSVDTKFKTEGLCVDIPGIPKDMTYRRLISMMGFKMNYQVNGYPNMFITREEAIRHVRAWIGFDVEGCHATREAVGTNLPLQLGFSTGVNLVAVPTGYVDTPNNTDFSRVSAKPPPGDQFKHLIPLMYKGLPWNVVRIKIVQMLSDTLKNLSDRVVFVLWAHGFELTSMKYFVKIGPERTCCLCDRRATCFSTASDTYACWHHSIGFDYVYNPFMIDVQQWGFTGNLQSNHDLYCQVHGNAHVASCDAIMTRCLAVHECFVKRVDWTIEYPIIGDELKINAACRKVQHMVVKAALLADKFPVLHDIGNPKAIKCVPQADVEWKFYDAQPCSDKAYKIEELFYSYATHSDKFTDGVCLFWNCNVDRYPANSIVCRFDTRVLSNLNLPGCDGGSLYVNKHAFHTPAFDKSAFVNLKQLPFFYYSDSPCESHGKQVVSDIDYVPLKSATCITRCNLGGAVCRHHANEYRLYLDAYNMMISAGFSLWVYKQFDTYNLWNTFTRLQ

SEQ ID NO: 83 - protein, 346 aa, Severe acute respiratory syndrome coronavirus 2
SLENVAFNVVNKGHFDGQQGEVPVSIINNTVYTKVDGVDVELFENKTTLPVNVAFELWAKRNIKPVPEVKILNNLGVDIAANTVIWDYKRDAPAHISTIGVCSMTDIAKKPTETICAPLTVFFDGRVDGQVDLFRNARNGVLITEGSVKGLQPSVGPKQASLNGVTLIGEAVKTQFNYYKKVDGVVQQLPETYFTQSRNLQEFKPRSQMEIDFLELAMDEFIERYKLEGYAFEHIVYGDFSHSQLGGLHLLIGLAKRFKESPFELEDFIPMDSTVKNYFITDAQTGSSKCVCSVIDLLLDDFVEIIKSQDLSVVSKVVKVTIDYTEISFMLWCKDGHVETFYPKLQ

SEQ ID NO: 84 - protein, 298 aa, Severe acute respiratory syndrome coronavirus 2
SSQAWQPGVAMPNLYKMQRMLLEKCDLQNYGDSATLPKGIMMNVAKYTQLCQYLNTLTLAVPYNMRVIHFGAGSDKGVAPGTAVLRQWLPTGTLLVDSDLNDFVSDADSTLIGDCATVHTANKWDLIISDMYDPKTKNVTKENDSKEGFFTYICGFIQQKLALGGSVAIKITEHSWNADLYKLMGHFAWWTAFVTNVNASSSEAFLIGCNYLGKPREQIDGYVMHANYIFWRNTNPIQLSSYSLFDMSKFPLKLRGTAVMSLKEGQINDMILSLLSKGRLIIRENNRVVISSDVLVNN

SEQ ID NO: 85 - protein, 419 aa, Severe acute respiratory syndrome coronavirus 2
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 86 - protein, 275 aa, Severe acute respiratory syndrome coronavirus 2
MDLFMRIFTIGTVTLKQGEIKDATPSDFVRATATIPIQASLPFGWLIVGVALLAVFQSASKIITLKKRWQLALSKGVHFVCNLLLLFVTVYSHLLLVAAGLEAPFLYLYALVYFLQSINFVRIIMRLWLCWKCRSKNPLLYDANYFLCWHTNCYDYCIPYNSVTSSIVITSGDGTTSPISEHDYQIGGYTEKWESGVKDCVVLHSYFTSDYYQLYSTQLSTDTGVEHVTFFIYNKIVDEPEEHVQIHTIDGSSGVVNPVMEPIYDEPTTTTSVPL

SEQ ID NO: 87 - protein, 61 aa, Severe acute respiratory syndrome coronavirus 2
MFHLVDFQVTIAEILLIIMRTFKVSIWNLDYIINLIIKNLSKSLTENKYSQLDEEQPMEID

SEQ ID NO: 88 - protein, 121 aa, Severe acute respiratory syndrome coronavirus 2
MKIILFLALITLATCELYHYQECVRGTTVLLKEPCSSGTYEGNSPFHPLADNKFALTCFSTQFAFACPDGVKHVYQLRARSVSPKLFIRQEEVQELYSPIFLIVAAIVFITLCFTLKRKTE

SEQ ID NO: 89 - protein, 121 aa, Severe acute respiratory syndrome coronavirus 2
MKFLVFLGIITTVAAFHQECSLQSCTQHQPYVVDDPCPIHFYSKWYIRVGARKSAPLIELCVDEAGSKSPIQYIDIGNYTVSCLPFTINCQEPKLGSLVVRCSFYEDFLEYHDVRVVLDFI

SEQ ID NO: 90 - protein, 38 aa, Severe acute respiratory syndrome coronavirus 2
MGYINVFAFPFTIYSLLLCRMNSRNYIAQVDVVNFNLT

SEQ ID NO: 91 - protein, 1945 aa, Severe acute respiratory syndrome coronavirus 2
APTKVTFGDDTVIEVQGYKSVNITFELDERIDKVLNEKCSAYTVELGTEVNEFACVVADAVIKTLQPVSELLTPLGIDLDEWSMATYYLFDESGEFKLASHMYCSFYPPDEDEEEGDCEEEEFEPSTQYEYGTEDDYQGKPLEFGATSAALQPEEEQEEDWLDDDSQQTVGQQDGSEDNQTTTIQTIVEVQPQLEMELTPVVQTIEVNSFSGYLKLTDNVYIKNADIVEEAKKVKPTVVVNAANVYLKHGGGVAGALNKATNNAMQVESDDYIATNGPLKVGGSCVLSGHNLAKHCLHVVGPNVNKGEDIQLLKSAYENFNQHEVLLAPLLSAGIFGADPIHSLRVCVDTVRTNVYLAVFDKNLYDKLVSSFLEMKSEKQVEQKIAEIPKEEVKPFITESKPSVEQRKQDDKKIKACVEEVTTTLEETKFLTENLLLYIDINGNLHPDSATLVSDIDITFLKKDAPYIVGDVVQEGVLTAVVIPTKKAGGTTEMLAKALRKVPTDNYITTYPGQGLNGYTVEEAKTVLKKCKSAFYILPSIISNEKQEILGTVSWNLREMLAHAEETRKLMPVCVETKAIVSTIQRKYKGIKIQEGVVDYGARFYFYTSKTTVASLINTLNDLNETLVTMPLGYVTHGLNLEEAARYMRSLKVPATVSVSSPDAVTAYNGYLTSSSKTPEEHFIETISLAGSYKDWSYSGQSTQLGIEFLKRGDKSVYYTSNPTTFHLDGEVITFDNLKTLLSLREVRTIKVFTTVDNINLHTQVVDMSMTYGQQFGPTYLDGADVTKIKPHNSHEGKTFYVLPNDDTLRVEAFEYYHTTDPSFLGRYMSALNHTKKWKYPQVNGLTSIKWADNNCYLATALLTLQQIELKFNPPALQDAYYRARAGEAANFCALILAYCNKTVGELGDVRETMSYLFQHANLDSCKRVLNVVCKTCGQQQTTLKGVEAVMYMGTLSYEQFKKGVQIPCTCGKQATKYLVQQESPFVMMSAPPAQYELKHGTFTCASEYTGNYQCGHYKHITSKETLYCIDGALLTKSSEYKGPITDVFYKENSYTTTIKPVTYKLDGVVCTEIDPKLDNYYKKDNSYFTEQPIDLVPNQPYPNASFDNFKFVCDNIKFADDLNQLTGYKKPASRELKVTFFPDLNGDVVAIDYKHYTPSFKKGAKLLHKPIVWHVNNATNKATYKPNTWCIRCLWSTKPVETSNSFDVLKSEDAQGMDNLACEDLKPVSEEVVENPTIQKDVLECNVKTTEVVGDIILKPANNSLKITEEVGHTDLMAAYVDNSSLTIKKPNELSRVLGLKTLATHGLAAVNSVPWDTIANYAKPFLNKVVSTTTNIVTRCLNRVCTNYMPYFFTLLLQLCTFTRSTNSRIKASMPTTIAKNTVKSVGKFCLEASFNYLKSPNFSKLINIIIWFLLLSVCLGSLIYSTAALGVLMSNLGMPSYCTGYREGYLNSTNVTIATYCTGSIPCSVCLSGLDSLDTYPSLETIQITISSFKWDLTAFGLVAEWFLAYILFTRFFYVLGLAAIMQLFFSYFAVHFISNSWLMWLIINLVQMAPISAMVRMYIFFASFYYVWKSYVHVVDGCNSSTCMMCYKRNRATRVECTTIVNGVRRSFYVYANGGKGFCKLHNWNCVNCDTFCAGSTFISDEVARDLSLQFKRPINPTDQSSYIVDSVTVKNGSIHLYFDKAGQKTYERHSLSHFVNLDNLRANNTKGSLPINVIVFDGKSKCEESSAKSASVYYSQLMCQPILLLDQALVSDVGDSAEVAVKMFDAYVNTFSSTFNVPMEKLKTLVATAEAELAKNVSLDNVLSTFISAARQGFVDSDVETKDVVECLKLSHQSDIEVTGDSCNNYMLTYNKVENMTPRDLGACIDCSARHINAQVAKSHNIALIWNVKDFMSLSEQLRKQIRSAAKKNNLPFKLTCATTRQVVNVVTTKIALKGG

SEQ ID NO: 92 - protein, 932 aa, Severe acute respiratory syndrome coronavirus 2
SADAQSFLNRVCGVSAARLTPCGTGTSTDVVYRAFDIYNDKVAGFAKFLKTNCCRFQEKDEDDNLIDSYFVVKRHTFSNYQHEETIYNLLKDCPAVAKHDFFKFRIDGDMVPHISRQRLTKYTMADLVYALRHFDEGNCDTLKEILVTYNCCDDDYFNKKDWYDFVENPDILRVYANLGERVRQALLKTVQFCDAMRNAGIVGVLTLDNQDLNGNWYDFGDFIQTTPGSGVPVVDSYYSLLMPILTLTRALTAESHVDTDLTKPYIKWDLLKYDFTEERLKLFDRYFKYWDQTYHPNCVNCLDDRCILHCANFNVLFSTVFPPTSFGPLVRKIFVDGVPFVVSTGYHFRELGVVHNQDVNLHSSRLSFKELLVYAADPAMHAASGNLLLDKRTTCFSVAALTNNVAFQTVKPGNFNKDFYDFAVSKGFFKEGSSVELKHFFFAQDGNAAISDYDYYRYNLPTMCDIRQLLFVVEVVDKYFDCYDGGCINANQVIVNNLDKSAGFPFNKWGKARLYYDSMSYEDQDALFAYTKRNVIPTITQMNLKYAISAKNRARTVAGVSICSTMTNRQFHQKLLKSIAATRGATVVIGTSKFYGGWHNMLKTVYSDVENPHLMGWDYPKCDRAMPNMLRIMASLVLARKHTTCCSLSHRFYRLANECAQVLSEMVMCGGSLYVKPGGTSSGDATTAYANSVFNICQAVTANVNALLSTDGNKIADKYVRNLQHRLYECLYRNRDVDTDFVNEFYAYLRKHFSMMILSDDAVVCFNSTYASQGLVASIKNFKSVLYYQNNVFMSEAKCWTETDLTKGPHEFCSQHTMLVKQGDDYVYLPYPDPSRILGAGCFVDDIVKTDGTLMIERFVSLAIDAYPLTKHPNQEYADVFHLYLQYIRKLHDELTGHMLDMYSVMLTNDNTSRYWEPEFYEAMYTPHTVLQ

SEQ ID NO: 93 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 94 - protein, 1270 aa, synthetic construct
MFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFSTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 95 - protein, 10 aa, Severe acute respiratory syndrome coronavirus 2
TSEDMLNPNY

SEQ ID NO: 96 - protein, 14 aa, Severe acute respiratory syndrome coronavirus 2
TGVHAGTDLEGNFY

SEQ ID NO: 97 - protein, 15 aa, Severe acute respiratory syndrome coronavirus 2
DVTDVTQLYLGGMSY

SEQ ID NO: 98 - protein, 10 aa, Severe acute respiratory syndrome coronavirus 2
ATEETFKLSY

SEQ ID NO: 99 - protein, 12 aa, Severe acute respiratory syndrome coronavirus 2
ISDEFSSNVANY

SEQ ID NO: 100 - protein, 10 aa, Severe acute respiratory syndrome coronavirus 2
TVDSSQGSEY

SEQ ID NO: 101 - protein, 14 aa, Severe acute respiratory syndrome coronavirus 2
ASQRVAGDSGFAAY

SEQ ID NO: 102 - protein, 10 aa, Severe acute respiratory syndrome coronavirus 2
VAGDSGFAAY

SEQ ID NO: 103 - protein, 10 aa, Severe acute respiratory syndrome coronavirus 2
LAMDEFIERY

SEQ ID NO: 104 - protein, 11 aa, Severe acute respiratory syndrome coronavirus 2
ITEHSWNADLY

SEQ ID NO: 105 - protein, 12 aa, Severe acute respiratory syndrome coronavirus 2
YVDNNFCGPDGY

SEQ ID NO: 106 - protein, 9 aa, Severe acute respiratory syndrome coronavirus 2
FIDTKRGVY

SEQ ID NO: 107 - protein, 9 aa, Severe acute respiratory syndrome coronavirus 2
YTERSEKSY

SEQ ID NO: 108 - protein, 14 aa, Severe acute respiratory syndrome coronavirus 2
TSDLATNNLVVMAY

SEQ ID NO: 109 - protein, 11 aa, Severe acute respiratory syndrome coronavirus 2
HTDFSSEIIGY

SEQ ID NO: 110 - protein, 9 aa, Severe acute respiratory syndrome coronavirus 2
FSAVGNICY

SEQ ID NO: 111 - protein, 10 aa, Severe acute respiratory syndrome coronavirus 2
FSNSGSDVLY

SEQ ID NO: 112 - protein, 10 aa, Severe acute respiratory syndrome coronavirus 2
MADQAMTQMY

SEQ ID NO: 113 - protein, 14 aa, Severe acute respiratory syndrome coronavirus 2
NTYKNTCDGTTFTY

SEQ ID NO: 114 - protein, 14 aa, Severe acute respiratory syndrome coronavirus 2
TTQTACTDDNALAY

SEQ ID NO: 115 - protein, 13 aa, Severe acute respiratory syndrome coronavirus 2
QTACTDDNALAYY

SEQ ID NO: 116 - protein, 10 aa, Severe acute respiratory syndrome coronavirus 2
CTDDNALAYY

SEQ ID NO: 117 - protein, 14 aa, Severe acute respiratory syndrome coronavirus 2
WARFPKSDGTGTIY

SEQ ID NO: 118 - protein, 14 aa, Severe acute respiratory syndrome coronavirus 2
VTDTPKGPKVKYLY

SEQ ID NO: 119 - protein, 13 aa, Severe acute respiratory syndrome coronavirus 2
NTNSSPDDQIGYY

SEQ ID NO: 120 - protein, 10 aa, Severe acute respiratory syndrome coronavirus 2
YFTSDYYQLY

SEQ ID NO: 121 - protein, 14 aa, Severe acute respiratory syndrome coronavirus 2
STDTGVEHVTFFIY

SEQ ID NO: 122 - protein, 11 aa, Severe acute respiratory syndrome coronavirus 2
VVDDPCPIHFY

SEQ ID NO: 123 - protein, 8 aa, Severe acute respiratory syndrome coronavirus 2
LTENLLLY

SEQ ID NO: 124 - protein, 14 aa, Severe acute respiratory syndrome coronavirus 2
ALRKVPTDNYITTY

SEQ ID NO: 125 - protein, 11 aa, Severe acute respiratory syndrome coronavirus 2
KVPTDNYITTY

SEQ ID NO: 126 - protein, 9 aa, Severe acute respiratory syndrome coronavirus 2
VVDYGARFY

SEQ ID NO: 127 - protein, 11 aa, Severe acute respiratory syndrome coronavirus 2
HTTDPSFLGRY

SEQ ID NO: 128 - protein, 11 aa, Severe acute respiratory syndrome coronavirus 2
ITDVFYKENSY

SEQ ID NO: 129 - protein, 14 aa, Severe acute respiratory syndrome coronavirus 2
FTEQPIDLVPNQPY

SEQ ID NO: 130 - protein, 11 aa, Severe acute respiratory syndrome coronavirus 2
FADDLNQLTGY

SEQ ID NO: 131 - protein, 14 aa, Severe acute respiratory syndrome coronavirus 2
ITEEVGHTDLMAAY

SEQ ID NO: 132 - protein, 12 aa, Severe acute respiratory syndrome coronavirus 2
STDVVYRAFDIY

SEQ ID NO: 133 - protein, 11 aa, Severe acute respiratory syndrome coronavirus 2
FVENPDILRVY

SEQ ID NO: 134 - protein, 13 aa, Severe acute respiratory syndrome coronavirus 2
FVDGVPFVVSTGY

SEQ ID NO: 135 - protein, 10 aa, Severe acute respiratory syndrome coronavirus 2
AISDYDYYRY

SEQ ID NO: 136 - protein, 9 aa, Severe acute respiratory syndrome coronavirus 2
VVDKYFDCY

SEQ ID NO: 137 - protein, 14 aa, Severe acute respiratory syndrome coronavirus 2
YSDVENPHLMGWDY

SEQ ID NO: 138 - protein, 12 aa, Severe acute respiratory syndrome coronavirus 2
LSEMVMCGGSLY

SEQ ID NO: 139 - protein, 11 aa, Severe acute respiratory syndrome coronavirus 2
STDGNKIADKY

SEQ ID NO: 140 - protein, 13 aa, Severe acute respiratory syndrome coronavirus 2
NRDVDTDFVNEFY

SEQ ID NO: 141 - protein, 11 aa, Severe acute respiratory syndrome coronavirus 2
DTDFVNEFYAY

SEQ ID NO: 142 - protein, 13 aa, Severe acute respiratory syndrome coronavirus 2
LSDDAVVCFNSTY

SEQ ID NO: 143 - protein, 8 aa, Severe acute respiratory syndrome coronavirus 2
YADVFHLY

SEQ ID NO: 144 - protein, 13 aa, Severe acute respiratory syndrome coronavirus 2
YSVMLTNDNTSRY

SEQ ID NO: 145 - protein, 9 aa, Severe acute respiratory syndrome coronavirus 2
WTAGAAAYY

SEQ ID NO: 146 - protein, 9 aa, Severe acute respiratory syndrome coronavirus 2
TSNQVAVLY

SEQ ID NO: 147 - protein, 10 aa, Severe acute respiratory syndrome coronavirus 2
LADAGFIKQY

SEQ ID NO: 148 - protein, 14 aa, Severe acute respiratory syndrome coronavirus 2
VLPPLLTDEMIAQY

SEQ ID NO: 149 - protein, 10 aa, Severe acute respiratory syndrome coronavirus 2
FLVQAGNVQL

SEQ ID NO: 150 - protein, 9 aa, Severe acute respiratory syndrome coronavirus 2
FLNRFTTTL

SEQ ID NO: 151 - protein, 12 aa, Severe acute respiratory syndrome coronavirus 2
ALLEDEFTPFDV

SEQ ID NO: 152 - protein, 9 aa, Severe acute respiratory syndrome coronavirus 2
SLVKPSFYV

SEQ ID NO: 153 - protein, 9 aa, Severe acute respiratory syndrome coronavirus 2
KLSYGIATV

SEQ ID NO: 154 - protein, 9 aa, Severe acute respiratory syndrome coronavirus 2
TLVPQEHYV

SEQ ID NO: 155 - protein, 9 aa, Severe acute respiratory syndrome coronavirus 2
KLLEQWNLV

SEQ ID NO: 156 - protein, 9 aa, Severe acute respiratory syndrome coronavirus 2
GLVEVEKGV

SEQ ID NO: 157 - protein, 9 aa, Severe acute respiratory syndrome coronavirus 2
VMVELVAEL

SEQ ID NO: 158 - protein, 9 aa, Severe acute respiratory syndrome coronavirus 2
TLGVLVPHV

SEQ ID NO: 159 - protein, 9 aa, Severe acute respiratory syndrome coronavirus 2
YLASGGQPI

SEQ ID NO: 160 - protein, 9 aa, Severe acute respiratory syndrome coronavirus 2
MLSDTLKNL

SEQ ID NO: 161 - protein, 9 aa, Severe acute respiratory syndrome coronavirus 2
NLSDRVVFV

SEQ ID NO: 162 - protein, 9 aa, Severe acute respiratory syndrome coronavirus 2
VLWAHGFEL

SEQ ID NO: 163 - protein, 9 aa, Severe acute respiratory syndrome coronavirus 2
LLADKFPVL

SEQ ID NO: 164 - protein, 9 aa, Severe acute respiratory syndrome coronavirus 2
YLDAYNMMI

SEQ ID NO: 165 - protein, 9 aa, Severe acute respiratory syndrome coronavirus 2
MMISAGFSL

SEQ ID NO: 166 - protein, 9 aa, Severe acute respiratory syndrome coronavirus 2
SLENVAFNV

SEQ ID NO: 167 - protein, 9 aa, Severe acute respiratory syndrome coronavirus 2
SQLGGLHLL

SEQ ID NO: 168 - protein, 10 aa, Severe acute respiratory syndrome coronavirus 2
LLLDDFVEII

SEQ ID NO: 169 - protein, 9 aa, Severe acute respiratory syndrome coronavirus 2
YLNTLTLAV

SEQ ID NO: 170 - protein, 9 aa, Severe acute respiratory syndrome coronavirus 2
TLIGDCATV

SEQ ID NO: 171 - protein, 12 aa, Severe acute respiratory syndrome coronavirus 2
SLFDMSKFPLKL

SEQ ID NO: 172 - protein, 10 aa, Severe acute respiratory syndrome coronavirus 2
GLNDNLLEIL

SEQ ID NO: 173 - protein, 9 aa, Severe acute respiratory syndrome coronavirus 2
KLNEEIAII

SEQ ID NO: 174 - protein, 9 aa, Severe acute respiratory syndrome coronavirus 2
RLIDAMMFT

SEQ ID NO: 175 - protein, 9 aa, Severe acute respiratory syndrome coronavirus 2
YITGGVVQL

SEQ ID NO: 176 - protein, 9 aa, Severe acute respiratory syndrome coronavirus 2
KLVNKFLAL

SEQ ID NO: 177 - protein, 9 aa, Severe acute respiratory syndrome coronavirus 2
ALNLGETFV

SEQ ID NO: 178 - protein, 9 aa, Severe acute respiratory syndrome coronavirus 2
YLITPVHVM

SEQ ID NO: 179 - protein, 9 aa, Severe acute respiratory syndrome coronavirus 2
FLPRVFSAV

SEQ ID NO: 180 - protein, 9 aa, Severe acute respiratory syndrome coronavirus 2
KLIEYTDFA

SEQ ID NO: 181 - protein, 9 aa, Severe acute respiratory syndrome coronavirus 2
IVAGGIVAI

SEQ ID NO: 182 - protein, 9 aa, Severe acute respiratory syndrome coronavirus 2
FLAHIQWMV

SEQ ID NO: 183 - protein, 9 aa, Severe acute respiratory syndrome coronavirus 2
FLLNKEMYL

SEQ ID NO: 184 - protein, 9 aa, Severe acute respiratory syndrome coronavirus 2
ILTSLLVLV

SEQ ID NO: 185 - protein, 9 aa, Severe acute respiratory syndrome coronavirus 2
WLDMVDTSL

SEQ ID NO: 186 - protein, 9 aa, Severe acute respiratory syndrome coronavirus 2
TLMNVLTLV

SEQ ID NO: 187 - protein, 9 aa, Severe acute respiratory syndrome coronavirus 2
SMWALIISV

SEQ ID NO: 188 - protein, 9 aa, Severe acute respiratory syndrome coronavirus 2
VLLSVLQQL

SEQ ID NO: 189 - protein, 9 aa, Severe acute respiratory syndrome coronavirus 2
KLWAQCVQL

SEQ ID NO: 190 - protein, 9 aa, Severe acute respiratory syndrome coronavirus 2
ALWEIQQVV

SEQ ID NO: 191 - protein, 9 aa, Severe acute respiratory syndrome coronavirus 2
ALLSDLQDL

SEQ ID NO: 192 - protein, 9 aa, Severe acute respiratory syndrome coronavirus 2
LLLDRLNQL

SEQ ID NO: 193 - protein, 9 aa, Severe acute respiratory syndrome coronavirus 2
NVFAFPFTI

SEQ ID NO: 194 - protein, 9 aa, Severe acute respiratory syndrome coronavirus 2
ALSKGVHFV

SEQ ID NO: 195 - protein, 9 aa, Severe acute respiratory syndrome coronavirus 2
TVYSHLLLV

SEQ ID NO: 196 - protein, 9 aa, Severe acute respiratory syndrome coronavirus 2
YLYALVYFL

SEQ ID NO: 197 - protein, 9 aa, Severe acute respiratory syndrome coronavirus 2
LLYDANYFL

SEQ ID NO: 198 - protein, 9 aa, Severe acute respiratory syndrome coronavirus 2
HLVDFQVTI

SEQ ID NO: 199 - protein, 9 aa, Severe acute respiratory syndrome coronavirus 2
KLFIRQEEV

SEQ ID NO: 200 - protein, 11 aa, Severe acute respiratory syndrome coronavirus 2
YLFDESGEFKL

SEQ ID NO: 201 - protein, 11 aa, Severe acute respiratory syndrome coronavirus 2
YLKLTDNVYIK

SEQ ID NO: 202 - protein, 9 aa, Severe acute respiratory syndrome coronavirus 2
FLKKDAPYI

SEQ ID NO: 203 - protein, 9 aa, Severe acute respiratory syndrome coronavirus 2
TLNDLNETL

SEQ ID NO: 204 - protein, 10 aa, Severe acute respiratory syndrome coronavirus 2
YLDGADVTKI

SEQ ID NO: 205 - protein, 9 aa, Severe acute respiratory syndrome coronavirus 2
YLATALLTL

SEQ ID NO: 206 - protein, 9 aa, Severe acute respiratory syndrome coronavirus 2
YLNSTNVTI

SEQ ID NO: 207 - protein, 11 aa, Severe acute respiratory syndrome coronavirus 2
QMAPISAMVRM

SEQ ID NO: 208 - protein, 9 aa, Severe acute respiratory syndrome coronavirus 2
YVWKSYVHV

SEQ ID NO: 209 - protein, 9 aa, Severe acute respiratory syndrome coronavirus 2
ILLLDQALV

SEQ ID NO: 210 - protein, 9 aa, Severe acute respiratory syndrome coronavirus 2
NLIDSYFVV

SEQ ID NO: 211 - protein, 10 aa, Severe acute respiratory syndrome coronavirus 2
YTMADLVYAL

SEQ ID NO: 212 - protein, 9 aa, Severe acute respiratory syndrome coronavirus 2
SLLMPILTL

SEQ ID NO: 213 - protein, 10 aa, Severe acute respiratory syndrome coronavirus 2
KIFVDGVPFV

SEQ ID NO: 214 - protein, 9 aa, Severe acute respiratory syndrome coronavirus 2
RLANECAQV

SEQ ID NO: 215 - protein, 14 aa, Severe acute respiratory syndrome coronavirus 2
YLPYPDPSRILREF

SEQ ID NO: 216 - protein, 9 aa, Severe acute respiratory syndrome coronavirus 2
LMIERFVSL

SEQ ID NO: 217 - protein, 9 aa, Severe acute respiratory syndrome coronavirus 2
MLDMYSVML

SEQ ID NO: 218 - protein, 9 aa, Severe acute respiratory syndrome coronavirus 2
YLQPRTFLL

SEQ ID NO: 219 - protein, 9 aa, Severe acute respiratory syndrome coronavirus 2
KIADYNYKL

SEQ ID NO: 220 - protein, 9 aa, Severe acute respiratory syndrome coronavirus 2
SIIAYTMSL

SEQ ID NO: 221 - protein, 9 aa, Severe acute respiratory syndrome coronavirus 2
LLFNKVTLA

SEQ ID NO: 222 - protein, 9 aa, Severe acute respiratory syndrome coronavirus 2
VLNDILSRL

SEQ ID NO: 223 - protein, 9 aa, Severe acute respiratory syndrome coronavirus 2
RLDKVEAEV

SEQ ID NO: 224 - protein, 9 aa, Severe acute respiratory syndrome coronavirus 2
RLQSLQTYV

SEQ ID NO: 225 - protein, 9 aa, Severe acute respiratory syndrome coronavirus 2
HLMSFPQSA

SEQ ID NO: 226 - protein, 9 aa, Severe acute respiratory syndrome coronavirus 2
FIAGLIAIV

SEQ ID NO: 227 - protein, 9 aa, Severe acute respiratory syndrome coronavirus 2
AMRPNFTIK

SEQ ID NO: 228 - protein, 9 aa, Severe acute respiratory syndrome coronavirus 2
KLFAAETLK

SEQ ID NO: 229 - protein, 10 aa, Severe acute respiratory syndrome coronavirus 2
YVFTGYRVTK

SEQ ID NO: 230 - protein, 10 aa, Severe acute respiratory syndrome coronavirus 2
VVYRGTTTYK

SEQ ID NO: 231 - protein, 9 aa, Severe acute respiratory syndrome coronavirus 2
ALKYLPIDK

SEQ ID NO: 232 - protein, 9 aa, Severe acute respiratory syndrome coronavirus 2
VVNARLRAK

SEQ ID NO: 233 - protein, 9 aa, Severe acute respiratory syndrome coronavirus 2
RIAGHHLGR

SEQ ID NO: 234 - protein, 9 aa, Severe acute respiratory syndrome coronavirus 2
SLVPGFNEK

SEQ ID NO: 235 - protein, 9 aa, Severe acute respiratory syndrome coronavirus 2
RLISMMGFK

SEQ ID NO: 236 - protein, 9 aa, Severe acute respiratory syndrome coronavirus 2
SMMGFKMNY

SEQ ID NO: 237 - protein, 9 aa, Severe acute respiratory syndrome coronavirus 2
VLHDIGNPK

SEQ ID NO: 238 - protein, 10 aa, Severe acute respiratory syndrome coronavirus 2
SIINNTVYTK

SEQ ID NO: 239 - protein, 9 aa, Severe acute respiratory syndrome coronavirus 2
GLQPSVGPK

SEQ ID NO: 240 - protein, 10 aa, Severe acute respiratory syndrome coronavirus 2
GVAMPNLYKM

SEQ ID NO: 241 - protein, 9 aa, Severe acute respiratory syndrome coronavirus 2
KMQRMLLEK

SEQ ID NO: 242 - protein, 9 aa, Severe acute respiratory syndrome coronavirus 2
ALGGSVAIK

SEQ ID NO: 243 - protein, 10 aa, Severe acute respiratory syndrome coronavirus 2
KTIQPRVEKK

SEQ ID NO: 244 - protein, 9 aa, Severe acute respiratory syndrome coronavirus 2
KVTKGKAKK

SEQ ID NO: 245 - protein, 9 aa, Severe acute respiratory syndrome coronavirus 2
NIFGTVYEK

SEQ ID NO: 246 - protein, 9 aa, Severe acute respiratory syndrome coronavirus 2
IIIGGAKLK

SEQ ID NO: 247 - protein, 9 aa, Severe acute respiratory syndrome coronavirus 2
VTNNTFTLK

SEQ ID NO: 248 - protein, 9 aa, Severe acute respiratory syndrome coronavirus 2
TLKGGAPTK

SEQ ID NO: 249 - protein, 9 aa, Severe acute respiratory syndrome coronavirus 2
TIFKDASGK

SEQ ID NO: 250 - protein, 9 aa, Severe acute respiratory syndrome coronavirus 2
ALCTFLLNK

SEQ ID NO: 251 - protein, 10 aa, Severe acute respiratory syndrome coronavirus 2
SMQGAVDINK

SEQ ID NO: 252 - protein, 10 aa, Severe acute respiratory syndrome coronavirus 2
KLKKSLNVAK

SEQ ID NO: 253 - protein, 9 aa, Severe acute respiratory syndrome coronavirus 2
TMLFTMLRK

SEQ ID NO: 254 - protein, 10 aa, Severe acute respiratory syndrome coronavirus 2
VVIPDYNTYK

SEQ ID NO: 255 - protein, 9 aa, Severe acute respiratory syndrome coronavirus 2
ALRANSAVK

SEQ ID NO: 256 - protein, 9 aa, Severe acute respiratory syndrome coronavirus 2
ALAYYNTTK

SEQ ID NO: 257 - protein, 11 aa, Severe acute respiratory syndrome coronavirus 2
RIFTIGTVTLK

SEQ ID NO: 258 - protein, 9 aa, Severe acute respiratory syndrome coronavirus 2
SASKIITLK

SEQ ID NO: 259 - protein, 9 aa, Severe acute respiratory syndrome coronavirus 2
ASKIITLKK

SEQ ID NO: 260 - protein, 9 aa, Severe acute respiratory syndrome coronavirus 2
TVIEVQGYK

SEQ ID NO: 261 - protein, 9 aa, Severe acute respiratory syndrome coronavirus 2
RIDKVLNEK

SEQ ID NO: 262 - protein, 9 aa, Severe acute respiratory syndrome coronavirus 2
VLSGHNLAK

SEQ ID NO: 263 - protein, 9 aa, Severe acute respiratory syndrome coronavirus 2
HVVGPNVNK

SEQ ID NO: 264 - protein, 10 aa, Severe acute respiratory syndrome coronavirus 2
AVFDKNLYDK

SEQ ID NO: 265 - protein, 10 aa, Severe acute respiratory syndrome coronavirus 2
KLMPVCVETK

SEQ ID NO: 266 - protein, 9 aa, Severe acute respiratory syndrome coronavirus 2
AIVSTIQRK

SEQ ID NO: 267 - protein, 9 aa, Severe acute respiratory syndrome coronavirus 2
TISLAGSYK

SEQ ID NO: 268 - protein, 10 aa, Severe acute respiratory syndrome coronavirus 2
AVMYMGTLSY

SEQ ID NO: 269 - protein, 9 aa, Severe acute respiratory syndrome coronavirus 2
VVENPTIQK

SEQ ID NO: 270 - protein, 10 aa, Severe acute respiratory syndrome coronavirus 2
KASMPTTIAK

SEQ ID NO: 271 - protein, 9 aa, Severe acute respiratory syndrome coronavirus 2
ATAEAELAK

SEQ ID NO: 272 - protein, 10 aa, Severe acute respiratory syndrome coronavirus 2
RQVVNVVTTK

SEQ ID NO: 273 - protein, 9 aa, Severe acute respiratory syndrome coronavirus 2
VVTTKIALK

SEQ ID NO: 274 - protein, 10 aa, Severe acute respiratory syndrome coronavirus 2
KVAGFAKFLK

SEQ ID NO: 275 - protein, 10 aa, Severe acute respiratory syndrome coronavirus 2
LLKDCPAVAK

SEQ ID NO: 276 - protein, 9 aa, Severe acute respiratory syndrome coronavirus 2
AVAKHDFFK

SEQ ID NO: 277 - protein, 9 aa, Severe acute respiratory syndrome coronavirus 2
RVYANLGER

SEQ ID NO: 278 - protein, 9 aa, Severe acute respiratory syndrome coronavirus 2
KLFDRYFKY

SEQ ID NO: 279 - protein, 14 aa, Severe acute respiratory syndrome coronavirus 2
TVFPPTSFGPLVRK

SEQ ID NO: 280 - protein, 9 aa, Severe acute respiratory syndrome coronavirus 2
TSFGPLVRK

SEQ ID NO: 281 - protein, 9 aa, Severe acute respiratory syndrome coronavirus 2
TVKPGNFNK

SEQ ID NO: 282 - protein, 9 aa, Severe acute respiratory syndrome coronavirus 2
KSAGFPFNK

SEQ ID NO: 283 - protein, 9 aa, Severe acute respiratory syndrome coronavirus 2
RLYYDSMSY

SEQ ID NO: 284 - protein, 12 aa, Severe acute respiratory syndrome coronavirus 2
MTNRQFHQKLLK

SEQ ID NO: 285 - protein, 9 aa, Severe acute respiratory syndrome coronavirus 2
ATVVIGTSK

SEQ ID NO: 286 - protein, 9 aa, Severe acute respiratory syndrome coronavirus 2
LVASIKNFK

SEQ ID NO: 287 - protein, 9 aa, Severe acute respiratory syndrome coronavirus 2
AIDAYPLTK

SEQ ID NO: 288 - protein, 9 aa, Severe acute respiratory syndrome coronavirus 2
KVFRSSVLH

SEQ ID NO: 289 - protein, 9 aa, Severe acute respiratory syndrome coronavirus 2
GVYFASTEK

SEQ ID NO: 290 - protein, 9 aa, Severe acute respiratory syndrome coronavirus 2
GVYYHKNNK

SEQ ID NO: 291 - protein, 9 aa, Severe acute respiratory syndrome coronavirus 2
TLKSFTVEK

SEQ ID NO: 292 - protein, 10 aa, Severe acute respiratory syndrome coronavirus 2
RQIAPGQTGK

SEQ ID NO: 293 - protein, 9 aa, Severe acute respiratory syndrome coronavirus 2
RLFRKSNLK

SEQ ID NO: 294 - protein, 10 aa, Severe acute respiratory syndrome coronavirus 2
KQIYKTPPIK

SEQ ID NO: 295 - protein, 9 aa, Severe acute respiratory syndrome coronavirus 2
TLADAGFIK

SEQ ID NO: 296 - protein, 9 aa, Severe acute respiratory syndrome coronavirus 2
ASANLAATK

SEQ ID NO: 297 - protein, 9 aa, Severe acute respiratory syndrome coronavirus 2
VTYVPAQEK

SEQ ID NO: 298 - protein, 10 aa, Severe acute respiratory syndrome coronavirus 2
AVLDMCASLK

SEQ ID NO: 299 - protein, 9 aa, Severe acute respiratory syndrome coronavirus 2
VTFQSAVKR

SEQ ID NO: 300 - protein, 9 aa, Severe acute respiratory syndrome coronavirus 2
SSNVANYQK

SEQ ID NO: 301 - protein, 11 aa, Severe acute respiratory syndrome coronavirus 2
STLQGPPGTGK

SEQ ID NO: 302 - protein, 9 aa, Severe acute respiratory syndrome coronavirus 2
GTITVEELK

SEQ ID NO: 303 - protein, 10 aa, Severe acute respiratory syndrome coronavirus 2
ATSRTLSYYK

SEQ ID NO: 304 - protein, 9 aa, Severe acute respiratory syndrome coronavirus 2
ASCDAIMTR

SEQ ID NO: 305 - protein, 9 aa, Severe acute respiratory syndrome coronavirus 2
KTQFNYYKK

SEQ ID NO: 306 - protein, 9 aa, Severe acute respiratory syndrome coronavirus 2
HSWNADLYK

SEQ ID NO: 307 - protein, 10 aa, Severe acute respiratory syndrome coronavirus 2
SSYSLFDMSK

SEQ ID NO: 308 - protein, 10 aa, Severe acute respiratory syndrome coronavirus 2
STSAFVETVK

SEQ ID NO: 309 - protein, 9 aa, Severe acute respiratory syndrome coronavirus 2
RVVRSIFSR

SEQ ID NO: 310 - protein, 9 aa, Severe acute respiratory syndrome coronavirus 2
GAMDTTSYR

SEQ ID NO: 311 - protein, 10 aa, Severe acute respiratory syndrome coronavirus 2
SAVLQSGFRK

SEQ ID NO: 312 - protein, 9 aa, Severe acute respiratory syndrome coronavirus 2
SAFAMMFVK

SEQ ID NO: 313 - protein, 12 aa, Severe acute respiratory syndrome coronavirus 2
AVANGDSEVVLK

SEQ ID NO: 314 - protein, 9 aa, Severe acute respiratory syndrome coronavirus 2
QTMLFTMLR

SEQ ID NO: 315 - protein, 9 aa, Severe acute respiratory syndrome coronavirus 2
ASAFFGMSR

SEQ ID NO: 316 - protein, 11 aa, Severe acute respiratory syndrome coronavirus 2
AYKTFPPTEPK

SEQ ID NO: 317 - protein, 10 aa, Severe acute respiratory syndrome coronavirus 2
VVNAANVYLK

SEQ ID NO: 318 - protein, 11 aa, Severe acute respiratory syndrome coronavirus 2
LVSDIDITFLK

SEQ ID NO: 319 - protein, 9 aa, Severe acute respiratory syndrome coronavirus 2
LTAVVIPTK

SEQ ID NO: 320 - protein, 10 aa, Severe acute respiratory syndrome coronavirus 2
STQLGIEFLK

SEQ ID NO: 321 - protein, 9 aa, Severe acute respiratory syndrome coronavirus 2
GTLSYEQFK

SEQ ID NO: 322 - protein, 12 aa, Severe acute respiratory syndrome coronavirus 2
SYTTTIKPVTYK

SEQ ID NO: 323 - protein, 9 aa, Severe acute respiratory syndrome coronavirus 2
TSNSFDVLK

SEQ ID NO: 324 - protein, 9 aa, Severe acute respiratory syndrome coronavirus 2
TTIAKNTVK

SEQ ID NO: 325 - protein, 13 aa, Severe acute respiratory syndrome coronavirus 2
ASFNYLKSPNFSK

SEQ ID NO: 326 - protein, 12 aa, Severe acute respiratory syndrome coronavirus 2
SSTFNVPMEKLK

SEQ ID NO: 327 - protein, 9 aa, Severe acute respiratory syndrome coronavirus 2
GTSTDVVYR

SEQ ID NO: 328 - protein, 9 aa, Severe acute respiratory syndrome coronavirus 2
RAFDIYNDK

SEQ ID NO: 329 - protein, 9 aa, Severe acute respiratory syndrome coronavirus 2
NSASFSTFK

SEQ ID NO: 330 - protein, 10 aa, Severe acute respiratory syndrome coronavirus 2
TEILPVSMTK

SEQ ID NO: 331 - protein, 9 aa, Severe acute respiratory syndrome coronavirus 2
SSTASALGK

SEQ ID NO: 332 - protein, 9 aa, Severe acute respiratory syndrome coronavirus 2
GTHWFVTQR

SEQ ID NO: 333 - protein, 14 aa, Severe acute respiratory syndrome coronavirus 2
TVYDPLQPELDSFK

SEQ ID NO: 334 - protein, 13 aa, Severe acute respiratory syndrome coronavirus 2
KYKFVRIQPGQTF

SEQ ID NO: 335 - protein, 13 aa, Severe acute respiratory syndrome coronavirus 2
SYYCKSHKPPISF

SEQ ID NO: 336 - protein, 11 aa, Severe acute respiratory syndrome coronavirus 2
TYKLNVGDYFV

SEQ ID NO: 337 - protein, 9 aa, Severe acute respiratory syndrome coronavirus 2
VYIGDPAQL

SEQ ID NO: 338 - protein, 10 aa, Severe acute respiratory syndrome coronavirus 2
SYFIASFRLF

SEQ ID NO: 339 - protein, 9 aa, Severe acute respiratory syndrome coronavirus 2
SYGADLKSF

SEQ ID NO: 340 - protein, 9 aa, Severe acute respiratory syndrome coronavirus 2
VFVLWAHGF

SEQ ID NO: 341 - protein, 9 aa, Severe acute respiratory syndrome coronavirus 2
SYATHSDKF

SEQ ID NO: 342 - protein, 9 aa, Severe acute respiratory syndrome coronavirus 2
KFTDGVCLF

SEQ ID NO: 343 - protein, 9 aa, Severe acute respiratory syndrome coronavirus 2
LYLDAYNMM

SEQ ID NO: 344 - protein, 11 aa, Severe acute respiratory syndrome coronavirus 2
VYKQFDTYNLW

SEQ ID NO: 345 - protein, 14 aa, Severe acute respiratory syndrome coronavirus 2
VFFDGRVDGQVDLF

SEQ ID NO: 346 - protein, 9 aa, Severe acute respiratory syndrome coronavirus 2
RYKLEGYAF

SEQ ID NO: 347 - protein, 9 aa, Severe acute respiratory syndrome coronavirus 2
TFFKLVNKF

SEQ ID NO: 348 - protein, 9 aa, Severe acute respiratory syndrome coronavirus 2
VFLFVAAIF

SEQ ID NO: 349 - protein, 13 aa, Severe acute respiratory syndrome coronavirus 2
TYLEGSVRVVTTF

SEQ ID NO: 350 - protein, 9 aa, Severe acute respiratory syndrome coronavirus 2
YYRSLPGVF

SEQ ID NO: 351 - protein, 10 aa, Severe acute respiratory syndrome coronavirus 2
GVYSVIYLYL

SEQ ID NO: 352 - protein, 11 aa, Severe acute respiratory syndrome coronavirus 2
TFYLTNDVSFL

SEQ ID NO: 353 - protein, 9 aa, Severe acute respiratory syndrome coronavirus 2
MFTPLVPFW

SEQ ID NO: 354 - protein, 10 aa, Severe acute respiratory syndrome coronavirus 2
VFNGVSFSTF

SEQ ID NO: 355 - protein, 9 aa, Severe acute respiratory syndrome coronavirus 2
LYQPPQTSI

SEQ ID NO: 356 - protein, 14 aa, Severe acute respiratory syndrome coronavirus 2
VYMPASWVMRIMTW

SEQ ID NO: 357 - protein, 9 aa, Severe acute respiratory syndrome coronavirus 2
MYASAVVLL

SEQ ID NO: 358 - protein, 9 aa, Severe acute respiratory syndrome coronavirus 2
MFLARGIVF

SEQ ID NO: 359 - protein, 9 aa, Severe acute respiratory syndrome coronavirus 2
IFFITGNTL

SEQ ID NO: 360 - protein, 10 aa, Severe acute respiratory syndrome coronavirus 2
YDYLVSTQEF

SEQ ID NO: 361 - protein, 9 aa, Severe acute respiratory syndrome coronavirus 2
TYASALWEI

SEQ ID NO: 362 - protein, 9 aa, Severe acute respiratory syndrome coronavirus 2
YFIKGLNNL

SEQ ID NO: 363 - protein, 10 aa, Severe acute respiratory syndrome coronavirus 2
GYINVFAFPF

SEQ ID NO: 364 - protein, 9 aa, Severe acute respiratory syndrome coronavirus 2
LYLYALVYF

SEQ ID NO: 365 - protein, 9 aa, Severe acute respiratory syndrome coronavirus 2
VYFLQSINF

SEQ ID NO: 366 - protein, 9 aa, Severe acute respiratory syndrome coronavirus 2
YYQLYSTQL

SEQ ID NO: 367 - protein, 10 aa, Severe acute respiratory syndrome coronavirus 2
QYIDIGNYTV

SEQ ID NO: 368 - protein, 10 aa, Severe acute respiratory syndrome coronavirus 2
QGYKSVNITF

SEQ ID NO: 369 - protein, 9 aa, Severe acute respiratory syndrome coronavirus 2
DYQGKPLEF

SEQ ID NO: 370 - protein, 9 aa, Severe acute respiratory syndrome coronavirus 2
LYDKLVSSF

SEQ ID NO: 371 - protein, 9 aa, Severe acute respiratory syndrome coronavirus 2
LYIDINGNL

SEQ ID NO: 372 - protein, 10 aa, Severe acute respiratory syndrome coronavirus 2
VYYTSNPTTF

SEQ ID NO: 373 - protein, 10 aa, Severe acute respiratory syndrome coronavirus 2
EYYHTTDPSF

SEQ ID NO: 374 - protein, 9 aa, Severe acute respiratory syndrome coronavirus 2
SYLFQHANL

SEQ ID NO: 375 - protein, 10 aa, Severe acute respiratory syndrome coronavirus 2
NYYKKDNSYF

SEQ ID NO: 376 - protein, 9 aa, Severe acute respiratory syndrome coronavirus 2
DYKHYTPSF

SEQ ID NO: 377 - protein, 9 aa, Severe acute respiratory syndrome coronavirus 2
NYMPYFFTL

SEQ ID NO: 378 - protein, 11 aa, Severe acute respiratory syndrome coronavirus 2
AYILFTRFFYV

SEQ ID NO: 379 - protein, 9 aa, Severe acute respiratory syndrome coronavirus 2
FFASFYYVW

SEQ ID NO: 380 - protein, 9 aa, Severe acute respiratory syndrome coronavirus 2
VYANGGKGF

SEQ ID NO: 381 - protein, 10 aa, Severe acute respiratory syndrome coronavirus 2
KMFDAYVNTF

SEQ ID NO: 382 - protein, 10 aa, Severe acute respiratory syndrome coronavirus 2
AYVNTFSSTF

SEQ ID NO: 383 - protein, 12 aa, Severe acute respiratory syndrome coronavirus 2
IYNDKVAGFAKF

SEQ ID NO: 384 - protein, 10 aa, Severe acute respiratory syndrome coronavirus 2
SYFVVKRHTF

SEQ ID NO: 385 - protein, 9 aa, Severe acute respiratory syndrome coronavirus 2
NFNKDFYDF

SEQ ID NO: 386 - protein, 9 aa, Severe acute respiratory syndrome coronavirus 2
AYANSVFNI

SEQ ID NO: 387 - protein, 10 aa, Severe acute respiratory syndrome coronavirus 2
HLYLQYIRKL

SEQ ID NO: 388 - protein, 9 aa, Severe acute respiratory syndrome coronavirus 2
PFFSNVTWF

SEQ ID NO: 389 - protein, 9 aa, Severe acute respiratory syndrome coronavirus 2
RFDNPVLPF

SEQ ID NO: 390 - protein, 10 aa, Severe acute respiratory syndrome coronavirus 2
VYSSANNCTF

SEQ ID NO: 391 - protein, 9 aa, Severe acute respiratory syndrome coronavirus 2
EYVSQPFLM

SEQ ID NO: 392 - protein, 11 aa, Severe acute respiratory syndrome coronavirus 2
YYVGYLQPRTF

SEQ ID NO: 393 - protein, 10 aa, Severe acute respiratory syndrome coronavirus 2
LYNSASFSTF

SEQ ID NO: 394 - protein, 9 aa, Severe acute respiratory syndrome coronavirus 2
NYNYLYRLF

SEQ ID NO: 395 - protein, 9 aa, Severe acute respiratory syndrome coronavirus 2
YFPLQSYGF

SEQ ID NO: 396 - protein, 9 aa, Severe acute respiratory syndrome coronavirus 2
PYRVVVLSF

SEQ ID NO: 397 - protein, 9 aa, Severe acute respiratory syndrome coronavirus 2
VYSTGSNVF

SEQ ID NO: 398 - protein, 10 aa, Severe acute respiratory syndrome coronavirus 2
IYKTPPIKDF

SEQ ID NO: 399 - protein, 10 aa, Severe acute respiratory syndrome coronavirus 2
TYVPAQEKNF

SEQ ID NO: 400 - protein, 9 aa, Severe acute respiratory syndrome coronavirus 2
QYIKWPWYI

SEQ ID NO: 401 - protein, 9 aa, Severe acute respiratory syndrome coronavirus 2
DYVYNPFMI

SEQ ID NO: 402 - protein, 8 aa, Severe acute respiratory syndrome coronavirus 2
TYNLWNTF

SEQ ID NO: 403 - protein, 9 aa, Severe acute respiratory syndrome coronavirus 2
NYLKRRVVF

SEQ ID NO: 404 - protein, 11 aa, Severe acute respiratory syndrome coronavirus 2
NYSGVVTTVMF

SEQ ID NO: 405 - protein, 9 aa, Severe acute respiratory syndrome coronavirus 2
AFPFTIYSL

SEQ ID NO: 406 - protein, 9 aa, Severe acute respiratory syndrome coronavirus 2
PYNSVTSSI

SEQ ID NO: 407 - protein, 12 aa, Severe acute respiratory syndrome coronavirus 2
YYRARAGEAANF

SEQ ID NO: 408 - protein, 11 aa, Severe acute respiratory syndrome coronavirus 2
MYMGTLSYEQF

SEQ ID NO: 409 - protein, 12 aa, Severe acute respiratory syndrome coronavirus 2
PYPNASFDNFKF

SEQ ID NO: 410 - protein, 9 aa, Severe acute respiratory syndrome coronavirus 2
TYKPNTWCI

SEQ ID NO: 411 - protein, 9 aa, Severe acute respiratory syndrome coronavirus 2
FYGGWHNML

SEQ ID NO: 412 - protein, 9 aa, Severe acute respiratory syndrome coronavirus 2
YYHKNNKSW

SEQ ID NO: 413 - protein, 11 aa, Severe acute respiratory syndrome coronavirus 2
RFPNITNLCPF

SEQ ID NO: 414 - protein, 13 aa, Severe acute respiratory syndrome coronavirus 2
AYSNNSIAIPTNF

SEQ ID NO: 415 - protein, 9 aa, Severe acute respiratory syndrome coronavirus 2
QPGQTFSVL

SEQ ID NO: 416 - protein, 10 aa, Severe acute respiratory syndrome coronavirus 2
SPSGVYQCAM

SEQ ID NO: 417 - protein, 10 aa, Severe acute respiratory syndrome coronavirus 2
RPNFTIKGSF

SEQ ID NO: 418 - protein, 9 aa, Severe acute respiratory syndrome coronavirus 2
TPHTVLQAV

SEQ ID NO: 419 - protein, 12 aa, Severe acute respiratory syndrome coronavirus 2
KPRPPLNRNYVF

SEQ ID NO: 420 - protein, 8 aa, Severe acute respiratory syndrome coronavirus 2
MPLSAPTL

SEQ ID NO: 421 - protein, 10 aa, Severe acute respiratory syndrome coronavirus 2
IPARARVECF

SEQ ID NO: 422 - protein, 11 aa, Severe acute respiratory syndrome coronavirus 2
APRTLLTKGTL

SEQ ID NO: 423 - protein, 10 aa, Severe acute respiratory syndrome coronavirus 2
RPQIGVVREF

SEQ ID NO: 424 - protein, 10 aa, Severe acute respiratory syndrome coronavirus 2
RNPAWRKAVF

SEQ ID NO: 425 - protein, 14 aa, Severe acute respiratory syndrome coronavirus 2
SPYNSQNAVASKIL

SEQ ID NO: 426 - protein, 9 aa, Severe acute respiratory syndrome coronavirus 2
IPRRNVATL

SEQ ID NO: 427 - protein, 8 aa, Severe acute respiratory syndrome coronavirus 2
VPLHGTIL

SEQ ID NO: 428 - protein, 9 aa, Severe acute respiratory syndrome coronavirus 2
RTAPHGHVM

SEQ ID NO: 429 - protein, 10 aa, Severe acute respiratory syndrome coronavirus 2
APHGHVMVEL

SEQ ID NO: 430 - protein, 10 aa, Severe acute respiratory syndrome coronavirus 2
VPANSTVLSF

SEQ ID NO: 431 - protein, 10 aa, Severe acute respiratory syndrome coronavirus 2
HPNPKGFCDL

SEQ ID NO: 432 - protein, 9 aa, Severe acute respiratory syndrome coronavirus 2
HPTQAPTHL

SEQ ID NO: 433 - protein, 9 aa, Severe acute respiratory syndrome coronavirus 2
TPAFDKSAF

SEQ ID NO: 434 - protein, 9 aa, Severe acute respiratory syndrome coronavirus 2
LPVNVAFEL

SEQ ID NO: 435 - protein, 9 aa, Severe acute respiratory syndrome coronavirus 2
KPVPEVKIL

SEQ ID NO: 436 - protein, 8 aa, Severe acute respiratory syndrome coronavirus 2
APAHISTI

SEQ ID NO: 437 - protein, 12 aa, Severe acute respiratory syndrome coronavirus 2
GPKQASLNGVTL

SEQ ID NO: 438 - protein, 8 aa, Severe acute respiratory syndrome coronavirus 2
KPRSQMEI

SEQ ID NO: 439 - protein, 9 aa, Severe acute respiratory syndrome coronavirus 2
LPKGIMMNV

SEQ ID NO: 440 - protein, 10 aa, Severe acute respiratory syndrome coronavirus 2
VPYNMRVIHF

SEQ ID NO: 441 - protein, 11 aa, Severe acute respiratory syndrome coronavirus 2
KPREQIDGYVM

SEQ ID NO: 442 - protein, 9 aa, Severe acute respiratory syndrome coronavirus 2
QPRVEKKKL

SEQ ID NO: 443 - protein, 9 aa, Severe acute respiratory syndrome coronavirus 2
MPLKAPKEI

SEQ ID NO: 444 - protein, 8 aa, Severe acute respiratory syndrome coronavirus 2
APKEIIFL

SEQ ID NO: 445 - protein, 10 aa, Severe acute respiratory syndrome coronavirus 2
VVPGLPGTIL

SEQ ID NO: 446 - protein, 10 aa, Severe acute respiratory syndrome coronavirus 2
VPYCYDTNVL

SEQ ID NO: 447 - protein, 9 aa, Severe acute respiratory syndrome coronavirus 2
RPDTRYVLM

SEQ ID NO: 448 - protein, 10 aa, Severe acute respiratory syndrome coronavirus 2
TPLIQPIGAL

SEQ ID NO: 449 - protein, 10 aa, Severe acute respiratory syndrome coronavirus 2
LPFAMGIIAM

SEQ ID NO: 450 - protein, 9 aa, Severe acute respiratory syndrome coronavirus 2
IPLTTAAKL

SEQ ID NO: 451 - protein, 9 aa, Severe acute respiratory syndrome coronavirus 2
SPNLAWPLI

SEQ ID NO: 452 - protein, 8 aa, Severe acute respiratory syndrome coronavirus 2
SPVALRQM

SEQ ID NO: 453 - protein, 10 aa, Severe acute respiratory syndrome coronavirus 2
TPKGPKVKYL

SEQ ID NO: 454 - protein, 10 aa, Severe acute respiratory syndrome coronavirus 2
KFPRGQGVPI

SEQ ID NO: 455 - protein, 9 aa, Severe acute respiratory syndrome coronavirus 2
SPRWYFYYL

SEQ ID NO: 456 - protein, 9 aa, Severe acute respiratory syndrome coronavirus 2
IPIQASLPF

SEQ ID NO: 457 - protein, 9 aa, Severe acute respiratory syndrome coronavirus 2
APFLYLYAL

SEQ ID NO: 458 - protein, 13 aa, Severe acute respiratory syndrome coronavirus 2
SPFHPLADNKFAL

SEQ ID NO: 459 - protein, 9 aa, Severe acute respiratory syndrome coronavirus 2
RARSVSPKL

SEQ ID NO: 460 - protein, 12 aa, Severe acute respiratory syndrome coronavirus 2
KPLEFGATSAAL

SEQ ID NO: 461 - protein, 9 aa, Severe acute respiratory syndrome coronavirus 2
APLLSAGIF

SEQ ID NO: 462 - protein, 9 aa, Severe acute respiratory syndrome coronavirus 2
APYIVGDVV

SEQ ID NO: 463 - protein, 10 aa, Severe acute respiratory syndrome coronavirus 2
YPQVNGLTSI

SEQ ID NO: 464 - protein, 11 aa, Severe acute respiratory syndrome coronavirus 2
KPASRELKVTF

SEQ ID NO: 465 - protein, 10 aa, Severe acute respiratory syndrome coronavirus 2
TPSFKKGAKL

SEQ ID NO: 466 - protein, 9 aa, Severe acute respiratory syndrome coronavirus 2
KPANNSLKI

SEQ ID NO: 467 - protein, 10 aa, Severe acute respiratory syndrome coronavirus 2
KKPNELSRVL

SEQ ID NO: 468 - protein, 9 aa, Severe acute respiratory syndrome coronavirus 2
MPYFFTLLL

SEQ ID NO: 469 - protein, 9 aa, Severe acute respiratory syndrome coronavirus 2
TPRDLGACI

SEQ ID NO: 470 - protein, 9 aa, Severe acute respiratory syndrome coronavirus 2
VAKSHNIAL

SEQ ID NO: 471 - protein, 10 aa, Severe acute respiratory syndrome coronavirus 2
MVPHISRQRL

SEQ ID NO: 472 - protein, 10 aa, Severe acute respiratory syndrome coronavirus 2
MPILTLTRAL

SEQ ID NO: 473 - protein, 9 aa, Severe acute respiratory syndrome coronavirus 2
KPYIKWDLL

SEQ ID NO: 474 - protein, 9 aa, Severe acute respiratory syndrome coronavirus 2
IPTITQMNL

SEQ ID NO: 475 - protein, 9 aa, Severe acute respiratory syndrome coronavirus 2
LPFNDGVYF

SEQ ID NO: 476 - protein, 9 aa, Severe acute respiratory syndrome coronavirus 2
TPINLVRDL

SEQ ID NO: 477 - protein, 8 aa, Severe acute respiratory syndrome coronavirus 2
LPQGFSAL

SEQ ID NO: 478 - protein, 11 aa, Severe acute respiratory syndrome coronavirus 2
KPFERDISTEI

SEQ ID NO: 479 - protein, 8 aa, Severe acute respiratory syndrome coronavirus 2
QPYRVVVL

SEQ ID NO: 480 - protein, 9 aa, Severe acute respiratory syndrome coronavirus 2
GPKKSTNLV

SEQ ID NO: 481 - protein, 9 aa, Severe acute respiratory syndrome coronavirus 2
SPRRARSVA

SEQ ID NO: 482 - protein, 9 aa, Severe acute respiratory syndrome coronavirus 2
IPTNFTISV

SEQ ID NO: 483 - protein, 8 aa, Severe acute respiratory syndrome coronavirus 2
APHGVVFL

SEQ ID NO: 484 - protein, 10 aa, Severe acute respiratory syndrome coronavirus 2
SEPVLKGVKL

SEQ ID NO: 485 - protein, 9 aa, Severe acute respiratory syndrome coronavirus 2
QLRVIGHSM

SEQ ID NO: 486 - protein, 9 aa, Severe acute respiratory syndrome coronavirus 2
TPKYKFVRI

SEQ ID NO: 487 - protein, 9 aa, Severe acute respiratory syndrome coronavirus 2
YVYSRVKNL

SEQ ID NO: 488 - protein, 8 aa, Severe acute respiratory syndrome coronavirus 2
VGKPRPPL

SEQ ID NO: 489 - protein, 9 aa, Severe acute respiratory syndrome coronavirus 2
VGMQKYSTL

SEQ ID NO: 490 - protein, 9 aa, Severe acute respiratory syndrome coronavirus 2
YDKLQFTSL

SEQ ID NO: 491 - protein, 9 aa, Severe acute respiratory syndrome coronavirus 2
RLFARTRSM

SEQ ID NO: 492 - protein, 9 aa, Severe acute respiratory syndrome coronavirus 2
HLRIAGHHL

SEQ ID NO: 493 - protein, 9 aa, Severe acute respiratory syndrome coronavirus 2
DLKGKYVQI

SEQ ID NO: 494 - protein, 9 aa, Severe acute respiratory syndrome coronavirus 2
MTYRRLISM

SEQ ID NO: 495 - protein, 9 aa, Severe acute respiratory syndrome coronavirus 2
VVRIKIVQM

SEQ ID NO: 496 - protein, 8 aa, Severe acute respiratory syndrome coronavirus 2
HMVVKAAL

SEQ ID NO: 497 - protein, 9 aa, Severe acute respiratory syndrome coronavirus 2
SLYVNKHAF

SEQ ID NO: 498 - protein, 9 aa, Severe acute respiratory syndrome coronavirus 2
HANEYRLYL

SEQ ID NO: 499 - protein, 9 aa, Severe acute respiratory syndrome coronavirus 2
NTFTRLQSL

SEQ ID NO: 500 - protein, 9 aa, Severe acute respiratory syndrome coronavirus 2
SVGPKQASL

SEQ ID NO: 501 - protein, 8 aa, Severe acute respiratory syndrome coronavirus 2
NVAKYTQL

SEQ ID NO: 502 - protein, 8 aa, Severe acute respiratory syndrome coronavirus 2
FIQQKLAL

SEQ ID NO: 503 - protein, 9 aa, Severe acute respiratory syndrome coronavirus 2
GAKLKALNL

SEQ ID NO: 504 - protein, 9 aa, Severe acute respiratory syndrome coronavirus 2
HVMSKHTDF

SEQ ID NO: 505 - protein, 8 aa, Severe acute respiratory syndrome coronavirus 2
FMRFRRAF

SEQ ID NO: 506 - protein, 9 aa, Severe acute respiratory syndrome coronavirus 2
YLKLRSDVL

SEQ ID NO: 507 - protein, 9 aa, Severe acute respiratory syndrome coronavirus 2
TQYNRYLAL

SEQ ID NO: 508 - protein, 9 aa, Severe acute respiratory syndrome coronavirus 2
FVKHKHAFL

SEQ ID NO: 509 - protein, 9 aa, Severe acute respiratory syndrome coronavirus 2
DGARRVWTL

SEQ ID NO: 510 - protein, 9 aa, Severe acute respiratory syndrome coronavirus 2
EMLDNRATL

SEQ ID NO: 511 - protein, 9 aa, Severe acute respiratory syndrome coronavirus 2
VLKKLKKSL

SEQ ID NO: 512 - protein, 9 aa, Severe acute respiratory syndrome coronavirus 2
GPKVKYLYF

SEQ ID NO: 513 - protein, 9 aa, Severe acute respiratory syndrome coronavirus 2
NNLNRGMVL

SEQ ID NO: 514 - protein, 9 aa, Severe acute respiratory syndrome coronavirus 2
ITLKKRWQL

SEQ ID NO: 515 - protein, 9 aa, Severe acute respiratory syndrome coronavirus 2
IIKNLSKSL

SEQ ID NO: 516 - protein, 9 aa, Severe acute respiratory syndrome coronavirus 2
DGVKHVYQL

SEQ ID NO: 517 - protein, 8 aa, Severe acute respiratory syndrome coronavirus 2
NLKTLLSL

SEQ ID NO: 518 - protein, 9 aa, Severe acute respiratory syndrome coronavirus 2
TIKPVTYKL

SEQ ID NO: 519 - protein, 9 aa, Severe acute respiratory syndrome coronavirus 2
ILKPANNSL

SEQ ID NO: 520 - protein, 8 aa, Severe acute respiratory syndrome coronavirus 2
TIKKPNEL

SEQ ID NO: 521 - protein, 8 aa, Severe acute respiratory syndrome coronavirus 2
NSRIKASM

SEQ ID NO: 522 - protein, 8 aa, Severe acute respiratory syndrome coronavirus 2
KTYERHSL

SEQ ID NO: 523 - protein, 10 aa, Severe acute respiratory syndrome coronavirus 2
NVPMEKLKTL

SEQ ID NO: 524 - protein, 8 aa, Severe acute respiratory syndrome coronavirus 2
NVKDFMSL

SEQ ID NO: 525 - protein, 8 aa, Severe acute respiratory syndrome coronavirus 2
SAAKKNNL

SEQ ID NO: 526 - protein, 9 aa, Severe acute respiratory syndrome coronavirus 2
FAYTKRNVI

SEQ ID NO: 527 - protein, 9 aa, Severe acute respiratory syndrome coronavirus 2
SAKNRARTV

SEQ ID NO: 528 - protein, 9 aa, Severe acute respiratory syndrome coronavirus 2
IAATRGATV

SEQ ID NO: 529 - protein, 9 aa, Severe acute respiratory syndrome coronavirus 2
SLSHRFYRL

SEQ ID NO: 530 - protein, 8 aa, Severe acute respiratory syndrome coronavirus 2
YAYLRKHF

SEQ ID NO: 531 - protein, 9 aa, Severe acute respiratory syndrome coronavirus 2
YLRKHFSMM

SEQ ID NO: 532 - protein, 9 aa, Severe acute respiratory syndrome coronavirus 2
SIKNFKSVL

SEQ ID NO: 533 - protein, 8 aa, Severe acute respiratory syndrome coronavirus 2
NLTTRTQL

SEQ ID NO: 534 - protein, 9 aa, Severe acute respiratory syndrome coronavirus 2
TLDSKTQSL

SEQ ID NO: 535 - protein, 9 aa, Severe acute respiratory syndrome coronavirus 2
INITRFQTL

SEQ ID NO: 536 - protein, 9 aa, Severe acute respiratory syndrome coronavirus 2
LLALHRSYL

SEQ ID NO: 537 - protein, 8 aa, Severe acute respiratory syndrome coronavirus 2
YLQPRTFL

SEQ ID NO: 538 - protein, 9 aa, Severe acute respiratory syndrome coronavirus 2
LITGRLQSL

SEQ ID NO: 539 - protein, 9 aa, Severe acute respiratory syndrome coronavirus 2
TANPKTPKY

SEQ ID NO: 540 - protein, 10 aa, Severe acute respiratory syndrome coronavirus 2
FPLCANGQVF

SEQ ID NO: 541 - protein, 10 aa, Severe acute respiratory syndrome coronavirus 2
YPTLNISDEF

SEQ ID NO: 542 - protein, 9 aa, Severe acute respiratory syndrome coronavirus 2
FAIGLALYY

SEQ ID NO: 543 - protein, 8 aa, Severe acute respiratory syndrome coronavirus 2
YPSARIVY

SEQ ID NO: 544 - protein, 9 aa, Severe acute respiratory syndrome coronavirus 2
FAYANRNRF

SEQ ID NO: 545 - protein, 9 aa, Severe acute respiratory syndrome coronavirus 2
YANRNRFLY

SEQ ID NO: 546 - protein, 11 aa, Severe acute respiratory syndrome coronavirus 2
VPHVGEIPVAY

SEQ ID NO: 547 - protein, 9 aa, Severe acute respiratory syndrome coronavirus 2
HVGEIPVAY

SEQ ID NO: 548 - protein, 10 aa, Severe acute respiratory syndrome coronavirus 2
FAVDAAKAYK

SEQ ID NO: 549 - protein, 9 aa, Severe acute respiratory syndrome coronavirus 2
EANMDQESF

SEQ ID NO: 550 - protein, 10 aa, Severe acute respiratory syndrome coronavirus 2
IPGIPKDMTY

SEQ ID NO: 551 - protein, 9 aa, Severe acute respiratory syndrome coronavirus 2
HSIGFDYVY

SEQ ID NO: 552 - protein, 10 aa, Severe acute respiratory syndrome coronavirus 2
YPANSIVCRF

SEQ ID NO: 553 - protein, 9 aa, Severe acute respiratory syndrome coronavirus 2
EAVKTQFNY

SEQ ID NO: 554 - protein, 8 aa, Severe acute respiratory syndrome coronavirus 2
YAFEHIVY

SEQ ID NO: 555 - protein, 10 aa, Severe acute respiratory syndrome coronavirus 2
IPMDSTVKNY

SEQ ID NO: 556 - protein, 9 aa, Severe acute respiratory syndrome coronavirus 2
QAWQPGVAM

SEQ ID NO: 557 - protein, 9 aa, Severe acute respiratory syndrome coronavirus 2
TILDGISQY

SEQ ID NO: 558 - protein, 9 aa, Severe acute respiratory syndrome coronavirus 2
DASGKPVPY

SEQ ID NO: 559 - protein, 9 aa, Severe acute respiratory syndrome coronavirus 2
NVLEGSVAY

SEQ ID NO: 560 - protein, 9 aa, Severe acute respiratory syndrome coronavirus 2
SIIQFPNTY

SEQ ID NO: 561 - protein, 10 aa, Severe acute respiratory syndrome coronavirus 2
FLPGVYSVIY

SEQ ID NO: 562 - protein, 9 aa, Severe acute respiratory syndrome coronavirus 2
VPFWITIAY

SEQ ID NO: 563 - protein, 9 aa, Severe acute respiratory syndrome coronavirus 2
LPLTQYNRY

SEQ ID NO: 564 - protein, 11 aa, Severe acute respiratory syndrome coronavirus 2
FLLPSLATVAY

SEQ ID NO: 565 - protein, 9 aa, Severe acute respiratory syndrome coronavirus 2
NALDQAISM

SEQ ID NO: 566 - protein, 9 aa, Severe acute respiratory syndrome coronavirus 2
LAKDTTEAF

SEQ ID NO: 567 - protein, 9 aa, Severe acute respiratory syndrome coronavirus 2
LPNNTASWF

SEQ ID NO: 568 - protein, 9 aa, Severe acute respiratory syndrome coronavirus 2
KAYNVTQAF

SEQ ID NO: 569 - protein, 9 aa, Severe acute respiratory syndrome coronavirus 2
TPSGTWLTY

SEQ ID NO: 570 - protein, 9 aa, Severe acute respiratory syndrome coronavirus 2
LPAADLDDF

SEQ ID NO: 571 - protein, 9 aa, Severe acute respiratory syndrome coronavirus 2
NPLLYDANY

SEQ ID NO: 572 - protein, 13 aa, Severe acute respiratory syndrome coronavirus 2
FAFACPDGVKHVY

SEQ ID NO: 573 - protein, 9 aa, Severe acute respiratory syndrome coronavirus 2
YPGQGLNGY

SEQ ID NO: 574 - protein, 11 aa, Severe acute respiratory syndrome coronavirus 2
SPDAVTAYNGY

SEQ ID NO: 575 - protein, 9 aa, Severe acute respiratory syndrome coronavirus 2
QVVDMSMTY

SEQ ID NO: 576 - protein, 14 aa, Severe acute respiratory syndrome coronavirus 2
LPNDDTLRVEAFEY

SEQ ID NO: 577 - protein, 9 aa, Severe acute respiratory syndrome coronavirus 2
NPPALQDAY

SEQ ID NO: 578 - protein, 10 aa, Severe acute respiratory syndrome coronavirus 2
TKPVETSNSF

SEQ ID NO: 579 - protein, 9 aa, Severe acute respiratory syndrome coronavirus 2
VPWDTIANY

SEQ ID NO: 580 - protein, 9 aa, Severe acute respiratory syndrome coronavirus 2
YPSLETIQI

SEQ ID NO: 581 - protein, 9 aa, Severe acute respiratory syndrome coronavirus 2
VARDLSLQF

SEQ ID NO: 582 - protein, 9 aa, Severe acute respiratory syndrome coronavirus 2
SAKSASVYY

SEQ ID NO: 583 - protein, 9 aa, Severe acute respiratory syndrome coronavirus 2
VPFVVSTGY

SEQ ID NO: 584 - protein, 9 aa, Severe acute respiratory syndrome coronavirus 2
NAAISDYDY

SEQ ID NO: 585 - protein, 9 aa, Severe acute respiratory syndrome coronavirus 2
FVVEVVDKY

SEQ ID NO: 586 - protein, 9 aa, Severe acute respiratory syndrome coronavirus 2
NPHLMGWDY

SEQ ID NO: 587 - protein, 9 aa, Severe acute respiratory syndrome coronavirus 2
FVSLAIDAY

SEQ ID NO: 588 - protein, 11 aa, Severe acute respiratory syndrome coronavirus 2
YPLTKHPNQEY

SEQ ID NO: 589 - protein, 13 aa, Severe acute respiratory syndrome coronavirus 2
HPNQEYADVFHLY

SEQ ID NO: 590 - protein, 9 aa, Severe acute respiratory syndrome coronavirus 2
LPPAYTNSF

SEQ ID NO: 591 - protein, 9 aa, Severe acute respiratory syndrome coronavirus 2
LPFFSNVTW

SEQ ID NO: 592 - protein, 9 aa, Severe acute respiratory syndrome coronavirus 2
FVFKNIDGY

SEQ ID NO: 593 - protein, 10 aa, Severe acute respiratory syndrome coronavirus 2
LPIGINITRF

SEQ ID NO: 594 - protein, 9 aa, Severe acute respiratory syndrome coronavirus 2
QPTESIVRF

SEQ ID NO: 595 - protein, 9 aa, Severe acute respiratory syndrome coronavirus 2
NATRFASVY

SEQ ID NO: 596 - protein, 11 aa, Severe acute respiratory syndrome coronavirus 2
IPIGAGICASY

SEQ ID NO: 597 - protein, 9 aa, Severe acute respiratory syndrome coronavirus 2
VASQSIIAY

SEQ ID NO: 598 - protein, 9 aa, Severe acute respiratory syndrome coronavirus 2
LGAENSVAY

SEQ ID NO: 599 - protein, 9 aa, Severe acute respiratory syndrome coronavirus 2
IPFAMQMAY

SEQ ID NO: 600 - protein, 11 aa, Severe acute respiratory syndrome coronavirus 2
FPQSAPHGVVF

SEQ ID NO: 601 - protein, 11 aa, Severe acute respiratory syndrome coronavirus 2
SDRELHLSWEV

SEQ ID NO: 602 - protein, 10 aa, Severe acute respiratory syndrome coronavirus 2
SELVIGAVIL

SEQ ID NO: 603 - protein, 11 aa, Severe acute respiratory syndrome coronavirus 2
KEITVATSRTL

SEQ ID NO: 604 - protein, 9 aa, Severe acute respiratory syndrome coronavirus 2
VEKGVLPQL

SEQ ID NO: 605 - protein, 12 aa, Severe acute respiratory syndrome coronavirus 2
GEIPVAYRKVLL

SEQ ID NO: 606 - protein, 10 aa, Severe acute respiratory syndrome coronavirus 2
TEVPANSTVL

SEQ ID NO: 607 - protein, 10 aa, Severe acute respiratory syndrome coronavirus 2
REAVGTNLPL

SEQ ID NO: 608 - protein, 10 aa, Severe acute respiratory syndrome coronavirus 2
IEYPIIGDEL

SEQ ID NO: 609 - protein, 10 aa, Severe acute respiratory syndrome coronavirus 2
VELFENKTTL

SEQ ID NO: 610 - protein, 9 aa, Severe acute respiratory syndrome coronavirus 2
QEFKPRSQM

SEQ ID NO: 611 - protein, 10 aa, Severe acute respiratory syndrome coronavirus 2
MEIDFLELAM

SEQ ID NO: 612 - protein, 9 aa, Severe acute respiratory syndrome coronavirus 2
FELEDFIPM

SEQ ID NO: 613 - protein, 9 aa, Severe acute respiratory syndrome coronavirus 2
VEIIKSQDL

SEQ ID NO: 614 - protein, 11 aa, Severe acute respiratory syndrome coronavirus 2
YELQTPFEIKL

SEQ ID NO: 615 - protein, 13 aa, Severe acute respiratory syndrome coronavirus 2
ACHNSEVGPEHSL

SEQ ID NO: 616 - protein, 9 aa, Severe acute respiratory syndrome coronavirus 2
AEYHNESGL

SEQ ID NO: 617 - protein, 9 aa, Severe acute respiratory syndrome coronavirus 2
NESGLKTIL

SEQ ID NO: 618 - protein, 9 aa, Severe acute respiratory syndrome coronavirus 2
SEGLNDNLL

SEQ ID NO: 619 - protein, 10 aa, Severe acute respiratory syndrome coronavirus 2
GEQKSILSPL

SEQ ID NO: 620 - protein, 10 aa, Severe acute respiratory syndrome coronavirus 2
KEIKESVQTF

SEQ ID NO: 621 - protein, 11 aa, Severe acute respiratory syndrome coronavirus 2
KEIIFLEGETL

SEQ ID NO: 622 - protein, 9 aa, Severe acute respiratory syndrome coronavirus 2
GETLPTEVL

SEQ ID NO: 623 - protein, 11 aa, Severe acute respiratory syndrome coronavirus 2
FSSEIIGYKAI

SEQ ID NO: 624 - protein, 10 aa, Severe acute respiratory syndrome coronavirus 2
REVGFVVPGL

SEQ ID NO: 625 - protein, 13 aa, Severe acute respiratory syndrome coronavirus 2
GEYSHVVAFNTLL

SEQ ID NO: 626 - protein, 12 aa, Severe acute respiratory syndrome coronavirus 2
FLYENAFLPFAM

SEQ ID NO: 627 - protein, 10 aa, Severe acute respiratory syndrome coronavirus 2
TEAFEKMVSL

SEQ ID NO: 628 - protein, 10 aa, Severe acute respiratory syndrome coronavirus 2
KSEFDRDAAM

SEQ ID NO: 629 - protein, 11 aa, Severe acute respiratory syndrome coronavirus 2
SEISMDNSPNL

SEQ ID NO: 630 - protein, 10 aa, Severe acute respiratory syndrome coronavirus 2
QECVRGTTVL

SEQ ID NO: 631 - protein, 10 aa, Severe acute respiratory syndrome coronavirus 2
YEGNSPFHPL

SEQ ID NO: 632 - protein, 9 aa, Severe acute respiratory syndrome coronavirus 2
QELYSPIFL

SEQ ID NO: 633 - protein, 14 aa, Severe acute respiratory syndrome coronavirus 2
YEDFLEYHDVRVVL

SEQ ID NO: 634 - protein, 9 aa, Severe acute respiratory syndrome coronavirus 2
IEVQGYKSV

SEQ ID NO: 635 - protein, 11 aa, Severe acute respiratory syndrome coronavirus 2
FELDERIDKVL

SEQ ID NO: 636 - protein, 9 aa, Severe acute respiratory syndrome coronavirus 2
SELLTPLGI

SEQ ID NO: 637 - protein, 9 aa, Severe acute respiratory syndrome coronavirus 2
SEDNQTTTI

SEQ ID NO: 638 - protein, 9 aa, Severe acute respiratory syndrome coronavirus 2
VEVQPQLEM

SEQ ID NO: 639 - protein, 12 aa, Severe acute respiratory syndrome coronavirus 2
LEMELTPVVQTI

SEQ ID NO: 640 - protein, 10 aa, Severe acute respiratory syndrome coronavirus 2
YENFNQHEVL

SEQ ID NO: 641 - protein, 9 aa, Severe acute respiratory syndrome coronavirus 2
HEVLLAPLL

SEQ ID NO: 642 - protein, 9 aa, Severe acute respiratory syndrome coronavirus 2
NETLVTMPL

SEQ ID NO: 643 - protein, 9 aa, Severe acute respiratory syndrome coronavirus 2
GEVITFDNL

SEQ ID NO: 644 - protein, 11 aa, Severe acute respiratory syndrome coronavirus 2
SLREVRTIKVF

SEQ ID NO: 645 - protein, 9 aa, Severe acute respiratory syndrome coronavirus 2
HEGKTFYVL

SEQ ID NO: 646 - protein, 10 aa, Severe acute respiratory syndrome coronavirus 2
IELKFNPPAL

SEQ ID NO: 647 - protein, 9 aa, Severe acute respiratory syndrome coronavirus 2
GEAANFCAL

SEQ ID NO: 648 - protein, 10 aa, Severe acute respiratory syndrome coronavirus 2
YEQFKKGVQI

SEQ ID NO: 649 - protein, 12 aa, Severe acute respiratory syndrome coronavirus 2
SEYKGPITDVFY

SEQ ID NO: 650 - protein, 9 aa, Severe acute respiratory syndrome coronavirus 2
KENSYTTTI

SEQ ID NO: 651 - protein, 9 aa, Severe acute respiratory syndrome coronavirus 2
TEVVGDIIL

SEQ ID NO: 652 - protein, 9 aa, Severe acute respiratory syndrome coronavirus 2
NELSRVLGL

SEQ ID NO: 653 - protein, 10 aa, Severe acute respiratory syndrome coronavirus 2
AEWFLAYILF

SEQ ID NO: 654 - protein, 11 aa, Severe acute respiratory syndrome coronavirus 2
AEAELAKNVSL

SEQ ID NO: 655 - protein, 13 aa, Severe acute respiratory syndrome coronavirus 2
AELAKNVSLDNVL

SEQ ID NO: 656 - protein, 9 aa, Severe acute respiratory syndrome coronavirus 2
VENMTPRDL

SEQ ID NO: 657 - protein, 9 aa, Severe acute respiratory syndrome coronavirus 2
HEETIYNLL

SEQ ID NO: 658 - protein, 9 aa, Severe acute respiratory syndrome coronavirus 2
AESHVDTDL

SEQ ID NO: 659 - protein, 10 aa, Severe acute respiratory syndrome coronavirus 2
HEFCSQHTML

SEQ ID NO: 660 - protein, 11 aa, Severe acute respiratory syndrome coronavirus 2
YEAMYTPHTVL

SEQ ID NO: 661 - protein, 9 aa, Severe acute respiratory syndrome coronavirus 2
FEYVSQPFL

SEQ ID NO: 662 - protein, 9 aa, Severe acute respiratory syndrome coronavirus 2
GEVFNATRF

SEQ ID NO: 663 - protein, 10 aa, Severe acute respiratory syndrome coronavirus 2
VEAEVQIDRL

SEQ ID NO: 664 - protein, 9 aa, Severe acute respiratory syndrome coronavirus 2
AEVQIDRLI

SEQ ID NO: 665 - protein, 9 aa, Severe acute respiratory syndrome coronavirus 2
AEIRASANL

SEQ ID NO: 666 - protein, 9 aa, Severe acute respiratory syndrome coronavirus 2
KEIDRLNEV

SEQ ID NO: 667 - protein, 13 aa, Severe acute respiratory syndrome coronavirus 2
REVLSDRELHLSW

SEQ ID NO: 668 - protein, 10 aa, Severe acute respiratory syndrome coronavirus 2
GEYTFEKGDY

SEQ ID NO: 669 - protein, 9 aa, Severe acute respiratory syndrome coronavirus 2
DEISMATNY

SEQ ID NO: 670 - protein, 10 aa, Severe acute respiratory syndrome coronavirus 2
REFLTRNPAW

SEQ ID NO: 671 - protein, 10 aa, Severe acute respiratory syndrome coronavirus 2
EELKKLLEQW

SEQ ID NO: 672 - protein, 8 aa, Severe acute respiratory syndrome coronavirus 2
AELEGIQY

SEQ ID NO: 673 - protein, 8 aa, Severe acute respiratory syndrome coronavirus 2
AENVTGLF

SEQ ID NO: 674 - protein, 11 aa, Severe acute respiratory syndrome coronavirus 2
REEAIRHVRAW

SEQ ID NO: 675 - protein, 9 aa, Severe acute respiratory syndrome coronavirus 2
FELTSMKYF

SEQ ID NO: 676 - protein, 10 aa, Severe acute respiratory syndrome coronavirus 2
GEAVKTQFNY

SEQ ID NO: 677 - protein, 8 aa, Severe acute respiratory syndrome coronavirus 2
TEISFMLW

SEQ ID NO: 678 - protein, 12 aa, Severe acute respiratory syndrome coronavirus 2
KENDSKEGFFTY

SEQ ID NO: 679 - protein, 10 aa, Severe acute respiratory syndrome coronavirus 2
REHEHEIAWY

SEQ ID NO: 680 - protein, 9 aa, Severe acute respiratory syndrome coronavirus 2
FEIKLAKKF

SEQ ID NO: 681 - protein, 12 aa, Severe acute respiratory syndrome coronavirus 2
SEVGPEHSLAEY

SEQ ID NO: 682 - protein, 10 aa, Severe acute respiratory syndrome coronavirus 2
EEIAIILASF

SEQ ID NO: 683 - protein, 10 aa, Severe acute respiratory syndrome coronavirus 2
YEKLKPVLDW

SEQ ID NO: 684 - protein, 10 aa, Severe acute respiratory syndrome coronavirus 2
EEKFKEGVEF

SEQ ID NO: 685 - protein, 9 aa, Severe acute respiratory syndrome coronavirus 2
LEIKDTEKY

SEQ ID NO: 686 - protein, 13 aa, Severe acute respiratory syndrome coronavirus 2
SEAGVCVSTSGRW

SEQ ID NO: 687 - protein, 10 aa, Severe acute respiratory syndrome coronavirus 2
ASEFSSLPSY

SEQ ID NO: 688 - protein, 9 aa, Severe acute respiratory syndrome coronavirus 2
MDNSPNLAW

SEQ ID NO: 689 - protein, 9 aa, Severe acute respiratory syndrome coronavirus 2
TELEPPCRF

SEQ ID NO: 690 - protein, 13 aa, Severe acute respiratory syndrome coronavirus 2
QELIRQGTDYKHW

SEQ ID NO: 691 - protein, 13 aa, Severe acute respiratory syndrome coronavirus 2
SRIGMEVTPSGTW

SEQ ID NO: 692 - protein, 11 aa, Severe acute respiratory syndrome coronavirus 2
GMEVTPSGTWL

SEQ ID NO: 693 - protein, 10 aa, Severe acute respiratory syndrome coronavirus 2
GLEAPFLYLY

SEQ ID NO: 694 - protein, 14 aa, Severe acute respiratory syndrome coronavirus 2
SEHDYQIGGYTEKW

SEQ ID NO: 695 - protein, 9 aa, Severe acute respiratory syndrome coronavirus 2
EEHVQIHTI

SEQ ID NO: 696 - protein, 9 aa, Severe acute respiratory syndrome coronavirus 2
DEWSMATYY

SEQ ID NO: 697 - protein, 10 aa, Severe acute respiratory syndrome coronavirus 2
GEFKLASHMY

SEQ ID NO: 698 - protein, 14 aa, Severe acute respiratory syndrome coronavirus 2
GDCEEEEFEPSTQY

SEQ ID NO: 699 - protein, 12 aa, Severe acute respiratory syndrome coronavirus 2
EEEFEPSTQYEY

SEQ ID NO: 700 - protein, 9 aa, Severe acute respiratory syndrome coronavirus 2
IEVNSFSGY

SEQ ID NO: 701 - protein, 9 aa, Severe acute respiratory syndrome coronavirus 2
SEKQVEQKI

SEQ ID NO: 702 - protein, 11 aa, Severe acute respiratory syndrome coronavirus 2
AEIPKEEVKPF

SEQ ID NO: 703 - protein, 13 aa, Severe acute respiratory syndrome coronavirus 2
SNEKQEILGTVSW

SEQ ID NO: 704 - protein, 11 aa, Severe acute respiratory syndrome coronavirus 2
QEGVVDYGARF

SEQ ID NO: 705 - protein, 12 aa, Severe acute respiratory syndrome coronavirus 2
GELGDVRETMSY

SEQ ID NO: 706 - protein, 12 aa, Severe acute respiratory syndrome coronavirus 2
SEYTGNYQCGHY

SEQ ID NO: 707 - protein, 11 aa, Severe acute respiratory syndrome coronavirus 2
TEIDPKLDNYY

SEQ ID NO: 708 - protein, 11 aa, Severe acute respiratory syndrome coronavirus 2
AEVAVKMFDAY

SEQ ID NO: 709 - protein, 14 aa, Severe acute respiratory syndrome coronavirus 2
TVYSDVENPHLMGW

SEQ ID NO: 710 - protein, 11 aa, Severe acute respiratory syndrome coronavirus 2
SDVENPHLMGW

SEQ ID NO: 711 - protein, 10 aa, Severe acute respiratory syndrome coronavirus 2
TEKSNIIRGW

SEQ ID NO: 712 - protein, 10 aa, Severe acute respiratory syndrome coronavirus 2
SETKCTLKSF

SEQ ID NO: 713 - protein, 11 aa, Severe acute respiratory syndrome coronavirus 2
QEVFAQVKQIY

SEQ ID NO: 714 - protein, 9 aa, Severe acute respiratory syndrome coronavirus 2
ADAGFIKQY

SEQ ID NO: 715 - protein, 12 aa, Severe acute respiratory syndrome coronavirus 2
REGVFVSNGTHW

SEQ ID NO: 716 - protein, 12 aa, Severe acute respiratory syndrome coronavirus 2
QELGKYEQYIKW

SEQ ID NO: 717 - protein, 9 aa, Severe acute respiratory syndrome coronavirus 2
YEQYIKWPW

SEQ ID NO: 718 - protein, 12 aa, Severe acute respiratory syndrome coronavirus 2
AEIVDTVSALVY

SEQ ID NO: 719 - protein, 11 aa, Severe acute respiratory syndrome coronavirus 2
RELNGGAYTRY

SEQ ID NO: 720 - protein, 11 aa, Severe acute respiratory syndrome coronavirus 2
SYEDQDALFAY

SEQ ID NO: 721 - protein, 8 aa, Severe acute respiratory syndrome coronavirus 2
AEHVNNSY

SEQ ID NO: 722 - protein, 9 aa, Severe acute respiratory syndrome coronavirus 2
TTDLEGNFY

SEQ ID NO: 723 - protein, 9 aa, Severe acute respiratory syndrome coronavirus 2
TVDSQGSEY

SEQ ID NO: 724 - protein, 9 aa, Severe acute respiratory syndrome coronavirus 2
ASDSGFAAY

SEQ ID NO: 725 - protein, 9 aa, Severe acute respiratory syndrome coronavirus 2
VADSGFAAY

SEQ ID NO: 726 - protein, 9 aa, Severe acute respiratory syndrome coronavirus 2
LADEFIERY

SEQ ID NO: 727 - protein, 9 aa, Severe acute respiratory syndrome coronavirus 2
YVDNGPDGY

SEQ ID NO: 728 - protein, 9 aa, Severe acute respiratory syndrome coronavirus 2
TSDLATNAY

SEQ ID NO: 729 - protein, 9 aa, Severe acute respiratory syndrome coronavirus 2
HTDFSSEIY

SEQ ID NO: 730 - protein, 9 aa, Severe acute respiratory syndrome coronavirus 2
FSNSGSDLY

SEQ ID NO: 731 - protein, 9 aa, Severe acute respiratory syndrome coronavirus 2
NTDGTTFTY

SEQ ID NO: 732 - protein, 9 aa, Severe acute respiratory syndrome coronavirus 2
TTDDNALAY

SEQ ID NO: 733 - protein, 9 aa, Severe acute respiratory syndrome coronavirus 2
QTDNALAYY

SEQ ID NO: 734 - protein, 9 aa, Severe acute respiratory syndrome coronavirus 2
CTDDNALYY

SEQ ID NO: 735 - protein, 9 aa, Severe acute respiratory syndrome coronavirus 2
VTDTPKYLY

SEQ ID NO: 736 - protein, 9 aa, Severe acute respiratory syndrome coronavirus 2
STDTGVEHY

SEQ ID NO: 737 - protein, 9 aa, Severe acute respiratory syndrome coronavirus 2
VVDDPIHFY

SEQ ID NO: 738 - protein, 9 aa, Severe acute respiratory syndrome coronavirus 2
ATDNYITTY

SEQ ID NO: 739 - protein, 9 aa, Severe acute respiratory syndrome coronavirus 2
TTDPSFLRY

SEQ ID NO: 740 - protein, 9 aa, Severe acute respiratory syndrome coronavirus 2
ITDYKENSY

SEQ ID NO: 741 - protein, 9 aa, Severe acute respiratory syndrome coronavirus 2
FTEQPIDLY

SEQ ID NO: 742 - protein, 9 aa, Severe acute respiratory syndrome coronavirus 2
FADDLNQLY

SEQ ID NO: 743 - protein, 9 aa, Severe acute respiratory syndrome coronavirus 2
ITEEVGHAY

SEQ ID NO: 744 - protein, 9 aa, Severe acute respiratory syndrome coronavirus 2
STDRAFDIY

SEQ ID NO: 745 - protein, 9 aa, Severe acute respiratory syndrome coronavirus 2
ASDYDYYRY

SEQ ID NO: 746 - protein, 9 aa, Severe acute respiratory syndrome coronavirus 2
YSDVENPHY

SEQ ID NO: 747 - protein, 9 aa, Severe acute respiratory syndrome coronavirus 2
STDGIADKY

SEQ ID NO: 748 - protein, 9 aa, Severe acute respiratory syndrome coronavirus 2
NTDFVNEFY

SEQ ID NO: 749 - protein, 9 aa, Severe acute respiratory syndrome coronavirus 2
LSDDANSTY

SEQ ID NO: 750 - protein, 9 aa, Severe acute respiratory syndrome coronavirus 2
YTNDNTSRY

SEQ ID NO: 751 - protein, 9 aa, Severe acute respiratory syndrome coronavirus 2
LADAGFIQY

SEQ ID NO: 752 - protein, 9 aa, Severe acute respiratory syndrome coronavirus 2
VTDEMIAQY

SEQ ID NO: 753 - protein, 9 aa, Severe acute respiratory syndrome coronavirus 2
FLVAGNVQL

SEQ ID NO: 754 - protein, 9 aa, Severe acute respiratory syndrome coronavirus 2
ALLEDEFTV

SEQ ID NO: 755 - protein, 9 aa, Severe acute respiratory syndrome coronavirus 2
LLLDDFVEI

SEQ ID NO: 756 - protein, 9 aa, Severe acute respiratory syndrome coronavirus 2
SLFDFPLKL

SEQ ID NO: 757 - protein, 9 aa, Severe acute respiratory syndrome coronavirus 2
GLNDNLLEL

SEQ ID NO: 758 - protein, 9 aa, Severe acute respiratory syndrome coronavirus 2
YLDGADVTI

SEQ ID NO: 759 - protein, 9 aa, Severe acute respiratory syndrome coronavirus 2
YMADLVYAL

SEQ ID NO: 760 - protein, 9 aa, Severe acute respiratory syndrome coronavirus 2
KIFDGVPFV

SEQ ID NO: 761 - protein, 9 aa, Severe acute respiratory syndrome coronavirus 2
SMQGAVINK

SEQ ID NO: 762 - protein, 9 aa, Severe acute respiratory syndrome coronavirus 2
KLKKSLVAK

SEQ ID NO: 763 - protein, 9 aa, Severe acute respiratory syndrome coronavirus 2
KLMPVVETK

SEQ ID NO: 764 - protein, 9 aa, Severe acute respiratory syndrome coronavirus 2
AVYMGTLSY

SEQ ID NO: 765 - protein, 9 aa, Severe acute respiratory syndrome coronavirus 2
LLKDPAVAK

SEQ ID NO: 766 - protein, 9 aa, Severe acute respiratory syndrome coronavirus 2
RIAPGQTGK

SEQ ID NO: 767 - protein, 9 aa, Severe acute respiratory syndrome coronavirus 2
KIYKTPPIK

SEQ ID NO: 768 - protein, 9 aa, Severe acute respiratory syndrome coronavirus 2
STSAFVTVK

SEQ ID NO: 769 - protein, 9 aa, Severe acute respiratory syndrome coronavirus 2
AVANGVVLK

SEQ ID NO: 770 - protein, 9 aa, Severe acute respiratory syndrome coronavirus 2
VVNAANVYK

SEQ ID NO: 771 - protein, 9 aa, Severe acute respiratory syndrome coronavirus 2
STLGIEFLK

SEQ ID NO: 772 - protein, 9 aa, Severe acute respiratory syndrome coronavirus 2
TILPVSMTK

SEQ ID NO: 773 - protein, 9 aa, Severe acute respiratory syndrome coronavirus 2
VFFDGRVLF

SEQ ID NO: 774 - protein, 9 aa, Severe acute respiratory syndrome coronavirus 2
GYINVFAFF

SEQ ID NO: 775 - protein, 9 aa, Severe acute respiratory syndrome coronavirus 2
NYSGVVTTF

SEQ ID NO: 776 - protein, 9 aa, Severe acute respiratory syndrome coronavirus 2
YYAGEAANF

SEQ ID NO: 777 - protein, 9 aa, Severe acute respiratory syndrome coronavirus 2
MYMGSYEQF

SEQ ID NO: 778 - protein, 9 aa, Severe acute respiratory syndrome coronavirus 2
RFPNITNLF

SEQ ID NO: 779 - protein, 9 aa, Severe acute respiratory syndrome coronavirus 2
AYIAIPTNF

SEQ ID NO: 780 - protein, 9 aa, Severe acute respiratory syndrome coronavirus 2
SPSGVYQAM

SEQ ID NO: 781 - protein, 9 aa, Severe acute respiratory syndrome coronavirus 2
RPNTIKGSF

SEQ ID NO: 782 - protein, 9 aa, Severe acute respiratory syndrome coronavirus 2
KPRPPLNVF

SEQ ID NO: 783 - protein, 9 aa, Severe acute respiratory syndrome coronavirus 2
IPARARVEF

SEQ ID NO: 784 - protein, 9 aa, Severe acute respiratory syndrome coronavirus 2
RPQIGVVEF

SEQ ID NO: 785 - protein, 9 aa, Severe acute respiratory syndrome coronavirus 2
SPYNSQNAL

SEQ ID NO: 786 - protein, 9 aa, Severe acute respiratory syndrome coronavirus 2
APHGHVMVL

SEQ ID NO: 787 - protein, 9 aa, Severe acute respiratory syndrome coronavirus 2
VPANSTVSF

SEQ ID NO: 788 - protein, 9 aa, Severe acute respiratory syndrome coronavirus 2
HPNPKGFDL

SEQ ID NO: 789 - protein, 9 aa, Severe acute respiratory syndrome coronavirus 2
GPKQAGVTL

SEQ ID NO: 790 - protein, 9 aa, Severe acute respiratory syndrome coronavirus 2
VPGLPGTIL

SEQ ID NO: 791 - protein, 9 aa, Severe acute respiratory syndrome coronavirus 2
VPYYDTNVL

SEQ ID NO: 792 - protein, 9 aa, Severe acute respiratory syndrome coronavirus 2
TPIQPIGAL

SEQ ID NO: 793 - protein, 9 aa, Severe acute respiratory syndrome coronavirus 2
KPRGQGVPI

SEQ ID NO: 794 - protein, 9 aa, Severe acute respiratory syndrome coronavirus 2
SPFHPLAAL

SEQ ID NO: 795 - protein, 9 aa, Severe acute respiratory syndrome coronavirus 2
YPQNGLTSI

SEQ ID NO: 796 - protein, 9 aa, Severe acute respiratory syndrome coronavirus 2
KPRELKVTF

SEQ ID NO: 797 - protein, 9 aa, Severe acute respiratory syndrome coronavirus 2
TPFKKGAKL

SEQ ID NO: 798 - protein, 9 aa, Severe acute respiratory syndrome coronavirus 2
KPNELSRVL

SEQ ID NO: 799 - protein, 9 aa, Severe acute respiratory syndrome coronavirus 2
MPILTTRAL

SEQ ID NO: 800 - protein, 9 aa, Severe acute respiratory syndrome coronavirus 2
FPLANGQVF

SEQ ID NO: 801 - protein, 9 aa, Severe acute respiratory syndrome coronavirus 2
YPLNISDEF

SEQ ID NO: 802 - protein, 9 aa, Severe acute respiratory syndrome coronavirus 2
IPIPKDMTY

SEQ ID NO: 803 - protein, 9 aa, Severe acute respiratory syndrome coronavirus 2
YPANSIVRF

SEQ ID NO: 804 - protein, 9 aa, Severe acute respiratory syndrome coronavirus 2
IPMDSTVKY

SEQ ID NO: 805 - protein, 9 aa, Severe acute respiratory syndrome coronavirus 2
FPDGVKHVY

SEQ ID NO: 806 - protein, 8 aa, Severe acute respiratory syndrome coronavirus 2
SPDAVTAY

SEQ ID NO: 807 - protein, 9 aa, Severe acute respiratory syndrome coronavirus 2
LPNDDTLEY

SEQ ID NO: 808 - protein, 9 aa, Severe acute respiratory syndrome coronavirus 2
YPLTPNQEY

SEQ ID NO: 809 - protein, 9 aa, Severe acute respiratory syndrome coronavirus 2
LPIGINITF

SEQ ID NO: 810 - protein, 9 aa, Severe acute respiratory syndrome coronavirus 2
IPIGAGISY

SEQ ID NO: 811 - protein, 9 aa, Severe acute respiratory syndrome coronavirus 2
FPAPHGVVF

SEQ ID NO: 812 - protein, 9 aa, Severe acute respiratory syndrome coronavirus 2
SEVIGAVIL

SEQ ID NO: 813 - protein, 9 aa, Severe acute respiratory syndrome coronavirus 2
KEITVATTL

SEQ ID NO: 814 - protein, 9 aa, Severe acute respiratory syndrome coronavirus 2
TEVPANTVL

SEQ ID NO: 815 - protein, 9 aa, Severe acute respiratory syndrome coronavirus 2
REAVGTNLL

SEQ ID NO: 816 - protein, 9 aa, Severe acute respiratory syndrome coronavirus 2
IEYPIGDEL

SEQ ID NO: 817 - protein, 9 aa, Severe acute respiratory syndrome coronavirus 2
MEIDFELAM

SEQ ID NO: 818 - protein, 9 aa, Severe acute respiratory syndrome coronavirus 2
YELQTPIKL

SEQ ID NO: 819 - protein, 9 aa, Severe acute respiratory syndrome coronavirus 2
AEVGPEHSL

SEQ ID NO: 820 - protein, 9 aa, Severe acute respiratory syndrome coronavirus 2
GEQKSILSL

SEQ ID NO: 821 - protein, 9 aa, Severe acute respiratory syndrome coronavirus 2
KEFLEGETL

SEQ ID NO: 822 - protein, 9 aa, Severe acute respiratory syndrome coronavirus 2
REVGFVVGL

SEQ ID NO: 823 - protein, 9 aa, Severe acute respiratory syndrome coronavirus 2
QEVRGTTVL

SEQ ID NO: 824 - protein, 9 aa, Severe acute respiratory syndrome coronavirus 2
YENSPFHPL

SEQ ID NO: 825 - protein, 9 aa, Severe acute respiratory syndrome coronavirus 2
LEMELTPVV

SEQ ID NO: 826 - protein, 9 aa, Severe acute respiratory syndrome coronavirus 2
YEFNQHEVL

SEQ ID NO: 827 - protein, 9 aa, Severe acute respiratory syndrome coronavirus 2
YEFKKGVQI

SEQ ID NO: 828 - protein, 9 aa, Severe acute respiratory syndrome coronavirus 2
HEFSQHTML

SEQ ID NO: 829 - protein, 9 aa, Severe acute respiratory syndrome coronavirus 2
YEYTPHTVL

SEQ ID NO: 830 - protein, 9 aa, Severe acute respiratory syndrome coronavirus 2
VEAEVQIRL

SEQ ID NO: 831 - protein, 9 aa, Severe acute respiratory syndrome coronavirus 2
SEAGVSGRW

SEQ ID NO: 832 - protein, 9 aa, Severe acute respiratory syndrome coronavirus 2
QELTDYKHW

SEQ ID NO: 833 - protein, 9 aa, Severe acute respiratory syndrome coronavirus 2
SEVTPSGTW

SEQ ID NO: 834 - protein, 9 aa, Severe acute respiratory syndrome coronavirus 2
TENPHLMGW

SEQ ID NO: 835 - protein, 9 aa, Severe acute respiratory syndrome coronavirus 2
RELNGGARY

SEQ ID NO: 836 - protein, 10 aa, Severe acute respiratory syndrome coronavirus 2
TTDPSFLGRY

SEQ ID NO: 837 - protein, 275 aa, Homo sapiens
GSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDRRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWK

SEQ ID NO: 838 - protein, 275 aa, synthetic construct
GSHSLKYFHTSVSRPGRGEPRFISVGYVDDTQFVRFDNDAASPRMVPRAPWMEQEGSEYWDRETRSARDTAQIFRVNLRTLRGCYNQSEAGSHTLQWMHGCELGPDRRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPCGDGTFQKWAAVVVPSGEEQRYTCHVQHEGLPEPVTLRWK

SEQ ID NO: 839 - protein, 275 aa, Homo sapiens
GSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWK

SEQ ID NO: 840 - protein, 275 aa, synthetic construct
GSHSLKYFHTSVSRPGRGEPRFISVGYVDDTQFVRFDNDAASPRMVPRAPWMEQEGSEYWDRETRSARDTAQIFRVNLRTLRGCYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPCGDGTFQKWAAVVVPSGEEQRYTCHVQHEGLPEPVTLRWK

SEQ ID NO: 841 - protein, 275 aa, Homo sapiens
GSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWK

SEQ ID NO: 842 - protein, 275 aa, synthetic construct
GSHSMRYFSAAVSRPGRGEPRFIAMGYVDDTQFVRFDSDSACPRMEPRAPWVEQEGPEYWEEETRNTKAHAQTDRMNLQTLRGCYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPCGDGTFQKWAAVVVPSGEEQRYTCHVQHEGLPEPLMLRWK

SEQ ID NO: 843 - protein, 275 aa, Homo sapiens
GSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLIRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWK

SEQ ID NO: 844 - protein, 275 aa, synthetic construct
GSHSMRYFSAAVSRPGRGEPRFIAMGYVDDTQFVRFDSDSACPRMEPRAPWVEQEGPEYWEEETRNTKAHAQTDRMNLQTLRGCYNQSEASSHTLQWMIGCDLGSDGRLIRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPCGDGTFQKWAAVVVPSGEEQRYTCHVQHEGLPEPLMLRWK

SEQ ID NO: 845 - protein, 10 aa, Severe acute respiratory syndrome coronavirus 2
STQDLFLPFF

SEQ ID NO: 846 - protein, 11 aa, Severe acute respiratory syndrome coronavirus 2
FCNDPFLGVYY

SEQ ID NO: 847 - protein, 10 aa, Severe acute respiratory syndrome coronavirus 2
SSANNCTFEY

SEQ ID NO: 848 - protein, 11 aa, Severe acute respiratory syndrome coronavirus 2
STECSNLLLQY

SEQ ID NO: 849 - protein, 10 aa, Severe acute respiratory syndrome coronavirus 2
YTNSFTRGVY

SEQ ID NO: 850 - protein, 9 aa, Severe acute respiratory syndrome coronavirus 2
CVADYSVLY

SEQ ID NO: 851 - protein, 9 aa, Severe acute respiratory syndrome coronavirus 2
LTDEMIAQY

SEQ ID NO: 852 - protein, 10 aa, Severe acute respiratory syndrome coronavirus 2
KLPDDFTGCV

SEQ ID NO: 853 - protein, 11 aa, Severe acute respiratory syndrome coronavirus 2
SFELLHAPATV

SEQ ID NO: 854 - protein, 9 aa, Severe acute respiratory syndrome coronavirus 2
RLNEVAKNL

SEQ ID NO: 855 - protein, 9 aa, Severe acute respiratory syndrome coronavirus 2
NLNESLIDL

SEQ ID NO: 856 - protein, 9 aa, Severe acute respiratory syndrome coronavirus 2
VVFLHVTYV

SEQ ID NO: 857 - protein, 9 aa, Severe acute respiratory syndrome coronavirus 2
KCYGVSPTK

SEQ ID NO: 858 - protein, 12 aa, Severe acute respiratory syndrome coronavirus 2
VYDPLQPELDSF

SEQ ID NO: 859 - protein, 11 aa, Severe acute respiratory syndrome coronavirus 2
YEQYIKWPWYI

SEQ ID NO: 860 - protein, 10 aa, Severe acute respiratory syndrome coronavirus 2
KWPWYIWLGF

SEQ ID NO: 861 - protein, 9 aa, Severe acute respiratory syndrome coronavirus 2
GTITSGWTF

SEQ ID NO: 862 - protein, 10 aa, Severe acute respiratory syndrome coronavirus 2
EILDITPCSF

SEQ ID NO: 863 - protein, 10 aa, Severe acute respiratory syndrome coronavirus 2
SVASQSIIAY

SEQ ID NO: 864 - protein, 10 aa, Severe acute respiratory syndrome coronavirus 2
APHGVVFLHV

SEQ ID NO: 865 - protein, 9 aa, Severe acute respiratory syndrome coronavirus 2
MIAQYTSAL

SEQ ID NO: 866 - protein, 10 aa, Severe acute respiratory syndrome coronavirus 2
TECSNLLLQY

SEQ ID NO: 867 - protein, 1062 aa, synthetic construct
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 868 - protein, 1062 aa, synthetic construct
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSGSHSMRYFYTSVSRPGRGEPRFIAVGYVDDTQFVRFDSDAASQRMEPRAPWIEQEGPEYWDQETRNVKAQSQTDRVDLGTLRGCYNQSEDGSHTIQIMYGCDVGPDGRFLRGYRQDAYDGKDYIALNEDLRSWTAADMCAQITKRKWEAAHAAEQQRAYLEGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 869 - protein, 1062 aa, synthetic construct
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSGSHSMRYFSTSVSRPGRGEPRFIAVGYVDDTQFVRFDSDAASQRMEPRAPWIEQEGPEYWDEETGKVKAHSQTDRENLRIALRCYNQSEAGSHTLQMMFGCDVGSDGRFLRGYHQYAYDGKDYIALKEDLRSWTAADMCAQITKRKWEAAHVAEQQRAYLEGTCVDGLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 870 - protein, 1062 aa, synthetic construct
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSGSHSLKYFHTSVSRPGRGEPRFISVGYVDDTQFVRFDNDAASPRMVPRAPWMEQEGSEYWDRETRSARDTAQIFRVNLRTLRGCYNQSEAGSHTLQWMHGCELGPDRRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPCGDGTFQKWAAVVVPSGEEQRYTCHVQHEGLPEPVTLRWKAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 871 - protein, 1062 aa, synthetic construct
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSGSHSLKYFHTSVSRPGRGEPRFISVGYVDDTQFVRFDNDAASPRMVPRAPWMEQEGSEYWDRETRSARDTAQIFRVNLRTLRGCYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPCGDGTFQKWAAVVVPSGEEQRYTCHVQHEGLPEPVTLRWKAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 872 - protein, 250 aa, synthetic construct
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSS

SEQ ID NO: 873 - protein, 5 aa, synthetic construct
GGGGS

SEQ ID NO: 874 - protein, 5 aa, synthetic construct
AAAGG

SEQ ID NO: 875 - protein, 15 aa, synthetic construct
GGGGSGGGGSGGGGS

SEQ ID NO: 876 - protein, 20 aa, synthetic construct
GGGGSGGGGSGGGGSGGGGS

SEQ ID NO: 877 - protein, 226 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 878 - protein, 133 aa, synthetic construct
APTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 879 - protein, 274 aa, synthetic construct
GSHSMRYFYTSVSRPGRGEPRFIAVGYVDDTQFVRFDSDAASQRMEPRAPWIEQEGPEYWDQETRNVKAQSQTDRVDLGTLRGCYNQSEDGSHTIQIMYGCDVGPDGRFLRGYRQDAYDGKDYIALNEDLRSWTAADMCAQITKRKWEAAHAAEQQRAYLEGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRW

SEQ ID NO: 880 - protein, 275 aa, synthetic construct
GSHSMRYFSTSVSRPGRGEPRFIAVGYVDDTQFVRFDSDAASQRMEPRAPWIEQEGPEYWDEETGKVKAHSQTDRENLRIALRCYNQSEAGSHTLQMMFGCDVGSDGRFLRGYHQYAYDGKDYIALKEDLRSWTAADMCAQITKRKWEAAHVAEQQRAYLEGTCVDGLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWE

SEQ ID NO: 881 - protein, 139 aa, synthetic construct
  VARIANT 1..25: note = Wherein X is any amino acid and up to 21 copies of X can be deleted
XXXXXXXXXXXXXXXXXXXXXXXXXGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 882 - protein, 15 aa, synthetic construct
GCGGSGGGGSGGGGS

SEQ ID NO: 883 - protein, 123 aa, synthetic construct
YLQPRTFLLGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 884 - protein, 123 aa, synthetic construct
GTHWFVTQRGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 885 - protein, 123 aa, synthetic construct
QYIKWPWYIGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 886 - protein, 123 aa, synthetic construct
YLQPRTLFLGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 887 - protein, 9 aa, synthetic construct
YLQPRTLFL

SEQ ID NO: 888 - protein, 248 aa, synthetic construct
QMQLVESGGGVVQPGRSLRLSCTASGFTFSNNGMHWVRQAPGKGLEWVAVIWFDGMNKFYVDSVKGRFTISRDNSKNTLYLEMNSLRAEDTAIYYCAREGDGSGIYYYYGMDVWGQGTTVTVSSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPIFTFGPGTKVDIK

SEQ ID NO: 889 - protein, 248 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPIFTFGPGTKVDIKGGGGSGGGGSGGGGSQMQLVESGGGVVQPGRSLRLSCTASGFTFSNNGMHWVRQAPGKGLEWVAVIWFDGMNKFYVDSVKGRFTISRDNSKNTLYLEMNSLRAEDTAIYYCAREGDGSGIYYYYGMDVWGQGTTVTVSS

SEQ ID NO: 890 - protein, 240 aa, synthetic construct
QVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGSGTPVTVSSGGGGSGGGGSGGGGSDIELTQSPAIMSASPGEKVTMTCSASSSVSYMHWYQQKSGTSPKRWIYDTSKLASGVPGRFSGSGSGNSYSLTISSVEAEDDATYYCQQWSKHPLTFGSGTKVEIK

SEQ ID NO: 891 - protein, 240 aa, synthetic construct
DIELTQSPAIMSASPGEKVTMTCSASSSVSYMHWYQQKSGTSPKRWIYDTSKLASGVPGRFSGSGSGNSYSLTISSVEAEDDATYYCQQWSKHPLTFGSGTKVEIKGGGGSGGGGSGGGGSQVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGSGTPVTVSS

SEQ ID NO: 892 - protein, 247 aa, synthetic construct
QVELVQSGAEVKKPGESLKISCKGSGYSFTSYWIGWVRQAPGKGLEWMGIIDPGDSRTRYSPSFQGQVTISADKSISTAYLQWSSLKASDTAMYYCARGQLYGGTYMDGWGQGTLVTVSSGGGGSGGGGSGGGGSDIALTQPASVSGSPGQSITISCTGTSSDIGGYNSVSWYQQHPGKAPKLMIYGVNNRPSGVSNRFSGSKSGNTASLTISGLQAEDEADYYCSSYDIESATPVFGGGTKLTVLG

SEQ ID NO: 893 - protein, 247 aa, synthetic construct
DIALTQPASVSGSPGQSITISCTGTSSDIGGYNSVSWYQQHPGKAPKLMIYGVNNRPSGVSNRFSGSKSGNTASLTISGLQAEDEADYYCSSYDIESATPVFGGGTKLTVLGGGGGSGGGGSGGGGSQVELVQSGAEVKKPGESLKISCKGSGYSFTSYWIGWVRQAPGKGLEWMGIIDPGDSRTRYSPSFQGQVTISADKSISTAYLQWSSLKASDTAMYYCARGQLYGGTYMDGWGQGTLVTVSS

SEQ ID NO: 894 - protein, 240 aa, synthetic construct
QVQLVQSGAEVKKPGASVKVSCKASGYSFTGYTMNWVRQAPGQGLEWMGLITPYNGASSYNQKFRGKATMTVDTSTSTVYMELSSLRSEDTAVYYCARGGYDGRGFDYWGQGTLVTVSSGGGGSGGGGSGGGGSDIQMTQSPSSLSASVGDRVTITCSASSSVSYMHWYQQKSGKAPKLLIYDTSKLASGVPSRFSGSGSGTDFTLTISSLQPEDFATYYCQQWSKHPLTFGQGTKLEIK

SEQ ID NO: 895 - protein, 240 aa, synthetic construct
DIQMTQSPSSLSASVGDRVTITCSASSSVSYMHWYQQKSGKAPKLLIYDTSKLASGVPSRFSGSGSGTDFTLTISSLQPEDFATYYCQQWSKHPLTFGQGTKLEIKGGGGSGGGGSGGGGSQVQLVQSGAEVKKPGASVKVSCKASGYSFTGYTMNWVRQAPGQGLEWMGLITPYNGASSYNQKFRGKATMTVDTSTSTVYMELSSLRSEDTAVYYCARGGYDGRGFDYWGQGTLVTVSS

SEQ ID NO: 896 - protein, 243 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSGGGGSGGGGSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIK

SEQ ID NO: 897 - protein, 243 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKGGGGSGGGGSGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 898 - protein, 243 aa, synthetic construct
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFDVWGQGTLVTVSSGGGGSGGGGSGGGGSDIQMTQSPSSLSASVGDRVTITCKASQDVSTAVAWYQQKPGKAPKLLIYSASYRYTGVPSRFSGSGSGTDFTLTISSLQPEDFAVYYCQQHYITPLTFGQGTKLEIK

SEQ ID NO: 899 - protein, 243 aa, synthetic construct
DIQMTQSPSSLSASVGDRVTITCKASQDVSTAVAWYQQKPGKAPKLLIYSASYRYTGVPSRFSGSGSGTDFTLTISSLQPEDFAVYYCQQHYITPLTFGQGTKLEIKGGGGSGGGGSGGGGSQVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFDVWGQGTLVTVSS

SEQ ID NO: 900 - protein, 881 aa, synthetic construct
  VARIANT 1..25: note = Wherein X is any amino acid and up to 21 copies of X can be deleted
XXXXXXXXXXXXXXXXXXXXXXXXXGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWEGSEVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 901 - protein, 121 aa, synthetic construct
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSS

SEQ ID NO: 902 - protein, 525 aa, synthetic construct
APTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSDIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 903 - protein, 113 aa, synthetic construct
DIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKR

SEQ ID NO: 904 - protein, 1182 aa, synthetic construct
  VARIANT 1..25: note = Wherein X is any amino acid and up to 21 copies of X can be deleted
XXXXXXXXXXXXXXXXXXXXXXXXXGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWEGSEVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 905 - protein, 219 aa, synthetic construct
DIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 906 - skipped

SEQ ID NO: 907 - protein, 520 aa, synthetic construct
DIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 908 - protein, 210 aa, synthetic construct
QWYQQKPGQPPKLLIFAASNVESGVPARFSGSGSGTNFSLNIHPVDEDDVAMYFCQQSRKVPYTFGGGTKLEIKRGGGGSGGGGSGGGGSQVKLQQSGPGLVTPSQSLSITCTVSGFSLSDYGVHWVRQSPGQGLEWLGVIWAGGGTNYNSALMSRKSISKDNSKSQVFLKMNSLQADDTAVYYCARDKGYSYYYSMDYWGQGTTVTVSS

SEQ ID NO: 909 - protein, 210 aa, synthetic construct
QVKLQQSGPGLVTPSQSLSITCTVSGFSLSDYGVHWVRQSPGQGLEWLGVIWAGGGTNYNSALMSRKSISKDNSKSQVFLKMNSLQADDTAVYYCARDKGYSYYYSMDYWGQGTTVTVSSGGGGSGGGGSGGGGSQWYQQKPGQPPKLLIFAASNVESGVPARFSGSGSGTNFSLNIHPVDEDDVAMYFCQQSRKVPYTFGGGTKLEIKR

SEQ ID NO: 910 - protein, 761 aa, synthetic construct
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 911 - protein, 807 aa, synthetic construct
GSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 912 - protein, 123 aa, synthetic construct
NLVPMVATVGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 913 - protein, 9 aa, synthetic construct
NLVPMVATV

SEQ ID NO: 914 - protein, 9 aa, Severe acute respiratory syndrome coronavirus 2
VMPLSAPTL

SEQ ID NO: 915 - protein, 5 aa, synthetic construct
GTLRG

SEQ ID NO: 916 - protein, 5 aa, synthetic construct
YNQSE

SEQ ID NO: 917 - protein, 5 aa, synthetic construct
TAADM

SEQ ID NO: 918 - protein, 5 aa, synthetic construct
AQTTK

SEQ ID NO: 919 - protein, 5 aa, synthetic construct
VETRP

SEQ ID NO: 920 - protein, 5 aa, synthetic construct
GDGTF

SEQ ID NO: 921 - protein, 5 aa, synthetic construct
RNLRG

SEQ ID NO: 922 - protein, 5 aa, synthetic construct
TAADT

SEQ ID NO: 923 - protein, 5 aa, synthetic construct
AQITQ

SEQ ID NO: 924 - protein, 5 aa, synthetic construct
GDRTF

SEQ ID NO: 925 - protein, 133 aa, synthetic construct
APTSSSTKKTQLQLETLLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 926 - protein, 167 aa, synthetic construct
DPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPA

SEQ ID NO: 927 - protein, 208 aa, synthetic construct [sequence omitted]

SEQ ID NO: 928 - protein, 224 aa, synthetic construct
APLKIQAYFNETADLPCQFANSQNQSLSELVVFWQDQENLVLNEVYLGKEKFDSVHSKYMNRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 929 - protein, 75 aa, synthetic construct
QWYQQKPGQPPKLLIFAASNVESGVPARFSGSGSGTNFSLNIHPVDEDDVAMYFCQQSRKVPYTFGGGTKLEIKR

SEQ ID NO: 930 - protein, 120 aa, synthetic construct
QVKLQQSGPGLVTPSQSLSITCTVSGFSLSDYGVHWVRQSPGQGLEWLGVIWAGGGTNYNSALMSRKSISKDNSKSQVFLKMNSLQADDTAVYYCARDKGYSYYYSMDYWGQGTTVTVSS

SEQ ID NO: 931 - protein, 5 aa, synthetic construct
DYGVH

SEQ ID NO: 932 - protein, 16 aa, synthetic construct
VIWAGGGTNYNSALMS

SEQ ID NO: 933 - protein, 12 aa, synthetic construct
DKGYSYYYSMDY

SEQ ID NO: 934 - protein, 50 aa, synthetic construct
  VARIANT 6..50: note = Wherein up to 9 copies of GGGGS can be omitted
GGGGSGGGGSGGGGSGGGGSGGGGSGGGGSGGGGSGGGGSGGGGSGGGGS

SEQ ID NO: 935 - protein, 5 aa, synthetic construct
  VARIANT 4: note = Wherein X is any amino acid other than P
VPGXG

SEQ ID NO: 936 - protein, 223 aa, synthetic construct
PPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPG

SEQ ID NO: 937 - protein, 20 aa, synthetic construct
  VARIANT 3..20: note = Wherein up to 9 copies of GS can be omitted
GSGSGSGSGSGSGSGSGSGS

SEQ ID NO: 938 - protein, 50 aa, synthetic construct
  VARIANT 6..50: note = Wherein up to 9 copies of GSGGS can be omitted
GSGGSGSGGSGSGGSGSGGSGSGGSGSGGSGSGGSGSGGSGSGGSGSGGS

SEQ ID NO: 939 - protein, 40 aa, synthetic construct
  VARIANT 5..40: note = Wherein up to 9 copies of GGGS can be omitted
GGGSGGGSGGGSGGGSGGGSGGGSGGGSGGGSGGGSGGGS

SEQ ID NO: 940 - protein, 4 aa, synthetic construct
GGSG

SEQ ID NO: 941 - protein, 5 aa, synthetic construct
GGSGG

SEQ ID NO: 942 - protein, 5 aa, synthetic construct
GSGSG

SEQ ID NO: 943 - protein, 5 aa, synthetic construct
GSGGG

SEQ ID NO: 944 - protein, 5 aa, synthetic construct
GGGSG

SEQ ID NO: 945 - protein, 5 aa, synthetic construct
GSSSG

SEQ ID NO: 946 - protein, 10 aa, synthetic construct
  VARIANT 2..10: note = Wherein up to 9 copies of G can be omitted
GGGGGGGGGG

SEQ ID NO: 947 - protein, 10 aa, synthetic construct
GGGGSGGGGS

SEQ ID NO: 948 - protein, 35 aa, synthetic construct
GGGGSGGGGSGGGGSGGGGSGGGGSGGGGSGGGGS

SEQ ID NO: 949 - protein, 100 aa, synthetic construct
  VARIANT 6..100: note = Wherein up to 19 copies of EAAAK can be omitted
EAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAK

SEQ ID NO: 950 - protein, 102 aa, synthetic construct
  VARIANT 2..101: note = Wherein up to 19 copies of EAAAK can be omitted
AEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKA

SEQ ID NO: 951 - protein, 206 aa, synthetic construct
  VARIANT 2..101: note = Wherein up to 19 copies of EAAAK can be omitted
  VARIANT 106..205: note = Wherein up to 19 copies of EAAAK can be omitted
AEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKALEAEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKA

SEQ ID NO: 952 - protein, 40 aa, synthetic construct
  VARIANT 1..40: note = Wherein up to 19 copies of KP can be omitted
KPKPKPKPKPKPKPKPKPKPKPKPKPKPKPKPKPKPKPKP

SEQ ID NO: 953 - protein, 40 aa, synthetic construct
  VARIANT 1..40: note = Wherein up to 19 copies of EP can be omitted
EPEPEPEPEPEPEPEPEPEPEPEPEPEPEPEPEPEPEPEP

SEQ ID NO: 954 - protein, 60 aa, synthetic construct
  VARIANT 1..60: note = Wherein up to 19 copies of TPR can be omitted
TPRTPRTPRTPRTPRTPRTPRTPRTPRTPRTPRTPRTPRTPRTPRTPRTPRTPRTPRTPR

SEQ ID NO: 955 - protein, 40 aa, synthetic construct
  VARIANT 1..40: note = Wherein up to 19 copies of AP can be omitted
APAPAPAPAPAPAPAPAPAPAPAPAPAPAPAPAPAPAPAP

SEQ ID NO: 956 - protein, 5 aa, synthetic construct
EAAAK

SEQ ID NO: 957 - skipped

SEQ ID NO: 958 - protein, 10 aa, synthetic construct
EAAAKEAAAK

SEQ ID NO: 959 - protein, 15 aa, synthetic construct
EAAAKEAAAKEAAAK

SEQ ID NO: 960 - protein, 46 aa, synthetic construct
AEAAAKEAAAKEAAAKEAAAKALEAEAAAKEAAAKEAAAKEAAAKA

SEQ ID NO: 961 - protein, 12 aa, synthetic construct
AEAAAKEAAAKA

SEQ ID NO: 962 - protein, 4 aa, synthetic construct
APAP

SEQ ID NO: 963 - protein, 8 aa, synthetic construct
APAPAPAP

SEQ ID NO: 964 - protein, 12 aa, synthetic construct
APAPAPAPAPAP

SEQ ID NO: 965 - protein, 16 aa, synthetic construct
APAPAPAPAPAPAPAP

SEQ ID NO: 966 - skipped

SEQ ID NO: 967 - protein, 4 aa, synthetic construct
KPKP

SEQ ID NO: 968 - protein, 8 aa, synthetic construct
KPKPKPKP

SEQ ID NO: 969 - protein, 12 aa, synthetic construct
KPKPKPKPKPKP

SEQ ID NO: 970 - protein, 16 aa, synthetic construct
KPKPKPKPKPKPKPKP

SEQ ID NO: 971 - skipped

SEQ ID NO: 972 - protein, 4 aa, synthetic construct
EPEP

SEQ ID NO: 973 - protein, 8 aa, synthetic construct
EPEPEPEP

SEQ ID NO: 974 - protein, 12 aa, synthetic construct
EPEPEPEPEPEP

SEQ ID NO: 975 - protein, 16 aa, synthetic construct
EPEPEPEPEPEPEPEP

SEQ ID NO: 976 - skipped

SEQ ID NO: 977 - protein, 5 aa, synthetic construct
GCGGS

SEQ ID NO: 978 - protein, 55 aa, synthetic construct
  VARIANT 6..55: note = Wherein up to 9 copies of GGGGS can be omitted
GCGGSGGGGSGGGGSGGGGSGGGGSGGGGSGGGGSGGGGSGGGGSGGGGSGGGGS

SEQ ID NO: 979 - protein, 5 aa, synthetic construct
CGGGS

SEQ ID NO: 980 - protein, 55 aa, synthetic construct
  VARIANT 6..55: note = Wherein up to 9 copies of GGGGS can be omitted
CGGGSGGGGSGGGGSGGGGSGGGGSGGGGSGGGGSGGGGSGGGGSGGGGSGGGGS

SEQ ID NO: 981 - protein, 5 aa, synthetic construct
GGCGS

SEQ ID NO: 982 - protein, 55 aa, synthetic construct
  VARIANT 6..55: note = Wherein up to 9 copies of GGGGS can be omitted
GGCGSGGGGSGGGGSGGGGSGGGGSGGGGSGGGGSGGGGSGGGGSGGGGSGGGGS

SEQ ID NO: 983 - protein, 5 aa, synthetic construct
GGGCS

SEQ ID NO: 984 - protein, 55 aa, synthetic construct
  VARIANT 6..55: note = Wherein up to 9 copies of GGGGS can be omitted
GGGCSGGGGSGGGGSGGGGSGGGGSGGGGSGGGGSGGGGSGGGGSGGGGSGGGGS

SEQ ID NO: 985 - protein, 5 aa, synthetic construct
GGGGC

SEQ ID NO: 986 - protein, 55 aa, synthetic construct
  VARIANT 6..55: note = Wherein up to 9 copies of GGGGS can be omitted
GGGGCGGGGSGGGGSGGGGSGGGGSGGGGSGGGGSGGGGSGGGGSGGGGSGGGGS

SEQ ID NO: 987 - protein, 586 aa, Homo sapiens
LAGETGQEAAPLDGVLANPPNISSLSPRQLLGFPCAEVSGLSTERVRELAVALAQKNVKLSTEQLRCLAHRLSEPPEDLDALPLDLLLFLNPDAFSGPQACTRFFSRITKANVDLLPRGAPERQRLLPAALACWGVRGSLLSEADVRALGGLACDLPGRFVAESAEVLLPRLVSCPGPLDQDQQEAARAALQGGGPPYGPPSTWSVSTMDALRGLLPVLGQPIIRSIPQGIVAAWRQRSSRDPSWRQPERTILRPRFRREVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVNKGHEMSPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSMQEALSGTPCLLGPGPVLTVLALLLASTLA

SEQ ID NO: 988 - protein, 10 aa, Homo sapiens
KLLGPHVEGL

SEQ ID NO: 989 - protein, 10 aa, Homo sapiens
AFYPGYLCSL

SEQ ID NO: 990 - protein, 9 aa, Homo sapiens
VLPLTVAEV

SEQ ID NO: 991 - protein, 9 aa, Homo sapiens
ELAVALAQK

SEQ ID NO: 992 - protein, 9 aa, Homo sapiens
ALQGGGPPY

SEQ ID NO: 993 - protein, 9 aa, Homo sapiens
FYPGYLCSL

SEQ ID NO: 994 - protein, 9 aa, Homo sapiens
LYPKARLAF

SEQ ID NO: 995 - protein, 15 aa, Homo sapiens
LLFLLFSLGWVGPSR

SEQ ID NO: 996 - protein, 15 aa, Homo sapiens
VNKGHEMSPQAPRRP

SEQ ID NO: 997 - protein, 15 aa, Homo sapiens
FMKLRTDAVLPLTVA

SEQ ID NO: 998 - protein, 10 aa, Homo sapiens
DAALLATQMD

SEQ ID NO: 999 - protein, 1055 aa, Homo sapiens
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTSIISAVVGILLVVVLGVVFGILIKRRQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLDHVRENRGRLGSQDLLNWCMQIAKGMSYLEDVRLVHRDLAARNVLVKSPNHVKITDFGLARLLDIDETEYHADGGKVPIKWMALESILRRRFTHQSDVWSYGVTVWELMTFGAKPYDGIPAREIPDLLEKGERLPQPPICTIDVYMIMVKCWMIDSECRPRFRELVSEFSRMARDPQRFVVIQNEDLGPASPLDSTFYRSLLEDDDMGDLVDAEEYLVPQQGFFCPDPAPGAGGMVHHRHRSSSTRNM

SEQ ID NO: 1000 - protein, 184 aa, Pan troglodytes [sequence omitted]

SEQ ID NO: 1001 - protein, 502 aa, Homo sapiens
MDFLLLQDPASTCVPEPASQHTLRSGPGCLQQPEQQGVRDPGGIWAKLGAAEASAERLQGRRSRGASGSEPQQMGSDVRDLNALLPAVPSLGGGGGCALPVSGAAQWAPVLDFAPPGASAYGSLGGPAPPPAPPPPPPPPPHSFIKQEPSWGGAEPHEEQCLSAFTVHFSGQFTGTAGACRYGPFGPPPPSQASSGQARMFPNAPYLPSCLESQPAIRNQGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKGHSTGYESDNHTTPILCGAQYRIHTHGVFRGIQDVRRVPGVAPTLVRSASETSEKRPFMCAYPGCNKRYFKLSHLQMHSRKHTGEKPYQCDFKDCERRFSRSDQLKRHQRRHTGVKPFQCKTCQRKFSRSDHLKTHTRTHTGEKPFSCRWPSCQKKFARSDELVRHHNMHQRNMTKLQLAL

SEQ ID NO: 1002 - protein, 9 aa, Homo sapiens
RMFPNAPYL

SEQ ID NO: 1003 - protein, 8 aa, Homo sapiens
CMTWNQMN

SEQ ID NO: 1004 - protein, 9 aa, Homo sapiens
CYTWNQMNL

SEQ ID NO: 1005 - protein, 15 aa, Homo sapiens
CMTWNQMNLGATLKG

SEQ ID NO: 1006 - protein, 15 aa, Homo sapiens
WNQMNLGATLKGVAA

SEQ ID NO: 1007 - protein, 15 aa, Homo sapiens
CMTWNYMNLGATLKG

SEQ ID NO: 1008 - protein, 15 aa, Homo sapiens
WNYMNLGATLKGVAA

SEQ ID NO: 1009 - protein, 15 aa, Homo sapiens
MTWNQMNLGATLKGV

SEQ ID NO: 1010 - protein, 15 aa, Homo sapiens
TWNQMNLGATLKGVA

SEQ ID NO: 1011 - protein, 15 aa, Homo sapiens
CMTWNLMNLGATLKG

SEQ ID NO: 1012 - protein, 15 aa, Homo sapiens
MTWNLMNLGATLKGV

SEQ ID NO: 1013 - protein, 15 aa, Homo sapiens
TWNLMNLGATLKGVA

SEQ ID NO: 1014 - protein, 15 aa, Homo sapiens
WNLMNLGATLKGVAA

SEQ ID NO: 1015 - protein, 8 aa, Homo sapiens
MNLGATLK

SEQ ID NO: 1016 - protein, 15 aa, Homo sapiens
MTWNYMNLGATLKGV

SEQ ID NO: 1017 - protein, 15 aa, Homo sapiens
TWNYMNLGATLKGVA

SEQ ID NO: 1018 - protein, 17 aa, Homo sapiens
CMTWNQMNLGATLKGVA

SEQ ID NO: 1019 - protein, 17 aa, Homo sapiens
CMTWNLMNLGATLKGVA

SEQ ID NO: 1020 - protein, 17 aa, Homo sapiens
CMTWNYMNLGATLKGVA

SEQ ID NO: 1021 - protein, 9 aa, Homo sapiens
GYLRNPTAC

SEQ ID NO: 1022 - protein, 9 aa, Homo sapiens
GALRNPTAL

SEQ ID NO: 1023 - protein, 9 aa, Homo sapiens
YALRNPTAC

SEQ ID NO: 1024 - protein, 9 aa, Homo sapiens
GLLRNPTAC

SEQ ID NO: 1025 - protein, 9 aa, Homo sapiens
RYRPHPGAL

SEQ ID NO: 1026 - protein, 9 aa, Homo sapiens
YQRPHPGAL

SEQ ID NO: 1027 - protein, 9 aa, Homo sapiens
RLRPHPGAL

SEQ ID NO: 1028 - protein, 9 aa, Homo sapiens
RIRPHPGAL

SEQ ID NO: 1029 - protein, 15 aa, Homo sapiens
QFPNHSFKHEDPMGQ

SEQ ID NO: 1030 - protein, 9 aa, Homo sapiens
HSFKHEDPY

SEQ ID NO: 1031 - protein, 13 aa, Homo sapiens
QFPNHSFKHEDPM

SEQ ID NO: 1032 - protein, 13 aa, Homo sapiens
QFPNHSFKHEDPY

SEQ ID NO: 1033 - protein, 13 aa, Homo sapiens
KRPFMCAYPGCNK

SEQ ID NO: 1034 - protein, 13 aa, Homo sapiens
KRPFMCAYPGCYK

SEQ ID NO: 1035 - protein, 9 aa, Homo sapiens
FMCAYPGCY

SEQ ID NO: 1036 - protein, 9 aa, Homo sapiens
FMCAYPGCK

SEQ ID NO: 1037 - protein, 15 aa, Homo sapiens
KRPFMCAYPGCNKRY

SEQ ID NO: 1038 - protein, 15 aa, Homo sapiens
SEKRPFMCAYPGCNK

SEQ ID NO: 1039 - protein, 15 aa, Homo sapiens
KRPFMCAYPGCYKRY

SEQ ID NO: 1040 - protein, 9 aa, Homo sapiens
NLMNLGATL

SEQ ID NO: 1041 - protein, 9 aa, Homo sapiens
NYMNLGATL

SEQ ID NO: 1042 - protein, 9 aa, Human papillomavirus
KLPQLCTEL

SEQ ID NO: 1043 - protein, 9 aa, Human papillomavirus
LQTTIHDII

SEQ ID NO: 1044 - protein, 9 aa, Human papillomavirus
VYDFAFRDL

SEQ ID NO: 1045 - protein, 9 aa, Human papillomavirus
FAFRDLCIV

SEQ ID NO: 1046 - protein, 9 aa, Human papillomavirus
KFYSKISEY

SEQ ID NO: 1047 - protein, 9 aa, Human papillomavirus
ISEYRHYCY

SEQ ID NO: 1048 - protein, 9 aa, Human papillomavirus
TLHEYMLDL

SEQ ID NO: 1049 - protein, 9 aa, Human papillomavirus
YMLDLQPET

SEQ ID NO: 1050 - protein, 9 aa, Human papillomavirus
QAEPDRAHY

SEQ ID NO: 1051 - protein, 9 aa, Human papillomavirus
RAHYNIVTF

SEQ ID NO: 1052 - protein, 9 aa, Human papillomavirus
CDSTLRLCV

SEQ ID NO: 1053 - protein, 10 aa, Human papillomavirus
LCVQSTHVDI

SEQ ID NO: 1054 - protein, 9 aa, Human papillomavirus
LLMGTLGIV

SEQ ID NO: 1055 - protein, 8 aa, Human papillomavirus
TLGIVCPI

SEQ ID NO: 1056 - protein, 12 aa, Human papillomavirus
LLMGTLGIVCPI

SEQ ID NO: 1057 - protein, 261 aa, Homo sapiens
MAVTACQGLGFVVSLIGIAGIIAATCMDQWSTQDLYNNPVTAVFNYQGLWRSCVRESSGFTECRGYFTLLGLPAMLQAVRALMIVGIVLGAIGLLVSIFALKCIRIGSMEDSAKANMTLTSGIMFIVSGLCAIAGVSVFANMLVTNFWMSTANMYTGMGGMVQTVQTRYTFGAALFVGWVAGGLTLIGGVMMCIACRGLAPEETNYKAVSYHASGHSVAYKPGGFKASTGFGSNTKNKKIYDGGARTEDEVQSYPSKHDYV

SEQ ID NO: 1058 - protein, 15 aa, Homo sapiens
TEDEVQSYPSKHDYV

SEQ ID NO: 1059 - protein, 12 aa, Homo sapiens
EVQSYPSKHDYV

SEQ ID NO: 1060 - protein, 293 aa, Homo sapiens
QDNCTCPTNKMTVCSPDGPGGRCQCRALGSGMAVDCSTLTSKCLLLKARMSAPKNARTLVRPSEHALVDNDGLYDPDCDPEGRFKARQCNQTSVCWCVNSVGVRRTDKGDLSLRCDELVRTHHILIDLRHRPTAGAFNHSDLDAELRRLFRERYRLHPKFVAAVHYEQPTIQIELRQNTSQKAAGDVDIGDAAYYFERDIKGESLFQGRGGLDLRVRGEPLQVERTLIYYLDEIPPKFSMKRLTAGLIAVIVVVVVALVAGMAVLVITNRRKSGKYKKVEIKELGELRKEPSL

SEQ ID NO: 1061 - protein, 214 aa, Homo sapiens
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1062 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1063 - protein, 107 aa, synthetic construct [sequence omitted]

SEQ ID NO: 1064 - protein, 120 aa, synthetic construct [sequence omitted]

SEQ ID NO: 1065 - protein, 90 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAED

SEQ ID NO: 1066 - protein, 137 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKTAVYYCSRWGGDGFYAMDYWGQGTLVTVSS

SEQ ID NO: 1067 - protein, 11 aa, synthetic construct
RASQDVNTAVA

SEQ ID NO: 1068 - protein, 6 aa, synthetic construct
SASFLY

SEQ ID NO: 1069 - protein, 8 aa, synthetic construct
QQHYTTPP

SEQ ID NO: 1070 - protein, 8 aa, synthetic construct
GFNIKDTY

SEQ ID NO: 1071 - protein, 8 aa, synthetic construct
IYPTNGYT

SEQ ID NO: 1072 - protein, 13 aa, synthetic construct
SRWGGDGFYAMDY

SEQ ID NO: 1073 - protein, 242 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIK

SEQ ID NO: 1074 - protein, 214 aa, synthetic construct
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1075 - protein, 448 aa, synthetic construct
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1076 - protein, 107 aa, Homo sapiens [sequence omitted]

SEQ ID NO: 1077 - protein, 119 aa, Homo sapiens [sequence omitted]

SEQ ID NO: 1078 - protein, 11 aa, Homo sapiens
KASQDVSIGVA

SEQ ID NO: 1079 - protein, 6 aa, Homo sapiens
SASYRY

SEQ ID NO: 1080 - protein, 8 aa, Homo sapiens
QQYYIYPY

SEQ ID NO: 1081 - protein, 10 aa, Homo sapiens
GFTFTDYTMD

SEQ ID NO: 1082 - protein, 18 aa, Homo sapiens
ADVNPNSGGSIYNQRFKG

SEQ ID NO: 1083 - protein, 12 aa, Homo sapiens
ARNLGPSFYFDY

SEQ ID NO: 1084 - protein, 15 aa, synthetic construct
KASQSVDYDGDSYLN

SEQ ID NO: 1085 - protein, 7 aa, synthetic construct
DASNLVS

SEQ ID NO: 1086 - protein, 9 aa, synthetic construct
QQSTEDPWT

SEQ ID NO: 1087 - protein, 5 aa, synthetic construct
SYWMN

SEQ ID NO: 1088 - protein, 17 aa, synthetic construct
QIWPGDGDTNYNGKFKG

SEQ ID NO: 1089 - protein, 15 aa, synthetic construct
RETTTVGRYYYAMDY

SEQ ID NO: 1090 - protein, 249 aa, synthetic construct
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVS

SEQ ID NO: 1091 - protein, 217 aa, synthetic construct
DIALTQPASVSGSPGQSITISCTGTSSDIGGYNSVSWYQQHPGKAPKLMIYGVNNRPSGVSNRFSGSKSGNTASLTISGLQAEDEADYYCSSYDIESATPVFGGGTKLTVLGQPKAAPSVTLFPPSSEELQANKATLVCLISDFYPGAVTVAWKGDSSPVKAGVETTTPSKQSNNKYAASSYLSLTPEQWKSHRSYSCQVTHEGSTVEKTVAPTESS

SEQ ID NO: 1092 - protein, 450 aa, synthetic construct
QVELVQSGAEVKKPGESLKISCKGSGYSFTSYWIGWVRQAPGKGLEWMGIIDPGDSRTRYSPSFQGQVTISADKSISTAYLQWSSLKASDTAMYYCARGQLYGGTYMDGWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1093 - protein, 107 aa, synthetic construct
DIALTQPASVSGSPGQSITISCTGTSSDIGGYNSVSWYQQHPGKAPKLMIYGVNNRPSGVSNRFSGSKSGNTASLTISGLQAEDEADYYCSSYDIESATPVFGGGTK

SEQ ID NO: 1094 - protein, 120 aa, synthetic construct
QVELVQSGAEVKKPGESLKISCKGSGYSFTSYWIGWVRQAPGKGLEWMGIIDPGDSRTRYSPSFQGQVTISADKSISTAYLQWSSLKASDTAMYYCARGQLYGGTYMDGWGQGTLVTVSS

SEQ ID NO: 1095 - protein, 14 aa, synthetic construct
TGTSSDIGGYNSVS

SEQ ID NO: 1096 - protein, 11 aa, synthetic construct
LMIYGVNNRPS

SEQ ID NO: 1097 - protein, 10 aa, synthetic construct
SSYDIESATP

SEQ ID NO: 1098 - protein, 10 aa, synthetic construct
GYSFTSYWIG

SEQ ID NO: 1099 - protein, 16 aa, synthetic construct
WMGIIDPGDSRTRYSP

SEQ ID NO: 1100 - protein, 11 aa, synthetic construct
GQLYGGTYMDG

SEQ ID NO: 1101 - protein, 244 aa, synthetic construct
QVQLQQSGAEVKKPGASVKVSCKASGYTFTGYYMHWVRQAPGQGLEWMGRINPNSGGTNYAQKFQGRVTMTRDTSISTAYMELSRLRSEDTAVYYCARGRYYGMDVWGQGTMVTVSSGGGGSGGGGSGGGGSGGGGSEIVLTQSPATLSLSPGERATISCRASQSVSSNFAWYQQRPGQAPRLLIYDASNRATGIPPRFSGSGSGTDFTLTISSLEPEDFAAYYCHQRSNWLYTFGQGTKVDIK

SEQ ID NO: 1102 - protein, 253 aa, synthetic construct
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARDLRRTVVTPRAYYGMDVWGQGTTVTVSSGGGGSGGGGSGGGGSGGGGSDIQLTQSPSTLSASVGDRVTITCQASQDISNSLNWYQQKAGKAPKLLIYDASTLETGVPSRFSGSGSGTDFSFTISSLQPEDIATYYCQQHDNLPLTFGQGTKVEIK

SEQ ID NO: 1103 - protein, 109 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPIFTFGPGTKVDIK

SEQ ID NO: 1104 - protein, 124 aa, synthetic construct
QMQLVESGGGVVQPGRSLRLSCTASGFTFSNNGMHWVRQAPGKGLEWVAVIWFDGMNKFYVDSVKGRFTISRDNSKNTLYLEMNSLRAEDTAIYYCAREGDGSGIYYYYGMDVWGQGTTVTVSS

SEQ ID NO: 1105 - protein, 106 aa, synthetic construct
DIELTQSPAIMSASPGEKVTMTCSASSSVSYMHWYQQKSGTSPKRWIYDTSKLASGVPGRFSGSGSGNSYSLTISSVEAEDDATYYCQQWSKHPLTFGSGTKVEIK

SEQ ID NO: 1106 - protein, 119 aa, synthetic construct
QVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGSGTPVTVSS

SEQ ID NO: 1107 - protein, 112 aa, synthetic construct
DIALTQPASVSGSPGQSITISCTGTSSDIGGYNSVSWYQQHPGKAPKLMIYGVNNRPSGVSNRFSGSKSGNTASLTISGLQAEDEADYYCSSYDIESATPVFGGGTKLTVLG

SEQ ID NO: 1108 - protein, 106 aa, synthetic construct
DIQMTQSPSSLSASVGDRVTITCSASSSVSYMHWYQQKSGKAPKLLIYDTSKLASGVPSRFSGSGSGTDFTLTISSLQPEDFATYYCQQWSKHPLTFGQGTKLEIK

SEQ ID NO: 1109 - protein, 119 aa, synthetic construct
QVQLVQSGAEVKKPGASVKVSCKASGYSFTGYTMNWVRQAPGQGLEWMGLITPYNGASSYNQKFRGKATMTVDTSTSTVYMELSSLRSEDTAVYYCARGGYDGRGFDYWGQGTLVTVSS

SEQ ID NO: 1110 - protein, 10 aa, synthetic construct
GDSVSSNSAA

SEQ ID NO: 1111 - protein, 9 aa, synthetic construct
TYYRSKWYN

SEQ ID NO: 1112 - protein, 14 aa, synthetic construct
AREVTGDLEDAFDI

SEQ ID NO: 1113 - protein, 6 aa, synthetic construct
QTIWSY

SEQ ID NO: 1114 - protein, 9 aa, synthetic construct
QQSYSIPQT

SEQ ID NO: 1115 - protein, 11 aa, synthetic construct
KASQDVSIAVA

SEQ ID NO: 1116 - protein, 7 aa, synthetic construct
SASYRYT

SEQ ID NO: 1117 - protein, 9 aa, synthetic construct
QQHYITPLT

SEQ ID NO: 1118 - protein, 5 aa, synthetic construct
NYGMN

SEQ ID NO: 1119 - protein, 17 aa, synthetic construct
WINTYTGEPTYTDDFKG

SEQ ID NO: 1120 - protein, 12 aa, synthetic construct
GGFGSSYWYFDV

SEQ ID NO: 1121 - protein, 5 aa, synthetic construct
TAGMQ

SEQ ID NO: 1122 - protein, 17 aa, synthetic construct
WINTHSGVPKYAEDFKG

SEQ ID NO: 1123 - protein, 12 aa, synthetic construct
SGFGSSYWYFDV

SEQ ID NO: 1124 - protein, 11 aa, synthetic construct
KASQDVSTAVA

SEQ ID NO: 1125 - protein, 107 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIK

SEQ ID NO: 1126 - protein, 121 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 1127 - protein, 107 aa, synthetic construct
DIQMTQSPSSLSASVGDRVTITCKASQDVSTAVAWYQQKPGKAPKLLIYSASYRYTGVPSRFSGSGSGTDFTLTISSLQPEDFAVYYCQQHYITPLTFGQGTKLEIK

SEQ ID NO: 1128 - protein, 121 aa, synthetic construct
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFDVWGQGTLVTVSS

SEQ ID NO: 1129 - protein, 217 aa, synthetic construct
QSVLTQPPSASGTPGQRVTISCSGSSSNIGSNTVNWYQQLPGTAPKLLIFNYHQRPSGVPDRFSGSKSGSSASLAISGLQSEDEADYYCAAWDDSLNGWVFGGGTKLTVLGQPKAAPSVTLFPPSSEELQANKATLVCLISDFYPGAVTVAWKADSSPVKAGVETTTPDSKQSNNKYAASSYLSLTPEQWKSHRSYSCQVTHEGSTVEKTVAPTECS

SEQ ID NO: 1130 - skipped

SEQ ID NO: 1131 - protein, 111 aa, synthetic construct
QSVLTQPPSASGTPGQRVTISCSGSSSNIGSNTVNWYQQLPGTAPKLLIFNYHQRPSGVPDRFSGSKSGSSASLAISGLQSEDEADYYCAAWDDSLNGWVFGGGTKLTVLG

SEQ ID NO: 1132 - protein, 131 aa, synthetic construct
EVQLVESGGGLVKPGGSLRLSCAASGFTFGDYALSWFRQAPGKGLEWVGVSRSKAYGGTTDYAASVKGRFTISRDDSKSTAYLQMNSLKTEDTAVYYCASSGYSSGWTPFDYWGQGTLVTVSSASTKGPSV

SEQ ID NO: 1133 - protein, 8 aa, synthetic construct
SSNIGSNT

SEQ ID NO: 1134 - protein, 11 aa, synthetic construct
AAWDDSLNGWV

SEQ ID NO: 1135 - protein, 8 aa, synthetic construct
GFTFGDYA

SEQ ID NO: 1136 - protein, 10 aa, synthetic construct
SRSKAYGGTT

SEQ ID NO: 1137 - protein, 14 aa, synthetic construct
ASSGYSSGWTPFDY

SEQ ID NO: 1138 - protein, 249 aa, synthetic construct
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYNGYDVLDNWGQGTLVTVSSGGGGSGGGGSGGGGSGGGGSDIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKR

SEQ ID NO: 1139 - protein, 249 aa, synthetic construct
DIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKRGGGGSGGGGSGGGGSGGGGSQVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYNGYDVLDNWGQGTLVTVSS

SEQ ID NO: 1140 - protein, 11 aa, synthetic construct
SASQDISNYLN

SEQ ID NO: 1141 - protein, 7 aa, synthetic construct
YTSNLHS

SEQ ID NO: 1142 - protein, 9 aa, synthetic construct
QQYRKLPWT

SEQ ID NO: 1143 - protein, 5 aa, synthetic construct
NYWMH

SEQ ID NO: 1144 - protein, 17 aa, synthetic construct
ATYRGHSDTYYNQKFKG

SEQ ID NO: 1145 - protein, 12 aa, synthetic construct
GAIYNGYDVLDN

SEQ ID NO: 1146 - protein, 108 aa, synthetic construct
DIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKR

SEQ ID NO: 1147 - protein, 121 aa, synthetic construct
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYDGYDVLDNWGQGTLVTVSS

SEQ ID NO: 1148 - protein, 20 aa, synthetic construct
VTSAPDTRPAPGSTAPPAHG

SEQ ID NO: 1149 - protein, 43 aa, synthetic construct
SNIKFRPGSVVVQLTLAFREGTINVHDVETQFNQYKTEAASRY

SEQ ID NO: 1150 - protein, 35 aa, synthetic construct
SVVVQLTLAFREGTINVHDVETQFNQYKTEAASRY

SEQ ID NO: 1151 - protein, 20 aa, synthetic construct
LAFREGTINVHDVETQFNQY

SEQ ID NO: 1152 - protein, 25 aa, synthetic construct
SNIKFRPGSVVVQLTLAAFREGTIN

SEQ ID NO: 1153 - protein, 5 aa, synthetic construct
RYGMS

SEQ ID NO: 1154 - protein, 17 aa, synthetic construct
TISGGGTYIYYPDSVKG

SEQ ID NO: 1155 - protein, 13 aa, synthetic construct
DNYGRNYDYGMDY

SEQ ID NO: 1156 - protein, 10 aa, synthetic construct
SATSSVSYIH

SEQ ID NO: 1157 - protein, 7 aa, synthetic construct
STSNLAS

SEQ ID NO: 1158 - protein, 9 aa, synthetic construct
QQRSSSPFT

SEQ ID NO: 1159 - protein, 5 aa, synthetic construct
GYAMS

SEQ ID NO: 1160 - protein, 17 aa, synthetic construct
TISSGGTYIYYPDSVKG

SEQ ID NO: 1161 - protein, 12 aa, synthetic construct
LGGDNYYEYFDV

SEQ ID NO: 1162 - protein, 15 aa, synthetic construct
RASKSVSTSGYSYMH

SEQ ID NO: 1163 - protein, 7 aa, synthetic construct
LASNLES

SEQ ID NO: 1164 - protein, 9 aa, synthetic construct
QHSRELPFT

SEQ ID NO: 1165 - protein, 5 aa, synthetic construct
DYAMN

SEQ ID NO: 1166 - protein, 17 aa, synthetic construct
VISTFSGNINFNQKFKG

SEQ ID NO: 1167 - protein, 10 aa, synthetic construct
SDYYGPYFDY

SEQ ID NO: 1168 - protein, 16 aa, synthetic construct
RSSQTIVHSNGNTYLE

SEQ ID NO: 1169 - protein, 7 aa, synthetic construct
KVSNRFS

SEQ ID NO: 1170 - protein, 9 aa, synthetic construct
FQGSHVPFT

SEQ ID NO: 1171 - protein, 9 aa, synthetic construct
LGGDNYYEY

SEQ ID NO: 1172 - protein, 15 aa, synthetic construct
TASKSVSTSGYSYMH

SEQ ID NO: 1173 - protein, 7 aa, synthetic construct
LVSNLES

SEQ ID NO: 1174 - protein, 10 aa, synthetic construct
QHIRELTRSE

SEQ ID NO: 1175 - protein, 8 aa, synthetic construct
GFTFSNYY

SEQ ID NO: 1176 - protein, 8 aa, synthetic construct
ISGRGSTI

SEQ ID NO: 1177 - protein, 10 aa, synthetic construct
VKDRGGYSPY

SEQ ID NO: 1178 - protein, 6 aa, synthetic construct
QSISTY

SEQ ID NO: 1179 - protein, 10 aa, synthetic construct
QQSYSTPPIT

SEQ ID NO: 1180 - protein, 8 aa, synthetic construct
GYTFTDYS

SEQ ID NO: 1181 - protein, 8 aa, synthetic construct
INTETGVP

SEQ ID NO: 1182 - protein, 8 aa, synthetic construct
ARRTGFDY

SEQ ID NO: 1183 - protein, 11 aa, synthetic construct
KNLLHSDGITY

SEQ ID NO: 1184 - protein, 9 aa, synthetic construct
VQVLELPFT

SEQ ID NO: 1185 - protein, 8 aa, synthetic construct
GFTFSSYA

SEQ ID NO: 1186 - protein, 8 aa, synthetic construct
ISDGGSYS

SEQ ID NO: 1187 - protein, 14 aa, synthetic construct
ARDSYYDNSYVRDY

SEQ ID NO: 1188 - protein, 6 aa, synthetic construct
QDINTF

SEQ ID NO: 1189 - protein, 9 aa, synthetic construct
LQYDEFPLT

SEQ ID NO: 1190 - protein, 9 aa, synthetic construct
SLLMWITQC

SEQ ID NO: 1191 - protein, 115 aa, synthetic construct
  VARIANT 20: note = wherein amino acid 20 can be V or A
  VARIANT 51: note = wherein amino acid 51 can be Q, P, S, T, or M
  VARIANT 52: note = wherein amino acid 52 can be S, P, F, or G
  VARIANT 53: note = wherein amino acid 53 can be S, W, H, or T
  VARIANT 94: note = wherein amino acid 94 can be P, H, or A
  VARIANT 95: note = wherein amino acid 95 can be T, L, M, A, Q, Y, E, I, F, V, N, G, S, D, or R
  VARIANT 96: note = wherein amino acid 96 can be S, L, T, Y, I, Q, V, E, A, W, R, G, H, D, or K
  VARIANT 97: note = wherein amino acid 97 can be G, D, N, V, S, T, or A
  VARIANT 98: note = wherein amino acid 98 can be G, P, H, S, T, W, or A
  VARIANT 99: note = wherein amino acid 99 can be S, T, Y, D, H, V, N, E, G, Q, K, A, I, or R
  VARIANT 100: note = wherein amino acid 100 can be Y, F, M, or D
  VARIANT 101: note = wherein amino acid 101 can be I, P, T, or M
  VARIANT 103: note = wherein amino acid 103 can be T or A
MQEVTQIPAALSVPEGENLVLNCSFTDSAIYNLQWFRQDPGKGLTSLLLIQSSQREQTSGRLNASLDKSSGRSTLYIAASQPGDSATYLCAVRPTSGGSYIPTFGRGTSLIVHPY

SEQ ID NO: 1192 - protein, 112 aa, synthetic construct
  VARIANT 18: note = wherein amino acid 18 can be M or V
  VARIANT 50: note = wherein amino acid 50 can be G, V, or I
  VARIANT 52: note = wherein amino acid 52 can be G or Q
  VARIANT 53: note = wherein amino acid 53 can be I, T, or M
  VARIANT 55: note = wherein amino acid 55 can be D or R
  VARIANT 56: note = wherein amino acid 56 can be Q or R
  VARIANT 70: note = wherein amino acid 70 can be T or I
  VARIANT 94: note = wherein amino acid 94 can be Y, N, or F
  VARIANT 95: note = wherein amino acid 95 can be V or L
  VARIANT 97: note = wherein amino acid 97 can be N, G, or D
MGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVL

SEQ ID NO: 1193 - protein, 115 aa, synthetic construct
MQEVTQIPAALSVPEGENLVLNCSFTDSAIYNLQWFRQDPGKGLTSLLLIMSHQREQTSGRLNASLDKSSGRSTLYIAASQPGDSATYLCAVRPTSGGSYIPTFGRGTSLIVHPY

SEQ ID NO: 1194 - protein, 112 aa, synthetic construct
MGVTQTPKFQVLKTGQSMTLQCAQDMNHEYMSWYRQDPGMGLRLIHYSVSAGITDQGEVPNGYNVSRSTTEDFPLRLLSAAPSQTSVYFCASSYVGNTGELFFGEGSRLTVL

SEQ ID NO: 1195 - protein, 9 aa, synthetic construct
YIIFVYIPL

SEQ ID NO: 1196 - protein, 10 aa, synthetic construct
TIHEIILECV

SEQ ID NO: 1197 - protein, 9 aa, synthetic construct
KCIDFYSRI

SEQ ID NO: 1198 - protein, 9 aa, synthetic construct
FQQLFLNTL

SEQ ID NO: 1199 - skipped

SEQ ID NO: 1200 - protein, 133 aa, synthetic construct
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASSFWGRSTDTQYFGPGTRLTVL

SEQ ID NO: 1201 - protein, 9 aa, synthetic construct
YLQPRTLIL

SEQ ID NO: 1202 - protein, 9 aa, synthetic construct
YLQPRTLVL

SEQ ID NO: 1203 - protein, 13 aa, synthetic construct
GCGGSGGGSGGGS

SEQ ID NO: 1204 - protein, 8 aa, synthetic construct
LEVLFQGP

SEQ ID NO: 1205 - protein, 7 aa, synthetic construct
ENLYTQS

SEQ ID NO: 1206 - protein, 5 aa, synthetic construct
DDDDK

SEQ ID NO: 1207 - protein, 4 aa, synthetic construct
LVPR

SEQ ID NO: 1208 - protein, 22 aa, synthetic construct
GSGATNFSLLKQAGDVEENPGP

SEQ ID NO: 1209 - protein, 909 aa, synthetic construct
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 1210 - protein, 902 aa, synthetic construct
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAPAPAPAPAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 1211 - protein, 1055 aa, synthetic construct
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAPAPAPAPAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 1212 - protein, 1215 aa, synthetic construct
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 1213 - protein, 1208 aa, synthetic construct
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAPAPAPAPAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 1214 - protein, 1054 aa, synthetic construct
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSAPAPGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAPAPAPAPAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 1215 - protein, 1058 aa, synthetic construct
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSAPAPAPAPGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAPAPAPAPAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 1216 - protein, 1062 aa, synthetic construct
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSAPAPAPAPAPAPGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAPAPAPAPAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 1217 - protein, 301 aa, synthetic construct
  VARIANT 31: note = Wherein X can be any amino acid other than His
  VARIANT 57: note = Wherein X can be any amio acid other than Phe
  VARIANT 184: note = Wherein X can be any amino acid other than His
  VARIANT 210: note = Wherein X can be any amio acid other than Phe
GGGGSGGGGSGGGGSAPTSSSTKKTQLQLEXLLLDLQMILNGINNYKNPKLTRMLTXKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEXLLLDLQMILNGINNYKNPKLTRMLTXKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 1218 - protein, 126 aa, synthetic construct
  VARIANT 1..12: note = Wherein X is any amino acid and up to 8 copies of X can be deleted
XXXXXXXXXXXXGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 1219 - protein, 532 aa, synthetic construct
AAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 1220 - protein, 123 aa, synthetic construct
VMPLSAPTLGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 1221 - protein, 1067 aa, synthetic construct
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 1222 - protein, 1072 aa, synthetic construct
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 1223 - protein, 1077 aa, synthetic construct
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 1224 - protein, 813 aa, synthetic construct
APTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1225 - protein, 133 aa, synthetic construct
  VARIANT 38: note = wherein X is any amino acid other than Arg
APTSSSTKKTQLQLEHLLLDLQMILNGINNYKNPKLTXMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 1226 - protein, 133 aa, synthetic construct
  VARIANT 42: note = wherein X is any amino acid other than Phe
APTSSSTKKTQLQLEHLLLDLQMILNGINNYKNPKLTRMLTXKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 1227 - protein, 133 aa, synthetic construct
  VARIANT 43: note = wherein X is any amino acid other than Lys
APTSSSTKKTQLQLEHLLLDLQMILNGINNYKNPKLTRMLTFXFYMPKKATELKHLQCLEEELKPLEEVLNLAQSKNFHLRPRDLISNINVIVLELKGSETTFMCEYADETATIVEFLNRWITFCQSIISTLT

SEQ ID NO: 1228 - protein, 133 aa, synthetic construct
  VARIANT 45: note = wherein X is any amino acid other than Tyr
APTSSSTKKTQLQLEHLLLDLQMILNGINNYKNPKLTRMLTFKFXMPKKATELKHLQCLEEELKPLEEVLNLAQSKNFHLRPRDLISNINVIVLELKGSETTFMCEYADETATIVEFLNRWITFCQSIISTLT

SEQ ID NO: 1229 - protein, 133 aa, synthetic construct
  VARIANT 62: note = wherein X is any amino acid other than Glu
APTSSSTKKTQLQLEHLLLDLQMILNGINNYKNPKLTRMLTFKFYMPKKATELKHLQCLEEXLKPLEEVLNLAQSKNFHLRPRDLISNINVIVLELKGSETTFMCEYADETATIVEFLNRWITFCQSIISTLT

SEQ ID NO: 1230 - protein, 133 aa, synthetic construct
  VARIANT 65: note = wherein X is any amino acid other than Pro
APTSSSTKKTQLQLEHLLLDLQMILNGINNYKNPKLTRMLTFKFYMPKKATELKHLQCLEEELKXLEEVLNLAQSKNFHLRPRDLISNINVIVLELKGSETTFMCEYADETATIVEFLNRWITFCQSIISTLT

SEQ ID NO: 1231 - protein, 133 aa, synthetic construct
  VARIANT 68: note = wherein X is any amino acid other than Glu
APTSSSTKKTQLQLEHLLLDLQMILNGINNYKNPKLTRMLTFKFYMPKKATELKHLQCLEEELPKLEXVLNLAQSKNFHLRPRDLISNINVIVLELKGSETTFMCEYADETATIVEFLNRWITFCQSIISTLT

SEQ ID NO: 1232 - protein, 133 aa, synthetic construct
  VARIANT 69: note = wherein X is any amino acid other than Val
APTSSSTKKTQLQLEHLLLDLQMILNGINNYKNPKLTRMLTFKFYMPKKATELKHLQCLEEELPKLEEXLNLAQSKNFHLRPRDLISNINVIVLELKGSETTFMCEYADETATIVEFLNRWITFCQSIISTLT

SEQ ID NO: 1233 - protein, 133 aa, synthetic construct
  VARIANT 72: note = wherein X is any amino acid other than Leu
APTSSSTKKTQLQLEHLLLDLQMILNGINNYKNPKLTRMLTFKFYMPKKATELKHLQCLEEELPKLEEVLNXAQSKNFHLRPRDLISNINVIVLELKGSETTFMCEYADETATIVEFLNRWITFCQSIISTLT

SEQ ID NO: 1234 - protein, 133 aa, synthetic construct
  VARIANT 15: note = wherein X is any amino acid other than Glu
APTSSSTKKTQLQLXHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 1235 - protein, 133 aa, synthetic construct
  VARIANT 16: note = wherein X is any amino acid other than His
APTSSSTKKTQLQLEXLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 1236 - protein, 133 aa, synthetic construct
  VARIANT 19: note = wherein X is any amino acid other than Leu
APTSSSTKKTQLQLEHLLXDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 1237 - protein, 133 aa, synthetic construct
  VARIANT 20: note = wherein X is any amino acid other than Asp
APTSSSTKKTQLQLEHLLLXLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 1238 - protein, 133 aa, synthetic construct
  VARIANT 84: note = wherein X is any amino acid other than Asp
APTSSSTKKTQLQLEHLLLDLQMILNGINNYKNPKLTRMLTFKFYMPKKATELKHLQCLEEELKPLEEVLNLAQSKNFHLRPRXLISNINVIVLELKGSETTFMCEYADETATIVEFLNRWITFCQSIISTLT

SEQ ID NO: 1239 - protein, 133 aa, synthetic construct
  VARIANT 87: note = wherein X is any amino acid other than Ser
APTSSSTKKTQLQLEHLLLDLQMILNGINNYKNPKLTRMLTFKFYMPKKATELKHLQCLEEELKPLEEVLNLAQSKNFHLRPRDLIXNINVIVLELKGSETTFMCEYADETATIVEFLNRWITFCQSIISTLT

SEQ ID NO: 1240 - protein, 133 aa, synthetic construct
  VARIANT 88: note = wherein X is any amino acid other than Asn
APTSSSTKKTQLQLEHLLLDLQMILNGINNYKNPKLTRMLTFKFYMPKKATELKHLQCLEEELKPLEEVLNLAQSKNFHLRPRDLISXINVIVLELKGSETTFMCEYADETATIVEFLNRWITFCQSIISTLT

SEQ ID NO: 1241 - protein, 133 aa, synthetic construct
  VARIANT 91: note = wherein X is any amino acid other than Val
APTSSSTKKTQLQLEHLLLDLQMILNGINNYKNPKLTRMLTFKFYMPKKATELKHLQCLEEELKPLEEVLNLAQSKNFHLRPRDLISNINXIVLELKGSETTFMCEYADETATIVEFLNRWITFCQSIISTLT

SEQ ID NO: 1242 - protein, 133 aa, synthetic construct
  VARIANT 92: note = wherein X is any amino acid other than Ile
APTSSSTKKTQLQLEHLLLDLQMILNGINNYKNPKLTRMLTFKFYMPKKATELKHLQCLEEELKPLEEVLNLAQSKNFHLRPRDLISNINVXVLELKGSETTFMCEYADETATIVEFLNRWITFCQSIISTLT

SEQ ID NO: 1243 - protein, 133 aa, synthetic construct
  VARIANT 16: note = Wherein X can be, e.g., Ala, Glu, Thr, or Asp, but any of the following substitutions also may be employed: Gly, Val, Leu, Ile, Arg, Asn, Cys, Glu, Gln, Ile, Lys, Met, Phe, Pro, Ser, Tyr, or Trp
  VARIANT 42: note = wherein X can be, e.g., Ala or Lys, but any of the following substitutions also may be employed: Met, Pro, Ser, Thr, Trp, Tyr, or Val
APTSSSTKKTQLQLEXLLLDLQMILNGINNYKNPKLTRMLTXKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 1244 - protein, 133 aa, synthetic construct
APTSSSTKKTQLQLEELLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 1245 - protein, 133 aa, synthetic construct
APTSSSTKKTQLQLEDLLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT